SEQUENCE LISTING (34 sequences)

SEQ ID NO: 1 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 2 - protein, 478 aa, Rattus norvegicus
MASLRPFFILALLALVSLADQESCKGRCTQGFMASKKCQCDELCTYYQSCCVDYMEQCKPQVTRGDVFTMPEDEYWSYDYPEETKNSTSTGVQSENTSLHFNLKPRAEETIKPTTPDPQEQSNTQEPEVGQQGVAPRPDTTDEGTSEFPEEELCSGKPFDAFTDLKNGSLFAFRGEYCYELDETAVRPGYPKLIQDVWGIEGPIDAAFTRINCQGKTYLFKGSQYWRFEDGVLDPDYPRNISEGFSGIPDNVDAALALPAHSYSGRERVYFFKGKQYWEYEFQQQPSQEECEGSSLSAVFEHFALLQRDSWENIFELLFWGRSSDGAKGPQFISRDWHGVPGKVDAAMAGRIYITGSTFRSVQAKKQKSGRRSRKRYRSRRGRGHSRSRSRSMSSRRPSRSVWFSLLSSEESGLGTYNYDYDMNWRIPATCEPIQSVYFFSGDKYYRVNLRTRRVDSVNPPYPRSIAQYWLGCPTSEK

SEQ ID NO: 3 - protein, 478 aa, Mus musculus
MAPLRPFFILALVAWVSLADQESCKGRCTQGFMASKKCQCDELCTYYQSCCADYMEQCKPQVTRGDVFTMPEDDYWSYDYVEEPKNNTNTGVQPENTSPPGDLNPRTDGTLKPTAFLDPEEQPSTPAPKVEQQEEILRPDTTDQGTPEFPEEELCSGKPFDAFTDLKNGSLFAFRGQYCYELDETAVRPGYPKLIQDVWGIEGPIDAAFTRINCQGKTYLFKGSQYWRFEDGVLDPGYPRNISEGFSGIPDNVDAAFALPAHRYSGRERVYFFKGKQYWEYEFQQQPSQEECEGSSLSAVFEHFALLQRDSWENIFELLFWGRSSDGAREPQFISRNWHGVPGKVDAAMAGRIYVTGSLSHSAQAKKQKSKRRSRKRYRSRRGRGHRRSQSSNSRRSSRSIWFSLFSSEESGLGTYNNYDYDMDWLVPATCEPIQSVYFFSGDKYYRVNLRTRRVDSVNPPYPRSIAQYWLGCPTSEK

SEQ ID NO: 4 - protein, 747 aa, Homo sapiens
MGLRTAKKRGLGGGGKWKREEGGGTRGRREVRPACFLQSGGRGDPGDVGGPAGNPGCSPHPRAATRPPPLPAHTPAHTPEWCGAASAEAAEPRRAGPHLCIPAPGLTKTPILEKVPRKMAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHVTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQ

SEQ ID NO: 5 - protein, 640 aa, Rattus norvegicus
MPILEKAPQKMPVKASSWEELDLPKLPVPPLQQTLATYLQCMQHLVPEEQFRKSQAIVKRFGAPGGLGETLQEKLLERQEKTANWVSEYWLNDMYLNNRLALPVNSSPAVIFARQHFQDTNDQLRFAACLISGVLSYKTLLDSHSLPTDWAKGQLSGQPLCMKQYYRLFSSYRLPGHTQDTLVAQKSSIMPEPEHVIVACCNQFFVLDVVINFRRLSEGDLFTQLRKIVKMASNEDERLPPIGLLTSDGRSEWAKARTVLLKDSTNRDSLDMIERCICLVCLDGPGTGELSDTHRALQLLHGGGCSLNGANRWYDKSLQFVVGRDGTCGVVCEHSPFDGIVLVQCTEHLLKHMMTSNKKLVRADSVSELPAPRRLRLKCSPETQGHLASSAEKLQRIVKNLDFIVYKFDNYGKTFIKKQKYSPDGFIQVALQLAYYRLYQRLVPTYESASIRRFQEGRVDNIRSATPEALAFVQAMTDHKAAMPASEKLQLLQTAMQAHKQYTVMAITGMAIDNHLLALRELARDLCKEPPEMFMDETYLMSNRFVLSTSQVPTTMEMFCCYGPVVPNGNGACYNPQPEAITFCISSFHSCKETSSVEFAEAVGASLVDMRDLCSSRQPADSKPPAPKEKARGPSQAKQS

SEQ ID NO: 6 - protein, 641 aa, Mus musculus
MPILEKVPPKMPVQASSCEEVLDLPKLPVPPLQQTLATYLQCMQHLVPEEQFRKSQAIVKRFGAPGGLGETLQEKLLERQEKTANWVSEYWLNDMYLNNRLALPVNSSPAVIFARQHFQDTNDQLRFAASLISGVLSYKALLDSQSIPTDWAKGQLSGQPLCMKQYYRLFSSYRLPGHTQDTLVAQKSSIMPEPEHVIVACCNQFFVLDVVINFRRLSEGDLFTQLRKIVKMASNEDERLPPIGLLTSDGRSEWAKARTVLLKDSTNRDSLDMIERCICLVCLDGPGTGDLSDTHRALQLLHGGGCSLNGANRWYDKSLQFVVGRDGTCGVVCEHSPFDGIVLVQCTEHLLKHMMTGNKKLVRVDSVSELPAPRRLRWKCSPETQGHLASSAEKLQRIVKNLDFIVYKFDNYGKTFIKKQKCSPDGFIQVALQLAYYRLYQRLVPTYESASIRRFQEGRVDNIRSATPEALAFVQAMTDHKAAVLASEKLQLLQRAIQAQTEYTVMAITGMAIDNHLLALRELARDLCKEPPEMFMDETYLMSNRFILSTSQVPTTMEMFCCYGPVVPNGYGACYNPHAEAITFCISSFHGCKETSSVEFAEAVGASLVDMRDLCSSRQPADSKPPTAKERARGPSQAKQS

SEQ ID NO: 7 - protein, 420 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFC

SEQ ID NO: 8 - protein, 422 aa, Rattus norvegicus
MDVFSFGQGNNTTASQEPFGTGGNVTSISDVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVRKVEKKGAGTSLGTSSAPPPKKSLNGQPGSGDWRRCAENRAVGTPCTNGAVRQGDDEATLEVIEVHRVGNSKEHLPLPSESGSNSYAPACLERKNERNAEAKRKMALARERKTVKTLGIIMGTFILCWLPFFIVALVLPFCESSCHMPALLGAIINWLGYSNSLLNPVIYAYFNKDFQNAFKKIIKCKFCRR

SEQ ID NO: 9 - protein, 421 aa, Mus musculus
MDMFSLGQGNNTTTSLEPFGTGGNDTGLSNVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSNPNECTISKDHGYTIYSTFGAFYIPLLLMLVLYGRIFRAARFRIRKTVKKVEKKGAGTSFGTSSAPPPKKSLNGQPGSGDCRRSAENRAVGTPCANGAVRQGEDDATLEVIEVHRVGNSKGHLPLPSESGATSYVPACLERKNERTAEAKRKMALARERKTVKTLGIIMGTFILCWLPFFIVALVLPFCESSCHMPELLGAIINWLGYSNSLLNPVIYAYFNKDFQNAFKKIIKCKFCR

SEQ ID NO: 10 - protein, 1434 aa, Homo sapiens
MEDHMFGVQQIQPNVISVRLFKRKVGGLGFLVKERVSKPPVIISDLIRGGAAEQSGLIQAGDIILAVNGRPLVDLSYDSALEVLRGIASETHVVLILRGPEGFTTHLETTFTGDGTPKTIRVTQPLGPPTKAVDLSHQPPAGKEQPLAVDGASGPGNGPQHAYDDGQEAGSLPHANGLAPRPPGQDPAKKATRVSLQGRGENNELLKEIEPVLSLLTSGSRGVKGGAPAKAEMKDMGIQVDRDLDGKSHKPLPLGVENDRVFNDLWGKGNVPVVLNNPYSEKEQPPTSGKQSPTKNGSPSKCPRFLKVKNWETEVVLTDTLHLKSTLETGCTEYICMGSIMHPSQHARRPEDVRTKGQLFPLAKEFIDQYYSSIKRFGSKAHMERLEEVNKEIDTTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKPPRGRFDVLPLLLQANGNDPELFQIPPELVLEVPIRHPKFEWFKDLGLKWYGLPAVSNMLLEIGGLEFSACPFSGWYMGTEIGVRDYCDNSRYNILEEVAKKMNLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKVMSMEEYDIVHLEHETLVLVVTSTFGNGDPPENGEKFGCALMEMRHPNSVQEERKSYKVRFNSVSSYSDSQKSSGDGPDLRDNFESAGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTFVAEAPELTQGLSNVHKKRVSAARLLSRQNLQSPKSSRSTIFVRLHTNGSQELQYQPGDHLGVFPGNHEDLVNALIERLEDAPPVNQMVKVELLEERNTALGVISNWTDELRLPPCTIFQAFKYYLDITTPPTPLQLQQFASLATSEKEKQRLLVLSKGLQEYEEWKWGKNPTIVEVLEEFPSIQMPATLLLTQLSLLQPRYYSISSSPDMYPDEVHLTVAIVSYRTRDGEGPIHHGVCSSWLNRIQADELVPCFVRGAPSFHLPRNPQVPCILVGPGTGIAPFRSFWQQRQFDIQHKGMNPCPMVLVFGCRQSKIDHIYREETLQAKNKGVFRELYTAYSREPDKPKKYVQDILQEQLAESVYRALKEQGGHIYVCGDVTMAADVLKAIQRIMTQQGKLSAEDAGVFISRMRDDNRYHEDIFGVTLRTYEVTNRLRSESIAFIEESKKDTDEVFSS

SEQ ID NO: 11 - protein, 1429 aa, Rattus norvegicus
MEENTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGPPTKAVDLSHQPSASKDQSLAVDRVTGLGNGPQHAQGHGQGAGSVSQANGVAIDPTMKSTKANLQDIGEHDELLKEIEPVLSILNSGSKATNRGGPAKAEMKDTGIQVDRDLDGKSHKAPPLGGDNDRVFNDLWGKDNVPVILNNPYSEKEQSPTSGKQSPTKNGSPSRCPRFLKVKNWETDVVLTDTLHLKSTLETGCTEHICMGSIMLPSQHTRKPEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKAPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKPNNSLISNDRSWKRNKFRLTYVAEAPDLTQGLSNVHKKRVSAARLLSRQNLQSPKFSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDADEVFSS

SEQ ID NO: 12 - protein, 1429 aa, Mus musculus
MEEHTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGTPTKAVDLSRQPSASKDQPLAVDRVPGPSNGPQHAQGRGQGAGSVSQANGVAIDPTMKNTKANLQDSGEQDELLKEIEPVLSILTGGGKAVNRGGPAKAEMKDTGIQVDRDLDGKLHKAPPLGGENDRVFNDLWGKGNVPVVLNNPYSENEQSPASGKQSPTKNGSPSRCPRFLKVKNWETDVVLTDTLHLKSTLETGCTEQICMGSIMLPSHHIRKSEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVEFTEICIQQGWKPPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTYVAEAPELTQGLSNVHKKRVSAARLLSRQNLQSPKSSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDTDEVFSS

SEQ ID NO: 13 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 14 - protein, 430 aa, Rattus norvegicus
MERRRITSARRSYASSETMVRGHGPTRHLGTIPRLSLSRMTPPLPARVDFSLAGALNAGFKETRASERAEMMELNDRFASYIEKVRFLEQQNKALAAELNQLRAKEPTKLADVYQAELRELRLRLDQLTTNSARLEVERDNLTQDLGTLRQKLQDETNLRLEAENNLAVYRQEADEATLARVDLERKVESLEEEIQFLRKIHEEEVRELQEQLAQQQVHVEMDVAKPDLTAALREIRTQYEAVATSNMQETEEWYRSKFADLTDVASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 15 - protein, 430 aa, Mus musculus
MERRRITSARRSYASETVVRGLGPSRQLGTMPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNFAQDLGTLRQKLQDETNLRLEAENNLAAYRQEADEATLARVDLERKVESLEEEIQFLRKIYEEEVRELREQLAQQQVHVEMDVAKPDLTAALREIRTQYEAVATSNMQETEEWYRSKFADLTDAASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKDSKQEHKDVVM

SEQ ID NO: 16 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 17 - protein, 466 aa, Rattus norvegicus
MAEEQDLSEVELSPVGSEEPRCLSPSSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGETDQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLSSALAVASGHSAWISKPPGVALPTVSPPAVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 18 - protein, 466 aa, Mus musculus
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLGSALAVASGHSAWISKPPGVALPTVSPPGVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 19 - protein, 80 aa, synthetic construct
MGRAMVARLGLGLLLLALLLPTQIYSSETTTGTSSNSSQSTSNSGLAPNPTNATTKAAGGALQSTASLFVVSLSLLHLYS

SEQ ID NO: 20 - protein, 347 aa, unidentified
MGNEEPWVQPALEMPKRRDILAIVLIVLPWTLLITVWHQSTLAPLLAVHKDEGSDPRRETPPGADPREYCTSDRDIVEVVRTEYVYTRPPPWSDTLPTIHVVTPTYSRPVQKAELTRMANTLLHVPNLHWLVVEDAPRRTPLTARLLRDTGLNYTHLHVETPRNYKLRGDARDPRIPRGTMQRNLALRWLRETFPRNSSQPGVVYFADDDNTYSLELFEEMRSTRRVSVWPVAFVGGLRYEAPRVNGAGKVVGWKTVFDPHRPFAIDMAGFAVNLRLILQRSQAYFKLRGVKGGYQESSLLRELVTLNDLEPKAANCTKILVWHTRTEKPVLVNEGKKGFTDPSVEI

SEQ ID NO: 21 - protein, 347 aa, unidentified; identical to SEQ ID NO: 20

SEQ ID NO: 22 - protein, 334 aa, unidentified
MPKRRDILAIVLIVLPWTLLITVWHQSTLAPLLAVHKDEGSDPRRETPPGADPREYCTSDRDIVEVVRTEYVYTRPPPWSDTLPTIHVVTPTYSRPVQKAELTRMANTLLHVPNLHWLVVEDAPRRTPLTARLLRDTGLNYTHLHVETPRNYKLRGDARDPRIPRGTMQRNLALRWLRETFPRNSSQPGVVYFADDDNTYSLELFEEMRSTRRVSVWPVAFVGGLRYEAPRVNGAGKVVGWKTVFDPHRPFAIDMAGFAVNLRLILQRSQAYFKLRGVKGGYQESSLLRELVTLNDLEPKAANCTKILVWHTRTEKPVLVNEGKKGFTDPSVEI

SEQ ID NO: 23 - protein, 334 aa, unidentified; identical to SEQ ID NO: 22

SEQ ID NO: 24 - protein, 238 aa, unidentified
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 25 - protein, 708 aa, unidentified
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMREGPQMMLLLTDARPSNHFLSPLLSLHRG

SEQ ID NO: 26 - protein, 313 aa, unidentified
MWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 27 - protein, 385 aa, unidentified
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 28 - protein, 1363 aa, unidentified
MTRGGGSGSSRGSRDRVAARWGWAPLAPPREAPARSGTRPPRGSRARLRRVAAAAAAAAMSPGKPGAGGAGTRRTGWRRRRRRRRQEAATTVPGLGRTAGPDSRVRGTFQGARGMKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRACVAGFTNITFHPQNKGLIDGAESYVSFSRYSDAVSLPQDPGVICGAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 29 - protein, 410 aa, unidentified
MLLIIVIIVIVNYCDCTCVTDKMCIFFTAAVDWIMPFGIRMLLRAHKPGSSRRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 30 - protein, 1350 aa, unidentified
MSVNLQGDQRGATEARTFLLEIQPVSQFLEESAFSQSGPGAVICGLSTKVGVSSSKISRLGGRSKEREVGMDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 31 - protein, 334 aa, unidentified
MVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTVSRHGFEQTTDVLPFILKSSLGASCEPTAFPLPPAAPGPCAHLFIWMLAECTPCSPVWSSIS

SEQ ID NO: 32 - protein, 160 aa, unidentified
MLLLLLSIIVLHVAVLVLLFVSTIVSQWIVGNGHATDLWQNCSTSSSGNVHHCFSSSPNEWLQSVQATMILSIIFSILSLFLFFCQLFTLTKGGRFYITGIFQILAGLCVMSAAAIYTVRHPEWHLNSDYSYGFAYILAWVAFPLALLSGVIYVILRKRE

SEQ ID NO: 33 - protein, 408 aa, unidentified
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 34 - protein, 288 aa, unidentified
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

(19) United States
(12) Patent Application Publication
(10) Pub. No.: US 2025/0099508 A1
(43) Pub. Date: Mar. 27, 2025

(54) SYSTEMS, CELL LINES AND METHODS OF PRODUCING AND USING THE SAME

(71) Applicant: THE REGENTS OF THE UNIVERSITY OF CALIFORNIA, Oakland, CA (US)

(72) Inventor: Faranak FATTAHI, Redwood City, CA (US)

(73) Assignee: THE REGENTS OF THE UNIVERSITY OF CALIFORNIA, Oakland, CA (US)

(21) Appl. No.: 18/726,674

(22) PCT Filed: Jan. 3, 2023
(86) PCT No.: PCT/US2023/060054

§ 371 Date: Jul. 3, 2024

Related U.S. Application Data
(60) Provisional application No. 63/296,157, filed on Jan. 3, 2022.

(51) Int. Cl. A61K 35/30; C12N 5/079

(52) U.S. Cl. CPC A61K 35/30; C12N 5/0618; C12N 2501/999

(57) ABSTRACT

The present disclosure relates generally to two-dimensional and three-dimensional cultures of cells, the cultures comprising one or a plurality of enteric neurons and/or one or a plurality of enteric glial cells. These cells and cultures can be used for screening compounds for neurologically active compounds, for transplantation into subjects for animal models, and for administration to a subject with a gut motility disorder.

### CROSS-REFERENCE TO RELATED APPLICATIONS

This application claims priority to U.S. Provisional Application Ser. No. 63/296,157, which was filed Jan. 3, 2022, in accordance with 35 USC 119 (e), and which is incorporated by reference in its entirety.

### STATEMENT REGARDING FEDERALLY SPONSORED RESEARCH

This invention was made with government support under grant numbers DP2NS116769 and R01CA240984 awarded by the National Institute of Health (NIH), and grant number R01DK121169 awarded by the National Institute of Diabetes and Digestive and Kidney Diseases. The government has certain rights in the invention.

### REFERENCE TO AN ELECTRONIC SEQUENCE LISTING

The contents of the electronic sequence listing (37944_0027P1.xml; Size: 49,238 bytes; and Date of Creation: Dec. 30, 2022) is hereby incorporated by reference in its entirety.

### TECHNOLOGY FIELD

The present disclosure relates generally to compositions comprising glial cells, methods of culturing pluripotent stem cells in defined conditions, inducing the pluripotent stem cells to differentiate into enteric neuronal cells and glial cells, which, in turn, are components of either 2D or 3D cell cultures. The disclosure also relates to cultured two-dimensional neuronal cell-containing cultures and three-dimensional spheroids, and method of using the same.

### BACKGROUND

The enteric nervous system (ENS) is the largest and most complex division of the autonomic nervous system (De Giorgio, 2006). More than 500 million enteric neurons and roughly seven times as many enteric glia form interconnected enteric ganglia embedded in two distinct layers within the gut wall: the myenteric plexus residing between the longitudinal and circular muscles, and the submucosal plexus residing between the circular muscle and the mucosa (Grubišić and Gulbransen, 2017; Grundmann et al., 2019; Hamnett et al., 2021; Sasselli et al., 2012).

The ENS, uniquely, is not dependent on input from the central nervous system (CNS) to command GI tract functions (Furness et al., 2014). This autonomy is exemplified by studies in which segments of the bowel removed from the body continue to generate complex motor patterns ex vivo. ENS autonomy is the result of extraordinarily diverse neuronal and glial cell types with distinct neurochemical signatures working together in harmony (Brehmer, 2021; Qu et al., 2008; Fung and Vanden Berghe, 2020). Thus, the ENS is equipped to control complex gut functions including motility, secretion, absorption, blood flow regulation and barrier function support. Furthermore, the ENS communicates extrinsically with the CNS, enteroendocrine system, immune system, and the gut microbiome in order to maintain vitality and proper gut homeostasis (Furness et al., 2014; Long-Smith et al., 2020; Muller et al., 2014; Obata and Pachnis, 2016; Schneider et al., 2019; Yoo and Mazmanian, 2017).

The neurochemical and functional complexity of the ENS resemble the CNS (Gershon, 1999), yet much slower progress has been made in the field of ENS research. Despite being the largest and most complex division of the peripheral nervous system and playing a central role in the development and progression of enteric neuropathies and diseases of the gut-brain axis, ENS research has been disproportionately affected by multiple longstanding technical challenges. For example, enteric neurons are diluted throughout the GI tract, comprising less than 1% of gut tissue (Drokhlyansky et al., 2020). Therefore, scientists must rely on samples collected during GI resection surgeries, rather than more routine GI biopsies, to access ENS tissue. Furthermore, it is difficult to isolate the ENS without significant sampling bias related to harsh tissue dissociation techniques that damage fragile neurites, and reliable surface markers suitable for FACS purification of enteric neurons and glia are lacking.

The complex developmental processes and the elaborate cellular architecture of the ENS, as well as its remarkable communication with the rest of the body provide a wide array of possibilities for abnormalities to arise. Comprising some of the most challenging clinical disorders, enteric neuropathies, also known as disorders of gut brain interaction (DGBI), result from loss, degeneration or functional impairment of the ENS cell types (De Giorgio et al., 2016; Niesler et al., 2021). Our incomplete understanding of ENS development and function is accountable for the long-term morbidity and mortality of GI disorders and limited availability of therapeutic interventions.

### SUMMARY

The disclosure relates to neuronal cell lines and cell cultures comprising the same. The cell cultures comprise enteric neurons disclosed herein or glial cells disclosed herein or a combination of the both the enteric neuronal cells and glial cells. In some embodiments, the disclosure relates to a method of making and culturing enteric neuronal cells and glial cells disclosed herein. The resulting cultures are suitable for screening potential therapeutic agents for the treatment of enteric neuropathies such as gastroparesis, esophageal achalasia, chronic intestinal pseudo-obstruction, and hypertrophic pyloric stenosis, and applications in regenerative medicine. In some embodiments, the disclosure relates to compositions comprising cell lines and cultures comprising neuronal cells, and, in some embodiments, those compositions are for transplantation in or administration to a mammalian subject.

In one aspect, the disclosure relates to a method of inducing nitric oxide sensitive enteric neurons comprising exposing one or a plurality of enteric neurons to one or a series of cell culture medium disclosed herein in combination with a platelet-derived growth hormone receptor inhibitor disclosed herein.

In another aspect, the disclosure relates to a method of enriching a cell population for subtypes of intergenic neurons comprising exposing the one or plurality of iPSCs to a PDFGR inhibitor.

In another aspect, the disclosure relates to a composition comprising a spheroid comprising enteric neurons, wherein the enteric neurons comprise SOX10 and CD24.

In another aspect, the disclosure relates to a composition comprising a spheroid comprising enteric neurons, wherein the enteric neurons comprise SOX10 and CD45RA.

In another aspect, the disclosure relates to a composition comprising a spheroid comprising enteric neurons, wherein the enteric neurons comprise SOX10 and CD57.

In another aspect, the disclosure relates to a composition comprising a spheroid comprising enteric neurons, wherein the enteric neurons comprise SOX10 and CD63.

In yet another aspect, the disclosure relates to a composition comprising a spheroid comprising enteric neurons, wherein the enteric neurons comprise SOX10 and CD71.

In yet another aspect, the disclosure relates to a composition comprising a spheroid comprising enteric neurons, wherein the enteric neurons comprise SOX10 and CD121b.

In yet another aspect, the disclosure relates to a composition comprising a spheroid comprising enteric neurons, wherein the enteric neurons comprise SOX10 and CD147.

In yet another aspect, the disclosure relates to a composition comprising a spheroid comprising enteric neurons, wherein the enteric neurons comprise SOX10 and CD164.

In yet another aspect, the disclosure relates to a composition comprising a spheroid comprising enteric neurons, wherein the enteric neurons comprise SOX10 and CD184.

In yet another aspect, the disclosure relates to a composition comprising a spheroid comprising enteric neurons, wherein the enteric neurons comprise SOX10 and CD193.

In yet another aspect, the disclosure relates to a composition comprising a spheroid comprising enteric neurons, wherein the enteric neurons comprise SOX10 and CD243.

In yet another aspect, the disclosure relates to a composition comprising a spheroid comprising enteric neurons, wherein the enteric neurons comprise SOX10 and CD275.

In another aspect, the disclosure relates to a composition comprising spheroid comprising enteric neurons, wherein the enteric neurons comprise SOX10 and at least one or a combination of CD24, CD45RA, CD57, CD63, CD71, CD121b, CD147, CD164, CD184, CD193, CD243, CD275.

In another aspect, the disclosure relates to a method of differentiating a neuronal cell to an enteric neuronal cell, the method comprising exposing an effective amount of a platelet-derived growth factor receptor (PDGFR) inhibitor or a pharmaceutically acceptable salt thereof to a neuronal cell for a time period sufficient to differentiate the neuronal cell to an enteric neuronal cell.

In some embodiments, the PDGFR inhibitor is selected from (Z)-orantinib, AC710, AC710 mesylate, AG 1295, amuvatinib, amuvatinib hydrochloride, avapritinib, axitinib, AZD2932, cediranib, cediranib maleate, chiauranib, CHIR-124, CP-673451, crenolanib, dovitinib, dovitinib lactate, dovitinib lactate hydrate, dovitinib-D8, ENMD-2076, ENMD-2076 tartrate, flumatinib, flumatinib mesylate, GZD856, GZD856 formic, HG-7-85-01, hypothemycrin, ilorasertib, ilorasertib hydrochloride, imatinib, imatinib D4, imatinib D8, imatinib mesylate, JI-101, JNJ-10198409, KG5, Ki20227, lenvatinib, lenvatinib mesylate, linifanib, masitinib, masitinib mesylate, methylnissolin, multi-kinase inhibitor 1, N-(p-coumaroyl) serotonin, nintedanib, nintedanib esylate, NVP-ACC789, orantinib, pazopanib, pazopanib hydrochloride, PD-089828, PD-161570, PDGFRα kinase inhibitor 1, ponatinib, ponatinib D8, PP121, PP58, regorafenib, regorafenib D3, regorafenib hydrochloride, regorafenib monohydrate, ripretinib, sennoside B, seralutinib, SU5402, SU14813, SU14813 maleate, SU16f, SU4312, SU4984, sunitinib, sunitinib D10, sunitinib malate, sunitinib-d4, TAK-593, tandutinib, tandutinib hydrochloride, telatinib, telatinib mesylate, TG 100572, TG 100572 hydrochloride, TG 100801, TG 100801 hydrochloride, toceranib, toceranib phosphate, toceranib-d8, trapidil, tyrosine kinase-IN-1, tyrphostin AG1296, tryphostin AG1433, and vorolanib.

In some embodiments, the PDGFR inhibitor is selected from:

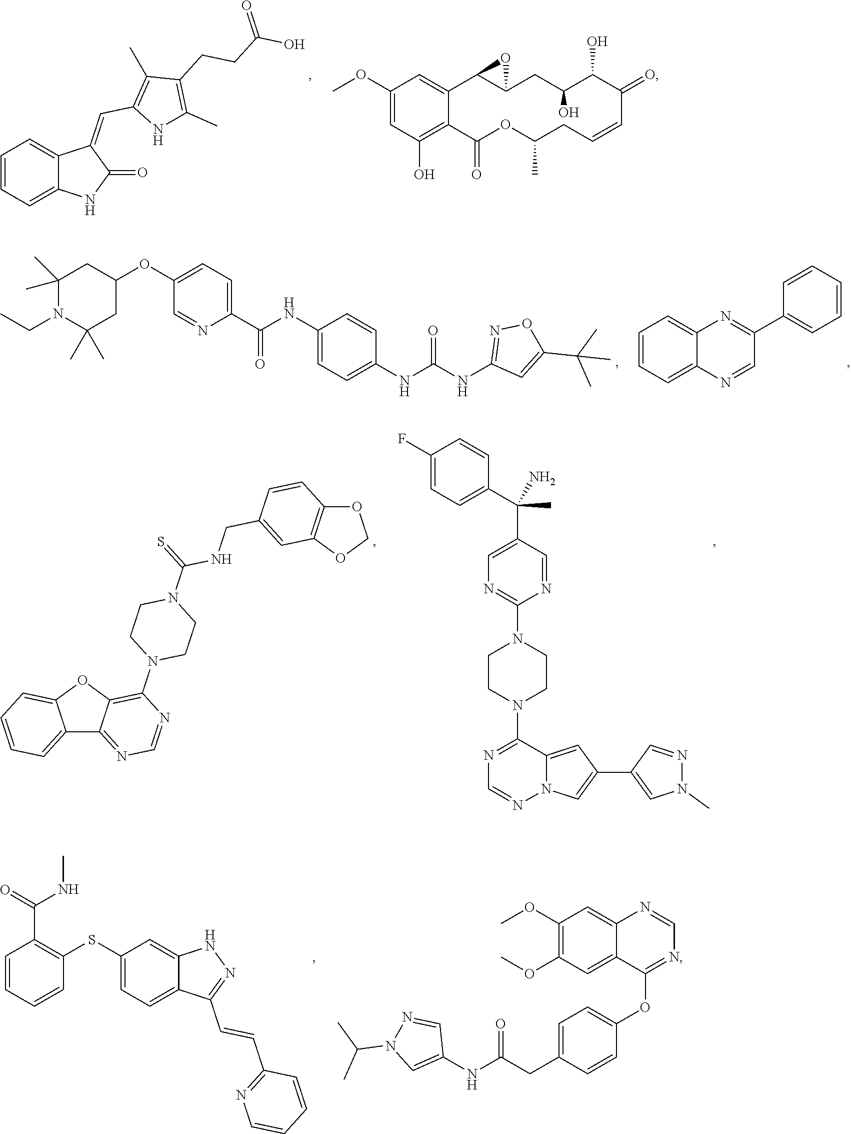

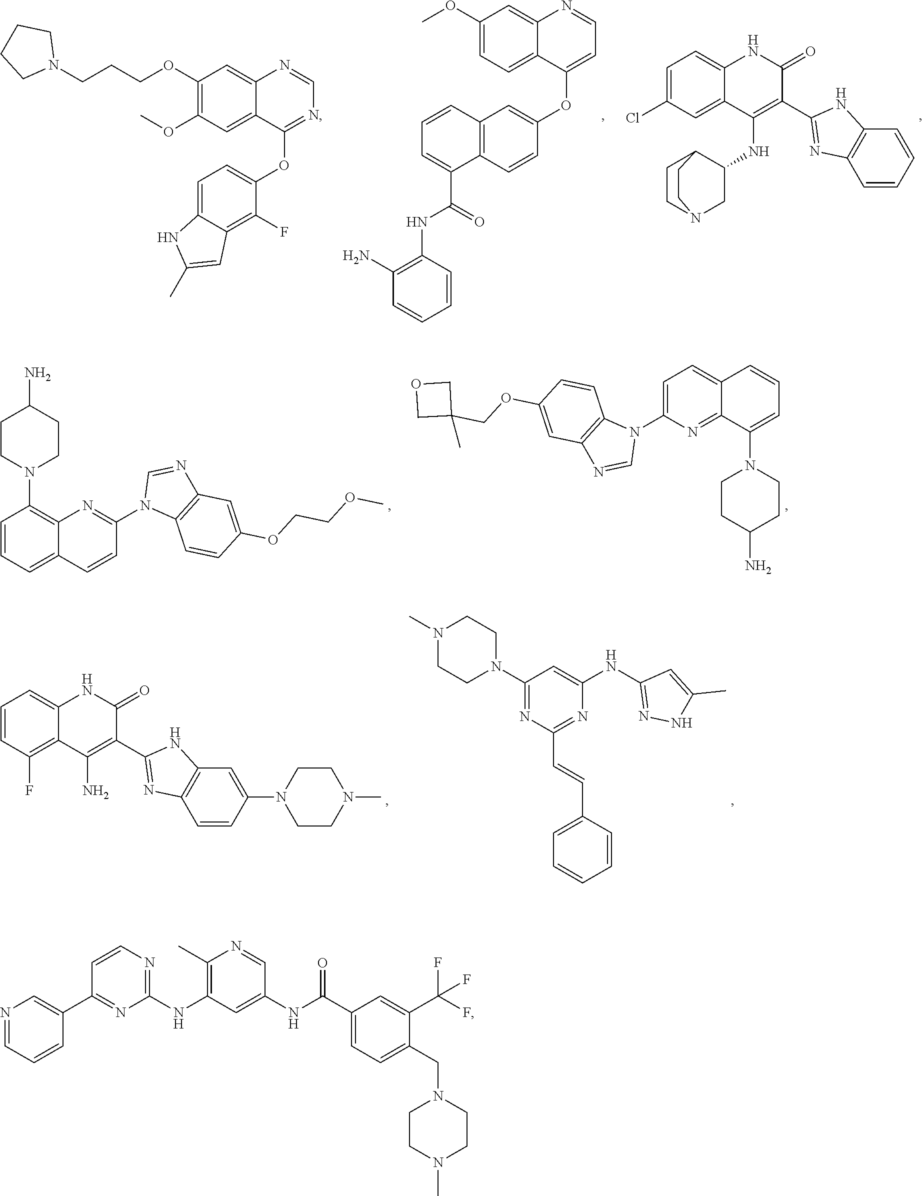

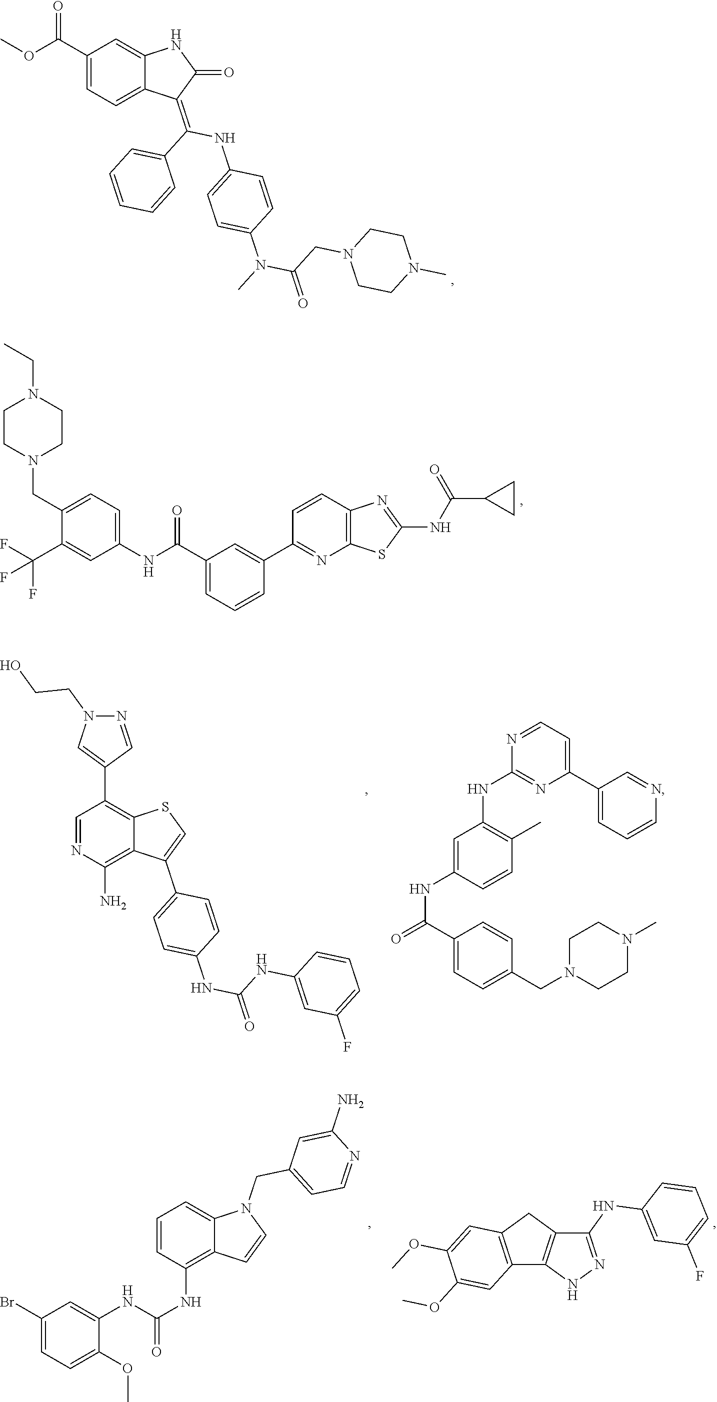

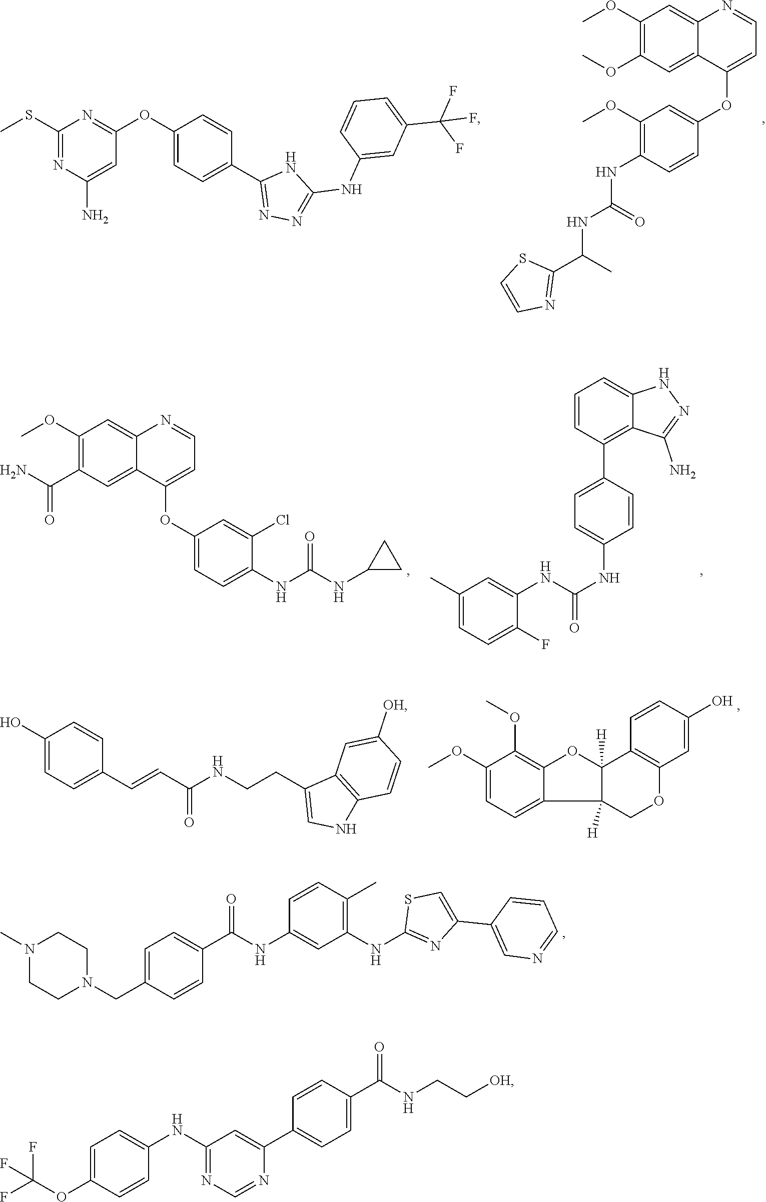

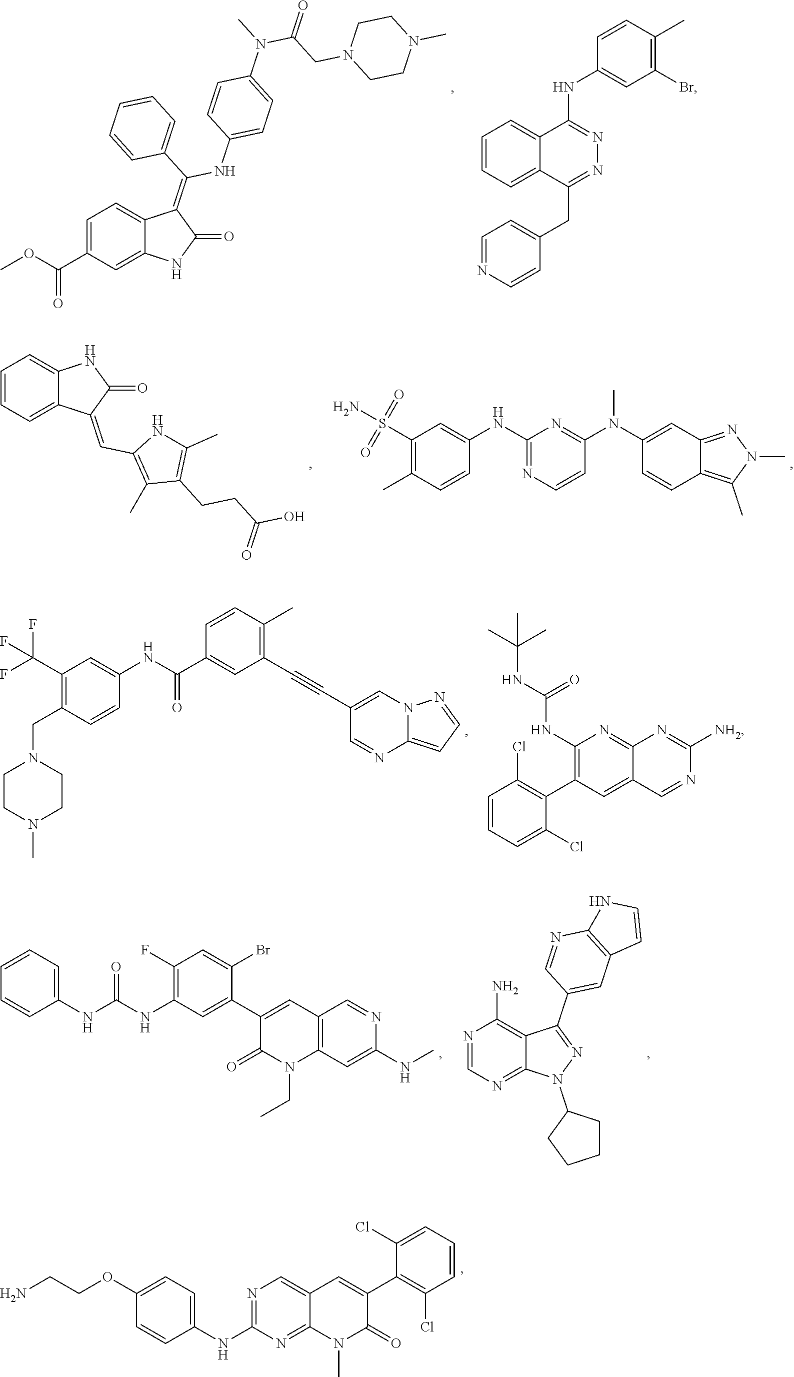

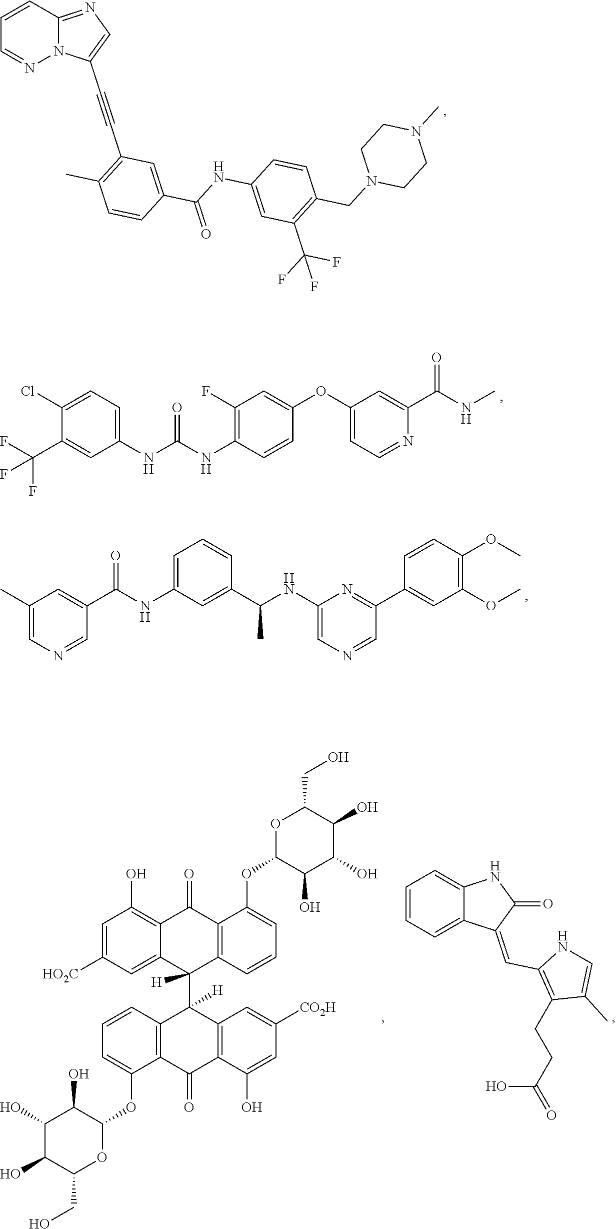

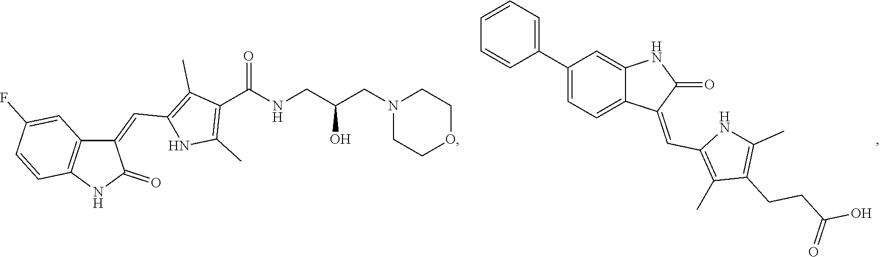 [SMILES: Cc1[nH]c(/C=C2\C(=O)Nc3ccc(F)cc32)c(C)c1C(=O)NC[C@H](O)CN1CCOCC1 ; Cc1[nH]c(/C=C2\C(=O)Nc3cc(-c4ccccc4)ccc32)c(C)c1CCC(=O)O]

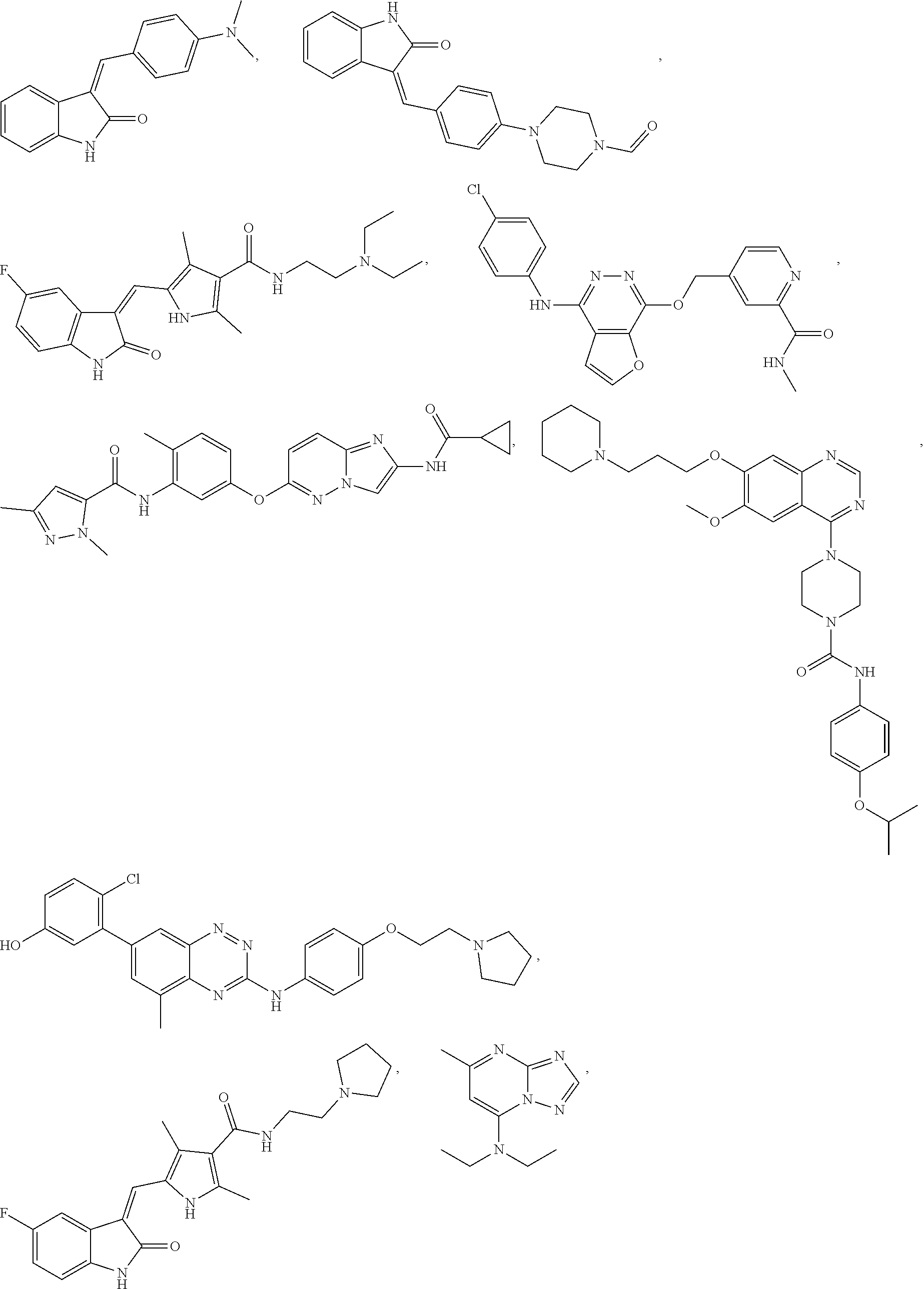

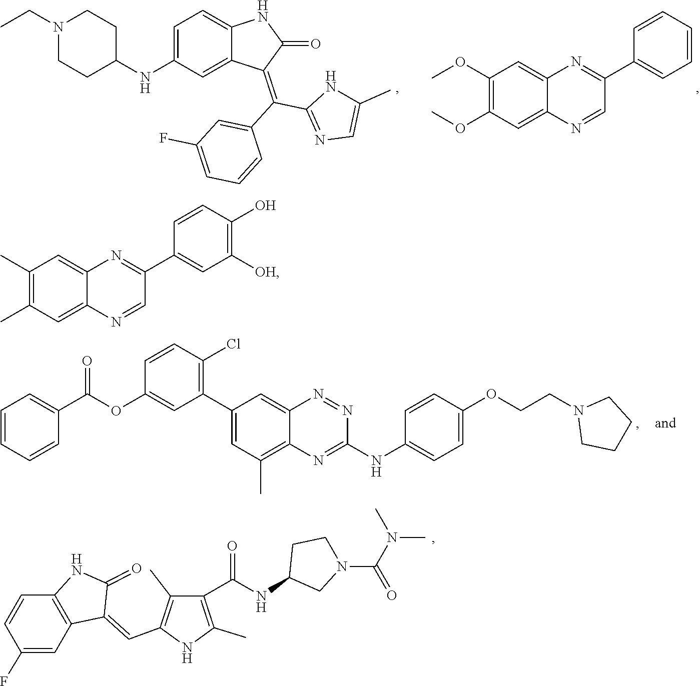

or pharmaceutically acceptable salts thereof.

In some embodiments, the PDGFR inhibitor is a hydrate.

In some embodiments, the PDGFR inhibitor is selected from:

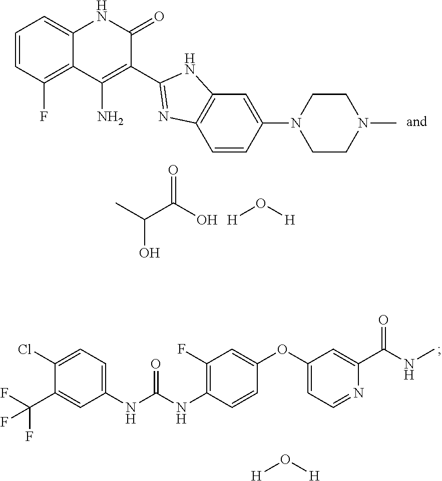

or pharmaceutically acceptable salts thereof In some embodiments, the PDGFR inhibitor is an isotope.

In some embodiments, the PDGFR inhibitor is deuterated.

In some embodiments, the PDGFR inhibitor is selected from:

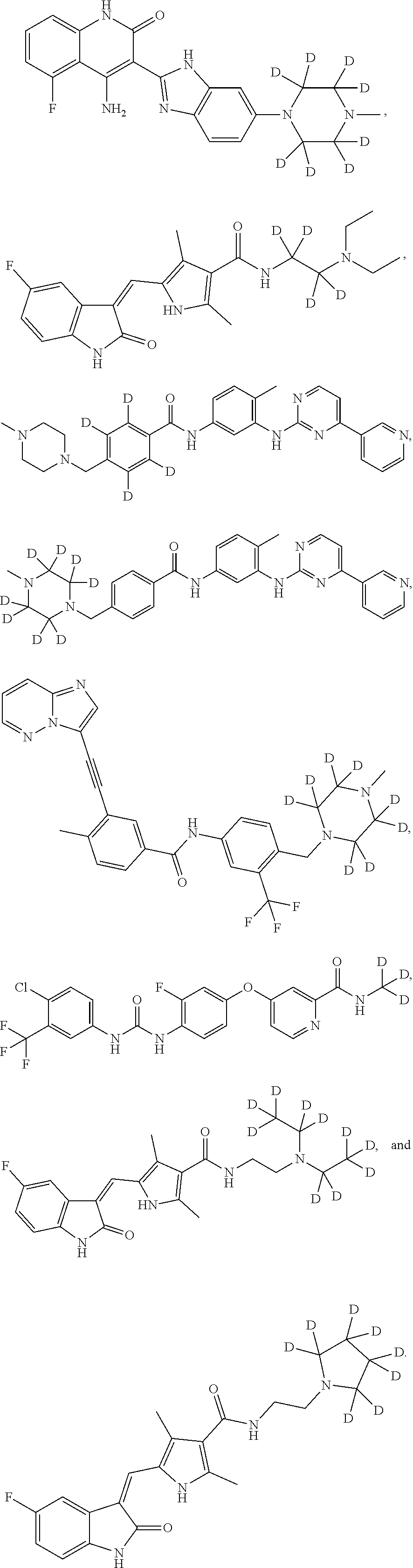

In some embodiments, the PDGFR inhibitor is exposed to the stem cell or neuronal cell as a pharmaceutically acceptable salt. In some embodiments, the PDGFR inhibitor is exposed to the crestosphere as a pharmaceutically acceptable salt.

In some embodiments, the pharmaceutically acceptable salt is a mesylate, a hydrochloride, a maleate, a lactate, a tartrate, a formate, an esylate, a phosphate, or a malate.

In some embodiments, the pharmaceutically acceptable salt has a structure selected from:

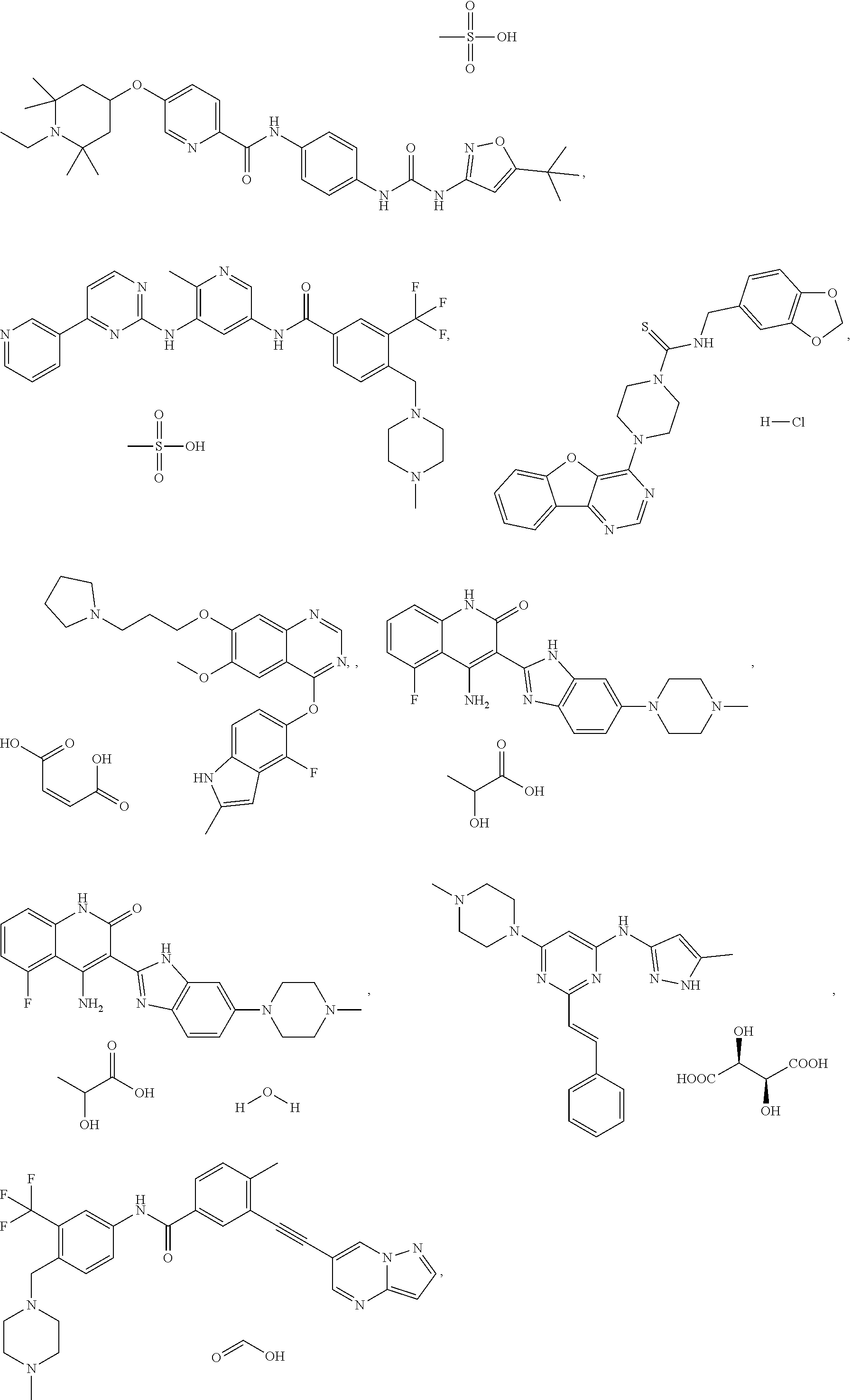

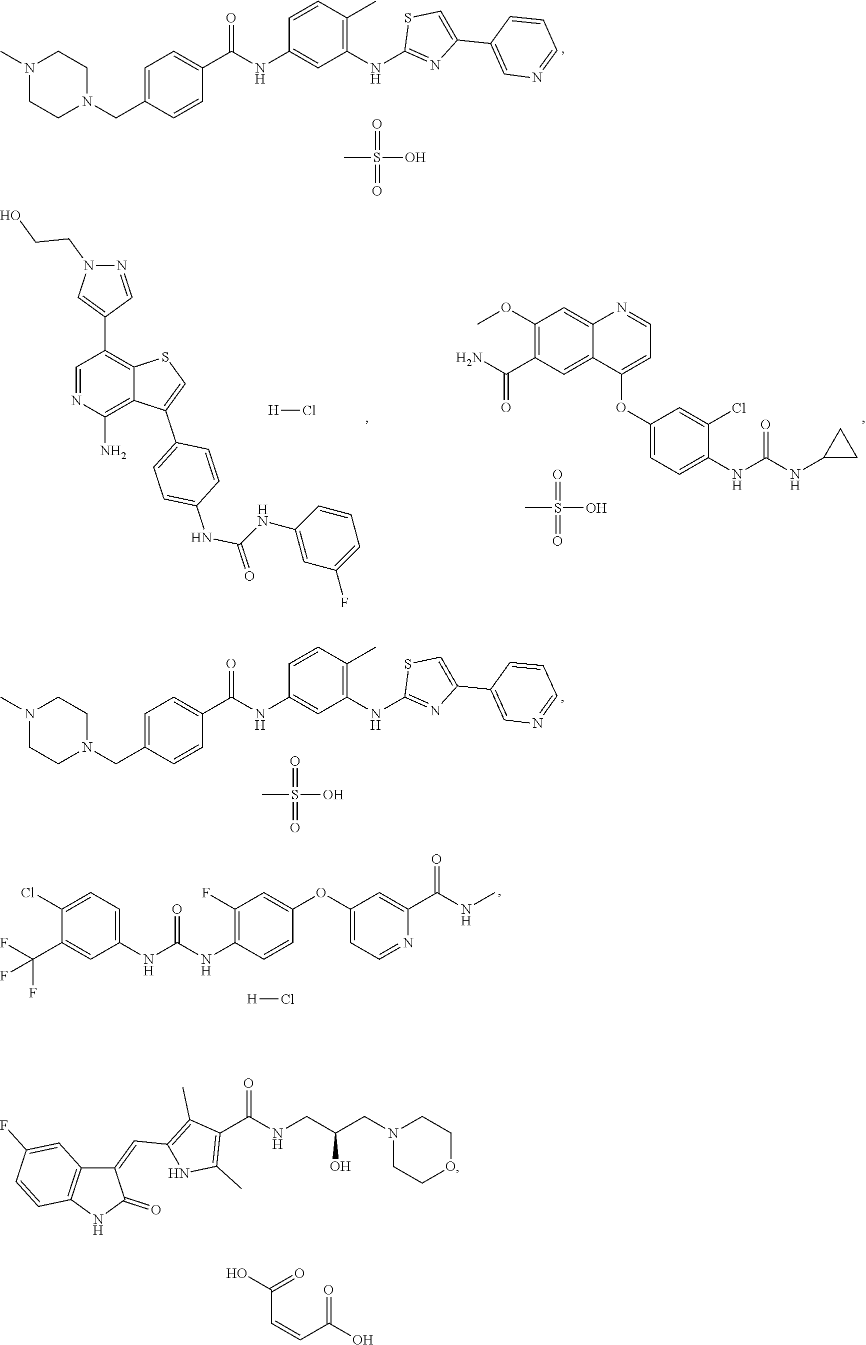

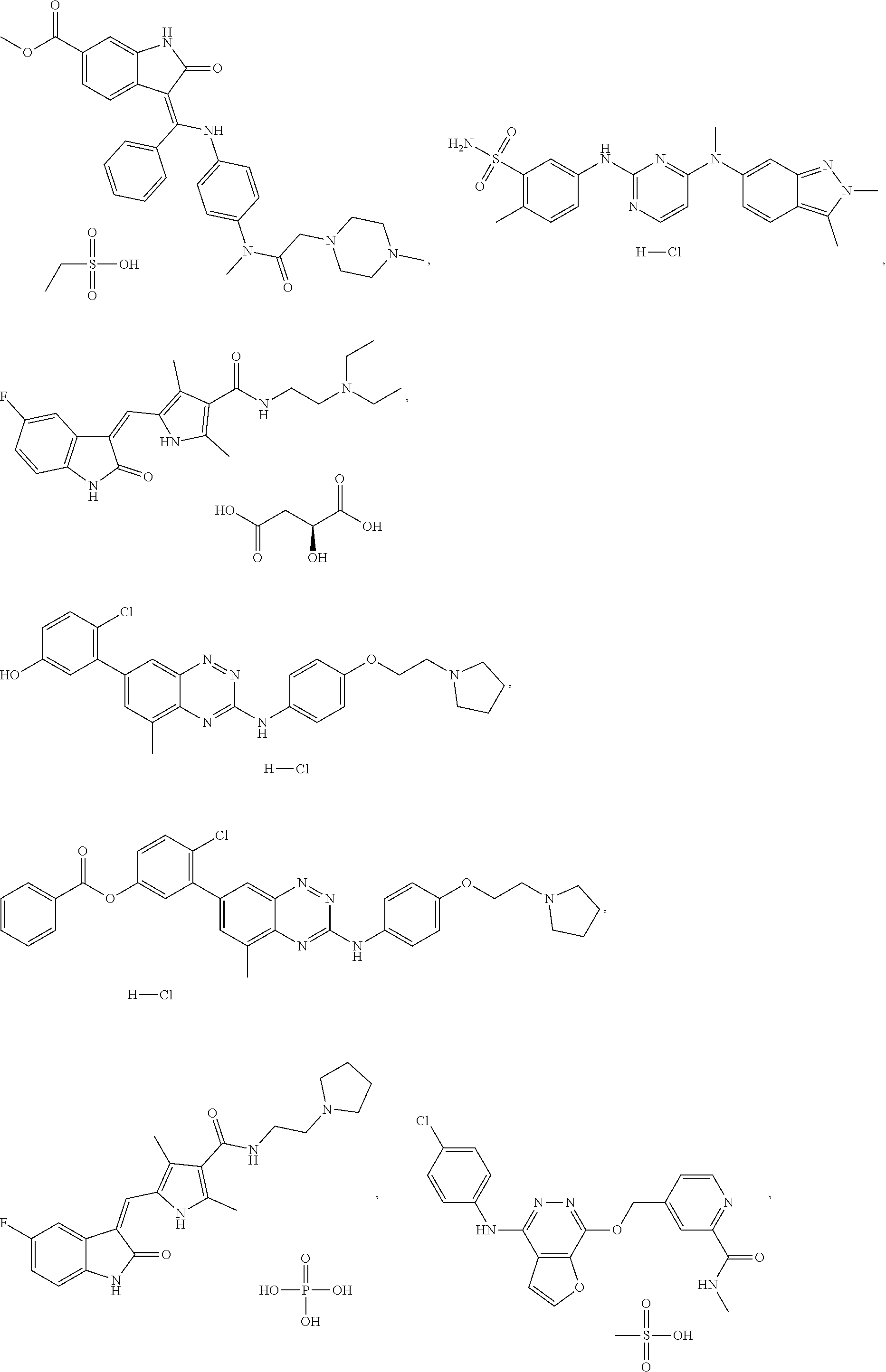

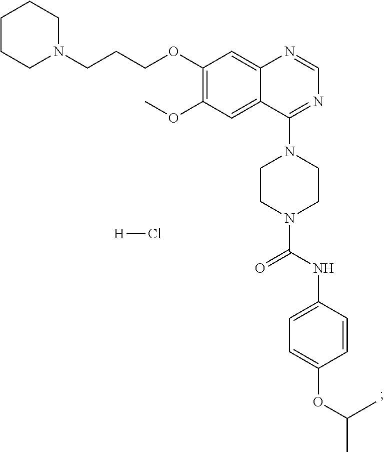 [SMILES: COc1cc2c(N3CCN(C(=O)Nc4ccc(OC(C)C)cc4)CC3)ncnc2cc1OCCCN1CCCCC1 ; Cl]

or a pharmaceutically acceptable salt thereof.

In another aspect, the disclosure relates to a method for modulating NO neuronal activity in a cell culture, the method comprising exposing cells in cell culture with an effective amount of a PDGFR inhibitor or a pharmaceutically acceptable salt thereof for a time period sufficient for the cell to differentiate into an enteric neuron.

In some embodiments, the cell culture comprises two-dimensional or three-dimensional neural crest cells.

In some embodiments, the modulating NO neuron activity is nitric oxide responsiveness.

In another aspect, the disclosure relates to a method of enriching NO enteric neurons in a cell culture comprising exposing a composition of neural crest cells or a crestosphere with an effective amount of a PDGFR inhibitor or a pharmaceutically acceptable salt thereof for a time period sufficient for the cell to differentiate into an enteric neuron.

In some embodiments, the cell culture comprises two-dimensional or three-dimensional neural crest cells. In some embodiments, the modulating NO neuron activity is nitric oxide responsiveness.

In another aspect, the disclosure relates to a kit comprising a PDGFR inhibitor, or a pharmaceutically acceptable salt thereof, and one or more selected from: a) instructions for treating a gut motility disorder; and b) instructions for administering the compound in connection with treating a gut motility disorder.

In some embodiments, the kit comprises a cell line comprising neural crest cells or a crestosphere. In some embodiments, the kit comprises a stem cell or a differentiated human stem cell. In some embodiments, the kit further comprises one or a plurality of enteric neurons. In some embodiments, the agent is selected from a parasympathomimetic, a prokinetic agent, an opioid antagonist, an antidiarrheal, and an antibiotic.

In some embodiments, the agent is selected from neostigmine, bethanechol, metoclopramide, cisapride, and loperamide. In some embodiments, the PDGFR and a cell line or cell culture are co-packaged. In another aspect, the present disclosure relates to a composition comprising one or a plurality of enteric glial cell, wherein the enteric glial cells comprise SOX10 and PMP22. In another aspect, the present disclosure relates to a composition comprising one or a plurality of enteric glial cell, wherein the enteric glial cells comprise PMP22.

In another aspect, the present disclosure relates to a composition comprising a spheroid comprising one or a plurality of enteric glial cells, wherein the enteric glial cells comprise or express SOX10 and PMP22. In some embodiments, the enteric glial cells further comprise SB100. In some embodiments, the enteric glial cells further comprise PLP1. In some embodiments, the enteric glial cells further comprise AQP4. In some embodiments, the enteric glial cells further comprise GFAP. In some embodiments, the enteric glial cells further comprise MPZ. In some embodiments, the enteric glial cells further comprise MBP. In some embodiments, the one or plurality of enteric glial cells are from an induced pluripotent stem cell. In some embodiments, the one or plurality of enteric glial cells are from a human induced pluripotent stem cell.

In some embodiments, the one or plurality of enteric glial cells are present in a ganglioid or a spheroid or a substantially spherical composition of cells.

In some embodiments, the one or plurality of enteric glial cells are in cell culture for no less than about 5, 10, 12, or 15 or more days.

In some embodiments, the one or a plurality of enteric glial cells and one or a plurality of enteric neuronal cells, wherein the enteric glial cells comprise SOX10 and PMP22; and wherein the enteric neuronal cells comprise SOX10 and at least one or a combination of: CD24, CD45RA, CD57, CD63, CD71, CD121b, CD147, CD164, CD184, CD193, CD243, CD275.

In another aspect, the disclosure relates to a composition comprising one or a plurality of enteric neuronal cells and (i) one or a plurality of mesenchymal cells; and (ii) one or a plurality of epithelial cells; and wherein the cells are positioned within a spheroid or ganglioid.

In some embodiments, the composition further comprises one or a plurality of smooth muscle cells; and/or enteric glial cells.

In some embodiments, the mesenchymal cells express one or a combination of: PRRX1, RNX2, TWIST1, COL11A1, COL1A2, COL1A1, COL3A1, COL5A2, FN1, LAMA4, EDNRA, PDGFRA, and PDGFRB. In some embodiments, the epithelial cells express one or a combination of: GALR1, CFC1, AC073941.1, TTC6, ARX, AC012405.1, CMTM8, SHH, PLSCR5, and CNTN4-AS2. In some embodiments, the enteric neurons express one or a combination of: NRXN3, NRXN1, DCX, MAPT, ELAVL2, NRCAM, RBFOX3, NCAM1, NRG1, SYN1, and SYP.

In some embodiments, the enteric neuronal cells comprise SOX10 and at least one or a combination of CD24, CD45RA, CD57, CD63, CD71, CD121b, CD147, CD164, CD184, CD193, CD243, and CD275.

In some embodiments, the composition further comprises one or a plurality of progenitor or stem-like cells expressing one or the combination of biomarkers in FIG. 8F.

In some embodiments, the glial cells express one or a combination of GFAP, ERBB4, NTRK2, NTRK3, PAX3, EDNRB, FZD3, and SOX2.

In some embodiments, the epithelial cells express one or a combination of: CDH1, EPCAM, and KRT119. In some embodiments, the smooth muscle cells comprise ACTA1, PAX7, MYOD1, MYL4, CHRNA1, TNNT2, MYOG, DES, and TBX1.

In some embodiments, the composition further comprises RPE cells expressing one or the combination of biomarkers in FIG. 8H.

In some embodiments, the glial cells express one or a combination of: GFAP, ERBB4, NTRK2, NTRK3, PAX3, EDNRB, FZD3, and SOX2; wherein the epithelial cells express one or a combination of: CDH1, EPCAM, KRT119; wherein the enteric neurons express one or a combination of: NRXN3, NRXN1, DCX, MAPT, ELAVL2, NRCAM, RBFOX3, NCAM1, NRG1, SYN1, and SYP; and wherein the mesenchymal cells express one or a combination of: PRRX1, RNX2, TWIST1, COL11A1, COL1A2, COL1A1, COL3A1, COL5A2, FN1, LAMA4, EDNRA, PDGFRA, and PDGFRB.

In some embodiments, the mesenchymal cells express KRT119 and one or a combination of: PRRX1, RNX2, TWIST1, COL11A1, COL1A2, COL1A1, COL3A1, COL5A2, FN1, LAMA4, EDNRA, PDGFRA, and PDGFRB.

In some embodiments, the composition is free of or substantially free of retinal pigment epithelium (RPE) cells.

In another aspect, the present disclosure relates to a method of enriching cells of a crestosphere or spheroid by exposing the crestosphere with a PDFGR inhibitor or a pharmaceutically acceptable salt thereof and one or a combination of: GDNF, ascorbic acid, neurobasal, n2 and b27.

In some embodiments, the PDGFR inhibitor is not PP1121, or is free or substantially free of PP1121 or a pharmaceutically acceptable salt thereof.

In another aspect, the present disclosure relates to a method of transplanting a spheroid of cells into a subject by administering the spheroid of cells into the gastrointestinal tract of the subject.

In some embodiments, the spheroid comprises the composition of any of claims 50 through 72.

In some embodiments, the subject has or is suspected of having a gut motility disorder.

In some embodiments, the gut motility disorder is selected from achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

In some embodiments, the step of administering comprises seeding the cells into the small intestine, stomach or colon of the subject.

In another aspect, the present disclosure relates to a method of treating a gut motility disorder in a subject in need thereof, the method comprising administering to the subject an effective amount of the composition of any of the enteric neurons or glial cell disclosed herein.

In some embodiments, the gut motility disorder is selected from achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

In another aspect, the present disclosure relates to a subject comprising any one of the compositions of any of the enteric neurons or glial cells disclosed herein. In some embodiments, the subject is a mouse or human. In some embodiments, the mouse is NOS double knockout (NOS−/−).

The present disclosure also relates to a cell line comprising the enteric neuronal cell disclosed herein. In another aspect, the present disclosure relates to a cell line comprising the enteric glial cell disclosed herein.

### BRIEF DESCRIPTION OF THE DRAWINGS

FIG. 1A. Protocol schematic for in vitro differentiation and maturation of hPSCs into enteric neural crest and enteric crestospheres.

FIG. 1B. scRNA-seq UMAP of cell types present in enteric neural crest cells (D10, top panel) and enteric crestosphere cells (D15, bottom panel) of the differentiation cultures depicted in FIG. 1A.

FIG. 1C. UMAP of enteric neural crest (D10, top) and enteric crestosphere (D15, bottom) subtypes in differentiation cultures.

FIG. 1D. Violin plot stack showing the expression of canonical enteric neural crest markers in enteric neural crest (top) and enteric crestosphere (bottom) subtypes.

FIG. 1E. Protocol schematic for in vitro differentiation and maturation of hPSC-derived enteric crestospheres into 2D ENS cultures and 3D ganglioids.

FIG. 1F. snRNA-seq UMAP of cell types present in stage 1 enteric ganglioids.

FIG. 1G. snRNA-seq UMAP of cell types present in stage 2 enteric ganglioids.

FIG. 1H. Immunofluorescence analysis for expression of neuronal TUBB3 and glial GFAP in stage 1 and stage 2 enteric ganglioids.

FIG. 1I. Immunofluorescence analysis for expression of neuronal activity marker cFOS in stage 1 and stage 2 enteric ganglioids.

FIG. 1J. Flow cytometry quantification of neuronal activity marker cFOS in enteric ganglioids as they mature.

FIG. 1K. Live fluorescence images of human hSYN-ChR2-EYFP in enteric ganglioids as they mature.

FIG. 1L. Quantification of multi-electrode array (MEA) analysis of baseline and blue light-stimulated neuronal activity in stage 1 hSYN-ChR2-EYFP (left) and control (right) enteric ganglioids.

FIG. 1M. Dot plot of the average module scores of stage 1 enteric ganglioid cell type transcriptional signatures in stage 2 enteric ganglioid cell types.

FIG. 1N. Projection of stage 2 cell types (right) onto the SWNE of stage 1 enteric ganglioid cells with overlayed projection of stage 1 cell-type specific transcription factors from FIG. 13.

FIG. 2A. snRNA-seq UMAP of neuronal subtypes present in stage 1 enteric ganglioids.

FIG. 2B. snRNA-seq UMAP of neuronal subtypes present in stage 2 enteric ganglioids.

FIG. 2C. Projection of stage 2 neuronal subtypes (right) onto the SWNE of stage 1 enteric ganglioid neurons with overlayed projection rate-limiting neurotransmitter synthesis enzymes.

FIG. 2D. Dot plot of the average module scores of stage 1 (bottom) and stage 2 (top) ganglioid cell type transcriptional signatures adult human colon cell types.

FIG. 2E. Dot plot of the average module scores of stage 1 (bottom) and stage 2 (top) ganglioid neuronal subtype transcriptional signatures adult human enteric neuron subtypes.

FIG. 2F. Immunofluorescence analysis for expression of ENS cell-type markers (serotonin, CHAT, GABA and NOS1) in stage 1 enteric ganglioids.

FIG. 2G. Quantification of flow cytometry analysis for the expression of neuronal subtype markers serotonin, CHAT, GABA and NOS1 in stage 1 2D ENS cultures (left) and 3D enteric ganglioids (right).

FIG. 2H. Flow cytometry validation of stage 1 EN 8 surface markers CCR6 (left) and GYPB (right) co-labeling with neurochemical markers showing enrichment of neurochemical identities of marker positive populations normalized to baseline neurochemical population levels.

FIG. 2I. Overall percentage of neurotransmitter synthesizing neurons in stage 1 and 2 enteric ganglioids compared to mouse and human primary enteric neurons.

FIG. 2J. Schematic of mono- and multi-neurotransmitter synthesis in enteric neurons.

FIG. 2K. Percentage of neurons showing mono- and multi-neurotransmitter profiles in stage 1 and 2 enteric ganglioid neurons compared to mouse and human primary enteric neurons.

FIG. 2L. Immunostaining of primary human colon with antibodies against NOS1, GABA and TUBB3 (top), and CHAT, GABA and TUBB3 (bottom). White dash line indicates the border of TUBB3⁺ ganglia. White arrows indicate colocalization.

FIG. 2M. Percentage of mono-neurotransmitter (top) and bi-neurotransmitter (bottom) producing enteric neurons in stage 1, 2 enteric ganglioids and primary datasets.

FIG. 3A. Schematic of snRNA-seq analysis and subsequent glial subclustering of stage 2 enteric ganglioids.

FIG. 3B. UMAP of glial subtypes present in stage 2 enteric ganglioid (snRNA-seq, left) and distribution of glial subtypes in biological replicates of enteric ganglioid cultures (right).

FIG. 3C. UMAP of enteric glial subtypes present in a primary adult human dataset.

FIG. 3D. Distribution of enteric glial subtype representation in individual human tissue samples.

FIG. 3E. Violin stack plot of the expression of canonical glial markers in stage 2 enteric ganglioid and adult human glial subtypes.

FIG. 3F. Immunofluorescence staining of canonical glial markers GFAP and S100 in stage 2 enteric ganglioids and human primary colon tissue.

FIG. 3G. Co-staining of GFAP and S100 in stage 2 enteric ganglioids.

FIG. 3H. Dot plot of the average module scores of stage 2 enteric ganglioid glial subtype transcriptional signatures in adult human enteric glial subtypes.

FIG. 3I. Immunostaining of myelinating markers in human colon and enteric ganglioids. PMP22 expression in stage 2 enteric ganglioid (top), PMP22 (middle) and MPZ (bottom) expression in human colon.

FIG. 3J. Feature plots showing the module scores of stage 1 enteric ganglioid progenitor 1 (top) and 2 (bottom) transcriptional signatures in ganglioid glia cells.

FIG. 3K. Heatmap showing the normalized enrichment scores of GO pathways enriched in each glia class determined by hierarchical clustering.

FIG. 4A. Schematic of CD24′/NOS1:GFP⁺ FACS sorted neurons' bulk RNA-seq analysis (top) and snRNA-seq analysis and subsequent NO neuron subclustering of stage 1 enteric ganglioids (bottom).

FIG. 4B. snRNA-seq UMAP of NO subtypes present in stage 1 enteric ganglioid neurons.

FIG. 4C. snRNA-seq UMAP of subclustered NO neuron subtypes from stage 1 enteric ganglioids.

FIG. 4D. Violin plot of (top) NOS1 expression and (bottom) module scoring for nitric oxide biosynthesis gene ontology (GO) term genes by stage 1 enteric ganglioid NO subtypes.

FIG. 4E. UMAP of pNO subtypes present in adult human enteric neurons.

FIG. 4F. UMAP of subclustered pNO subtypes from adult human enteric neurons.

FIG. 4G. Dot plot of the average module scores of adult human pNO neuron subtype transcriptional signatures in stage 1 enteric ganglioid NO neuron subtypes (left), and stage 1 enteric ganglioid NO neuron subtype transcriptional signatures in adult human pNO neuron subtypes (right).

FIG. 4H. Heatmap matrix of Spearman correlations based on scaled expression of 3000 anchor features shared significantly variable genes (or anchor features) between adult human (x-axis) and stage 1 enteric ganglioid (y-axis) NO neuron subtypes.

FIG. 4I and FIG. 4J. Dot plot of the scaled average expression of NO neurons specific transcription factors (TF), neuropeptides (NP), neurotransmitter receptors (NT-R), neuropeptide receptors (NP-R), and surface markers (SM) in stage 1 enteric ganglioid (I) and adult human (J) NO neuron subtypes versus non-NO neurons.

FIG. 4K and FIG. 4L. Feature plots of predicted neurotransmitter producing neuron identities in stage 1 enteric ganglioid (K) and adult human (L) subclustered NO neurons.

FIG. 4M. Distribution of neurochemical identities in stage 1 enteric ganglioid (left) and adult human (right) NO neuron subtypes versus non-NO neurons.

FIG. 4N. Dot plot of the average module scores for myenteric and submucosal neuron transcriptional signatures in stage 1 enteric ganglioid (left) and adult human (right) NO neuron subtypes versus non-NO neurons.

FIG. 5A. Schematic representation of a high-throughput flow cytometry-based screening to identify compounds that induce cFOS expression in hESC-derived stage 2 enteric ganglioid NO neurons.

FIG. 5B. Target classes of the hits identified in enteric NO neuron cFOS induction screening (FIG. S20D, red dots). FIG. S20 data not shown, but it describes identifying enteric NO neuron modulators by functional high-throughput screenings. The data of S20 were disclosed in Majd et al., “hPSC-Derived Enteric Ganglioids Model Human ENS Development and Function”, bioRxiv, posted on Jan. 3, 2022, which is incorporated by reference in its entirety.

FIG. 5C. Schematic representation of a high-throughput calorimetry-based screening to identify compounds that induce NO release in hESC-derived stage 1 2D ENS cultures.

FIG. 5D. Target classes of the hits identified in NO release screening (Figure S20E, red dots). Protein classes that are in common with (B) are indicated with asterisks.

FIG. 5E. Feature plots showing the predicted responsiveness of subclustered stage 1 ganglioid enteric NO neurons to neurotransmitters by module scoring of neurotransmitter receptor gene families.

FIG. 5F. Dot plot of the expression of genes belonging to the target classes shown in B and D in hESC-derived stage 1 and primary human enteric nitrergic neuron subtypes versus all other neurons.

FIG. 5G. Combined protein target analysis for selected screening hits showing shared protein classes. Color code matches the target classes in (B) and (D).

FIG. 5H. Schematic representation of testing the effect of selected candidate hits (listed in (G)) on mouse colonic motility ex vivo.

FIG. 5I. Representative spatiotemporal map of mouse colon contractions along the proximal-distal axis over a 10-min period.

FIG. 5J. Quantification of colonic migrating motor complexes (CMMC) intervals at 75th percentile of CMMC cumulative percentage (data not shown) for selected hit compounds.

FIG. 5K. Experimental design for measuring the effect of selected candidate hits on mouse colonic motility ex vivo. Representative spatiotemporal maps of a mouse colon contraction along the proximal-distal axis over a 26-min period. Three representative longitudinal contractile events (LCEs) are shown per condition (arrows).

FIG. 5L. Diagrams of CMMC cumulative percentile and quantification of CMMC interval (time difference between two consecutive contractions) at 75th percentile for dexmedetomidine. Mean and SEM error bars for 5 pairs of untreated and drug-treated mouse colons are shown.

FIG. 5M. Total number of colonic longitudinal contractile events (LCEs) within each 6-min treatment condition for 5 dexmedetomidine-treated mouse colons measured from spatiotemporal maps. *: p-value <0.05.

FIG. 5N. Mean of LCE duration calculated for three LCEs within each 6-min treatment (1 in the beginning, 1 in the middle, and 1 in the end of each spatiotemporal map, see (K)). Data are shown for 5 dexmedetomidine-treated mouse colons. SEM error bars are shown.

FIG. 6A. Schematic representation of a high-throughput pharmacological screening to identify compounds that enrich NO neurons in hESC-derived 2D ENS cultures.

FIG. 6B. Combined protein target analysis for the HTS top 12 hits showing shared protein classes between structurally similar hits.

FIG. 6C. Effect of PP121 treatment window on NOS1::GFP induction efficiency.

FIG. 6D. Immunofluorescence staining of NOS1 and neuronal TUBB3 in stage 1 enteric ganglioids treated with or without PP121 between days 15 and 20.

FIG. 6E. Split UMAP of cell types present in stage 1 control (top) and PP121 treated (bottom) enteric ganglioid cultures.

FIG. 6F. Dot plot of the average module scores of control only enteric ganglioid subtype transcriptional signatures in PP121 treated ganglioid subtypes.

FIG. 6G. Split UMAP of neuronal subtypes present in stage 1 control (top) and PP121 treated (bottom) enteric ganglioid cultures.

FIG. 6H. Dot plot of the average module scores of control only neuronal subtype transcriptional signatures in PP121 treated ganglioid neuronal subtypes.

FIG. 6I. Distribution of NO neuron subtypes in control versus PP121 treated stage 1 enteric ganglioid cultures.

FIG. 6J. Split UMAP of subclustered NO subtypes present in stage 1 control (top) and PP121 treated (bottom) enteric ganglioid cultures.

FIG. 6K. Dot plot of the average module scores of control only NO neuron subtype transcriptional signatures in PP121 treated ganglioid NO neuron subtypes.

FIG. 6L. Feature plot showing the expression of ERBBs, PDGFRs and VEGFRs in D15 subclustered enteric crestospheres.

FIG. 6M. Schematic of receptor tyrosine kinase (RTK) natural agonists and selected pharmacological antagonists including NO neuron enriching top hit PP121.

FIG. 6N. Effect of RTK ligand treatment on stage 1 enteric ganglioid NO neuron induction.

FIG. 6O and FIG. 6P. Effect of knocking out PDGFRA (0) and PDGFRB (P) in D15 enteric crestospheres on stage 1 enteric ganglioid NO neuron enrichment as measured by flow cytometry.

FIG. 7A. Schematic showing transplantation of hESC-derived stage 1 enteric ganglioids into mouse proximal colon.

FIG. 7B. Engraftment of hESC-derived stage 1 enteric ganglioid cells into the entire length of mouse colon as shown by the expression of human cytoplasmic marker SC121 in red.

FIG. 7C. Immunohistochemical analysis of human cytoplasmic protein SC121, and NO neuron marker NOS1 in Nos1⁻/− mouse colon 8 weeks post transplantation.

FIG. 8A. Dot plot of the scaled average expression of cell type annotation genes for enteric neural crest (left) and enteric crestosphere (right) cell types. All data are derived from scRNA-seq analysis.

FIG. 8B. Dot plot of the average module scores of enteric neural crest cell type transcriptional signatures in enteric crestosphere cell types. All data are derived from scRNA-seq analysis.

FIG. 8C. Dot plot of the average module scores of enteric neural crest cells (D10) subtype transcriptional signatures in enteric crestosphere (D15) subtypes.

FIGS. 8D, 8E and 8F. Dot plot of the scaled average expression of the top 10 differentially expressed genes for each enteric neural crest (D10, D), enteric crestosphere (D15, E), stage 1 enteric ganglioid (F) cell type (Unknown clusters showing top 10 differentially expressed genes).

FIG. 8G. Dot plot of the average module scores of enteric crestosphere (D15) subtype transcriptional signatures in stage 1 enteric ganglioid cell types.

FIG. 8H. Dot plot of the scaled average expression of the top 10 differentially expressed genes for stage 2 enteric ganglioid cell types.

FIG. 8I. UMAP of epithelial and mesenchymal subtypes present in stage 2 enteric ganglioid cultures.

FIG. 8J. Dot plot of the scaled average expression of the 10 ten differentially expressed genes of stage 2 enteric ganglioid epithelial and mesenchymal subtypes.

FIG. 9A. Representative diagram of a spontaneous neuronal firing recorded during multi-electrode array analysis (MEA) of stage 1 enteric ganglioids.

FIG. 9B. MEA analysis of baseline and blue light-stimulated neuronal activities in stage 1 hSYN-ChR2-EYFP (left) and control (right) enteric ganglioids.

FIG. 9C. Flow cytometry analysis of cFOS expression in hSYN-ChR2-EYFP-derived stage 2 enteric ganglioids in response to blue light stimulation.

FIG. 10A through FIG. 10H. Violin plot stack of cell-type specific transcription factors (A), neurotransmitter receptors (B), neuropeptide receptors (C), cytokines (D), cytokine receptors (E), secreted ligands (F), ligand receptors (G), surface markers (H), in stage 1 enteric ganglioids.

FIG. 10I through FIG. 10P. Violin plot stack of cell-type specific transcription factors (I), neurotransmitter receptors (J), neuropeptide receptors (K), cytokines (L), cytokine receptors (M), secreted ligands (N), ligand receptors (O) and surface markers (P) in stage 2 enteric ganglioids.

FIG. 11A through FIG. 11H. Dot plot of the scaled average expression of selected transcription factor families (A), neurotransmitter receptors (B), neuropeptide receptors (C), cytokines (D), cytokine receptors (E), selected secreted ligands (F), selected ligand receptors (G) and surface markers (H) in stage 1 enteric ganglioid cell types.

FIG. 11A through FIG. 11H. Dot plot of the scaled average expression of selected transcription factor families (A), neurotransmitter receptors (B), neuropeptide receptors (C), cytokines (D), cytokine receptors (E), selected secreted ligands (F), selected ligand receptors (G) and surface markers (H) in stage 2 enteric ganglioid cell types.

FIG. 12A. Dot plot of the average module scores of stage 1 enteric ganglioid neuronal subtype transcriptional signatures in stage 2 enteric ganglioid neuronal subtypes.

FIG. 12B. UMAP of stage 2 2D ENS culture neuronal subtypes.

FIG. 12C. Dot plot of the scaled average expression of the top 10 differentially expressed genes for each stage 2 2D ENS culture neuron subtype.

FIG. 12D. Dot plot of the average module scores of stage 2 enteric ganglioid neuron subtype transcriptional signatures in stage 2 2D ENS culture neuron subtypes.

FIG. 12E. Comparison of the distribution of enteric neuron subtypes in 2D versus 3D enteric neuron cultures.

FIG. 12F. Heatmap matrix of Spearman correlations based on scaled expression of 3000 anchor features shared significantly variable genes (or anchor features) between stage 2 2D ENS cultures (x-axis) and ganglioids (y-axis).

FIG. 12G. UMAPs of cell types (top) and neuronal subtypes (bottom) present in a primary adult human colon dataset.

FIG. 12H. Heatmap matrix of Spearman correlations based on scaled expression of 3000 anchor features shared significantly variable genes (or anchor features) between stage 1 and 2 ganglioid neuron subtypes, and adult human enteric neuron subtypes.

FIG. 12I. Dot plot of the average module scores for myenteric and submucosal neuron transcriptional signatures in adult human enteric neuron subtypes (left) and stage 1 (middle) and stage 2 (right) ganglioid neuronal subtypes.

FIG. 13A and FIG. 13B. Feature plots showing the expression of rate limiting enzymes in neurotransmitter synthesis pathways by stage 1 (A) and stage 2 (B) enteric ganglioid neurons.

FIG. 13C and FIG. 13D. Feature plots showing the identity score of neurotransmitters by stage 1 (C) and stage 2 (D) enteric ganglioid neurons by module scoring of genes related to each neurotransmitter's synthesis, metabolism and reuptake.

FIG. 13E and FIG. 13F. UMAP of predicted neurotransmitter producing neuron identities in stage 1 (E) and stage 2 (F) enteric ganglioids.

FIG. 13G. Distribution of neurochemical identities in stage 1 (left) and stage 2 (right) enteric ganglioid neuron subtypes.

FIG. 13H. Distribution of neurochemical identities in stage 2 2D culture enteric neuron subtypes.

FIG. 13I. Comparison of the distribution of neurochemical identities in 2D culture versus 3D culture enteric neurons.

FIG. 13J and FIG. 13K. Feature plots showing the predicted responsiveness of stage 1 (J) and stage 2 (K) enteric ganglioid neurons to each neurotransmitter by module scoring of neurotransmitter receptor gene families.

FIG. 14A. Distribution of neurochemical identities in adult human (left) and adult mouse (right) enteric neuron subtypes.

FIG. 14B. Distribution of neurochemical identities in E15 (left), E18 (middle), and P21 (right) mouse enteric neuron subtypes.

FIG. 15A. scRNA-seq UMAP (left) and distribution of glial subtypes in biological replicates (right) in stage 2 2D ENS cultures.

FIG. 15B. UMAP of enteric glial subtypes present in a primary adult mouse dataset.

FIG. 15C. UMAP of enteric glial subtypes present in a P21 (left) and enteric glia and progenitor subtypes present in an E18 (right) adult mouse dataset.

FIG. 15D. Dot plot of the scaled average expression of the top 10 differentially expressed genes for each enteric ganglioid (top left), 2D ENS culture (top right), adult human (middle left), adult mouse (middle right), P21 mouse (bottom left) enteric glial subtypes and E18 mouse (bottom right) enteric glial and progenitor subtypes.

FIG. 15E. Violin plot stack showing the expression of canonical glial markers in 2D ENS culture, adult mouse, and P21 and E18 mouse glial (and progenitor) subtypes.

FIG. 15F. Dot plot of the average module scores of stage 2 enteric ganglioid glial subtype transcriptional signatures (snRNA-seq) in 2D ENS culture glial subtypes (scRNA-seq).

FIG. 15G. Heatmap matrix of Spearman correlations based on scaled expression of 3000 anchor features shared significantly variable genes (or anchor features) between 2D ENS cultures (x-axis) and enteric ganglioids (y-axis).

FIG. 16A through FIG. 16H. Violin plot stack of cell-type specific transcription factors (A), neurotransmitter receptors (B), neuropeptide receptors (C), cytokines (D), cytokine receptors (E), selected secreted ligands (F), selected ligand receptors (G) and surface markers (H) in enteric ganglioid glial subtypes.

FIG. 17A through FIG. 17H. Dot plot of the scaled average expression of selected transcription factor families (A), neurotransmitter receptors (B), neuropeptide receptors (C), cytokines (D), cytokine receptors (E), selected secreted ligands (F), selected ligand receptors (G) and surface markers (H) in enteric ganglioid glial subtypes.

FIG. 18A. Hierarchical clustering of enteric ganglioid glial subtypes with primary human and mouse glial subtypes based on normalized enrichment scores of biological process gene ontology (GO) pathways.

FIG. 18B. Dot plot of the average module scores for myenteric and submucosal glial transcriptional signatures in primary human enteric glial subtypes.

FIG. 18C. Distribution of enteric glial subtype representation in primary human myenteric versus submucosal tissue samples.

FIG. 18D. Dot plot of the average module scores for myenteric and submucosal glial transcriptional signatures in stage 2 ganglioid glial subtypes.

FIG. 19A. Schematic representation of the NOS1::GFP reporter construct.

FIG. 19B. Representative immunofluorescence images of a stage 2 enteric ganglioid stained for GFP and NOS1.

FIG. 19C. Representative flow cytometry analysis for the expression of GFP and NOS1 in a NOS1::GFP-derived stage 1 enteric ganglioid.

FIG. 19D. Bulk RNA-seq top 50 differentially expressed transcripts in FACS sorted CD24′/NOS1::GFP⁺ cells relative to CD24⁺/NOS1::GFP⁻ cells. p-value <0.05, upregulated in red, downregulated in blue.

FIG. 19E. Distribution of NO neuron subtypes in biological replicates of stage 1 enteric ganglioid cultures.

FIG. 19F. Dot plot of the scaled average expression of the top 10 differentially expressed genes for each stage 1 enteric ganglioid NO neuron subtype.

FIG. 19G. snRNA-seq analysis violin plot of module scoring for the top 100 differentially expressed genes from CD24′/NOS1⁺ sorted neurons versus other neurons in stage 1 enteric ganglioid NO subtypes versus other neurons.

FIG. 20A through FIG. 20J. Bulk RNA-seq differentially expressed (Log 2FC, p-value <0.05) genes in NOS1::GFP⁺ neurons versus other neurons, and snRNA-seq dot plot of the average expression of selected transcription factor families (A), neurotransmitter synthesis genes (B) neurotransmitter receptors (C), neuropeptide receptors (D), neuropeptides (E), cytokines (F), cytokine receptors (G), selected secreted ligands (H), selected ligand receptors (I) and surface markers (J) in stage 1 enteric ganglioid NO neurons.

### DETAILED DESCRIPTION OF EMBODIMENTS

The disclosure relates to compositions comprising ganglioid cells, spheroids and crestospheres comprising one or a plurality of enteric neurons. The disclosure relates to compositions comprising ganglioid cells, spheroids and crestospheres comprising enteric glial cells. The disclosure further relates to methods of differentiating human pluripotent stem cells in to making any two dimensional or three-dimensional cultures comprising enteric neurons and/or glial cells. The disclosure further relates to methods of transplanting those compositions into subjects, in one case to produce animal model comprising enteric neurons and/or enteric glial cells disclosed here; and, in another case, to administer spheroids, treated crestospheres comprising enteric neurons and/or enteric glial cells to subjects for treatment of gut motility disorders. In some embodiments, the enteric neurons are NO responsive or more NO responsive than enteric neurons from human pluripotent stem cells not exposed to a PDGFR inhibitor. The disclosure relates to exposing a crestosphere from iPSCs to a physiologically effective amount of a PDGFR inhibitor for a time period sufficient to enrich the number of enteric neurons or enteric glial cells in culture.

### Definitions

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, NY 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “substantially free of” as used herein refers to a composition that only has trace or negligible amounts of the substance to which it refers. In some embodiments, substantially free means that the composition comprises only about 0.1%, 0.2%, 0.3% 0.4% or 0.5% of the substance to which it refers. In some embodiments, substantially free means that the composition comprises less than about 1.0% of the substance to which it refers relative to the number or mass of substances in the compositions and confers no biological effect to the compositions.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated glial cells. In some embodiments, the culture vessel comprises a hydrogel and one or a plurality of isolated glial cells, to which one or a plurality of neuronal cells are seeded.

The term “exposing” as used herein refers to bringing a disclosed compound and a cell, target receptor, or other biological entity together in direct or indirect contact, in such a manner that the compound can affect the activity of the cell (e.g., receptor, cell, etc.). Directly this can occur by physical contact between the disclosed compound and the cell, receptor o other entity; i.e., by interacting with the target or cell itself, or indirectly this can occur by interacting with another molecule, co-factor, factor, or protein on which the activity of the cell is dependent. In some embodiments, the activity of the cell in response to the compound or molecule is differentiation. In some embodiments, the compound is one or more differentiation factors.

“Analogues” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like. The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “progenitor cell” as used herein is defined as a cell that is pluripotent cell exposed to cell medium that comprises differentiation factors but remains pluripotent at least partially undiffrentiated. In some embodiments, a progenitor cell comprises WNT2B+. In some embodiments, a progenitor cell comprises PAX6′.

The term “pluripotent stem cell” as used herein is defined as a cell that is self-replicating capable of developing into cells and tissues of the three primary germ layers. Pluripotent stem cells include embryonic and induced pluripotent cells as defined herein. Contemplated pluripotent stem cells originate from mammals, e.g., human, mouse, rat, monkey, horse, goat, sheep, dog, cat etc.

The term “induced pluripotent stem cell” (iPSC) means a type of pluripotent cell made by reprogramming a somatic cell to have the same properties as embryonic stem cells, namely, the ability to self-renew and differentiate into the three primary germ layers. In some embodiments, iPSCs include mammalian cells, e.g., human, mouse, rat, monkey, horse, goat, sheep, dog, cat etc., reprogrammed to express Oct4, Nanog, Sox2, and optionally c-Myc. In some embodiments, iPSCs comprise reprogrammed primary cell lines. In some embodiments, iPSCs are obtained from a repository, such as the Coriell Institute for Medical Research (e.g., Catalog ID GM25256 (WTC-11), GM25430, GM23392, GM23396, GM24666, GM27177, GM24683), California Institute for Regenerative Medicine: California's Stem Cell Agency (e.g., CW60261, CW60354, CW60359, CW60480, CW60335, CW60280, CW60594, CW60083, CW60086, CW60087, CW60167, CW60186), and the American Type Culture Collection (ATCC®) (e.g., ATCC-DYR0530 Human Induced Pluripotent Stem (IPS) Cells (ATCC® ACS-1012™, ATCC® ACS-101 1™, ATCC® Number: ACS-1024™, ATCC® Number: ACS-1028™, ATCC® Number: ACS-1031™, ATCC® Number: ACS-1004™, ATCC® Number: ACS-1029™, ATCC® Number: ACS-1020™, ATCC® Number: ACS-1007™, ATCC® Number: ACS-1030™). Induced pluripotent stem cells may be derived from cell types such as fibroblasts taken from the skin, lung, or vein of subjects that are apparently healthy or diseased.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “embryonic stem cell line” as used herein is defined as a cell derived from the inner cell mass of the pre-implantation blastocyst capable of self-renewal and differentiation into the three primary germ layers. In some embodiments, embryonic stem cell lines listed in the NIH Human Embryonic Stem Cell Registry, e.g., CHB-1, CHB-2, CHB-3, CHB-4, CHB-5, CHB-6, CHB-8, CHB-9, CHB-10, CHB-11, CHB-12, RUES1, RUES2, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 26, HUES 27, HUES 28, CyT49, RUES3, WA01 (H1), UCSF4, NYUES1, NYUES2, NYUES3, NYUES4, NYUES5, NYUES6, NYUES7, MFS5, HUES 48, HUES 49, HUES 53, HUES 65, HUES 66, UCLA 1, UCLA 2, UCLA 3, WA07 (H7), WA09 (H9), WA13 (H13), WA14 (H14), HUES 62, HUES 63, HUES 64, CT1, CT2, CT3, CT4, MA135, Endeavour-2, WIBR1, WIBR2, HUES 45, Shef 3, Shef 6, WIBR3, WIBR4, WIBR5, WIBR6, BJNhem19, BJNhem20, SA001, SA002, UCLA 4, UCLA 5, UCLA 6, HUES PGD 13, HUES PGD 3, ESI-014, ESI-017, HUES PGD 11, HUES PGD 12, WA15, WA16, WA17, WA18, WA19, etc. In some embodiments, embryonic stem cells comprise gene(s) associated with diseases or disorders.

The term “enteric neural crest cell” means a cell produced by inducing differentiation of a pluripotent stem cell, wherein the enteric neural crest cell expresses SOX10, PHOX2B, EDNRB, TFAP2A, BRN3A, ISL1 and/or ASCL1. In some embodiments, the enteric neural crest cell comprises FOX3D. In some embodiments, the neural crest cell is present in an embryoid body or neural rosette. In some embodiments, the neural crest cell expresses vagal markers HOXB2, HOXB3, and/or HOXB5. In some embodiments, neural crest cells express p75 and HNK1. In some embodiments, neural crest cells express HOXB2, HOXB3, HAND2 and EDNRB.

The term “enteric neuron” means a cell that exhibits downregulation of SOX10, sustained expression of EDNRB, ASCL1 and PHOX2B, and upregulation of TUJ1 and TRKC. In some embodiments, enteric neurons express neuronal subtype specific markers including the cholinergic neuronal marker Choline Acetyl Transferase (CHAT), serotonin (5-HT) receptor, gamma-Aminobutyric acid (GABA), and neuronal nitric oxide synthase (nNOS). In some embodiments, CHAT expression indicates the presence of cholinergic neurons. In some embodiments, expression of NOS1 indicates the presence of nitrergic neurons. In some embodiments, enteric neurons include glial cells expressing glial fibrillary acidic protein (GFAP) and SOX10. In some embodiments, the enteric neuron is produced by inducing differentiation of an enteric neural crest cell. In some embodiments, the enteric neurons express SOX10, sustained expression of EDNRB, ASCL1 and PHOX2B, and upregulation of TUJ1 and TRKC.

The term “enteric glial cell” means a cell that exhibits expression of SOX10 and: GPAP and/or PMP22. In some embodiments, the enteric glial cell exhibits expression of SOX10 and PMP22. In some embodiments, the enteric glial cells is produced by inducing differentiation of an enteric neural crest cell.

The term “rho kinase inhibitor” means a compound that decreases the activity of rho kinase. In some embodiments, the rho kinase inhibitor is N-[(3-Hydroxyphenyl)methyl]-N′-[4-(4-pyridinyl)-2-thiazolyl]urea dihydrochloride (RKI-1447), (+)-(R)-trans-4-(1-aminoethyl)-N-(4-pyridyl)cyclohexanecarboxamide dihydrochloride (Y-27632), Fasudil (HA-1077), Hydroxyfasudil (HA 1100 hydrochloride), Thiazovivin, GSK429286A, Narciclasine, and/or (+)-(R)-trans4-(1-aminoethyl)-N-(1H-pyrrolo[2,3-b]pyridin-4-yl)cyclohexanecarboxamide dihydrochloride (Y-30141).

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof. In some embodiments, the hydrogel or hydrogel matrix comprises Matrigel® or vitronectin. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “Matrigel®” means a solubilized basement membrane preparation extracted from the Engelbreth-Holm-Swarm (EHS) mouse sarcoma comprising ECM proteins including laminin, collagen IV, heparin sulfate proteoglycans, entactin/nidogen, and other growth factors. In some embodiments, Cultrex® BME (Trevigen, Inc.) or Geltrex® (Thermo-Fisher Inc.) may be substituted for Matrigel®.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

The term “vitronectin” means a protein encoded by the VTN gene. In some embodiments, vitronectin has at least about 70%, 80%, 85%, 90%, 91%, 92%, 93%, 94%, 95%, 96%, 97%, 98%, 99% or 100% sequence identity with SEQ ID NO: 1, SEQ ID NO: 2, SEQ ID NO: 3, or a fragment thereof.

>sp|P04004|VTNC_HUMAN Vitronectin OS = Homo sapiens OX = 9606
GN = VTN PE = 1 SV = 1
SEQ ID NO: 1
MAPLRPLLILALLAWVALADQESCKGRCTEGFNVDKKCQCDELCSYYQSCCTDYTAECKP
QVTRGDVFTMPEDEYTVYDDGEEKNNATVHEQVGGPSLTSDLQAQSKGNPEQTPVLKPEE
EAPAPEVGASKPEGIDSRPETLHPGRPQPPAEEELCSGKPFDAFTDLKNGSLFAFRGQYC
YELDEKAVRPGYPKLIRDVWGIEGPIDAAFTRINCQGKTYLFKGSQYWRFEDGVLDPDYP
RNISDGFDGIPDNVDAALALPAHSYSGRERVYFFKGKQYWEYQFQHQPSQEECEGSSLSA
VFEHFAMMQRDSWEDIFELLFWGRTSAGTRQPQFISRDWHGVPGQVDAAMAGRIYISGMA
PRPSLAKKQRFRHRNRKGYRSQRGHSRGRNQNSRRPSRATWLSLESSEESNLGANNYDDY
RMDWLVPATCEPIQSVFFFSGDKYYRVNLRTRRVDTVDPPYPRSIAQYWLGCPAPGHL
>tr|Q3KR94|Q3KR94_RAT Vitronectin OS = Rattus norvegicus
OX = 10116 GN = Vtn PE = 1 SV = 1
SEQ ID NO: 2
MASLRPFFILALLALVSLADQESCKGRCTQGFMASKKCQCDELCTYYQSCCVDYMEQCKP
QVTRGDVFTMPEDEYWSYDYPEETKNSTSTGVQSENTSLHFNLKPRAEETIKPTTPDPQE
QSNTQEPEVGQQGVAPRPDTTDEGTSEFPEEELCSGKPFDAFTDLKNGSLFAFRGEYCYE
LDETAVRPGYPKLIQDVWGIEGPIDAAFTRINCQGKTYLFKGSQYWRFEDGVLDPDYPRN
ISEGFSGIPDNVDAALALPAHSYSGRERVYFFKGKQYWEYEFQQQPSQEECEGSSLSAVF
EHFALLQRDSWENIFELLFWGRSSDGAKGPQFISRDWHGVPGKVDAAMAGRIYITGSTFR
SVQAKKQKSGRRSRKRYRSRRGRGHSRSRSRSMSSRRPSRSVWFSLLSSEESGLGTYNYD
YDMNWRIPATCEPIQSVYFFSGDKYYRVNLRTRRVDSVNPPYPRSIAQYWLGCPTSEK
>sp|P29788|VTNC_MOUSE Vitronectin OS = Mus musculus OX = 10090
GN = Vtn PE = 1 SV = 2
SEQ ID NO: 3
MAPLRPFFILALVAWVSLADQESCKGRCTQGEMASKKCQCDELCTYYQSCCADYMEQCKP
QVTRGDVFTMPEDDYWSYDYVEEPKNNTNTGVQPENTSPPGDLNPRTDGTLKPTAFLDPE
EQPSTPAPKVEQQEEILRPDTTDQGTPEFPEEELCSGKPFDAFTDLKNGSLFAFRGQYCY
ELDETAVRPGYPKLIQDVWGIEGPIDAAFTRINCQGKTYLFKGSQYWRFEDGVLDPGYPR
NISEGFSGIPDNVDAAFALPAHRYSGRERVYFFKGKQYWEYEFQQQPSQEECEGSSLSAV
FEHFALLQRDSWENIFELLFWGRSSDGAREPQFISRNWHGVPGKVDAAMAGRIYVTGSLS
HSAQAKKQKSKRRSRKRYRSRRGRGHRRSQSSNSRRSSRSIWFSLFSSEESGLGTYNNYD
YDMDWLVPATCEPIQSVYFFSGDKYYRVNLRTRRVDSVNPPYPRSIAQYWLGCPTSEK

The term “biomarker” as used herein refers to a biological molecule present in an individual or on the surface of a call at varying concentrations useful for determining a phenotype of the cell. A biomarker may include but is not limited to, nucleic acids, proteins and variants and fragments thereof. A biomarker may be DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest.

Choline Acetyl Transferase (CHAT) refers to an enzyme that catalyzes the transfer of an acetyl group from the coenzyme acetyl-CoA to choline, yielding acetylcholine (ACh). In some embodiments, CHAT has at least about 70%, 80%, 85%, 90%, 91%, 92%, 93%, 94%, 95%, 96%, 97%, 98%, 99% or 100% sequence identity with SEQ ID NO: 4, SEQ ID NO: 5, SEQ ID NO: 6, or a fragment thereof.

>sp|P28329|CLAT_HUMAN Choline O-acetyltransferase OS = Homo
sapiens OX = 9606 GN = CHAT PE = 1 SV = 4
SEQ ID NO: 4
MGLRTAKKRGLGGGGKWKREEGGGTRGRREVRPACFLQSGGRGDPGDVGGPAGNPGCSPH
PRAATRPPPLPAHTPAHTPEWCGAASAEAAEPRRAGPHLCIPAPGLTKTPILEKVPRKMA
AKTPSSEESGLPKLPVPPLQQTLATYLQCMRHLVSEEQFRKSQAIVQQFGAPGGLGETLQ
QKLLERQEKTANWVSEYWLNDMYLNNRLALPVNSSPAVIFARQHFPGTDDQLRFAASLIS
GVLSYKALLDSHSIPTDCAKGQLSGQPLCMKQYYGLFSSYRLPGHTQDTLVAQNSSIMPE
PEHVIVACCNQFFVLDVVINFRRLSEGDLFTQLRKIVKMASNEDERLPPIGLLTSDGRSE
WAEARTVLVKDSTNRDSLDMIERCICLVCLDAPGGVELSDTHRALQLLHGGGYSKNGANR
WYDKSLQFVVGRDGTCGVVCEHSPFDGIVLVQCTEHLLKHVTQSSRKLIRADSVSELPAP
RRLRWKCSPEIQGHLASSAEKLQRIVKNLDFIVYKFDNYGKTFIKKQKCSPDAFIQVALQ
LAFYRLHRRLVPTYESASIRRFQEGRVDNIRSATPEALAFVRAVTDHKAAVPASEKLLLL
KDAIRAQTAYTVMAITGMAIDNHLLALRELARAMCKELPEMFMDETYLMSNRFVLSTSQV
PTTTEMFCCYGPVVPNGYGACYNPQPETILFCISSFHSCKETSSSKFAKAVEESLIDMRD
LCSLLPPTESKPLATKEKATRPSQGHQP
>sp|P32738|CLAT_RAT Choline O-acetyltransferase OS = Rattus
norvegicus OX = 10116 GN = Chat PE = 1 SV = 2
SEQ ID NO: 5
MPILEKAPQKMPVKASSWEELDLPKLPVPPLQQTLATYLQCMQHLVPEEQFRKSQAIVKR
FGAPGGLGETLQEKLLERQEKTANWVSEYWLNDMYLNNRLALPVNSSPAVIFARQHFQDT
NDQLRFAACLISGVLSYKTLLDSHSLPTDWAKGQLSGQPLCMKQYYRLFSSYRLPGHTQD
TLVAQKSSIMPEPEHVIVACCNQFFVLDVVINFRRLSEGDLFTQLRKIVKMASNEDERLP
PIGLLTSDGRSEWAKARTVLLKDSTNRDSLDMIERCICLVCLDGPGTGELSDTHRALQLL
HGGGCSLNGANRWYDKSLQFVVGRDGTCGVVCEHSPFDGIVLVQCTEHLLKHMMTSNKKL
VRADSVSELPAPRRLRLKCSPETQGHLASSAEKLQRIVKNLDFIVYKFDNYGKTFIKKQK
YSPDGFIQVALQLAYYRLYQRLVPTYESASIRRFQEGRVDNIRSATPEALAFVQAMTDHK
AAMPASEKLQLLQTAMQAHKQYTVMAITGMAIDNHLLALRELARDLCKEPPEMFMDETYL
MSNRFVLSTSQVPTTMEMFCCYGPVVPNGNGACYNPQPEAITFCISSFHSCKETSSVEFA
EAVGASLVDMRDLCSSRQPADSKPPAPKEKARGPSQAKQS
>sp|Q03059|CLAT_MOUSE Choline O-acetyltransferase OS = Mus
musculus OX = 10090 GN = Chat PE = 2 SV = 2
SEQ ID NO: 6
MPILEKVPPKMPVQASSCEEVLDLPKLPVPPLQQTLATYLQCMQHLVPEEQFRKSQAIVK
RFGAPGGLGETLQEKLLERQEKTANWVSEYWLNDMYLNNRLALPVNSSPAVIFARQHFQD
TNDQLRFAASLISGVLSYKALLDSQSIPTDWAKGQLSGQPLCMKQYYRLFSSYRLPGHTQ
DTLVAQKSSIMPEPEHVIVACCNQFFVLDVVINFRRLSEGDLFTQLRKIVKMASNEDERL
PPIGLLTSDGRSEWAKARTVLLKDSTNRDSLDMIERCICLVCLDGPGTGDLSDTHRALQL
LHGGGCSLNGANRWYDKSLQFVVGRDGTCGVVCEHSPFDGIVLVQCTEHLLKHMMTGNKK
LVRVDSVSELPAPRRLRWKCSPETQGHLASSAEKLQRIVKNLDFIVYKFDNYGKTFIKKQ
KCSPDGFIQVALQLAYYRLYQRLVPTYESASIRRFQEGRVDNIRSATPEALAFVQAMTDH
KAAVLASEKLQLLQRAIQAQTEYTVMAITGMAIDNHLLALRELARDLCKEPPEMFMDETY
LMSNRFILSTSQVPTTMEMFCCYGPVVPNGYGACYNPHAEAITFCISSFHGCKETSSVEF
AEAVGASLVDMRDLCSSRQPADSKPPTAKERARGPSQAKQS

“Serotonin receptors” or “5-hydroxytryptamine (5-HT) receptors” are G protein-coupled receptor and ligand-gated ion channels found in the central and peripheral nervous systems. Serotonin activates the serotonin receptors, mediating both excitatory and inhibitory neurotransmission. In some embodiments, serotonin receptors have at least about 70%, 80%, 85%, 90%, 91%, 92%, 93%, 94%, 95%, 96%, 97%, 98%, 99% or 100% sequence identity with SEQ ID NO: 7, SEQ ID NO: 8, SEQ ID NO: 9, or a fragment thereof.

>sp|P08908|5HT1A_HUMAN 5-hydroxytryptamine receptor 1A OS = Homo
sapiens OX = 9606 GN = HTR1A PE = 1 SV = 3
SEQ ID NO: 7
MDVLSPGQGNNTTSPPAPFETGGNTTGISDVTVSYQVITSLLLGTLIFCAVLGNACVVAA
IALERSLQNVANYLIGSLAVTDLMVSVLVLPMAALYQVLNKWTLGQVTCDLFIALDVLCC
TSSILHLCAIALDRYWAITDPIDYVNKRTPRRAAALISLTWLIGFLISIPPMLGWRTPED
RSDPDACTISKDHGYTIYSTFGAFYIPLLLMLVLYGRIFRAARFRIRKTVKKVEKTGADT
RHGASPAPQPKKSVNGESGSRNWRLGVESKAGGALCANGAVRQGDDGAALEVIEVHRVGN
SKEHLPLPSEAGPTPCAPASFERKNERNAEAKRKMALARERKTVKTLGIIMGTFILCWLP
FFIVALVLPFCESSCHMPTLLGAIINWLGYSNSLLNPVIYAYFNKDFQNAFKKIIKCKFC
RQ
>sp|P19327|5HT1A_RAT 5-hydroxytryptamine receptor 1A OS = Rattus
norvegicus OX = 10116 GN = Htr1a PE = 1 SV = 1
SEQ ID NO: 8
MDVFSFGQGNNTTASQEPFGTGGNVTSISDVTFSYQVITSLLLGTLIFCAVIGNACVVAA
IALERSLQNVANYLIGSLAVTDLMVSVLVLPMAALYQVLNKWTLGQVTCDLFIALDVLCC
TSSILHLCAIALDRYWAITDPIDYVNKRTPRRAAALISLTWLIGFLISIPPMLGWRTPED
RSDPDACTISKDHGYTIYSTFGAFYIPLLLMLVLYGRIFRAARFRIRKTVRKVEKKGAGT
SLGTSSAPPPKKSLNGQPGSGDWRRCAENRAVGTPCTNGAVRQGDDEATLEVIEVHRVGN
SKEHLPLPSESGSNSYAPACLERKNERNAEAKRKMALARERKTVKTLGIIMGTFILCWLP
FFIVALVLPFCESSCHMPALLGAIINWLGYSNSLLNPVIYAYFNKDFQNAFKKIIKCKFC
RR
>sp|Q64264|5HT1A_MOUSE 5-hydroxytryptamine receptor 1A OS = Mus
musculus OX = 10090 GN = Htr1a PE = 2 SV = 2
SEQ ID NO: 9
MDMFSLGQGNNTTTSLEPFGTGGNDTGLSNVTFSYQVITSLLLGTLIFCAVIGNACVVAA
IALERSLQNVANYLIGSLAVTDLMVSVLVLPMAALYQVLNKWTLGQVTCDLFIALDVLCC
TSSILHLCAIALDRYWAITDPIDYVNKRTPRRAAALISLTWLIGFLISIPPMLGWRTPED
RSNPNECTISKDHGYTIYSTFGAFYIPLLLMLVLYGRIFRAARFRIRKTVKKVEKKGAGT
SFGTSSAPPPKKSLNGQPGSGDCRRSAENRAVGTPCANGAVRQGEDDATLEVIEVHRVGN
SKGHLPLPSESGATSYVPACLERKNERTAEAKRKMALARERKTVKTLGIIMGTFILCWLP
FFIVALVLPFCESSCHMPELLGAIINWLGYSNSLLNPVIYAYFNKDFQNAFKKIIKCKFC
R

Gamma-Aminobutyric acid (GABA) acts as a trophic factor to modulate several essential developmental processes including neuronal proliferation, migration, and differentiation.

Neuronal nitric oxide synthase (nNOS) produces nitric oxide (NO) in the central and peripheral nervous systems. In some embodiments, nNOS has at least about 70%, 80%, 85%, 90%, 91%, 92%, 93%, 94%, 95%, 96%, 97%, 98%, 99% or 100% sequence identity with SEQ ID NO: 10, SEQ ID NO: 11, SEQ ID NO: 12, or a fragment thereof.

>sp|P29475|NOS1_HUMAN Nitric oxide synthase,
brain OS = Homo sapiens OX = 9606 GN = NOS1
PE = 1 SV = 2
SEQ ID NO: 10
MEDHMFGVQQIQPNVISVRLFKRKVGGLGFLVKERVSKPPVIISDLIRGG
AAEQSGLIQAGDIILAVNGRPLVDLSYDSALEVLRGIASETHVVLILRGP
EGFTTHLETTFTGDGTPKTIRVTQPLGPPTKAVDLSHQPPAGKEQPLAVD
GASGPGNGPQHAYDDGQEAGSLPHANGLAPRPPGQDPAKKATRVSLQGRG
ENNELLKEIEPVLSLLTSGSRGVKGGAPAKAEMKDMGIQVDRDLDGKSHK
PLPLGVENDRVFNDLWGKGNVPVVLNNPYSEKEQPPTSGKQSPTKNGSPS
KCPRFLKVKNWETEVVLTDTLHLKSTLETGCTEYICMGSIMHPSQHARRP
EDVRTKGQLFPLAKEFIDQYYSSIKRFGSKAHMERLEEVNKEIDTTSTYQ
LKDTELIYGAKHAWRNASRCVGRIQWSKLQVFDARDCTTAHGMFNYICNH
VKYATNKGNLRSAITIFPQRTDGKHDFRVWNSQLIRYAGYKQPDGSTLGD
PANVQFTEICIQQGWKPPRGRFDVLPLLLQANGNDPELFQIPPELVLEVP
IRHPKFEWFKDLGLKWYGLPAVSNMLLEIGGLEFSACPFSGWYMGTEIGV
RDYCDNSRYNILEEVAKKMNLDMRKTSSLWKDQALVEINIAVLYSFQSDK
VTIVDHHSATESFIKHMENEYRCRGGCPADWVWIVPPMSGSITPVFHQEM
LNYRLTPSFEYQPDPWNTHVWKGTNGTPTKRRAIGFKKLAEAVKFSAKLM
GQAMAKRVKATILYATETGKSQAYAKTLCEIFKHAFDAKVMSMEEYDIVH
LEHETLVLVVTSTFGNGDPPENGEKFGCALMEMRHPNSVQEERKSYKVRF
NSVSSYSDSQKSSGDGPDLRDNFESAGPLANVRFSVFGLGSRAYPHFCAF
GHAVDTLLEELGGERILKMREGDELCGQEEAFRTWAKKVFKAACDVFCVG
DDVNIEKANNSLISNDRSWKRNKFRLTFVAEAPELTQGLSNVHKKRVSAA
RLLSRQNLQSPKSSRSTIFVRLHTNGSQELQYQPGDHLGVFPGNHEDLVN
ALIERLEDAPPVNQMVKVELLEERNTALGVISNWTDELRLPPCTIFQAFK
YYLDITTPPTPLQLQQFASLATSEKEKQRLLVLSKGLQEYEEWKWGKNPT
IVEVLEEFPSIQMPATLLLTQLSLLQPRYYSISSSPDMYPDEVHLTVAIV
SYRTRDGEGPIHHGVCSSWLNRIQADELVPCFVRGAPSFHLPRNPQVPCI
LVGPGTGIAPFRSFWQQRQFDIQHKGMNPCPMVLVFGCRQSKIDHIYREE
TLQAKNKGVFRELYTAYSREPDKPKKYVQDILQEQLAESVYRALKEQGGH
IYVCGDVTMAADVLKAIQRIMTQQGKLSAEDAGVFISRMRDDNRYHEDIF
GVTLRTYEVTNRLRSESIAFIEESKKDTDEVFSS
>sp|P29476|NOS1_RAT Nitric oxide synthase,
brain OS = Rattus norvegicus OX = 10116
GN = Nos1 PE = 1 SV = 1
SEQ ID NO: 11
MEENTFGVQQIQPNVISVRLFKRKVGGLGFLVKERVSKPPVIISDLIRGG
AAEQSGLIQAGDIILAVNDRPLVDLSYDSALEVLRGIASETHVVLILRGP
EGFTTHLETTFTGDGTPKTIRVTQPLGPPTKAVDLSHQPSASKDQSLAVD
RVTGLGNGPQHAQGHGQGAGSVSQANGVAIDPTMKSTKANLQDIGEHDEL
LKEIEPVLSILNSGSKATNRGGPAKAEMKDTGIQVDRDLDGKSHKAPPLG
GDNDRVFNDLWGKDNVPVILNNPYSEKEQSPTSGKQSPTKNGSPSRCPRF
LKVKNWETDVVLTDTLHLKSTLETGCTEHICMGSIMLPSQHTRKPEDVRT
KDQLFPLAKEFLDQYYSSIKRFGSKAHMDRLEEVNKEIESTSTYQLKDTE
LIYGAKHAWRNASRCVGRIQWSKLQVFDARDCTTAHGMFNYICNHVKYAT
NKGNLRSAITIFPQRTDGKHDFRVWNSQLIRYAGYKQPDGSTLGDPANVQ
FTEICIQQGWKAPRGRFDVLPLLLQANGNDPELFQIPPELVLEVPIRHPK
FDWFKDLGLKWYGLPAVSNMLLEIGGLEFSACPFSGWYMGTEIGVRDYCD
NSRYNILEEVAKKMDLDMRKTSSLWKDQALVEINIAVLYSFQSDKVTIVD
HHSATESFIKHMENEYRCRGGCPADWVWIVPPMSGSITPVFHQEMLNYRL
TPSFEYQPDPWNTHVWKGTNGTPTKRRAIGFKKLAEAVKFSAKLMGQAMA
KRVKATILYATETGKSQAYAKTLCEIFKHAFDAKAMSMEEYDIVHLEHEA
LVLVVTSTFGNGDPPENGEKFGCALMEMRHPNSVQEERKSYKVRFNSVSS
YSDSRKSSGDGPDLRDNFESTGPLANVRFSVFGLGSRAYPHFCAFGHAVD
TLLEELGGERILKMREGDELCGQEEAFRTWAKKVFKAACDVFCVGDDVNI
EKPNNSLISNDRSWKRNKFRLTYVAEAPDLTQGLSNVHKKRVSAARLLSR
QNLQSPKFSRSTIFVRLHTNGNQELQYQPGDHLGVFPGNHEDLVNALIER
LEDAPPANHVVKVEMLEERNTALGVISNWKDESRLPPCTIFQAFKYYLDI
TTPPTPLQLQQFASLATNEKEKQRLLVLSKGLQEYEEWKWGKNPTMVEVL
EEFPSIQMPATLLLTQLSLLQPRYYSISSSPDMYPDEVHLTVAIVSYHTR
DGEGPVHHGVCSSWLNRIQADDVVPCFVRGAPSFHLPRNPQVPCILVGPG
TGIAPFRSFWQQRQFDIQHKGMNPCPMVLVFGCRQSKIDHIYREETLQAK
NKGVFRELYTAYSREPDRPKKYVQDVLQEQLAESVYRALKEQGGHIYVCG
DVTMAADVLKAIQRIMTQQGKLSEEDAGVFISRLRDDNRYHEDIFGVTLR
TYEVTNRLRSESIAFIEESKKDADEVFSS
>sp|Q9Z0J4|NOS1_MOUSE Nitric oxide synthase,
brain OS = Mus musculus OX = 10090 GN = Nos1
PE = 1 SV = 1
SEQ ID NO: 12
MEEHTFGVQQIQPNVISVRLFKRKVGGLGFLVKERVSKPPVIISDLIRGG
AAEQSGLIQAGDIILAVNDRPLVDLSYDSALEVLRGIASETHVVLILRGP
EGFTTHLETTFTGDGTPKTIRVTQPLGTPTKAVDLSRQPSASKDQPLAVD
RVPGPSNGPQHAQGRGQGAGSVSQANGVAIDPTMKNTKANLQDSGEQDEL
LKEIEPVLSILTGGGKAVNRGGPAKAEMKDTGIQVDRDLDGKLHKAPPLG
GENDRVFNDLWGKGNVPVVLNNPYSENEQSPASGKQSPTKNGSPSRCPRF
LKVKNWETDVVLTDTLHLKSTLETGCTEQICMGSIMLPSHHIRKSEDVRT
KDQLFPLAKEFLDQYYSSIKRFGSKAHMDRLEEVNKEIESTSTYQLKDTE
LIYGAKHAWRNASRCVGRIQWSKLQVFDARDCTTAHGMFNYICNHVKYAT
NKGNLRSAITIFPQRTDGKHDFRVWNSQLIRYAGYKQPDGSTLGDPANVE
FTEICIQQGWKPPRGRFDVLPLLLQANGNDPELFQIPPELVLEVPIRHPK
FDWFKDLGLKWYGLPAVSNMLLEIGGLEFSACPFSGWYMGTEIGVRDYCD
NSRYNILEEVAKKMDLDMRKTSSLWKDQALVEINIAVLYSFQSDKVTIVD
HHSATESFIKHMENEYRCRGGCPADWVWIVPPMSGSITPVFHQEMLNYRL
TPSFEYQPDPWNTHVWKGTNGTPTKRRAIGFKKLAEAVKFSAKLMGQAMA
KRVKATILYATETGKSQAYAKTLCEIFKHAFDAKAMSMEEYDIVHLEHEA
LVLVVTSTFGNGDPPENGEKFGCALMEMRHPNSVQEERKSYKVRFNSVSS
YSDSRKSSGDGPDLRDNFESTGPLANVRFSVFGLGSRAYPHFCAFGHAVD
TLLEELGGERILKMREGDELCGQEEAFRTWAKKVFKAACDVFCVGDDVNI
EKANNSLISNDRSWKRNKFRLTYVAEAPELTQGLSNVHKKRVSAARLLSR
QNLQSPKSSRSTIFVRLHTNGNQELQYQPGDHLGVFPGNHEDLVNALIER
LEDAPPANHVVKVEMLEERNTALGVISNWKDESRLPPCTIFQAFKYYLDI
TTPPTPLQLQQFASLATNEKEKQRLLVLSKGLQEYEEWKWGKNPTMVEVL
EEFPSIQMPATLLLTQLSLLQPRYYSISSSPDMYPDEVHLTVAIVSYHTR
DGEGPVHHGVCSSWLNRIQADDVVPCFVRGAPSFHLPRNPQVPCILVGPG
TGIAPFRSFWQQRQFDIQHKGMNPCPMVLVFGCRQSKIDHIYREETLQAK
NKGVFRELYTAYSREPDRPKKYVQDVLQEQLAESVYRALKEQGGHIYVCG
DVTMAADVLKAIQRIMTQQGKLSEEDAGVFISRLRDDNRYHEDIFGVTLR
TYEVTNRLRSESIAFIEESKKDTDEVFSS

Glial fibrillary acidic protein (GFAP) is a class-III intermediate filament. During the development of the central nervous system, GFAP is a cell-specific marker that distinguishes astrocytes from other glial cells. In some embodiments, GFAP has at least about 70%, 80%, 85%, 90%, 91%, 92%, 93%, 94%, 95%, 96%, 97%, 98%, 99% or 100% sequence identity with SEQ ID NO: 13, SEQ ID NO: 14, SEQ ID NO: 15, or a fragment thereof.

>sp|P14136|GFAP_HUMAN Glial fibrillary acidic
protein OS = Homo sapiens OX = 9606 GN = GFAP
PE = 1 SV = 1
SEQ ID NO: 13
MERRRITSAARRSYVSSGEMMVGGLAPGRRLGPGTRLSLARMPPPLPTRV
DFSLAGALNAGFKETRASERAEMMELNDRFASYIEKVRFLEQQNKALAAE
LNQLRAKEPTKLADVYQAELRELRLRLDQLTANSARLEVERDNLAQDLAT
VRQKLQDETNLRLEAENNLAAYRQEADEATLARLDLERKIESLEEEIRFL
RKIHEEEVRELQEQLARQQVHVELDVAKPDLTAALKEIRTQYEAMASSNM
HEAEEWYRSKFADLTDAAARNAELLRQAKHEANDYRRQLQSLTCDLESLR
GTNESLERQMREQEERHVREAASYQEALARLEEEGQSLKDEMARHLQEYQ
DLLNVKLALDIEIATYRKLLEGEENRITIPVQTFSNLQIRETSLDTKSVS
EGHLKRNIVVKTVEMRDGEVIKESKQEHKDVM
>sp|P47819|GFAP_RAT Glial fibrillary acidic
protein OS = Rattus norvegicus OX = 10116
GN = Gfap PE = 1 SV = 2
SEQ ID NO: 14
MERRRITSARRSYASSETMVRGHGPTRHLGTIPRLSLSRMTPPLPARVDF
SLAGALNAGFKETRASERAEMMELNDRFASYIEKVRFLEQQNKALAAELN
QLRAKEPTKLADVYQAELRELRLRLDQLTTNSARLEVERDNLTQDLGTLR
QKLQDETNLRLEAENNLAVYRQEADEATLARVDLERKVESLEEEIQFLRK
IHEEEVRELQEQLAQQQVHVEMDVAKPDLTAALREIRTQYEAVATSNMQE
TEEWYRSKFADLTDVASRNAELLRQAKHEANDYRRQLQALTCDLESLRGT
NESLERQMREQEERHARESASYQEALARLEEEGQSLKEEMARHLQEYQDL
LNVKLALDIEIATYRKLLEGEENRITIPVQTFSNLQIRETSLDTKSVSEG
HLKRNIVVKTVEMRDGEVIKESKQEHKDVM
>sp|P03995|GFAP_MOUSE Glial fibrillary acidic
protein OS = Mus musculus OX = 10090 GN = Gfap
PE = 1 SV = 4
SEQ ID NO: 15
MERRRITSARRSYASETVVRGLGPSRQLGTMPRFSLSRMTPPLPARVDES
LAGALNAGFKETRASERAEMMELNDREASYIEKVRFLEQQNKALAAELNQ
LRAKEPTKLADVYQAELRELRLRLDQLTANSARLEVERDNFAQDLGTLRQ
KLQDETNLRLEAENNLAAYRQEADEATLARVDLERKVESLEEEIQFLRKI
YEEEVRELREQLAQQQVHVEMDVAKPDLTAALREIRTQYEAVATSNMQET
EEWYRSKFADLTDAASRNAELLRQAKHEANDYRRQLQALTCDLESLRGTN
ESLERQMREQEERHARESASYQEALARLEEEGQSLKEEMARHLQEYQDLL
NVKLALDIEIATYRKLLEGEENRITIPVQTFSNLQIRETSLDTKSVSEGH
LKRNIVVKTVEMRDGEVIKDSKQEHKDVVM

Enteric neural crest cells express SOX10, which directs the activity of other genes that signal neural crest cells to become more specific cell types including enteric nerves. In some embodiments, SOX10 has at least about 70%, 80%, 85%, 90%, 91%, 92%, 93%, 94%, 95%, 96%, 97%, 98%, 99% or 100% sequence identity with SEQ ID NO: 16, SEQ ID NO: 17, SEQ ID NO: 18, or a fragment thereof.

>sp|P56693|SOX10_HUMAN Transcription factor
SOX-10 OS = Homo sapiens OX = 9606 GN = SOX10
PE = 1 SV = 1
SEQ ID NO: 16
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGEAEQGGT
AAIQAHYKSAHLDHRHPGEGSPMSDGNPEHPSGQSHGPPTPPTTPKTELQ
SGKADPKRDGRSMGEGGKPHIDEGNVDIGEISHEVMSNMETFDVAELDQY
LPPNGHPGHVSSYSAAGYGLGSALAVASGHSAWISKPPGVALPTVSPPGV
DAKAQVKTETAGPQGPPHYTDQPSTSQIAYTSLSLPHYGSAFPSISRPQF
DYSDHQPSGPYYGHSGQASGLYSAFSYMGPSQRPLYTAISDPSPSGPQSH
SPTHWEQPVYTTLSRP
>sp|O55170|SOX10_RAT Transcription factor
SOX-10 OS = Rattus norvegicus OX = 10116
GN = Sox10 PE = 1 SV = 1
SEQ ID NO: 17
MAEEQDLSEVELSPVGSEEPRCLSPSSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGETDQGGA
AAIQAHYKSAHLDHRHPEEGSPMSDGNPEHPSGQSHGPPTPPTTPKTELQ
SGKADPKRDGRSLGEGGKPHIDEGNVDIGEISHEVMSNMETFDVTELDQY
LPPNGHPGHVGSYSAAGYGLSSALAVASGHSAWISKPPGVALPTVSPPAV
DAKAQVKTETTGPQGPPHYTDQPSTSQIAYTSLSLPHYGSAFPSISRPQF
DYSDHQPSGPYYGHAGQASGLYSAFSYMGPSQRPLYTAISDPSPSGPQSH
SPTHWEQPVYTTLSRP
>sp|Q04888|SOX10_MOUSE Transcription factor
SOX-10 OS = Mus musculus OX = 10090 GN = Sox10
PE = 1 SV = 2
SEQ ID NO: 18
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGEAEQGGA
AAIQAHYKSAHLDHRHPEEGSPMSDGNPEHPSGQSHGPPTPPTTPKTELQ
SGKADPKRDGRSLGEGGKPHIDFGNVDIGEISHEVMSNMETFDVTELDQY
LPPNGHPGHVGSYSAAGYGLGSALAVASGHSAWISKPPGVALPTVSPPGV
DAKAQVKTETTGPQGPPHYTDQPSTSQIAYTSLSLPHYGSAFPSISRPQF
DYSDHQPSGPYYGHAGQASGLYSAFSYMGPSQRPLYTAISDPSPSGPQSH
SPTHWEQPVYTTLSRP

Enteric neural crest cells express CD24. In some embodiments, CD24 has about 70%, 80%, 85%, 90%, 91%, 92%, 93%, 94%, 95%, 96%, 97%, 98%, 99% or 100% sequence identity with SEQ ID NO: 19, or a fragment thereof.

SEQ ID NO: 19
MGRAMVARLGLGLLLLALLLPTQIYSSETTTGTSSNSSQSTSNSGLAPNP
TNATTKAAGGALQSTASLFVVSLSLLHLYS

Enteric neural crest cells express CD45RA. In some embodiments, CD45RA has at least about 70%, 80%, 85%, 90%, 91%, 92%, 93%, 94%, 95%, 96%, 97%, 98%, 99% or 100% sequence identity with GENBANK (R) Accession Nos. NP_002829.3, NP_563578.2, NP_563578.2, and NP_002829.3, all of which are incorporated herein by reference.

Enteric neural crest cells express CD57. In some embodiments, CD57 has at least about 70%, 80%, 85%, 90%, 91%, 92%, 93%, 94%, 95%, 96%, 97%, 98%, 99% or 100% sequence identity with SEQ ID NO: 20, SEQ ID NO: 21, SEQ ID NO: 22, SEQ ID NO: 23, or a fragment thereof.

SEQ ID NO: 20
MGNEEPWVQPALEMPKRRDILAIVLIVLPWTLLITVWHQSTLAPLLAVHK
DEGSDPRRETPPGADPREYCTSDRDIVEVVRTEYVYTRPPPWSDTLPTIH
VVTPTYSRPVQKAELTRMANTLLHVPNLHWLVVEDAPRRTPLTARLLRDT
GLNYTHLHVETPRNYKLRGDARDPRIPRGTMQRNLALRWLRETFPRNSSQ
PGVVYFADDDNTYSLELFEEMRSTRRVSVWPVAFVGGLRYEAPRVNGAGK
VVGWKTVFDPHRPFAIDMAGFAVNLRLILQRSQAYFKLRGVKGGYQESSL
LRELVTLNDLEPKAANCTKILVWHTRTEKPVLVNEGKKGFTDPSVEI
SEQ ID NO: 21
MGNEEPWVQP ALEMPKRRDI LAIVLIVLPW TLLITVWHQS
TLAPLLAVHK DEGSDPRRET PPGADPREYC TSDRDIVEVV
RTEYVYTRPP PWSDTLPTIH VVTPTYSRPV QKAELTRMAN
TLLHVPNLHW LVVEDAPRRT PLTARLLRDT GLNYTHLHVE
TPRNYKLRGD ARDPRIPRGTMQRNLALRWL RETFPRNSSQ
PGVVYFADDD NTYSLELFEE MRSTRRVSVW PVAFVGGLRY
EAPRVNGAGKVVGWKTVFDPHRPFAIDMAGFAVNLRLILQRSQ
AYFKLRGVKGGYQESSLLRELVT LNDL EPKAANCTKI
LVWHTRTEKP VLVNEGKKGF TDPSVEI
SEQ ID NO: 22
MPKRRDILAI VLIVLPWTLL ITVWHQSTLA PLLAVHKDEG
SDPRRETPPG ADPREYCTSDRDIVEVVRTE YVYTRPPPWS
DTLPTIHVVT PTYSRPVQKA ELTRMANTLL HVPNLH
WLVVEDAPRRTPLT ARLLRDTGLN YTHLHVETPR
NYKLRGDARD PRIPRGTMQR NLALRWLRETFPRNSSQPGV
VYFADDDNTY SLELFEEMRS TRRVSVWPVA FVGGLRYEAP
RVNGAGKVVGWKTVFDPHRP FAIDMAGFAV NLRLILQRSQ
AYFKLRGVKG GYQESSLLRE LVTLNDLEPKAANCTKILVW
HTRTEKPVLV NEGKKGFTDP SVEI
SEQ ID NO: 23
MPKRRDILAI VLIVLPWTLL ITVWHQSTLA PLLAVHKDEG
SDPRRETPPG ADPREYCTSDRDIVEVVRTE YVYTRPPPWS
DTLPTIHVVT PTYSRPVQKA ELTRMANTLL HVPNLHWLVV
EDAPRRTPLT ARLLRDTGLN YTHLHVETPR NYKLRGDARD
PRIPRGTMQR NLALRWLRETFPRNSSQPGV VYFADDDNTY
SLELFEEMRS TRRVSVWPVA FVGGLRYEAP RVNGAGKVVG
WKTVFDPHRP FAIDMAGFAV NLRLILQRSQ AYFKLRGVKG
GYQESSLLRE LVTLNDLEPKAANCTKILVW HTRTEKPVLV
NEGKKGFTDP SVEI

Enteric neural crest cells express CD63. In some embodiments, CD63 has at least about 70%, 80%, 85%, 90%, 91%, 92%, 93%, 94%, 95%, 96%, 97%, 98%, 99% or 100% sequence identity with SEQ ID NO: 24, or a fragment thereof.

SEQ ID NO: 24
MAVEGGMKCV KFLLYVLLLA FCACAVGLIA VGVGAQLVLS
QTIIQGATPG SLLPVVIIAVGVFLFLVAFV GCCGACKENY
CLMITFAIFL SLIMLVEVAA AIAGYVFRDK VMSEFNNNFRQQ
MENYPKNN HTASILDRMQ ADFKCCGAAN YTDWEKIPSM
SKNRVPDSCC INVTVGCGINFNEKAIHKEG CVEKIGGWLR
KNVLVVAAAA LGIAFVEVLG IVFACCLVKS IRSGYEVM

Enteric neural crest cells express CD71. In some embodiments, CD71 has at least about 70%, 80%, 85%, 90%, 91%, 92%, 93%, 94%, 95%, 96%, 97%, 98%, 99% or 100% sequence identity with SEQ ID NO: 25, or a fragment thereof.

SEQ ID NO. 25
MMDQARSAFS NLFGGEPLSY TRFSLARQVD GDNSHVEMKL
AVDEEENADN NTKANVTKPK RCSGSICYGT IAVIVFFLIG
FMIGYLGYCK GVEPKTECER LAGTESPVRE EPGEDFPAAR
RLYWDDLKRK LSEKLDSTDF TGTIKLLNEN SYVPREAGSQ
KDENLALYVE NQFREFKLSK VWRDQHFVKI QVKDSAQNSV
IIVDKNGRLV YLVENPGGYV AYSKAATVTG KLVHANFGTK
KDFEDLYTPV NGSIVIVRAG KITFAEKVAN AESLNAIGVL
IYMDQTKFPI VNAELSFFGH AHLGTGDPYT PGFPSFNHTQ
FPPSRSSGLP NIPVQTISRA AAEKLFGNME GDCPSDWKTD
STCRMVTSES KNVKLTVSNV LKEIKILNIF GVIKGFVEPD
HYVVVGAQRD AWGPGAAKSG VGTALLLKLA QMFSDMVLKD
GFQPSRSIIF ASWSAGDFGS VGATEWLEGY LSSLHLKAFT
YINLDKAVLG TSNFKVSASP LLYTLIEKTM QNVKHPVTGQ
FLYQDSNWAS KVEKLTLDNA AFPFLAYSGI PAVSFCFCED
TDYPYLGTTM DTYKELIERI PELNKVARAA AEVAGQFVIK
LTHDVELNLD YERYNSQLLS FVRDLNQYRA DIKEMGLSLQ
WLYSARGDFF RATSRLTTDF GNAEKTDRFV MKKLNDRVMR
EGPQMMLLLT DARPSNHFLS PLLSLHRg

Enteric neural crest cells express CD121b. In some embodiments, CD121b has at least about 70%, 80%, 85%, 90%, 91%, 92%, 93%, 94%, 95%, 96%, 97%, 98%, 99% or 100% sequence identity with SEQ ID NO: 26, or a fragment thereof.

SEQ ID NO. 26
MWAQDGALWL LPALQEDSGT YVCTTRNASY CDKMSIELRV
FENTDAFLPF ISYPQILTLS TSGVLVCPDL SEFTRDKTDV
KIQWYKDSLL LDKDNEKFLS VRGTTHLLVH DVALEDAGYY
RCVLTFAHEG QQYNITRSIE LRIKKKKEET IPVIISPLKT
ISASLGSRLT IPCKVFLGTG TPLTTMLWWT ANDTHIESAY
PGGRVTEGPR QEYSENNENY IEVPLIFDPV TREDLHMDFK
CVVHNTLSFQ TLRTTVKEAS STFSWGIVLA PLSLAFLVLG
GIWMHRRCKH RTGKADGLTV LWPHHQDFQS YPK

Enteric neural crest cells express CD147. In some embodiments, CD147 has at least about 70%, 80%, 85%, 90%, 91%, 92%, 93%, 94%, 95%, 96%, 97%, 98%, 99% or 100% sequence identity with SEQ ID NO: 27, or a fragment thereof.

SEQ ID NO. 27
MAAALFVLLG FALLGTHGAS GAAGFVQAPL SQQRWVGGSV
ELHCEAVGSP VPEIQWWFEG QGPNDTCSQL WDGARLDRVH
IHATYHQHAA STISIDTLVE EDTGTYECRA SNDPDRNHLT
RAPRVKWVRA QAVVLVLEPG TVFTTVEDLG SKILLTCSLN
DSATEVTGHR WLKGGVVLKE DALPGQKTEF KVDSDDQWGE
YSCVFLPEPM GTANIQLHGP PRVKAVKSSE HINEGETAML
VCKSESVPPV TDWAWYKITD SEDKALMNGS ESRFFVSSSQ
GRSELHIENL NMEADPGQYR CNGTSSKGSD QAIITLRVRS
HLAALWPFLG IVAEVLVLVT IIFIYEKRRK PEDVLDDDDA
GSAPLKSSGQ HQNDKGKNVR QRNSS

Enteric neural crest cells express CD148. In some embodiments, CD148 has at least about 70%, 80%, 85%, 90%, 91%, 92%, 93%, 94%, 95%, 96%, 97%, 98%, 99% or 100% sequence identity with SEQ ID NO: 28, or a fragment thereof.

SEQ ID NO: 28
MTRGGGSGSS RGSRDRVAAR WGWAPLAPPR EAPARSGTRP
PRGSRARLRR VAAAAAAAAM SPGKPGAGGA GTRRTGWRRR
RRRRRQEAAT TVPGLGRTAG PDSRVRGTFQ GARGMKPAAR
EARLPPRSPG LRWALPLLLL LLRLGQILCA GGTPSPIPDP
SVATVATGEN GITQISSTAE SFHKQNGTGT PQVETNTSED
GESSGANDSL RTPEQGSNGT DGASQKTPSS TEPIPVSDLR
VALTGVRKAA LSWSNGNGTA SCRVLLESIG SHEELTQDSR
LQVNISGLKP GVQYNINPYL LQSNKTKGDP LGTEGGLDAS
NTERSRAGSP TAPVHDESLV GPVDPSSGQQ SRDTEVLLVG
LEPGTRYNAT VYSQAANGTE GQPQAIEFRT NAIQVEDVTA
VNISATSLTL IWKVSDNESS SNYTYKIHVA GETDSSNLNV
SEPRAVIPGL RSSTFYNITV CPVLGDIEGT PGFLQVHTPP
VPVSDFRVTV VSTTEIGLAW SSHDAESFQM HITQEGAGNS
RVEITTNQSI IIGGLFPGTK YCFEIVPKGP NGTEGASRTV
CNRTVPSAVF DIHVVYVTTT EMWLDWKSPD GASEYVYHLV
IESKHGSNHT STYDKAITLQ GLIPGTLYNI TISPEVDHVW
GDPNSTAQYT RPSNVSNIDV STNTTAATLS WQNFDDASPT
YSYCLLIEKA GNSSNATQVV TDIGITDATV TELIPGSSYT
VEIFAQVGDG IKSLEPGRKS FCTDPASMAS FDCEVVPKEP
ALVLKWTCPP GANAGFELEV SSGAWNNATH LESCSSENGT
EYRTEVTYLN FSTSYNISIT TVSCGKMAAP TRNTCTTGIT
DPPPPDGSPN ITSVSHNSVK VKFSGFEASH GPIKAYAVIL
TTGEAGHPSA DVLKYTYEDE KKGASDTYVT YLIRTEEKGR
SQSLSEVLKY EIDVGNESTT LGYYNGKLEP LGSYRACVAG
FTNITFHPQN KGLIDGAESY VSFSRYSDAV SLPQDPGVIC
GAVFGCIFGA LVIVTVGGFI FWRKKRKDAK NNEVSFSQIK
PKKSKLIRVE NFEAYFKKQQ ADSNCGFAEE YEDLKLVGIS
QPKYAAELAE NRGKNRYNNV LPYDISRVKL SVQTHSTDDY
INANYMPGYH SKKDFIATQG PLPNTLKDFW RMVWEKNVYA
IIMLTKCVEQ GRTKCEEYWP SKQAQDYGDI TVAMTSEIVL
PEWTIRDFTV KNIQTSESHP LRQFHFTSWP DHGVPDTTDL
LINFRYLVRD YMKQSPPESP ILVHCSAGVG RIGTFIAIDR
LIYQIENENT VDVYGIVYDL RMHRPLMVQT EDQYVFLNQC
VLDIVRSQKD SKVDLIYQNT TAMTIYENLA PVTTFGKING
YIA

In some embodiments, CD193 has at least about 70%, 80%, 85%, 90%, 91%, 92%, 93%, 94%, 95%, 96%, 97%, 98%, 99% or 100% sequence identity with Genbank accession number XP_011531637.1, XP_006713023.1, NP_001158152.1, NP_847898.1, NP_847899.1, AAI30321.1, AAI10298.1, XP_016861175.1, XP_016861174.1, NP_001828.1, AAI30319.1, or ACN11153.1.

Enteric neural crest cells express CD193. In some embodiments, CD193 has at least about 70%, 80%, 85%, 90%, 91%, 92%, 93%, 94%, 95%, 96%, 97%, 98%, 99% or 100% sequence identity with SEQ ID NO: 29, or a fragment thereof.

SEQ ID NO: 29
MLLIIVIIVIV NYCDCTCVT DKMCIFFTAA VDWIMPFGIR
MLLRAHKPGS SRRSEMTTSL DTVETFGTTS YYDDVGLLCE
KADTRALMAQ FVPPLYSLVF TVGLLGNVVV VMILIKYRRL
RIMTNIYLLN LAISDLLFLV TLPFWIHYVR GHNWVFGHGM
CKLLSGFYHT GLYSEIFFII LLTIDRYLAI VHAVFALRAR
TVTFGVITSI VTWGLAVLAA LPEFIFYETE ELFEETLCSA
LYPEDTVYSW RHFHTLRMTI FCLVLPLLVM AICYTGIIKT
LLRCPSKKKY KAIRLIFVIM AVFFIFWTPY NVAILLSSYQ
SILFGNDCER SKHLDLVMLV TEVIAYSHCC MNPVIYAFVG
ERFRKYLRHF FHRHLLMHLG RYIPFLPSEK LERTSSVSPS
TAEPELSIVF

In some embodiments, CD193 has at least about 70%, 80%, 85%, 90%, 91%, 92%, 93%, 94%, 95%, 96%, 97%, 98%, 99% or 100% sequence identity with Genbank accession number XP_011531637.1, XP_006713023.1, NP_001158152.1, NP_847898.1, NP_847899.1, AAI30321.1, AAI10298.1, XP_016861175.1, XP_016861174.1, NP_001828.1, AAI30319.1, or ACN11153.1.

Enteric neural crest cells express CD243. In some embodiments, CD243 has at least about 70%, 80%, 85%, 90%, 91%, 92%, 93%, 94%, 95%, 96%, 97%, 98%, 99% or 100% sequence identity with SEQ ID NO: 30 or a fragment thereof.

SEQ ID NO: 30
MSVNLQGDQR GATEARTFLL EIQPVSQFLE ESAFSQSGPG
AVICGLSTKV GVSSSKISRL GGRSKEREVG MDLEGDRNGG
AKKKNFFKLN NKSEKDKKEK KPTVSVFSMF RYSNWLDKLY
MVVGTLAAII HGAGLPLMML VFGEMTDIFA NAGNLEDLMS
NITNRSDIND TGFFMNLEED MTRYAYYYSG IGAGVLVAAY
IQVSFWCLAA GRQIHKIRKQ FFHAIMRQEI GWFDVHDVGE
LNTRLTDDVS KINEGIGDKI GMFFQSMATF FTGFIVGFTR
GWKLTLVILA ISPVLGLSAA VWAKILSSFT DKELLAYAKA
GAVAEEVLAA IRTVIAFGGQ KKELERYNKN LEEAKRIGIK
KAITANISIG AAFLLIYASY ALAFWYGTTL VLSGEYSIGQ
VLTVFFSVLI GAFSVGQASP SIEAFANARG AAYEIFKIID
NKPSIDSYSK SGHKPDNIKG NLEFRNVHFS YPSRKEVKIL
KGLNLKVQSG QTVALVGNSG CGKSTTVQLM QRLYDPTEGM
VSVDGQDIRT INVRFLREII GVVSQEPVLF ATTIAENIRY
GRENVTMDEI EKAVKEANAY DFIMKLPHKF DTLVGERGAQ
LSGGQKQRIA IARALVRNPK ILLLDEATSA LDTESEAVVQ
VALDKARKGR TTIVIAHRLS TVRNADVIAG FDDGVIVEKG
NHDELMKEKG IYFKLVTMQT AGNEVELENA ADESKSEIDA
LEMSSNDSRS SLIRKRSTRR SVRGSQAQDR KLSTKEALDE
SIPPVSFWRI MKLNLTEWPY FVVGVFCAII NGGLQPAFAI
IFSKIIGVFT RIDDPETKRQ NSNLFSLLFL ALGIISFITF
FLQGFTFGKA GEILTKRLRY MVERSMLRQD VSWFDDPKNT
TGALTTRLAN DAAQVKGAIG SRLAVITQNI ANLGTGIIIS
FIYGWQLTLL LLAIVPIIAI AGVVEMKMLS GQALKDKKEL
EGSGKIATEA IENFRTVVSL TQEQKFEHMY AQSLQVPYRN
SLRKAHIFGI TESFTQAMMY FSYAGCFRFG AYLVAHKLMS
FEDVLLVFSA VVFGAMAVGQ VSSFAPDYAK AKISAAHIIM
IIEKTPLIDS YSTEGLMPNT LEGNVTFGEV VENYPTRPDI
PVLQGLSLEV KKGQTLALVG SSGCGKSTVV QLLERFYDPL
AGKVLLDGKE IKRLNVQWLR AHLGIVSQEP ILFDCSIAEN
IAYGDNSRVV SQEEIVRAAK EANIHAFIES LPNKYSTKVG
DKGTQLSGGQ KQRIAIARAL VRQPHILLLD EATSALDTES
EKVVQEALDK AREGRTCIVI AHRLSTIQNA
DLIVVFQNGR VKEHGTHQQL LAQKGIYFSM VSVQAGTKRQ

In some embodiments, CD243 has at least about 70%, 80%, 85%, 90%, 91%, 92%, 93%, 94%, 95%, 96%, 97%, 98%, 99% or 100% sequence identity with Genbank accession number NP_001335875.1, NP 001335874.1, NP_001335873.1, NP_000918.2, AAI30425.1, or KIH63939.1.

Enteric neural crest cells express CD275. In some embodiments, CD275 has at least about 70%, 80%, 85%, 90%, 91%, 92%, 93%, 94%, 95%, 96%, 97%, 98%, 99% or 100% sequence identity with SEQ ID NO: 31, or a fragment thereof.

SEQ ID NO: 31
MVGSDVELSC ACPEGSRFDL MDVYVYWQTS ESKTVVTYHI
PQNSSLENVD SRYRNRALMS PAGMLRGDFS LRLFNVTPQD
EQKFHCLVLS QSLGFQEVLS VEVTLHVAAN FSVPVVSAPH
SPSQDELTFT CTSINGYPRP NVYWINKTDN SLLDQALQND
TVELNMRGLY DVVSVLRIAR TPSVNIGCCI ENVLLQQNLT
VGSQTGNDIG ERDKITENPV STGEKNAATW SILAVLCLLV
VVAVAIGWVC RDRCLQHSYA GAWAVSPETE LTVSRHGFEQ
TTDVLPFILK SSLGASCEPT AFPLPPAAPG PCAHLFIWML
AECTPCSPVW SSIS

In some embodiments, CD275 has at least about 70%, 80%, 85%, 90%, 91%, 92%, 93%, 94%, 95%, 96%, 97%, 98%, 99% or 100% sequence identity with Genbank accession number XP_011527818.1, XP 011527816.1, NP_001382847.1, NP_001269981.1, NP 001269980.1, NP_001269979.1, NP_056074.1, XP_024307828.1, or NP_001352688.1.

Enteric glial cells express PMP22. In some embodiments, PMP22 has at least about 70%, 80%, 85%, 90%, 91%, 92%, 93%, 94%, 95%, 96%, 97%, 98%, 99% or 100% sequence identity with SEQ ID NO: 32, or a fragment thereof.

SEQ ID NO: 32
MLLLLLSIIV LHVAVLVLLF VSTIVSQWIV GNGHATDLWQ
NCSTSSSGNV HHCFSSSPNE WLQSVQATMI LSIIFSILSL
FLFFCQLFTL TKGGRFYITG IFQILAGLCV MSAAAIYTVR
HPEWHLNSDY SYGFAYILAW VAFPLALLSG VIYVILRKRE

In some embodiments, PMP22 has at least about 70%, 80%, 85%, 90%, 91%, 92%, 93%, 94%, 95%, 96%, 97%, 98%, 99% or 100% sequence identity with Genbank accession number CAG46751.1, CAG46729.1, NP_001268384.1, NP_001317072.1, NP_001268385.1, NP_696997.1, NP 696996.1, NP 000295.1, XP_024306574.1, or NP_061133.1

In some embodiments, the one or plurality of cells is stimulated by a differentiation factor. Differentiation factors may include one or a combination of any of the following:

BMP4
(SEQ ID NO: 33)
MIPGNRMLMV VLLCQVLLGG ASHASLIPET GKKKVAEIQG
HAGGRRSGQS HELLRDFEAT LLQMFGLRRR PQPSKSAVIP
DYMRDLYRLQ SGEEEEEQIH STGLEYPERP ASRANTVRSF
HHEEHLENIP GTSENSAFRF LFNLSSIPEN EVISSAELRL
FREQVDQGPD WERGFHRINI YEVMKPPAEV VPGHLITRLL
DTRLVHHNVT RWETFDVSPA VLRWTREKQP NYGLAIEVTH
LHQTRTHQGQ HVRISRSLPQ GSGNWAQLRP LLVTFGHDGR
GHALTRRRRA KRSPKHHSQR ARKKNKNCRR HSLYVDFSDV
GWNDWIVAPP GYQAFYCHGD CPFPLADHLN STNHAIVQTL
VNSVNSSIPK ACCVPTELSA ISMLYLDEYD KVVLKNYQEM
VVEGCGCR
FGF2
(SEQ ID NO: 34)
MVGVGGGDVE DVTPRPGGCQ ISGRGARGCN GIPGAAAWEA
ALPRRRPRRH PSVNPRSRAA GSPRTRGRRT EERPSGSRLG
DRGRGRALPG GRLGGRGRGR APERVGGRGR GRGTAAPRAA
PAARGSRPGP AGTMAAGSIT TLPALPEDGG SGAFPPGHFK
DPKRLYCKNG GFFLRIHPDG RVDGVREKSD PHIKLQLQAE
ERGVVSIKGV CANRYLAMKE DGRLLASKCV TDECFFFERL
ESNNYNTYRS RKYTSWYVAL KRTGQYKLGS KTGPGQKAIL
FLPMSAKS

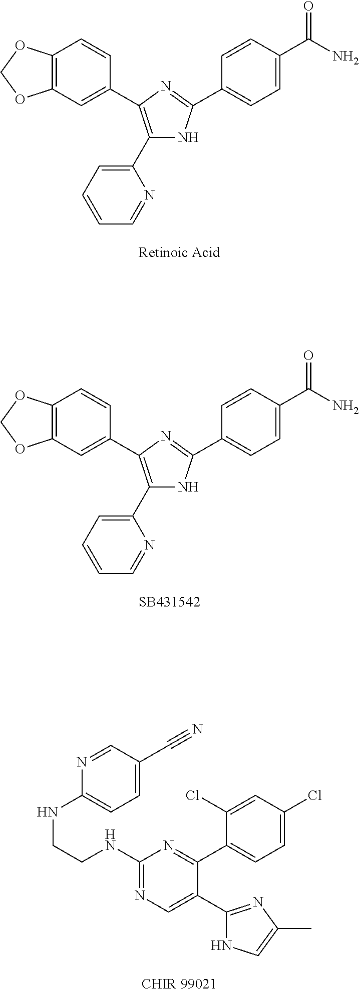 [SMILES: NC(=O)c1ccc(-c2nc(-c3ccc4c(c3)OCO4)c(-c3ccccn3)[nH]2)cc1 ; NC(=O)c1ccc(-c2nc(-c3ccc4c(c3)OCO4)c(-c3ccccn3)[nH]2)cc1 ; Cc1c[nH]c(-c2cnc(NCCNc3ccc(C#N)cn3)nc2-c2ccc(Cl)cc2Cl)n1]

In any of the methods or systems disclosed herein, the differentiation factors used may be functional fragments or variants of the polypeptides disclosed above with at least about 70% sequence identity to the above sequences. In any of the methods or systems disclosed herein, the differentiation factors used may be functional fragments or variants of the polypeptides disclosed above with at least about 80% sequence identity to the above sequences. In any of the methods or systems disclosed herein, the differentiation factors used may be functional fragments or variants of the polypeptides disclosed above with at least about 85% sequence identity to the above sequences. In any of the methods or systems disclosed herein, the differentiation factors used may be functional fragments or variants of the polypeptides disclosed above with at least about 90% sequence identity to the above sequences. In any of the methods or systems disclosed herein, the differentiation factors used may be functional fragments or variants of the polypeptides disclosed above with at least about 95% sequence identity to the above sequences. In any of the methods or systems disclosed herein, the differentiation factors used may be functional analogues of the small molecules disclosed above. The methods of the disclosure relate to the sequential exposure of a culture of cells to two or more different tissue culture mediums. In some embodiments, the methods relate to the sequential exposure of cells of the present disclosure to Cocktail Me or the tissue culture medium described herein.

The term “two-dimensional culture” as used herein is defined as cultures of cells that lie flat on hydrogels, including Matrigel® and vitronectin, disposed in culture vessels with only a one to four cell height. In some embodiments, two-dimensional culture is not more than 3 cells high. In some embodiments, two-dimensional culture is not more than 2 cells high. In some embodiments, two-dimensional culture is not more than 1 cell high.

As used herein, a “three-dimensional culture” is defined as a culture of cells that take a three dimensional shape while in culture. In some embodiments, the three-dimensional cultures are more than 1, 2, 3, 4, 5, 6, 7, 8, 9, 10 or more cells in height. In some embodiments, the three-dimensional culture has an organized shape self-assembled by way of simple culturing methods. In some embodiments, the three-dimensional culture comprises one or a plurality of spheroids or ganglioids.

As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes.

A spheroid of the present disclosure can have any suitable width, length, thickness, and/or diameter. In some embodiments, a spheroid may have a width, length, thickness, and/or diameter in a range from about 10 μm to about 50,000 μm, or any range therein, such as, but not limited to, from about 100 μm to about 200 μm, from about 100 μm to about 300 μm, from about 100 μm to about 400 μm, from about 100 μm to about 500 μm, from about 100 μm to about 600 μm, from about 100 μm to about 700 μm, about 50 μm to about 200 μm, from about 50 μm to about 250 μm, from about 100 μm to about 700 μm, about 300 μm to about 600 μm, about 400 μm to about 500 μm, about 500 μm to about 1,000 μm, about 600 μm to about 1,000 μm, about 700 μm to about 1,000 μm, about 800 μm to about 1,000 μm, about 900 μm to about 1,000 μm, about 750 μm to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 μm. In some embodiments, a spheroid may have a width, length, thickness, and/or diameter of about 50 μm, 100 μm, 200 μm, 300 μm, 400 μm, 500 μm, 600 μm, 700 μm, 800 μm, 900 μm, 1,000 μm, 5,000 μm, 10,000 μm, 20,000 μm, 30,000 μm, 40,000 μm, or 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above. In some embodiments, a spheroid of the present disclosure comprises enteric neurons, enteric glial cells, progenitor cells, epithelial cells, mesenchymal cells, smooth muscle cells, and RPE cells. In some embodiments, the spheroid comprises mesenchymal cells, epithelial cells and enteric neurons but is free of smooth muscle cells and free of RPEs. In some embodiments, the spheroid comprises no less than about 10,000, about 15,000, about 20,000, about 25,000, about 30,000, about 35,000, about 40,000, about 45,000, about 50,000, or about 60,000 cells. In some embodiments, the spheroid comprises from about 25,000 to about 100,000 cells. In some embodiments, the spheroid comprises from about 35,000 to about 100,000 cells. In some embodiments, the spheroid comprises from about 45,000 to about 100,000 cells. In some embodiments, the spheroid comprises from about 55,000 to about 100,000 cells. In some embodiments, the spheroid comprises from about 75,000 to about 100,000 cells.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell. In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types.

In some embodiments, the spheroids comprise at least two types of cells. In some embodiments, the spheroids comprise neuronal cells and non-neuronal cells. In some embodiments, the spheroids comprise neuronal cells and astrocytes at a ratio of about 5:1, about 4:1, about 3:1, about 2:1 or about 1:1 of neuronal cells to astrocytes. In some embodiments, the spheroids comprise neuronal cells and non-neuronal cells at a ratio of about 5:1, 4:1, 3:1, 2:1 or 1:1. In some embodiments, the spheroids comprise neuronal cells and non-neuronal cells at a ratio of about 1:5: 1:4, 1:3, or 1:2. Any combination of cell types disclosed herein may be used in the above-identified ratios within the spheroids of the disclosure.

Depending on the particular embodiment, groups of cells may be placed according to any suitable shape, geometry, and/or pattern. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures. In some embodiments, the spheroid is a “crestosphere,” which means that it comprises one or a plurality of neural crest cells identified in the specification. In some embodiments, the crestosphere comprises over about 50% of the neural crest cells relative to the total number of cells in the spheroid, over about 60% of the neural crest cells relative to the total number of cells in the spheroid, over about 70% of the neural crest cells relative to the total number of cells in the spheroid, over about 80% of the neural crest cells relative to the total number of cells in the spheroid, over about 90% of the neural crest cells relative to the total number of cells in the spheroid, over about 95% of the neural crest cells relative to the total number of cells in the spheroid, over about 30% of the neural crest cells, over about 40% of the neural crest cells relative to the total number of cells in the spheroid, over about 10% of the neural crest cells relative to the total number of cells in the spheroid, over about 20% of the neural crest cells relative to the total number of cells in the spheroid, over about 25% of the neural crest cells relative to the total number of cells in the spheroid, over about 30% of the neural crest cells relative to the total number of cells in the spheroid, over about 35% of the neural crest cells relative to the total number of cells in the spheroid, over about 45% of the neural crest cells relative to the total number of cells in the spheroid, over about 55% of the neural crest cells relative to the total number of cells in the spheroid.

The term “subject” as used herein refers to any animal (e.g., a mammal), including, but not limited to, humans, non-human primates, canines, felines, rodents, and the like. Preferably, the subject is a human subject. The terms “subject,” “individual,” and “patient” are used interchangeably herein. The terms “subject,” “individual,” and “patient” thus encompass individuals having disorders of the gut-brain interaction (e.g., Achalasia, Hirschsprung's disease, Intestinal pseudo-obstruction, Gastroesophageal reflux disease (GERD), Functional dysphagia, Functional dyspepsia, Irritable bowel syndrome (IBS), Gastroparesis, Functional constipations, Functional Diarrhea, and Fecal incontinence).

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a gut motility. In some embodiments, the activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue. In some embodiments, an effective amount is that amount of a substance need to confer a biological effect such as differentiation of a cell in response to exposure to PDGFR or a PDGFR inhibitor disclosed herein.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

The term “preventing” or “prevention” or “prevent” as used herein refers to prophylactic or preventative measures that prevent or slow the development of a targeted pathologic condition or disorder. Those in need of treatment include those already diagnosed with the disorder; those prone to have the disorder; and those in whom the disorder is to be prevented.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder associated with PDGFR activity such as, for example, a gut motility disorder, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

In some embodiments, the composition, spheroid or ganglioids is administered at a desired dosage, which in some aspects includes a desired dose or number of cells and/or a desired ratio of neuronal cell subpopulations. Thus, the dosage of cells In some embodiments, is based on a total number of cells (or number per m² body surface area or per kg body weight) and a desired ratio of the individual populations or sub-types. In some embodiments, the dosage of cells is based on a desired total number (or number per m² body surface area or per kg of body weight) of cells in the individual populations or of individual cell types. In some embodiments, the dosage is based on a combination of such features, such as a desired number of total cells, desired ratio, and desired total number of cells in the individual populations.

In some embodiments, the composition, spheroid or ganglioids is administered at or within a tolerated difference of a desired dose of total cells, such as a desired dose of subtypes of neuronal cells, e.g., enteric neurons, glial cells and mesenchymal cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual neuronal subpopulations. Thus, in some embodiments, the dosage is based on a desired fixed or minimum dose of neuronal subpopulations and a desired ratio thereof.

In certain embodiments, composition, spheroid or ganglioids is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual neuronal subpopulations of cells is within a range of between at or about 10⁴ and at or about 10⁹ cells/meter² (m²) body surface area, such as between 105 and 10⁶ cells/m² body surface area, for example, at or about 1 ×10⁵ cells/m², 1.5 ×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ neuronal cells/meter² (m²) body surface area, such as between 10⁵ and 10⁶ neuronal or glial cells/m² body surface area, for example, at or about 1×10⁵ neuronal or glial cells/m², 1.5×10⁵ neuronal or glial cells/m², 2×10⁵ neuronal or glial cells/m², or 1×10⁶ neuronal or glial cells/m² body surface area.

In some embodiments, the cells are administered at or within a certain range of error of between at or about 10⁴ and at or about 10⁹ cells/meter² (m²) body weight, such as between 10⁵ and 10⁶ cells/m² body weight, for example, at or about 1×10⁵ cells/m², 1.5 ×10⁵ cells/m², 2×10⁵ cells/kg, or 1×10⁶ cells/m² body surface area.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or 0.1% from the specified value, as such variations are appropriate to perform the disclosed methods.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived. In some embodiments, the functional fragment may retain 95%, 94%, 93%, 92%, 91%, 90%, 85%, 80%, 75%, 70%, 65%, or 60% sequence identity to the amino acid sequence encoded by any of the mRNA sequences of Table 2.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids. [00279]“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

“Optional” or “optionally” means that the subsequently described event, circumstance, or material may or may not occur or be present, and that the description includes instances where the event, circumstance, or material occurs or is present and instances where it does not occur or is not present.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment. In some cases the subject is an experimental model, such as a mouse.

The term “associated” or “associated with” in the context of a substance or substance activity or function associated with a disease (e.g., a protein associated disease, a symptom associated with a gut motility disorder, a symptom associated with NO neuron activity) means that the disease (e.g., the gut motility disorder) is caused by (in whole or in part), or a symptom of the disease is caused by (in whole or in part) the substance or substance activity or function. For example, a symptom of a gut motility disease or condition may be a symptom that results (entirely or partially) from modulation of NO neuron activity (e.g., induction of colonic motility). As used herein, what is described as being associated with a disease, if a causative agent, could be a target for treatment of the disease. For example, a gut motility disorder, may be treated with an agent (e.g., compound as described herein) effective for modulating NO neuron activity (e.g., effective for inducing colonic motility).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a cell (e.g., an enteric neuron or enteric glial cell or crestosphere comprising one or both of the same). In some embodiments contacting includes allowing a compound described herein to interact with a protein or enzyme that is involved in a signaling pathway, such as PDGFR.

As defined herein, the term “inhibition,” “inhibit,” “inhibiting,” and the like in reference to a protein-inhibitor (e.g., antagonist) interaction means negatively affecting (e.g., decreasing) the activity or function of the protein (e.g., PDGFR) relative to the activity or function of the protein in the absence of the inhibitor (e.g., a compound as described herein). In some embodiments inhibition refers to reduction of a disease or symptoms of disease (e.g., a gut motility disorder). In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs.

The enteric neurons and/or enteric glial cells of the disclosure can be administered alone or can be coadministered to the patient. In some embodiments, coadministration is completed with the enteric neurons or glial cells in a ganglioid or spheroid structure. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intravenous injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995). In some embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The disclosure relates to pharmaceutical compositions comprising any enteric neuronal cell or glial cell disclosed herein; and a pharmaceutically acceptable carrier. The actual amount effective for a particular application will depend, inter alia, on the condition being treated. When administered in methods to treat a disease, such compositions will contain an amount of active ingredient effective to achieve the desired result, e.g., modulating the activity of a subject (e.g., increase the number of intergenic neurons in the subject), and/or reducing, eliminating, or slowing the progression of disease symptoms (e.g., symptoms of a gut motility disorder). Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of a gut motility disorder), kind of concurrent treatment, complications from the disease being treated or other health-related problems. Other therapeutic regimens or agents can be used in conjunction with the methods and compounds of Applicants' disclosure, filed as 63/296,15, on Jan. 3, 2022, which is incorporated by reference in its entirety. Adjustment and manipulation of established dosages (e.g., frequency and duration) are well within the ability of those skilled in the art.

In some embodiments, the compositions are administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the by the particular method used to administer the cell composition. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

In some embodiments, the pharmaceutical composition comprises the TVM or VM composition in an amount that is effective to treat or prevent the disease or condition, such as a therapeutically effective or prophylactically effective amount. Thus, in some embodiments, the methods of administration include administration of the composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the pharmaceutical composition is administered at a desired dosage, which in some aspects includes a desired dose or number of cells and/or a desired number of enteric cell and/or glial cell subpopulations. Thus, the dosage of cells in some embodiments is based on a total number of cells (or number per m2 body surface area or per kg body weight) and a desired amount of the individual populations or sub-types. In some embodiments, the dosage of cells is based on a desired total number (or number per m2 body surface area or per kg of body weight) of cells in the individual populations or of individual cell types. In some embodiments, the dosage is based on a combination of such features, such as a desired number of total cells, and desired total number of cells in the individual populations.

In some embodiments, the pharmaceutical composition is administered at or within a tolerated difference of a desired dose of total cells, such as a desired dose of spheroids, gangiloids, enteric neuronal cells and/or glial cells. In some aspects, the desired dose is a desired number of cells, a desired number of cells per unit of body surface area or a desired number of cells per unit of body weight of the subject to whom the cells are administered, e.g., cells/m2 or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m2 or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and/or based on a desired fixed dose of two or more, e.g., each, of the enteric neuronal cells and glial cell subpopulations. Thus, in some embodiments, the dosage is based on a desired fixed or minimum dose of glial cell subpopulations and a desired ratio thereof.

### Compositions

The present disclosure relates to a spheroid comprising a plurality of cell types, including, but not limited to, enteric neurons. In some embodiments, the spheroid further comprises enteric glial cells. In some embodiments, the spheroid further comprises progenitor cells. In some embodiments, the spheroid further comprises epithelial cells. In some embodiments, the spheroid further comprises mesenchymal cells. In some embodiments, the spheroid further comprises smooth muscle cells. In some embodiments, the spheroid further comprises retinal pigmented epithelial (RPE) cells.

The present disclosure relates to a spheroid comprising a plurality of cell types, wherein the cell types comprise at least about 5% enteric neurons. In some embodiments, the spheroid comprises at least about 10% enteric neurons. In some embodiments, the spheroid comprises at least about 15% enteric neurons. In some embodiments, the spheroid comprises at least about 20% enteric neurons. In some embodiments, the spheroid comprises at least about 25% enteric neurons. In some embodiments, the spheroid comprises at least about 30% enteric neurons. In some embodiments, the spheroid comprises at least about 35% enteric neurons. In some embodiments, the spheroid comprises at least about 40% enteric neurons. In some embodiments, the spheroid comprises at least about 45% enteric neurons. In some embodiments, the spheroid comprises at least about 50% enteric neurons. In some embodiments, the spheroid comprises at least about 55% enteric neurons. In some embodiments, the spheroid comprises at least about 60% enteric neurons. In some embodiments, the spheroid comprises at least about 65% enteric neurons. In some embodiments, the spheroid comprises at least about 70% enteric neurons. In some embodiments, the spheroid comprises at least about 75% enteric neurons. In some embodiments, the spheroid comprises at least about 80% enteric neurons. In some embodiments, the spheroid comprises at least about 85% enteric neurons. In some embodiments, the spheroid comprises at least about 90% enteric neurons.

In some embodiments, the spheroid comprises from about 5% to about 90% enteric neurons. In some embodiments, the spheroid comprises from about 10% to about 90% enteric neurons. In some embodiments, the spheroid comprises from about 15% to about 90% enteric neurons. In some embodiments, the spheroid comprises from about 20% to about 90% enteric neurons. In some embodiments, the spheroid comprises from about 25% to about 90% enteric neurons. In some embodiments, the spheroid comprises from about 30% to about 90% enteric neurons. In some embodiments, the spheroid comprises from about 35% to about 90% enteric neurons. In some embodiments, the spheroid comprises from about 40% to about 90% enteric neurons. In some embodiments, the spheroid comprises from about 45% to about 90% enteric neurons. In some embodiments, the spheroid comprises from about 50% to about 90% enteric neurons. In some embodiments, the spheroid comprises from about 55% to about 90% enteric neurons. In some embodiments, the spheroid comprises from about 60% to about 90% enteric neurons. In some embodiments, the spheroid comprises from about 65% to about 90% enteric neurons. In some embodiments, the spheroid comprises from about 70% to about 90% enteric neurons. In some embodiments, the spheroid comprises from about 75% to about 90% enteric neurons. In some embodiments, the spheroid comprises from about 80% to about 90% enteric neurons. In some embodiments, the spheroid comprises from about 85% to about 90% enteric neurons.

The present disclosure relates to a spheroid comprising a plurality of cell types, wherein the cell types comprise at least about 5% enteric glial cells. In some embodiments, the spheroid comprises at least about 10% enteric glial cells. In some embodiments, the spheroid comprises at least about 15% enteric glial cells. In some embodiments, the spheroid comprises at least about 20% enteric glial cells. In some embodiments, the spheroid comprises at least about 25% enteric glial cells. In some embodiments, the spheroid comprises at least about 30% enteric glial cells. In some embodiments, the spheroid comprises at least about 35% enteric glial cells. In some embodiments, the spheroid comprises at least about 40% enteric glial cells. In some embodiments, the spheroid comprises at least about 45% enteric glial cells. In some embodiments, the spheroid comprises at least about 50% enteric glial cells. In some embodiments, the spheroid comprises at least about 55% enteric glial cells. In some embodiments, the spheroid comprises at least about 60% enteric glial cells. In some embodiments, the spheroid comprises at least about 65% enteric glial cells. In some embodiments, the spheroid comprises at least about 70% enteric glial cells. In some embodiments, the spheroid comprises at least about 75% enteric glial cells. In some embodiments, the spheroid comprises at least about 80% enteric glial cells. In some embodiments, the spheroid comprises at least about 85% enteric glial cells. In some embodiments, the spheroid comprises at least about 90% enteric glial cells.

In some embodiments, the spheroid comprises from about 0% to about 90% enteric glial cells. In some embodiments, the spheroid comprises from about 5% to about 90% enteric glial cells. In some embodiments, the spheroid comprises from about 10% to about 90% enteric glial cells. In some embodiments, the spheroid comprises from about 15% to about 90% enteric glial cells. In some embodiments, the spheroid comprises from about 20% to about 90% enteric glial cells. In some embodiments, the spheroid comprises from about 25% to about 90% enteric glial cells. In some embodiments, the spheroid comprises from about 30% to about 90% enteric glial cells. In some embodiments, the spheroid comprises from about 35% to about 90% enteric glial cells. In some embodiments, the spheroid comprises from about 40% to about 90% enteric glial cells. In some embodiments, the spheroid comprises from about 45% to about 90% enteric glial cells. In some embodiments, the spheroid comprises from about 50% to about 90% enteric glial cells. In some embodiments, the spheroid comprises from about 55% to about 90% enteric glial cells. In some embodiments, the spheroid comprises from about 60% to about 90% enteric glial cells. In some embodiments, the spheroid comprises from about 65% to about 90% enteric glial cells. In some embodiments, the spheroid comprises from about 70% to about 90% enteric glial cells. In some embodiments, the spheroid comprises from about 75% to about 90% enteric glial cells. In some embodiments, the spheroid comprises from about 80% to about 90% enteric glial cells. In some embodiments, the spheroid comprises from about 85% to about 90% enteric glial cells.

The present disclosure relates to a spheroid comprising a plurality of cell types, where the cell types comprise at least about 5% progenitor cells. In some embodiments, the spheroid comprises least about 10% progenitor cells. In some embodiments, the spheroid comprises least about 15% progenitor cells. In some embodiments, the spheroid comprises least about 20% progenitor cells. In some embodiments, the spheroid comprises least about 25% progenitor cells. In some embodiments, the spheroid comprises least about 30% progenitor cells. In some embodiments, the spheroid comprises least about 35% progenitor cells. In some embodiments, the spheroid comprises least about 40% progenitor cells.

In some embodiments, the spheroid comprises from about 0% to about 40% progenitor cells. In some embodiments, the spheroid comprises from about 5% to about 40% progenitor cells. In some embodiments, the spheroid comprises from about 10% to about 40% progenitor cells. In some embodiments, the spheroid comprises from about 15% to about 40% progenitor cells. In some embodiments, the spheroid comprises from about 20% to about 40% progenitor cells. In some embodiments, the spheroid comprises from about 25% to about 40% progenitor cells. In some embodiments, the spheroid comprises from about 30% to about 40% progenitor cells. In some embodiments, the spheroid comprises from about 35% to about 40% progenitor cells.

The present disclosure relates to a spheroid comprising a plurality of cell types, where the cell types comprise at least about 5% epithelial cells. In some embodiments, the spheroid comprises least about 10% epithelial cells. In some embodiments, the spheroid comprises least about 15% epithelial cells. In some embodiments, the spheroid comprises least about 20% epithelial cells.

In some embodiments, the spheroid comprises from about 0% to about 20% epithelial cells. In some embodiments, the spheroid comprises from about 5% to about 20% epithelial cells. In some embodiments, the spheroid comprises from about 10% to about 20% epithelial cells. In some embodiments, the spheroid comprises from about 15% to about 20% epithelial cells.

The present disclosure relates to a spheroid comprising a plurality of cell types, where the cell types comprise at least about 5% mesenchymal cells. In some embodiments, the spheroid comprises least about 10% mesenchymal cells. In some embodiments, the spheroid comprises least about 15% mesenchymal cells. In some embodiments, the spheroid comprises least about 20% mesenchymal cells. In some embodiments, the spheroid comprises least about 25% mesenchymal cells. In some embodiments, the spheroid comprises least about 30% mesenchymal cells. In some embodiments, the spheroid comprises least about 35% mesenchymal cells. In some embodiments, the spheroid comprises least about 40% mesenchymal cells. In some embodiments, the spheroid comprises least about 45% mesenchymal cells. In some embodiments, the spheroid comprises least about 50% mesenchymal cells. In some embodiments, the spheroid comprises least about 55% mesenchymal cells. In some embodiments, the spheroid comprises least about 60% mesenchymal cells. In some embodiments, the spheroid comprises least about 70% mesenchymal cells.

In some embodiments, the spheroid comprises from about 0% to about 70% mesenchymal cells. In some embodiments, the spheroid comprises from about 5% to about 70% mesenchymal cells. In some embodiments, the spheroid comprises from about 10% to about 70% mesenchymal cells. In some embodiments, the spheroid comprises from about 15% to about 70% mesenchymal cells. In some embodiments, the spheroid comprises from about 20% to about 70% mesenchymal cells. In some embodiments, the spheroid comprises from about 25% to about 70% mesenchymal cells. In some embodiments, the spheroid comprises from about 30% to about 70% mesenchymal cells. In some embodiments, the spheroid comprises from about 35% to about 70% mesenchymal cells. In some embodiments, the spheroid comprises from about 40% to about 70% mesenchymal cells. In some embodiments, the spheroid comprises from about 45% to about 70% mesenchymal cells. In some embodiments, the spheroid comprises from about 50% to about 70% mesenchymal cells. In some embodiments, the spheroid comprises from about 55% to about 70% mesenchymal cells. In some embodiments, the spheroid comprises from about 60% to about 70% mesenchymal cells. In some embodiments, the spheroid comprises from about 65% to about 70% mesenchymal cells.

The present disclosure relates to a spheroid comprising a plurality of cell types, where the cell types comprise at least about 5% smooth muscle cells. In some embodiments, the spheroid comprises least about 10% smooth muscle cells. In some embodiments, the spheroid comprises least about 15% smooth muscle cells. In some embodiments, the spheroid comprises least about 20% smooth muscle cells. In some embodiments, the spheroid comprises least about 25% smooth muscle cells. In some embodiments, the spheroid comprises least about 30% smooth muscle cells.

In some embodiments, the spheroid comprises from about 0% to about 30% smooth muscle cells. In some embodiments, the spheroid comprises from about 5% to about 30% smooth muscle cells. In some embodiments, the spheroid comprises from about 10% to about 30% smooth muscle cells. In some embodiments, the spheroid comprises from about 15% to about 30% smooth muscle cells. In some embodiments, the spheroid comprises from about 20% to about 30% smooth muscle cells. In some embodiments, the spheroid comprises from about 25% to about 30% smooth muscle cells.

The present disclosure relates to a spheroid comprising a plurality of cell types, where the cell types comprise at least about 5% RPE cells. In some embodiments, the spheroid comprises least about 10% RPE cells. In some embodiments, the spheroid comprises least about 15% RPE cells. In some embodiments, the spheroid comprises least about 20% RPE cells.

In some embodiments, the spheroid comprises from about 0% to about 30% RPE cells. In some embodiments, the spheroid comprises from about 5% to about 30% RPE cells. In some embodiments, the spheroid comprises from about 10% to about 30% RPE cells. In some embodiments, the spheroid comprises from about 15% to about 30% RPE cells. In some embodiments, the spheroid comprises from about 20% to about 30% RPE cells. In some embodiments, the spheroid comprises from about 25% to about 30% RPE cells.

In some embodiments, the spheroid is substantially free of or free of retinal pigmented epithelial cells. In some embodiments, the spheroid is substantially free of or free of epithelial cells. In some embodiments, the spheroid is substantially free of or free of smooth muscle cells. In some embodiments, the spheroid is substantially free of or free of mesenchymal cells. In some embodiments, the spheroid is substantially free of or free of non-neuronal cells. In some embodiments, the spheroid is substantially free of or free of enteric glia. In some embodiments, the spheroid is substantially free of or free of a progenitor cell. In some embodiments, the spheroid is free or substantially free of RPEs.

In some embodiments, the spheroid comprises from about 25% to about 60% enteric neurons and from about 25% to about 60% progenitor cells. In some embodiments, the spheroid comprises from about 30% to about 60% enteric neurons and from about 30% to about 60% progenitor cells. In some embodiments, the spheroid comprises from about 35% to about 60% enteric neurons and from about 35% to about 60% progenitor cells. In some embodiments, the spheroid comprises from about 40% to about 60% enteric neurons and from about 40% to about progenitor cells.

In some embodiments, the spheroid comprises from about 10% to about 25% enteric neurons and from about 10% to about 35% glia cells. In some embodiments, the spheroid comprises from about 15% to about 25% enteric neurons and from about 10% to about 35% glia cells.

In some embodiments, the spheroid comprises the percentages of cell types found in Table 1.

TABLE 1

| | Approximate | |
|---|---|---|
| | Percentage | |
| | relative to | |
| CELL TYPE | all cells | Embodiments of species |
| Cell Composition 1 | | |
| Unknown | About 5% | Human, canine, feline, |
| | | porcine, non-human primate |
| Epithelial | About 5% | Human, canine, feline, |
| | | porcine, non-human primate |
| Mesenchymal | About 10% | Human, canine, feline, |
| | | porcine, non-human primate |
| Progenitor | About 40% | Human, canine, feline, |
| | | porcine, non-human primate |
| Enteric Neural Cells | About 40% | Human, canine, feline, |
| | | porcine, non-human primate |
| Cell Composition 2 | | |
| Unknown | About 5% | Human, canine, feline, |
| | | porcine, non-human primate |
| Epithelial | About 5% | Human, canine, feline, |
| | | porcine, non-human primate |
| Mesenchymal | About 10% | Human, canine, feline, |
| | | porcine, non-human primate |
| Induced pluripotent | About 40% | Human, canine, feline, |
| cells | | porcine, non-human primate |
| Enteric Neural Cells | About 40% | Human, canine, feline, |
| | | porcine, non-human primate |
| Cell Composition 3 | | |
| Unknown | About 5% | Human, canine, feline, |
| | | porcine, non-human primate |
| Epithelial | About 5% | Human, canine, feline, |
| | | porcine, non-human primate |
| Mesenchymal | About 10% | Human, canine, feline, |
| | | porcine, non-human primate |
| Neural Crest Cells | About 40% | Human, canine, feline, |
| | | porcine, non-human primate |
| Enteric Neural Cells | About 40% | Human, canine, feline, |
| | | porcine, non-human primate |

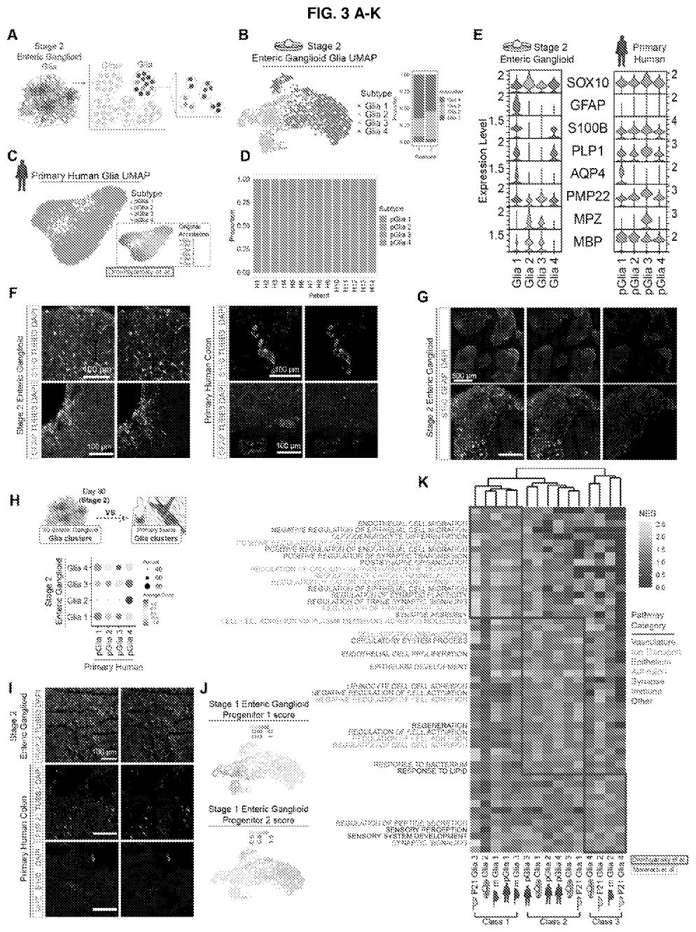

The present disclosure is related to a composition comprising a spheroid comprising enteric neurons, wherein the enteric neurons comprise SOX10 and CD24. The composition, in some embodiments, comprises cells expressing the biomarkers disclosed in FIG. 3E.

The present disclosure also relates to a system comprising: (i) a cell culture vessel optionally comprising a hydrogel; (ii) one or a plurality of stem cells or neural crest cells either in suspension or as a component of a spheroid; and (iii) on or plurality of differentiation factors.

In some embodiments, the system further comprises one or combination of culture mediums disclosed herein. The disclosure also relates to a method of culturing enteric neurons in a system, the system comprising: (i) a cell culture vessel optionally comprising a hydrogel; (ii) one or a plurality of stem cells or neural crest cells either in suspension or as a component of a spheroid; and (iii) on or plurality of differentiation factors. In some embodiments, the system further comprises one or combination of culture mediums disclosed herein. In some embodiments, the methods relate to replacing medium during a culture time of form about 12 to about 21 days at least one time to (i) expose one or a plurality of stem cells to a first cell medium for a time period sufficient to differentiate the one or plurality of stem cells into neural crest cells and the sequentially replacing the medium to (ii) expose one or plurality of neural crest cells to a second cell medium for a time period sufficient to differentiate the one or plurality of neural crest cells into enteric neurons.

### Methods

In some embodiments, compounds and compositions described herein are useful in treating a gut motility disorder. Thus, provided herein are methods of treating a gut motility disorder, comprising administering to a subject in need thereof, a therapeutically effective amount of a enteric neuron, enteric glial cell or spheroid comprising the same as described herein, or a composition comprising a enteric neuron, enteric glial cell or spheroid comprising the same. Disorders treatable by the present compounds and compositions include, e.g., achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

The disclosure relates to a method of transplanting a subject with one or a plurality of compositions herein comprising administering to the subject in need thereof a therapeutically effective amount of a enteric neuron, enteric glial cell or spheroid comprising the same as described herein, or a pharmaceutically acceptable salt thereof, or a composition comprising a enteric neuron, enteric glial cell or spheroid comprising the same. Disorders treatable by the present compounds and compositions include, e.g., achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence. In some embodiment, the subject is a rodent, such as a mouse. The disclosure therefore relates to a mammalian subject comprising the composition comprising a enteric neuron, enteric glial cell or spheroid comprising the same as disclosed herein.

The disclosure relates to a method of enriching a cell culture with a plurality of enteric neurons by exposing the cell culture with one or a plurality of PDGFR inhibitors. In some embodiments, the PDGFR inhibitors comprise an effective amount of a platelet-derived growth factor receptor (PDGFR) inhibitor or a pharmaceutically acceptable salt thereof. Examples of PDGFR inhibitors include, but are not limited to, (Z)-orantinib, AC710, AC710 mesylate, AG 1295, amuvatinib, amuvatinib hydrochloride, avapritinib, axitinib, AZD2932, cediranib, cediranib maleate, chiauranib, CHIR-124, CP-673451, crenolanib, dovitinib, dovitinib lactate, dovitinib lactate hydrate, dovitinib-D8, ENMD-2076, ENMD-2076 tartrate, flumatinib, flumatinib mesylate, GZD856, GZD856 formic, HG-7-85-01, hypothemycrin, ilorasertib, ilorasertib hydrochloride, imatinib, imatinib D4, imatinib D8, imatinib mesylate, JI-101, JNJ-10198409, KG5, Ki20227, lenvatinib, lenvatinib mesylate, linifanib, masitinib, masitinib mesylate, methylnissolin, multi-kinase inhibitor 1, N-(p-coumaroyl) serotonin, nintedanib, nintedanib esylate, NVP-ACC789, orantinib, pazopanib, pazopanib hydrochloride, PD-089828, PD-161570, PDGFRα kinase inhibitor 1, ponatinib, ponatinib D8, PP121, PP58, regorafenib, regorafenib D3, regorafenib hydrochloride, regorafenib monohydrate, ripretinib, sennoside B, seralutinib, SU5402, SU14813, SU14813 maleate, SU16f, SU4312, SU4984, sunitinib, sunitinib D10, sunitinib malate, sunitinib-d4, TAK-593, tandutinib, tandutinib hydrochloride, telatinib, telatinib mesylate, TG 100572, TG 100572 hydrochloride, TG 100801, TG 100801 hydrochloride, toceranib, toceranib phosphate, toceranib-d8, trapidil, tyrosine kinase-IN-1, tyrphostin AG1296, tryphostin AG1433, and vorolanib.

In further embodiments, the PDGFR inhibitor is selected from:

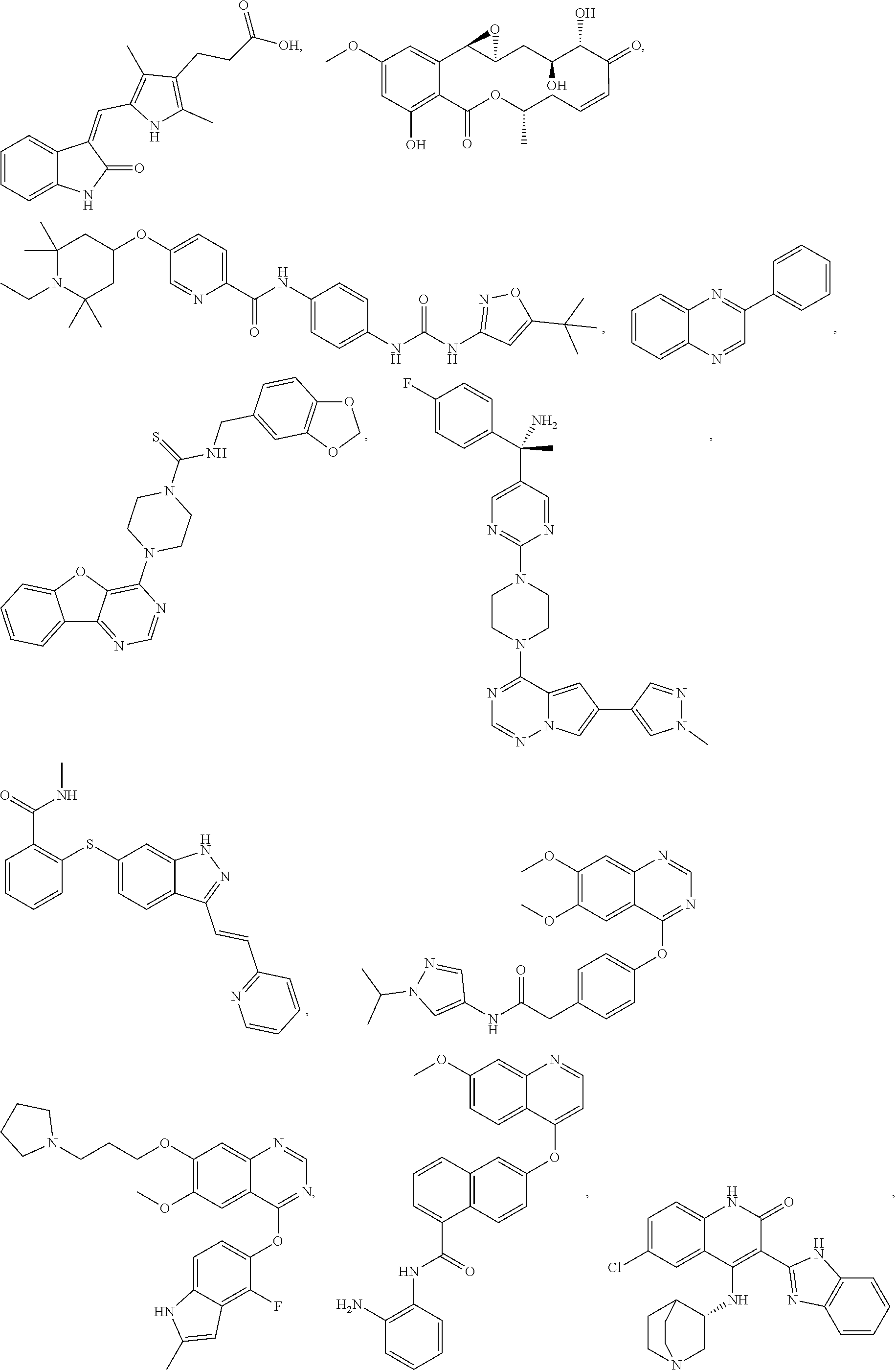

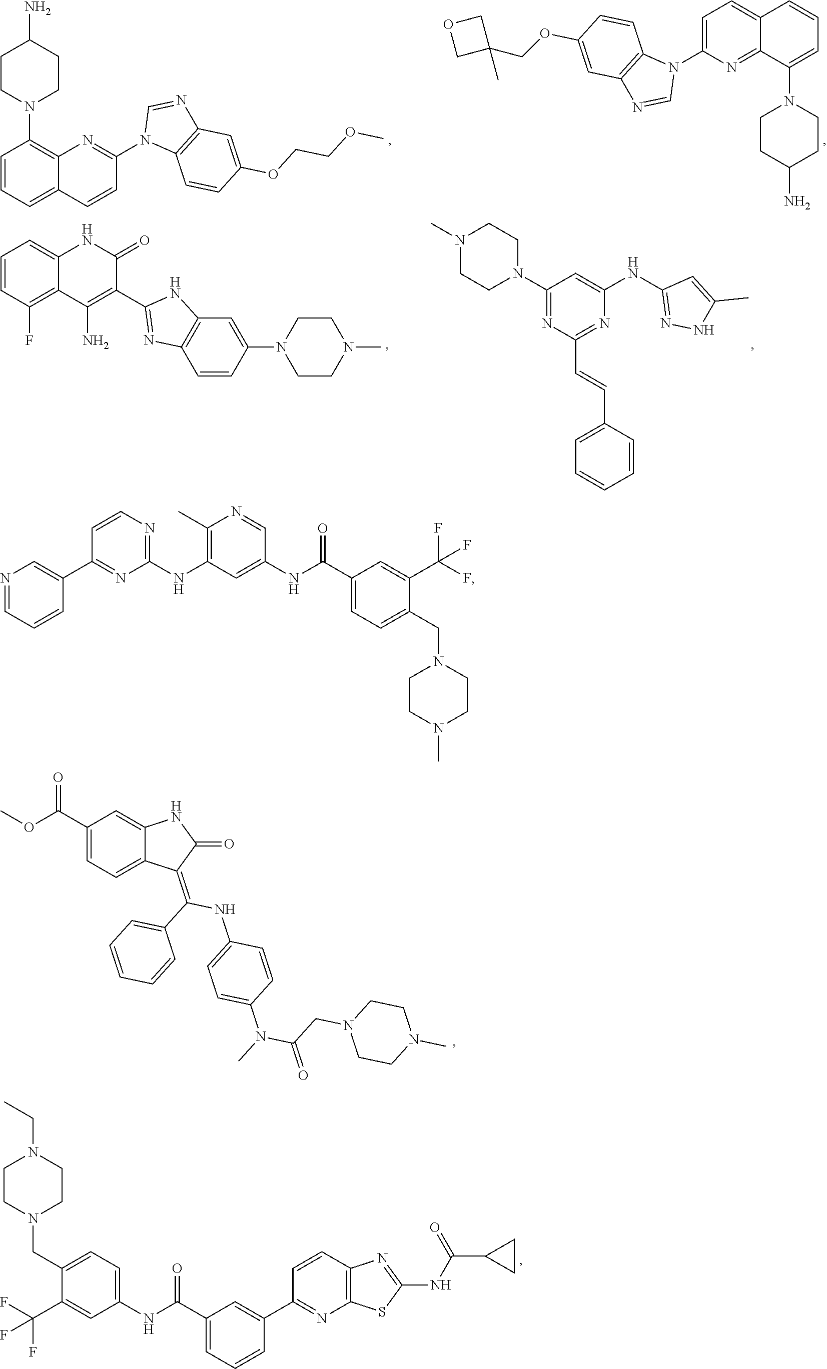

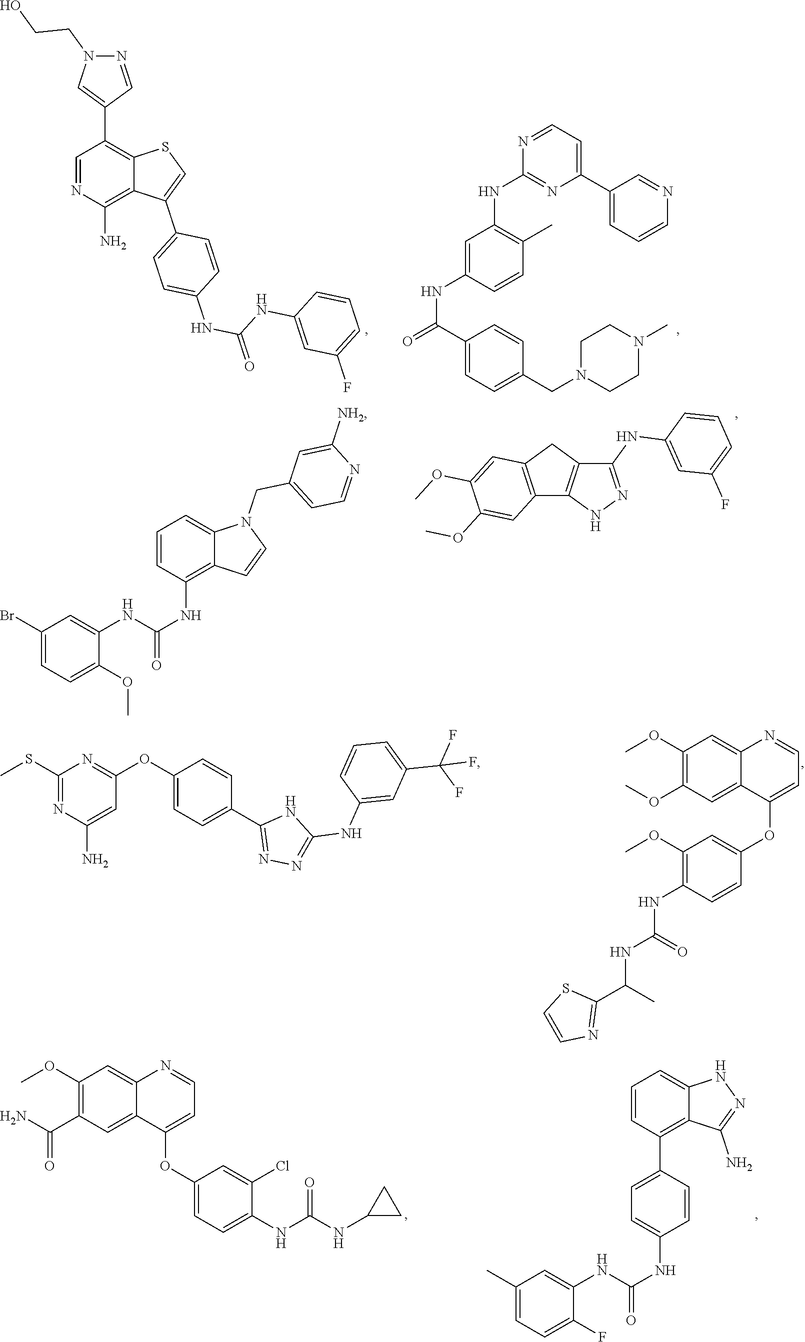

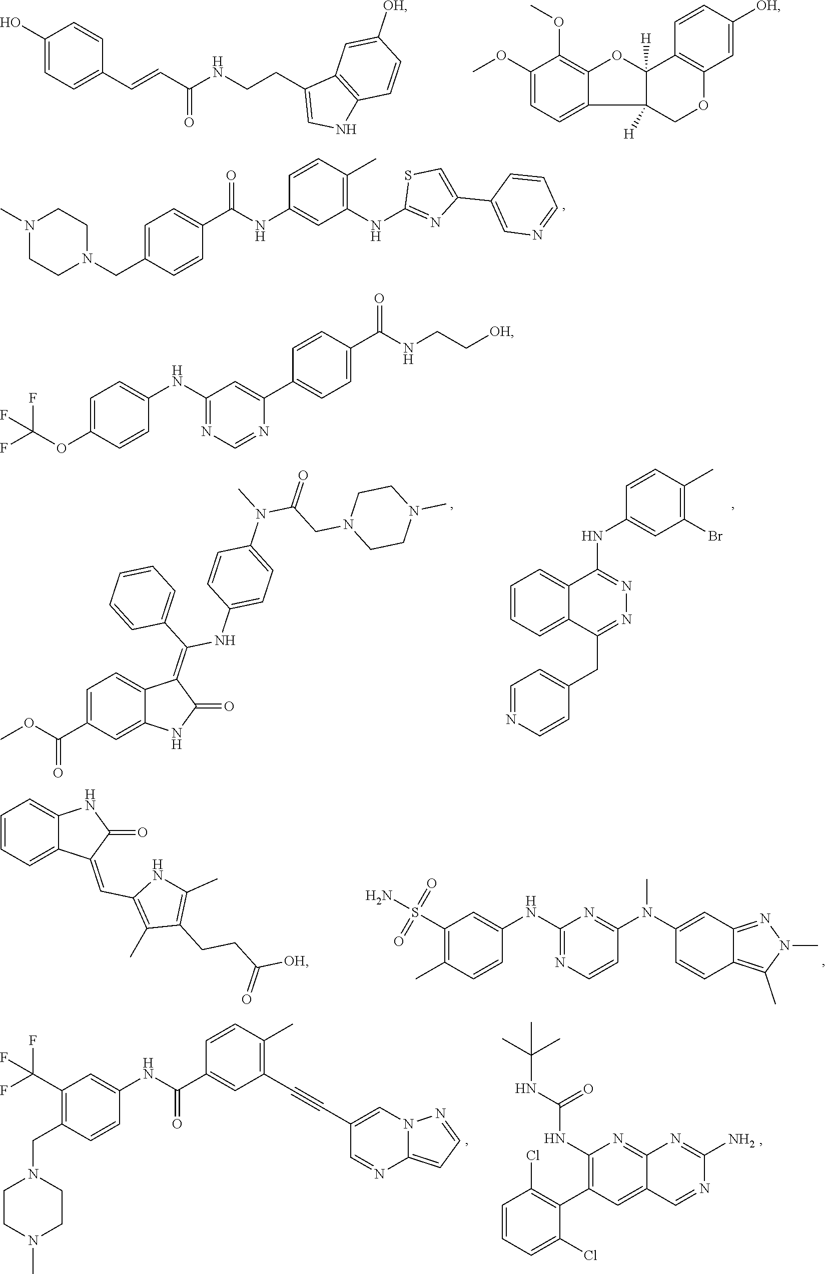

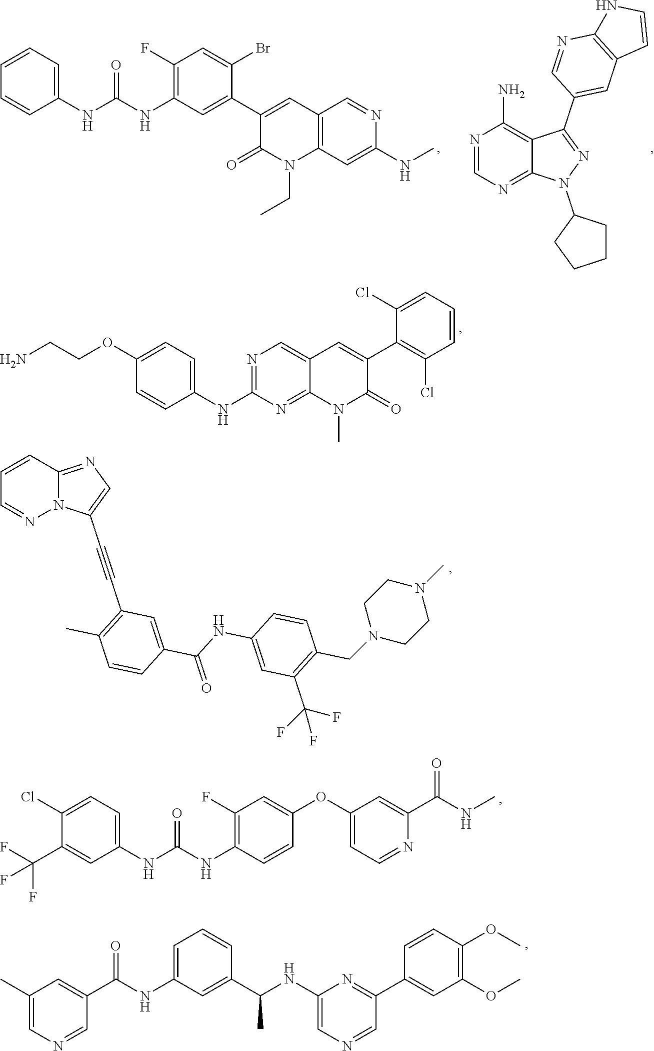

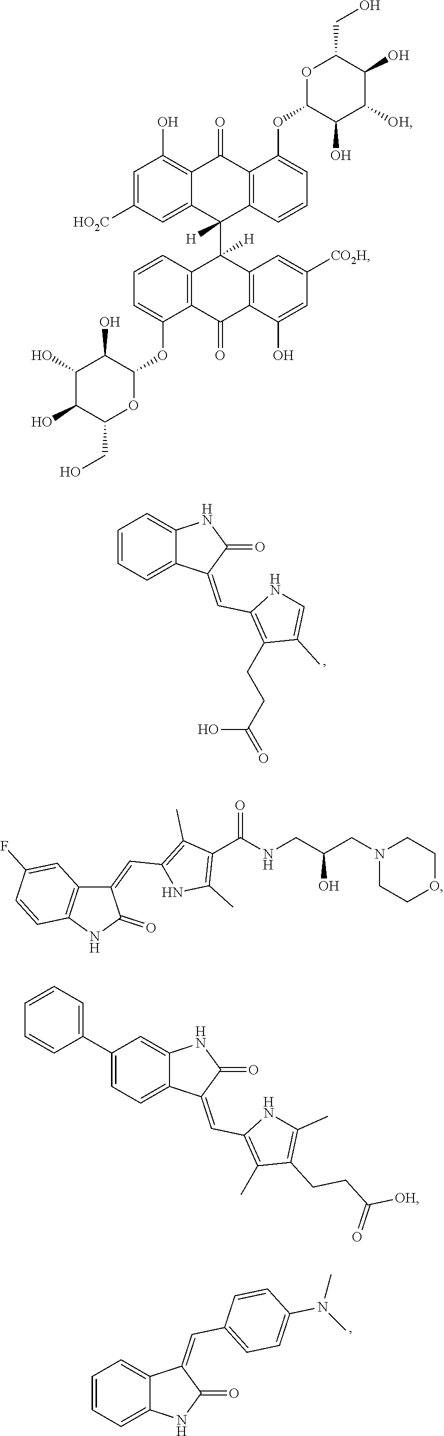

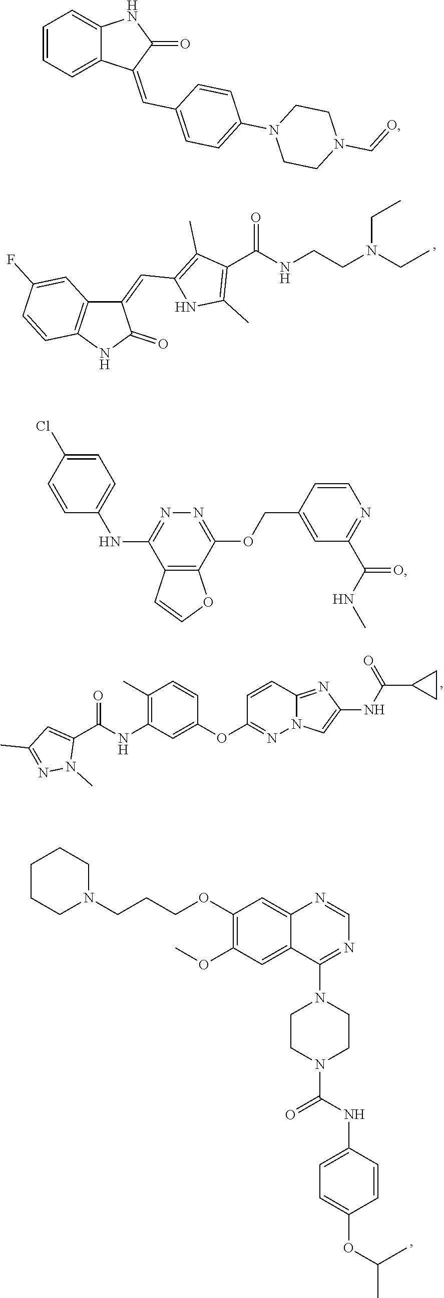

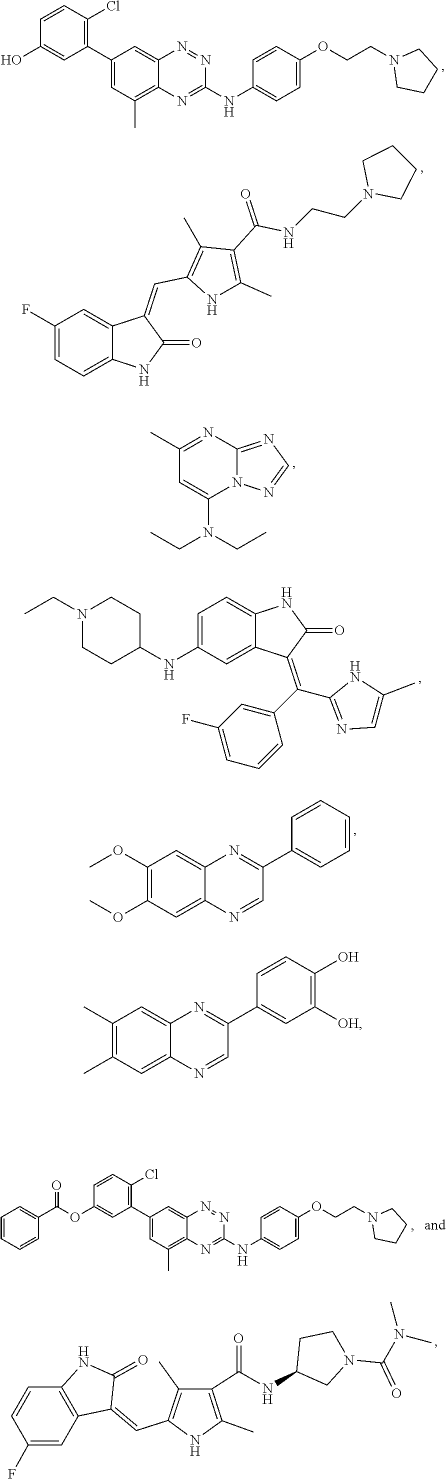

or a pharmaceutically acceptable salt thereof.

In further embodiments, the PDGFR inhibitor is a hydrate. In still further embodiments, the PDGFR inhibitor is selected from:

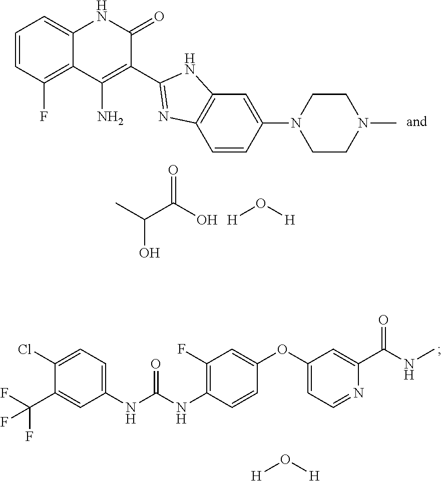

In further embodiments, the PDGFR inhibitor is an isotope. In still further embodiments, the PDGFR inhibitor is deuterated. In yet further embodiments, the PDGFR inhibitor is selected from:

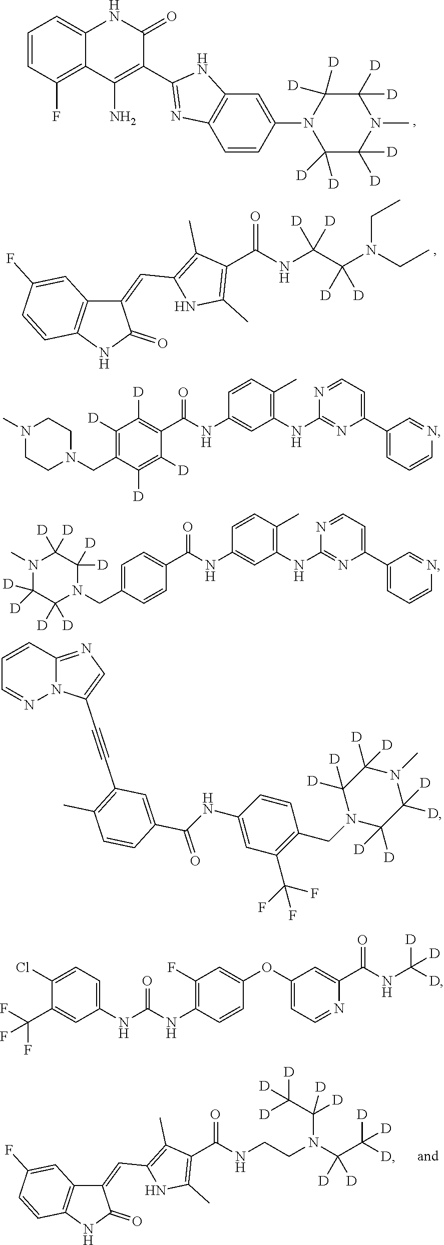

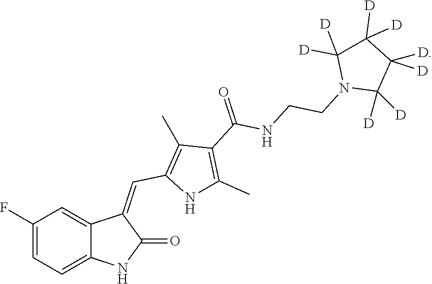

or a pharmaceutically acceptable salt thereof.

In further embodiments, the PDGFR inhibitor is administered as a pharmaceutically acceptable salt. Pharmaceutically acceptable salts include, but are not limited to, mesylates, hydrochlorides, maleates, lactates, tartrates, formates, esylates, phosphates, or malates. In still further embodiments, the pharmaceutically acceptable salt has a structure selected from:

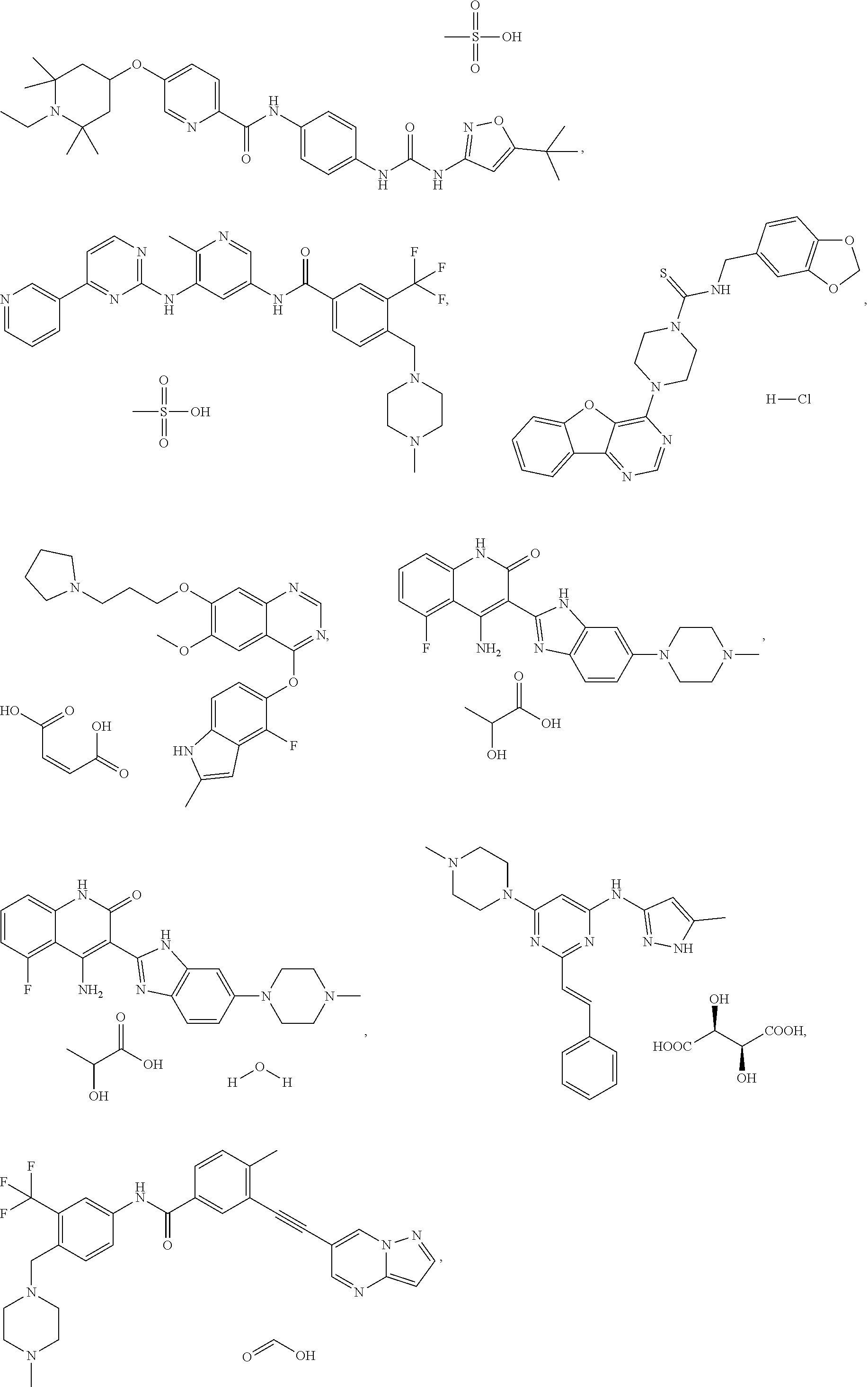

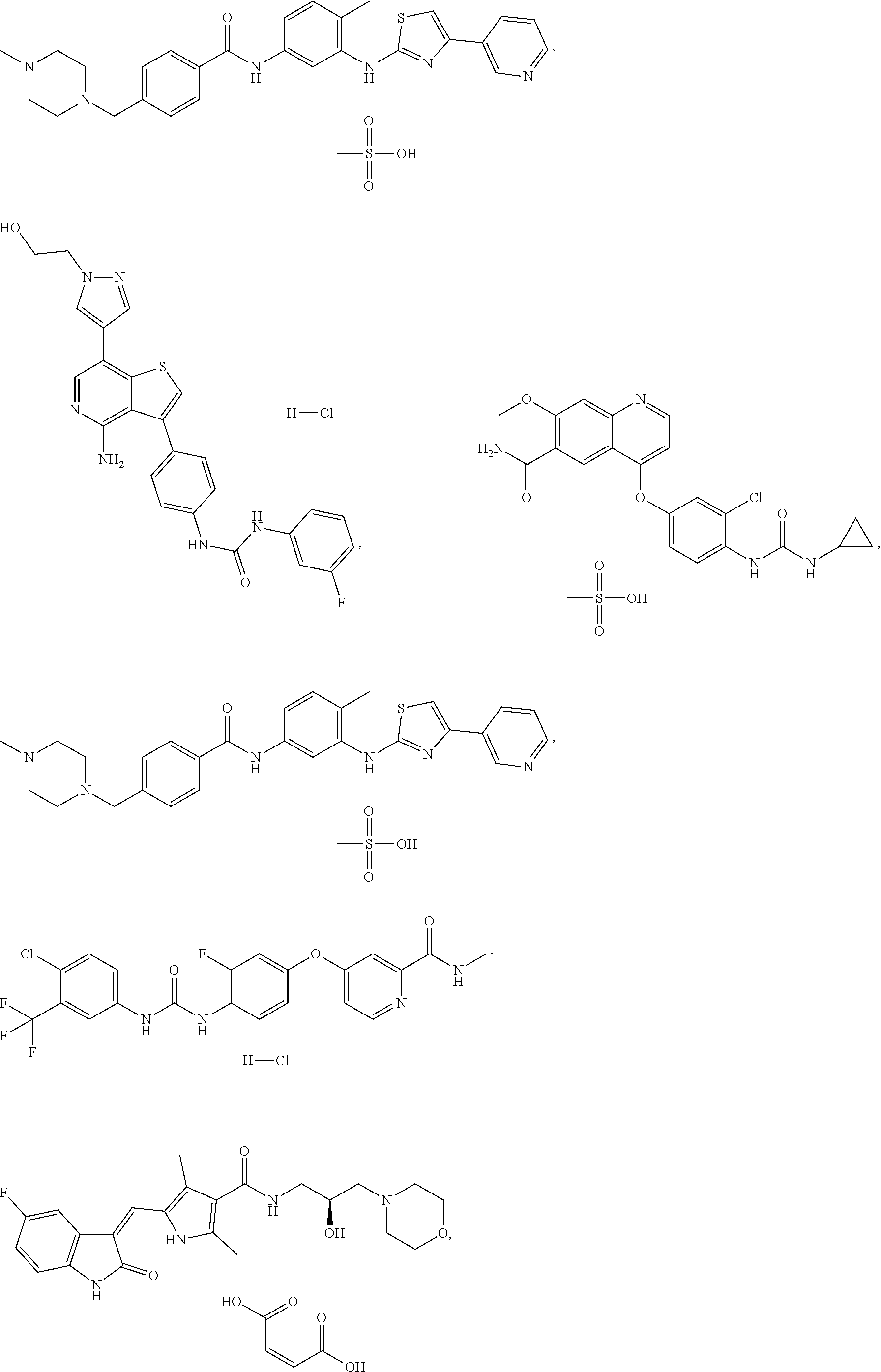

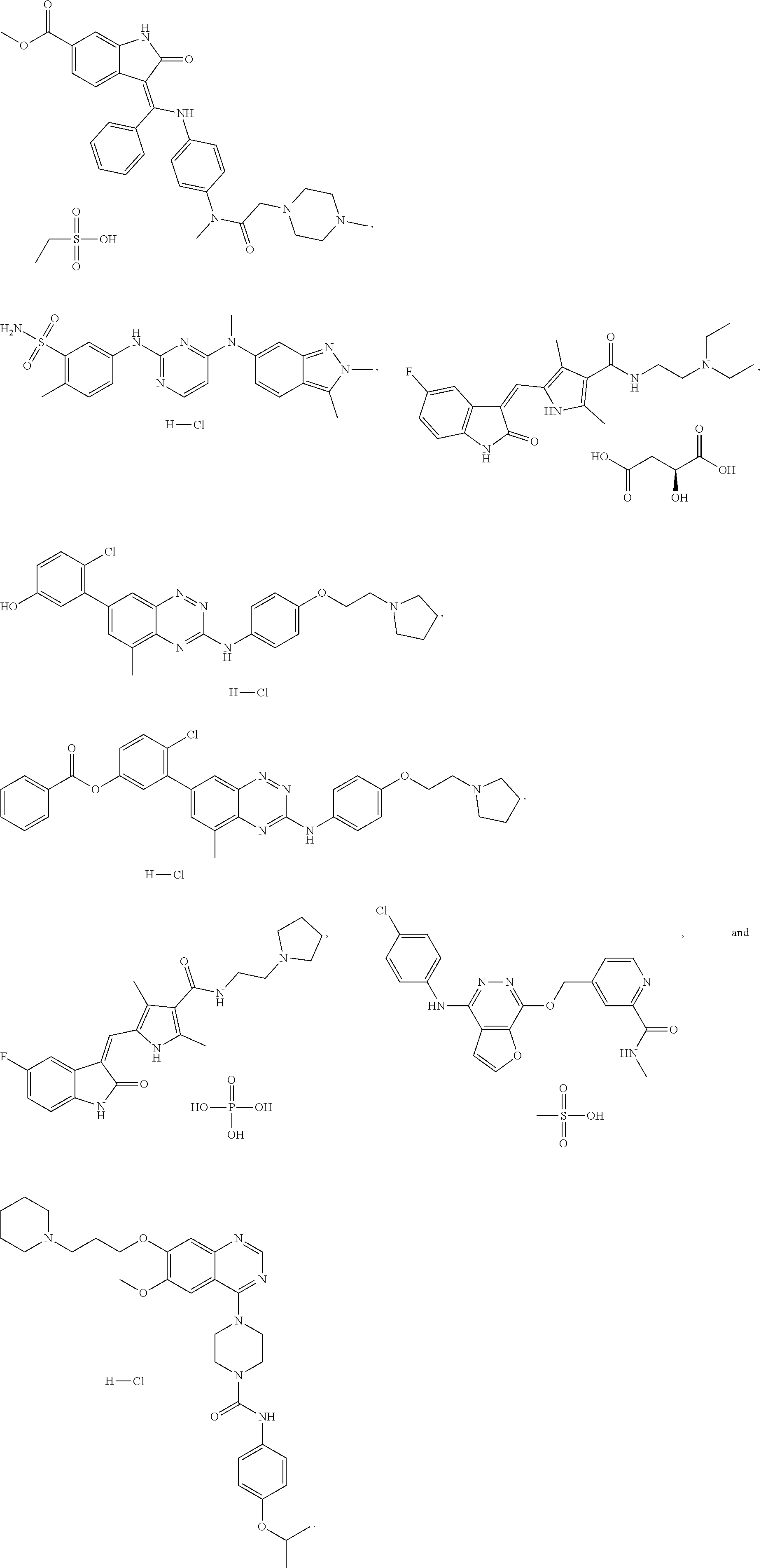

In some embodiments, the disclosure relates to a method of making or enriching NO enteric neurons in a culture by first exposing the cell culture comprising pluripotent stem cells to a series of tissue culture medium. In some embodiments, the tissue culture medium is one of the following:

E8-C, hPSC Medium for Maintenance

Combine Essential 8-Flex supplement (20 μl ml⁻¹) with Essential 8™ Flex Medium. Store at 4° C. (use within 2 weeks).

Cocktail A, first ENC differentiation medium

Combine BMP4 (1 ng ml⁻¹), SB431542 (10 μM), CHIR 99021 (600 nM), with Essential 6™ Medium. Store at 4° C. (use within 2 weeks).

Cocktail B, second ENC differentiation medium

Combine SB431542 (10 μM), CHIR 99021 (1.5 μM), with Essential 6™ medium. Store at 4° C. (use within 2 weeks).

Cocktail C, third ENC differentiation medium

Combine SB431542 (10 μM), CHIR 99021 (1.5 μM), Retinoic Acid (1 μM), with Essential 6™ medium. Store at 4° C. (use within 2 weeks).

NC-C, ENC medium for spheroid maintenance

Combine FGF2 (10 ng ml⁻¹), CHIR 99021 (3 μM), N2 Supplement (10 μl ml⁻¹), B27 Supplement (20 μl ml⁻¹), Glutagro (10 μl ml⁻¹), MEM Nonessential Amino Acids (10 μl ml⁻¹), with Neurobasal® Medium. Store at 4° C. (use within 2 weeks).

EN-C, EN medium for differentiation and maintenance

Combine GDNF (10 ng ml⁻¹), Ascorbic Acid (100 μM), N2 Supplement (10 μl ml^{31 1}), B27 Supplement (20 μl ml⁻¹), Glutagro (10 μl ml⁻¹), MEM Nonessential Amino Acids (10 μl ml⁻¹), with Neurobasal® Medium. Store at 4° C. (use within 2 weeks).

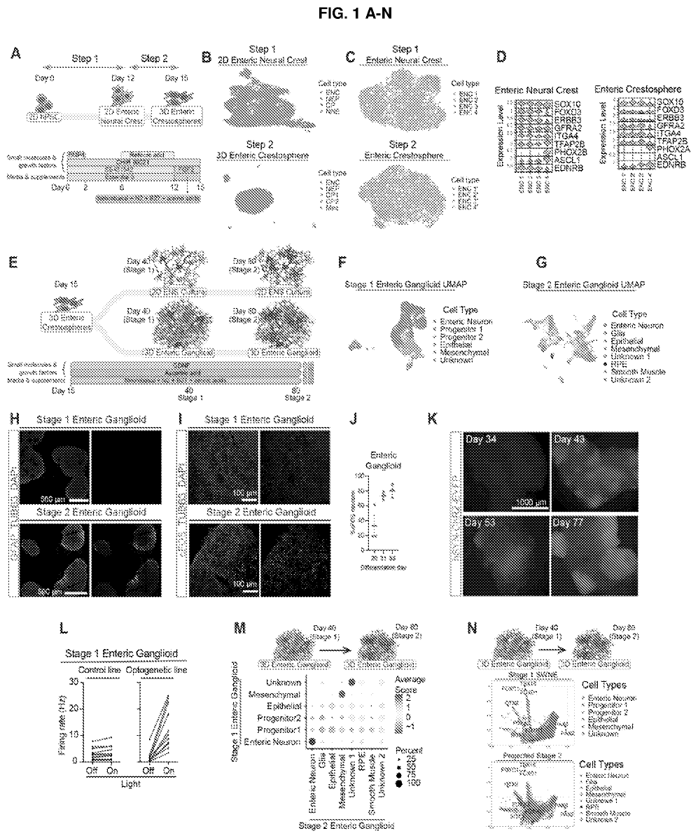

The disclosure relates to a method of culturing any of the compositions disclosed herein with one or more of the cell culture mediums disclosed herein and one or a plurality of PDFR inhibitors. The disclosure relates to a method of differentiating a neural crest cells into a 2 dimensional or three dimensional ganglioid or spheroid comprising exposing one or a plurality of human pluripotent stem cells to GDNF, ascorbic acid, Neurobasal™ (from ThermoFisher), n2 and B27 complement and one or a plurality of PDGFR inhibitors. The disclosure relates to a method of differentiating a neural crest cells into a 2 dimensional or three dimensional ganglioid or spheroid comprising exposing one or a plurality of human pluripotent stem cells to the differentiators factors in FIG. 1E and one or a plurality of PDGFR inhibitors. In some embodiments, the PDGFR inhibitor does not include PP121 or a salt thereof.

The disclosure relates to a method of purifying enteric neurons comprising exposing the neurons in culture from human pluripotent stem cells to one or a plurality of antibodies specific to the biomarkers expressed by the cells. In some embodiments, the biomarker on the enteric neurons are at least 70% sequence identity to at least one or a combination of CD24, CD45RA, CD57, CD63, CD71, CD121b, CD147, CD164, CD184, CD193, CD243, and CD275.

The disclosure also relates to a method of screening for induction of NO or agents that induce NO induction in cells comprising exposing an agent, such as a pharmaceutical compound that is a candidate for a treatment of gut motility disorder, to one or more compositions disclosed herein. The disclosure also relates to a method of screening for toxicity of therapeutic efficacy of an agent comprising exposing the agent, such as a pharmaceutical compound that is a candidate for a treatment of gut motility disorder, to one or more compositions disclosed herein.

Other embodiments are described in the following non-limiting Examples. Various publications, including patents, published applications, technical articles and scholarly articles are cited throughout the specification. Each of these cited publications is incorporated by reference herein in its entirety. The publications include the co-pending provisional application filed on Jan. 3, 2021, and entitled “Methods of Treating Gut Motility Disorders”, as U.S. Ser. No. 63/296,151, which is herein incorporated by reference in its entirety. The publications also include the co-pending PCT application PCT Serial Number PCT/US19/68447, which is herein incorporated by reference in its entirety.

### EXAMPLE

### Example 1. hPSC-Derived Enteric Ganglioids Model Human ENS Development and Function

Here, we describe an experimental system for deriving ENS tissue from human pluripotent stem cells (hPSCs) that recapitulate the remarkable cellular diversity of the human ENS. These three-dimensional (3D) cultures, termed enteric ganglioids, along with two-dimensional (2D) ENS cultures provide scalable sources of human enteric neurons and glia, that are compatible with a wide array of high-throughput applications. We use single cell transcriptomics to map cell-type specific molecular features of human enteric neurons and glia which offers new strategies for their enrichment, isolation, or functional targeting. We leverage hPSC-derived enteric ganglioids as a model system to investigate the development of NO neurons, characterize their molecular and physiological properties and identify clinically relevant strategies to modulate their function in vitro and in the mouse colon ex vivo. Further, we demonstrate the extensive engraftment and regenerative potential of NO neuron ganglioids in the colon of adult mice, providing a new xenograft model to study the human ENS in vivo.

Derivation of Enteric Ganglioids from hPSCs to Model Development, Function and Molecular Diversity of the Human ENS

The ENS is derived from the vagal and sacral neural crest (NC). Vagal NC cells extensively migrate and colonize the entire length of the GI tract, whereas sacral NC cells only colonize the most distal end of the colon (Serbedzija et al., 1991; Burns and Douarin, 1998; Heanue and Pachnis, 2007; Nagy and Goldstein, 2017). We have previously established hPSC differentiation methods to derive enteric neural crest cells (ENCs) under highly defined conditions (FIG. 1A) (Barber et al., 2019; Fattahi et al., 2016). This protocol involves two steps that follow embryonic NC development. In step 1, we induce enteric neural crest by activating bone morphogenic protein (BMP) and Wnt signaling in combination with retinoic acid (RA) treatment. RA caudalizes the differentiating NC, specifying a vagal NC identity. In step 2, we generate enteric crestospheres in the presence of Wnt and fibroblast growth factor (FGF) signaling (Barber et al., 2019; Fattahi et al., 2016).

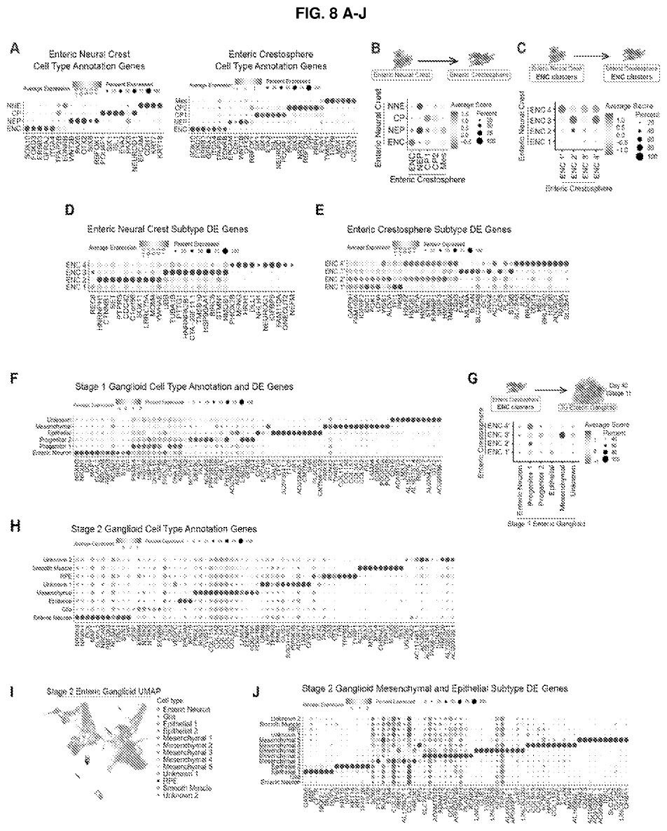

To characterize these developmental processes in the human ENS lineages at the molecular level, we performed single cell RNA-seq (scRNA-seq) on enteric neural crest and enteric crestospheres. During the enteric neural crest stage, four transcriptionally distinct cell types are present: enteric neural crest (ENC) (SOX10⁺, FOXD3⁺), neuro-epithelial progenitor (NEP) (WNT2B⁺, PAX6⁺), cranial placode (CP) (SIX1⁺, EYA2⁺) and non-neural ectoderm (NNE) (EPCAM⁺, CDH1⁺) (FIG. 1B top, FIG. 8A left). In the next step, suspension culture of enteric neural crest cells serves as a purification strategy that leads to enteric crestospheres consisting primarily of ENCs with a small population of NEPs, two CP clusters (CP1 and CP2), and a mesenchymal (Mes) (TWIST1⁺, MSX1⁺) cluster (FIG. 1B bottom, FIG. 8A right). Module scoring the transcriptional signature of cell types in step 1 and step 2 verifies the shared transcriptional identity of the ENC clusters (FIG. 8B).

Further subclustering of the ENC populations identified four subtypes (ENC 1-4 for the enteric neural crest stage and ENC 1′-4′ for the enteric crestospheres) that differentially express canonical ENC markers such as SOX10, EDNRB, TFAP2B and FOXD3, and are chronologically transitioning from PHOX2B to PHOX2A expression (FIGS. 1C and D). To study how ENCs progress during the differentiation steps, we module scored ENC 1-4 transcriptional signatures in the enteric crestosphere ENC 1′-4′ (FIG. 8C). ENC 1′ and 2′ showed high transcriptional similarity to ENC 4, and ENC 2 and 3, respectively, the three most transcriptionally distinct ENC subtypes (FIG. 8C, FIGS. 8D and E).

hPSC-derived ENCs are previously shown to recapitulate key migratory features of ENS precursors in health and disease and are capable of giving rise to enteric neurons upon further differentiation (Barber et al., 2019; Fattahi et al., 2016) but their ability to generate the diverse array of neuronal and glial subtypes that comprise the human ENS has not been characterized. To determine the potential of enteric crestospheres to differentiate into ENS cell types, we established 2D and 3D culture conditions that facilitate the transition of ENCs into mature ENS cell types (FIG. 1E). While 2D culture offers unique technical advantages for applications such as high content imaging assays, we chose to focus primarily on 3D cultures, termed enteric ganglioids, given their scalability and potential for capturing higher order cell-cell interactions that occur in the developing and adult ENS tissue. In addition, 3D culture platforms are technically advantageous in applications such as cell therapy.

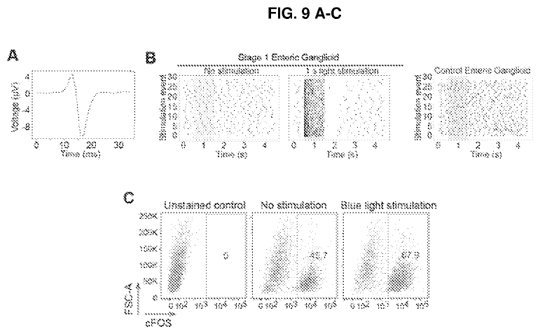

To define the cellular composition of enteric ganglioids we performed single nuclei RNA-seq (snRNA-seq) on stage 1 (differentiation day 35-50) and stage 2 (differentiation day 70-90) enteric ganglioids (FIG. 1E). Unbiased clustering of stage 1 enteric ganglioids revealed a large population of enteric neurons, two progenitor populations and small populations of contaminating epithelial cells, mesenchymal cells, and one cluster of unknown identity (FIG. 1F, FIG. 8F). Module scoring based lineage analysis revealed that the progenitor 1 population shared high transcriptional similarity to ENC 2′ and 4′. Furthermore, the mesenchymal population was highly similar to ENC 3′ (FIG. 8G). Importantly, stage 2 enteric ganglioids contained enteric glia in addition to enteric neurons, indicating that gliogenesis follows neurogenesis during in vitro differentiation, which is consistent with the in vivo developmental timeline (FIG. 1G) (Rothman et al., 1986; Young et al., 2003). We confirmed the presence of glia in our stage 2 enteric ganglioids by immunostaining for GFAP (FIG. 1H). Stage 2 ganglioids contained a larger proportion of contaminating epithelial cells, mesenchymal cells, and two unknown clusters (FIG. 1G, FIG. 8H). At stage 2 epithelial and mesenchymal populations showed higher transcriptional diversity compared to stage 1 clusters and could be further sub-clustered into two unique epithelial populations and five unique mesenchymal populations (FIGS. 8I and J). Additionally, at this later stage, contaminating retinal pigmented epithelium (RPE) and smooth muscle cell populations emerged (FIG. 1G, FIG. 8H). To evaluate the functional maturation state of the enteric neurons over the course of differentiation, we evaluated the expression of the neuronal activity marker cFOS. Neuronal depolarization leads to expression of cFOS, a proto-oncogene that has been used as a marker for neuronal activity (Hunt et al., 1987; Bullitt, 1990; Santos et al., 2018). cFos expression increased as the enteric ganglioids progressed during differentiation (FIGS. 1I and J). To demonstrate synaptic maturation and electrical excitability of enteric neurons within ganglioids, we used optogenetics by differentiating a reporter hESC line that expresses enhanced yellow fluorescent protein (EYFP) tagged channelrhodopsin-2 under control of the human synapsin promoter (Steinbeck et al., 2016). EYFP was readily detectable as early as day 43 (FIG. 1K). Light stimulation of stage 1 ganglioids increased electrical firing rates, as detected by microelectrode array (MEA) (FIG. 1L, FIGS. 9A and B), leading to increased cFOS expression as compared to unstimulated enteric ganglioids (FIG. 9C). Thus, stage 1 and stage 2 enteric neurons are functional and continue to gain maturity over time.

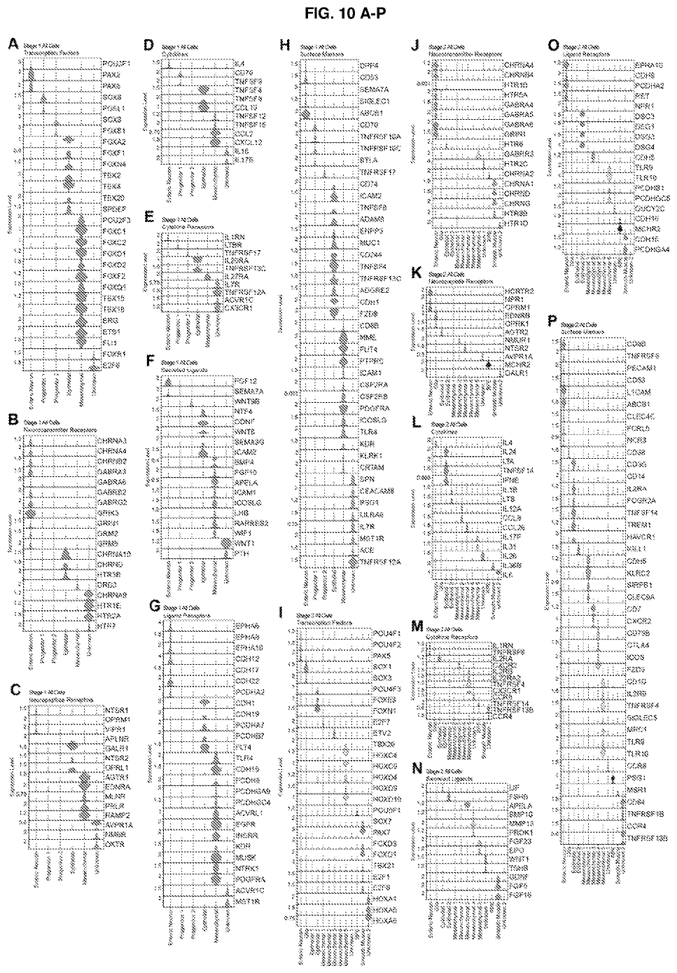

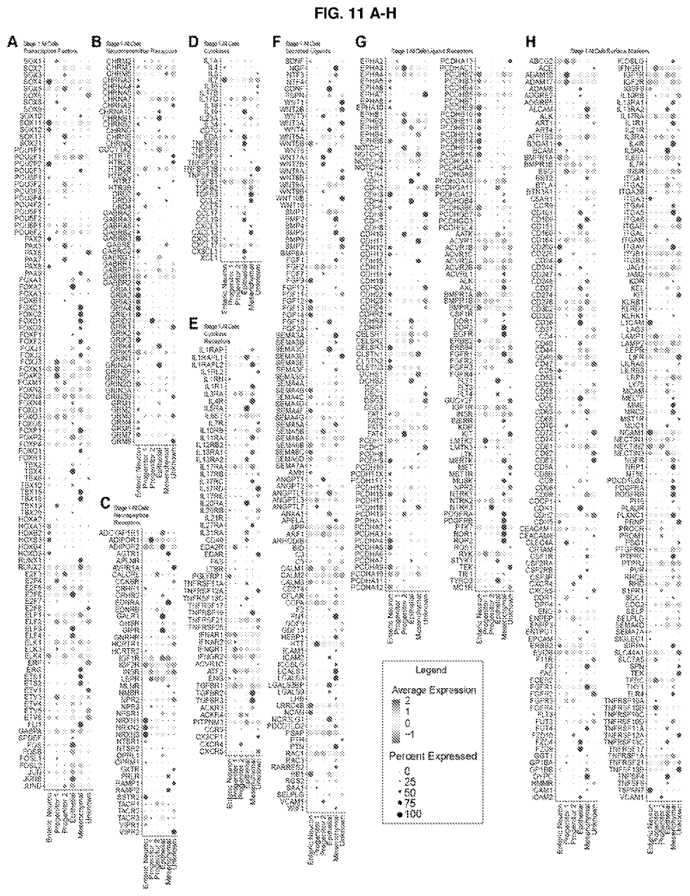

Next, we explored the transcriptional differences, lineage relationships, and functional properties of stage 1 and stage 2 enteric ganglioids. Many genes, including transcription factors, neurotransmitter receptors, neuropeptide receptors, cytokines and their receptors, secreted signaling ligands and their receptors, and surface markers were exclusively expressed (detected in >25% of cells in a single cluster) by each population in the stage 1 and stage 2 enteric ganglioids (FIG. 10A-P). Other genes in these categories, while not exclusively expressed, showed differential expression between cell types (FIG. 11 A-H, FIG. S5A-H). FIG. S5 data not shown, but it describes an expression profiles of selected gene categories in stage 2 enteric ganglioid cell types. The data of S5 were disclosed in Majd et al., “hPSC-Derived Enteric Ganglioids Model Human ENS Development and Function”, bioRxiv, posted Jan. 3, 2022, which is incorporated by reference in its entirety. To determine the lineages shared between stage 1 and 2 enteric ganglioids, we conducted module scoring based lineage analysis. Transcriptional signatures were highly conserved between stage 1 and 2 enteric neurons and mesenchymal cells (FIG. 1M). Interestingly, the glia population was most transcriptionally similar to the two progenitor populations from stage 1 (FIG. 1M). We confirmed these transcriptional similarities between stage 1 and stage 2 enteric ganglioid cell types by generating similarity weighted non-negative embeddings (Wu et al., 2018) (SWNE) by projecting stage 2 ganglioid cells onto the stage 1 SWNE (FIG. 1N). Stage 2 cell types mapped to similar SWNE space positions as the matched stage 1 cell types, suggesting similar expression patterns and lineage continuation (FIG. 1N).

To compare the cellular diversity between our 2D ENS cultures and enteric ganglioids we performed scRNA-seq on 2D cultures in stage 2. Similar to the 3D ganglioids, clustering and annotation by expression of key marker genes revealed enteric neurons, glia, epithelial and mesenchymal cells, and two unknown populations (FIGS. S6 A and B). Figure S6 data not shown, but it describes comparison of stage 2 enteric ganglioid and 2D ENS cultures. The data of S6 were disclosed in Majd et al., “hPSC-Derived Enteric Ganglioids Model Human ENS Development and Function”, bioRxiv, posted Jan. 3, 2022, which is incorporated by reference in its entirety. Projection of 2D cells into 3D ganglioid SWNE space showed conserved expression patterns between 2D and 3D enteric neurons, glia, mesenchymal cells and unknown cluster 1 (FIG. S6C). These observations were confirmed by calculating the Spearman correlation of the expression of 3000 shared variably expressed genes (Seurat anchor features) between stage 2 ganglioid and 2D ENS culture cell types (FIG. S6D). Importantly, 2D and 3D enteric neurons and glia showed high correlations of 0.83 and 0.76, respectively (FIG. S6D). Additionally, the mesenchymal and unknown 1 clusters showed a high correlations (0.82, and 0.80, respectively), while the epithelial clusters showed a modest correlation of 0.29 (FIG. S6D). Interestingly, the 2D specific unknown cluster 2 showed a moderate correlation to the glia cluster (FIG. S6D). Taken together, these data indicate that the enteric neurons and glia generated by the new 3D differentiation format are highly transcriptionally similar to their 2D counterparts, while the off-target/contaminating cell types may vary in composition and transcriptional identity between the two formats.

hPSC-Derived Enteric Ganglioids Recapitulate the Neuronal Diversity of the Human ENS

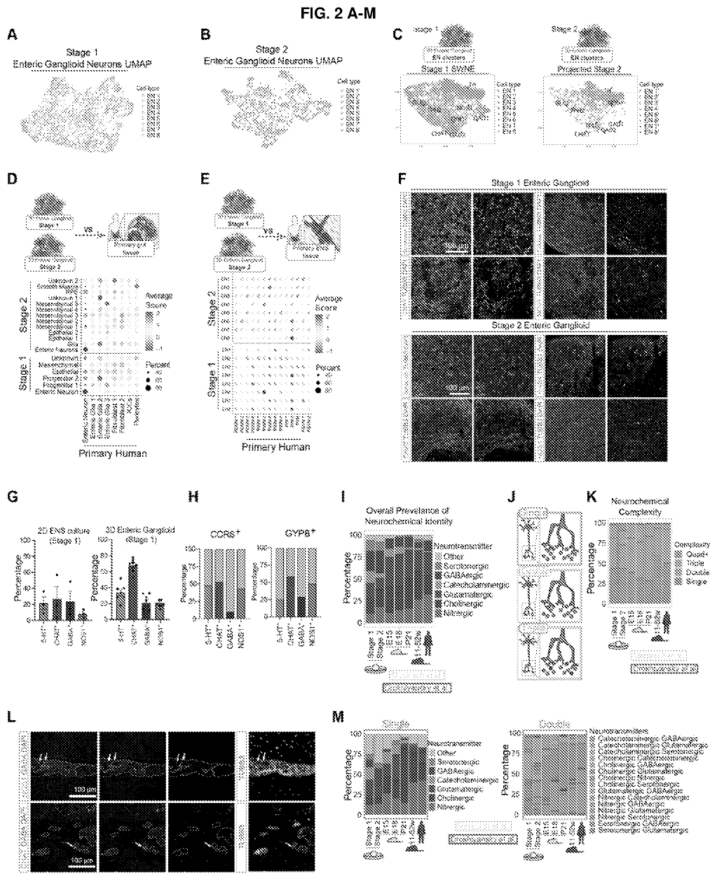

Recent characterization of human and mouse primary enteric neurons at single cell resolution have revealed many transcriptionally distinct clusters of enteric neurons (Drokhlyansky et al., 2020; Morarach et al., 2021). To characterize the diversity of our hPSC-derived enteric neurons, we sub-clustered the neuronal populations in stage 1 and 2 ganglioids. Eight transcriptionally distinct neuronal subtypes (EN 1-8) were identified in the stage 1 enteric neurons (FIG. 2A, FIGS. S7A and B). FIG. S7 data not shown, but it describes Profiling of enteric neuron subtypes present in stage 1 and 2 enteric ganglioids. The data of S7 were disclosed in Majd et al., “hPSC-Derived Enteric Ganglioids Model Human ENS Development and Function”, bioRxiv, posted Jan. 3, 2022, which is incorporated by reference in its entirety. Module scoring revealed that EN 1 and EN 6 showed higher similarities with enteric crestosphere ENCs potentially representing earlier stage neuronal populations (FIG. S7C). For the stage 2 enteric neurons, sub-clustering analysis similarly revealed eight distinct subtypes of enteric neurons (EN 1′-8′) (FIG. 2B, FIGS. S7D and E). Comprehensive analysis of functionally and technically relevant gene categories revealed transcription factors, neuropeptides and their receptors, neurotransmitter receptors, cytokines and their receptors, secreted signaling ligands and their receptors, and surface markers that were exclusively expressed by enteric neuronal subtypes in stage 1 and 2 (FIG. S8A-Q). FIG. S8 data not shown, but it describes Identification of cluster specific markers by gene category in stage 1 and 2 enteric ganglioid neuronal subtypes. The data of S8 were disclosed in Majd et al., “hPSC-Derived Enteric Ganglioids Model Human ENS Development and Function”, bioRxiv, posted Jan. 3, 2022, which is incorporated by reference in its entirety. Many genes in these categories, while not exclusively expressed, showed differential expression between enteric neuron subtypes (FIG. S9A-J, FIG. S10A-J). FIG. S9 and S10 data not shown, but S9 describes Expression profiles of selected gene categories in stage 1 enteric ganglioid neuronal subtypes and S10 describes Expression profiles of selected gene categories in stage 2 enteric ganglioid neuronal subtypes. The data of S9 and S10 were disclosed in Majd et al., “hPSC-Derived Enteric Ganglioids Model Human ENS Development and Function”, bioRxiv, posted Jan. 3, 2022, which is incorporated by reference in its entirety. This signifies the remarkable functional diversity of cell types in the differentiated ganglioids.

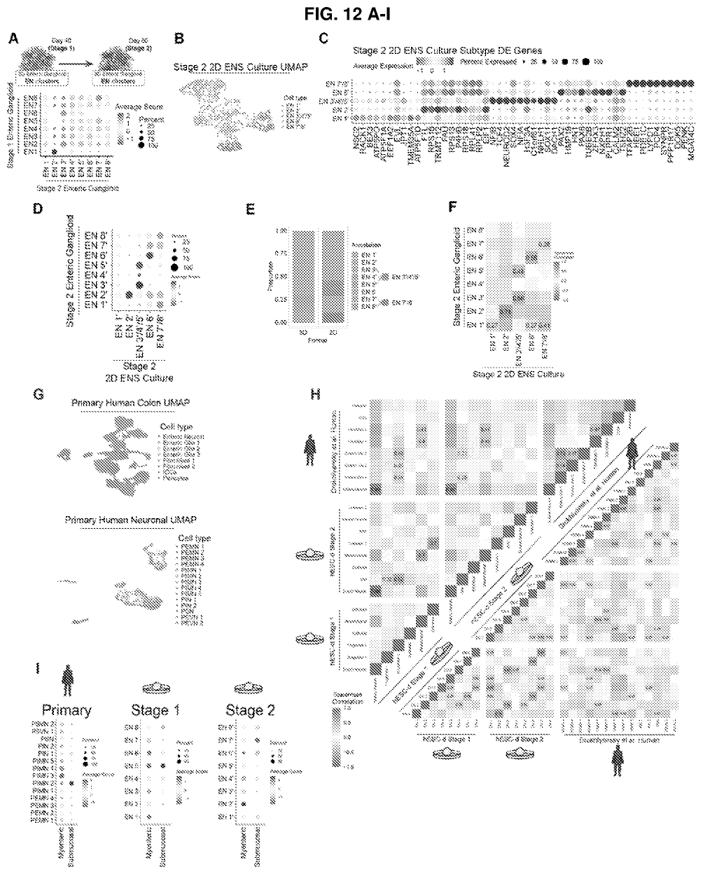

Next, we determined the lineage similarities between the stage 1 and 2 enteric neuron subtypes. Module scoring revealed the highest transcriptional similarity between EN 1 and EN 2′ (FIG. 12A). Other stage 2 enteric neuron subtypes shared modest transcriptional similarities with multiple stage 1 enteric neuron subtypes (FIG. 12A). To confirm this observation, we projected stage 2 enteric neurons into stage 1 enteric neuron SWNE space. In agreement, many stage 1 and 2 enteric neurons showed similar expression patterns based on regional overlap of EN 1 and EN2′, EN 2 and 6 with EN 3′, EN 4 and 8 with EN 1′ and 7′, and EN 7 with EN 6′ in the SWNE space (FIG. 2C). Interestingly, very few stage 2 enteric neurons overlapped with stage 1 EN 3 and 5, suggesting these may be transient neuronal subtypes (FIG. 2C).

To compare the neuronal diversity between the 2D cultures and ganglioids, we performed parallel sub-clustering analysis of the 2D stage 2 enteric neurons (FIG. 12B). 2D enteric neurons clustered into five distinct enteric neuron populations (FIGS. 12B and C). Module scoring revealed that all ganglioid neuronal signatures are present in the 2D enteric neurons, however, EN 3′-5′ and EN 7′ and 8′ clustered together in the 2D enteric neuron dataset (FIGS. 12D and E). These results were further supported by Spearman correlation analysis of 3000 anchor features shared between 2D and ganglioid enteric neurons that revealed positive correlations between 2D EN 3′-5′ and ganglioid EN3′ and EN5′, as well as 2D EN 7′/8′ and ganglioid EN 7′ (FIG. 12F).

To validate that hPSC-derived enteric ganglioids recapitulate in vivo ENS biology, we compared our stage 1 and 2 ganglioids to a snRNA-seq dataset of the primary human colon previously published by Aviv Regev and Colleagues (Drokhlyansky et al., 2020) (FIG. 12G). Remarkably, module scoring of ganglioid cell type signatures on relevant primary cell types demonstrated that in vitro and in vivo enteric neurons were highly similar (FIG. 2D). Likewise, correlating the expression of 3000 anchor features shared between the three datasets showed Spearman correlation values of 0.89 and 0.87 between the primary and the stage 1 and 2 enteric neurons, respectively (FIG. 12H top). Further, module scoring and Spearman correlation analyses revealed that stage 1 and 2 enteric neuron subtypes represent transcriptional signatures of primary neuron subtypes from all neuron classes (FIG. 2E, FIG. 12H bottom). These data demonstrate that our ganglioids capture the diversity of neuronal transcriptional identities in the human ENS.

Another important component of an enteric neuron's identity is its location in the myenteric or submucosal plexus. In order to generate myenteric and submucosal gene signatures, we utilized metadata associated with the human samples sequenced by Drokhlyansky et al. denoting the tissue layer from which each sample was collected. Module scoring the primary enteric neuron subtypes with these plexus gene modules found the tissue layer signatures to be mutually exclusive, with each neuronal subtype having a positive score for either one module or the other (FIG. 12I left). Interestingly, this analysis suggests that PEMN, PIMN, PSN and PSVN categories contain both myenteric and submucosal subtypes. However, both PIN subtypes scored positively for the submucosal signatures (FIG. 12I left). Module scoring our stage 1 and 2 enteric neurons suggests that our ganglioids generate both myenteric and submucosal neurons largely recapitulate the mutual exclusivity of these signatures (FIG. 12I middle and right).

Enteric neuron identity is often described based on their neurochemical properties including nitrergic, cholinergic, glutamatergic, catecholaminergic, GABAergic or serotonergic. We first verified the presence of neurons with various neurochemical features in our stage 1 and stage 2 ganglioids by immunostaining (FIG. 2F). These neurochemical markers were consistently represented in both 2D and ganglioid culture formats (FIG. 2G). Intriguingly, our transcriptional analysis of both stages showed that multiple enteric neuron subtypes express the same neurotransmitter markers (FIG. S9B, FIG. S10B). FIG. S9 data not shown, but it describes expression profiles of selected gene categories in stage 1 enteric ganglioid neuronal subtypes. The data of S9 were disclosed in Majd et al., “hPSC-Derived Enteric Ganglioids Model Human ENS Development and Function”, bioRxiv, posted Jan. 3, 2022, which is incorporated by reference in its entirety. For example, neurons in EN 4, 5 and 8 neurons expressed NOS1 which is the enzyme that produces NO and is a marker for nitrergic neurons. Additionally, individual enteric neuron subtypes expressed markers for multiple neurotransmitters. For example, EN 5 expressed NOS1 and GAD1 which is a gabaergic marker and EN 8 expressed the cholinergic marker SLC5A7 and the glutamatergic marker SLC17A6 (FIG. S9B). We next performed flow cytometry to validate the co-expression of neurotransmitter markers within neuronal subclusters. As a proof of principle, we used two surface markers, CCR6 and GYPB, which specifically expressed in stage 1 EN 8 (FIG. S9J), to label this subcluster and quantify the proportion of serotonin⁺, CHAT⁺, GABA⁺, and NOS1⁺ enteric neurons. All four neurotransmitter markers were detected in CCR6⁺ and GYPB⁺ populations confirming that transcriptionally distinct enteric neuron subclusters are not defined by a single neurotransmitter identity (FIG. 2H). This suggests that a single neurotransmitter cannot serve as a specific marker for annotation of transcriptionally distinct enteric neuron subtypes, and reaffirms the hypothesis that each neuron can take on multiple neurochemical identities.

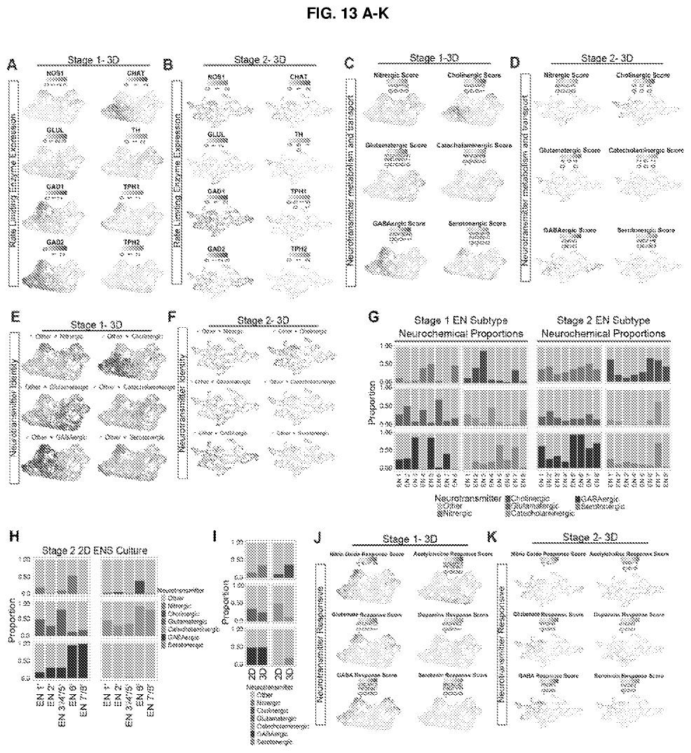

To further explore this hypothesis at the single cell resolution, we designed a stringent two-step approach to define an enteric neuron's neurochemical identity. In the first step, we identified neurons that expressed the hallmark rate limiting neurotransmitter synthesis enzymes (FIGS. 13A and B). In the second step, we module scored these neurons based of their expression of a curated list of neurotransmitter metabolism enzymes and transport proteins (Table S1, FIGS. 13C and D). Neurons that passed both steps were binned into a particular class of neurotransmitter identity (FIGS. 13E and F). For example, neurons were annotated as nitrergic if they expressed NOS1 and scored highly for NO metabolism and transport genes NOS1AP, ARG1/2, ASL and ASS1. We found that all EN subtypes, both in 2D and 3D, contained neurons from every neurotransmitter identity class (FIG. 13G-I). Furthermore, at the single cell level, many neurons were equipped to synthesize multiple neurotransmitters (FIG. 13A-F).

TABLE S1

| Cholinergic | Nitrergic | Dopamanergic | Serotonergic | GABAergic | Glutamatergic |
|---|---|---|---|---|---|
| CHAT | NOS1 | TH | TPH1 | GAD1 | GLUL |
| SLC18A3 | NOS1AP | PAH | TPH2 | GAD2 | SLC17A6 |
| ACHE | ARG1 | DDC | DDC | SLC32A1 | SLC17A7 |
| SLC5A7 | ARG2 | TYR | TDO2 | | SLC17A8 |
| | ASL | SPR | IDO1 | | |
| | ASS1 | QDPR | IDO2 | | |
| | | TAT | SLC6A4 | | |
| | | SLC6A3 | SLC18A2 | | |
| | | QDPR | | | |
| | | SLC18A2 | | | |

A defining aspect of an enteric neuron's function is its ability to sense and respond to specific neurotransmitters released by other neurons. As a first step towards mapping the neuronal communication networks in enteric ganglioids, we profiled the expression of neurotransmitter receptor gene families in the EN subtypes. Interestingly, neurons in stage 1 and 2 showed the same phenomenon of falling into one of three major neurotransmitter responsive groups: NO/serotonin/GABA/glutamate responsive, acetylcholine responsive, or dopamine responsive (FIGS. 13J and K). For example, at stage 1, EN 3-5, 7 and 8 neurons are predicted to be responsive to NO, serotonin, GABA and glutamate, EN 2 and 6 neurons are predicted to be responsive to acetylcholine and a subset of EN 1 neurons are predicted to be responsive to dopamine (FIG. 2A, FIG. 13J). It is important to note that many individual neurotransmitter receptor genes within the same family were differentially expressed between our enteric neuron subtypes (FIG. S9C, FIG. S10C). For example, within the family of acetylcholine receptors expressed by stage 1 enteric neuron subtypes, CHRM1 and CHRNA10 are exclusively expressed by EN 6, while CHRNB3 is exclusively expressed by EN 5 (FIG. S9C). These observations, in conjunction with the multiple neurotransmitter synthesis properties present in individual enteric neurons, highlights the complexity of ENS circuits, where a single neuron may both synthesize and respond to multiple neurotransmitters. Further, neurons can show subtype-specific responses to neurotransmitters depending on the receptor family member expression.

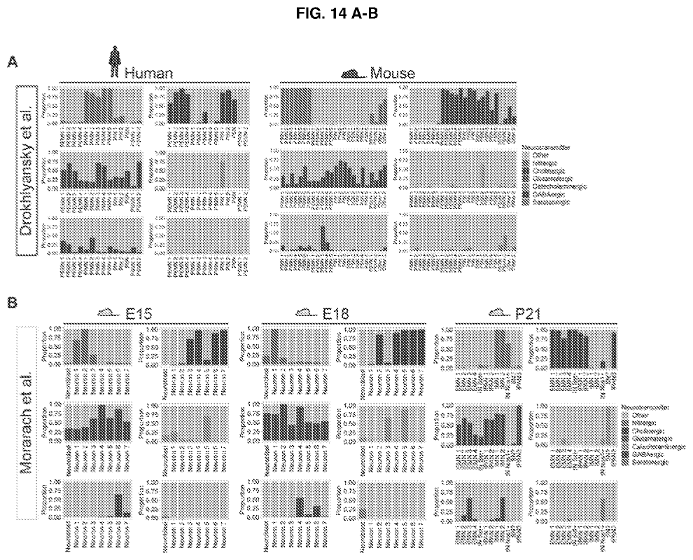

To verify that these complex neurochemical and transcriptional identities are physiologically relevant, we applied the same characterization criteria to both primary mouse and human ENS datasets (Drokhlyansky et al., 2020; Morarach et al., 2021) (FIGS. 14A and B). Interestingly, previously annotated subtypes are predicted to contain enteric neurons that synthesize multiple neurotransmitters, confirming our in vitro observations (FIGS. 14A and B). We then compared the overall abundance of neurons within each neurochemical class across each dataset irrespective of whether a neuron is predicted to synthesize multiple neurotransmitters (FIG. 2I). We found that our ganglioids recapitulate temporal features of ENS development and maturation, such as the increase in NO neurons and loss of catecholaminergic neurons over time (Baetge and Gershon, 1989; Baetge et al., 1990; Bergner et al., 2014; Lake and Heuckeroth, 2013; Obermayr et al., 2013) (FIG. 2I). Additionally, by concatenating the individually predicted neurochemical identities, we found that primary enteric neurons also contain complex neurochemical identities, where neurons are predicted to synthesize either one, two, three or more neurotransmitters (FIGS. 2J and K). We confirmed this by immunostaining of primary human colon and identifying neurons that were positive for both GABA and NOS1 or GABA and CHAT (FIG. 2L). Interestingly, hPCS-derived enteric ganglioid neurons show relatively similar proportions of neurotransmitter complexity categories to primary neurons, and the neurochemical complexity appears to change during development in mice (FIG. 2K). The breakdown of neurons belonging to each single and double neurochemical class confirms the presence of similar types of neurons across all datasets (FIG. 2M).

hPSC-Derived Enteric Ganglioids Recapitulate the Glial Diversity of the Human ENS

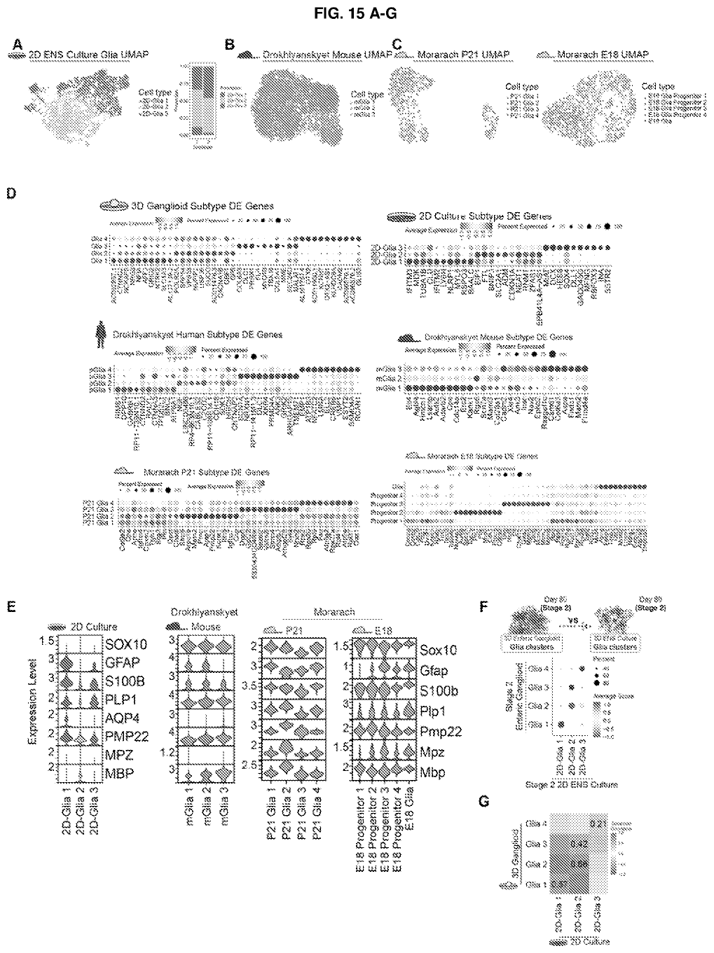

Enteric glia play crucial roles in ENS physiology and disease but their molecular and functional characteristics have remained elusive. We showed that our stage 2 enteric ganglioids and 2D ENS cultures contain glia (FIGS. 1G and H, FIG. S6A). To characterize these hPSC-derived enteric glia and determine if they recapitulate the transcriptional properties of primary enteric glia (Drokhlyansky et al., 2020; Morarach et al., 2021), we sub-clustered the glial population (FIG. 3A) in enteric ganglioids and 2D ENS datasets and performed independent sub-clustering analyses of the primary glia sequenced by Drokhlyansky et al. and Morarach et al. We identified four glial subtypes (Glia 1-4) in the enteric ganglioids in two independent replicates (FIG. 3B). Similarly, clustering of the primary glia in the Drokhlyansky et. al. human dataset showed four distinct subtypes (pGlia 14), differing from the 6 subtypes (three shared and three patient-specific subtypes) originally annotated by the authors (FIG. 3C). Interestingly, visualizing the proportion of glial subtypes isolated from each patient sample in the primary human dataset suggests that the representation of glial subtypes varies from patient to patient possibly due to differences in sample collection (FIG. 3D). Further, we subclustered the transcriptionally distinct glial lineages in our 2D ENS cultures and previously published mouse datasets (FIG. 15A-D). All glial subtypes in 2D ENS cultures, enteric ganglioids and primary datasets expressed canonical glial markers (FIG. 3E, FIG. 15E). We confirmed this using immunofluorescence staining of S100 and GFAP in enteric ganglioids and primary human colon tissue (FIG. 3F). Intriguingly, GFAP transcript was not detected in all glial populations and was restricted to Glia 1 in enteric ganglioids, Glia 1 and Glia 4 in 2D ENS cultures, and was low in human primary glial populations (FIG. 3E, FIG. 15E). Immunofluorescence staining of S100 and GFAP confirmed that these markers are not co-expressed in all glial cells (FIG. 3G). To determine similarities between glial subtypes between the 3D and 2D subclusters, we next compared their transcriptional signatures. Module scoring and Spearman correlation revealed that a single 2D subtype shared the Glia 2 and 3 signatures in 3D enteric ganglioids (FIGS. 15F and G), confirming that all glial subtypes in enteric ganglioids are present in the 2D ENS cultures (FIG. 15F). Module scoring showed that pGlia 1 is the most transcriptionally similar to Glia 1 and 4 subtypes, whereas pGlia 4 is most similar to the Glia 2 and 3 subtypes (FIG. 3H). These data demonstrate that our 2D ENS cultures and enteric ganglioids capture the glial diversity of the human ENS.

Remarkably, we detected high level expression of myelinating markers PMP22, MPZ and MBP in our cultures (FIG. 3E). Similarly, MBP transcript was present in all four human primary enteric glial subtypes, and pGlia3 showed MPZ expression (FIG. 3E). Immunofluorescence staining confirmed the expression of myelin markers in stage 2 ganglioids and human primary colon tissue (FIG. 3I). This is intriguing given the longstanding assumption that myelination does not occur in the ENS

While S100B and PLP1 were expressed by all pGlia subtypes, they were only detected in Glia 1 and Glia 4 (FIG. 3E). On the other hand, MPZ and MBP were predominantly expressed by the other two subtypes Glia 2 and 3 (FIG. 3E). The mutually exclusive expression pattern of some of the canonical glial markers in our enteric ganglioids prompted us to explore their developmental origin. We aimed to infer lineage relationships between our stage 1 progenitor populations and the Glia subtypes. We found that Glia 2 and 3 shared a similar signature with progenitor 1, whereas Glia 1 and 4 were most similar to the progenitor 2 population (FIG. 3J). This may suggest that unique enteric progenitor populations give rise to distinct MPZ⁺/MBP⁺ and GFAP⁺/AQP4⁺/S100B⁺/PLP1⁺ enteric glia (FIG. 3E).

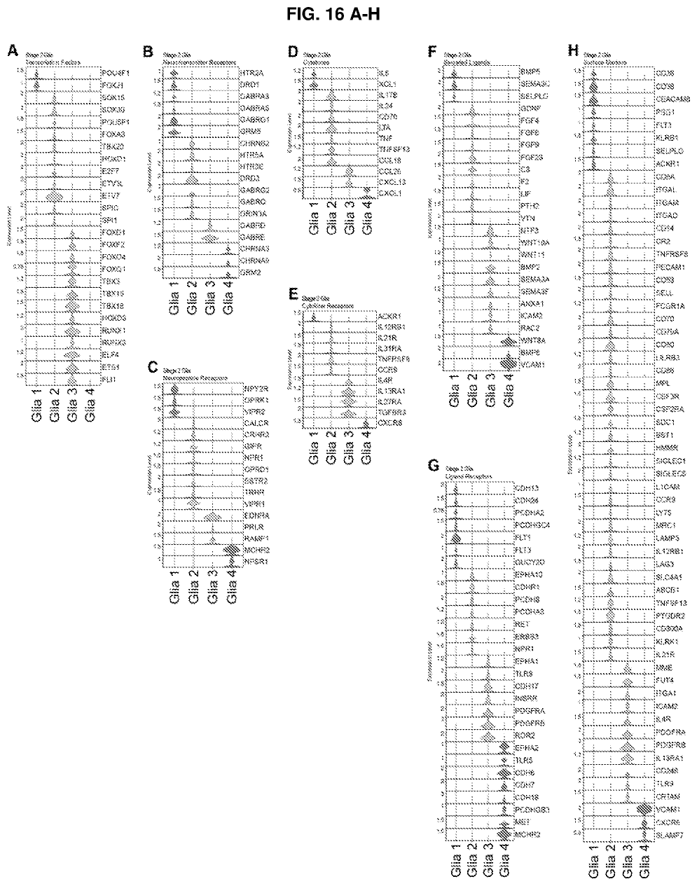

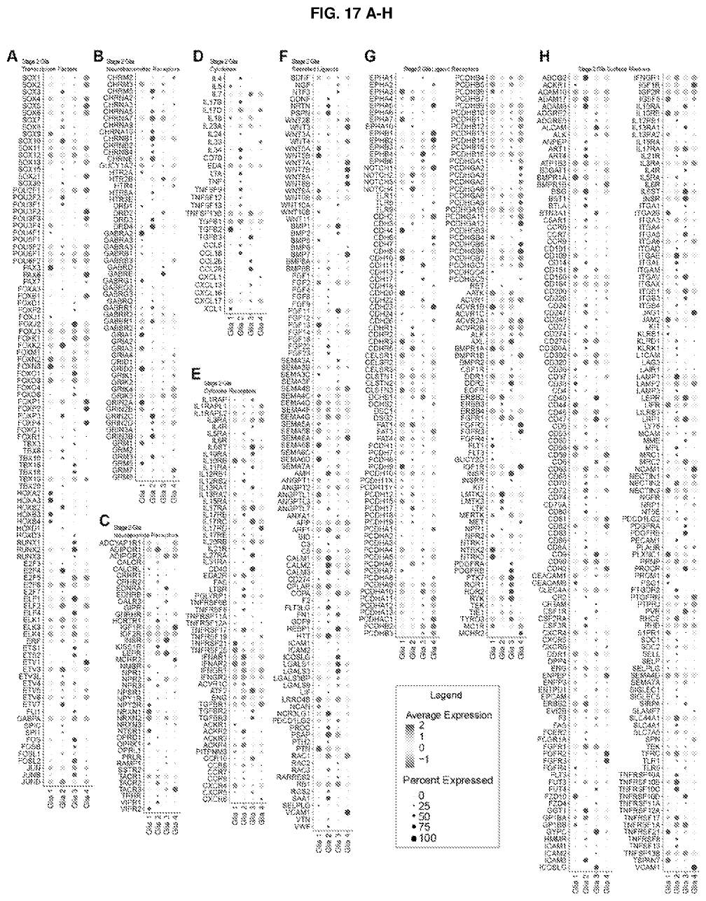

Given that enteric glial diversity has not been comprehensively and transcriptionally profiled, we performed deeper characterization of our stage 2 enteric ganglioid glial populations. Many transcription factors, neurotransmitter receptors, neuropeptide receptors, cytokines and their receptors, secreted signaling ligands and their receptors, and surface markers were exclusively expressed by each glial subtype (FIG. 16 A-H), whereas other genes in these categories are differentially expressed between subtypes (FIG. 17 A-H).

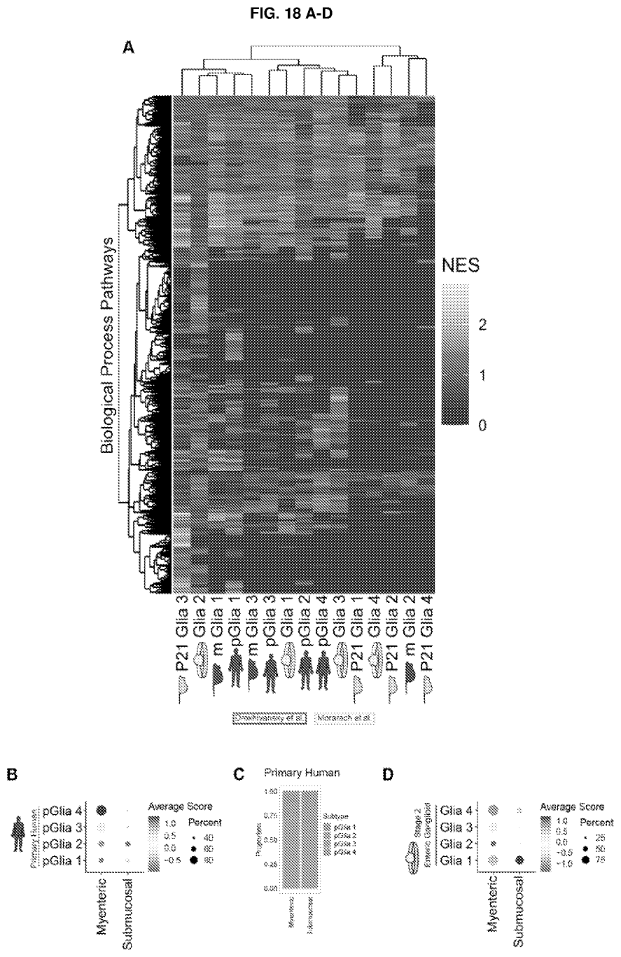

Further, we developed an approach to compare functional features of the glial subtypes in our enteric ganglioids with primary glial subtypes. We performed gene set enrichment analysis (GSEA) using the biological function gene ontology (GO) gene sets on the significantly upregulated gene lists of each mature glia subtype. Next we performed hierarchical clustering of the glial subtypes across all datasets based on the normalized enrichment score of all enriched GO terms present in at least one glial subtype. This analysis revealed three overarching classes of enteric glia conserved between mouse and human (FIG. 18A). Inspection of the GO terms enriched in all glia of a particular class revealed diverse predicted functions of each class (FIG. 3K). Class 1 glia are enriched for terms related to synapse regulation and ion transport while class 2 glia display terms related to adhesion and immune function. Both class 1 and 2 glia also contain terms related to epithelial and endothelial regulation. Interestingly, class 3 glia also show terms related to synapse regulation but uniquely contain terms specific to sensory processes. We next generated myenteric and submucosal glial signatures based on the differentially expressed genes of all primary glia isolated from each plexus. We used these signatures to predict plexus identities for each glial subtype in the human datasets. Similar to the neurons, scoring of the primary human glial subtypes showed mutual exclusion of the tissue layer signature, with pGlia 1 and 2 scoring positively for submucosal and pGlia 3 and 4 scoring positively for myenteric (FIG. 18B). Interestingly, pGlia 4 is predominantly located in the myenteric signature (FIG. 18C). Similar to the neurons, scoring Glia 1-4 with the plexus signatures suggests that our cultures generate glia from both the myenteric and submucosal layers (FIG. 18D). All together, these data indicate that our hPSC-derived enteric glia recapitulate the transcriptional, functional, and regional features of the primary human enteric glia.

### Enteric Ganglioids Enable Comprehensive Characterization of Human NO Neurons

GI motility is directly controlled by the enteric excitatory and inhibitory motor neurons. A large subset of inhibitory neurons use NOS1 to synthesize the neurotransmitter NO that induces relaxation in the smooth muscle tissue (Bredt et al., 1990; Bult et al., 1990; Ward et al., 1992; Young et al., 1992). NO is also an important regulator of mucosal integrity and barrier function. Enteric NO neurons are particularly important due to their involvement in a broad range of motility disorders. Selective loss and dysfunction of NO neurons have been associated with muscular hypercontractility underlying many dysmotility conditions such as achalasia, gastroparesis, intestinal pseudo-obstruction and colonic inertia (Bódi et al., 2019; Rivera et al., 2011).

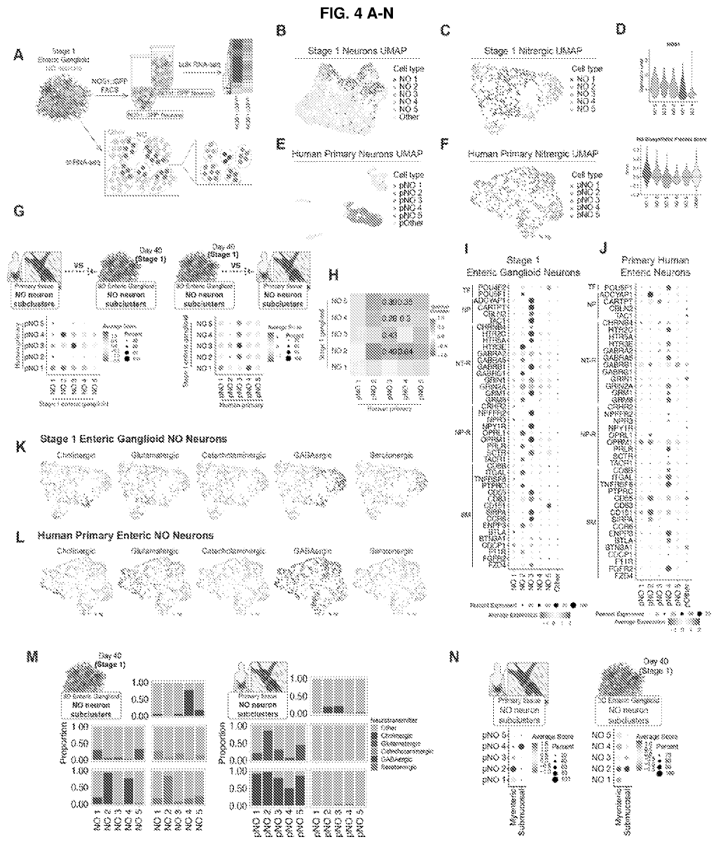

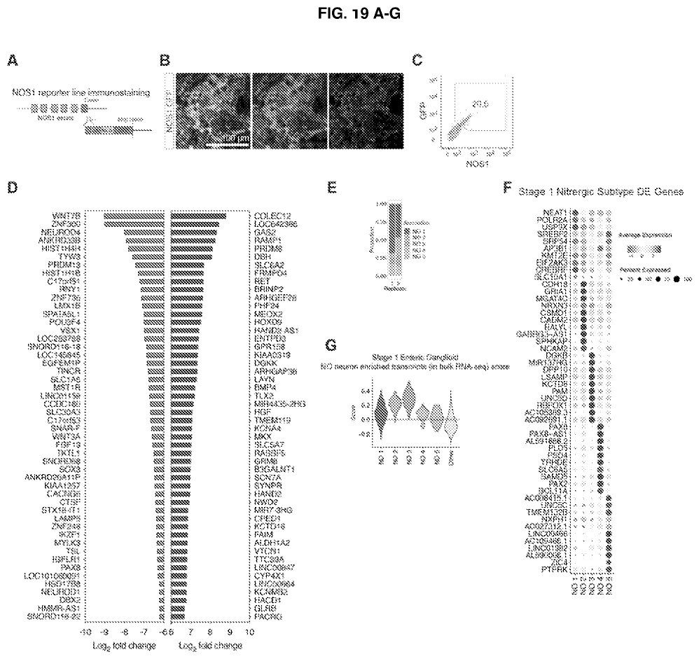

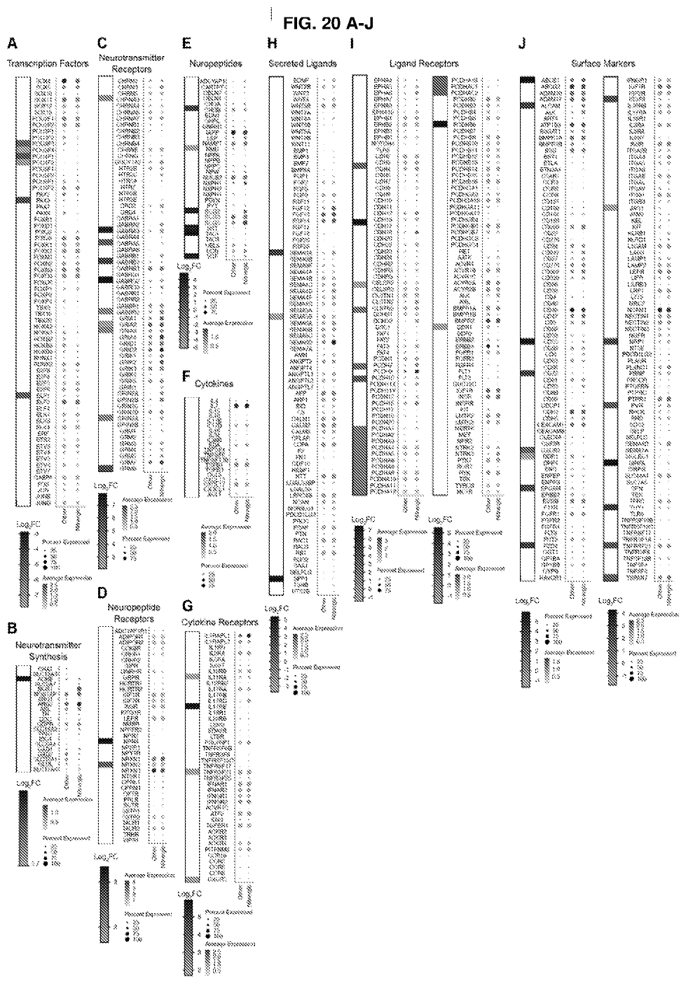

Our hPSC-derived 2D ENS cultures and enteric ganglioids comprise a diverse population of neurons including the NO neurotransmitter identity. Having access to this subtype of neurons prompted us to perform deeper characterization of their molecular and functional identities and develop assays to understand and modulate their activity. To facilitate strategies for studying NO neurons in vitro, we generated a hESC NOS1::GFP line by inserting a GFP cassette under control of the endogenous NOS1 promoter using CRISPR/Cas9 knock-in technique (FIG. 19A). Following our ENS induction protocol NOS1::GFP hESCs gave rise to mature cultures with NO neurons co-expressing GFP and NOS1 (FIGS. 19B and C). We performed bulk RNA-sequencing (bulk RNA-seq) on FACS purified NOS1::GFP⁺/CD24⁺ and NOS1::GFP⁻/CD24⁺, using CD24 as a marker for neurons, and identified differentially expressed genes in NO neurons (FIG. 19D). In parallel, by snRNA-seq profiling of our stage 1 enteric ganglioids, the NO neurons were identified by their expression of the key marker gene, NOS1, and selected metabolic and NO transport genes (FIG. 13A-F, FIGS. 4A and B, Table S1). To determine the molecular diversity within the NO neurons, we performed further sub-clustering and identified 5 subtypes (NO 1-5, FIGS. 4B and C, FIGS. 19E and F). In addition to NOS1, these clusters showed enrichment for other NO biosynthesis pathway genes, confirming their shared NO identity (FIG. 4D). We module scored NO neuron enriched genes identified by bulk RNA-seq in our snRNA-seq clusters and revealed positive enrichment for all NO 1-5, and in particular NO 3, relative to other neurons further confirming the reporter line reliability (FIG. 19G). We assessed the transcriptional profile of a number of gene categories in NO neurons by combining bulk- and snRNA-seq data and revealed many transcription factors, neuropeptides and their receptors, neurotransmitter receptors, cytokines and their receptors, secreted ligands and their receptors and surface markers were differentially expressed in NO neurons relative to other neurons (FIG. 20A-J). The bulk RNAseq data with higher sequencing depth confirmed many of the expression patterns observed in the snRNA-seq. For example, neurotransmitter receptor GABRA3 and neuropeptide SCG2 were enriched, while secreted ligand SEMA3A and ligand receptor DDR1 were depleted in NO neurons (FIGS. 20C, H and I).

Similar to enteric ganglioids, subclustering NO neurons in the adult human primary ENS dataset generated by (Drokhlyansky et al., 2020) identified five primary NO neuron subtypes (pNO 1-5 FIGS. 4E and F). Module scoring revealed similarities between NO 1-5 and pNO 1-5 (FIG. 4G). Similarly, Spearman correlation analysis based on the expression of 3000 anchor features showed correlations for NO 2-5 with pNO 3, as well as NO 2, 4, 5 with pNO 4 (FIG. 4H). Deeper characterization of these subclusters revealed differential expression patterns of some NO neuron specific features (FIGS. 4I and J, Table S2) such as serotonin receptors (HTR2C, HTR5A and HTR3E), GABA receptors (GABRA2 and GABRG1), glutamate receptors (GRIN1, GRIN2A, GRM1 and GRM8), and the opioid receptor OPRM1 (FIGS. 4I and J, Table S2). For example, we checked the expression of surface markers, transcription factors, neuropeptides and their receptors and identified genes that were specific to NO neurons, but showed subcluster specific pattern of expression, such as POU5F1, CARTPT, HTR3E, NPFFR2 and BTLA (FIGS. 4I and J, Table S2). These novel markers of NO neuron subtypes can be utilized for identification and further functional characterizations of unique NO neurons in both our in vitro cultures and from primary tissue samples. We further detected cholinergic, glutamatergic, catecholaminergic, GABAergic and serotonergic identities within the NO neuron subtypes in both datasets (FIG. 4K-M). These data indicate that enteric NO neurons are transcriptionally diverse and may have distinct functional features.

TABLE S2

| | Transcription | | Neurotransmitter | Neuropeptide | Surface |
|---|---|---|---|---|---|
| Neurotransmitter(s) | Factors | Neuropeptides | Receptors | Receptors | Markers |
| Nitrergic | POU5F1 | CARTPT | HTR2C | NPFFR2 | BTLA |
| | | | HTR5A | PRLR | CCR6 |
| | | | GABRA2 | | CD55 |
| | | | GABRG1 | | F11R |
| | | | | | FZD4 |
| | | | | | ITGAL |
| | | | | | TNFRSF8 |
| Glutamatergic | ERF | | | TACR2 | CD8A |
| | HOXB4 | | | | CD72 |
| | PAX6 | | | | CD99 |
| | PAX7 | | | | IL15RA |
| | POU3F4 | | | | LILRB3 |
| | SOX2 | | | | PI16 |
| | | | | | PROCR |
| | | | | | SEMA7A |
| Catecholaminergic | | | GRIK4 | | |
| GABAergic | | SST | GABRG3 | NTSR1 | ALCAM |
| Serotonergic | | | | GIPR | CD38 |
| | | | | TRHR | SELP |
| | | | | | TEK |
| | | | | | TNFRSF13C |
| Nitrergic | | CBLN2 | GABRB1 | | BTN3A1 |
| Serotonergic | | | | | |
| | | | GRM8 | | PTPRC |
| Glutamatergic | | | | | FGFR2 |
| Nitrergic | | | | | |
| GABAergic | | | GRIN1 | | |
| Nitrergic | | | | | |
| | | | GRIN2A | | |
| Cholinergic | | | | NPFFR1 | |
| Serotonergic | | | | | |
| | | | | SSTR1 | |
| Cholinergic | | | GABRA6 | HCRTR2 | DPP4 |
| GABAergic | | | | | |
| Catecholaminergic | | | | | HAVCR1 |
| GABAergic | | | | | |
| Catecholaminergic | | ADCYAP1 | | NPR3 | CDCP1 |
| Nitrergic | | | | | |
| | | TAC1 | | NPY1R | SIRPA |
| | | | | SCTR | |
| Glutamatergic | ELF4 | NPPA | GABRR2 | NPR2 | ANPEP |
| Serotonergic | | | | | |
| | FOXA3 | POMC | GRIN3B | | ART1 |
| | TBX10 | | | | C5AR1 |
| | | | | | CD37 |
| | | | | | CD58 |
| | | | | | CD80 |
| | | | | | CSF1R |
| | | | | | CXCR5 |
| | | | | | F3 |
| | | | | | IL21R |
| | | | | | IL5RA |
| | | | | | LILRA6 |
| | | | | | MELTF |
| | | | | | MRC2 |
| | | | | | NT5E |
| | | | | | SDC2 |
| | | | | | SELPLG |
| | | | | | TNFRSF11A |
| GABAergic | | | GABRA5 | OPRL1 | CD83 |
| Nitrergic | | | GRM1 | TACR1 | ENPP3 |
| Serotonergic | | | | | |
| Catecholaminergic | | | | OPRM1 | |
| Cholinergic | | | | | |
| Nitrergic | | | | | |
| Catecholaminergic | | | | | CD151 |
| Glutamatergic | | | | | |
| Nitrergic | | | | | |
| Glutamatergic | | | CHRNB4 | | |
| Nitrergic | | | | | |
| Serotonergic | | | | | |
| Cholinergic | | | HTR3E | | |
| GABAergic | | | | | |
| Nitrergic | | | | | |
| Serotonergic | | | | | |
| Cholinergic | ETV5 | | | | |
| GABAergic | | | | | |
| Glutamatergic | | | | | |
| Serotonergic | | | | | |
| Catecholaminergic | POU4F2 | | | CRHR2 | |
| Glutamatergic | | | | | |
| Nitrergic | | | | | |
| Serotonergic | | | | | |
| Cholinergic | | | | | CD8B |
| GABAergic | | | | | |
| Glutamatergic | | | | | |
| Nitrergic | | | | | |
| Serotonergic | | | | | |

To determine if these transcriptional differences may reflect differences in localization in the tissue, we used the previously generated neuron-specific myenteric and submucosal gene modules to identify their plexus identity. In the primary dataset, this analysis revealed one highly specific myenteric cluster (pNO 2) and one highly specific submucosal cluster (pNO 4) (FIG. 4N left). Our ganglioid neurons similarly showed alignment with either a myenteric or submucosal identity (FIG. 4N right).

hPSC-Derived ENS Models Identify Modulators of NO Neurons that Promote Colonic Motility

Given the significant role of enteric NO neurons in GI motility and their selective vulnerability in a wide range of congenital and acquired enteric neuropathies (Bódi et al., 2019; Rivera et al., 2011), there has been a great interest in establishing strategies to regulate their function. Factors that modulate NO neuron activity and increase NO release will facilitate the identification of potential drug targets for treatment of enteric neuropathies. Hence, we leveraged our scalable ENS culture platforms to screen for compounds that induce NO neuron activity.

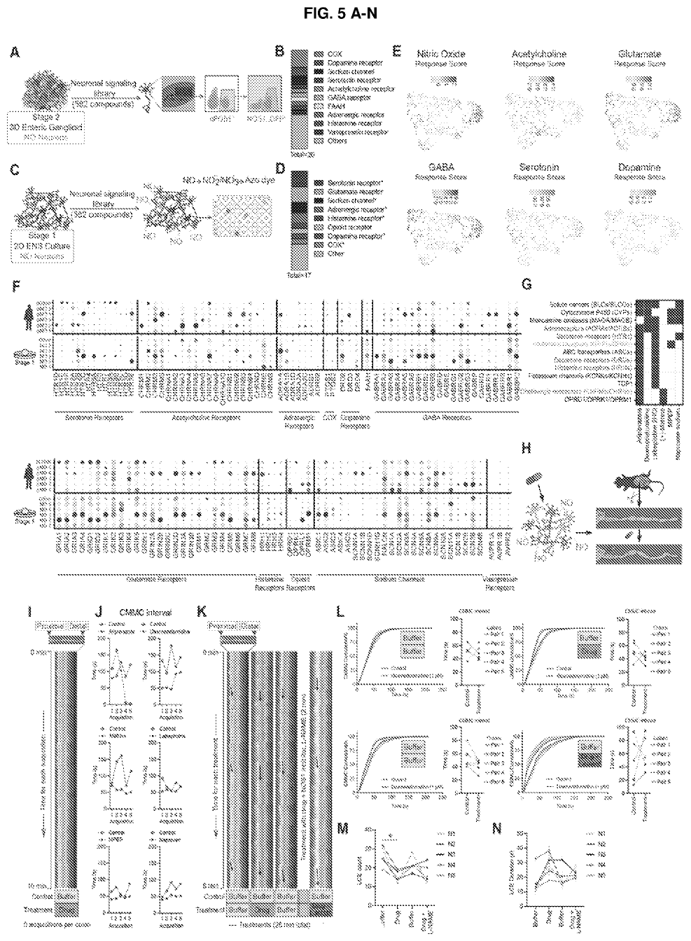

We developed a screening strategy for NO neuron activity based on the induction of cFOS expression as a readout. In order to evaluate cFOS as an accurate read-out of neurochemical-induced activity we performed side by side cFOS flow cytometry analysis and MEA neuronal firing measurements in cultures treated with epinephrine, which is known to stimulate enteric neurons. Epinephrine induced neuronal cFOS expression and resulted in increased electrical firing of ganglioid neurons. This provides a scalable read-out of activity that is suitable for high-throughput screens. (FIG. S20A-C). FIG. S20 data not shown, but it describes identifying enteric NO neuron modulators by functional high-throughput screenings. The data of S20 were disclosed in Majd et al., “hPSC-Derived Enteric Ganglioids Model Human ENS Development and Function”, bioRxiv, posted Jan. 3, 2022, which is incorporated by reference in its entirety. First, we performed a cFos induction screen, where NOS1::GFP enteric ganglioid cells were exposed to a library of 582 neuromodulators (Selleck neuronal signaling library™) and co-expression of cFOS and GFP was measured to quantify NO neuron activity (FIG. 5A, FIG. S20D). We identified 20 compounds that increased the proportion of NO neurons in cFOS* cells with z-score>1.5. To identify the mechanisms involved in cFOS expression in NO neurons, we compiled and classified the list of target proteins and discovered multiple shared protein classes. In particular, these proteins converged on serotonin receptors, sodium channels, acetylcholine receptors, glutamate receptors, adrenergic receptors, histamine and opioid receptors and dopamine receptors (FIG. 5B).

In an independent functional screen, we established a high-throughput read-out for assessing NO neuron activity. We utilized a commercially available kit that enables NO detection in the media. Upon release into the media, NO is spontaneously oxidized to nitrate. The kit uses nitrate reductase to convert nitrate to nitrite that is then detected as a colored azo dye. We incubated 2D ENS cultures with the neuromodulators library and measured NO release using calorimetry (FIG. 5C). We identified 17 compounds that remarkably enhanced NO concentration in the supernatant with z-score>2.0 (FIG. S20E). Neuromodulators that induced NO release in our ENS cultures were diverse but were predicted to commonly target protein classes including serotonin receptors, sodium channels, acetylcholine receptors, glutamate receptors, adrenergic receptors and opioid receptors (FIG. 5D).

Interestingly, there was a high degree of similarity between the predicted targets from the cFOS induction and NO release screens (FIGS. 5B and D, FIG. S20F). These targets included receptors for neurotransmitters such as serotonin and dopamine. Module scoring the neurotransmitter receptor gene families in our hPSC-derived stage 1 enteric NO neurons snRNA-seq data confirmed that NO neurons broadly express receptors for NO, serotonin, GABA, glutamate, acetylcholine or dopamine (FIG. 5E). Interestingly, compared to other neuronal subtypes, stage 1 NO neuron clusters were enriched for all predicted hit targets (FIG. S20G). In particular, NO 3 cluster scored high for the expression of the majority of NO neuron modulator target classes (FIG. S20H). Profiling the expression of individual genes in each target protein category in ganglioids and primary human ENS revealed notable subtype-specific expression patterns between NO neurons. For example, GABA receptor genes were predominantly expressed by NO 2, NO 3 and pNO 4 subtypes, while the expression of acetylcholine receptor genes was less specific to a particular subtype (FIG. 5F).

We then selected a subset of hits representing different target classes, prioritizing FDA approved compounds for follow up analyses (FIG. 5K, FIG. S20F). For selected compounds, we performed a more comprehensive and integrated target analysis by combining reported experimental data (Binding DB) and computational methods (SEA, Carlsbad, Dinies, Swisstarget, Superdrug, Pubchem Bioassays, FIG. 5G). We next tested the effects of these compounds on colonic motility in organ bath assays (FIG. 5H). In these assays, we maintained resected segments of mouse colon in a physiologic buffer to study motility patterns using video recording. In the first experiment we tested the effect of all selected drug candidates on mouse colonic motility, ex vivo (FIG. 5I). In each experiment, we tested an untreated control and a drug-treated colon sample side by side during five consecutive 10-min acquisitions. For each acquisition, instead of analyzing fecal outputs, which are generally variable, we performed more sophisticated contraction analysis by generating spatiotemporal maps from video data based on the changes in the colonic diameter over time, and used to calculate the rate of colonic migrating motor complexes (CMMC) and slow waves (SW). CMMCs are rhythmic propulsive contractions initiated by the ENS while SWs are mediated through the pacemaking activity of the interstitial cells of Cajal (Barajas-López and Huizinga, 1989; Burns et al., 1996; Fida et al., 1997; Lyster et al., 1995; Smith et al., 1987). To probe the dynamics of CMMC and SW events during each acquisition, we generated cumulative percent graphs (data not shown) and calculated the intervals at the 75th percentile (FIG. 5J, data not shown). Compounds that showed promising effects on lowering CMMC intervals relative to the untreated condition were chosen for follow-up assessment (i.e. aripiprazole, dexmedetomidine, matrine, MPEP) (FIG. 5J, data not shown). To assess whether the compounds mediated their effect on CMMCs through modulating NO release, we performed sequential drug treatments in the presence and absence of the NOS1 inhibitor N(omega)-nitro-L-arginine methyl ester (L-NAME). Each experiment consisted of four 6-min acquisitions on five independent sample pairs in the control and drug-treated groups (FIG. 5K). Of the tested drugs, the adrenergic receptor agonist dexmedetomidine, decreased CMMC intervals in four out of five colon samples, an effect that was absent when colons were treated with dexmedetomidine+L-NAME simultaneously (FIG. 5L, data not shown). SWs were not affected by the drug treatment (Data Not Shown). In addition to CMMCs, we quantified the effects of compounds on colonic motility by annotating and measuring anterograde contractile events detected in the spatiotemporal map. These events were termed “longitudinal contractile events” (LCE) and highlighted by representative arrows in FIG. 5K. In dexmedetomidine-treated colons, we observed a decrease in the total number of LCEs (FIG. 5M) and an increasing trend in their average duration (FIG. 5N) in all five replicates. These effects were reversed after the drug removal and blocked by L-NAME co-treatment (FIGS. 5M and N). These results provide a blueprint for leveraging in vitro human ENS models to uncover mechanisms that regulate GI motility which are capable of identifying therapies that target specific ENS populations.

### High-Throughput Small Molecule Screen Reveals PDGFR Inhibition as a Driver of NO Neuron Induction

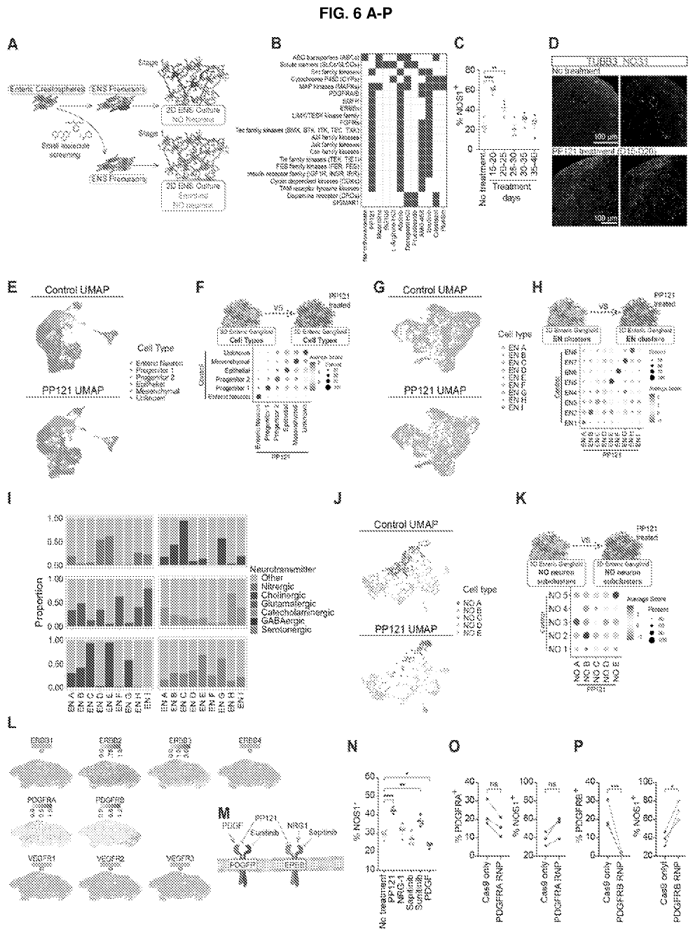

To evaluate the potential of hPSC-derived cultures to model human ENS development, we set out to define the mechanism of NO neuron specification in vitro. Searching for pathways that regulate NO neuron differentiation, we performed a high-throughput small molecule screen. Identifying distinct pathways and chemical modulators that promote NO neuron induction could provide insights on NO neuron development and offer a strategy for the derivation of NO neuron enriched ENS cultures.
To identify compounds that induce NO neuron differentiation, we treated enteric crestospheres with 1694 compounds in the Selleck inhibitor library™ and identified 12 hit compounds that increased the proportion of NOS1⁺ neurons by at least eight folds (FIG. 6A, FIGS. S25A and B). FIG. S25 data not shown, but it describes that small molecule high-throughput screening identifies compounds that enrich NO neurons in hESC-derived ENS cultures. The data of S25 were disclosed in Majd et al., “hPSC-Derived Enteric Ganglioids Model Human ENS Development and Function”, bioRxiv, posted on Jan. 3, 2022, which is incorporated by reference in its entirety. In order to reveal the mechanisms by which these hit compounds enhanced NO neuron induction, we performed target prediction analysis by combining reported experimental data (Binding DB) and computational methods (SEA, Carlsbad, Dinies, Swisstarget, Superdrug, Pubchem Bioassays) (FIG. 6B). After clustering the predicted protein targets, common patterns emerged for a subset of compounds. For example, PP121, ibrutinib, afatinib, and AMG-458 were all predicted to interact with EGFR, ERBBs, MAP, and TEC family kinases among others (FIG. 6B). For follow-up analysis, we chose to focus on PP121, the hit with the highest % NOS1⁺ fold increase in this subset of compounds. PP121 showed a dose-dependent effect on NO neuron induction efficiency as measured by flow cytometry (FIG. S25C). To find the most effective treatment window for PP121-induced NO neuron induction, we treated the differentiating cultures for five days at various time points. Measuring GFP signal in stage 1 NOS1::GFP enteric ganglioids showed the highest induction efficiency for cells treated during day 15-20 (FIGS. 6C and D, FIG. S25D).

For the enrichment protocol to be reliable, it was important to confirm that PP121 treatment did not change the identity of our cell types. To compare PP121 treated and untreated stage 1 enteric ganglioids in single cell resolution, we performed snRNA-seq and combined both datasets. This analysis revealed that all cell types were represented in both conditions (FIGS. 6E and E, FIG. S26A). FIG. S26 data not shown, but it describes PP121 treatment enriches NO neurons without affecting their overall cellular diversity. The data of S26 were disclosed in Majd et al., “hPSC-Derived Enteric Ganglioids Model Human ENS Development and Function”, bioRxiv, posted on Jan. 3, 2022, which is incorporated by reference in its entirety. Importantly, comparison of the average expression of all genes for matched PP121 treated and untreated cell types showed highly similar transcriptomes (R2 correlations >0.91), indicating that PP121 treatment did not change the transcriptional identity of cell types (FIG. S26B). Interestingly, sub-clustering of the merged control and PP121 treated neurons revealed nine neuronal subtypes EN A-I (FIG. 6G). The PP121 treated dataset subtypes showed high transcriptional similarity to EN 1-8 of the control only dataset (FIG. 6H). EN cluster I consisted of mostly PP121 treated cells and few control cells and showed moderate transcriptional similarity to the control only EN cluster 4, suggesting that this neuronal subtype is present but rare in control cultures causing those neurons to cluster with the most similar subtype, EN 4 (FIG. 6H, FIG. S26C). Along with EN I which is roughly 25% nitrergic, PP121 treatment also enriched cultures for neuronal subtypes EN D and H (roughly 50% and 25% nitrergic, respectively), while EN A and G were less represented (FIG. 6I, FIG. S26C). Again, despite the changes in subtype abundance, control and PP121 treated neurons of the same subtype showed similar transcriptomes (R2 correlations >0.88) (FIG. S26D). Further sub-clustering of merged control and PP121 treated nitrergic neurons revealed an enrichment for Nitrergic B (most similar to control only Nitrergic 2) and a rare population, Nitrergic C (most similar to control only Nitrergic 3) (FIGS. 6J and K, FIG. S26E). Transcriptome comparison again showed highly similar gene expression of control and PP121 treated nitrergic neurons of the same subtype (R2 correlations >0.7) with the highest variance between Nitrergic C neurons, likely due to the small number of neurons in this cluster (FIG. S26F). Altogether, this data suggests that early treatment of ganglioids with PP121 causes changes in the abundance of neuronal subtypes normally found in untreated cultures without affecting the gene expression patterns of the subtypes that arise.

The ability to purify enteric NO neurons is of great interest especially for applications such as cell therapy. Access to NOS1::GFP reporter line and the ability to direct the differentiation towards NO neurons using PP121, allowed us to search for FACS-compatible surface markers for these cells. We screened a panel of 242 antibodies for human cell surface molecules (BD lyoplate) and measured GFP and surface antigen expression signals by flow cytometry (FIG. S27A). FIG. S27 data not shown, but it describes that huma surface marker antibody screening identifies NO neuron specific surface markers. The data of S27 were disclosed in Majd et al., “hPSC-Derived Enteric Ganglioids Model Human ENS Development and Function”, bioRxiv, posted Jan. 3, 2022, which is incorporated by reference in its entirety. We identified 27 antibodies that stained at least 50% of NOS1 neurons (% CD⁺GFP⁺ in GFP⁺ population, FIG. S27B top). FIG. S27 data not shown, but it describes that human surface marker antibody screening identifies NO neuron specific surface markers. The data of S27 were disclosed in Majd et al., “hPSC-Derived Enteric Ganglioids Model Human ENS Development and Function”, bioRxiv, posted on Jan. 3, 2022, which is incorporated by reference in its entirety. To identify the most specific candidates among these hits, we looked for antibodies with a >70% CD⁺GFP⁺ to CD⁺ staining ratio (FIG. S27B bottom). CD47, CD49e, CD59, CD90, and CD181 met both criteria (FIG. S27C). We further confirmed the expression and enrichment of CD47, CD49e, CD59 and CD90 in stage 1 ganglioid and NO neuron clusters in the human primary snRNA-seq dataset (FIGS. S27D and E). As an example, we further confirmed the localization of CD47 in NO neurons in human primary colonic myenteric ganglia using immunohistochemistry (FIG. S27F). In addition to identifying antibodies to specifically enrich NO neurons, we found twelve antibodies that stained >70% of ganglioid cells and could serve as pan enteric neuronal surface markers (CD24, CD45RA, CD57, CD63, CD71, CD121b, CD147, CD164, CD184, CD193, CD243, CD275) (FIG. S27G). We confirmed the enriched expression of CD24 in our enteric neurons (snRNA-seq data) and also primary human colon myenteric ganglion (FIGS. S27 H and I).

To determine the mechanism by which PP121 induced NO neuron enrichment in ganglioids, we used a combination of pharmacological and genetic approaches. PP121 is a multi-targeted receptor tyrosine kinase (RTK) inhibitor with known inhibitory activity on PDGFRs, VEGFRs and EGFRs (Apsel et al., 2008). Our crestosphere snRNA-seq analysis confirmed the expression of PDGFRA, PDGFRB, ERBB2 and ERBB3 while the mRNA for VEGFRs were not detectable (FIG. 6L). We evaluated the induction efficiency of NO neurons in response to PDGF (PDGFR agonist), sunitinib (PDGFR and VEGFR antagonist), NRG1 (ERBBs agonist) and sapitinib (ERBBs antagonist) (FIG. 6M). While NRG1 and sapitinib showed no significant effect on NO neuron induction, treatment with PDGF and sunitinib led to lower and higher NO neuron proportions respectively (FIG. 6N). To genetically confirm the role of PDGFR signaling in NO neuron induction, we used CRISPR-Cas9 to knock-out PDGFRA and PDGFRB in our enteric crestospheres and analyzed the percentage of NO neurons in stage 1 ganglioids. NO neurons were enriched in both PDGFRA and PDGFRB knock-out cultures further confirming the PP121 mechanism of action (FIGS. 6O and P).

hESC-Derived NOS1 Neurons Engraft in Nos1^{31 /-} Mouse Colon

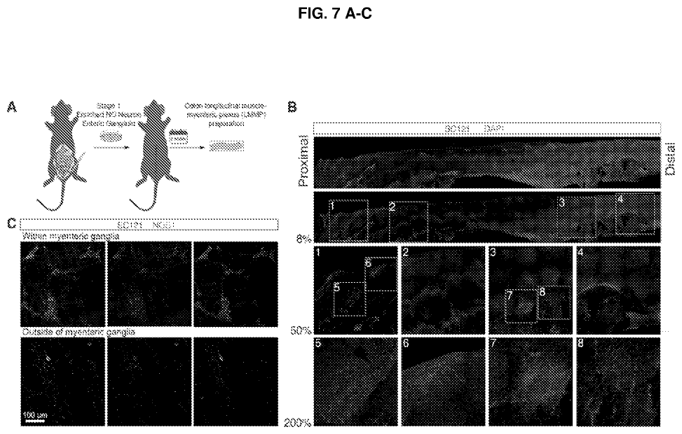

Developing an experimental system to study the human ENS in vivo opens a wide range of basic science and clinical opportunities. For example, human ENS xenografts will enable studying human neuronal circuitry in vivo and investigating ENS-CNS and ENS-immune system-microbiome communications. They also provide platforms for disease modeling and drug development. In addition, the limited regenerative capacity of the ENS highlights the importance of developing cell therapy approaches to replace the lost populations of neurons. There is currently no clinical intervention to replace the damaged or lost neurons caused by genetic and acquired ENS pathologies such as Hirschsprung disease and diabetes. We have previously shown hPSC-derived ENC precursors can successfully engraft in vivo (Fattahi et al., 2016). McCann et. al. have also shown the transplanted ex vivo cultured murine enteric neurospheres are able to rescue GI motility defects in Nos1−/− mice (McCann et al., 2017). However, these neurospheres are heterogeneous populations containing only a small percentage of NO neurons. Additionally, obtaining sufficient numbers of neurospheres from human primary tissue poses a significant limitation for ultimate regenerative applications. Compared to ENC precursors, transplanting mature neurons provides a post-mitotic source of cells with a lower clinical risk of tumor formation. Obtaining highly enriched NO neuron cultures encouraged us to assess the transplantation potential of our enteric ganglioids. PP121 treated enteric ganglioids were injected in the wall of distal colon in immunocompromised Nos1−/− (B6.129S4-Nos1tm1Plh/J) mice. Animals were sacrificed eight weeks post-surgery and colonic longitudinal muscle myenteric plexus (LMMP) preparations were assessed by fluorescence microscopy (FIG. 7A). Transplanted cells were distinguished by the expression of human cytoplasmic marker SC121. Notably, we observed a remarkable number of SC121+ cells that had integrated along the length of the colon (FIG. 7B). Engrafted cells were detected within, and outside of myenteric ganglia and many expressed NOS1 confirming their NO fate (FIG. 7C, FIG. S28). FIG. S28 data not shown, but it describes h-ESC-derived enteric ganglioids engraft in adult mouse colon. The data of S28 were disclosed in Majd et al., “hPSC-Derived Enteric Ganglioids Model Human ENS Development and Function”, bioRxiv, posted Jan. 3, 2022, which is incorporated by reference in its entirety. In addition to the clinically-significant cell therapy application, the developed human enteric ganglioid xenograft offers previously unachievable opportunities towards understanding development, physiology and pathophysiology of the human ENS in vivo.

### Discussion

The ENS is a complex network of enteric neurons and glia that controls all aspects of GI physiology (Long-Smith et al., 2020; Schneider et al., 2019; Yoo and Mazmanian, 2017) and plays a central role in initiation and progression of enteric neuropathies and diseases of the gut-brain axis (Camilleri, 2021; Niesler et al., 2021; Pesce et al., 2018). Nevertheless, our understanding of the ENS has been disproportionately affected by long standing technical challenges. Gaining access to human ENS requires invasive biopsies or surgeries as these cells only comprise 1% of the gut tissue (Drokhlyansky et al., 2020) and reside deep within muscular and mucosal layers. Moreover, large scale isolation and purification of ENS cells is extremely challenging. The majority of neuronal cell bodies are positioned in ganglia with their fragile projections extending to other parts of the gut tissue. In addition, there are no well-established surface markers for FACS-based purification of specific subtypes of enteric neurons or glia. Furthermore, animal models do not fully recapitulate the human ENS (patho) physiology. For example, rodents can well tolerate mutations that cause life-threatening enteric neuropathies in humans (Bondurand and Southard-Smith, 2016). Here, we present hPSC-derived ENS cultures as alternative models that overcome many of these challenges and enable major advances in the field of enteric neurobiology.

We thoroughly compared the composition of the hPSC-derived ENS platform against the recently published primary ENS datasets by snRNA-seq (Drokhlyansky et al., 2020; Morarach et al., 2021) and revealed diverse neuronal and glial subtypes that resemble the cellular diversity found in vivo. For example, the hPSC-derived enteric neurons express key markers and receptors for numerous hormones, neuropeptides and neurotransmitters that are known to exist in primary human ENS supporting the reliability and utility of our hPSC-based platform for modeling the human ENS. By clustering and further sub-clustering of our datasets we identified novel markers for each subtype offering opportunities for immunochemistry-based detection, purification, genetic manipulation, and reporter line development. Furthermore, investigation of the primary and hPSC-derived neurons shed light on the array of neurons with multiple neurochemical identities. These neurochemically diverse neurons greatly outnumber the ones that align with the traditionally and widely accepted belief that one-neuron expresses one-neurotransmitter. Although there have been immunohistochemical based reports of enteric neurons with multiple neurochemical identities (Qu et al., 2008), a comprehensive characterization has not been carried out before. The level of complexity revealed here could not be easily recognized and characterized by common lower-throughput staining based detection methods. This is of high scientific and medical value, furthering our understanding of the human ENS circuitry and autonomy, and informing the development of more targeted therapeutics with fewer side effects.

The autonomy of the ENS and its ability to perform diverse tasks independently relies on the diversification of its neuronal and glial components through elaborate fate specification processes. The precise developmental patterns that drive the differentiation of vagal neural crest into ENS progenitors that consequently mature into a myriad of neuronal and glial subtypes has remained elusive particularly in humans. Studying these complicated developmental patterns is extremely challenging due to the transient nature of many of the developmental states, technical limitations of isolating the tissue, and inter-species differences. Leveraging our stepwise ENS induction system, we generated high-resolution temporal maps revealing the complicated developmental programs that give rise to enteric neuron and glia. We begin the in vitro differentiation by inducing vagal and enteric neural crest that develop into enteric crestospheres, which we further differentiate into enteric neurons and glia. Interestingly, in long-term cultures we observed the appearance of enteric glial subclasses that resemble adult human primary glia. This resembles the developmental timeline in the CNS, where gliogenesis follows neurogenesis. Given the complexity of the processes influencing ENS development, it is not surprising that defects at any developmental stage lead to enteric neuropathies such as Hirschsprung disease (Lake and Heuckeroth, 2013; Rao and Gershon, 2018). Investigating the broad and cell-type specific developmental programs in the ENS provides an opportunity for understanding developmental neuropathies and facilitates the directed derivation of disease-relevant cell types.

An exceptional advantage of hPSC-derived cultures is their scalability. This is particularly important when the desired cell types are rare, and have very limited regenerative and proliferative capacity such as nervous tissue. Our ENS culture platforms have repeatedly proven to be reliable in providing scalable sources of ENS cell types that are compatible with applications that would otherwise be extremely challenging to implement, such as high-throughput screens. In particular, using our 2D ENS cultures we screened thousands of inhibitors to identify compounds that direct the differentiation towards the clinically valuable NO neurons. Investigating the mechanism of action of our top hits revealed pathways that are important in NO neuron fate specification. Using a combination of pharmacological and genetic approaches, we discovered the contribution of one such pathway, PDGFR signaling, in inducing NO neurons, which highlights the remarkable potential of hPSC-based platforms to uncover developmental mechanisms.

Our 2D and 3D ENS cultures are electrically active. Access to functional enteric neurons is extremely advantageous as it facilitates the basic understanding of neuronal circuits and cellular electrophysiology. Further, identifying cell-type specific neurochemical and functional characteristics is invaluable in drug development as it enables the identification of targeted neuromodulators and prediction of potential side effects through direct and indirect neurochemical mechanisms. As a proof of concept, we developed functional screening platforms to uncover candidate drugs that specifically modulate the activity of NO neurons. Interestingly, our hit compounds commonly target adrenergic, cholinergic and serotonergic receptors and sodium channels. Notably, these targets are overrepresented in NO neurons, highlighting the specificity of these compounds and their potential for further therapeutic development for GI indications. By testing a subset of these neuromodulators, we further demonstrated that these candidate drugs are capable of affecting colonic motility patterns in ex vivo organ bath assays. This is the first example of identifying candidate drugs for modulating GI motility by targeting a specific enteric neuron subtype. These findings showcase the reliability, robustness, and scalability of our hPSC derived ENS models.

Derivation of enteric ganglioids from hPSCs provides a scalable source of human ENS tissue for regenerative applications. Additionally, developing human ENS xenografts opens a wide range of basic science and clinical research avenues. In the last two decades, developing cell-based therapies for enteric neuropathies has been a major area of research (Alhawaj, 2021; Burns et al., 2016). However, a scalable source of human ENS cells suitable for transplantation is challenging to achieve. Here, we provide proof of concept results on extensive engraftment of NO neurons in Nos1^{-/-} mice by transplanting ganglioids enriched for this neuronal subtype. Beyond cell therapy, these human ENS xenograft models provide a new experimental system for various purposes. First, these models enable the study of human ENS in vivo and facilitates the identification and development of therapeutic candidates with high specificity, efficacy and potency. Second, they may be used models to study human ENS pathologies in vivo, using strategies such as transplanting ganglioids harboring specific mutations, ganglioids exposed to specific stressors, or ganglioids derived from patient iPSCs. Third, transplanting ganglioids at different stages of differentiation enables comprehensive studies on cell fate specification and maturation in the human ENS. Finally, human ENS xenografts offer promising models for studying the crosstalk between the human ENS and local gut tissues, the CNS, and the microbiome.

Enteric neuropathies can affect any part of the GI tract at any stage of life, and represent some of the most challenging clinical disorders with no effective therapies. They can result from congenital defects affecting ENS development, can occur in response to changes in the tissue environment (toxins, microbes, immune system), or can emerge secondary to systemic diseases such as diabetes and obesity (Camilleri et al., 2011; Niesler et al., 2021; Yarandi and Srinivasan, 2014). The lack of efficient therapies stems from our inadequate understanding of ENS development, cellular architecture, and function. Our hPSC-derived 2D ENS cultures and enteric ganglioids provide human-based platforms to model enteric neuropathies. Genetic manipulation of neurons, glia, and their specific subtypes at different stages of their development is now possible. The effect of genetic background (healthy and patient-derived iPSCs) (Lai et al., 2017) and specific mutations as well as environmental stressors, infectious agents, metabolic toxins can now be studied via targeted and unbiased approaches. Furthermore, functional and fully characterized ENS cultures open avenues for investigating additional layers of complexity represented in gut physiology, such as crosstalk with the surrounding and distant tissues. For example, we can study motor functions by developing co-cultures with smooth muscle cells; investigate ENS-immune system communication by setting up co-cultures with immune cells, and interrogate ENS-gut microbiome interactions by exposing ENS cells to gut microbiome by-products.

Our hPSC differentiation strategy provides robust 2D and 3D ENS culture systems that enable developmental, molecular and functional mapping of human ENS. We provide key insights into physiological properties of NO neurons and identify the developmental programs required to specify this clinically relevant enteric neuron subtype. These models open up new avenues for drug discovery and regenerative medicine and offer a new framework for basic studies of enteric neurobiology.

### Methods

### Culture and Maintenance of Undifferentiated Human Stem Cells

Human embryonic stem cell (hESC) line H9 (WAe009-A, and reporter expressing derivatives hSYN::ChrR2-EYFP, NOS1::GFP) and induced pluripotent stem cell (hiPSC) line WTC-1 1 (UCSFi001-A) were plated on geltrex™-coated plates and maintained in chemically-defined medium (E8) as described previously (Barber et al., 2019). The maintenance cultures were tested for mycoplasma every 30 days.

### Enteric Neural Crest (ENC) Induction

When the monolayer cultures of hPSCs reached about 70% confluency, a previously established 12-day enteric neural crest (ENC) induction protocol was initiated (Barber et al., 2019; Fattahi et al., 2016) (DO) by aspirating the maintenance medium (E8) and replacing it with neural crest induction medium A [BMP4 (1 ng ml⁻¹), SB431542 (10 μM), and CHIR 99021 (600 nM) in Essential 6 medium]. Subsequently, on ENC induction days D2 and D4, neural crest induction medium B [SB431542 (10 μM) and CHIR 99021 (1.5 μM) in Essential 6 medium] and on D6, D8, and D10 medium C [medium B with retinoic acid (1 μM)] were fed to the cultures. Next, ENC crestospheres were formed during D12-D15 to facilitate the selection for ENC lineage and against contaminating ones in our cultures. In doing so, we removed ENC induction crest medium C on D12 and detached the ENC monolayers using accutase (30 min, 37° C., 5% CO₂). After centrifuging the samples at 290×g for 1 min, we re-suspended the ENC cells in NC-C medium [FGF2 (10 ng ml⁻¹), CHIR 99021 (3 μM), N2 supplement (10 μl ml⁻¹), B27 supplement (20 μl ml⁻¹), glutagro (10 μl ml⁻¹), and MEM NEAAs (10 μl ml⁻¹) in neurobasal medium] and transferred them to ultra-low-attachment plates to form free-floating 3D enteric crestospheres. On D14, when the free-floating enteric crestospheres could be observed, we gently gathered them in the center of each well using a swirling motion. Then, the old media was carefully aspirated from the circumference of each well without removing the crestospheres. After addition of the fresh NC-C medium, the cultures were incubated for 24 hours (37° C. and 5% CO₂) prior to enteric neuron induction phase.

Enteric Neuron Induction from Enteric Neural Crests

On D15, enteric crestospheres were gathered in the center of the wells using a swirling motion and NC-C medium was removed using a P1000 micropipette in slow circular motion, avoiding the free-floating crestospheres. At this step protocol varied depending on the final desired culture layout (2D ENS cultures versus 3D enteric ganglioids). For 2D ENS cultures, after washing the enteric crestospheres with PBS, accutase (Stemcell Technologies, 07920) was added and plates were incubated for 30 minutes at 37° C. to dissociate the crestospheres. Then, remaining spheroids were broken by pipetting ENC medium [GDNF (10 ng ml⁻¹), ascorbic acid (100 μM), N2 supplement (10 μl ml⁻¹), B27 supplement (20 μl ml⁻¹), glutagro (10 μl ml⁻¹), and MEM NEAAs (10 μl ml⁻¹) in neurobasal medium]. Cells were spun (2 min, 290×g, 20-25° C.) and supernatant was removed. Pellet was resuspended in ENC medium and cells were plated on poly-L-omithine (PO)/laminin/fibronectin (FN) plates at 100,000 viable cells per cm². For 3D enteric ganglioids, we avoided accutase treatment and enteric crestospheres were fed with the same volume of ENC medium [GDNF (10 ng ml⁻¹), ascorbic acid (100 μM), N2 supplement (10 μl ml⁻¹), B27 supplement (20 μl ml⁻¹), glutagro (10 μl ml⁻¹), and MEM NEAAs (10 μl ml⁻¹) in neurobasal medium]. Feeding continued every other day with ENC medium until D30-D40, after which, feeding frequency could be reduced to once or twice per week but with a larger volume of feeding medium.

### Immunofluorescence

For immunofluorescence (IF) staining, cells were initially fixed in 4% PFA in PBS (30 min, room temperature (RT), and then blocked and permeabilized by permeabilization buffer (PB) (Foxp3/Transcription Factor Staining Buffer Set, 00-5523) for another 30 minutes at RT. After fixation and permeabilization steps, cells were incubated in primary antibody solution overnight at 4° C., and then washed three times with PB before their incubation with fluorophore-conjugated secondary antibodies at RT. Before imaging, stained cells were incubated with DAPI fluorescent nuclear stain and washed an additional three times. The list of antibodies and working dilutions is provided in Table S3.

TABLE S3

| Antibody | Host | Vendor | cat number | used for | dilution |
|---|---|---|---|---|---|
| cFOS | rabbit | Abcam | ab190289 | IF, FC | 1:2500, 1:2500 |
| GFP | chicken | Abcam | ab13970 | IF, FC | 1:1000 |
| NOS1 | mouse | Santa Cruz Biotechnology | sc-5302 | IF, FC | 1:50 (IF), 1:100 |
| NOS1 | rabbit | Invitrogen | 61-7000 | FC | 1:200 |
| CHAT | rabbit | Proteintech | 20747-1-AP | IF | 1:1000 |
| CHAT | goat | Sigma | AB144P | FC | 1:1000 |
| 5HT | rabbit | Sigma | s5545 | IF, FC | 1:8000 |
| 5HT | goat | immunostar | 20079 | FC | 1:2500 |
| GABA | rabbit | Sigma | a2052 | IF, FC | 1:2500 |
| GABA | ms IgG1 | Sigma | A0310 | FC | 1:100 |
| GFAP | chicken | abcam | ab46674 | IF, FC | 1:2500 |
| TUBB3 | chicken | Millipore Sigma | ab9354 | FC | 1:350 |
| TUBB3 | mouse IgG2a | Biolegend | 801202 | IF | 1:1500 |
| GFAP | chicken | abcam | ab4674 | IF | 1:2500 |
| PMP22 | rabbit | abcam | ab203053 | IF | 1:100 |
| MPZ | chicken | abcam | ab39375 | IF | 1:50 |
| S100 | rabbit | Thermo Scientific | RB-9018-P0 | IF | 1:500 |
| GYPB (CD235a) | mouse IgG2b | BD bio | 555569 | FC | 1:250 |
| CCR6 | R&D msIgG2b | R&D | MAB195 | FC | 1:500 |

### Preparation of Enteric Ganglioid Frozen Sections

hPSC-derived ganglioids were collected at stage 1 (day 37-50) and stage 2 (day 70-90), rinsed twice in PBS and fixed on ice in 4% PFA (SCBT sc-281692) for 3 hours, followed by replacing 90% of the supernatant with PBS for storage at 4° C. for up to 6 months. Ganglioids were treated with 5% sucrose (RPI Research Products 524060) in PBS for 10 minutes at room temp, followed by 10% sucrose in PBS for 2 hours at room temp and 20% sucrose at 4° C. overnight. Sucrose-treated ganglioids were positioned in cryomolds (Tissue-Tek® Cryomold® medium, VWR 25608-924), all 20% sucrose removed and incubated in 2:1 20% sucrose:OCT (Tissue Plus O.C.T. Compound Fisher HealthCare 5484) for 2 hours at room temperature before flash freezing in ethanol/dry ice. 1220 μm sections were taken on a cryostat (Leica 3050S) adhered to Superfrost® Plus Micro Slide, Premium (VWR 48311-703) and dried on 42° C. slide dryer for up to 2 hours before storing at −80° C. for up to a year.

### Preparation of Paraffin-Embedded Human Colon Sections

Human sigmoid colon tissue was received from the International Institute for the Advancement of Medicine (IIAM) that provides non-transplantable organs from Organ Procurement Organizations for biomedical research purposes. Colon tissue was obtained under sterile conditions, flushed with isotonic solution, submerged in organ transplant solution, and shipped on ice to laboratory within 24 hours post mortem. Full-thickness tissues pieces (˜2 cm2) were fixed overnight (<24 hours) in 10% neutral buffered formalin (Cancer Diagnostics, FX1003). Samples were transferred to 70% ethanol prior to paraffin embedding (Leica ASP6025, tissue processor). Approximately 5 μM thick transverse tissue sections were cut onto coated glass slides (Superfrost® Plus Micro Slide; VWR, 48311-703) and air-dried overnight. All following slide preparation steps were performed at room temperature. Slides with paraffin sections were washed three times in clean xylene substitute (Sigma A5597), then once each in 100% ethanol, 95% ethanol, and 70% ethanol. Slides were then run under house DI water for 5 minutes before being placed in 1×PBS for storage at 4° C. for up to 4 weeks. Prior to staining, paraffin sections underwent antigen retrieval in either citrate buffer (Vector Laboratories Antigen Unmasking Solution H-3300) or TE buffer (Thermo 17890, brought to pH 9.0 with 1 M NaOH). Slides were incubated in buffer for 10 minutes at 95° C. using a Pelco BioWave Pro+ set to 400 watts.

### Staining Enteric Ganglioid Frozen Sections and Paraffin-Embedded Human Colon Sections

Unless otherwise specified, all steps were performed at room temperature. Ganglioid frozen sections and paraffin-embedded human normal colon sections were prepared as above and then washed three times in PBS and blocked for 1-2 hours in serum (10% donkey or 10% goat) with 0.5% (v/v) Triton X-100 (VWR 0694). Slides were then incubated with primary antibody diluted in serum (10% donkey or 10% goat) with 0.1% Triton X-100 at 4° C. for 12-20 hours. Slides were washed six times for 20 minutes each in PBS with 0.1% Tween-20 (Sigma P1379) and incubated for 1 hour with Alexa Fluor conjugated secondary antibodies. The diluted secondary antibody solution was removed and replaced with 1.0 μg/mL DAPI in water for 10 minutes. The slides were washed six times for 20 minutes each in PBS with 0.1% Tween-20 and coverslips were mounted with Fluoromount-G (Southern Biotech 0100-01). The list of antibodies and working dilutions is provided in Table S3. Images were acquired on a Leica SP8 inverted confocal or on the Echo Revolve. For images that were stitched we used Leica's LAS X tiling feature or the Grid/Pairwise stitching plugin for FIJI (PMID 19346324).

### 2-Photon Fluorescence Imaging

Imaging experiments were conducted on a custom-built upright 2-photon microscope operating with μManager software (San Francisco, CA). The excitation source was a 2-photon Coherent Chameleon Vision II laser operating at 760 nm (Coherent, Santa Clara, CA). Images were collected using an Olympus LWD 1.05 NA water immersion objective (Olympus, Tokyo Japan). An emission filter collecting light between 380 nm-420 nm (Chroma, Bellow Falls VT) were used to image DAPI, while the fluorescence emission of Alexa 568 was collected using a filter between 565 nm and 635 nm (Chroma, Bellow Falls VT).

### Macro Fluorescence Imaging

Images were taken on a Nikon AZ100M “Macro” laser scanning confocal configured with long working distance low magnification lenses. The microscope is equipped with the standard 405 nm, 488 nm, 561 nm, and 640 nm laser lines and has PMT detectors with a detection range from 400-700 nm. To reduce signal drop-off at the image edges we used an optical zoom factor of 2.1× and increased our lateral resolution using a digital zoom factor of 1.873×.

### Flow Cytometry

For preparation of samples for flow cytometry analysis, cells were initially dissociated into single cell suspensions by accutase treatment (Stemcell Technologies, 07920, 30-60 min, 37° C., 5% CO₂) and then fixed and permeabilized using fixation/permeabilization buffers (Foxp3/Transcription Factor Staining Buffer Set, 00-5523). Cells were stained with primary and secondary antibodies as described above for immunofluorescence. Flow cytometry was conducted using a BD LSRFortessa cell analyzer and data were analyzed using Flowjo™ (FlowJo™ Software Version 8.7). The list of antibodies and working dilutions is provided in Table S3.

### Human Synapsin::Channelrhodopsin2-EYFP Enteric Ganglioids Blue Light Activation

Enteric ganglioids were either exposed to blue light (100% laser intensity, 3×1-min exposure with 30 s intervals, EVOS FL) or left out in ambient light. Enteric ganglioids were then incubated for 45 minutes at 37° C. before dissociation, fixation and permeabilization for flow cytometry (see above). Cells were stained using antibodies against cFos (abcam, ab190289) and TUBB3 (Biolegend, 801202).

### Bulk RNA-Seq Data Analysis

Total RNA was extracted using PureLink™ RNA Mini Kit. First strand cDNA was then synthesized with the Quantseq Forward Library preparation kit from Lexogen. Illumina compatible RNA sequencing libraries were prepared with Quantseq and pooled and sequenced on Illumina Hiseq 4000 platform at the UCSF Center for Advanced Technology. UMIs were extracted from the fastq files with umi_tools, and cutadapt was used to remove short and low-quality reads. The reads were aligned to human GENCODE v.34 reference genome using STAR aligner, and the duplicate reads were collapsed using umi_tools. Gene level counts were measured using HTSeq and compared using DESeq2.

### Single Cell and Single Nuclei RNA Sequencing Sample Preparation and Data Collection

All tubes and pipet tips used for cell harvesting were pre-treated with 1% BSA in 1×PBS. Cells were dissociated in Accutase (Stem Cell) at 37° C., in 10 min increments, with end-to-end rotation, until single cell suspension was obtained. The cells were washed in Cell Staining Buffer (Biolegend) and stained with TotalSeq HTO antibodies for 30 min on ice. The cells were washed twice in Cell Staining Buffer and filtered through a 40 μm pipette tip strainer (BelArt). The cells were counted using Trypan Blue dye and hemocytometer and pooled for sequencing. scRNA-seq libraries were prepared with Chromium Next GEM Single Cell 3′ Kit v3.1 (10× Genomics), with custom amplification of TotalSeq HTO sequences (Biolegend). The libraries were sequenced on Illumina NovaSeq sequencer in the Center for Advanced Technologies (UCSF). The cell feature matrices were extracted using kallisto/bustools, and demultiplexed using seurat.

### Quality Control and Cell Filtration

Datasets were analyzed in R v4.0.3 with Seurat v4 (Hao et al., 2021). The number of reads mapping to mitochondrial and ribosomal gene transcripts per cell were calculated using the “PercentageFeatureSet” function. Cells were identified as poor quality and subsequently removed independently for each dataset based on the number of unique features captured per cell, the number of UMI captured per cell and the percentage of reads mapping to mitochondrial transcripts per cell. Dataset specific quality control metric cutoffs can be found in Table S4.

TABLE S4

| | nFeatures | nFeatures | nCounts | percent |
|---|---|---|---|---|
| | greater | less | less | mitochondrial |
| Dataset | than | than | than | reads less than |
| stage 1 control BR1 | 1250 | 7000 | 40000 | 10% |
| stage 1 control BR2 | 1250 | 7000 | 40000 | 10% |
| stage 1 pp121 BR1 | 1250 | 7000 | 40000 | 10% |
| stage 1 pp121 BR2 | 1250 | 7000 | 40000 | 10% |
| stage 2 control BR1 | 1250 | 6500 | 40000 | 10% |
| stage 2 control BR2 | 1250 | 6500 | 40000 | 10% |
| Stage 2 control 2D | | | | |
| D10 | 1250 | 3500 | 20000 | 5% |
| D15 | 1500 | 3500 | 20000 | 10% |
| Morarach et al. | | | | |
| E15 | 1000 | 6000 | 40000 | 5% |
| E18 | 1000 | 8000 | 40000 | 10% |
| P21 | 1500 | 8000 | 60000 | 25% (if |
| | | | | nfeatures > |
| | | | | 4000), 10% (if |
| | | | | nfeatures < 4000) |
| Drokhlyansky et al. | | | | |
| Human | 1000 | NA | NA | NA |
| Mouse | 1000 | NA | NA | NA |

### Dimensionality Reduction, Clustering and Annotation

Where applicable, biological replicate samples were first merged using the base R “merge” function. Counts matrices were log normalized with a scaling factor of 10,000 and 2,000 variable features were identified using the “vst” method. For datasets specified in Table S5, count matrices of biological replicate samples were integrated using Seurat integration functions with default parameters. Cell cycle phase was predicted using the “CellCycleScoring” function with Seurat's S and G2M features provided in “cc.genes.” The variable feature sets were scaled and centered, and the following variables were regressed out: nFeatures, nCounts, mitochondrial gene percentage, ribosomal gene percentage, S score and G2M score. Principal Components Analysis (PCA) was run using default settings and Uniform Manifold Approximation and Projection (UMAP) dimensionality reduction was performed using the PCA reduction. The shared nearest neighbors (SNN) graph was computed using default settings and cell clustering was performed using the default Louvain algorithm. Quality control metrics were visualized per cluster to identify and remove clusters of low-quality cells (less than average nFeatures or nCounts and higher than average mitochondrial and ribosomal gene percentage) (Table S5). The above pipeline was performed again on datasets after the removal of any low-quality cell clusters and for the sub-clustering analysis of the enteric neural crest, enteric neurons, nitrergic neurons and enteric glia. The number of principal components used for UMAP reduction and SNN calculation was determined by principal component standard deviation and varied for each dataset. The number of principal components used for SNN and UMAP calculation and the resolution used for clustering of each dataset can be found in Table S5. Cluster markers were found using the Wilcoxon Rank Sum test and clusters were annotated based on the expression of known cell type marker genes (Table S6). Following cell type annotation, gene dropout values were imputed using adaptively-thresholded low rank approximation (ALRA) (Linderman et al., 2018). The rank-k approximation was automatically chosen for each dataset and all other parameters were set as the default values. The imputed gene expression is shown in all plots and used in all downstream analysis unless otherwise specified.

TABLE S5

| | | | # PCs Used | UMAP | UMAP | | UMAP | | Cluster(s) removed |
|---|---|---|---|---|---|---|---|---|---|
| | Cell Type | Batch Correction | (SNN and | min. | n. | UMAP | umap. | Clustering | and re-clustered? |
| Dataset | Subset | Method | UMAP) | dist | neighbors | metric | method | Resolution | (Reason) |
| Stage 1 Control | All Cells | NA (regular merge) | 18 | 0.3 | 30 | cosine | uwot | 0.4 | Yes (High ribosomal |
| | | | | | | | | | gene percentage) |
| Stage 1 Control | Neurons | NA (regular merge) | 17 | 0.3 | 30 | cosine | uwot | 0.6 | Yes (High ribosomal |
| | | | | | | | | | gene percentage) |
| Stage 1 Control | Nitrergic | NA (regular merge) | 14 | 0.3 | 30 | cosine | uwot | 0.3 | |
| | Neurons | | | | | | | | |
| Stage 1 Control and | All Cells | NA (regular merge) | 18 | 0.3 | 30 | cosine | uwot | 0.2 | |
| Stage 1 Control and | Neurons | NA (regular merge) | 16 | 0.3 | 30 | cosine | uwot | 0.6 | Yes (High ribosomal |
| PP121 | | | | | | | | | gene percentage) |
| Stage 1 Control and | Nitrergic | NA (regular merge) | 16 | 0.3 | 30 | cosine | uwot | 0.3 | |
| PP121 | Neurons | | | | | | | | |
| Stage 2 Control | All Cells | NA (regular merge) | 24 | 0.3 | 30 | cosine | uwot | 0.2 | |
| Stage 2 Control | Neurons | NA (regular merge) | 20 | 0.3 | 30 | cosine | uwot | 20 | |
| Stage 2 Control | Glia | NA (regular merge) | 25 | 0.3 | 30 | cosine | uwot | 0.2 | |
| Stage 2 Control 2D | All Cells | Seurat Integration | 19 | 0.3 | 30 | cosine | uwot | 0.1 | |
| Stage 2 Control 2D | Neurons | Seurat Integration | 20 | 0.3 | 30 | cosine | uwot | 0.3 | |
| Stage 2 Control 2D | Glia | Seurat Integration | 20 | 0.3 | 30 | cosine | uwot | 0.2 | |
| D10 | All Cells | NA | 11 | 0.3 | 30 | cosine | uwot | 0.1 | |
| D10 | ENCs | NA | 17 | 0.3 | 30 | cosine | uwot | 0.2 | Yes (High expression |
| | | | | | | | | | of placode genes) |
| D15 | All Cells | NA | 16 | 0.3 | 30 | cosine | uwot | 0.1 | |
| D15 | ENCs | NA | 21 | 0.3 | 30 | cosine | uwot | 0.5 | Yes (High SYP |
| Morarach et al. | | | | | | | | | |
| E15 | All Cells | | 30 | 0.48 | 69 | correlation | uwot-learn | 0.9 | Yes (Not enteric) |
| E18 | All Cells | | 30 | 0.5 | 49 | correlation | uwot-learn | 0.9 | Yes (Not enteric) |
| E18 | Progenitors | | 30 | 0.5 | 30 | correlation | uwot-learn | 0.2 | |
| | and Glia | | | | | | | | |
| P21 | All Cells | | 35 | 0.5 | 30 | correlation | uwot-learn | 1 | Yes (low |
| P21 | Glia | | 30 | 0.5 | 30 | correlation | uwot-learn | 0.2 | |
| Drokhlyansky et al. | | | | | | | | | |
| Human | All Cells | MNN | 30 | 0.3 | 30 | cosine | uwot | 0.1 | |
| Human | Neurons | MNN | 30 | 0.3 | 30 | cosine | uwot | 0.1 | |
| Human | Nitrergic | MNN | 30 | 0.3 | 30 | cosine | uwot | 0.6 | |
| | Neurons | | | | | | | | |
| Human | Glia | MNN | 30 | 0.3 | 30 | cosine | uwot | 0.3 | |
| Mouse | Neurons | MNN | 30 | 0.3 | 30 | cosine | uwot | 0.1 | |
| Mouse | Glia | MNN | 30 | 0.3 | 30 | cosine | uwot | 0.3 | |

TABLE S6

| Enteric | | | | | | Smooth |
|---|---|---|---|---|---|---|
| Neurons | Progenitor | Epithelial | Mesenchymal | Glia | RPE | Muscle |
| NRXN3 | SOX2 | CDH1 | PRRX1 | GFAP | MITF | ACTA1 |
| NRXN1 | NOTCH1 | EPCAM | RUNX2 | ERBB4 | OTX2 | PAX7 |
| DCX | MEF2C | KRT19 | TWIST1 | NTRK2 | PAX6 | MYOD1 |
| MAPT | PAX7 | | COL11A1 | NTRK3 | SIX3 | MYL4 |
| ELAVL2 | | | COL1A2 | PAX3 | OTX1 | CHRNA1 |
| NRCAM | | | COL1A1 | EDNRB | TYR | TNNT2 |
| RBFOX3 | | | COL3A1 | FZD3 | TRPM1 | MYOG |
| NCAM1 | | | COL5A2 | SOX2 | DCT | DES |
| NRG1 | | | FN1 | VEGFC | TYRP1 | TBX1 |
| SYN1 | | | LAMA4 | | | |
| SYP | | | EDNRA | | | |
| | | | PDGFRA | | | |
| | | | PDGFRB | | | |

### Analysis of Published Datasets

Quality control. Criteria used by the original authors of each dataset was used to identify and remove poor quality cells. Dataset specific quality control metric cutoffs can be found in Table S4.

Dimensionality Reduction and Clustering. Datasets were analyzed with Seurat using the methods and parameters described by the original authors.

Morarach et al.: For all datasets, count matrices were normalized, mitochondrial gene percentage was regressed and 3000 variable features were returned using the “SCTransform” function. Highly expressed sex-specific and immediate early genes (Xist, Gm13305, Tsix, Eif253y, Ddx3y, Uty, Fos, Jun, Junb, Egr1) were removed form the variable feature list prior to running PCA. The dataset specific parameters used for the “RunUMAP”, “FindNeighbors” and “FindClusters” functions can be found in Table S5. Cell annotations determined by the authors were used for cell types and neuronal subtypes.

Drokhlyansky et al.: For all datasets, count matrices were log normalized with a scaling factor of 10,000 and 2,000 variable features were identified using the “vst” method. Batch correction by “Unique ID” was performed using mutual nearest neighbors correction (MNN) with the “RunFastMNN” Seurat Wrappers function. The dataset specific parameters used for the “RunUMAP”, “FindNeighbors” and “FindClusters” functions can be found in Table S5 Cell annotations determined by the authors were used for cell types and neuronal subtypes. For consistency of comparison, gene dropout values were imputed using ALRA for all published datasets using automatically determined rank-k approximations and all other default values. The imputed gene expression is shown in all plots and used in all downstream analysis unless otherwise specified.

Glia Sub-clustering analysis. Glia were sub-clustered using methods similar to the original analysis pipeline described by each author above.

Morarach et al.: The E18 dataset contained a single transcriptionally homogenous glia cluster, so the glia and progenitor populations were sub-clustered together to provide comparative cell populations needed for downstream analysis. Subset datasets were then normalized, mitochondrial gene percentage was regressed and 3000 variable features were returned using the “SCTransform” function. Highly expressed sex-specific and immediate early genes (Xist, Gm13305, Tsix, Eif253y, Ddx3y, Uty, Fos, Jun, Junb, Egr1) were removed from the variable feature list prior to running PCA. The dataset specific parameters used for the “RunUMAP”, “FindNeighbors” and “FindClusters” functions can be found in Table S5.

Drokhlyansky et al.: Glia subset datasets were log normalized with a scaling factor of 10,000 and 2,000 variable features were identified using the “vst” method. Batch correction by “Unique ID” was performed using mutual nearest neighbors correction (MNN) with the “RunFastMNN” Seurat Wrappers function. The dataset specific parameters used for the “RunUMAP”, “FindNeighbors” and “FindClusters” functions can be found in Table S5.

### Gene Group Expression Characterization

Gene lists were compiled for genes belonging to ten different functional groups (transcription factors, neurotransmitter synthesis, neuropeptides, neurotransmitter receptors, neuropeptide receptors, cytokines, cytokine receptors, secreted signaling ligands, ligand receptors, and surface markers) (Table S7). For each dataset, the gene lists were filtered to remove low abundance genes (detected in less than 25% of cells of each cluster). Genes from these lists were determined to be exclusively expressed by a cluster if greater than 25% of cells of only a single cluster expressed the gene.

TABLE S7

| ETFs | Neurotransmitter | Neuropeptides | NT Receptors | Neuropeptide | Cytokine | Cytokine | Secreted | Receptors | Surface |
|---|---|---|---|---|---|---|---|---|---|
| SRY | CHAT | ADCYAP1 | CHRM1 | ADCYAP1R1 | IL1A | CXCR1 | BDNF | EPHA1 | ABCB1 |
| SOX1 | SLC18A3 | ADIPOQ | CHRM2 | ADIPOR1 | IL1B | CXCR2 | NGF | EPHA2 | ABCG2 |
| SOX2 | ACHE | ADM | CHRM3 | ADIPOR2 | IL1F10 | IL1RAP | NTF3 | EPHA3 | ACE |
| SOX3 | SLC5A7 | ADM2 | CHRM4 | AGTR1 | IL2 | IL1RAPL1 | NTF4 | EPHA4 | ACKR1 |
| SOX4 | NOS1 | AGRP | CHRM5 | AGTR2 | IL3 | IL1RAPL2 | NTF6A | EPHA5 | ADAM10 |
| SOX5 | NOS1AP | AGT | CHRNA1 | APLNR | IL4 | IL1RL1 | NTF6B | EPHA6 | ADAM17 |
| SOX6 | ARG1 | APLN | CHRNA2 | AVPR1A | IL5 | IL1RL2 | NTF6G | EPHA7 | ADAM8 |
| SOX7 | ARG2 | AVP | CHRNA3 | AVPR1B | IL6 | IL1RN | ARTN | EPHA8 | ADGRE2 |
| SOX8 | ASL | CALCA | CHRNA4 | CALCR | IL7 | IL1R1 | GDNF | EPHA10 | ADGRE5 |
| SOX9 | ASS1 | CALCB | CHRNA5 | CALCRL | CXCL8 | IL1R2 | NRTN | EPHB1 | ALCAM |
| SOX10 | TH | CARTPT | CHRNA6 | CCKAR | IL9 | IL2RA | PSPN | EPHB2 | ALK |
| SOX11 | PAH | CBLN1 | CHRNA7 | CCKBR | IL10 | IL2RB | WNT1 | EPHB3 | ANPEP |
| SOX12 | DDC | CBLN2 | CHRNA9 | CLR | IL11 | IL2RG | WNT2 | EPHB4 | ART1 |
| SOX13 | TYR | CBLN3 | CHRNA10 | CRHR1 | IL12A | IL3RA | WNT2B | EPHB6 | ART4 |
| SOX14 | SPR | CBLN4 | CHRNB1 | CRHR1 | IL12B | IL4R | WNT3 | NOTCH1 | ATP1B3 |
| SOX15 | QDPR | CCK | CHRNB2 | CRHR2 | IL13 | IL5RA | WNT3A | NOTCH2 | B3GAT1 |
| SOX17 | TAT | CGC | CHRNB3 | EDNRA | IL15 | IL6R | WNT4 | NOTCH3 | BCAM |
| SOX18 | SLC6A3 | CHGA | CHRNB4 | EDNRB | IL16 | IL6ST | WNT5A | NOTCH4 | BMPR1A |
| SOX21 | QDPR | CHGB | CHRND | GALR1 | IL17A | IL7R | WNT5B | TLR1 | BMPR1B |
| SOX30 | SLC18A2 | CORT | CHRNE | GALR2 | IL17B | IL9R | WNT6 | TLR2 | BSG |
| POU1F1 | TPH1 | CRH | CHRNG | GALR3 | IL17C | IL10RA | WNT7A | TLR3 | BST1 |
| POU2F1 | DDC | EDN1 | GUCY1A2 | GCGR | IL17D | IL10RB | WNT7B | TLR4 | BST2 |
| POU2F2 | TDO2 | EDN2 | GUCY1A3 | GHSR | IL17F | IL11RA | WNT8A | TLR5 | BTLA |
| POU2F3 | IDO1 | EDN3 | GUCY1B2 | GHSR | IL18 | IL12RB1 | WNT8B | TLR6 | BTN3A1 |
| POU3F1 | IDO2 | GAL | GUCY1B3 | GIPR | IL19 | IL12RB2 | WNT9A | TLR7 | C5AR1 |
| POU3F2 | SLC6A4 | GALP | HTR1A | GNRHR | IL20 | IL13RA1 | WNT9B | TLR8 | CCR1 |
| POU3F3 | SLC18A2 | GAST | HTR1B | GNRHR2 | IL21 | IL13RA2 | WNT10A | TLR9 | CCR2 |
| POU3F4 | GAD1 | GHRH | HTR1D | GRPR | IL22 | IL15RA | WNT10B | TLR10 | CCR3 |
| POU4F1 | GAD2 | GHRL | HTR1E | HCRTR1 | IL23A | IL17RA | WNT11 | CDH1 | CCR4 |
| POU4F2 | SLC32A1 | GIP | HTR1F | HCRTR2 | IL24 | IL17RB | WNT16 | CDH2 | CCR5 |
| POU4F3 | GLUL | GNRH1 | HTR2A | IGF1R | IL25 | IL17RC | BMP1 | CDH3 | CCR6 |
| POU5F1 | SLC17A6 | GNRH2 | HTR2B | IGF2R | IL26 | IL17RD | BMP2 | CDH4 | CCR7 |
| POU5F1P1 | SLC17A7 | GRP | HTR2C | INSR | IL27 | IL17RE | BMP3 | CDH5 | CCR8 |
| POU5F1P3 | SLC17A8 | HCRT | HTR4 | KISS1R | IL31 | IL18RAP | BMP4 | CDH6 | CCR9 |
| POU5F1P4 | | IAPP | HTR5A | LEPR | IL32 | IL18R1 | BMP5 | CDH7 | CD101 |
| POU5F2 | | IGF1 | HTR5BP | MCHR1 | IL33 | IL20RA | BMP6 | CDH8 | CD109 |
| POU6F1 | | IGF2 | HTR6 | MCHR2 | IL34 | IL20RB | BMP7 | CDH9 | CD14 |
| POU6F2 | | INS | HTR7 | MLNR | IL36A | IL21R | BMP8A | CDH10 | CD151 |
| PAX1 | | KISS1 | HTR3A | NMBR | IL36B | IL22RA1 | BMP8B | CDH11 | CD160 |
| PAX2 | | KNG1 | HTR3B | NMUR1 | IL36G | IL22RA2 | BMP10 | CDH12 | CD163 |
| PAX3 | | LEP | HTR3C | NMUR2 | IL37 | IL27RA | BMP15 | CDH13 | CD164 |
| PAX4 | | MLN | HTR3D | NPBWR1 | CD40LG | IL31RA | FGF1 | CDH15 | CD177 |
| PAX5 | | NAMPT | HTR3E | NPBWR2 | CD70 | CD40 | FGF2 | CDH16 | CD180 |
| PAX6 | | NMB | DRD1 | NPFFR1 | EDA | EDA2R | FGF3 | CDH17 | CD19 |
| PAX7 | | NMS | DRD2 | NPFFR2 | FASLG | EDAR | FGF4 | CDH18 | CD1A |
| PAX8 | | NMU | DRD3 | NPR1 | LTA | FAS | FGF5 | CDH19 | CD1B |
| PAX9 | | NPB | DRD4 | NPR2 | LTB | LTBR | FGF6 | CDH20 | CD1C |
| FOXA1 | | NPFF | DRD5 | NPR3 | TNF | PGLYRP1 | FGF7 | CDH22 | CD1D |
| FOXA2 | | NPPA | GABRA1 | NPSR1 | TNFSF4 | TNFR1 | FGF8 | CDH23 | CD1E |
| FOXA3 | | NPPB | GABRA2 | NPY1R | TNFSF8 | TNFR2 | FGF9 | CDH24 | CD2 |
| FOXB1 | | NPPC | GABRA3 | NPY2R | TNFSF9 | TNFRSF4 | FGF10 | CDH26 | CD200 |
| FOXB2 | | NPS | GABRA4 | NPY4R | TNFSF10 | TNFRSF6B | FGF11 | CDHR1 | CD207 |
| FOXC1 | | NPVF | GABRA5 | NPY4R2 | TNFSF11 | TNFRSF7 | FGF12 | CDHR2 | CD209 |
| FOXC2 | | NPW | GABRA6 | NPY5R | TNFSF12 | TNFRSF8 | FGF13 | CDHR3 | CD22 |
| FOXD1 | | NPY | GABRB1 | NPY6R | TNFSF13 | TNFRSF9 | FGF14 | CDHR4 | CD226 |
| FOXD2 | | NTS | GABRB2 | NRXN1 | TNFSF13B | TNFRSF11A | FGF16 | CDHR5 | CD24 |
| FOXD3 | | NUCB2 | GABRB3 | NRXN2 | TNFSF14 | TNFRSF11B | FGF17 | CELSR1 | CD244 |
| FOXD4 | | NXPH1 | GABRD | NRXN3 | TNFSF15 | TNFRSF12A | FGF18 | CELSR2 | CD247 |
| FOXD5 | | NXPH2 | GABRE | NTSR1 | IFNA1 | TNFRSF13B | FGF19 | CELSR3 | CD248 |
| FOXE1 | | NXPH3 | GABRG1 | NTSR2 | IFNA2 | TNFRSF13C | FGF20 | CLSTN1 | CD27 |
| FOXE2 | | NXPH4 | GABRG2 | OPRD1 | IFNA4 | TNFRSF14 | FGF21 | CLSTN2 | CD274 |
| FOXE3 | | OXT | GABRG3 | OPRK1 | IFNA5 | TNFRSF17 | FGF22 | CLSTN3 | CD276 |
| FOXF1 | | PDYN | GABRP | OPRL1 | IFNA7 | TNFRSF18 | FGF23 | DCHS1 | CD28 |
| FOXF2 | | PENK | GABRQ | OPRM1 | IFNA8 | TNFRSF19 | SEMA3A | DCHS2 | CD300A |
| FOXG1 | | PMCH | GABRR1 | OXTR | IFNA10 | TNFRSF19L | SEMA3B | DSC1 | CD300C |
| FOXH1 | | PNOC | GABRR2 | PRLHR | IFNA13 | TNFRSF21 | SEMA3C | DSC2 | CD300E |
| FOXI1 | | POMC | GABRR3 | PRLR | IFNA14 | TNFRSF25 | SEMA3D | DSC3 | CD302 |
| FOXI2 | | PPY | GABBR1 | RAMP1 | IFNB1 | TRAILR1 | SEMA3E | DSG1 | CD320 |
| FOXJ1 | | PRL | GABBR2 | RAMP2 | IFNE | TRAILR2 | SEMA3F | DSG2 | CD33 |
| FOXJ2 | | PRLH | GRIA1 | RAMP3 | IFNG | TRAILR3 | SEMA3G | DSG3 | CD34 |
| FOXJ3 | | PTHLH | GRIA2 | RXFP1 | IFNW1 | TRAILR4 | SEMA4A | DSG4 | CD36 |
| FOXK1 | | PYY | GRIA3 | RXFP2 | IFNZ | IFNAR1 | SEMA4B | FAT1 | CD37 |
| FOXK2 | | RETLA | GRIA4 | RXFP3 | TGFB1 | IFNAR2 | SEMA4C | FAT2 | CD38 |
| FOXM1 | | RETLB | GRID1 | SCTR | TGFB2 | IFNGR1 | SEMA4D | FAT3 | CD3D |
| FOXN1 | | RETLG | GRID2 | SSTR1 | TGFB3 | IFNGR2 | SEMA4F | FAT4 | CD3E |
| FOXN2 | | RETN | GRIK1 | SSTR2 | CCL1 | ACVR1C | SEMA4G | PCDH1 | CD3G |
| FOXN3 | | RLN1 | GRIK2 | SSTR3 | CCL2 | ATF2 | SEMA5A | PCDH7 | CD4 |
| FOXN4 | | RLN2 | GRIK3 | SSTR4 | CCL3 | ENG | SEMA5B | PCDH8 | CD40 |
| FOXO1 | | RLN3 | GRIK4 | SSTR5 | CCL3L1 | TGFBR1 | SEMA6A | PCDH9 | CD40LG |
| FOXO3 | | SCG2 | GRIK5 | TACR1 | CCL3L3 | TGFBR2 | SEMA6B | PCDH10 | CD44 |
| FOXO4 | | SCG3 | GRIN1 | TACR2 | CCL4 | TGFBR3 | SEMA6C | PCDH11X | CD46 |
| FOXO6 | | SCG5 | GRIN2A | TACR3 | CCL4L1 | ACKR1 | SEMA6D | PCDH11Y | CD47 |
| FOXP1 | | SCT | GRIN2B | TRHR | CCL4L2 | ACKR2 | SEMA7A | PCDH12 | CD48 |
| FOXP2 | | SST | GRIN2C | :UTSR2 | CCL5 | ACKR3 | AMH | PCDH15 | CD5 |
| FOXP3 | | TAC1 | GRIN2D | VIPR1 | CCL7 | ACKR4 | ANGPT1 | PCDH17 | CD52 |
| FOXP4 | | TAC3 | GRIN3A | VIPR2 | CCL8 | CCRL2 | ANGPT2 | PCDH18 | CD53 |
| FOXQ1 | | TRH | GRIN3B | | CCL11 | PITPNM3 | ANGPT4 | PCDH19 | CD55 |
| FOXR1 | | UBL5 | GRM1 | | CCL13 | CCR1 | ANGPTL1 | PCDH20 | CD58 |
| FOXR2 | | UCN | GRM2 | | CCL14 | CCR10 | ANGPTL3 | PCDHA1 | CD59 |
| TBXT | | UCN2 | GRM3 | | CCL15 | CCR2 | ANGPTL7 | PCDHA2 | CD6 |
| TBR1 | | UCN3 | GRM4 | | CCL16 | CCR3 | ANXA1 | PCDHA3 | CD63 |
| TBX1 | | UTS2 | GRM5 | | CCL17 | CCR4 | APELA | PCDHA4 | CD68 |
| TBX2 | | VGF | GRM6 | | CCL18 | CCR5 | APP | PCDHA5 | CD69 |
| TBX3 | | VIP | GRM7 | | CCL19 | CCR6 | AREG | PCDHA6 | CD7 |
| TBX4 | | | GRM8 | | CCL20 | CCR7 | ARF1 | PCDHA7 | CD70 |
| TBX5 | | | | | CCL21 | CCR8 | ARHGDIB | PCDHA8 | CD72 |
| TBX6 | | | | | CCL22 | CCR9 | BID | PCDHA9 | CD74 |
| TBX10 | | | | | CCL23 | CX3CR1 | C3 | PCDHA10 | CD79A |
| TBX15 | | | | | CCL24 | CXCR1 | C5 | PCDHA11 | CD79B |
| TBX18 | | | | | CCL25 | CXCR2 | CALM1 | PCDHA12 | CD80 |
| TBX19 | | | | | CCL26 | CXCR3 | CALM2 | PCDHA13 | CD81 |
| TBX20 | | | | | CCL27 | CXCR4 | CALM3 | PCDHA14 | CD82 |
| TBX21 | | | | | CCL28 | CXCR5 | CAMP | PCDHAC1 | CD83 |
| TBX22 | | | | | CXCL1 | CXCR6 | CD274 | PCDHAC2 | CD84 |
| HOXA1 | | | | | CXCL2 | XCR1 | CFLAR | PCDHACT | CD86 |
| HOXA2 | | | | | CXCL3 | | CGA | PCDHB1 | CD8A |
| HOXA3 | | | | | CXCL5 | | CGB3 | PCDHB2 | CD8B |
| HOXA4 | | | | | CXCL6 | | COPA | PCDHB3 | CD9 |
| HOXA5 | | | | | CXCL8 | | CSF3 | PCDHB4 | CD93 |
| HOXA6 | | | | | CXCL9 | | CTSG | PCDHB5 | CD96 |
| HOXA7 | | | | | CXCL10 | | DEFB4A | PCDHB6 | CD99 |
| HOXA8 | | | | | CXCL11 | | EPO | PCDHB7 | CDCP1 |
| HOXA9 | | | | | CXCL12 | | F2 | PCDHB8 | CDH1 |
| HOXA10 | | | | | CXCL13 | | FGA | PCDHB9 | CDH2 |
| HOXA11 | | | | | CXCL14 | | FGB | PCDHB10 | CDH5 |
| HOXA12 | | | | | CXCL16 | | FGG | PCDHB11 | CEACAM1 |
| HOXA13 | | | | | CXCL17 | | FLT3LG | PCDHB12 | CEACAM3 |
| HOXB1 | | | | | CX3CL1 | | FN1 | PCDHB13 | CEACAM5 |
| HOXB2 | | | | | XCL1 | | FSHB | PCDHB14 | CEACAM6 |
| HOXB3 | | | | | XCL2 | | GCG | PCDHB15 | CEACAM8 |
| HOXB4 | | | | | | | GDF1 | PCDHB16 | CLEC10A |
| HOXB5 | | | | | | | GDF2 | PCDHB17P | CLEC12A |
| HOXB6 | | | | | | | GDF3 | PCDHB18P | CLEC4A |
| HOXB7 | | | | | | | GDF9 | PCDHB19P | CLEC4C |
| HOXB8 | | | | | | | GDF10 | PCDHGA1 | CLEC4D |
| HOXB9 | | | | | | | GUCA2A | PCDHGA2 | CLEC4M |
| HOXB13 | | | | | | | GUCA2B | PCDHGA3 | CLEC7A |
| HOXC4 | | | | | | | HEBP1 | PCDHGA4 | CLEC9A |
| HOXC5 | | | | | | | HTT | PCDHGA5 | CR1 |
| HOXC6 | | | | | | | ICAM1 | PCDHGA6 | CR2 |
| HOXC8 | | | | | | | ICAM2 | PCDHGA7 | CRTAM |
| HOXC9 | | | | | | | ICOSLG | PCDHGA8 | CSF1R |
| HOXC10 | | | | | | | INSL3 | PCDHGA9 | CSF2RA |
| HOXC11 | | | | | | | INSL5 | PCDHGA10 | CSF2RB |
| HOXC12 | | | | | | | LGALS1 | PCDHGA11 | CSF3R |
| HOXC13 | | | | | | | LGALS3 | PCDHGA12 | CTLA4 |
| HOXD1 | | | | | | | LGALS3BP | PCDHGB1 | CXCR1 |
| HOXD3 | | | | | | | LGALS9 | PCDHGB2 | CXCR2 |
| HOXD4 | | | | | | | LHB | PCDHGB3 | CXCR3 |
| HOXD8 | | | | | | | LIF | PCDHGB4 | CXCR4 |
| HOXD9 | | | | | | | LRRC4B | PCDHGB5 | CXCR5 |
| HOXD10 | | | | | | | MMP1 | PCDHGB6 | CXCR6 |
| HOXD11 | | | | | | | MMP13 | PCDHGB7 | DDR1 |
| HOXD12 | | | | | | | MT-RNR2 | PCDHGB8P | DPP4 |
| HOXD13 | | | | | | | NCAN | PCDHGB9P | ENG |
| RUNX1 | | | | | | | NCR3LG1 | PCDHGC3 | ENPEP |
| RUNX2 | | | | | | | NDP | PCDHGC4 | ENPP3 |
| RUNX3 | | | | | | | PDCD1LG2 | PCDHGC5 | ENTPD1 |
| E2F1 | | | | | | | PPBP | PCDHGCT | EPCAM |
| E2F2 | | | | | | | PROC | RET | ERBB2 |
| E2F3 | | | | | | | PROK1 | AATK | EVI2B |
| E2F4 | | | | | | | PROK2 | ACVR1 | F11R |
| E2F5 | | | | | | | PSAP | ACVR1B | F3 |
| E2F6 | | | | | | | PTH | ACVR1C | FAS |
| E2F7 | | | | | | | PTH2 | ACVR2A | FASLG |
| E2F8 | | | | | | | PTN | ACVR2B | FCAMR |
| EHF | | | | | | | QRFP | ACVRL1 | FCAR |
| ELF1 | | | | | | | RAC1 | ALK | FCER2 |
| ELF2 | | | | | | | RAC2 | AMHR2 | FCGR1A |
| ELF3 | | | | | | | RAC3 | AXL | FCGR2A |
| ELF4 | | | | | | | RARRES2 | BMPR1A | FCGR2B |
| ELF5 | | | | | | | RB1 | BMPR1B | FCGR2C |
| ELK1 | | | | | | | RGS2 | BMPR2 | FCGR3A |
| ELK3 | | | | | | | SAA1 | CSF1R | FCGR3B |
| ELK4 | | | | | | | SELPLG | DDR1 | FCRL1 |
| ERF | | | | | | | SPP1 | DDR2 | FCRL2 |
| ERG | | | | | | | THPO | EGFR | FCRL3 |
| ETS1 | | | | | | | TSHB | ERBB2 | FCRL4 |
| ETS2 | | | | | | | UTS2B | ERBB3 | FCRL5 |
| ETV1 | | | | | | | VCAM1 | ERBB4 | FGFR1 |
| ETV2 | | | | | | | VTN | FGFR1 | FGFR2 |
| ETV3 | | | | | | | VWF | FGFR2 | FGFR3 |
| ETV3L | | | | | | | WIF1 | FGFR3 | FGFR4 |
| ETV4 | | | | | | | | FGFR4 | FLT3 |
| ETV5 | | | | | | | | FLT1 | FUT3 |
| ETV6 | | | | | | | | FLT3 | FUT4 |
| ETV7 | | | | | | | | FLT4 | FZD10 |
| FEV | | | | | | | | GUCY2C | FZD4 |
| FLI1 | | | | | | | | GUCY2D | FZD9 |
| GABPA | | | | | | | | GUCY2EP | GGT1 |
| SPDEF | | | | | | | | GUCY2F | GP1BA |
| SPIB | | | | | | | | GUCY2GP | GP1BB |
| SPIC | | | | | | | | IGF1R | GP5 |
| SPI1 | | | | | | | | INSR | GP9 |
| FOS | | | | | | | | INSRR | GYPA |
| FOSB | | | | | | | | KDR | GYPB |
| FOSL1 | | | | | | | | KIT | GYPC |
| FOSL2 | | | | | | | | LMTK2 | HAVCR1 |
| JUN | | | | | | | | LMTK3 | HMMR |
| JUNB | | | | | | | | LTK | ICAM1 |
| JUND | | | | | | | | MERTK | ICAM2 |
| | | | | | | | | MET | ICAM3 |
| | | | | | | | | MST1R | ICAM4 |
| | | | | | | | | MUSK | ICOS |
| | | | | | | | | NPR1 | ICOSLG |
| | | | | | | | | NPR2 | IFITM1 |
| | | | | | | | | NTRK1 | IFNGR1 |
| | | | | | | | | NTRK2 | IGF1R |
| | | | | | | | | NTRK3 | IGF2R |
| | | | | | | | | PDGFRA | IGLL1 |
| | | | | | | | | PDGFRB | IGSF8 |
| | | | | | | | | PTK7 | IL10RA |
| | | | | | | | | RET | IL10RB |
| | | | | | | | | ROR1 | IL12RB1 |
| | | | | | | | | ROR2 | IL13RA1 |
| | | | | | | | | ROS1 | IL13RA2 |
| | | | | | | | | RYK | IL15RA |
| | | | | | | | | STYK1 | IL17RA |
| | | | | | | | | TEK | IL18R1 |
| | | | | | | | | TIE1 | IL18RAP |
| | | | | | | | | TYRO3 | IL1R1 |
| | | | | | | | | MC1R | IL1R2 |
| | | | | | | | | MC2R | IL21R |
| | | | | | | | | MC3R | IL2RA |
| | | | | | | | | MC4R | IL2RB |
| | | | | | | | | MC5R | IL2RG |
| | | | | | | | | MCHR1 | IL3RA |
| | | | | | | | | MCHR2 | IL4R |
| | | | | | | | | | IL5RA |
| | | | | | | | | | IL6R |
| | | | | | | | | | IL6ST |
| | | | | | | | | | IL7R |
| | | | | | | | | | IL9R |
| | | | | | | | | | INSR |
| | | | | | | | | | ITGA1 |
| | | | | | | | | | ITGA2 |
| | | | | | | | | | ITGA2B |
| | | | | | | | | | ITGA3 |
| | | | | | | | | | ITGA4 |
| | | | | | | | | | ITGA5 |
| | | | | | | | | | ITGA6 |
| | | | | | | | | | ITGAD |
| | | | | | | | | | ITGAE |
| | | | | | | | | | ITGAL |
| | | | | | | | | | ITGAM |
| | | | | | | | | | ITGAV |
| | | | | | | | | | ITGAX |
| | | | | | | | | | ITGB1 |
| | | | | | | | | | ITGB2 |
| | | | | | | | | | ITGB3 |
| | | | | | | | | | ITGB4 |
| | | | | | | | | | JAG1 |
| | | | | | | | | | JAM2 |
| | | | | | | | | | KDR |
| | | | | | | | | | KEL |
| | | | | | | | | | KIR2DL1 |
| | | | | | | | | | KIR2DL2 |
| | | | | | | | | | KIR2DL3 |
| | | | | | | | | | KIR2DL4 |
| | | | | | | | | | KIR2DL5A |
| | | | | | | | | | KIR2DS1 |
| | | | | | | | | | KIR2DS2 |
| | | | | | | | | | KIR2DS4 |
| | | | | | | | | | KIR2DS5 |
| | | | | | | | | | KIR3DL1 |
| | | | | | | | | | KIR3DL2 |
| | | | | | | | | | KIR3DL3 |
| | | | | | | | | | KIR3DP1 |
| | | | | | | | | | KIT |
| | | | | | | | | | KLRB1 |
| | | | | | | | | | KLRC1 |
| | | | | | | | | | KLRC2 |
| | | | | | | | | | KLRD1 |
| | | | | | | | | | KLRK1 |
| | | | | | | | | | L1CAM |
| | | | | | | | | | LAG3 |
| | | | | | | | | | LAIR1 |
| | | | | | | | | | LAIR2 |
| | | | | | | | | | LAMP1 |
| | | | | | | | | | LAMP2 |
| | | | | | | | | | LAMP3 |
| | | | | | | | | | LEPR |
| | | | | | | | | | LIFR |
| | | | | | | | | | LILRA1 |
| | | | | | | | | | LILRA2 |
| | | | | | | | | | LILRA3 |
| | | | | | | | | | LILRA4 |
| | | | | | | | | | LILRA5 |
| | | | | | | | | | LILRA6 |
| | | | | | | | | | LILRB1 |
| | | | | | | | | | LILRB2 |
| | | | | | | | | | LILRB3 |
| | | | | | | | | | LILRB4 |
| | | | | | | | | | LILRB5 |
| | | | | | | | | | LILRP1 |
| | | | | | | | | | LILRP2 |
| | | | | | | | | | LRP1 |
| | | | | | | | | | LY75 |
| | | | | | | | | | LY9 |
| | | | | | | | | | MCAM |
| | | | | | | | | | MELTF |
| | | | | | | | | | MME |
| | | | | | | | | | MPL |
| | | | | | | | | | MRC1 |
| | | | | | | | | | MRC2 |
| | | | | | | | | | MS4A1 |
| | | | | | | | | | MSR1 |
| | | | | | | | | | MST1R |
| | | | | | | | | | MUC1 |
| | | | | | | | | | NCAM1 |
| | | | | | | | | | NCR1 |
| | | | | | | | | | NCR2 |
| | | | | | | | | | NCR3 |
| | | | | | | | | | NECTIN1 |
| | | | | | | | | | NECTIN2 |
| | | | | | | | | | NECTIN3 |
| | | | | | | | | | NGFR |
| | | | | | | | | | NRP1 |
| | | | | | | | | | NT5E |
| | | | | | | | | | PDCD1 |
| | | | | | | | | | PDCD1LG2 |
| | | | | | | | | | PDGFRA |
| | | | | | | | | | PDGFRB |
| | | | | | | | | | PECAM1 |
| | | | | | | | | | PI16 |
| | | | | | | | | | PLAUR |
| | | | | | | | | | PLXNC1 |
| | | | | | | | | | PRNP |
| | | | | | | | | | PROCR |
| | | | | | | | | | PROM1 |
| | | | | | | | | | PSG1 |
| | | | | | | | | | PTGDR2 |
| | | | | | | | | | PTGFRN |
| | | | | | | | | | PTPRC |
| | | | | | | | | | PTPRJ |
| | | | | | | | | | PVR |
| | | | | | | | | | RHAG |
| | | | | | | | | | RHCE |
| | | | | | | | | | RHD |
| | | | | | | | | | S1PR1 |
| | | | | | | | | | SDC1 |
| | | | | | | | | | SDC2 |
| | | | | | | | | | SELE |
| | | | | | | | | | SELL |
| | | | | | | | | | SELP |
| | | | | | | | | | SELPLG |
| | | | | | | | | | SEMA4D |
| | | | | | | | | | SEMA7A |
| | | | | | | | | | SIGLEC1 |
| | | | | | | | | | SIGLEC5 |
| | | | | | | | | | SIGLEC6 |
| | | | | | | | | | SIGLEC7 |
| | | | | | | | | | SIGLEC9 |
| | | | | | | | | | SIRPA |
| | | | | | | | | | SIRPB1 |
| | | | | | | | | | SIRPG |
| | | | | | | | | | SLAMF1 |
| | | | | | | | | | SLAMF6 |
| | | | | | | | | | SLAMF7 |
| | | | | | | | | | SLAMF8 |
| | | | | | | | | | SLC44A1 |
| | | | | | | | | | SLC4A1 |
| | | | | | | | | | SLC7A5 |
| | | | | | | | | | SPN |
| | | | | | | | | | TEK |
| | | | | | | | | | TFRC |
| | | | | | | | | | THBD |
| | | | | | | | | | THY1 |
| | | | | | | | | | TLR1 |
| | | | | | | | | | TLR10 |
| | | | | | | | | | TLR2 |
| | | | | | | | | | TLR3 |
| | | | | | | | | | TLR4 |
| | | | | | | | | | TLR6 |
| | | | | | | | | | TLR8 |
| | | | | | | | | | TLR9 |
| | | | | | | | | | TNFRSF10A |
| | | | | | | | | | TNFRSF10B |
| | | | | | | | | | TNFRSF10C |
| | | | | | | | | | TNFRSF10D |
| | | | | | | | | | TNFRSF11A |
| | | | | | | | | | TNFRSF12A |
| | | | | | | | | | TNFRSF13B |
| | | | | | | | | | TNFRSF13C |
| | | | | | | | | | TNFRSF14 |
| | | | | | | | | | TNFRSF17 |
| | | | | | | | | | TNFRSF18 |
| | | | | | | | | | TNFRSF1A |
| | | | | | | | | | TNFRSF1B |
| | | | | | | | | | TNFRSF21 |
| | | | | | | | | | TNFRSF4 |
| | | | | | | | | | TNFRSF8 |
| | | | | | | | | | TNFRSF9 |
| | | | | | | | | | TNFSF10 |
| | | | | | | | | | TNFSF11 |
| | | | | | | | | | TNFSF13 |
| | | | | | | | | | TNFSF13B |
| | | | | | | | | | TNFSF14 |
| | | | | | | | | | TNFSF4 |
| | | | | | | | | | TNFSF8 |
| | | | | | | | | | TREM1 |
| | | | | | | | | | TSPAN7 |
| | | | | | | | | | VCAM1 |
| | | | | | | | | | VPREB1 |

### Cell Type Transcriptional Signature Scoring

To find transcriptionally similar cell populations between two datasets, first the differentially expressed (DE) genes of the reference dataset are calculated from the non-imputed gene counts with the “FindAllMarkers” function using the Wilcoxon Rank Sum test and only genes with a positive fold change were returned. The DE gene lists are first filtered to remove genes not present in the query dataset. Then for each cell cluster in the reference dataset, a transcriptional signature gene list is made from the top 100 DE genes sorted by increasing adjusted p-value. The query dataset is then scored for the transcriptional signature gene lists of each reference dataset cell cluster using the “AddModuleScore” function based on the query dataset's imputed gene counts.

### Spearman Correlations

The transcriptional correlation of cell clusters in two datasets was calculated from the non-imputed gene counts and utilized Seurat's integration functions to first find 3,000 anchor features based on the first 30 dimensions of the canonical correlation analysis and then integrate the two datasets using the same number of dimensions. The expression of these 3000 anchor features was then scaled and centered in the merged data object and the average scaled expression of each anchor feature was calculated for each dataset's cell clusters of interest using the “AverageExpression” function. A Spearman correlation matrix comparing all cell clusters to all cell clusters was generated based on the average scaled expression of the 3000 anchor features.

### SWNE Projections

The reference and query dataset counts matrices are first filtered to only include genes detected in both datasets. Similarly Weighted Nonnegative Embeddings (SWNE) are then generated for the reference dataset using the SWNE v0.6 package. First, nonnegative matrix factorization (NFM) generates component factors from the 3000 variable features calculated from the reference dataset non-imputed gene counts. Two dimensional component factor embeddings are calculated using sammon mapping and the cells and specified key genes are embedded in 2D relative to the component factors. Finally, a SNN network is calculated from the reference dataset and is used to smooth the cell positions. The query dataset is then mapped onto the reference dataset's 2D component factor space by first projecting the query dataset onto the reference dataset's NFM factors. The resulting query dataset cell embeddings are then smoothed by projection onto the reference dataset's SNN network.

### Myenteric and Submucosal Scoring

Patient metadata published by the authors was used to separately group neurons or glia by tissue layer origin. Pan-neuronal and pan-glial myenteric and submucosal gene signatures were created by performing the Wilcoxon Rank Sum test to identify DE genes between the myenteric and submucosal cell groupings. Neuronal and glial datasets were scored with the cell-type specific tissue layer signatures by first ordering the gene lists by increasing adjusted p-value and removing genes not detected in the dataset to be scored. The “AddModuleScore” function was then used to score the cells for the 100 most significantly enriched genes for each tissue layer.

### Neurochemical Identification of Neurons

The neurochemical identification of neurons was performed independently for each neurotransmitter to accommodate multi-neurochemical identities. For each neurotransmitter, a core set of genes were selected consisting of the rate-limiting synthesis enzyme(s), metabolism enzymes and transport proteins (Table S1). Cells were first scored for each neurotransmission associated gene set using the “AddModuleScore” function. A cell was then annotated as “x-ergic” if the cell's expression of a rate limiting enzyme was greater than 0 and the cell's module score for the corresponding gene set was greater than 0. A cell was annotated as “Other” if both criteria were not met. Multi-neurochemical identities were determined by concatenating the individually determined single neurochemical identities of each cell. The overall prevalence of each neurochemical identity per dataset was calculated by summing the total number of cells annotated for each single identity and calculating the percentage of each “x-ergic” identity from this sum total.

### Neurotransmitter Response Scoring

Separate gene lists were created containing all receptors activated by each neurotransmitter. Cells were scored for their expression of each neurotransmitter receptor family gene set using the “AddModuleScore” function.

### Glia GSEA Hierarchical Clustering

For each sub-clustered glia dataset, DE genes for each glial subtype were calculated using the “FindAllMarkers” function. Gene set enrichment analysis (GSEA) for the MSigDB gene ontology sets was performed on each glia subytpe's upregulated DE genes (positive log 2 fold change only) sorted by decreasing log 2 fold change using fgsea v1.16. Normalized Enrichment Scores (NES) were calculated for gene sets containing a minimum of 15 genes in the DE gene list with the scoreType set to “positive”. Each glial subtype's GSEA results were filtered to only include biological process gene sets but not filtered based on significance as to not limit the result to pathways enriched in the highest fold change genes. The NES of the filtered gsea results for all glial subtypes were then merged and pathways not detected in a glial subtype were assigned a NES of 0. Hierarchical clustering was then performed based on the NESs to cluster both the gene ontology pathways and the glial subtypes. After glia classes were determined by clustering, pathways enriched in each class were identified by filtering for pathways with an NES greater than 1.1 in all subtypes of a given class.

### PP121 vs Control Gene Expression Correlation

To compare the gene expression of control and PP121 treated cell types, neuronal subtypes and NO neuron subtypes, a subset dataset of each cell type and subtype annotation was first created. For each subset, the non-imputed average expression of all genes was then calculated for the control and PP121 treated cells using the “AverageExpression” function and natural log transformed for plotting. R² values comparing the control and PP121 natural log expression values were calculated from linear modeling using the “y ˜x” formula. cFOS expression screening

Stage 2 enteric ganglioids were dissociated using accutase and single cell suspensions (in ENC medium) were distributed in wells of V-bottom 96-well plates. Compounds from a neuronal signaling compound library (Selleckchem, USA) were added at 1 μM using a pin tool and cells were incubated for 75 minutes at 37° C. Afterwards, cells were washed with PBS, and were immediately fixed for flow cytometry.

### NO Release Assay

For high-throughput measures of nitric oxide (NO) release, stage 1 2D ENS cultures (96-well plates) were used. After washing cells with Tyrode's solution [NaCl (129 mM), KCl (5 mM), CaCl₂) (2 mM), MgCl (1 mM), glucose (30 mM) and HEPES (25 mM) at pH 7.4], 70 μl/well of Tyrode's solution was added to each well. Neuronal signaling compounds (Selleckchem, USA) were added at 1 μM using a pin tool. After a 45 minutes incubation at 37° C., supernatants were used to determine NO release using an NO assay kit (Invitrogen, EMSNO). Briefly, the kit uses the enzyme nitrate reductase that converts nitrate to nitrite which is then detected as a colored azo dye absorbing light at 540 nm. NO release for each compound was presented as the A540 nm relative to the vehicle (DMSO).

High-Throughput Screening to Identify Compounds that Enrich NO Neurons

Day 15 H9 hESC-derived enteric crestospheres were dissociated into single cells (accutase, Stemcell Technologies, 07920, 30 min, 37° C.), resuspended in ENC medium and were transferred into 384-well plates. Plates were incubated for 2 hours for cells to attach. Using a pin tool, drugs from a library of 1694 inhibitors (SelleckChem, USA) were added to wells at the final concentration of 1 μM and plates were incubated with drugs until D20, when media were changed to ENC with no drugs. At day 40, cells were fixed, stained for NOS1 and imaged using InCellAnalyzer 2000 (GE Healthcare, USA). Hits were selected based on the fold increase of the percentage of NOS1⁺ cells relative to the wells treated with vehicle (DMSO).

### Surface Marker Screening

For human surface marker screening, PP121-treated NOS1::GFP enteric ganglioids from four independent differentiations were pooled, dissociated into single cells (accutase, Stemcell Technologies, 07920, 30-60 min, 37° C., 5% CO₂) and fixed (Foxp3/Transcription Factor Staining Buffer Set, 00-5523, 30 min, 4° C.). Cells were permeabilized and blocked (same staining kit) prior to incubation with anti GFP antibody (abcam, ab13970, 4° C.). After three washes, cells were stained with Alexa Fluor 488-conjugated secondary antibody (40 min, RT). Secondary antibody solution was removed (3×washes) and cells were incubated with a blocking buffer containing PBS and 2% FBS (30 min, on ice). Cells were divided in a 240:16 ratio corresponding to the number of library antibodies raised in mouse and rat, and received anti-mouse and anti-rat Alexa Fluor 647-conjugated secondary antibodies respectively. Then, they were distributed into V-bottom 96-well plates and treated with library antibodies for 30 minutes on ice (BD Biosciences, 560747). After two washes, surface marker and GFP signals were quantified by high-throughput flow cytometry (BD LSRFortessa). NO neuron specific surface markers were identified based on the highest sensitivity (highest percentage of CD⁺GFP⁺ cells) and highest specificity (lowest percentage of CD⁺GFP⁻ cells).

### Drug Target Interaction Prediction

We obtained canonical SMILES of our hits from PubChem (De Giorgio et al., 2016; Niesler et al., 2021) and generated a list of their known and predicted targets by combining data from the following databases: BindingDB (https://www.bindingdb.org/).
Carlsbad (http://carlsbad.health,unm.edu/), DINIES (https://www.genome.jp/tools/dinies/), PubChem BioAssay (https://pubchem.ncbi.nlm.nih.gov/, filtered for active interactions), SEA (http://sea.bkslab.org/, filtered for MaxTC>0.4), SuperDRUG2 (http://cheminfo.charite.de/superdrug2/) and SwissTargetPrediction (http://www.swisstargetprediction.ch/).

### In Vivo Cell Transplantation

Specified pathogen free (SPF) homozygote neuronal nitric oxide synthase knockout mice (B6.129S4-Nos1^{tm1plh}/J; nNos1^{-/-}) were bred and maintained, in individually ventilated cages (IVC), for use as recipients. Animals used for these studies were maintained, and the experiments performed, in accordance with the UK Animals (Scientific Procedures) Act 1986 and approved by the University College London Biological Services Ethical Review Process. Animal husbandry at UCL Biological Services was in accordance with the UK Home Office Certificate of Designation. As Nos1^{-/-} mice are immunocompetent, cyclosporin A (250 jig/ml in drinking water) was administered orally two days prior to transplantation to reduce the possible rejection of donor human cells. Cyclosporin A-treated Nos1^{-/-} mice were chosen at random, from within littermate groups, and stage 1 enteric ganglioids were transplanted into the of P23-P27 mice, via laparotomy under isoflurane anesthetic. Briefly, the distal colon was exposed and enteric ganglioids, containing 0.5-1 M cells were subsequently transplanted to the serosal surface of the distal colon, by mouth pipette, using a pulled glass micropipette. Each transplanted tissue typically received 3 ganglioids which were manipulated on the surface of the distal colon, with the bevel of a 30G needle, to ensure appropriate positioning. Transplanted Nos1^{-/-} mice were maintained with continued free access to cyclosporin A (250 jig/ml) treated drinking water for up to 8 weeks post-transplantation, to ensure extended immunosuppression, before sacrifice and removal of the colon for analysis. As cyclosporin A can affect several signaling pathways and induce gene expression changes, it is crucial to verify immunofluorescence results using appropriate controls such as tissue from cyclosporin A treated untransplanted animals in follow up studies. In addition, other immunocompromised backgrounds (e.g. NSG) will be important to further verify these engraftment results.

### Tissue Preparation and Fixation

Following the excision, the entire colon was pinned in a Sylgard (Dow, MI, USA) lined petri dish and opened along the mesenteric border. The mucosa was subsequently removed by sharp dissection and tissues were fixed in 4% PFA in PBS (45 min-1 hour, 22° C.) for further processing.

### Tissue Staining

Colonic longitudinal muscle myenteric plexus (LMMP) tissues were fixed with 4% PFA (1 h on ice), Thermo scientific, J19943-K2) and blocked and permeabilized with a buffer containing 1% BSA and 1% triton X-100 (in PBS, 45 min, RT). Then, tissues were incubated with primary antibody solutions (in the same buffer, overnight, 4° C.) and were washed three times before treatment with fluorophore-conjugated secondary antibodies (1 h, RT). Samples were stained with DAPI and washed prior to mounting using vectashield (Vector Laboratories, H-1400). Antibodies are listed in Table S3.

### Multi-Electrode Array (MEA) Analysis

Data acquisition: Neuron activity was recorded with the Axion Maestro Edge on Cytoview MEA 24-well plates in 1-hour recording sessions for each condition. Neuromodulator or vehicle were added by removing the plate from the Maestro Edge, half-changing the media with 2× concentrated neuromodulator or vehicle in pre-warmed media, and immediately returning the plate to the Axion to resume recording. Optogenetic stimulation was performed with the Axion Lumos attachment, stimulating all wells of the plate with 488 nm light at 50% intensity, 1 second on, 4 seconds off, 30 times.

Data processing: Raw data were first spike sorted with a modified version of SpikeInterface (https://github.com/SpikeInterface) using MountainSort to identify high quality units by manually scoring based on amplitude, waveform shape, firing rate, and inter-spike interval contamination. For pharmacology experiments, neurons were matched between vehicle and neuromodulator recordings by examining all detected units on a specific electrode after spike scoring and identifying units with identical waveforms. Firing rates of these “paired” units from all wells that received the treatment were compared across the control and neuromodulator conditions. Positive responders were units that had a firing rate change greater than +0.1 Hz; negative responders had a firing rate change less than −0.1 Hz; neutral responders had a firing rate change between −0.1 to +0.1 Hz. For optogenetic experiments, individual units were again extracted with SpikeInterface and manually scored. Recordings were separated into “on” times when the LED was active and “off” times when it was not. All units were compiled and firing rates for each unit were compared during the on and off windows.

### Ex Vivo Colonic Motility Assays

Preparation of solutions: Krebs buffer [NaCl (117 mM), KCl (4.7 mM), NaH₂PO₄ (1.2 mM), MgCl₂ (1.5 mM), CaCl₂·2H₂O (2.5 mM), NaHCO₃ (25 mM), Glucose (11 mM), pH 7.4] was placed in a 37° C. water bath and aerated with 95% O₂ and 5% CO₂ (carbogen) gas mixture for at least 30 minutes prior to experiment onset. “Drug” treatment solutions were freshly prepared by adding the drug compound into Krebs buffer before starting data acquisition. The solution with NOS1 inhibitor was prepared by adding N omega-nitro-L-arginine methyl ester hydrochloride (L-NAME) to the drug solutions making “Drug+L-NAME”.

Tissue dissection: For each experimental replicate, a pair of 8-week-old wild type C57BL6 mice (male) were placed in a sealed chamber and euthanized using CO₂ asphyxiation followed by cervical dislocation. The lower GI tract (cecum and colon) was removed and immediately transferred to 37° C. carbogenated Krebs buffer, with the fecal matter still inside. Adipose tissue and mesentery were removed before placing the colons in the organ bath reservoir of gastrointestinal motility monitor (GIMM) apparatus. GIMM had two reservoirs making simultaneous acquisition of control, and drug-treated colons possible.

Experimental set-up and procedure: GIMM was designed based on a previously reported model (Swaminathan et al., 2016). The organ reservoir of GIMM has two-chambers for recording two specimens simultaneously. It is connected to working solutions kept at 37° C. via a 4-channel peristaltic pump (WPI, PERIPRO-4LS). Lower GI tract was harvested and transferred to the organ bath with the Krebs buffer was flowing through. The cecum was pinned down at the proximal tip and the distal end of the colon was pinned through the serosa/mesentery. Five 10-min (for the first experiments) or sequential 6-min (for the sequential drug treatment in the presence and absence of L-NAME) videos were recorded using the IC capture software (Imaging Source) with a high resolution monochromatic firewire industrial camera (Imaging Source®, DMK41AF02) connected to a ⅔″ 16 mm f/1.4 C-Mount Fixed Focal Lens (Fujinon HF16SA1). While tissue in the control chamber was only exposed to Krebs solution, the order of solutions in the experimental chamber was: Krebs, drug compound, Krebs (6 min each), L-NAME (2 min), L-NAME in the presence of a drug compound (6 min) and Krebs (6 min). The chambers were cleaned after each acquisition.

Data and statistical analysis: VolumetryG9a was used to generate the spatiotemporal map (STM) of each acquisition (Spear et al., 2018). Slow waves (SW) and colonic migrating motor complexes (CMMC) data were generated from STMs. Statistical analyses were performed using PRISM.

### Generating Figure Schematics

We used Adobe Illustrator (version 25.4.1) to generate schematics for the figures. Table 2 is a list of those biomarkers specific for one or a plurality of cells disclosed in the application. Most of these biomarkers are expressed as proteins on the surface of the cells. In some embodiments, the biomarkers are expressed as mRNA within the cells. The biomarkers of FIGS. 8-20, including 8, are disclosed in FIGS. 8E and 8F and matched with the cell type disclosed in those panels. It is understood that, if the cell type is matched with the gene name, then that cell type comprises a protein or expresses the gene that is disclosed. In some embodiments, the cell types disclosed in FIGS. 8-20 express mRNA associated with the accession number in Table 2 or an mRNA that comprises at least about 70%, 75%, 80%, 85%, 90%, 91%, 92%, 93%, 94%, 95%, 96%, 97%, 98%, 99% or 100% sequence identity to the mRNA identified by the accession number of Table 2. In some embodiments, the cell types disclosed in the FIGS. 8-20 express protein associated with the accession number in Table 2 or a protein that comprises at least about 70%, 75%, 80%, 85%, 90%, 91%, 92%, 93%, 94%, 95%, 96%, 97%, 98%, 99% or 100% sequence identity to the protein identified by the accession number of Table 2. The sequences associated with the accession numbers in Table 2 are incorporated by reference in their entireties. The sequences associated with the accession numbers in Table S7 are incorporated by reference in their entireties.

TABLE 2

| biomarker | Genbank Acesssion number | Species | gene name |
|---|---|---|---|
| STMN2 | DQ895968 | Homo sapiens | stathmin 2(STMN2) |
| STMN2 | AK308557 | Homo sapiens | stathmin 2(STMN2) |
| STMN2 | AK297485 | Homo sapiens | stathmin 2(STMN2) |
| STMN2 | AK297594 | Homo sapiens | stathmin 2(STMN2) |
| STMN2 | AL110174 | Homo sapiens | stathmin 2(STMN2) |
| STMN2 | DQ892719 | Homo sapiens | stathmin 2(STMN2) |
| STMN2 | NM_007029 | Homo sapiens | stathmin 2(STMN2) |
| STMN2 | S82024 | Homo sapiens | stathmin 2(STMN2) |
| STMN2 | XM_005251142 | Homo sapiens | stathmin 2(STMN2) |
| STMN2 | BT020034 | Homo sapiens | stathmin 2(STMN2) |
| STMN2 | AI096716 | Homo sapiens | stathmin 2(STMN2) |
| STMN2 | NM_001199214 | Homo sapiens | stathmin 2(STMN2) |
| STMN2 | BQ069488 | Homo sapiens | stathmin 2(STMN2) |
| STMN2 | DC319376 | Homo sapiens | stathmin 2(STMN2) |
| STMN2 | BC006302 | Homo sapiens | stathmin 2(STMN2) |
| STMN2 | AK292737 | Homo sapiens | stathmin 2(STMN2) |
| STMN2 | AK309130 | Homo sapiens | stathmin 2(STMN2) |
| STMN2 | D50375 | Homo sapiens | stathmin 2(STMN2) |
| STMN2 | AK091336 | Homo sapiens | stathmin 2(STMN2) |
| STMN2 | AK299500 | Homo sapiens | stathmin 2(STMN2) |
| STMN2 | CR456833 | Homo sapiens | stathmin 2(STMN2) |
| AQP4 | AB209156 | Homo sapiens | aquaporin 4(AQP4) |
| AQP4 | BI917845 | Homo sapiens | aquaporin 4(AQP4) |
| AQP4 | AB128929 | Homo sapiens | aquaporin 4(AQP4) |
| AQP4 | CD105892 | Homo sapiens | aquaporin 4(AQP4) |
| AQP4 | BC022286 | Homo sapiens | aquaporin 4(AQP4) |
| AQP4 | BI667387 | Homo sapiens | aquaporin 4(AQP4) |
| AQP4 | AK026728 | Homo sapiens | aquaporin 4(AQP4) |
| AQP4 | BC030745 | Homo sapiens | aquaporin 4(AQP4) |
| AQP4 | AV725241 | Homo sapiens | aquaporin 4(AQP4) |
| AQP4 | R35726 | Homo sapiens | aquaporin 4(AQP4) |
| AQP4 | N50070 | Homo sapiens | aquaporin 4(AQP4) |
| AQP4 | AK222684 | Homo sapiens | aquaporin 4(AQP4) |
| AQP4 | XM_011525942 | Homo sapiens | aquaporin 4(AQP4) |
| AQP4 | AK295069 | Homo sapiens | aquaporin 4(AQP4) |
| AQP4 | CK001094 | Homo sapiens | aquaporin 4(AQP4) |
| AQP4 | AL138136 | Homo sapiens | aquaporin 4(AQP4) |
| AQP4 | KF055862 | Homo sapiens | aquaporin 4(AQP4) |
| AQP4 | NM_001650 | Homo sapiens | aquaporin 4(AQP4) |
| AQP4 | NM_004028 | Homo sapiens | aquaporin 4(AQP4) |
| AQP4 | BI596912 | Homo sapiens | aquaporin 4(AQP4) |
| AQP4 | D63412 | Homo sapiens | aquaporin 4(AQP4) |
| AQP4 | U34845 | Homo sapiens | aquaporin 4(AQP4) |
| AQP4 | BC045780 | Homo sapiens | aquaporin 4(AQP4) |
| AQP4 | HQ447648 | Homo sapiens | aquaporin 4(AQP4) |
| AQP4 | HQ901095 | Homo sapiens | aquaporin 4(AQP4) |
| AQP4 | NM_001317387 | Homo sapiens | aquaporin 4(AQP4) |
| AQP4 | U63622 | Homo sapiens | aquaporin 4(AQP4) |
| AQP4 | U63623 | Homo sapiens | aquaporin 4(AQP4) |
| AQP4 | AL119338 | Homo sapiens | aquaporin 4(AQP4) |
| AQP4 | NM_001317384 | Homo sapiens | aquaporin 4(AQP4) |
| AQP4 | BQ638704 | Homo sapiens | aquaporin 4(AQP4) |
| AQP4 | BU687682 | Homo sapiens | aquaporin 4(AQP4) |
| STMN4 | AK309207 | Homo sapiens | stathmin 4(STMN4) |
| STMN4 | AK294023 | Homo sapiens | stathmin 4(STMN4) |
| STMN4 | AL136568 | Homo sapiens | stathmin 4(STMN4) |
| STMN4 | BC011520 | Homo sapiens | stathmin 4(STMN4) |
| STMN4 | AI638208 | Homo sapiens | stathmin 4(STMN4) |
| STMN4 | DQ894219 | Homo sapiens | stathmin 4(STMN4) |
| STMN4 | AK225142 | Homo sapiens | stathmin 4(STMN4) |
| STMN4 | NM_030795 | Homo sapiens | stathmin 4(STMN4) |
| STMN4 | XM_005273652 | Homo sapiens | stathmin 4(STMN4) |
| STMN4 | BC111001 | Homo sapiens | stathmin 4(STMN4) |
| STMN4 | XM_005273655 | Homo sapiens | stathmin 4(STMN4) |
| STMN4 | AK295329 | Homo sapiens | stathmin 4(STMN4) |
| STMN4 | AJ303455 | Homo sapiens | stathmin 4(STMN4) |
| STMN4 | NM_001283054 | Homo sapiens | stathmin 4(STMN4) |
| STMN4 | DQ891040 | Homo sapiens | stathmin 4(STMN4) |
| STMN4 | NM_001283053 | Homo sapiens | stathmin 4(STMN4) |
| STMN4 | AK297415 | Homo sapiens | stathmin 4(STMN4) |
| STMN4 | NM_001283055 | Homo sapiens | stathmin 4(STMN4) |
| SOX2 | BM722297 | Homo sapiens | SRY-box 2(SOX2) |
| SOX2 | KM822781 | Homo sapiens | SRY-box 2(SOX2) |
| SOX2 | AW016610 | Homo sapiens | SRY-box 2(SOX2) |
| SOX2 | BC013923 | Homo sapiens | SRY-box 2(SOX2) |
| SOX2 | L07335 | Homo sapiens | SRY-box 2(SOX2) |
| SOX2 | BM668019 | Homo sapiens | SRY-box 2(SOX2) |
| SOX2 | NM_003106 | Homo sapiens | SRY-box 2(SOX2) |
| SOX2 | Z31560 | Homo sapiens | SRY-box 2(SOX2) |
| SOX2 | AK312595 | Homo sapiens | SRY-box 2(SOX2) |
| SOX2 | BF305585 | Homo sapiens | SRY-box 2(SOX2) |
| SOX2 | CN430685 | Homo sapiens | SRY-box 2(SOX2) |
| SOX2 | DA388996 | Homo sapiens | SRY-box 2(SOX2) |
| SOX2 | EU446654 | Homo sapiens | SRY-box 2(SOX2) |
| SOX2 | AW163619 | Homo sapiens | SRY-box 2(SOX2) |
| CDH6 | XM_017008910 | Homo sapiens | cadherin 6(CDH6) |
| CDH6 | AK291290 | Homo sapiens | cadherin 6(CDH6) |
| CDH6 | XM_017008911 | Homo sapiens | cadherin 6(CDH6) |
| CDH6 | DC353791 | Homo sapiens | cadherin 6(CDH6) |
| CDH6 | AL049227 | Homo sapiens | cadherin 6(CDH6) |
| CDH6 | XR_001741972 | Homo sapiens | cadherin 6(CDH6) |
| CDH6 | BC013907 | Homo sapiens | cadherin 6(CDH6) |
| CDH6 | NM_004932 | Homo sapiens | cadherin 6(CDH6) |
| CDH6 | AK024238 | Homo sapiens | cadherin 6(CDH6) |
| CDH6 | XM_011513921 | Homo sapiens | cadherin 6(CDH6) |
| CDH6 | AU130185 | Homo sapiens | cadherin 6(CDH6) |
| CDH6 | BC000019 | Homo sapiens | cadherin 6(CDH6) |
| CDH6 | D31784 | Homo sapiens | cadherin 6(CDH6) |
| EDNRA | AK304451 | Homo sapiens | endothelin receptor type A(EDNRA) |
| EDNRA | S81539 | Homo sapiens | endothelin receptor type A(EDNRA) |
| EDNRA | DA183901 | Homo sapiens | endothelin receptor type A(EDNRA) |
| EDNRA | BQ006584 | Homo sapiens | endothelin receptor type A(EDNRA) |
| EDNRA | NM_001166055 | Homo sapiens | endothelin receptor type A(EDNRA) |
| EDNRA | L06622 | Homo sapiens | endothelin receptor type A(EDNRA) |
| EDNRA | NM_001256283 | Homo sapiens | endothelin receptor type A(EDNRA) |
| EDNRA | DA956937 | Homo sapiens | endothelin receptor type A(EDNRA) |
| EDNRA | S45956 | Homo sapiens | endothelin receptor type A(EDNRA) |
| EDNRA | S67127 | Homo sapiens | endothelin receptor type A(EDNRA) |
| EDNRA | S57498 | Homo sapiens | endothelin receptor type A(EDNRA) |
| EDNRA | X61950 | Homo sapiens | endothelin receptor type A(EDNRA) |
| EDNRA | AK312812 | Homo sapiens | endothelin receptor type A(EDNRA) |
| EDNRA | NM_001957 | Homo sapiens | endothelin receptor type A(EDNRA) |
| EDNRA | DQ892329 | Homo sapiens | endothelin receptor type A(EDNRA) |
| EDNRA | S81545 | Homo sapiens | endothelin receptor type A(EDNRA) |
| EDNRA | BC022511 | Homo sapiens | endothelin receptor type A(EDNRA) |
| EDNRA | DA775295 | Homo sapiens | endothelin receptor type A(EDNRA) |
| EDNRA | S81542 | Homo sapiens | endothelin receptor type A(EDNRA) |
| EDNRA | NR_045958 | Homo sapiens | endothelin receptor type A(EDNRA) |
| EDNRA | AY275462 | Homo sapiens | endothelin receptor type A(EDNRA) |
| EDNRA | CD723797 | Homo sapiens | endothelin receptor type A(EDNRA) |
| EDNRA | AK315931 | Homo sapiens | endothelin receptor type A(EDNRA) |
| EDNRA | DQ895532 | Homo sapiens | endothelin receptor type A(EDNRA) |
| EDNRA | AF014826 | Homo sapiens | endothelin receptor type A(EDNRA) |
| EDNRA | S63938 | Homo sapiens | endothelin receptor type A(EDNRA) |
| EDNRA | D90348 | Homo sapiens | endothelin receptor type A(EDNRA) |
| EDNRB | S75587 | Homo sapiens | endothelin receptor type B(EDNRB) |
| EDNRB | NM_000115 | Homo sapiens | endothelin receptor type B(EDNRB) |
| EDNRB | NM_001122659 | Homo sapiens | endothelin receptor type B(EDNRB) |
| EDNRB | AF114164 | Homo sapiens | endothelin receptor type B(EDNRB) |
| EDNRB | AF114165 | Homo sapiens | endothelin receptor type B(EDNRB) |
| EDNRB | M74921 | Homo sapiens | endothelin receptor type B(EDNRB) |
| EDNRB | L06623 | Homo sapiens | endothelin receptor type B(EDNRB) |
| EDNRB | AB209198 | Homo sapiens | endothelin receptor type B(EDNRB) |
| EDNRB | AY275463 | Homo sapiens | endothelin receptor type B(EDNRB) |
| EDNRB | S44866 | Homo sapiens | endothelin receptor type B(EDNRB) |
| EDNRB | BM557607 | Homo sapiens | endothelin receptor type B(EDNRB) |
| EDNRB | NR_047024 | Homo sapiens | endothelin receptor type B(EDNRB) |
| EDNRB | AF114163 | Homo sapiens | endothelin receptor type B(EDNRB) |
| EDNRB | BG436360 | Homo sapiens | endothelin receptor type B(EDNRB) |
| EDNRB | NM_003991 | Homo sapiens | endothelin receptor type B(EDNRB) |
| EDNRB | D90402 | Homo sapiens | endothelin receptor type B(EDNRB) |
| EDNRB | NM_001201397 | Homo sapiens | endothelin receptor type B(EDNRB) |
| EDNRB | AK290699 | Homo sapiens | endothelin receptor type B(EDNRB) |
| EDNRB | X99250 | Homo sapiens | endothelin receptor type B(EDNRB) |
| EDNRB | S57283 | Homo sapiens | endothelin receptor type B(EDNRB) |
| EDNRB | BC014472 | Homo sapiens | endothelin receptor type B(EDNRB) |
| EDNRB | H28710 | Homo sapiens | endothelin receptor type B(EDNRB) |
| CDH1 | XM_011523488 | Homo sapiens | cadherin 1(CDH1) |
| CDH1 | BC013851 | Homo sapiens | cadherin 1(CDH1) |
| CDH1 | X12790 | Homo sapiens | cadherin 1(CDH1) |
| CDH1 | XM_011523489 | Homo sapiens | cadherin 1(CDH1) |
| CDH1 | BC146662 | Homo sapiens | cadherin 1(CDH1) |
| CDH1 | EU709494 | Homo sapiens | cadherin 1(CDH1) |
| CDH1 | Z13009 | Homo sapiens | cadherin 1(CDH1) |
| CDH1 | AB025106 | Homo sapiens | cadherin 1(CDH1) |
| CDH1 | AB025105 | Homo sapiens | cadherin 1(CDH1) |
| CDH1 | AI890107 | Homo sapiens | cadherin 1(CDH1) |
| CDH1 | AK309703 | Homo sapiens | cadherin 1(CDH1) |
| CDH1 | L08599 | Homo sapiens | cadherin 1(CDH1) |
| CDH1 | BC141838 | Homo sapiens | cadherin 1(CDH1) |
| CDH1 | NM_004360 | Homo sapiens | cadherin 1(CDH1) |
| CDH1 | X52279 | Homo sapiens | cadherin 1(CDH1) |
| CDH1 | AK311198 | Homo sapiens | cadherin 1(CDH1) |
| CDH1 | AK312551 | Homo sapiens | cadherin 1(CDH1) |
| CDH1 | NM_001317184 | Homo sapiens | cadherin 1(CDH1) |
| CDH1 | AK290012 | Homo sapiens | cadherin 1(CDH1) |
| CDH1 | Z18923 | Homo sapiens | cadherin 1(CDH1) |
| CDH1 | AK297913 | Homo sapiens | cadherin 1(CDH1) |
| CDH1 | NM_001317186 | Homo sapiens | cadherin 1(CDH1) |
| CDH1 | NM_001317185 | Homo sapiens | cadherin 1(CDH1) |
| CDH1 | BC144283 | Homo sapiens | cadherin 1(CDH1) |
| MPZ | BC006491 | Homo sapiens | myelin protein zero(MPZ) |
| MPZ | CD172418 | Homo sapiens | myelin protein zero(MPZ) |
| MPZ | D10537 | Homo sapiens | myelin protein zero(MPZ) |
| MPZ | NM_000530 | Homo sapiens | myelin protein zero(MPZ) |
| MPZ | BM663255 | Homo sapiens | myelin protein zero(MPZ) |
| MPZ | XM_017001321 | Homo sapiens | myelin protein zero(MPZ) |
| MPZ | BT006765 | Homo sapiens | myelin protein zero(MPZ) |
| MPZ | CD515400 | Homo sapiens | myelin protein zero(MPZ) |
| MPZ | DB210759 | Homo sapiens | myelin protein zero(MPZ) |
| MPZ | CD515605 | Homo sapiens | myelin protein zero(MPZ) |
| MPZ | EU176378 | Homo sapiens | myelin protein zero(MPZ) |
| MPZ | BF509916 | Homo sapiens | myelin protein zero(MPZ) |
| MPZ | DQ895885 | Homo sapiens | myelin protein zero(MPZ) |
| MPZ | NM_001315491 | Homo sapiens | myelin protein zero(MPZ) |
| MPZ | AK313555 | Homo sapiens | myelin protein zero(MPZ) |
| MPZ | S66705 | Homo sapiens | myelin protein zero(MPZ) |
| NRCAM | BX538010 | Homo sapiens | neuronal cell adhesion molecule(NRCAM) |
| NRCAM | XM_017012239 | Homo sapiens | neuronal cell adhesion molecule(NRCAM) |
| NRCAM | XM_017012238 | Homo sapiens | neuronal cell adhesion molecule(NRCAM) |
| NRCAM | XM_017012237 | Homo sapiens | neuronal cell adhesion molecule(NRCAM) |
| NRCAM | XM_017012236 | Homo sapiens | neuronal cell adhesion molecule(NRCAM) |
| NRCAM | XM_005250385 | Homo sapiens | neuronal cell adhesion molecule(NRCAM) |
| NRCAM | XM_005250383 | Homo sapiens | neuronal cell adhesion molecule(NRCAM) |
| NRCAM | BC114570 | Homo sapiens | neuronal cell adhesion molecule(NRCAM) |
| NRCAM | BC115736 | Homo sapiens | neuronal cell adhesion molecule(NRCAM) |
| NRCAM | AK299870 | Homo sapiens | neuronal cell adhesion molecule(NRCAM) |
| NRCAM | BX954399 | Homo sapiens | neuronal cell adhesion molecule(NRCAM) |
| NRCAM | XM_011516256 | Homo sapiens | neuronal cell adhesion molecule(NRCAM) |
| NRCAM | XM_011516255 | Homo sapiens | neuronal cell adhesion molecule(NRCAM) |
| NRCAM | XM_005250373 | Homo sapiens | neuronal cell adhesion molecule(NRCAM) |
| NRCAM | XM_011516253 | Homo sapiens | neuronal cell adhesion molecule(NRCAM) |
| NRCAM | DA398450 | Homo sapiens | neuronal cell adhesion molecule(NRCAM) |
| NRCAM | NM_001037133 | Homo sapiens | neuronal cell adhesion molecule(NRCAM) |
| NRCAM | AJ001057 | Homo sapiens | neuronal cell adhesion molecule(NRCAM) |
| NRCAM | NM_001037132 | Homo sapiens | neuronal cell adhesion molecule(NRCAM) |
| NRCAM | AJ001054 | Homo sapiens | neuronal cell adhesion molecule(NRCAM) |
| NRCAM | XM_017012259 | Homo sapiens | neuronal cell adhesion molecule(NRCAM) |
| NRCAM | XM_017012258 | Homo sapiens | neuronal cell adhesion molecule(NRCAM) |
| NRCAM | BU735065 | Homo sapiens | neuronal cell adhesion molecule(NRCAM) |
| NRCAM | XM_011516267 | Homo sapiens | neuronal cell adhesion molecule(NRCAM) |
| NRCAM | AY528240 | Homo sapiens | neuronal cell adhesion molecule(NRCAM) |
| NRCAM | XM_011516266 | Homo sapiens | neuronal cell adhesion molecule(NRCAM) |
| NRCAM | XM_011516265 | Homo sapiens | neuronal cell adhesion molecule(NRCAM) |
| NRCAM | XM_011516262 | Homo sapiens | neuronal cell adhesion molecule(NRCAM) |
| NRCAM | XM_011516261 | Homo sapiens | neuronal cell adhesion molecule(NRCAM) |
| NRCAM | NM_005010 | Homo sapiens | neuronal cell adhesion molecule(NRCAM) |
| NRCAM | AK127035 | Homo sapiens | neuronal cell adhesion molecule(NRCAM) |
| NRCAM | AK092330 | Homo sapiens | neuronal cell adhesion molecule(NRCAM) |
| NRCAM | NM_001193583 | Homo sapiens | neuronal cell adhesion molecule(NRCAM) |
| NRCAM | NM_001193584 | Homo sapiens | neuronal cell adhesion molecule(NRCAM) |
| NRCAM | XM_011516259 | Homo sapiens | neuronal cell adhesion molecule(NRCAM) |
| NRCAM | XM_011516258 | Homo sapiens | neuronal cell adhesion molecule(NRCAM) |
| NRCAM | NM_001193582 | Homo sapiens | neuronal cell adhesion molecule(NRCAM) |
| NRCAM | XM_011516257 | Homo sapiens | neuronal cell adhesion molecule(NRCAM) |
| NRCAM | XM_006716007 | Homo sapiens | neuronal cell adhesion molecule(NRCAM) |
| NRCAM | XM_017012253 | Homo sapiens | neuronal cell adhesion molecule(NRCAM) |
| NRCAM | XM_017012252 | Homo sapiens | neuronal cell adhesion molecule(NRCAM) |
| NRCAM | XM_017012251 | Homo sapiens | neuronal cell adhesion molecule(NRCAM) |
| NRCAM | XM_017012250 | Homo sapiens | neuronal cell adhesion molecule(NRCAM) |
| NRCAM | XM_006716003 | Homo sapiens | neuronal cell adhesion molecule(NRCAM) |
| NRCAM | XM_017012257 | Homo sapiens | neuronal cell adhesion molecule(NRCAM) |
| NRCAM | XM_017012256 | Homo sapiens | neuronal cell adhesion molecule(NRCAM) |
| NRCAM | XM_017012255 | Homo sapiens | neuronal cell adhesion molecule(NRCAM) |
| NRCAM | XM_017012254 | Homo sapiens | neuronal cell adhesion molecule(NRCAM) |
| NRCAM | XM_011516270 | Homo sapiens | neuronal cell adhesion molecule(NRCAM) |
| NRCAM | XM_017012249 | Homo sapiens | neuronal cell adhesion molecule(NRCAM) |
| NRCAM | XM_017012248 | Homo sapiens | neuronal cell adhesion molecule(NRCAM) |
| NRCAM | XM_017012247 | Homo sapiens | neuronal cell adhesion molecule(NRCAM) |
| NRCAM | AK294195 | Homo sapiens | neuronal cell adhesion molecule(NRCAM) |
| NRCAM | XM_011516271 | Homo sapiens | neuronal cell adhesion molecule(NRCAM) |
| NRCAM | AI031622 | Homo sapiens | neuronal cell adhesion molecule(NRCAM) |
| NRCAM | BC098401 | Homo sapiens | neuronal cell adhesion molecule(NRCAM) |
| NRCAM | AB002341 | Homo sapiens | neuronal cell adhesion molecule(NRCAM) |
| NRCAM | DA292156 | Homo sapiens | neuronal cell adhesion molecule(NRCAM) |
| NRCAM | XM_011516269 | Homo sapiens | neuronal cell adhesion molecule(NRCAM) |
| NRCAM | XM_011516268 | Homo sapiens | neuronal cell adhesion molecule(NRCAM) |
| NRCAM | XM_017012242 | Homo sapiens | neuronal cell adhesion molecule(NRCAM) |
| NRCAM | XM_017012241 | Homo sapiens | neuronal cell adhesion molecule(NRCAM) |
| NRCAM | XM_017012240 | Homo sapiens | neuronal cell adhesion molecule(NRCAM) |
| NRCAM | XM_006716014 | Homo sapiens | neuronal cell adhesion molecule(NRCAM) |
| NRCAM | XM_017012246 | Homo sapiens | neuronal cell adhesion molecule(NRCAM) |
| NRCAM | XM_017012245 | Homo sapiens | neuronal cell adhesion molecule(NRCAM) |
| NRCAM | XM_006716012 | Homo sapiens | neuronal cell adhesion molecule(NRCAM) |
| NRCAM | XM_017012244 | Homo sapiens | neuronal cell adhesion molecule(NRCAM) |
| NRCAM | XM_017012243 | Homo sapiens | neuronal cell adhesion molecule(NRCAM) |
| NCKAP5 | NM_207481 | Homo sapiens | NCK associated protein 5(NCKAP5) |
| NCKAP5 | XM_005263659 | Homo sapiens | NCK associated protein 5(NCKAP5) |
| NCKAP5 | NM_207363 | Homo sapiens | NCK associated protein 5(NCKAP5) |
| NCKAP5 | AK057980 | Homo sapiens | NCK associated protein 5(NCKAP5) |
| NCKAP5 | CA438539 | Homo sapiens | NCK associated protein 5(NCKAP5) |
| NCKAP5 | AB005217 | Homo sapiens | NCK associated protein 5(NCKAP5) |
| NCKAP5 | XM_011511097 | Homo sapiens | NCK associated protein 5(NCKAP5) |
| NCKAP5 | XM_017003980 | Homo sapiens | NCK associated protein 5(NCKAP5) |
| NCKAP5 | XM_011511103 | Homo sapiens | NCK associated protein 5(NCKAP5) |
| NCKAP5 | XM_011511102 | Homo sapiens | NCK associated protein 5(NCKAP5) |
| NCKAP5 | XM_011511105 | Homo sapiens | NCK associated protein 5(NCKAP5) |
| NCKAP5 | XM_011511104 | Homo sapiens | NCK associated protein 5(NCKAP5) |
| NCKAP5 | XM_011511101 | Homo sapiens | NCK associated protein 5(NCKAP5) |
| NCKAP5 | XM_011511100 | Homo sapiens | NCK associated protein 5(NCKAP5) |
| NCKAP5 | BC172401 | Homo sapiens | NCK associated protein 5(NCKAP5) |
| NCKAP5 | AY946008 | Homo sapiens | NCK associated protein 5(NCKAP5) |
| NCKAP5 | AY946007 | Homo sapiens | NCK associated protein 5(NCKAP5) |
| NCKAP5 | AK124659 | Homo sapiens | NCK associated protein 5(NCKAP5) |
| NCKAP5 | XM_017003974 | Homo sapiens | NCK associated protein 5(NCKAP5) |
| NCKAP5 | AK092189 | Homo sapiens | NCK associated protein 5(NCKAP5) |
| NCKAP5 | XM_017003979 | Homo sapiens | NCK associated protein 5(NCKAP5) |
| NCKAP5 | BC110831 | Homo sapiens | NCK associated protein 5(NCKAP5) |
| NCKAP5 | XM_011511099 | Homo sapiens | NCK associated protein 5(NCKAP5) |
| NCKAP5 | XM_017003976 | Homo sapiens | NCK associated protein 5(NCKAP5) |
| NCKAP5 | XM_011511098 | Homo sapiens | NCK associated protein 5(NCKAP5) |
| NCKAP5 | XM_017003975 | Homo sapiens | NCK associated protein 5(NCKAP5) |
| NCKAP5 | XM_017003978 | Homo sapiens | NCK associated protein 5(NCKAP5) |
| NCKAP5 | XM_017003977 | Homo sapiens | NCK associated protein 5(NCKAP5) |
| NCKAP5 | XM_005263660 | Homo sapiens | NCK associated protein 5(NCKAP5) |
| WLS | XM_017002390 | Homo sapiens | wntless Wnt ligand secretion mediator(WLS) |
| WLS | AB097018 | Homo sapiens | wntless Wnt ligand secretion mediator(WLS) |
| WLS | AW137622 | Homo sapiens | wntless Wnt ligand secretion mediator(WLS) |
| WLS | BC007211 | Homo sapiens | wntless Wnt ligand secretion mediator(WLS) |
| WLS | AK074583 | Homo sapiens | wntless Wnt ligand secretion mediator(WLS) |
| WLS | BC137113 | Homo sapiens | wntless Wnt ligand secretion mediator(WLS) |
| WLS | AK026744 | Homo sapiens | wntless Wnt ligand secretion mediator(WLS) |
| WLS | AY359035 | Homo sapiens | wntless Wnt ligand secretion mediator(WLS) |
| WLS | CA438784 | Homo sapiens | wntless Wnt ligand secretion mediator(WLS) |
| WLS | BC137109 | Homo sapiens | wntless Wnt ligand secretion mediator(WLS) |
| WLS | AK074984 | Homo sapiens | wntless Wnt ligand secretion mediator(WLS) |
| WLS | DB478499 | Homo sapiens | wntless Wnt ligand secretion mediator(WLS) |
| WLS | BC110826 | Homo sapiens | wntless Wnt ligand secretion mediator(WLS) |
| WLS | BX648748 | Homo sapiens | wntless Wnt ligand secretion mediator(WLS) |
| WLS | DA753300 | Homo sapiens | wntless Wnt ligand secretion mediator(WLS) |
| WLS | AK309779 | Homo sapiens | wntless Wnt ligand secretion mediator(WLS) |
| WLS | BX537492 | Homo sapiens | wntless Wnt ligand secretion mediator(WLS) |
| WLS | NM_024911 | Homo sapiens | wntless Wnt ligand secretion mediator(WLS) |
| WLS | NM_001002292 | Homo sapiens | wntless Wnt ligand secretion mediator(WLS) |
| WLS | BX538320 | Homo sapiens | wntless Wnt ligand secretion mediator(WLS) |
| WLS | AK301613 | Homo sapiens | wntless Wnt ligand secretion mediator(WLS) |
| WLS | AI217373 | Homo sapiens | wntless Wnt ligand secretion mediator(WLS) |
| WLS | NM_001193334 | Homo sapiens | wntless Wnt ligand secretion mediator(WLS) |
| WLS | DQ323735 | Homo sapiens | wntless Wnt ligand secretion mediator(WLS) |
| WLS | BG701224 | Homo sapiens | wntless Wnt ligand secretion mediator(WLS) |
| WLS | XM_011542191 | Homo sapiens | wntless Wnt ligand secretion mediator(WLS) |
| WLS | XM_011542192 | Homo sapiens | wntless Wnt ligand secretion mediator(WLS) |
| PDGFRB | J03278 | Homo sapiens | platelet derived growth factor receptor beta(PDGFRB) |
| PDGFRB | AK293093 | Homo sapiens | platelet derived growth factor receptor beta(PDGFRB) |
| PDGFRB | NM_002609 | Homo sapiens | platelet derived growth factor receptor beta(PDGFRB) |
| PDGFRB | AB209657 | Homo sapiens | platelet derived growth factor receptor beta(PDGFRB) |
| PDGFRB | EU826595 | Homo sapiens | platelet derived growth factor receptor beta(PDGFRB) |
| PDGFRB | M30493 | Homo sapiens | platelet derived growth factor receptor beta(PDGFRB) |
| PDGFRB | XM_005268464 | Homo sapiens | platelet derived growth factor receptor beta(PDGFRB) |
| PDGFRB | XM_011537658 | Homo sapiens | platelet derived growth factor receptor beta(PDGFRB) |
| PDGFRB | M21616 | Homo sapiens | platelet derived growth factor receptor beta(PDGFRB) |
| PDGFRB | XM_011537659 | Homo sapiens | platelet derived growth factor receptor beta(PDGFRB) |
| PDGFRB | AI346188 | Homo sapiens | platelet derived growth factor receptor beta(PDGFRB) |
| PDGFRB | EU176549 | Homo sapiens | platelet derived growth factor receptor beta(PDGFRB) |
| PDGFRB | BC032224 | Homo sapiens | platelet derived growth factor receptor beta(PDGFRB) |
| PDGFRB | DQ892124 | Homo sapiens | platelet derived growth factor receptor beta(PDGFRB) |
| PDGFRB | CN268096 | Homo sapiens | platelet derived growth factor receptor beta(PDGFRB) |
| PDGFRA | XM_006714041 | Homo sapiens | platelet derived growth factor receptor alpha(PDGFRA) |
| PDGFRA | BC063414 | Homo sapiens | platelet derived growth factor receptor alpha(PDGFRA) |
| PDGFRA | AK308353 | Homo sapiens | platelet derived growth factor receptor alpha(PDGFRA) |
| PDGFRA | XM_005265743 | Homo sapiens | platelet derived growth factor receptor alpha(PDGFRA) |
| PDGFRA | XM_017008282 | Homo sapiens | platelet derived growth factor receptor alpha(PDGFRA) |
| PDGFRA | NM_006206 | Homo sapiens | platelet derived growth factor receptor alpha(PDGFRA) |
| PDGFRA | AA599881 | Homo sapiens | platelet derived growth factor receptor alpha(PDGFRA) |
| PDGFRA | M30494 | Homo sapiens | platelet derived growth factor receptor alpha(PDGFRA) |
| PDGFRA | DA678599 | Homo sapiens | platelet derived growth factor receptor alpha(PDGFRA) |
| PDGFRA | X76079 | Homo sapiens | platelet derived growth factor receptor alpha(PDGFRA) |
| PDGFRA | AV689272 | Homo sapiens | platelet derived growth factor receptor alpha(PDGFRA) |
| PDGFRA | XM_011534385 | Homo sapiens | platelet derived growth factor receptor alpha(PDGFRA) |
| PDGFRA | M22734 | Homo sapiens | platelet derived growth factor receptor alpha(PDGFRA) |
| PDGFRA | XM_006714039 | Homo sapiens | platelet derived growth factor receptor alpha(PDGFRA) |
| PDGFRA | XM_017008281 | Homo sapiens | platelet derived growth factor receptor alpha(PDGFRA) |
| PDGFRA | L25829 | Homo sapiens | platelet derived growth factor receptor alpha(PDGFRA) |
| PDGFRA | XM_017008280 | Homo sapiens | platelet derived growth factor receptor alpha(PDGFRA) |
| PDGFRA | AK316578 | Homo sapiens | platelet derived growth factor receptor alpha(PDGFRA) |
| PDGFRA | M21574 | Homo sapiens | platelet derived growth factor receptor alpha(PDGFRA) |
| PDGFRA | AK311006 | Homo sapiens | platelet derived growth factor receptor alpha(PDGFRA) |
| PDGFRA | AA625689 | Homo sapiens | platelet derived growth factor receptor alpha(PDGFRA) |
| PDGFRA | BC015186 | Homo sapiens | platelet derived growth factor receptor alpha(PDGFRA) |
| MEF2C | EU446634 | Homo sapiens | myocyte enhancer factor 2C(MEF2C) |
| MEF2C | AK307883 | Homo sapiens | myocyte enhancer factor 2C(MEF2C) |
| MEF2C | XM_011543400 | Homo sapiens | myocyte enhancer factor 2C(MEF2C) |
| MEF2C | NM_001308002 | Homo sapiens | myocyte enhancer factor 2C(MEF2C) |
| MEF2C | XM_011543401 | Homo sapiens | myocyte enhancer factor 2C(MEF2C) |
| MEF2C | BC152784 | Homo sapiens | myocyte enhancer factor 2C(MEF2C) |
| MEF2C | EU832832 | Homo sapiens | myocyte enhancer factor 2C(MEF2C) |
| MEF2C | GQ129219 | Homo sapiens | myocyte enhancer factor 2C(MEF2C) |
| MEF2C | XM_017009481 | Homo sapiens | myocyte enhancer factor 2C(MEF2C) |
| MEF2C | AW191949 | Homo sapiens | myocyte enhancer factor 2C(MEF2C) |
| MEF2C | FM163484 | Homo sapiens | myocyte enhancer factor 2C(MEF2C) |
| MEF2C | XM_017009482 | Homo sapiens | myocyte enhancer factor 2C(MEF2C) |
| MEF2C | XM_017009483 | Homo sapiens | myocyte enhancer factor 2C(MEF2C) |
| MEF2C | FM180475 | Homo sapiens | myocyte enhancer factor 2C(MEF2C) |
| MEF2C | BP231922 | Homo sapiens | myocyte enhancer factor 2C(MEF2C) |
| MEF2C | AK312472 | Homo sapiens | myocyte enhancer factor 2C(MEF2C) |
| MEF2C | XM_006714625 | Homo sapiens | myocyte enhancer factor 2C(MEF2C) |
| MEF2C | XM_005248511 | Homo sapiens | myocyte enhancer factor 2C(MEF2C) |
| MEF2C | DA516520 | Homo sapiens | myocyte enhancer factor 2C(MEF2C) |
| MEF2C | BC156603 | Homo sapiens | myocyte enhancer factor 2C(MEF2C) |
| MEF2C | S57212 | Homo sapiens | myocyte enhancer factor 2C(MEF2C) |
| MEF2C | AL833274 | Homo sapiens | myocyte enhancer factor 2C(MEF2C) |
| MEF2C | DC318557 | Homo sapiens | myocyte enhancer factor 2C(MEF2C) |
| MEF2C | DC377336 | Homo sapiens | myocyte enhancer factor 2C(MEF2C) |
| MEF2C | DC377710 | Homo sapiens | myocyte enhancer factor 2C(MEF2C) |
| MEF2C | NM_001193349 | Homo sapiens | myocyte enhancer factor 2C(MEF2C) |
| MEF2C | NM_001193347 | Homo sapiens | myocyte enhancer factor 2C(MEF2C) |
| MEF2C | NM_001193348 | Homo sapiens | myocyte enhancer factor 2C(MEF2C) |
| MEF2C | XM_017009478 | Homo sapiens | myocyte enhancer factor 2C(MEF2C) |
| MEF2C | XM_017009479 | Homo sapiens | myocyte enhancer factor 2C(MEF2C) |
| MEF2C | XM_011543396 | Homo sapiens | myocyte enhancer factor 2C(MEF2C) |
| MEF2C | XM_011543397 | Homo sapiens | myocyte enhancer factor 2C(MEF2C) |
| MEF2C | GQ129221 | Homo sapiens | myocyte enhancer factor 2C(MEF2C) |
| MEF2C | BC026341 | Homo sapiens | myocyte enhancer factor 2C(MEF2C) |
| MEF2C | XM_017009475 | Homo sapiens | myocyte enhancer factor 2C(MEF2C) |
| MEF2C | XM_006714619 | Homo sapiens | myocyte enhancer factor 2C(MEF2C) |
| MEF2C | XM_017009476 | Homo sapiens | myocyte enhancer factor 2C(MEF2C) |
| MEF2C | L08895 | Homo sapiens | myocyte enhancer factor 2C(MEF2C) |
| MEF2C | XM_017009477 | Homo sapiens | myocyte enhancer factor 2C(MEF2C) |
| MEF2C | NM_001193350 | Homo sapiens | myocyte enhancer factor 2C(MEF2C) |
| MEF2C | NM_002397 | Homo sapiens | myocyte enhancer factor 2C(MEF2C) |
| MEF2C | XM_017009480 | Homo sapiens | myocyte enhancer factor 2C(MEF2C) |
| MEF2C | GQ129392 | Homo sapiens | myocyte enhancer factor 2C(MEF2C) |
| MEF2C | GQ129393 | Homo sapiens | myocyte enhancer factor 2C(MEF2C) |
| MEF2C | NM_001131005 | Homo sapiens | myocyte enhancer factor 2C(MEF2C) |
| MEF2C | AL833268 | Homo sapiens | myocyte enhancer factor 2C(MEF2C) |
| MEF2C | DA494302 | Homo sapiens | myocyte enhancer factor 2C(MEF2C) |
| RBFOX3 | NM_001039904 | Homo sapiens | RNA binding protein, fox-1 homolog 3(RBFOX3) |
| RBFOX3 | NM_001082575 | Homo sapiens | RNA binding protein, fox-1 homolog 3(RBFOX3) |
| RBFOX3 | XM_011524365 | Homo sapiens | RNA binding protein, fox-1 homolog 3(RBFOX3) |
| RBFOX3 | AK054893 | Homo sapiens | RNA binding protein, fox-1 homolog 3(RBFOX3) |
| RBFOX3 | XM_011524367 | Homo sapiens | RNA binding protein, fox-1 homolog 3(RBFOX3) |
| RBFOX3 | XM_011524366 | Homo sapiens | RNA binding protein, fox-1 homolog 3(RBFOX3) |
| RBFOX3 | BC140939 | Homo sapiens | RNA binding protein, fox-1 homolog 3(RBFOX3) |
| RBFOX3 | LK938159 | Homo sapiens | RNA binding protein, fox-1 homolog 3(RBFOX3) |
| RBFOX3 | BM714144 | Homo sapiens | RNA binding protein, fox-1 homolog 3(RBFOX3) |
| RBFOX3 | BU741507 | Homo sapiens | RNA binding protein, fox-1 homolog 3(RBFOX3) |
| RBFOX3 | XM_017024208 | Homo sapiens | RNA binding protein, fox-1 homolog 3(RBFOX3) |
| RBFOX3 | AK293617 | Homo sapiens | RNA binding protein, fox-1 homolog 3(RBFOX3) |
| RBFOX3 | XM_017024209 | Homo sapiens | RNA binding protein, fox-1 homolog 3(RBFOX3) |
| RBFOX3 | AK124644 | Homo sapiens | RNA binding protein, fox-1 homolog 3(RBFOX3) |
| RBFOX3 | AK126788 | Homo sapiens | RNA binding protein, fox-1 homolog 3(RBFOX3) |
| RBFOX3 | NM_001025448 | Homo sapiens | RNA binding protein, fox-1 homolog 3(RBFOX3) |
| RBFOX3 | BC093713 | Homo sapiens | RNA binding protein, fox-1 homolog 3(RBFOX3) |
| RBFOX3 | XM_011524359 | Homo sapiens | RNA binding protein, fox-1 homolog 3(RBFOX3) |
| RBFOX3 | BX452143 | Homo sapiens | RNA binding protein, fox-1 homolog 3(RBFOX3) |
| RBFOX3 | XM_017024210 | Homo sapiens | RNA binding protein, fox-1 homolog 3(RBFOX3) |
| RBFOX3 | XM_017024211 | Homo sapiens | RNA binding protein, fox-1 homolog 3(RBFOX3) |
| RBFOX3 | AK293905 | Homo sapiens | RNA binding protein, fox-1 homolog 3(RBFOX3) |
| RBFOX3 | DN990270 | Homo sapiens | RNA binding protein, fox-1 homolog 3(RBFOX3) |
| RBFOX3 | XM_011524360 | Homo sapiens | RNA binding protein, fox-1 homolog 3(RBFOX3) |
| RBFOX3 | AK128131 | Homo sapiens | RNA binding protein, fox-1 homolog 3(RBFOX3) |
| RBFOX3 | XM_011524363 | Homo sapiens | RNA binding protein, fox-1 homolog 3(RBFOX3) |
| RBFOX3 | XM_011524362 | Homo sapiens | RNA binding protein, fox-1 homolog 3(RBFOX3) |
| LIMCH1 | AK299835 | Homo sapiens | LIM and calponin homology domains 1(LIMCH1) |
| LIMCH1 | NM_001330674 | Homo sapiens | LIM and calponin homology domains 1(LIMCH1) |
| LIMCH1 | XM_005248060 | Homo sapiens | LIM and calponin homology domains 1(LIMCH1) |
| LIMCH1 | NM_001330672 | Homo sapiens | LIM and calponin homology domains 1(LIMCH1) |
| LIMCH1 | NM_001330793 | Homo sapiens | LIM and calponin homology domains 1(LIMCH1) |
| LIMCH1 | AM393081 | Homo sapiens | LIM and calponin homology domains 1(LIMCH1) |
| LIMCH1 | NM_001330791 | Homo sapiens | LIM and calponin homology domains 1(LIMCH1) |
| LIMCH1 | NM_001330792 | Homo sapiens | LIM and calponin homology domains 1(LIMCH1) |
| LIMCH1 | NM_001112718 | Homo sapiens | LIM and calponin homology domains 1(LIMCH1) |
| LIMCH1 | NM_001112717 | Homo sapiens | LIM and calponin homology domains 1(LIMCH1) |
| LIMCH1 | AK309846 | Homo sapiens | LIM and calponin homology domains 1(LIMCH1) |
| LIMCH1 | NM_001112719 | Homo sapiens | LIM and calponin homology domains 1(LIMCH1) |
| LIMCH1 | BC029735 | Homo sapiens | LIM and calponin homology domains 1(LIMCH1) |
| LIMCH1 | XM_011513645 | Homo sapiens | LIM and calponin homology domains 1(LIMCH1) |
| LIMCH1 | XM_011513646 | Homo sapiens | LIM and calponin homology domains 1(LIMCH1) |
| LIMCH1 | XM_011513643 | Homo sapiens | LIM and calponin homology domains 1(LIMCH1) |
| LIMCH1 | XM_011513644 | Homo sapiens | LIM and calponin homology domains 1(LIMCH1) |
| LIMCH1 | XM_005248058 | Homo sapiens | LIM and calponin homology domains 1(LIMCH1) |
| LIMCH1 | CR749205 | Homo sapiens | LIM and calponin homology domains 1(LIMCH1) |
| LIMCH1 | XM_011513642 | Homo sapiens | LIM and calponin homology domains 1(LIMCH1) |
| LIMCH1 | XM_006713996 | Homo sapiens | LIM and calponin homology domains 1(LIMCH1) |
| LIMCH1 | AK295836 | Homo sapiens | LIM and calponin homology domains 1(LIMCH1) |
| LIMCH1 | NM_001330790 | Homo sapiens | LIM and calponin homology domains 1(LIMCH1) |
| LIMCH1 | XM_005248057 | Homo sapiens | LIM and calponin homology domains 1(LIMCH1) |
| LIMCH1 | XM_011513649 | Homo sapiens | LIM and calponin homology domains 1(LIMCH1) |
| LIMCH1 | CR936664 | Homo sapiens | LIM and calponin homology domains 1(LIMCH1) |
| LIMCH1 | CR936661 | Homo sapiens | LIM and calponin homology domains 1(LIMCH1) |
| LIMCH1 | XM_011513648 | Homo sapiens | LIM and calponin homology domains 1(LIMCH1) |
| LIMCH1 | NM_001330786 | Homo sapiens | LIM and calponin homology domains 1(LIMCH1) |
| LIMCH1 | AK295784 | Homo sapiens | LIM and calponin homology domains 1(LIMCH1) |
| LIMCH1 | NM_001330787 | Homo sapiens | LIM and calponin homology domains 1(LIMCH1) |
| LIMCH1 | NM_001330784 | Homo sapiens | LIM and calponin homology domains 1(LIMCH1) |
| LIMCH1 | NM_001330982 | Homo sapiens | LIM and calponin homology domains 1(LIMCH1) |
| LIMCH1 | NM_001330983 | Homo sapiens | LIM and calponin homology domains 1(LIMCH1) |
| LIMCH1 | AL117572 | Homo sapiens | LIM and calponin homology domains 1(LIMCH1) |
| LIMCH1 | NM_001330788 | Homo sapiens | LIM and calponin homology domains 1(LIMCH1) |
| LIMCH1 | NM_001330789 | Homo sapiens | LIM and calponin homology domains 1(LIMCH1) |
| LIMCH1 | BC095394 | Homo sapiens | LIM and calponin homology domains 1(LIMCH1) |
| LIMCH1 | XM_011513656 | Homo sapiens | LIM and calponin homology domains 1(LIMCH1) |
| LIMCH1 | AL831962 | Homo sapiens | LIM and calponin homology domains 1(LIMCH1) |
| LIMCH1 | XM_011513657 | Homo sapiens | LIM and calponin homology domains 1(LIMCH1) |
| LIMCH1 | XM_011513654 | Homo sapiens | LIM and calponin homology domains 1(LIMCH1) |
| LIMCH1 | XM_011513655 | Homo sapiens | LIM and calponin homology domains 1(LIMCH1) |
| LIMCH1 | BC053639 | Homo sapiens | LIM and calponin homology domains 1(LIMCH1) |
| LIMCH1 | CR933645 | Homo sapiens | LIM and calponin homology domains 1(LIMCH1) |
| LIMCH1 | DA786971 | Homo sapiens | LIM and calponin homology domains 1(LIMCH1) |
| LIMCH1 | XM_011513653 | Homo sapiens | LIM and calponin homology domains 1(LIMCH1) |
| LIMCH1 | AB029025 | Homo sapiens | LIM and calponin homology domains 1(LIMCH1) |
| LIMCH1 | XM_011513651 | Homo sapiens | LIM and calponin homology domains 1(LIMCH1) |
| LIMCH1 | AK311596 | Homo sapiens | LIM and calponin homology domains 1(LIMCH1) |
| LIMCH1 | XM_005248067 | Homo sapiens | LIM and calponin homology domains 1(LIMCH1) |
| LIMCH1 | AK125004 | Homo sapiens | LIM and calponin homology domains 1(LIMCH1) |
| LIMCH1 | AK294774 | Homo sapiens | LIM and calponin homology domains 1(LIMCH1) |
| LIMCH1 | XM_005248061 | Homo sapiens | LIM and calponin homology domains 1(LIMCH1) |
| LIMCH1 | XM_011513658 | Homo sapiens | LIM and calponin homology domains 1(LIMCH1) |
| LIMCH1 | NM_001289122 | Homo sapiens | LIM and calponin homology domains 1(LIMCH1) |
| LIMCH1 | AK027231 | Homo sapiens | LIM and calponin homology domains 1(LIMCH1) |
| LIMCH1 | AK299297 | Homo sapiens | LIM and calponin homology domains 1(LIMCH1) |
| LIMCH1 | XM_017007901 | Homo sapiens | LIM and calponin homology domains 1(LIMCH1) |
| LIMCH1 | XM_017007900 | Homo sapiens | LIM and calponin homology domains 1(LIMCH1) |
| LIMCH1 | AK302674 | Homo sapiens | LIM and calponin homology domains 1(LIMCH1) |
| LIMCH1 | BC068200 | Homo sapiens | LIM and calponin homology domains 1(LIMCH1) |
| LIMCH1 | CR936601 | Homo sapiens | LIM and calponin homology domains 1(LIMCH1) |
| LIMCH1 | XM_017007905 | Homo sapiens | LIM and calponin homology domains 1(LIMCH1) |
| LIMCH1 | XM_017007904 | Homo sapiens | LIM and calponin homology domains 1(LIMCH1) |
| LIMCH1 | XM_017007903 | Homo sapiens | LIM and calponin homology domains 1(LIMCH1) |
| LIMCH1 | XM_017007902 | Homo sapiens | LIM and calponin homology domains 1(LIMCH1) |
| LIMCH1 | NM_001289124 | Homo sapiens | LIM and calponin homology domains 1(LIMCH1) |
| LIMCH1 | XM_005248072 | Homo sapiens | LIM and calponin homology domains 1(LIMCH1) |
| LIMCH1 | XM_005248074 | Homo sapiens | LIM and calponin homology domains 1(LIMCH1) |
| LIMCH1 | XM_005248075 | Homo sapiens | LIM and calponin homology domains 1(LIMCH1) |
| LIMCH1 | NM_001112720 | Homo sapiens | LIM and calponin homology domains 1(LIMCH1) |
| LIMCH1 | XM_017007899 | Homo sapiens | LIM and calponin homology domains 1(LIMCH1) |
| LIMCH1 | DC325132 | Homo sapiens | LIM and calponin homology domains 1(LIMCH1) |
| LIMCH1 | XM_017007898 | Homo sapiens | LIM and calponin homology domains 1(LIMCH1) |
| LIMCH1 | XM_017007897 | Homo sapiens | LIM and calponin homology domains 1(LIMCH1) |
| LIMCH1 | XM_017007896 | Homo sapiens | LIM and calponin homology domains 1(LIMCH1) |
| LIMCH1 | XM_017007895 | Homo sapiens | LIM and calponin homology domains 1(LIMCH1) |
| LIMCH1 | XM_017007894 | Homo sapiens | LIM and calponin homology domains 1(LIMCH1) |
| LIMCH1 | AK026815 | Homo sapiens | LIM and calponin homology domains 1(LIMCH1) |
| LIMCH1 | BX537916 | Homo sapiens | LIM and calponin homology domains 1(LIMCH1) |
| LIMCH1 | NM_014988 | Homo sapiens | LIM and calponin homology domains 1(LIMCH1) |
| LIMCH1 | CR936610 | Homo sapiens | LIM and calponin homology domains 1(LIMCH1) |
| LIMCH1 | CR936658 | Homo sapiens | LIM and calponin homology domains 1(LIMCH1) |
| LIMCH1 | BC023546 | Homo sapiens | LIM and calponin homology domains 1(LIMCH1) |
| LIMCH1 | BX640692 | Homo sapiens | LIM and calponin homology domains 1(LIMCH1) |
| LIMCH1 | AK298915 | Homo sapiens | LIM and calponin homology domains 1(LIMCH1) |
| LIMCH1 | AM392601 | Homo sapiens | LIM and calponin homology domains 1(LIMCH1) |
| FNDC3B | AK027052 | Homo sapiens | fibronectin type III domain containing 3B(FNDC3B) |
| FNDC3B | NM_022763 | Homo sapiens | fibronectin type III domain containing 3B(FNDC3B) |
| FNDC3B | AF543840 | Homo sapiens | fibronectin type III domain containing 3B(FNDC3B) |
| FNDC3B | AK075220 | Homo sapiens | fibronectin type III domain containing 3B(FNDC3B) |
| FNDC3B | NM_001135095 | Homo sapiens | fibronectin type III domain containing 3B(FNDC3B) |
| FNDC3B | XM_017007064 | Homo sapiens | fibronectin type III domain containing 3B(FNDC3B) |
| FNDC3B | BX648340 | Homo sapiens | fibronectin type III domain containing 3B(FNDC3B) |
| FNDC3B | XM_017007063 | Homo sapiens | fibronectin type III domain containing 3B(FNDC3B) |
| FNDC3B | AI417065 | Homo sapiens | fibronectin type III domain containing 3B(FNDC3B) |
| FNDC3B | AK223599 | Homo sapiens | fibronectin type III domain containing 3B(FNDC3B) |
| FNDC3B | XM_017007062 | Homo sapiens | fibronectin type III domain containing 3B(FNDC3B) |
| FNDC3B | AK127826 | Homo sapiens | fibronectin type III domain containing 3B(FNDC3B) |
| FNDC3B | BC012204 | Homo sapiens | fibronectin type III domain containing 3B(FNDC3B) |
| FNDC3B | AB098597 | Homo sapiens | fibronectin type III domain containing 3B(FNDC3B) |
| FNDC3B | AL157482 | Homo sapiens | fibronectin type III domain containing 3B(FNDC3B) |
| FNDC3B | BC026005 | Homo sapiens | fibronectin type III domain containing 3B(FNDC3B) |
| FNDC3B | AY358367 | Homo sapiens | fibronectin type III domain containing 3B(FNDC3B) |
| FNDC3B | AY358146 | Homo sapiens | fibronectin type III domain containing 3B(FNDC3B) |
| FNDC3B | AK092465 | Homo sapiens | fibronectin type III domain containing 3B(FNDC3B) |
| FNDC3B | BC033635 | Homo sapiens | fibronectin type III domain containing 3B(FNDC3B) |
| FNDC3B | AK314478 | Homo sapiens | fibronectin type III domain containing 3B(FNDC3B) |
| FNDC3B | BC039297 | Homo sapiens | fibronectin type III domain containing 3B(FNDC3B) |
| FNDC3B | BX648415 | Homo sapiens | fibronectin type III domain containing 3B(FNDC3B) |
| DYRK1A | AB015284 | Homo sapiens | dual specificity tyrosine phosphorylation regulated kinase 1A(DYRK1A) |
| DYRK1A | NM_101395 | Homo sapiens | dual specificity tyrosine phosphorylation regulated kinase 1A(DYRK1A) |
| DYRK1A | XM_017028284 | Homo sapiens | dual specificity tyrosine phosphorylation regulated kinase 1A(DYRK1A) |
| DYRK1A | AB015283 | Homo sapiens | dual specificity tyrosine phosphorylation regulated kinase 1A(DYRK1A) |
| DYRK1A | XM_017028285 | Homo sapiens | dual specificity tyrosine phosphorylation regulated kinase 1A(DYRK1A) |
| DYRK1A | AB015282 | Homo sapiens | dual specificity tyrosine phosphorylation regulated kinase 1A(DYRK1A) |
| DYRK1A | XM_017028286 | Homo sapiens | dual specificity tyrosine phosphorylation regulated kinase 1A(DYRK1A) |
| DYRK1A | BC045802 | Homo sapiens | dual specificity tyrosine phosphorylation regulated kinase 1A(DYRK1A) |
| DYRK1A | U58496 | Homo sapiens | dual specificity tyrosine phosphorylation regulated kinase 1A(DYRK1A) |
| DYRK1A | NM_130438 | Homo sapiens | dual specificity tyrosine phosphorylation regulated kinase 1A(DYRK1A) |
| DYRK1A | D85759 | Homo sapiens | dual specificity tyrosine phosphorylation regulated kinase 1A(DYRK1A) |
| DYRK1A | NM_130437 | Homo sapiens | dual specificity tyrosine phosphorylation regulated kinase 1A(DYRK1A) |
| DYRK1A | NM_130436 | Homo sapiens | dual specificity tyrosine phosphorylation regulated kinase 1A(DYRK1A) |
| DYRK1A | U52373 | Homo sapiens | dual specificity tyrosine phosphorylation regulated kinase 1A(DYRK1A) |
| DYRK1A | Z25423 | Homo sapiens | dual specificity tyrosine phosphorylation regulated kinase 1A(DYRK1A) |
| DYRK1A | BC065184 | Homo sapiens | dual specificity tyrosine phosphorylation regulated kinase 1A(DYRK1A) |
| DYRK1A | NM_001396 | Homo sapiens | dual specificity tyrosine phosphorylation regulated kinase 1A(DYRK1A) |
| DYRK1A | BC156309 | Homo sapiens | dual specificity tyrosine phosphorylation regulated kinase 1A(DYRK1A) |
| DYRK1A | XM_011529485 | Homo sapiens | dual specificity tyrosine phosphorylation regulated kinase 1A(DYRK1A) |
| DYRK1A | AF108830 | Homo sapiens | dual specificity tyrosine phosphorylation regulated kinase 1A(DYRK1A) |
| DYRK1A | XM_011529484 | Homo sapiens | dual specificity tyrosine phosphorylation regulated kinase 1A(DYRK1A) |
| DYRK1A | XM_011529483 | Homo sapiens | dual specificity tyrosine phosphorylation regulated kinase 1A(DYRK1A) |
| DYRK1A | XM_011529482 | Homo sapiens | dual specificity tyrosine phosphorylation regulated kinase 1A(DYRK1A) |
| DYRK1A | XM_006723976 | Homo sapiens | dual specificity tyrosine phosphorylation regulated kinase 1A(DYRK1A) |
| DYRK1A | XM_006723977 | Homo sapiens | dual specificity tyrosine phosphorylation regulated kinase 1A(DYRK1A) |
| DYRK1A | XM_006723978 | Homo sapiens | dual specificity tyrosine phosphorylation regulated kinase 1A(DYRK1A) |
| DYRK1A | XM_006723979 | Homo sapiens | dual specificity tyrosine phosphorylation regulated kinase 1A(DYRK1A) |
| DYRK1A | BC172505 | Homo sapiens | dual specificity tyrosine phosphorylation regulated kinase 1A(DYRK1A) |
| DYRK1A | HF584752 | Homo sapiens | dual specificity tyrosine phosphorylation regulated kinase 1A(DYRK1A) |
| DYRK1A | HF584751 | Homo sapiens | dual specificity tyrosine phosphorylation regulated kinase 1A(DYRK1A) |
| DYRK1A | AK301752 | Homo sapiens | dual specificity tyrosine phosphorylation regulated kinase 1A(DYRK1A) |
| DYRK1A | XM_005260933 | Homo sapiens | dual specificity tyrosine phosphorylation regulated kinase 1A(DYRK1A) |
| DYRK1A | BC030515 | Homo sapiens | dual specificity tyrosine phosphorylation regulated kinase 1A(DYRK1A) |
| DYRK1A | AJ001870 | Homo sapiens | dual specificity tyrosine phosphorylation regulated kinase 1A(DYRK1A) |
| DYRK1A | D86550 | Homo sapiens | dual specificity tyrosine phosphorylation regulated kinase 1A(DYRK1A) |
| DYRK1A | AJ001871 | Homo sapiens | dual specificity tyrosine phosphorylation regulated kinase 1A(DYRK1A) |
| MITF | AB006909 | Homo sapiens | melanogenesis associated transcription factor(MITF) |
| MITF | BQ219650 | Homo sapiens | melanogenesis associated transcription factor(MITF) |
| MITF | AL110195 | Homo sapiens | melanogenesis associated transcription factor(MITF) |
| MITF | BC012503 | Homo sapiens | melanogenesis associated transcription factor(MITF) |
| MITF | AB006988 | Homo sapiens | melanogenesis associated transcription factor(MITF) |
| MITF | NM_001184967 | Homo sapiens | melanogenesis associated transcription factor(MITF) |
| MITF | AB006989 | Homo sapiens | melanogenesis associated transcription factor(MITF) |
| MITF | NM_001184968 | Homo sapiens | melanogenesis associated transcription factor(MITF) |
| MITF | BC026961 | Homo sapiens | melanogenesis associated transcription factor(MITF) |
| MITF | AY632572 | Homo sapiens | melanogenesis associated transcription factor(MITF) |
| MITF | AK291318 | Homo sapiens | melanogenesis associated transcription factor(MITF) |
| MITF | AY632574 | Homo sapiens | melanogenesis associated transcription factor(MITF) |
| MITF | AK297858 | Homo sapiens | melanogenesis associated transcription factor(MITF) |
| MITF | NM_198159 | Homo sapiens | melanogenesis associated transcription factor(MITF) |
| MITF | NM_198158 | Homo sapiens | melanogenesis associated transcription factor(MITF) |
| MITF | BU167035 | Homo sapiens | melanogenesis associated transcription factor(MITF) |
| MITF | AK296129 | Homo sapiens | melanogenesis associated transcription factor(MITF) |
| MITF | NM_198178 | Homo sapiens | melanogenesis associated transcription factor(MITF) |
| MITF | NM_198177 | Homo sapiens | melanogenesis associated transcription factor(MITF) |
| MITF | GU355676 | Homo sapiens | melanogenesis associated transcription factor(MITF) |
| MITF | BC011461 | Homo sapiens | melanogenesis associated transcription factor(MITF) |
| MITF | NM_000248 | Homo sapiens | melanogenesis associated transcription factor(MITF) |
| MITF | XM_005264755 | Homo sapiens | melanogenesis associated transcription factor(MITF) |
| MITF | AB061771 | Homo sapiens | melanogenesis associated transcription factor(MITF) |
| MITF | AL117653 | Homo sapiens | melanogenesis associated transcription factor(MITF) |
| MITF | XM_005264754 | Homo sapiens | melanogenesis associated transcription factor(MITF) |
| MITF | XM_017006448 | Homo sapiens | melanogenesis associated transcription factor(MITF) |
| MITF | BC065243 | Homo sapiens | melanogenesis associated transcription factor(MITF) |
| MITF | XM_017006447 | Homo sapiens | melanogenesis associated transcription factor(MITF) |
| MITF | AW242257 | Homo sapiens | melanogenesis associated transcription factor(MITF) |
| MITF | NM_006722 | Homo sapiens | melanogenesis associated transcription factor(MITF) |
| MITF | XM_017006446 | Homo sapiens | melanogenesis associated transcription factor(MITF) |
| MITF | XM_017006445 | Homo sapiens | melanogenesis associated transcription factor(MITF) |
| MITF | XM_017006444 | Homo sapiens | melanogenesis associated transcription factor(MITF) |
| MITF | DC388606 | Homo sapiens | melanogenesis associated transcription factor(MITF) |
| MITF | DA058963 | Homo sapiens | melanogenesis associated transcription factor(MITF) |
| MITF | BM800230 | Homo sapiens | melanogenesis associated transcription factor(MITF) |
| MITF | XM_011533725 | Homo sapiens | melanogenesis associated transcription factor(MITF) |
| MITF | Z29678 | Homo sapiens | melanogenesis associated transcription factor(MITF) |
| MITF | XM_011533722 | Homo sapiens | melanogenesis associated transcription factor(MITF) |
| MITF | XM_011533723 | Homo sapiens | melanogenesis associated transcription factor(MITF) |
| MITF | XM_006713164 | Homo sapiens | melanogenesis associated transcription factor(MITF) |
| MITF | XM_011533726 | Homo sapiens | melanogenesis associated transcription factor(MITF) |
| PAX8-AS1 | AK056355 | Homo sapiens | PAX8 antisense RNA 1(PAX8-AS1) |
| PAX8-AS1 | BF056746 | Homo sapiens | PAX8 antisense RNA 1(PAX8-AS1) |
| PAX8-AS1 | AK310158 | Homo sapiens | PAX8 antisense RNA 1(PAX8-AS1) |
| PAX8-AS1 | NR_015377 | Homo sapiens | PAX8 antisense RNA 1(PAX8-AS1) |
| PAX8-AS1 | AK307781 | Homo sapiens | PAX8 antisense RNA 1(PAX8-AS1) |
| PAX8-AS1 | AY007128 | Homo sapiens | PAX8 antisense RNA 1(PAX8-AS1) |
| PAX8-AS1 | BC042373 | Homo sapiens | PAX8 antisense RNA 1(PAX8-AS1) |
| PAX8-AS1 | AK056052 | Homo sapiens | PAX8 antisense RNA 1(PAX8-AS1) |
| PAX8-AS1 | LK937783 | Homo sapiens | PAX8 antisense RNA 1(PAX8-AS1) |
| PAX8-AS1 | AK130275 | Homo sapiens | PAX8 antisense RNA 1(PAX8-AS1) |
| PAX8-AS1 | AK126431 | Homo sapiens | PAX8 antisense RNA 1(PAX8-AS1) |
| PAX8-AS1 | AK001856 | Homo sapiens | PAX8 antisense RNA 1(PAX8-AS1) |
| PAX8-AS1 | AL390179 | Homo sapiens | PAX8 antisense RNA 1(PAX8-AS1) |
| PAX8-AS1 | BX537688 | Homo sapiens | PAX8 antisense RNA 1(PAX8-AS1) |
| PAX8-AS1 | CA416382 | Homo sapiens | PAX8 antisense RNA 1(PAX8-AS1) |
| PAX8-AS1 | BC036699 | Homo sapiens | PAX8 antisense RNA 1(PAX8-AS1) |
| PAX8-AS1 | BC033562 | Homo sapiens | PAX8 antisense RNA 1(PAX8-AS1) |
| PAX8-AS1 | DA573927 | Homo sapiens | PAX8 antisense RNA 1(PAX8-AS1) |
| PAX8-AS1 | NR_047570 | Homo sapiens | PAX8 antisense RNA 1(PAX8-AS1) |
| NEAT1 | LK938844 | Homo sapiens | nuclear paraspeckle assembly transcript 1 (non-protein coding)(NEAT1) |
| NEAT1 | LK938846 | Homo sapiens | nuclear paraspeckle assembly transcript 1 (non-protein coding)(NEAT1) |
| NEAT1 | HG503866 | Homo sapiens | nuclear paraspeckle assembly transcript 1 (non-protein coding)(NEAT1) |
| NEAT1 | LK938845 | Homo sapiens | nuclear paraspeckle assembly transcript 1 (non-protein coding)(NEAT1) |
| NEAT1 | EF177379 | Homo sapiens | nuclear paraspeckle assembly transcript 1 (non-protein coding)(NEAT1) |
| NEAT1 | AI590745 | Homo sapiens | nuclear paraspeckle assembly transcript 1 (non-protein coding)(NEAT1) |
| NEAT1 | NR_002802 | Homo sapiens | nuclear paraspeckle assembly transcript 1 (non-protein coding)(NEAT1) |
| NEAT1 | AF508303 | Homo sapiens | nuclear paraspeckle assembly transcript 1 (non-protein coding)(NEAT1) |
| NEAT1 | NR_131012 | Homo sapiens | nuclear paraspeckle assembly transcript 1 (non-protein coding)(NEAT1) |
| NEAT1 | AF080092 | Homo sapiens | nuclear paraspeckle assembly transcript 1 (non-protein coding)(NEAT1) |
| NEAT1 | GQ859162 | Homo sapiens | nuclear paraspeckle assembly transcript 1 (non-protein coding)(NEAT1) |
| NEAT1 | AK027191 | Homo sapiens | nuclear paraspeckle assembly transcript 1 (non-protein coding)(NEAT1) |
| NEAT1 | HG503867 | Homo sapiens | nuclear paraspeckle assembly transcript 1 (non-protein coding)(NEAT1) |
| NEAT1 | U60873 | Homo sapiens | nuclear paraspeckle assembly transcript 1 (non-protein coding)(NEAT1) |
| NEAT1 | NR_028272 | Homo sapiens | nuclear paraspeckle assembly transcript 1 (non-protein coding)(NEAT1) |
| SOX10 | CR536571 | Homo sapiens | SRY-box 10(SOX10) |
| SOX10 | BT020029 | Homo sapiens | SRY-box 10(SOX10) |
| SOX10 | CR456584 | Homo sapiens | SRY-box 10(SOX10) |
| SOX10 | NM_006941 | Homo sapiens | SRY-box 10(SOX10) |
| SOX10 | BC007595 | Homo sapiens | SRY-box 10(SOX10) |
| SOX10 | BC018808 | Homo sapiens | SRY-box 10(SOX10) |
| SOX10 | AK300945 | Homo sapiens | SRY-box 10(SOX10) |
| SOX10 | DQ896471 | Homo sapiens | SRY-box 10(SOX10) |
| SOX10 | DQ893172 | Homo sapiens | SRY-box 10(SOX10) |
| SOX10 | AJ001183 | Homo sapiens | SRY-box 10(SOX10) |
| SOX10 | BC002824 | Homo sapiens | SRY-box 10(SOX10) |
| SOX10 | AK310896 | Homo sapiens | SRY-box 10(SOX10) |
| SOX10 | CU013471 | Homo sapiens | SRY-box 10(SOX10) |
| SOX10 | CU013183 | Homo sapiens | SRY-box 10(SOX10) |
| RUNX2 | XR_926323 | Homo sapiens | runt related transcription factor 2(RUNX2) |
| RUNX2 | NM_001015051 | Homo sapiens | runt related transcription factor 2(RUNX2) |
| RUNX2 | NM_001024630 | Homo sapiens | runt related transcription factor 2(RUNX2) |
| RUNX2 | BC160022 | Homo sapiens | runt related transcription factor 2(RUNX2) |
| RUNX2 | BX108677 | Homo sapiens | runt related transcription factor 2(RUNX2) |
| RUNX2 | XM_006715232 | Homo sapiens | runt related transcription factor 2(RUNX2) |
| RUNX2 | AF053952 | Homo sapiens | runt related transcription factor 2(RUNX2) |
| RUNX2 | BC108919 | Homo sapiens | runt related transcription factor 2(RUNX2) |
| RUNX2 | CN431726 | Homo sapiens | runt related transcription factor 2(RUNX2) |
| RUNX2 | AL353944 | Homo sapiens | runt related transcription factor 2(RUNX2) |
| RUNX2 | L40992 | Homo sapiens | runt related transcription factor 2(RUNX2) |
| RUNX2 | NM_004348 | Homo sapiens | runt related transcription factor 2(RUNX2) |
| RUNX2 | AW469546 | Homo sapiens | runt related transcription factor 2(RUNX2) |
| RUNX2 | NM_001278478 | Homo sapiens | runt related transcription factor 2(RUNX2) |
| RUNX2 | XR_001743701 | Homo sapiens | runt related transcription factor 2(RUNX2) |
| RUNX2 | XM_011514966 | Homo sapiens | runt related transcription factor 2(RUNX2) |
| RUNX2 | AF087960 | Homo sapiens | runt related transcription factor 2(RUNX2) |
| RUNX2 | XM_011514965 | Homo sapiens | runt related transcription factor 2(RUNX2) |
| RUNX2 | XM_011514964 | Homo sapiens | runt related transcription factor 2(RUNX2) |
| RUNX2 | XM_011514963 | Homo sapiens | runt related transcription factor 2(RUNX2) |
| RUNX2 | BC108920 | Homo sapiens | runt related transcription factor 2(RUNX2) |
| RUNX2 | XM_011514962 | Homo sapiens | runt related transcription factor 2(RUNX2) |
| RUNX2 | XM_011514961 | Homo sapiens | runt related transcription factor 2(RUNX2) |
| RUNX2 | XM_017011391 | Homo sapiens | runt related transcription factor 2(RUNX2) |
| RUNX2 | XM_011514960 | Homo sapiens | runt related transcription factor 2(RUNX2) |
| RUNX2 | NR_103533 | Homo sapiens | runt related transcription factor 2(RUNX2) |
| RUNX2 | XM_017011394 | Homo sapiens | runt related transcription factor 2(RUNX2) |
| RUNX2 | XM_017011395 | Homo sapiens | runt related transcription factor 2(RUNX2) |
| RUNX2 | XM_017011392 | Homo sapiens | runt related transcription factor 2(RUNX2) |
| RUNX2 | XM_017011393 | Homo sapiens | runt related transcription factor 2(RUNX2) |
| RUNX2 | CD001961 | Homo sapiens | runt related transcription factor 2(RUNX2) |
| RUNX2 | DR005078 | Homo sapiens | runt related transcription factor 2(RUNX2) |
| RUNX2 | XM_017011396 | Homo sapiens | runt related transcription factor 2(RUNX2) |
| RUNX2 | NR_103532 | Homo sapiens | runt related transcription factor 2(RUNX2) |
| MYL4 | XM_005257391 | Homo sapiens | myosin light chain 4(MYL4) |
| MYL4 | XM_011524839 | Homo sapiens | myosin light chain 4(MYL4) |
| MYL4 | NM_002476 | Homo sapiens | myosin light chain 4(MYL4) |
| MYL4 | XM_017024683 | Homo sapiens | myosin light chain 4(MYL4) |
| MYL4 | X13955 | Homo sapiens | myosin light chain 4(MYL4) |
| MYL4 | X52005 | Homo sapiens | myosin light chain 4(MYL4) |
| MYL4 | AF116676 | Homo sapiens | myosin light chain 4(MYL4) |
| MYL4 | XM_017024684 | Homo sapiens | myosin light chain 4(MYL4) |
| MYL4 | BC030228 | Homo sapiens | myosin light chain 4(MYL4) |
| MYL4 | M36172 | Homo sapiens | myosin light chain 4(MYL4) |
| MYL4 | H83803 | Homo sapiens | myosin light chain 4(MYL4) |
| MYL4 | M20641 | Homo sapiens | myosin light chain 4(MYL4) |
| MYL4 | AM392902 | Homo sapiens | myosin light chain 4(MYL4) |
| MYL4 | AM392536 | Homo sapiens | myosin light chain 4(MYL4) |
| MYL4 | AM392523 | Homo sapiens | myosin light chain 4(MYL4) |
| MYL4 | AM393643 | Homo sapiens | myosin light chain 4(MYL4) |
| MYL4 | NM_001002841 | Homo sapiens | myosin light chain 4(MYL4) |
| MYL4 | AM393677 | Homo sapiens | myosin light chain 4(MYL4) |
| MYL4 | BU658678 | Homo sapiens | myosin light chain 4(MYL4) |
| MYL4 | M24121 | Homo sapiens | myosin light chain 4(MYL4) |
| LMX1A | NM_177399 | Homo sapiens | LIM homeobox transcription factor 1 alpha(LMX1A) |
| LMX1A | NM_177398 | Homo sapiens | LIM homeobox transcription factor 1 alpha(LMX1A) |
| LMX1A | AK122800 | Homo sapiens | LIM homeobox transcription factor 1 alpha(LMX1A) |
| LMX1A | NM_001033507 | Homo sapiens | LIM homeobox transcription factor 1 alpha(LMX1A) |
| LMX1A | BC119744 | Homo sapiens | LIM homeobox transcription factor 1 alpha(LMX1A) |
| LMX1A | BC119743 | Homo sapiens | LIM homeobox transcription factor 1 alpha(LMX1A) |
| LMX1A | XM_011509538 | Homo sapiens | LIM homeobox transcription factor 1 alpha(LMX1A) |
| LMX1A | BC066353 | Homo sapiens | LIM homeobox transcription factor 1 alpha(LMX1A) |
| LMX1A | NM_001174069 | Homo sapiens | LIM homeobox transcription factor 1 alpha(LMX1A) |
| LMX1A | XM_011509540 | Homo sapiens | LIM homeobox transcription factor 1 alpha(LMX1A) |
| LMX1A | BM678780 | Homo sapiens | LIM homeobox transcription factor 1 alpha(LMX1A) |
| LMX1A | AK127724 | Homo sapiens | LIM homeobox transcription factor 1 alpha(LMX1A) |
| LMX1A | JF432394 | Homo sapiens | LIM homeobox transcription factor 1 alpha(LMX1A) |
| LMX1A | BC160062 | Homo sapiens | LIM homeobox transcription factor 1 alpha(LMX1A) |
| PSME4 | XM_011532709 | Homo sapiens | proteasome activator subunit 4(PSME4) |
| PSME4 | AK026085 | Homo sapiens | proteasome activator subunit 4(PSME4) |
| PSME4 | XM_011532707 | Homo sapiens | proteasome activator subunit4(PSME4) |
| PSME4 | AA884260 | Homo sapiens | proteasome activator subunit 4(PSME4) |
| PSME4 | BC043602 | Homo sapiens | proteasome activator subunit 4(PSME4) |
| PSME4 | AK124923 | Homo sapiens | proteasome activator subunit 4(PSME4) |
| PSME4 | AU117034 | Homo sapiens | proteasome activator subunit 4(PSME4) |
| PSME4 | AB621805 | Homo sapiens | proteasome activator subunit 4(PSME4) |
| PSME4 | AL045471 | Homo sapiens | proteasome activator subunit 4(PSME4) |
| PSME4 | BC143737 | Homo sapiens | proteasome activator subunit 4(PSME4) |
| PSME4 | BC062760 | Homo sapiens | proteasome activator subunit 4(PSME4) |
| PSME4 | BC143739 | Homo sapiens | proteasome activator subunit 4(PSME4) |
| PSME4 | NM_014614 | Homo sapiens | proteasome activator subunit 4(PSME4) |
| PSME4 | AK025517 | Homo sapiens | proteasome activator subunit 4(PSME4) |
| PSME4 | BC112169 | Homo sapiens | proteasome activator subunit 4(PSME4) |
| PSME4 | AL599601 | Homo sapiens | proteasome activator subunit 4(PSME4) |
| PSME4 | AY894754 | Homo sapiens | proteasome activator subunit 4(PSME4) |
| PSME4 | D38521 | Homo sapiens | proteasome activator subunit 4(PSME4) |
| PSME4 | XM_011532705 | Homo sapiens | proteasome activator subunit 4(PSME4) |
| PSME4 | XM_011532706 | Homo sapiens | proteasome activator subunit 4(PSME4) |
| PSME4 | XR_001738679 | Homo sapiens | proteasome activator subunit 4(PSME4) |
| PSME4 | AY894755 | Homo sapiens | proteasome activator subunit 4(PSME4) |
| PSME4 | AY894756 | Homo sapiens | proteasome activator subunit 4(PSME4) |
| PSME4 | BC071768 | Homo sapiens | proteasome activator subunit 4(PSME4) |
| PSME4 | BQ898809 | Homo sapiens | proteasome activator subunit 4(PSME4) |
| PSME4 | XM_006711969 | Homo sapiens | proteasome activator subunit 4(PSME4) |
| PSME4 | BU569553 | Homo sapiens | proteasome activator subunit 4(PSME4) |
| PSME4 | BC032418 | Homo sapiens | proteasome activator subunit 4(PSME4) |
| PSME4 | BC017090 | Homo sapiens | proteasome activator subunit 4(PSME4) |
| PSME4 | BC113668 | Homo sapiens | proteasome activator subunit 4(PSME4) |
| CFTR | BC156254 | Homo sapiens | cystic fibrosis transmembrane conductance regulator(CFTR) |
| CFTR | XM_017011699 | Homo sapiens | cystic fibrosis transmembrane conductance regulator(CFTR) |
| CFTR | X73053 | Homo sapiens | cystic fibrosis transmembrane conductance regulator(CFTR) |
| CFTR | XM_011515754 | Homo sapiens | cystic fibrosis transmembrane conductance regulator(CFTR) |
| CFTR | M28668 | Homo sapiens | cystic fibrosis transmembrane conductance regulator(CFTR) |
| CFTR | XM_011515753 | Homo sapiens | cystic fibrosis transmembrane conductance regulator(CFTR) |
| CFTR | S64699 | Homo sapiens | cystic fibrosis transmembrane conductance regulator(CFTR) |
| CFTR | XM_011515751 | Homo sapiens | cystic fibrosis transmembrane conductance regulator(CFTR) |
| CFTR | NM_000492 | Homo sapiens | cystic fibrosis transmembrane conductance regulator(CFTR) |
| CFTR | S82430 | Homo sapiens | cystic fibrosis transmembrane conductance regulator(CFTR) |
| CFTR | BC143713 | Homo sapiens | cystic fibrosis transmembrane conductance regulator(CFTR) |
| CNTN4-AS2 | BE041831 | Homo sapiens | CNTN4 antisense RNA 2(CNTN4-AS2) |
| CNTN4-AS2 | NR_046555 | Homo sapiens | CNTN4 antisense RNA 2(CNTN4-AS2) |
| CNTN4-AS2 | HG495393 | Homo sapiens | CNTN4 antisense RNA 2(CNTN4-AS2) |
| CHRNA1 | Y00762 | Homo sapiens | cholinergic receptor nicotinic alpha 1 subunit(CHRNA1) |
| CHRNA1 | CD013889 | Homo sapiens | cholinergic receptor nicotinic alpha 1 subunit(CHRNA1) |
| CHRNA1 | AK299445 | Homo sapiens | cholinergic receptor nicotinic alpha 1 subunit(CHRNA1) |
| CHRNA1 | NM_000079 | Homo sapiens | cholinergic receptor nicotinic alpha 1 subunit(CHRNA1) |
| CHRNA1 | XM_017003257 | Homo sapiens | cholinergic receptor nicotinic alpha 1 subunit(CHRNA1) |
| CHRNA1 | XM_017003256 | Homo sapiens | cholinergic receptor nicotinic alpha 1 subunit(CHRNA1) |
| CHRNA1 | BG828551 | Homo sapiens | cholinergic receptor nicotinic alpha 1 subunit(CHRNA1) |
| CHRNA1 | NM_001039523 | Homo sapiens | cholinergic receptor nicotinic alpha 1 subunit(CHRNA1) |
| CHRNA1 | BC043196 | Homo sapiens | cholinergic receptor nicotinic alpha 1 subunit(CHRNA1) |
| CHRNA1 | AK291338 | Homo sapiens | cholinergic receptor nicotinic alpha 1 subunit(CHRNA1) |
| CHRNA1 | BC006314 | Homo sapiens | cholinergic receptor nicotinic alpha 1 subunit(CHRNA1) |
| CHRNA1 | AK315312 | Homo sapiens | cholinergic receptor nicotinic alpha 1 subunit(CHRNA1) |
| CHRNA1 | DQ323657 | Homo sapiens | cholinergic receptor nicotinic alpha 1 subunit(CHRNA1) |
| CHRNA1 | DQ323658 | Homo sapiens | cholinergic receptor nicotinic alpha 1 subunit(CHRNA1) |
| CHRNA1 | S77094 | Homo sapiens | cholinergic receptor nicotinic alpha 1 subunit(CHRNA1) |
| NOTCH1 | AB209873 | Homo sapiens | notch 1(NOTCH1) |
| NOTCH1 | AF308602 | Homo sapiens | notch 1(NOTCH1) |
| NOTCH1 | CR457221 | Homo sapiens | notch 1(NOTCH1) |
| NOTCH1 | R42303 | Homo sapiens | notch 1(NOTCH1) |
| NOTCH1 | AK000012 | Homo sapiens | notch 1(NOTCH1) |
| NOTCH1 | BC046127 | Homo sapiens | notch 1(NOTCH1) |
| NOTCH1 | BC013208 | Homo sapiens | notch 1(NOTCH1) |
| NOTCH1 | BC049843 | Homo sapiens | notch 1(NOTCH1) |
| NOTCH1 | M73980 | Homo sapiens | notch 1(NOTCH1) |
| NOTCH1 | BC063597 | Homo sapiens | notch 1(NOTCH1) |
| NOTCH1 | CN431067 | Homo sapiens | notch 1(NOTCH1) |
| NOTCH1 | BC039147 | Homo sapiens | notch 1(NOTCH1) |
| NOTCH1 | DA324222 | Homo sapiens | notch 1(NOTCH1) |
| NOTCH1 | NM_017617 | Homo sapiens | notch 1(NOTCH1) |
| NOTCH1 | XM_011518717 | Homo sapiens | notch 1(NOTCH1) |
| PDE1A | XM_017004295 | Homo sapiens | phosphodiesterase 1A(PDE1A) |
| PDE1A | XM_017004296 | Homo sapiens | phosphodiesterase 1A(PDE1A) |
| PDE1A | DA128734 | Homo sapiens | phosphodiesterase 1A(PDE1A) |
| PDE1A | XM_017004294 | Homo sapiens | phosphodiesterase 1A(PDE1A) |
| PDE1A | AF110240 | Homo sapiens | phosphodiesterase 1A(PDE1A) |
| PDE1A | BG196993 | Homo sapiens | phosphodiesterase 1A(PDE1A) |
| PDE1A | AK301720 | Homo sapiens | phosphodiesterase 1A(PDE1A) |
| PDE1A | DB128091 | Homo sapiens | phosphodiesterase 1A(PDE1A) |
| PDE1A | XM_017004299 | Homo sapiens | phosphodiesterase 1A(PDE1A) |
| PDE1A | XM_017004297 | Homo sapiens | phosphodiesterase 1A(PDE1A) |
| PDE1A | XM_017004298 | Homo sapiens | phosphodiesterase 1A(PDE1A) |
| PDE1A | XM_011511323 | Homo sapiens | phosphodiesterase 1A(PDE1A) |
| PDE1A | BM719913 | Homo sapiens | phosphodiesterase 1A(PDE1A) |
| PDE1A | XM_011511325 | Homo sapiens | phosphodiesterase 1A(PDE1A) |
| PDE1A | XM_011511324 | Homo sapiens | phosphodiesterase 1A(PDE1A) |
| PDE1A | AJ401610 | Homo sapiens | phosphodiesterase 1A(PDE1A) |
| PDE1A | AK295657 | Homo sapiens | phosphodiesterase 1A(PDE1A) |
| PDE1A | DQ896694 | Homo sapiens | phosphodiesterase 1A(PDE1A) |
| PDE1A | XM_011511326 | Homo sapiens | phosphodiesterase 1A(PDE1A) |
| PDE1A | AA846454 | Homo sapiens | phosphodiesterase 1A(PDE1A) |
| PDE1A | DQ892369 | Homo sapiens | phosphodiesterase 1A(PDE1A) |
| PDE1A | NM_001003683 | Homo sapiens | phosphodiesterase 1A(PDE1A) |
| PDE1A | U40370 | Homo sapiens | phosphodiesterase 1A(PDE1A) |
| PDE1A | AL110263 | Homo sapiens | phosphodiesterase 1A(PDE1A) |
| PDE1A | NM_005019 | Homo sapiens | phosphodiesterase 1A(PDE1A) |
| PDE1A | XM_017004301 | Homo sapiens | phosphodiesterase 1A(PDE1A) |
| PDE1A | AF110238 | Homo sapiens | phosphodiesterase 1A(PDE1A) |
| PDE1A | XM_017004302 | Homo sapiens | phosphodiesterase 1A(PDE1A) |
| PDE1A | AF110237 | Homo sapiens | phosphodiesterase 1A(PDE1A) |
| PDE1A | AF110236 | Homo sapiens | phosphodiesterase 1A(PDE1A) |
| PDE1A | XM_017004300 | Homo sapiens | phosphodiesterase 1A(PDE1A) |
| PDE1A | AF110235 | Homo sapiens | phosphodiesterase 1A(PDE1A) |
| PDE1A | AL536937 | Homo sapiens | phosphodiesterase 1A(PDE1A) |
| PDE1A | BC047057 | Homo sapiens | phosphodiesterase 1A(PDE1A) |
| PDE1A | NM_001258313 | Homo sapiens | phosphodiesterase 1A(PDE1A) |
| PDE1A | DB512285 | Homo sapiens | phosphodiesterase 1A(PDE1A) |
| PDE1A | NM_001258312 | Homo sapiens | phosphodiesterase 1A(PDE1A) |
| PDE1A | BC022480 | Homo sapiens | phosphodiesterase 1A(PDE1A) |
| PDE1A | DA295167 | Homo sapiens | phosphodiesterase 1A(PDE1A) |
| PDE1A | DC396228 | Homo sapiens | phosphodiesterase 1A(PDE1A) |
| PDE1A | AK130643 | Homo sapiens | phosphodiesterase 1A(PDE1A) |
| PDE1A | NM_001258314 | Homo sapiens | phosphodiesterase 1A(PDE1A) |
| PDE1A | AK294239 | Homo sapiens | phosphodiesterase 1A(PDE1A) |
| PDE1A | AB038227 | Homo sapiens | phosphodiesterase 1A(PDE1A) |
| PDE1A | DQ893372 | Homo sapiens | phosphodiesterase 1A(PDE1A) |
| PDE1A | DQ895578 | Homo sapiens | phosphodiesterase 1A(PDE1A) |
| PDE1A | AB038228 | Homo sapiens | phosphodiesterase 1A(PDE1A) |
| PDE1A | XR_001738769 | Homo sapiens | phosphodiesterase 1A(PDE1A) |
| ARX | BC169333 | Homo sapiens | aristaless related homeobox(ARX) |
| ARX | BC169334 | Homo sapiens | aristaless related homeobox(ARX) |
| ARX | AY038071 | Homo sapiens | aristaless related homeobox(ARX) |
| ARX | AA484051 | Homo sapiens | aristaless related homeobox(ARX) |
| ARX | BQ269551 | Homo sapiens | aristaless related homeobox(ARX) |
| ARX | NM_139058 | Homo sapiens | aristaless related homeobox(ARX) |
| ARX | BQ100952 | Homo sapiens | aristaless related homeobox(ARX) |
| ARX | BF196892 | Homo sapiens | aristaless related homeobox(ARX) |
| ARX | CA775911 | Homo sapiens | aristaless related homeobox(ARX) |
| GLIS3 | DQ438889 | Homo sapiens | GLIS family zinc finger 3(GLIS3) |
| GLIS3 | DQ438888 | Homo sapiens | GLIS family zinc finger 3(GLIS3) |
| GLIS3 | DQ438885 | Homo sapiens | GLIS family zinc finger 3(GLIS3) |
| GLIS3 | DQ438884 | Homo sapiens | GLIS family zinc finger 3(GLIS3) |
| GLIS3 | DQ438887 | Homo sapiens | GLIS family zinc finger 3(GLIS3) |
| GLIS3 | DQ438886 | Homo sapiens | GLIS family zinc finger 3(GLIS3) |
| GLIS3 | DQ438881 | Homo sapiens | GLIS family zinc finger 3(GLIS3) |
| GLIS3 | DQ438880 | Homo sapiens | GLIS family zinc finger 3(GLIS3) |
| GLIS3 | DQ438883 | Homo sapiens | GLIS family zinc finger 3(GLIS3) |
| GLIS3 | DQ438882 | Homo sapiens | GLIS family zinc finger 3(GLIS3) |
| GLIS3 | NM_152629 | Homo sapiens | GLIS family zinc finger 3(GLIS3) |
| GLIS3 | AK096318 | Homo sapiens | GLIS family zinc finger 3(GLIS3) |
| GLIS3 | AK075059 | Homo sapiens | GLIS family zinc finger 3(GLIS3) |
| GLIS3 | XM_011517764 | Homo sapiens | GLIS family zinc finger 3(GLIS3) |
| GLIS3 | XM_011517765 | Homo sapiens | GLIS family zinc finger 3(GLIS3) |
| GLIS3 | XM_011517766 | Homo sapiens | GLIS family zinc finger 3(GLIS3) |
| GLIS3 | XM_011517767 | Homo sapiens | GLIS family zinc finger 3(GLIS3) |
| GLIS3 | XM_011517769 | Homo sapiens | GLIS family zinc finger 3(GLIS3) |
| GLIS3 | DQ438899 | Homo sapiens | GLIS family zinc finger 3(GLIS3) |
| GLIS3 | DQ438896 | Homo sapiens | GLIS family zinc finger 3(GLIS3) |
| GLIS3 | DQ438895 | Homo sapiens | GLIS family zinc finger 3(GLIS3) |
| GLIS3 | KU178885 | Homo sapiens | GLIS family zinc finger 3(GLIS3) |
| GLIS3 | DQ438898 | Homo sapiens | GLIS family zinc finger 3(GLIS3) |
| GLIS3 | KU178886 | Homo sapiens | GLIS family zinc finger 3(GLIS3) |
| GLIS3 | DQ438897 | Homo sapiens | GLIS family zinc finger 3(GLIS3) |
| GLIS3 | KU178887 | Homo sapiens | GLIS family zinc finger 3(GLIS3) |
| GLIS3 | DQ438892 | Homo sapiens | GLIS family zinc finger 3(GLIS3) |
| GLIS3 | XM_005251387 | Homo sapiens | GLIS family zinc finger 3(GLIS3) |
| GLIS3 | DQ438891 | Homo sapiens | GLIS family zinc finger 3(GLIS3) |
| GLIS3 | XM_005251386 | Homo sapiens | GLIS family zinc finger 3(GLIS3) |
| GLIS3 | DQ438894 | Homo sapiens | GLIS family zinc finger 3(GLIS3) |
| GLIS3 | DQ438893 | Homo sapiens | GLIS family zinc finger 3(GLIS3) |
| GLIS3 | DQ438890 | Homo sapiens | GLIS family zinc finger 3(GLIS3) |
| GLIS3 | XM_017014361 | Homo sapiens | GLIS family zinc finger 3(GLIS3) |
| GLIS3 | XM_005251389 | Homo sapiens | GLIS family zinc finger 3(GLIS3) |
| GLIS3 | XM_005251388 | Homo sapiens | GLIS family zinc finger 3(GLIS3) |
| GLIS3 | EU446681 | Homo sapiens | GLIS family zinc finger 3(GLIS3) |
| GLIS3 | DQ438900 | Homo sapiens | GLIS family zinc finger 3(GLIS3) |
| GLIS3 | AB065086 | Homo sapiens | GLIS family zinc finger 3(GLIS3) |
| GLIS3 | DQ438902 | Homo sapiens | GLIS family zinc finger 3(GLIS3) |
| GLIS3 | DQ438901 | Homo sapiens | GLIS family zinc finger 3(GLIS3) |
| GLIS3 | AA933816 | Homo sapiens | GLIS family zinc finger 3(GLIS3) |
| GLIS3 | AK055907 | Homo sapiens | GLIS family zinc finger 3(GLIS3) |
| GLIS3 | BC033899 | Homo sapiens | GLIS family zinc finger 3(GLIS3) |
| GLIS3 | DQ438907 | Homo sapiens | GLIS family zinc finger 3(GLIS3) |
| GLIS3 | HG501866 | Homo sapiens | GLIS family zinc finger 3(GLIS3) |
| GLIS3 | DQ438904 | Homo sapiens | GLIS family zinc finger 3(GLIS3) |
| GLIS3 | DQ438903 | Homo sapiens | GLIS family zinc finger 3(GLIS3) |
| GLIS3 | DQ438906 | Homo sapiens | GLIS family zinc finger 3(GLIS3) |
| GLIS3 | DQ438905 | Homo sapiens | GLIS family zinc finger 3(GLIS3) |
| GLIS3 | DQ438878 | Homo sapiens | GLIS family zinc finger 3(GLIS3) |
| GLIS3 | DQ438877 | Homo sapiens | GLIS family zinc finger 3(GLIS3) |
| GLIS3 | DQ438879 | Homo sapiens | GLIS family zinc finger 3(GLIS3) |
| GLIS3 | AB209404 | Homo sapiens | GLIS family zinc finger 3(GLIS3) |
| GLIS3 | XM_011517763 | Homo sapiens | GLIS family zinc finger 3(GLIS3) |
| GLIS3 | XR_929206 | Homo sapiens | GLIS family zinc finger 3(GLIS3) |
| GLIS3 | XM_006716731 | Homo sapiens | GLIS family zinc finger 3(GLIS3) |
| GLIS3 | NM_001042413 | Homo sapiens | GLIS family zinc finger 3(GLIS3) |
| TWIST1 | Y11177 | Homo sapiens | twist family bHLH transcription factor 1(TWIST1) |
| TWIST1 | Y11178 | Homo sapiens | twist family bHLH transcription factor 1(TWIST1) |
| TWIST1 | AW173505 | Homo sapiens | twist family bHLH transcription factor 1(TWIST1) |
| TWIST1 | X99268 | Homo sapiens | twist family bHLH transcription factor 1(TWIST1) |
| TWIST1 | BC036704 | Homo sapiens | twist family bHLH transcription factor 1(TWIST1) |
| TWIST1 | NM_000474 | Homo sapiens | twist family bHLH transcription factor 1(TWIST1) |
| TWIST1 | XM_011515496 | Homo sapiens | twist family bHLH transcription factor 1(TWIST1) |
| TWIST1 | DQ896770 | Homo sapiens | twist family bHLH transcription factor 1(TWIST1) |
| PHYHIPL | DB551000 | Homo sapiens | phytanoyl-CoA 2-hydroxylase interacting protein like(PHYHIPL) |
| PHYHIPL | BC011268 | Homo sapiens | phytanoyl-CoA 2-hydroxylase interacting protein like(PHYHIPL) |
| PHYHIPL | AM393181 | Homo sapiens | phytanoyl-CoA 2-hydroxylase interacting protein like(PHYHIPL) |
| PHYHIPL | DB455338 | Homo sapiens | phytanoyl-CoA 2-hydroxylase interacting protein like(PHYHIPL) |
| PHYHIPL | AY358162 | Homo sapiens | phytanoyl-CoA 2-hydroxylase interacting protein like(PHYHIPL) |
| PHYHIPL | AW299583 | Homo sapiens | phytanoyl-CoA 2-hydroxylase interacting protein like(PHYHIPL) |
| PHYHIPL | NM_001143774 | Homo sapiens | phytanoyl-CoA 2-hydroxylase interacting protein like(PHYHIPL) |
| PHYHIPL | AB058699 | Homo sapiens | phytanoyl-CoA 2-hydroxylase interacting protein like(PHYHIPL) |
| PHYHIPL | CR749429 | Homo sapiens | phytanoyl-CoA 2-hydroxylase interacting protein like(PHYHIPL) |
| PHYHIPL | AL365474 | Homo sapiens | phytanoyl-CoA 2-hydroxylase interacting protein like(PHYHIPL) |
| PHYHIPL | AL834339 | Homo sapiens | phytanoyl-CoA 2-hydroxylase interacting protein like(PHYHIPL) |
| PHYHIPL | AM392743 | Homo sapiens | phytanoyl-CoA 2-hydroxylase interacting protein like(PHYHIPL) |
| PHYHIPL | BM676577 | Homo sapiens | phytanoyl-CoA 2-hydroxylase interacting protein like(PHYHIPL) |
| PHYHIPL | NM_032439 | Homo sapiens | phytanoyl-CoA 2-hydroxylase interacting protein like(PHYHIPL) |
| PHYHIPL | XM_011540276 | Homo sapiens | phytanoyl-CoA 2-hydroxylase interacting protein like(PHYHIPL) |
| PHYHIPL | XM_011540275 | Homo sapiens | phytanoyl-CoA 2-hydroxylase interacting protein like(PHYHIPL) |
| PHYHIPL | XM_017016783 | Homo sapiens | phytanoyl-CoA 2-hydroxylase interacting protein like(PHYHIPL) |
| PHYHIPL | XM_017016782 | Homo sapiens | phytanoyl-CoA 2-hydroxylase interacting protein like(PHYHIPL) |
| PHYHIPL | AK054956 | Homo sapiens | phytanoyl-CoA 2-hydroxylase interacting protein like(PHYHIPL) |
| TRPM1 | BU933033 | Homo sapiens | transient receptor potential cation channel subfamily M member 1(TRPM1) |
| TRPM1 | BC058286 | Homo sapiens | transient receptor potential cation channel subfamily M member 1(TRPM1) |
| TRPM1 | GQ502181 | Homo sapiens | transient receptor potential cation channel subfamily M member 1(TRPM1) |
| TRPM1 | BC005892 | Homo sapiens | transient receptor potential cation channel subfamily M member 1(TRPM1) |
| TRPM1 | AB115498 | Homo sapiens | transient receptor potential cation channel subfamily M member 1(TRPM1) |
| TRPM1 | AB115500 | Homo sapiens | transient receptor potential cation channel subfamily M member 1(TRPM1) |
| TRPM1 | NM_001252030 | Homo sapiens | transient receptor potential cation channel subfamily M member 1(TRPM1) |
| TRPM1 | AB115501 | Homo sapiens | transient receptor potential cation channel subfamily M member 1(TRPM1) |
| TRPM1 | BC156069 | Homo sapiens | transient receptor potential cation channel subfamily M member 1(TRPM1) |
| TRPM1 | AB115502 | Homo sapiens | transient receptor potential cation channel subfamily M member 1(TRPM1) |
| TRPM1 | GU576175 | Homo sapiens | transient receptor potential cation channel subfamily M member 1(TRPM1) |
| TRPM1 | N42519 | Homo sapiens | transient receptor potential cation channel subfamily M member 1(TRPM1) |
| TRPM1 | BM695497 | Homo sapiens | transient receptor potential cation channel subfamily M member 1(TRPM1) |
| TRPM1 | NM_002420 | Homo sapiens | transient receptor potential cation channel subfamily M member 1(TRPM1) |
| TRPM1 | NM_001252024 | Homo sapiens | transient receptor potential cation channel subfamily M member 1(TRPM1) |
| TRPM1 | BC017849 | Homo sapiens | transient receptor potential cation channel subfamily M member 1(TRPM1) |
| TRPM1 | NM_001252020 | Homo sapiens | transient receptor potential cation channel subfamily M member 1(TRPM1) |
| TRPM1 | AB115499 | Homo sapiens | transient receptor potential cation channel subfamily M member 1(TRPM1) |
| TRPM1 | BC033627 | Homo sapiens | transient receptor potential cation channel subfamily M member 1(TRPM1) |
| TRPM1 | AF071787 | Homo sapiens | transient receptor potential cation channel subfamily M member 1(TRPM1) |
| TRPM1 | HM135791 | Homo sapiens | transient receptor potential cation channel subfamily M member 1(TRPM1) |
| TRPM1 | BC070356 | Homo sapiens | transient receptor potential cation channel subfamily M member 1(TRPM1) |
| TRPM1 | HM135790 | Homo sapiens | transient receptor potential cation channel subfamily M member 1(TRPM1) |
| TRPM1 | BU743036 | Homo sapiens | transient receptor potential cation channel subfamily M member 1(TRPM1) |
| TRPM1 | BU741333 | Homo sapiens | transient receptor potential cation channel subfamily M member 1(TRPM1) |
| ADGRG2 | BC063315 | Homo sapiens | adhesion G protein-coupled receptor G2(ADGRG2) |
| ADGRG2 | AF539455 | Homo sapiens | adhesion G protein-coupled receptor G2(ADGRG2) |
| ADGRG2 | AF539456 | Homo sapiens | adhesion G protein-coupled receptor G2(ADGRG2) |
| ADGRG2 | NM_001079860 | Homo sapiens | adhesion G protein-coupled receptor G2(ADGRG2) |
| ADGRG2 | XM_006724455 | Homo sapiens | adhesion G protein-coupled receptor G2(ADGRG2) |
| ADGRG2 | NM_001184837 | Homo sapiens | adhesion G protein-coupled receptor G2(ADGRG2) |
| ADGRG2 | AF538954 | Homo sapiens | adhesion G protein-coupled receptor G2(ADGRG2) |
| ADGRG2 | AK291012 | Homo sapiens | adhesion G protein-coupled receptor G2(ADGRG2) |
| ADGRG2 | AY148343 | Homo sapiens | adhesion G protein-coupled receptor G2(ADGRG2) |
| ADGRG2 | BC099901 | Homo sapiens | adhesion G protein-coupled receptor G2(ADGRG2) |
| ADGRG2 | BC113979 | Homo sapiens | adhesion G protein-coupled receptor G2(ADGRG2) |
| ADGRG2 | BC113978 | Homo sapiens | adhesion G protein-coupled receptor G2(ADGRG2) |
| ADGRG2 | XM_011545434 | Homo sapiens | adhesion G protein-coupled receptor G2(ADGRG2) |
| ADGRG2 | XM_011545435 | Homo sapiens | adhesion G protein-coupled receptor G2(ADGRG2) |
| ADGRG2 | NM_001184833 | Homo sapiens | adhesion G protein-coupled receptor G2(ADGRG2) |
| ADGRG2 | NM_001184834 | Homo sapiens | adhesion G protein-coupled receptor G2(ADGRG2) |
| ADGRG2 | NM_005756 | Homo sapiens | adhesion G protein-coupled receptor G2(ADGRG2) |
| ADGRG2 | NM_001184835 | Homo sapiens | adhesion G protein-coupled receptor G2(ADGRG2) |
| ADGRG2 | NM_001184836 | Homo sapiens | adhesion G protein-coupled receptor G2(ADGRG2) |
| ADGRG2 | AK309439 | Homo sapiens | adhesion G protein-coupled receptor G2(ADGRG2) |
| ADGRG2 | NM_001079858 | Homo sapiens | adhesion G protein-coupled receptor G2(ADGRG2) |
| ADGRG2 | NM_001079859 | Homo sapiens | adhesion G protein-coupled receptor G2(ADGRG2) |
| ADGRG2 | AY143364 | Homo sapiens | adhesion G protein-coupled receptor G2(ADGRG2) |
| ADGRG2 | AY143366 | Homo sapiens | adhesion G protein-coupled receptor G2(ADGRG2) |
| ADGRG2 | AY143365 | Homo sapiens | adhesion G protein-coupled receptor G2(ADGRG2) |
| ADGRG2 | AY143367 | Homo sapiens | adhesion G protein-coupled receptor G2(ADGRG2) |
| ADGRG2 | X81892 | Homo sapiens | adhesion G protein-coupled receptor G2(ADGRG2) |
| ADGRG2 | AK090402 | Homo sapiens | adhesion G protein-coupled receptor G2(ADGRG2) |
| GALR1 | AY541036 | Homo sapiens | galanin receptor 1(GALR1) |
| GALR1 | NM_001480 | Homo sapiens | galanin receptor 1(GALR1) |
| GALR1 | BC095530 | Homo sapiens | galanin receptor 1(GALR1) |
| GALR1 | U23854 | Homo sapiens | galanin receptor 1(GALR1) |
| GALR1 | XM_017025691 | Homo sapiens | galanin receptor 1(GALR1) |
| GALR1 | L34339 | Homo sapiens | galanin receptor 1(GALR1) |
| GALR1 | U53511 | Homo sapiens | galanin receptor 1(GALR1) |
| MBP | XM_011526009 | Homo sapiens | myelin basic protein(MBP) |
| MBP | BC030093 | Homo sapiens | myelin basic protein(MBP) |
| MBP | NM_001025100 | Homo sapiens | myelin basic protein(MBP) |
| MBP | CR627018 | Homo sapiens | myelin basic protein(MBP) |
| MBP | BC101771 | Homo sapiens | myelin basic protein(MBP) |
| MBP | BC101773 | Homo sapiens | myelin basic protein(MBP) |
| MBP | CR541919 | Homo sapiens | myelin basic protein(MBP) |
| MBP | AK094611 | Homo sapiens | myelin basic protein(MBP) |
| MBP | AK128788 | Homo sapiens | myelin basic protein(MBP) |
| MBP | BC068550 | Homo sapiens | myelin basic protein(MBP) |
| MBP | AK314553 | Homo sapiens | myelin basic protein(MBP) |
| MBP | CB156561 | Homo sapiens | myelin basic protein(MBP) |
| MBP | BU430656 | Homo sapiens | myelin basic protein(MBP) |
| MBP | NM_001025101 | Homo sapiens | myelin basic protein(MBP) |
| MBP | BC065248 | Homo sapiens | myelin basic protein(MBP) |
| MBP | M30515 | Homo sapiens | myelin basic protein(MBP) |
| MBP | AK126858 | Homo sapiens | myelin basic protein(MBP) |
| MBP | CR536534 | Homo sapiens | myelin basic protein(MBP) |
| MBP | BG766942 | Homo sapiens | myelin basic protein(MBP) |
| MBP | N20370 | Homo sapiens | myelin basic protein(MBP) |
| MBP | M20009 | Homo sapiens | myelin basic protein(MBP) |
| MBP | AK074315 | Homo sapiens | myelin basic protein(MBP) |
| MBP | BC008749 | Homo sapiens | myelin basic protein(MBP) |
| MBP | AK124830 | Homo sapiens | myelin basic protein(MBP) |
| MBP | AK293922 | Homo sapiens | myelin basic protein(MBP) |
| MBP | M30516 | Homo sapiens | myelin basic protein(MBP) |
| MBP | M30047 | Homo sapiens | myelin basic protein(MBP) |
| MBP | AB208986 | Homo sapiens | myelin basic protein(MBP) |
| MBP | AK289893 | Homo sapiens | myelin basic protein(MBP) |
| MBP | AK095121 | Homo sapiens | myelin basic protein(MBP) |
| MBP | NM_001025081 | Homo sapiens | myelin basic protein(MBP) |
| MBP | BC080654 | Homo sapiens | myelin basic protein(MBP) |
| MBP | XM_017025779 | Homo sapiens | myelin basic protein(MBP) |
| MBP | XM_017025778 | Homo sapiens | myelin basic protein(MBP) |
| MBP | BC130034 | Homo sapiens | myelin basic protein(MBP) |
| MBP | AK093588 | Homo sapiens | myelin basic protein(MBP) |
| MBP | NM_002385 | Homo sapiens | myelin basic protein(MBP) |
| MBP | AK098513 | Homo sapiens | myelin basic protein(MBP) |
| MBP | AK123433 | Homo sapiens | myelin basic protein(MBP) |
| MBP | L18865 | Homo sapiens | myelin basic protein(MBP) |
| MBP | NM_001025094 | Homo sapiens | myelin basic protein(MBP) |
| MBP | XR_001753201 | Homo sapiens | myelin basic protein(MBP) |
| MBP | XR_001753202 | Homo sapiens | myelin basic protein(MBP) |
| MBP | AK296492 | Homo sapiens | myelin basic protein(MBP) |
| MBP | NM_001025098 | Homo sapiens | myelin basic protein(MBP) |
| MBP | BC143348 | Homo sapiens | myelin basic protein(MBP) |
| MBP | M13577 | Homo sapiens | myelin basic protein(MBP) |
| MBP | XM_017025780 | Homo sapiens | myelin basic protein(MBP) |
| MBP | NM_001025092 | Homo sapiens | myelin basic protein(MBP) |
| MBP | CB153535 | Homo sapiens | myelin basic protein(MBP) |
| MBP | NM_001025090 | Homo sapiens | myelin basic protein(MBP) |
| MBP | AK098402 | Homo sapiens | myelin basic protein(MBP) |
| MBP | BM977768 | Homo sapiens | myelin basic protein(MBP) |
| MBP | AK128770 | Homo sapiens | myelin basic protein(MBP) |
| MBP | AK122594 | Homo sapiens | myelin basic protein(MBP) |
| MBP | BC143350 | Homo sapiens | myelin basic protein(MBP) |
| OTX2 | NM_001270524 | Homo sapiens | orthodenticle homeobox 2(OTX2) |
| OTX2 | BU176852 | Homo sapiens | orthodenticle homeobox 2(OTX2) |
| OTX2 | DQ890875 | Homo sapiens | orthodenticle homeobox 2(OTX2) |
| OTX2 | NM_001270523 | Homo sapiens | orthodenticle homeobox 2(OTX2) |
| OTX2 | BE781530 | Homo sapiens | orthodenticle homeobox 2(OTX2) |
| OTX2 | DB294481 | Homo sapiens | orthodenticle homeobox 2(OTX2) |
| OTX2 | NM_001270525 | Homo sapiens | orthodenticle homeobox 2(OTX2) |
| OTX2 | NM_021728 | Homo sapiens | orthodenticle homeobox 2(OTX2) |
| OTX2 | NM_172337 | Homo sapiens | orthodenticle homeobox 2(OTX2) |
| OTX2 | EU176492 | Homo sapiens | orthodenticle homeobox 2(OTX2) |
| OTX2 | CV812961 | Homo sapiens | orthodenticle homeobox 2(OTX2) |
| OTX2 | AB593056 | Homo sapiens | orthodenticle homeobox 2(OTX2) |
| OTX2 | AK314271 | Homo sapiens | orthodenticle homeobox 2(OTX2) |
| OTX2 | NR_073034 | Homo sapiens | orthodenticle homeobox 2(OTX2) |
| OTX2 | NR_073036 | Homo sapiens | orthodenticle homeobox 2(OTX2) |
| OTX2 | BC032579 | Homo sapiens | orthodenticle homeobox 2(OTX2) |
| OTX2 | AF093138 | Homo sapiens | orthodenticle homeobox 2(OTX2) |
| OTX2 | AB593058 | Homo sapiens | orthodenticle homeobox 2(OTX2) |
| OTX2 | AB593057 | Homo sapiens | orthodenticle homeobox 2(OTX2) |
| PIP5K1B | CB988216 | Homo sapiens | phosphatidylinositol-4-phosphate 5-kinase type 1 beta(PIP5K1B) |
| PIP5K1B | NM_001031687 | Homo sapiens | phosphatidylinositol-4-phosphate 5-kinase type 1 beta(PIP5K1B) |
| PIP5K1B | DQ890874 | Homo sapiens | phosphatidylinositol-4-phosphate 5-kinase type 1 beta(PIP5K1B) |
| PIP5K1B | XM_006717300 | Homo sapiens | phosphatidylinositol-4-phosphate 5-kinase type 1 beta(PIP5K1B) |
| PIP5K1B | XM_006717301 | Homo sapiens | phosphatidylinositol-4-phosphate 5-kinase type 1 beta(PIP5K1B) |
| PIP5K1B | XM_011519082 | Homo sapiens | phosphatidylinositol-4-phosphate 5-kinase type 1 beta(PIP5K1B) |
| PIP5K1B | NM_003558 | Homo sapiens | phosphatidylinositol-4-phosphate 5-kinase type 1 beta(PIP5K1B) |
| PIP5K1B | U78579 | Homo sapiens | phosphatidylinositol-4-phosphate 5-kinase type 1 beta(PIP5K1B) |
| PIP5K1B | XM_011519084 | Homo sapiens | phosphatidylinositol-4-phosphate 5-kinase type 1 beta(PIP5K1B) |
| PIP5K1B | XM_005252262 | Homo sapiens | phosphatidylinositol-4-phosphate 5-kinase type 1 beta(PIP5K1B) |
| PIP5K1B | XM_011519083 | Homo sapiens | phosphatidylinositol-4-phosphate 5-kinase type 1 beta(PIP5K1B) |
| PIP5K1B | XM_005252261 | Homo sapiens | phosphatidylinositol-4-phosphate 5-kinase type 1 beta(PIP5K1B) |
| PIP5K1B | NM_001278253 | Homo sapiens | phosphatidylinositol-4-phosphate 5-kinase type 1 beta(PIP5K1B) |
| PIP5K1B | BC030587 | Homo sapiens | phosphatidylinositol-4-phosphate 5-kinase type 1 beta(PIP5K1B) |
| PIP5K1B | DQ894030 | Homo sapiens | phosphatidylinositol-4-phosphate 5-kinase type 1 beta(PIP5K1B) |
| PIP5K1B | XM_017015190 | Homo sapiens | phosphatidylinositol-4-phosphate 5-kinase type 1 beta(PIP5K1B) |
| PIP5K1B | U78581 | Homo sapiens | phosphatidylinositol-4-phosphate 5-kinase type 1 beta(PIP5K1B) |
| PIP5K1B | XM_017015191 | Homo sapiens | phosphatidylinositol-4-phosphate 5-kinase type 1 beta(PIP5K1B) |
| PIP5K1B | U78580 | Homo sapiens | phosphatidylinositol-4-phosphate 5-kinase type 1 beta(PIP5K1B) |
| PIP5K1B | AK292734 | Homo sapiens | phosphatidylinositol-4-phosphate 5-kinase type 1 beta(PIP5K1B) |
| PIP5K1B | XM_017015188 | Homo sapiens | phosphatidylinositol-4-phosphate 5-kinase type 1 beta(PIP5K1B) |
| PIP5K1B | AK295587 | Homo sapiens | phosphatidylinositol-4-phosphate 5-kinase type 1 beta(PIP5K1B) |
| PIP5K1B | CK299479 | Homo sapiens | phosphatidylinositol-4-phosphate 5-kinase type 1 beta(PIP5K1B) |
| PIP5K1B | XM_017015189 | Homo sapiens | phosphatidylinositol-4-phosphate 5-kinase type 1 beta(PIP5K1B) |
| OTX1 | NR_130153 | Homo sapiens | orthodenticle homeobox 1(OTX1) |
| OTX1 | DQ896509 | Homo sapiens | orthodenticle homeobox 1(OTX1) |
| OTX1 | BU689444 | Homo sapiens | orthodenticle homeobox 1(OTX1) |
| OTX1 | NM_001199770 | Homo sapiens | orthodenticle homeobox 1(OTX1) |
| OTX1 | AK095680 | Homo sapiens | orthodenticle homeobox 1(OTX1) |
| OTX1 | NM_014562 | Homo sapiens | orthodenticle homeobox 1(OTX1) |
| OTX1 | AK297308 | Homo sapiens | orthodenticle homeobox 1(OTX1) |
| OTX1 | BC007621 | Homo sapiens | orthodenticle homeobox 1(OTX1) |
| OTX1 | BP310240 | Homo sapiens | orthodenticle homeobox 1(OTX1) |
| OTX1 | BM720707 | Homo sapiens | orthodenticle homeobox 1(OTX1) |
| OTX1 | DQ893301 | Homo sapiens | orthodenticle homeobox 1(OTX1) |
| FAM83D | BC001068 | Homo sapiens | family with sequence similarity 83 member D(FAM83D) |
| FAM83D | XM_017028088 | Homo sapiens | family with sequence similarity 83 member D(FAM83D) |
| FAM83D | AK303860 | Homo sapiens | family with sequence similarity 83 member D(FAM83D) |
| FAM83D | NM_030919 | Homo sapiens | family with sequence similarity 83 member D(FAM83D) |
| FAM83D | AK055793 | Homo sapiens | family with sequence similarity 83 member D(FAM83D) |
| FAM83D | AL832274 | Homo sapiens | family with sequence similarity 83 member D(FAM83D) |
| FAM83D | BC080188 | Homo sapiens | family with sequence similarity 83 member D(FAM83D) |
| FAM83D | AK095660 | Homo sapiens | family with sequence similarity 83 member D(FAM83D) |
| FAM83D | BC006553 | Homo sapiens | family with sequence similarity 83 member D(FAM83D) |
| FAM83D | BC063661 | Homo sapiens | family with sequence similarity 83 member D(FAM83D) |
| FAM83D | BC053683 | Homo sapiens | family with sequence similarity 83 member D(FAM83D) |
| TRPM3 | XM_011519035 | Homo sapiens | transient receptor potential cation channel subfamily M member 3(TRPM3) |
| TRPM3 | XM_011519037 | Homo sapiens | transient receptor potential cation channel subfamily M member 3(TRPM3) |
| TRPM3 | XM_011519036 | Homo sapiens | transient receptor potential cation channel subfamily M member 3(TRPM3) |
| TRPM3 | AL136545 | Homo sapiens | transient receptor potential cation channel subfamily M member 3(TRPM3) |
| TRPM3 | XM_011519039 | Homo sapiens | transient receptor potential cation channel subfamily M member 3(TRPM3) |
| TRPM3 | XM_011519038 | Homo sapiens | transient receptor potential cation channel subfamily M member 3(TRPM3) |
| TRPM3 | BC121821 | Homo sapiens | transient receptor potential cation channel subfamily M member 3(TRPM3) |
| TRPM3 | BC067733 | Homo sapiens | transient receptor potential cation channel subfamily M member 3(TRPM3) |
| TRPM3 | XM_017015141 | Homo sapiens | transient receptor potential cation channel subfamily M member 3(TRPM3) |
| TRPM3 | XM_017015142 | Homo sapiens | transient receptor potential cation channel subfamily M member 3(TRPM3) |
| TRPM3 | BC134414 | Homo sapiens | transient receptor potential cation channel subfamily M member 3(TRPM3) |
| TRPM3 | NM_001007470 | Homo sapiens | transient receptor potential cation channel subfamily M member 3(TRPM3) |
| TRPM3 | NM_001007471 | Homo sapiens | transient receptor potential cation channel subfamily M member 3(TRPM3) |
| TRPM3 | XM_017015147 | Homo sapiens | transient receptor potential cation channel subfamily M member 3(TRPM3) |
| TRPM3 | XM_017015148 | Homo sapiens | transient receptor potential cation channel subfamily M member 3(TRPM3) |
| TRPM3 | AK308682 | Homo sapiens | transient receptor potential cation channel subfamily M member 3(TRPM3) |
| TRPM3 | XM_017015149 | Homo sapiens | transient receptor potential cation channel subfamily M member 3(TRPM3) |
| TRPM3 | XM_017015143 | Homo sapiens | transient receptor potential cation channel subfamily M member 3(TRPM3) |
| TRPM3 | XM_017015144 | Homo sapiens | transient receptor potential cation channel subfamily M member 3(TRPM3) |
| TRPM3 | XM_017015145 | Homo sapiens | transient receptor potential cation channel subfamily M member 3(TRPM3) |
| TRPM3 | XM_017015146 | Homo sapiens | transient receptor potential cation channel subfamily M member 3(TRPM3) |
| TRPM3 | AK225732 | Homo sapiens | transient receptor potential cation channel subfamily M member 3(TRPM3) |
| TRPM3 | XM_011519044 | Homo sapiens | transient receptor potential cation channel subfamily M member 3(TRPM3) |
| TRPM3 | XM_011519043 | Homo sapiens | transient receptor potential cation channel subfamily M member 3(TRPM3) |
| TRPM3 | XM_011519046 | Homo sapiens | transient receptor potential cation channel subfamily M member 3(TRPM3) |
| TRPM3 | XM_011519045 | Homo sapiens | transient receptor potential cation channel subfamily M member 3(TRPM3) |
| TRPM3 | XM_011519047 | Homo sapiens | transient receptor potential cation channel subfamily M member 3(TRPM3) |
| TRPM3 | AB099665 | Homo sapiens | transient receptor potential cation channel subfamily M member 3(TRPM3) |
| TRPM3 | AB099663 | Homo sapiens | transient receptor potential cation channel subfamily M member 3(TRPM3) |
| TRPM3 | AB099664 | Homo sapiens | transient receptor potential cation channel subfamily M member 3(TRPM3) |
| TRPM3 | AB099661 | Homo sapiens | transient receptor potential cation channel subfamily M member 3(TRPM3) |
| TRPM3 | NM_020952 | Homo sapiens | transient receptor potential cation channel subfamily M member 3(TRPM3) |
| TRPM3 | XM_011519040 | Homo sapiens | transient receptor potential cation channel subfamily M member 3(TRPM3) |
| TRPM3 | AB099662 | Homo sapiens | transient receptor potential cation channel subfamily M member 3(TRPM3) |
| TRPM3 | XM_011519042 | Homo sapiens | transient receptor potential cation channel subfamily M member 3(TRPM3) |
| TRPM3 | XM_011519041 | Homo sapiens | transient receptor potential cation channel subfamily M member 3(TRPM3) |
| TRPM3 | AK021788 | Homo sapiens | transient receptor potential cation channel subfamily M member 3(TRPM3) |
| TRPM3 | CD673832 | Homo sapiens | transient receptor potential cation channel subfamily M member 3(TRPM3) |
| TRPM3 | AF325212 | Homo sapiens | transient receptor potential cation channel subfamily M member 3(TRPM3) |
| TRPM3 | AJ505026 | Homo sapiens | transient receptor potential cation channel subfamily M member 3(TRPM3) |
| TRPM3 | AJ505025 | Homo sapiens | transient receptor potential cation channel subfamily M member 3(TRPM3) |
| TRPM3 | BC172350 | Homo sapiens | transient receptor potential cation channel subfamily M member 3(TRPM3) |
| TRPM3 | BM668925 | Homo sapiens | transient receptor potential cation channel subfamily M member 3(TRPM3) |
| TRPM3 | NM_206947 | Homo sapiens | transient receptor potential cation channel subfamily M member 3(TRPM3) |
| TRPM3 | XM_017015161 | Homo sapiens | transient receptor potential cation channel subfamily M member 3(TRPM3) |
| TRPM3 | NM_206948 | Homo sapiens | transient receptor potential cation channel subfamily M member 3(TRPM3) |
| TRPM3 | NM_206944 | Homo sapiens | transient receptor potential cation channel subfamily M member 3(TRPM3) |
| TRPM3 | NM_206945 | Homo sapiens | transient receptor potential cation channel subfamily M member 3(TRPM3) |
| TRPM3 | NM_206946 | Homo sapiens | transient receptor potential cation channel subfamily M member 3(TRPM3) |
| TRPM3 | XM_017015160 | Homo sapiens | transient receptor potential cation channel subfamily M member 3(TRPM3) |
| TRPM3 | AF536752 | Homo sapiens | transient receptor potential cation channel subfamily M member 3(TRPM3) |
| TRPM3 | AF536751 | Homo sapiens | transient receptor potential cation channel subfamily M member 3(TRPM3) |
| TRPM3 | AF536753 | Homo sapiens | transient receptor potential cation channel subfamily M member 3(TRPM3) |
| TRPM3 | BC094699 | Homo sapiens | transient receptor potential cation channel subfamily M member 3(TRPM3) |
| TRPM3 | AF536750 | Homo sapiens | transient receptor potential cation channel subfamily M member 3(TRPM3) |
| TRPM3 | BC142972 | Homo sapiens | transient receptor potential cation channel subfamily M member 3(TRPM3) |
| TRPM3 | NM_024971 | Homo sapiens | transient receptor potential cation channel subfamily M member 3(TRPM3) |
| TRPM3 | BC022454 | Homo sapiens | transient receptor potential cation channel subfamily M member 3(TRPM3) |
| TRPM3 | AB046836 | Homo sapiens | transient receptor potential cation channel subfamily M member 3(TRPM3) |
| TRPM3 | CD104154 | Homo sapiens | transient receptor potential cation channel subfamily M member 3(TRPM3) |
| TRPM3 | KF987075 | Homo sapiens | transient receptor potential cation channel subfamily M member 3(TRPM3) |
| TRPM3 | AF536749 | Homo sapiens | transient receptor potential cation channel subfamily M member 3(TRPM3) |
| TRPM3 | XM_017015150 | Homo sapiens | transient receptor potential cation channel subfamily M member 3(TRPM3) |
| TRPM3 | AF536748 | Homo sapiens | transient receptor potential cation channel subfamily M member 3(TRPM3) |
| TRPM3 | XM_017015151 | Homo sapiens | transient receptor potential cation channel subfamily M member 3(TRPM3) |
| TRPM3 | XM_017015152 | Homo sapiens | transient receptor potential cation channel subfamily M member 3(TRPM3) |
| TRPM3 | XM_017015153 | Homo sapiens | transient receptor potential cation channel subfamily M member 3(TRPM3) |
| TRPM3 | XM_017015158 | Homo sapiens | transient receptor potential cation channel subfamily M member 3(TRPM3) |
| TRPM3 | XM_017015159 | Homo sapiens | transient receptor potential cation channel subfamily M member 3(TRPM3) |
| TRPM3 | XM_017015154 | Homo sapiens | transient receptor potential cation channel subfamily M member 3(TRPM3) |
| TRPM3 | XM_017015155 | Homo sapiens | transient receptor potential cation channel subfamily M member 3(TRPM3) |
| TRPM3 | XM_017015156 | Homo sapiens | transient receptor potential cation channel subfamily M member 3(TRPM3) |
| TRPM3 | XM_017015157 | Homo sapiens | transient receptor potential cation channel subfamily M member 3(TRPM3) |
| HSPA9 | AU130219 | Homo sapiens | heat shock protein family A (Hsp70) member 9(HSPA9) |
| HSPA9 | DQ185038 | Homo sapiens | heat shock protein family A (Hsp70) member 9(HSPA9) |
| HSPA9 | AK225488 | Homo sapiens | heat shock protein family A (Hsp70) member 9(HSPA9) |
| HSPA9 | AK297795 | Homo sapiens | heat shock protein family A (Hsp70) member 9(HSPA9) |
| HSPA9 | AK293990 | Homo sapiens | heat shock protein family A (Hsp70) member 9(HSPA9) |
| HSPA9 | DQ480334 | Homo sapiens | heat shock protein family A (Hsp70) member 9(HSPA9) |
| HSPA9 | BC024034 | Homo sapiens | heat shock protein family A (Hsp70) member 9(HSPA9) |
| HSPA9 | FJ224291 | Homo sapiens | heat shock protein family A (Hsp70) member 9(HSPA9) |
| HSPA9 | AK023317 | Homo sapiens | heat shock protein family A (Hsp70) member 9(HSPA9) |
| HSPA9 | BC000478 | Homo sapiens | heat shock protein family A (Hsp70) member 9(HSPA9) |
| HSPA9 | BX426279 | Homo sapiens | heat shock protein family A (Hsp70) member 9(HSPA9) |
| HSPA9 | AK315177 | Homo sapiens | heat shock protein family A (Hsp70) member 9(HSPA9) |
| HSPA9 | BC030634 | Homo sapiens | heat shock protein family A (Hsp70) member 9(HSPA9) |
| HSPA9 | NM_004134 | Homo sapiens | heat shock protein family A (Hsp70) member 9(HSPA9) |
| HSPA9 | AK222758 | Homo sapiens | heat shock protein family A (Hsp70) member 9(HSPA9) |
| HSPA9 | EU446981 | Homo sapiens | heat shock protein family A (Hsp70) member 9(HSPA9) |
| HSPA9 | JF432370 | Homo sapiens | heat shock protein family A (Hsp70) member 9(HSPA9) |
| HSPA9 | L15189 | Homo sapiens | heat shock protein family A (Hsp70) member 9(HSPA9) |
| HSPA9 | DC356852 | Homo sapiens | heat shock protein family A (Hsp70) member 9(HSPA9) |
| HSPA9 | L11066 | Homo sapiens | heat shock protein family A (Hsp70) member 9(HSPA9) |
| HSPA9 | AK297897 | Homo sapiens | heat shock protein family A (Hsp70) member 9(HSPA9) |
| TFAP2B | AK291172 | Homo sapiens | transcription factor AP-2 beta(TFAP2B) |
| TFAP2B | AK313749 | Homo sapiens | transcription factor AP-2 beta(TFAP2B) |
| TFAP2B | BC037225 | Homo sapiens | transcription factor AP-2 beta(TFAP2B) |
| TFAP2B | DQ891443 | Homo sapiens | transcription factor AP-2 beta(TFAP2B) |
| TFAP2B | XM_017011235 | Homo sapiens | transcription factor AP-2 beta(TFAP2B) |
| TFAP2B | BU738725 | Homo sapiens | transcription factor AP-2 beta(TFAP2B) |
| TFAP2B | NM_003221 | Homo sapiens | transcription factor AP-2 beta(TFAP2B) |
| TFAP2B | X95694 | Homo sapiens | transcription factor AP-2 beta(TFAP2B) |
| TFAP2B | DQ894621 | Homo sapiens | transcription factor AP-2 beta(TFAP2B) |
| TFAP2B | XM_017011233 | Homo sapiens | transcription factor AP-2 beta(TFAP2B) |
| TFAP2B | AU141084 | Homo sapiens | transcription factor AP-2 beta(TFAP2B) |
| TFAP2B | XM_011514837 | Homo sapiens | transcription factor AP-2 beta(TFAP2B) |
| TFAP2B | XM_017011234 | Homo sapiens | transcription factor AP-2 beta(TFAP2B) |
| FZD3 | AK291480 | Homo sapiens | frizzled class receptor 3(FZD3) |
| FZD3 | BU676090 | Homo sapiens | frizzled class receptor 3(FZD3) |
| FZD3 | XM_017013844 | Homo sapiens | frizzled class receptor 3(FZD3) |
| FZD3 | XM_017013843 | Homo sapiens | frizzled class receptor 3(FZD3) |
| FZD3 | XM_017013842 | Homo sapiens | frizzled class receptor 3(FZD3) |
| FZD3 | NM_017412 | Homo sapiens | frizzled class receptor 3(FZD3) |
| FZD3 | XR_001745597 | Homo sapiens | frizzled class receptor 3(FZD3) |
| FZD3 | BC042009 | Homo sapiens | frizzled class receptor 3(FZD3) |
| FZD3 | XR_949476 | Homo sapiens | frizzled class receptor 3(FZD3) |
| FZD3 | BC172481 | Homo sapiens | frizzled class receptor 3(FZD3) |
| FZD3 | BC156293 | Homo sapiens | frizzled class receptor 3(FZD3) |
| FZD3 | AY005130 | Homo sapiens | frizzled class receptor 3(FZD3) |
| FZD3 | NM_145866 | Homo sapiens | frizzled class receptor 3(FZD3) |
| FZD3 | AB039723 | Homo sapiens | frizzled class receptor 3(FZD3) |
| FZD3 | XM_017013841 | Homo sapiens | frizzled class receptor 3(FZD3) |
| FZD3 | BC034278 | Homo sapiens | frizzled class receptor 3(FZD3) |
| FZD3 | AJ272427 | Homo sapiens | frizzled class receptor 3(FZD3) |
| FZD3 | DA250029 | Homo sapiens | frizzled class receptor 3(FZD3) |
| DNAH11 | BQ365981 | Homo sapiens | dynein axonemal heavy chain 11(DNAH11) |
| DNAH11 | JQ247523 | Homo sapiens | dynein axonemal heavy chain 11(DNAH11) |
| DNAH11 | AK095018 | Homo sapiens | dynein axonemal heavy chain 11(DNAH11) |
| DNAH11 | AL529367 | Homo sapiens | dynein axonemal heavy chain 11(DNAH11) |
| DNAH11 | CA424782 | Homo sapiens | dynein axonemal heavy chain 11(DNAH11) |
| DNAH11 | JQ247524 | Homo sapiens | dynein axonemal heavy chain 11(DNAH11) |
| DNAH11 | AJ132087 | Homo sapiens | dynein axonemal heavy chain 11(DNAH11) |
| DNAH11 | AK054657 | Homo sapiens | dynein axonemal heavy chain 11(DNAH11) |
| DNAH11 | NM_001277115 | Homo sapiens | dynein axonemal heavy chain 11(DNAH11) |
| DNAH11 | AJ320497 | Homo sapiens | dynein axonemal heavy chain 11(DNAH11) |
| DNAH11 | AI743846 | Homo sapiens | dynein axonemal heavy chain 11(DNAH11) |
| DNAH11 | BF369731 | Homo sapiens | dynein axonemal heavy chain 11(DNAH11) |
| PLK1 | AB209179 | Homo sapiens | polo like kinase 1(PLK1) |
| PLK1 | AK303263 | Homo sapiens | polo like kinase 1(PLK1) |
| PLK1 | AK308276 | Homo sapiens | polo like kinase 1(PLK1) |
| PLK1 | X75932 | Homo sapiens | polo like kinase 1(PLK1) |
| PLK1 | BC014846 | Homo sapiens | polo like kinase 1(PLK1) |
| PLK1 | X73458 | Homo sapiens | polo like kinase 1(PLK1) |
| PLK1 | DC300189 | Homo sapiens | polo like kinase 1(PLK1) |
| PLK1 | AB084459 | Homo sapiens | polo like kinase 1(PLK1) |
| PLK1 | NM_005030 | Homo sapiens | polo like kinase 1(PLK1) |
| PLK1 | BC003002 | Homo sapiens | polo like kinase 1(PLK1) |
| PLK1 | BC002369 | Homo sapiens | polo like kinase 1(PLK1) |
| PLK1 | L19559 | Homo sapiens | polo like kinase 1(PLK1) |
| PLK1 | U01038 | Homo sapiens | polo like kinase 1(PLK1) |
| PLK1 | DQ891045 | Homo sapiens | polo like kinase 1(PLK1) |
| PLK1 | AK313227 | Homo sapiens | polo like kinase 1(PLK1) |
| PLK1 | DQ894224 | Homo sapiens | polo like kinase 1(PLK1) |
| FN1 | AF130095 | Homo sapiens | fibronectin 1(FN1) |
| FN1 | AJ849445 | Homo sapiens | fibronectin 1(FN1) |
| FN1 | BX641150 | Homo sapiens | fibronectin 1(FN1) |
| FN1 | CA422234 | Homo sapiens | fibronectin 1(FN1) |
| FN1 | NM_054034 | Homo sapiens | fibronectin 1(FN1) |
| FN1 | AF312399 | Homo sapiens | fibronectin 1(FN1) |
| FN1 | BX538018 | Homo sapiens | fibronectin 1(FN1) |
| FN1 | U41724 | Homo sapiens | fibronectin 1(FN1) |
| FN1 | BX538017 | Homo sapiens | fibronectin 1(FN1) |
| FN1 | NM_001306129 | Homo sapiens | fibronectin 1(FN1) |
| FN1 | BC016875 | Homo sapiens | fibronectin 1(FN1) |
| FN1 | CR749281 | Homo sapiens | fibronectin 1(FN1) |
| FN1 | EU831936 | Homo sapiens | fibronectin 1(FN1) |
| FN1 | NM_001306132 | Homo sapiens | fibronectin 1(FN1) |
| FN1 | NM_001306130 | Homo sapiens | fibronectin 1(FN1) |
| FN1 | XM_005246408 | Homo sapiens | fibronectin 1(FN1) |
| FN1 | NM_001306131 | Homo sapiens | fibronectin 1(FN1) |
| FN1 | XM_005246407 | Homo sapiens | fibronectin 1(FN1) |
| FN1 | NM_002026 | Homo sapiens | fibronectin 1(FN1) |
| FN1 | XM_005246406 | Homo sapiens | fibronectin 1(FN1) |
| FN1 | XM_005246405 | Homo sapiens | fibronectin 1(FN1) |
| FN1 | XM_005246409 | Homo sapiens | fibronectin 1(FN1) |
| FN1 | BX640875 | Homo sapiens | fibronectin 1(FN1) |
| FN1 | CR749317 | Homo sapiens | fibronectin 1(FN1) |
| FN1 | BX640638 | Homo sapiens | fibronectin 1(FN1) |
| FN1 | CR749316 | Homo sapiens | fibronectin 1(FN1) |
| FN1 | XM_005246404 | Homo sapiens | fibronectin 1(FN1) |
| FN1 | XM_005246403 | Homo sapiens | fibronectin 1(FN1) |
| FN1 | BX640999 | Homo sapiens | fibronectin 1(FN1) |
| FN1 | XM_005246402 | Homo sapiens | fibronectin 1(FN1) |
| FN1 | XM_005246401 | Homo sapiens | fibronectin 1(FN1) |
| FN1 | M27590 | Homo sapiens | fibronectin 1(FN1) |
| FN1 | XM_005246416 | Homo sapiens | fibronectin 1(FN1) |
| FN1 | AK300216 | Homo sapiens | fibronectin 1(FN1) |
| FN1 | AK300458 | Homo sapiens | fibronectin 1(FN1) |
| FN1 | XM_005246411 | Homo sapiens | fibronectin 1(FN1) |
| FN1 | XM_005246410 | Homo sapiens | fibronectin 1(FN1) |
| FN1 | AK316350 | Homo sapiens | fibronectin 1(FN1) |
| FN1 | AL832202 | Homo sapiens | fibronectin 1(FN1) |
| FN1 | XM_005246414 | Homo sapiens | fibronectin 1(FN1) |
| FN1 | CR749666 | Homo sapiens | fibronectin 1(FN1) |
| FN1 | XM_005246412 | Homo sapiens | fibronectin 1(FN1) |
| FN1 | M27589 | Homo sapiens | fibronectin 1(FN1) |
| FN1 | U42455 | Homo sapiens | fibronectin 1(FN1) |
| FN1 | BC117176 | Homo sapiens | fibronectin 1(FN1) |
| FN1 | BX537590 | Homo sapiens | fibronectin 1(FN1) |
| FN1 | U42456 | Homo sapiens | fibronectin 1(FN1) |
| FN1 | U42457 | Homo sapiens | fibronectin 1(FN1) |
| FN1 | U42458 | Homo sapiens | fibronectin 1(FN1) |
| FN1 | BX538045 | Homo sapiens | fibronectin 1(FN1) |
| FN1 | BX640608 | Homo sapiens | fibronectin 1(FN1) |
| FN1 | BX640731 | Homo sapiens | fibronectin 1(FN1) |
| FN1 | AB191261 | Homo sapiens | fibronectin 1(FN1) |
| FN1 | M10905 | Homo sapiens | fibronectin 1(FN1) |
| FN1 | BQ005645 | Homo sapiens | fibronectin 1(FN1) |
| FN1 | BC078656 | Homo sapiens | fibronectin 1(FN1) |
| FN1 | X02761 | Homo sapiens | fibronectin 1(FN1) |
| FN1 | EF550130 | Homo sapiens | fibronectin 1(FN1) |
| FN1 | BT006856 | Homo sapiens | fibronectin 1(FN1) |
| FN1 | AI033037 | Homo sapiens | fibronectin 1(FN1) |
| FN1 | CR936623 | Homo sapiens | fibronectin 1(FN1) |
| FN1 | XM_005246399 | Homo sapiens | fibronectin 1(FN1) |
| FN1 | XM_005246398 | Homo sapiens | fibronectin 1(FN1) |
| FN1 | XM_005246397 | Homo sapiens | fibronectin 1(FN1) |
| FN1 | U42592 | Homo sapiens | fibronectin 1(FN1) |
| FN1 | U42593 | Homo sapiens | fibronectin 1(FN1) |
| FN1 | U42594 | Homo sapiens | fibronectin 1(FN1) |
| FN1 | AK297733 | Homo sapiens | fibronectin 1(FN1) |
| FN1 | BC143763 | Homo sapiens | fibronectin 1(FN1) |
| FN1 | EF550135 | Homo sapiens | fibronectin 1(FN1) |
| FN1 | EF550134 | Homo sapiens | fibronectin 1(FN1) |
| FN1 | EF550133 | Homo sapiens | fibronectin 1(FN1) |
| FN1 | EF550132 | Homo sapiens | fibronectin 1(FN1) |
| FN1 | EF550131 | Homo sapiens | fibronectin 1(FN1) |
| FN1 | AL832771 | Homo sapiens | fibronectin 1(FN1) |
| FN1 | NM_212476 | Homo sapiens | fibronectin 1(FN1) |
| FN1 | NM_212478 | Homo sapiens | fibronectin 1(FN1) |
| FN1 | AJ320527 | Homo sapiens | fibronectin 1(FN1) |
| FN1 | U42404 | Homo sapiens | fibronectin 1(FN1) |
| FN1 | AJ320526 | Homo sapiens | fibronectin 1(FN1) |
| FN1 | BC143754 | Homo sapiens | fibronectin 1(FN1) |
| FN1 | AJ320525 | Homo sapiens | fibronectin 1(FN1) |
| FN1 | DA851446 | Homo sapiens | fibronectin 1(FN1) |
| FN1 | AK094153 | Homo sapiens | fibronectin 1(FN1) |
| FN1 | BX640920 | Homo sapiens | fibronectin 1(FN1) |
| FN1 | BX640803 | Homo sapiens | fibronectin 1(FN1) |
| FN1 | BX640802 | Homo sapiens | fibronectin 1(FN1) |
| FN1 | U60068 | Homo sapiens | fibronectin 1(FN1) |
| FN1 | U60067 | Homo sapiens | fibronectin 1(FN1) |
| FN1 | BC100030 | Homo sapiens | fibronectin 1(FN1) |
| FN1 | NM_212482 | Homo sapiens | fibronectin 1(FN1) |
| FN1 | EU832031 | Homo sapiens | fibronectin 1(FN1) |
| FN1 | AB209840 | Homo sapiens | fibronectin 1(FN1) |
| FN1 | BX649182 | Homo sapiens | fibronectin 1(FN1) |
| FN1 | AK300246 | Homo sapiens | fibronectin 1(FN1) |
| FN1 | AJ276395 | Homo sapiens | fibronectin 1(FN1) |
| FN1 | XM_017003693 | Homo sapiens | fibronectin 1(FN1) |
| FN1 | XM_017003692 | Homo sapiens | fibronectin 1(FN1) |
| FN1 | AB209287 | Homo sapiens | fibronectin 1(FN1) |
| FN1 | XM_017003695 | Homo sapiens | fibronectin 1(FN1) |
| FN1 | XM_017003694 | Homo sapiens | fibronectin 1(FN1) |
| FN1 | AJ535086 | Homo sapiens | fibronectin 1(FN1) |
| FN1 | BC005858 | Homo sapiens | fibronectin 1(FN1) |
| FN1 | AK026737 | Homo sapiens | fibronectin 1(FN1) |
| FN1 | NM_212474 | Homo sapiens | fibronectin 1(FN1) |
| FN1 | U41850 | Homo sapiens | fibronectin 1(FN1) |
| FN1 | NM_212475 | Homo sapiens | fibronectin 1(FN1) |
| TYR | J03581 | Homo sapiens | tyrosinase(TYR) |
| TYR | AB775901 | Homo sapiens | tyrosinase(TYR) |
| TYR | XM_011542970 | Homo sapiens | tyrosinase(TYR) |
| TYR | AB775899 | Homo sapiens | tyrosinase(TYR) |
| TYR | M27160 | Homo sapiens | tyrosinase(TYR) |
| TYR | AB775900 | Homo sapiens | tyrosinase(TYR) |
| TYR | M74314 | Homo sapiens | tyrosinase(TYR) |
| TYR | BC027179 | Homo sapiens | tyrosinase(TYR) |
| TYR | BU736025 | Homo sapiens | tyrosinase(TYR) |
| TYR | S66645 | Homo sapiens | tyrosinase(TYR) |
| TYR | NM_000372 | Homo sapiens | tyrosinase(TYR) |
| TYR | U01873 | Homo sapiens | tyrosinase(TYR) |
| TYR | Y00819 | Homo sapiens | tyrosinase(TYR) |
| LRP1B | AF176832 | Homo sapiens | LDL receptor related protein 1B(LRP1B) |
| LRP1B | AK054663 | Homo sapiens | LDL receptor related protein 1B(LRP1B) |
| LRP1B | NM_018557 | Homo sapiens | LDL receptor related protein 1B(LRP1B) |
| LRP1B | AB209707 | Homo sapiens | LDL receptor related protein 1B(LRP1B) |
| LRP1B | XR_001738778 | Homo sapiens | LDL receptor related protein 1B(LRP1B) |
| LRP1B | XM_017004341 | Homo sapiens | LDL receptor related protein 1B(LRP1B) |
| LRP1B | XM_017004342 | Homo sapiens | LDL receptor related protein 1B(LRP1B) |
| PHOX2B | NM_003924 | Homo sapiens | paired like homeobox 2b(PHOX2B) |
| PHOX2B | D82344 | Homo sapiens | paired like homeobox 2b(PHOX2B) |
| PHOX2B | AI266171 | Homo sapiens | paired like homeobox 2b(PHOX2B) |
| PHOX2B | BC017199 | Homo sapiens | paired like homeobox 2b(PHOX2B) |
| GFAP | BC041765 | Homo sapiens | glial fibrillary acidic protein(GFAP) |
| GFAP | DA254392 | Homo sapiens | glial fibrillary acidic protein(GFAP) |
| GFAP | BC013596 | Homo sapiens | glial fibrillary acidic protein(GFAP) |
| GFAP | NM_001242376 | Homo sapiens | glial fibrillary acidic protein(GFAP) |
| GFAP | AL133013 | Homo sapiens | glial fibrillary acidic protein(GFAP) |
| GFAP | BC012228 | Homo sapiens | glial fibrillary acidic protein(GFAP) |
| GFAP | AK098758 | Homo sapiens | glial fibrillary acidic protein(GFAP) |
| GFAP | AK303728 | Homo sapiens | glial fibrillary acidic protein(GFAP) |
| GFAP | JF432461 | Homo sapiens | glial fibrillary acidic protein(GFAP) |
| GFAP | M26638 | Homo sapiens | glial fibrillary acidic protein(GFAP) |
| GFAP | NM_001131019 | Homo sapiens | glial fibrillary acidic protein(GFAP) |
| GFAP | AK295734 | Homo sapiens | glial fibrillary acidic protein(GFAP) |
| GFAP | BC127871 | Homo sapiens | glial fibrillary acidic protein(GFAP) |
| GFAP | AK124465 | Homo sapiens | glial fibrillary acidic protein(GFAP) |
| GFAP | BC062609 | Homo sapiens | glial fibrillary acidic protein(GFAP) |
| GFAP | BM931499 | Homo sapiens | glial fibrillary acidic protein(GFAP) |
| GFAP | AK098380 | Homo sapiens | glial fibrillary acidic protein(GFAP) |
| GFAP | AK222683 | Homo sapiens | glial fibrillary acidic protein(GFAP) |
| GFAP | S40719 | Homo sapiens | glial fibrillary acidic protein(GFAP) |
| GFAP | DQ979832 | Homo sapiens | glial fibrillary acidic protein(GFAP) |
| GFAP | XM_017024451 | Homo sapiens | glial fibrillary acidic protein(GFAP) |
| GFAP | J04569 | Homo sapiens | glial fibrillary acidic protein(GFAP) |
| GFAP | U92979 | Homo sapiens | glial fibrillary acidic protein(GFAP) |
| GFAP | BF526347 | Homo sapiens | glial fibrillary acidic protein(GFAP) |
| GFAP | BM687259 | Homo sapiens | glial fibrillary acidic protein(GFAP) |
| GFAP | AF419299 | Homo sapiens | glial fibrillary acidic protein(GFAP) |
| GFAP | BF341765 | Homo sapiens | glial fibrillary acidic protein(GFAP) |
| GFAP | NM_002055 | Homo sapiens | glial fibrillary acidic protein(GFAP) |
| GFAP | AK315398 | Homo sapiens | glial fibrillary acidic protein(GFAP) |
| GFAP | DA315956 | Homo sapiens | glial fibrillary acidic protein(GFAP) |
| GFAP | AJ306447 | Homo sapiens | glial fibrillary acidic protein(GFAP) |
| GFAP | AK128790 | Homo sapiens | glial fibrillary acidic protein(GFAP) |
| GFAP | AK296259 | Homo sapiens | glial fibrillary acidic protein(GFAP) |
| PHOX2A | BQ890461 | Homo sapiens | paired like homeobox 2a(PHOX2A) |
| PHOX2A | AK290645 | Homo sapiens | paired like homeobox 2a(PHOX2A) |
| PHOX2A | NM_005169 | Homo sapiens | paired like homeobox 2a(PHOX2A) |
| PHOX2A | BC041564 | Homo sapiens | paired like homeobox 2a(PHOX2A) |
| PHOX2A | EU446462 | Homo sapiens | paired like homeobox 2a(PHOX2A) |
| GDF7 | BC160118 | Homo sapiens | growth differentiation factor 7(GDF7) |
| GDF7 | BQ182131 | Homo sapiens | growth differentiation factor 7(GDF7) |
| GDF7 | AB158468 | Homo sapiens | growth differentiation factor 7(GDF7) |
| GDF7 | NM_182828 | Homo sapiens | growth differentiation factor 7(GDF7) |
| GDF7 | AF522369 | Homo sapiens | growth differentiation factor 7(GDF7) |
| COL1A1 | AB209597 | Homo sapiens | collagen type I alpha 1 chain(COL1A1) |
| COL1A1 | AK297731 | Homo sapiens | collagen type I alpha 1 chain(COL1A1) |
| COL1A1 | M32798 | Homo sapiens | collagen type I alpha 1 chain(COL1A1) |
| COL1A1 | DQ590066 | Homo sapiens | collagen type I alpha 1 chain(COL1A1) |
| COL1A1 | XM_005257059 | Homo sapiens | collagen type I alpha 1 chain(COL1A1) |
| COL1A1 | XM_005257058 | Homo sapiens | collagen type I alpha 1 chain(COL1A1) |
| COL1A1 | NM_000088 | Homo sapiens | collagen type I alpha 1 chain(COL1A1) |
| COL1A1 | S67495 | Homo sapiens | collagen type I alpha 1 chain(COL1A1) |
| COL1A1 | M36546 | Homo sapiens | collagen type I alpha 1 chain(COL1A1) |
| COL1A1 | JQ236861 | Homo sapiens | collagen type I alpha 1 chain(COL1A1) |
| COL1A1 | Z74615 | Homo sapiens | collagen type I alpha 1 chain(COL1A1) |
| COL1A1 | S64596 | Homo sapiens | collagen type I alpha 1 chain(COL1A1) |
| COL1A1 | EU176569 | Homo sapiens | collagen type I alpha 1 chain(COL1A1) |
| COL1A1 | DQ893571 | Homo sapiens | collagen type I alpha 1 chain(COL1A1) |
| COL1A1 | K01228 | Homo sapiens | collagen type I alpha 1 chain(COL1A1) |
| COL1A1 | XM_011524341 | Homo sapiens | collagen type I alpha 1 chain(COL1A1) |
| COL1A1 | X07884 | Homo sapiens | collagen type I alpha 1 chain(COL1A1) |
| COL1A1 | BC036531 | Homo sapiens | collagen type I alpha 1 chain(COL1A1) |
| COL1A1 | S64717 | Homo sapiens | collagen type I alpha 1 chain(COL1A1) |
| COL1A1 | X06269 | Homo sapiens | collagen type I alpha 1 chain(COL1A1) |
| COL1A2 | AK297786 | Homo sapiens | collagen type I alpha 2 chain(COL1A2) |
| COL1A2 | AK309504 | Homo sapiens | collagen type I alpha 2 chain(COL1A2) |
| COL1A2 | AK300194 | Homo sapiens | collagen type I alpha 2 chain(COL1A2) |
| COL1A2 | NM_000089 | Homo sapiens | collagen type I alpha 2 chain(COL1A2) |
| COL1A2 | AA457209 | Homo sapiens | collagen type I alpha 2 chain(COL1A2) |
| COL1A2 | J03464 | Homo sapiens | collagen type I alpha 2 chain(COL1A2) |
| COL1A2 | AK226074 | Homo sapiens | collagen type I alpha 2 chain(COL1A2) |
| COL1A2 | BC042586 | Homo sapiens | collagen type I alpha 2 chain(COL1A2) |
| COL1A2 | J00114 | Homo sapiens | collagen type I alpha 2 chain(COL1A2) |
| COL1A2 | M22816 | Homo sapiens | collagen type I alpha 2 chain(COL1A2) |
| COL1A2 | X55525 | Homo sapiens | collagen type I alpha 2 chain(COL1A2) |
| COL1A2 | L47668 | Homo sapiens | collagen type I alpha 2 chain(COL1A2) |
| COL1A2 | AL833478 | Homo sapiens | collagen type I alpha 2 chain(COL1A2) |
| COL1A2 | S41099 | Homo sapiens | collagen type I alpha 2 chain(COL1A2) |
| COL1A2 | Z74616 | Homo sapiens | collagen type I alpha 2 chain(COL1A2) |
| COL1A2 | S62614 | Homo sapiens | collagen type I alpha 2 chain(COL1A2) |
| COL1A2 | S96821 | Homo sapiens | collagen type I alpha 2 chain(COL1A2) |
| COL1A2 | V00503 | Homo sapiens | collagen type I alpha 2 chain(COL1A2) |
| COL1A2 | KU177988 | Homo sapiens | collagen type I alpha 2 chain(COL1A2) |
| COL1A2 | Y00724 | Homo sapiens | collagen type I alpha 2 chain(COL1A2) |
| COL1A2 | KU177989 | Homo sapiens | collagen type I alpha 2 chain(COL1A2) |
| COL1A2 | CF125721 | Homo sapiens | collagen type I alpha 2 chain(COL1A2) |
| COL1A2 | X02488 | Homo sapiens | collagen type I alpha 2 chain(COL1A2) |
| COL1A2 | BC054498 | Homo sapiens | collagen type I alpha 2 chain(COL1A2) |
| KIF4A | AB208797 | Homo sapiens | kinesin family member 4A(KIF4A) |
| KIF4A | BC038459 | Homo sapiens | kinesin family member 4A(KIF4A) |
| KIF4A | AK022592 | Homo sapiens | kinesin family member 4A(KIF4A) |
| KIF4A | AW592235 | Homo sapiens | kinesin family member 4A(KIF4A) |
| KIF4A | BC049218 | Homo sapiens | kinesin family member 4A(KIF4A) |
| KIF4A | AK024266 | Homo sapiens | kinesin family member 4A(KIF4A) |
| KIF4A | BC011801 | Homo sapiens | kinesin family member 4A(KIF4A) |
| KIF4A | AF071592 | Homo sapiens | kinesin family member 4A(KIF4A) |
| KIF4A | AF179308 | Homo sapiens | kinesin family member 4A(KIF4A) |
| KIF4A | AK000638 | Homo sapiens | kinesin family member 4A(KIF4A) |
| KIF4A | BC003664 | Homo sapiens | kinesin family member 4A(KIF4A) |
| KIF4A | BC050548 | Homo sapiens | kinesin family member 4A(KIF4A) |
| KIF4A | AF277375 | Homo sapiens | kinesin family member 4A(KIF4A) |
| KIF4A | AK022717 | Homo sapiens | kinesin family member 4A(KIF4A) |
| KIF4A | AK313133 | Homo sapiens | kinesin family member 4A(KIF4A) |
| KIF4A | NM_012310 | Homo sapiens | kinesin family member 4A(KIF4A) |
| KIF4A | AJ271784 | Homo sapiens | kinesin family member 4A(KIF4A) |
| KIF4A | DA021880 | Homo sapiens | kinesin family member 4A(KIF4A) |
| COL5A2 | M10956 | Homo sapiens | collagen type V alpha 2 chain(COL5A2) |
| COL5A2 | J04478 | Homo sapiens | collagen type V alpha 2 chain(COL5A2) |
| COL5A2 | BC043613 | Homo sapiens | collagen type V alpha 2 chain(COL5A2) |
| COL5A2 | M11135 | Homo sapiens | collagen type V alpha 2 chain(COL5A2) |
| COL5A2 | BC015705 | Homo sapiens | collagen type V alpha 2 chain(COL5A2) |
| COL5A2 | AK300146 | Homo sapiens | collagen type V alpha 2 chain(COL5A2) |
| COL5A2 | AL552427 | Homo sapiens | collagen type V alpha 2 chain(COL5A2) |
| COL5A2 | M31365 | Homo sapiens | collagen type V alpha 2 chain(COL5A2) |
| COL5A2 | AB209045 | Homo sapiens | collagen type V alpha 2 chain(COL5A2) |
| COL5A2 | BC086874 | Homo sapiens | collagen type V alpha 2 chain(COL5A2) |
| COL5A2 | XM_011510573 | Homo sapiens | collagen type V alpha 2 chain(COL5A2) |
| COL5A2 | AK297936 | Homo sapiens | collagen type V alpha 2 chain(COL5A2) |
| COL5A2 | X04758 | Homo sapiens | collagen type V alpha 2 chain(COL5A2) |
| COL5A2 | NM_000393 | Homo sapiens | collagen type V alpha 2 chain(COL5A2) |
| COL5A2 | Y14690 | Homo sapiens | collagen type V alpha 2 chain(COL5A2) |
| COL5A2 | M11718 | Homo sapiens | collagen type V alpha 2 chain(COL5A2) |
| FAT4 | NM_001291285 | Homo sapiens | FAT atypical cadherin 4(FAT4) |
| FAT4 | AK291461 | Homo sapiens | FAT atypical cadherin 4(FAT4) |
| FAT4 | AL713715 | Homo sapiens | FAT atypical cadherin 4(FAT4) |
| FAT4 | KJ131484 | Homo sapiens | FAT atypical cadherin 4(FAT4) |
| FAT4 | KJ131485 | Homo sapiens | FAT atypical cadherin 4(FAT4) |
| FAT4 | DT932755 | Homo sapiens | FAT atypical cadherin 4(FAT4) |
| FAT4 | KJ131486 | Homo sapiens | FAT atypical cadherin 4(FAT4) |
| FAT4 | KJ131487 | Homo sapiens | FAT atypical cadherin 4(FAT4) |
| FAT4 | NM_001291303 | Homo sapiens | FAT atypical cadherin 4(FAT4) |
| FAT4 | KJ131488 | Homo sapiens | FAT atypical cadherin 4(FAT4) |
| FAT4 | AK091292 | Homo sapiens | FAT atypical cadherin 4(FAT4) |
| FAT4 | AK096646 | Homo sapiens | FAT atypical cadherin 4(FAT4) |
| FAT4 | AK026709 | Homo sapiens | FAT atypical cadherin 4(FAT4) |
| FAT4 | DT932756 | Homo sapiens | FAT atypical cadherin 4(FAT4) |
| FAT4 | KJ131481 | Homo sapiens | FAT atypical cadherin 4(FAT4) |
| FAT4 | XM_011532236 | Homo sapiens | FAT atypical cadherin 4(FAT4) |
| FAT4 | KJ131482 | Homo sapiens | FAT atypical cadherin 4(FAT4) |
| FAT4 | KJ131483 | Homo sapiens | FAT atypical cadherin 4(FAT4) |
| FAT4 | AY356402 | Homo sapiens | FAT atypical cadherin 4(FAT4) |
| FAT4 | NM_024582 | Homo sapiens | FAT atypical cadherin 4(FAT4) |
| FAT4 | XM_011532237 | Homo sapiens | FAT atypical cadherin 4(FAT4) |
| FAT4 | AB075518 | Homo sapiens | FAT atypical cadherin 4(FAT4) |
| CD24 | BU580553 | Homo sapiens | CD24 molecule(CD24) |
| CD24 | DA753855 | Homo sapiens | CD24 molecule(CD24) |
| CD24 | DQ890636 | Homo sapiens | CD24 molecule(CD24) |
| CD24 | NR_117089 | Homo sapiens | CD24 molecule(CD24) |
| CD24 | AU125939 | Homo sapiens | CD24 molecule(CD24) |
| CD24 | NM_013230 | Homo sapiens | CD24 molecule(CD24) |
| CD24 | BT007404 | Homo sapiens | CD24 molecule(CD24) |
| CD24 | AK026603 | Homo sapiens | CD24 molecule(CD24) |
| CD24 | DA745135 | Homo sapiens | CD24 molecule(CD24) |
| CD24 | NM_001291739 | Homo sapiens | CD24 molecule(CD24) |
| CD24 | NM_001291737 | Homo sapiens | CD24 molecule(CD24) |
| CD24 | NM_001291738 | Homo sapiens | CD24 molecule(CD24) |
| CD24 | AL535013 | Homo sapiens | CD24 molecule(CD24) |
| CD24 | AK125531 | Homo sapiens | CD24 molecule(CD24) |
| CD24 | DQ893812 | Homo sapiens | CD24 molecule(CD24) |
| CD24 | X69397 | Homo sapiens | CD24 molecule(CD24) |
| CD24 | D87667 | Homo sapiens | CD24 molecule(CD24) |
| CD24 | M58664 | Homo sapiens | CD24 molecule(CD24) |
| CD24 | BG755979 | Homo sapiens | CD24 molecule(CD24) |
| CD24 | DQ530234 | Homo sapiens | CD24 molecule(CD24) |
| CD24 | DA378105 | Homo sapiens | CD24 molecule(CD24) |
| CD24 | DQ530232 | Homo sapiens | CD24 molecule(CD24) |
| CD24 | DQ530233 | Homo sapiens | CD24 molecule(CD24) |
| CD24 | DQ530230 | Homo sapiens | CD24 molecule(CD24) |
| CD24 | DQ530231 | Homo sapiens | CD24 molecule(CD24) |
| CD24 | BC064619 | Homo sapiens | CD24 molecule(CD24) |
| CD24 | BG260536 | Homo sapiens | CD24 molecule(CD24) |
| CD24 | NR_117090 | Homo sapiens | CD24 molecule(CD24) |
| CD24 | L33930 | Homo sapiens | CD24 molecule(CD24) |
| CD24 | DA518957 | Homo sapiens | CD24 molecule(CD24) |
| CD24 | DA253943 | Homo sapiens | CD24 molecule(CD24) |
| MXD3 | AF114834 | Homo sapiens | MAX dimerization protein 3(MXD3) |
| MXD3 | AK057034 | Homo sapiens | MAX dimerization protein 3(MXD3) |
| MXD3 | AK222579 | Homo sapiens | MAX dimerization protein 3(MXD3) |
| MXD3 | AK303397 | Homo sapiens | MAX dimerization protein 3(MXD3) |
| MXD3 | DQ892734 | Homo sapiens | MAX dimerization protein 3(MXD3) |
| MXD3 | NM_001142935 | Homo sapiens | MAX dimerization protein 3(MXD3) |
| MXD3 | BC041690 | Homo sapiens | MAX dimerization protein 3(MXD3) |
| MXD3 | AL833959 | Homo sapiens | MAX dimerization protein 3(MXD3) |
| MXD3 | BM849150 | Homo sapiens | MAX dimerization protein 3(MXD3) |
| MXD3 | BU620484 | Homo sapiens | MAX dimerization protein 3(MXD3) |
| MXD3 | AK092842 | Homo sapiens | MAX dimerization protein 3(MXD3) |
| MXD3 | NM_031300 | Homo sapiens | MAX dimerization protein 3(MXD3) |
| MXD3 | BC032586 | Homo sapiens | MAX dimerization protein 3(MXD3) |
| MXD3 | DQ896213 | Homo sapiens | MAX dimerization protein 3(MXD3) |
| MXD3 | AK316468 | Homo sapiens | MAX dimerization protein 3(MXD3) |
| MXD3 | BC000745 | Homo sapiens | MAX dimerization protein 3(MXD3) |
| MXD3 | AL522759 | Homo sapiens | MAX dimerization protein 3(MXD3) |
| MXD3 | DQ895981 | Homo sapiens | MAX dimerization protein 3(MXD3) |
| RNF220 | BC062600 | Homo sapiens | ring finger protein 220(RNF220) |
| RNF220 | CN285725 | Homo sapiens | ring finger protein 220(RNF220) |
| RNF220 | DC340836 | Homo sapiens | ring finger protein 220(RNF220) |
| RNF220 | XR_946692 | Homo sapiens | ring finger protein 220(RNF220) |
| RNF220 | XM_011541700 | Homo sapiens | ring finger protein 220(RNF220) |
| RNF220 | XM_011541702 | Homo sapiens | ring finger protein 220(RNF220) |
| RNF220 | NM_001319956 | Homo sapiens | ring finger protein 220(RNF220) |
| RNF220 | NM_001319957 | Homo sapiens | ring finger protein 220(RNF220) |
| RNF220 | XM_005270993 | Homo sapiens | ring finger protein 220(RNF220) |
| RNF220 | XM_017001625 | Homo sapiens | ring finger protein 220(RNF220) |
| RNF220 | XM_005270992 | Homo sapiens | ring finger protein 220(RNF220) |
| RNF220 | XM_017001626 | Homo sapiens | ring finger protein 220(RNF220) |
| RNF220 | XM_005270996 | Homo sapiens | ring finger protein 220(RNF220) |
| RNF220 | XM_017001623 | Homo sapiens | ring finger protein 220(RNF220) |
| RNF220 | BC000279 | Homo sapiens | ring finger protein 220(RNF220) |
| RNF220 | XM_017001624 | Homo sapiens | ring finger protein 220(RNF220) |
| RNF220 | BC073835 | Homo sapiens | ring finger protein 220(RNF220) |
| RNF220 | AF151080 | Homo sapiens | ring finger protein 220(RNF220) |
| RNF220 | AK293411 | Homo sapiens | ring finger protein 220(RNF220) |
| RNF220 | BC034221 | Homo sapiens | ring finger protein 220(RNF220) |
| RNF220 | AK056424 | Homo sapiens | ring finger protein 220(RNF220) |
| RNF220 | XR_001737281 | Homo sapiens | ring finger protein 220(RNF220) |
| RNF220 | XM_011541698 | Homo sapiens | ring finger protein 220(RNF220) |
| RNF220 | XM_011541699 | Homo sapiens | ring finger protein 220(RNF220) |
| RNF220 | NM_018150 | Homo sapiens | ring finger protein 220(RNF220) |
| RNF220 | AK025744 | Homo sapiens | ring finger protein 220(RNF220) |
| RNF220 | XR_001737280 | Homo sapiens | ring finger protein 220(RNF220) |
| RNF220 | BC098300 | Homo sapiens | ring finger protein 220(RNF220) |
| RNF220 | BC105790 | Homo sapiens | ring finger protein 220(RNF220) |
| RNF220 | XR_001737278 | Homo sapiens | ring finger protein 220(RNF220) |
| RNF220 | BC098266 | Homo sapiens | ring finger protein 220(RNF220) |
| RNF220 | XR_001737279 | Homo sapiens | ring finger protein 220(RNF220) |
| RNF220 | AK001459 | Homo sapiens | ring finger protein 220(RNF220) |
| RNF220 | XM_006710735 | Homo sapiens | ring finger protein 220(RNF220) |
| RNF220 | AK124790 | Homo sapiens | ring finger protein 220(RNF220) |
| RNF220 | AK225180 | Homo sapiens | ring finger protein 220(RNF220) |
| RNF220 | AK297228 | Homo sapiens | ring finger protein 220(RNF220) |
| RNF220 | AK296038 | Homo sapiens | ring finger protein 220(RNF220) |
| SNAP25 | L19760 | Homo sapiens | synaptosome associated protein 25(SNAP25) |
| SNAP25 | XM_017028021 | Homo sapiens | synaptosome associated protein 25(SNAP25) |
| SNAP25 | XM_017028022 | Homo sapiens | synaptosome associated protein 25(SNAP25) |
| SNAP25 | L19761 | Homo sapiens | synaptosome associated protein 25(SNAP25) |
| SNAP25 | XM_017028023 | Homo sapiens | synaptosome associated protein 25(SNAP25) |
| SNAP25 | HY127744 | Homo sapiens | synaptosome associated protein 25(SNAP25) |
| SNAP25 | NM_001322909 | Homo sapiens | synaptosome associated protein 25(SNAP25) |
| SNAP25 | DA072367 | Homo sapiens | synaptosome associated protein 25(SNAP25) |
| SNAP25 | XM_005260808 | Homo sapiens | synaptosome associated protein 25(SNAP25) |
| SNAP25 | BC010647 | Homo sapiens | synaptosome associated protein 25(SNAP25) |
| SNAP25 | NM_130811 | Homo sapiens | synaptosome associated protein 25(SNAP25) |
| SNAP25 | AK314359 | Homo sapiens | synaptosome associated protein 25(SNAP25) |
| SNAP25 | AK090857 | Homo sapiens | synaptosome associated protein 25(SNAP25) |
| SNAP25 | NM_001322910 | Homo sapiens | synaptosome associated protein 25(SNAP25) |
| SNAP25 | AM393230 | Homo sapiens | synaptosome associated protein 25(SNAP25) |
| SNAP25 | BT019684 | Homo sapiens | synaptosome associated protein 25(SNAP25) |
| SNAP25 | HQ447952 | Homo sapiens | synaptosome associated protein 25(SNAP25) |
| SNAP25 | BM666216 | Homo sapiens | synaptosome associated protein 25(SNAP25) |
| SNAP25 | AK094560 | Homo sapiens | synaptosome associated protein 25(SNAP25) |
| SNAP25 | BI604120 | Homo sapiens | synaptosome associated protein 25(SNAP25) |
| SNAP25 | BI552777 | Homo sapiens | synaptosome associated protein 25(SNAP25) |
| SNAP25 | BI602062 | Homo sapiens | synaptosome associated protein 25(SNAP25) |
| SNAP25 | AK026732 | Homo sapiens | synaptosome associated protein 25(SNAP25) |
| SNAP25 | NM_001322902 | Homo sapiens | synaptosome associated protein 25(SNAP25) |
| SNAP25 | AK289647 | Homo sapiens | synaptosome associated protein 25(SNAP25) |
| SNAP25 | D21267 | Homo sapiens | synaptosome associated protein 25(SNAP25) |
| SNAP25 | DA162693 | Homo sapiens | synaptosome associated protein 25(SNAP25) |
| SNAP25 | NM_001322904 | Homo sapiens | synaptosome associated protein 25(SNAP25) |
| SNAP25 | NM_001322903 | Homo sapiens | synaptosome associated protein 25(SNAP25) |
| SNAP25 | AK098347 | Homo sapiens | synaptosome associated protein 25(SNAP25) |
| SNAP25 | NM_001322906 | Homo sapiens | synaptosome associated protein 25(SNAP25) |
| SNAP25 | NM_001322905 | Homo sapiens | synaptosome associated protein 25(SNAP25) |
| SNAP25 | AK098767 | Homo sapiens | synaptosome associated protein 25(SNAP25) |
| SNAP25 | NM_001322908 | Homo sapiens | synaptosome associated protein 25(SNAP25) |
| SNAP25 | NM_001322907 | Homo sapiens | synaptosome associated protein 25(SNAP25) |
| SNAP25 | AK223617 | Homo sapiens | synaptosome associated protein 25(SNAP25) |
| SNAP25 | AM393653 | Homo sapiens | synaptosome associated protein 25(SNAP25) |
| SNAP25 | BX647443 | Homo sapiens | synaptosome associated protein 25(SNAP25) |
| SNAP25 | NM_003081 | Homo sapiens | synaptosome associated protein 25(SNAP25) |
| FOXM1 | NM_001243088 | Homo sapiens | forkhead box M1(FOXM1) |
| FOXM1 | NM_001243089 | Homo sapiens | forkhead box M1(FOXM1) |
| FOXM1 | AY542306 | Homo sapiens | forkhead box M1(FOXM1) |
| FOXM1 | BC012863 | Homo sapiens | forkhead box M1(FOXM1) |
| FOXM1 | NM_021953 | Homo sapiens | forkhead box M1(FOXM1) |
| FOXM1 | U74613 | Homo sapiens | forkhead box M1(FOXM1) |
| FOXM1 | U74612 | Homo sapiens | forkhead box M1(FOXM1) |
| FOXM1 | AW780338 | Homo sapiens | forkhead box M1(FOXM1) |
| FOXM1 | BC035437 | Homo sapiens | forkhead box M1(FOXM1) |
| FOXM1 | XM_005253676 | Homo sapiens | forkhead box M1(FOXM1) |
| FOXM1 | BQ229497 | Homo sapiens | forkhead box M1(FOXM1) |
| FOXM1 | L16783 | Homo sapiens | forkhead box M1(FOXM1) |
| FOXM1 | AK313845 | Homo sapiens | forkhead box M1(FOXM1) |
| FOXM1 | BC006192 | Homo sapiens | forkhead box M1(FOXM1) |
| FOXM1 | XM_011520932 | Homo sapiens | forkhead box M1(FOXM1) |
| FOXM1 | XM_011520931 | Homo sapiens | forkhead box M1(FOXM1) |
| FOXM1 | XM_011520930 | Homo sapiens | forkhead box M1(FOXM1) |
| FOXM1 | XR_931507 | Homo sapiens | forkhead box M1(FOXM1) |
| FOXM1 | BT006986 | Homo sapiens | forkhead box M1(FOXM1) |
| FOXM1 | NM_202002 | Homo sapiens | forkhead box M1(FOXM1) |
| FOXM1 | NM_202003 | Homo sapiens | forkhead box M1(FOXM1) |
| FOXM1 | XM_011520935 | Homo sapiens | forkhead box M1(FOXM1) |
| FOXM1 | XM_011520934 | Homo sapiens | forkhead box M1(FOXM1) |
| FOXM1 | XM_011520933 | Homo sapiens | forkhead box M1(FOXM1) |
| FOXM1 | BC006529 | Homo sapiens | forkhead box M1(FOXM1) |
| FOXM1 | DB137846 | Homo sapiens | forkhead box M1(FOXM1) |
| FOXM1 | AK291206 | Homo sapiens | forkhead box M1(FOXM1) |
| FOXM1 | JF432495 | Homo sapiens | forkhead box M1(FOXM1) |
| FOXM1 | U83113 | Homo sapiens | forkhead box M1(FOXM1) |
| CHD1 | AF006513 | Homo sapiens | chromodomain helicase DNA binding protein 1(CHD1) |
| CHD1 | BC117134 | Homo sapiens | chromodomain helicase DNA binding protein 1(CHD1) |
| CHD1 | XM_011543112 | Homo sapiens | chromodomain helicase DNA binding protein 1(CHD1) |
| CHD1 | CN277742 | Homo sapiens | chromodomain helicase DNA binding protein 1(CHD1) |
| CHD1 | XR_427702 | Homo sapiens | chromodomain helicase DNA binding protein 1(CHD1) |
| CHD1 | XM_017008991 | Homo sapiens | chromodomain helicase DNA binding protein 1(CHD1) |
| CHD1 | BQ436172 | Homo sapiens | chromodomain helicase DNA binding protein 1(CHD1) |
| CHD1 | XM_017008992 | Homo sapiens | chromodomain helicase DNA binding protein 1(CHD1) |
| CHD1 | XR_001741986 | Homo sapiens | chromodomain helicase DNA binding protein 1(CHD1) |
| CHD1 | NM_001270 | Homo sapiens | chromodomain helicase DNA binding protein 1(CHD1) |
| CHD1 | XM_005271866 | Homo sapiens | chromodomain helicase DNA binding protein 1(CHD1) |
| CHD1 | AK094871 | Homo sapiens | chromodomain helicase DNA binding protein 1(CHD1) |
| CHD1 | BX648767 | Homo sapiens | chromodomain helicase DNA binding protein 1(CHD1) |
| CHD1 | XM_005271867 | Homo sapiens | chromodomain helicase DNA binding protein 1(CHD1) |
| CHD1 | BM698633 | Homo sapiens | chromodomain helicase DNA binding protein 1(CHD1) |
| CHD1 | BC054860 | Homo sapiens | chromodomain helicase DNA binding protein 1(CHD1) |
| ELAVL2 | XM_017014418 | Homo sapiens | ELAV like RNA binding protein 2(ELAVL2) |
| ELAVL2 | XM_017014419 | Homo sapiens | ELAV like RNA binding protein 2(ELAVL2) |
| ELAVL2 | XR_929211 | Homo sapiens | ELAV like RNA binding protein 2(ELAVL2) |
| ELAVL2 | XM_017014414 | Homo sapiens | ELAV like RNA binding protein 2(ELAVL2) |
| ELAVL2 | XM_017014415 | Homo sapiens | ELAV like RNA binding protein 2(ELAVL2) |
| ELAVL2 | XM_017014416 | Homo sapiens | ELAV like RNA binding protein 2(ELAVL2) |
| ELAVL2 | XM_011517774 | Homo sapiens | ELAV like RNA binding protein 2(ELAVL2) |
| ELAVL2 | XM_017014417 | Homo sapiens | ELAV like RNA binding protein 2(ELAVL2) |
| ELAVL2 | AB209294 | Homo sapiens | ELAV like RNA binding protein 2(ELAVL2) |
| ELAVL2 | BC042393 | Homo sapiens | ELAV like RNA binding protein 2(ELAVL2) |
| ELAVL2 | XM_005251395 | Homo sapiens | ELAV like RNA binding protein 2(ELAVL2) |
| ELAVL2 | XM_005251394 | Homo sapiens | ELAV like RNA binding protein 2(ELAVL2) |
| ELAVL2 | XM_005251393 | Homo sapiens | ELAV like RNA binding protein 2(ELAVL2) |
| ELAVL2 | XR_929212 | Homo sapiens | ELAV like RNA binding protein 2(ELAVL2) |
| ELAVL2 | NM_001171197 | Homo sapiens | ELAV like RNA binding protein 2(ELAVL2) |
| ELAVL2 | NM_001171195 | Homo sapiens | ELAV like RNA binding protein 2(ELAVL2) |
| ELAVL2 | XM_011517786 | Homo sapiens | ELAV like RNA binding protein 2(ELAVL2) |
| ELAVL2 | XM_017014410 | Homo sapiens | ELAV like RNA binding protein 2(ELAVL2) |
| ELAVL2 | XM_017014411 | Homo sapiens | ELAV like RNA binding protein 2(ELAVL2) |
| ELAVL2 | XM_017014412 | Homo sapiens | ELAV like RNA binding protein 2(ELAVL2) |
| ELAVL2 | XM_017014413 | Homo sapiens | ELAV like RNA binding protein 2(ELAVL2) |
| ELAVL2 | XM_017014408 | Homo sapiens | ELAV like RNA binding protein 2(ELAVL2) |
| ELAVL2 | XM_011517780 | Homo sapiens | ELAV like RNA binding protein 2(ELAVL2) |
| ELAVL2 | XM_017014409 | Homo sapiens | ELAV like RNA binding protein 2(ELAVL2) |
| ELAVL2 | XM_017014425 | Homo sapiens | ELAV like RNA binding protein 2(ELAVL2) |
| ELAVL2 | XM_011517783 | Homo sapiens | ELAV like RNA binding protein 2(ELAVL2) |
| ELAVL2 | XM_017014426 | Homo sapiens | ELAV like RNA binding protein 2(ELAVL2) |
| ELAVL2 | XM_011517784 | Homo sapiens | ELAV like RNA binding protein 2(ELAVL2) |
| ELAVL2 | XM_011517785 | Homo sapiens | ELAV like RNA binding protein 2(ELAVL2) |
| ELAVL2 | DQ894809 | Homo sapiens | ELAV like RNA binding protein 2(ELAVL2) |
| ELAVL2 | DQ891617 | Homo sapiens | ELAV like RNA binding protein 2(ELAVL2) |
| ELAVL2 | U13706 | Homo sapiens | ELAV like RNA binding protein 2(ELAVL2) |
| ELAVL2 | XM_006716736 | Homo sapiens | ELAV like RNA binding protein 2(ELAVL2) |
| ELAVL2 | NM_004432 | Homo sapiens | ELAV like RNA binding protein 2(ELAVL2) |
| ELAVL2 | XM_006716735 | Homo sapiens | ELAV like RNA binding protein 2(ELAVL2) |
| ELAVL2 | XM_006716734 | Homo sapiens | ELAV like RNA binding protein 2(ELAVL2) |
| ELAVL2 | XM_017014421 | Homo sapiens | ELAV like RNA binding protein 2(ELAVL2) |
| ELAVL2 | EU176647 | Homo sapiens | ELAV like RNA binding protein 2(ELAVL2) |
| ELAVL2 | XM_011517776 | Homo sapiens | ELAV like RNA binding protein 2(ELAVL2) |
| ELAVL2 | XM_017014422 | Homo sapiens | ELAV like RNA binding protein 2(ELAVL2) |
| ELAVL2 | XM_011517777 | Homo sapiens | ELAV like RNA binding protein 2(ELAVL2) |
| ELAVL2 | XM_017014423 | Homo sapiens | ELAV like RNA binding protein 2(ELAVL2) |
| ELAVL2 | XM_011517778 | Homo sapiens | ELAV like RNA binding protein 2(ELAVL2) |
| ELAVL2 | XM_017014424 | Homo sapiens | ELAV like RNA binding protein 2(ELAVL2) |
| ELAVL2 | BC035004 | Homo sapiens | ELAV like RNA binding protein 2(ELAVL2) |
| ELAVL2 | XM_011517779 | Homo sapiens | ELAV like RNA binding protein 2(ELAVL2) |
| ELAVL2 | BC030692 | Homo sapiens | ELAV like RNA binding protein 2(ELAVL2) |
| ELAVL2 | XM_017014420 | Homo sapiens | ELAV like RNA binding protein 2(ELAVL2) |
| SHH | XM_011516480 | Homo sapiens | sonic hedgehog(SHH) |
| SHH | BX452210 | Homo sapiens | sonic hedgehog(SHH) |
| SHH | NR_132318 | Homo sapiens | sonic hedgehog(SHH) |
| SHH | NR_132319 | Homo sapiens | sonic hedgehog(SHH) |
| SHH | AI192528 | Homo sapiens | sonic hedgehog(SHH) |
| SHH | L38518 | Homo sapiens | sonic hedgehog(SHH) |
| SHH | NM_001310462 | Homo sapiens | sonic hedgehog(SHH) |
| SHH | AY927454 | Homo sapiens | sonic hedgehog(SHH) |
| SHH | AY927453 | Homo sapiens | sonic hedgehog(SHH) |
| SHH | BC111925 | Homo sapiens | sonic hedgehog(SHH) |
| SHH | XM_011516479 | Homo sapiens | sonic hedgehog(SHH) |
| SHH | AY927455 | Homo sapiens | sonic hedgehog(SHH) |
| SHH | AY927450 | Homo sapiens | sonic hedgehog(SHH) |
| SHH | BX109787 | Homo sapiens | sonic hedgehog(SHH) |
| SHH | NM_000193 | Homo sapiens | sonic hedgehog(SHH) |
| SHH | BY797055 | Homo sapiens | sonic hedgehog(SHH) |
| SHH | AY927452 | Homo sapiens | sonic hedgehog(SHH) |
| SHH | AY927451 | Homo sapiens | sonic hedgehog(SHH) |
| EPB41L4A | XM_017009689 | Homo sapiens | erythrocyte membrane protein band 4.1 like 4A(EPB41L4A) |
| EPB41L4A | AL117425 | Homo sapiens | erythrocyte membrane protein band 4.1 like 4A(EPB41L4A) |
| EPB41L4A | XM_011543530 | Homo sapiens | erythrocyte membrane protein band 4.1 like 4A(EPB41L4A) |
| EPB41L4A | XR_001742173 | Homo sapiens | erythrocyte membrane protein band 4.1 like 4A(EPB41L4A) |
| EPB41L4A | AA406206 | Homo sapiens | erythrocyte membrane protein band 4.1 like 4A(EPB41L4A) |
| EPB41L4A | XM_011543531 | Homo sapiens | erythrocyte membrane protein band 4.1 like 4A(EPB41L4A) |
| EPB41L4A | XM_011543532 | Homo sapiens | erythrocyte membrane protein band 4.1 like 4A(EPB41L4A) |
| EPB41L4A | XR_001742175 | Homo sapiens | erythrocyte membrane protein band 4.1 like 4A(EPB41L4A) |
| EPB41L4A | XM_011543533 | Homo sapiens | erythrocyte membrane protein band 4.1 like 4A(EPB41L4A) |
| EPB41L4A | AK096057 | Homo sapiens | erythrocyte membrane protein band 4.1 like 4A(EPB41L4A) |
| EPB41L4A | CB048933 | Homo sapiens | erythrocyte membrane protein band 4.1 like 4A(EPB41L4A) |
| EPB41L4A | AA350575 | Homo sapiens | erythrocyte membrane protein band 4.1 like 4A(EPB41L4A) |
| EPB41L4A | AK021578 | Homo sapiens | erythrocyte membrane protein band 4.1 like 4A(EPB41L4A) |
| EPB41L4A | JF432740 | Homo sapiens | erythrocyte membrane protein band 4.1 like 4A(EPB41L4A) |
| EPB41L4A | AK123285 | Homo sapiens | erythrocyte membrane protein band 4.1 like 4A(EPB41L4A) |
| EPB41L4A | BC114942 | Homo sapiens | erythrocyte membrane protein band 4.1 like 4A(EPB41L4A) |
| EPB41L4A | BC114632 | Homo sapiens | erythrocyte membrane protein band 4.1 like 4A(EPB41L4A) |
| EPB41L4A | AB030240 | Homo sapiens | erythrocyte membrane protein band 4.1 like 4A(EPB41L4A) |
| EPB41L4A | NM_022140 | Homo sapiens | erythrocyte membrane protein band 4.1 like 4A(EPB41L4A) |
| EPB41L4A | BC160044 | Homo sapiens | erythrocyte membrane protein band 4.1 like 4A(EPB41L4A) |
| EPB41L4A | BC031042 | Homo sapiens | erythrocyte membrane protein band 4.1 like 4A(EPB41L4A) |
| EPB41L4A | BC015441 | Homo sapiens | erythrocyte membrane protein band 4.1 like 4A(EPB41L4A) |
| ADAMTS3 | AF247668 | Homo sapiens | ADAM metallopeptidase with thrombospondin type 1 motif 3(ADAMTS3) |
| ADAMTS3 | BC019707 | Homo sapiens | ADAM metallopeptidase with thrombospondin type 1 motif 3(ADAMTS3) |
| ADAMTS3 | XM_011532422 | Homo sapiens | ADAM metallopeptidase with thrombospondin type 1 motif 3(ADAMTS3) |
| ADAMTS3 | AK295131 | Homo sapiens | ADAM metallopeptidase with thrombospondin type 1 motif 3(ADAMTS3) |
| ADAMTS3 | BC130287 | Homo sapiens | ADAM metallopeptidase with thrombospondin type 1 motif 3(ADAMTS3) |
| ADAMTS3 | XM_011532421 | Homo sapiens | ADAM metallopeptidase with thrombospondin type 1 motif 3(ADAMTS3) |
| ADAMTS3 | BC132735 | Homo sapiens | ADAM metallopeptidase with thrombospondin type 1 motif 3(ADAMTS3) |
| ADAMTS3 | NM_014243 | Homo sapiens | ADAM metallopeptidase with thrombospondin type 1 motif 3(ADAMTS3) |
| ADAMTS3 | AB002364 | Homo sapiens | ADAM metallopeptidase with thrombospondin type 1 motif 3(ADAMTS3) |
| ADAMTS3 | BC016451 | Homo sapiens | ADAM metallopeptidase with thrombospondin type 1 motif 3(ADAMTS3) |
| SIX3 | AJ012611 | Homo sapiens | SIX homeobox 3(SIX3) |
| SIX3 | BF569811 | Homo sapiens | SIX homeobox 3(SIX3) |
| SIX3 | EL952320 | Homo sapiens | SIX homeobox 3(SIX3) |
| SIX3 | BF570321 | Homo sapiens | SIX homeobox 3(SIX3) |
| SIX3 | CD673488 | Homo sapiens | SIX homeobox 3(SIX3) |
| SIX3 | BC153026 | Homo sapiens | SIX homeobox 3(SIX3) |
| SIX3 | HG493938 | Homo sapiens | SIX homeobox 3(SIX3) |
| SIX3 | BC030289 | Homo sapiens | SIX homeobox 3(SIX3) |
| SIX3 | NM_005413 | Homo sapiens | SIX homeobox 3(SIX3) |
| SIX3 | AL162671 | Homo sapiens | SIX homeobox 3(SIX3) |
| SIX3 | BM668514 | Homo sapiens | SIX homeobox 3(SIX3) |
| GRM8 | BC093725 | Homo sapiens | glutamate metabotropic receptor 8(GRM8) |
| GRM8 | XM_011516102 | Homo sapiens | glutamate metabotropic receptor 8(GRM8) |
| GRM8 | XM_011516101 | Homo sapiens | glutamate metabotropic receptor 8(GRM8) |
| GRM8 | BC101675 | Homo sapiens | glutamate metabotropic receptor 8(GRM8) |
| GRM8 | XM_006715938 | Homo sapiens | glutamate metabotropic receptor 8(GRM8) |
| GRM8 | AK123053 | Homo sapiens | glutamate metabotropic receptor 8(GRM8) |
| GRM8 | AY608335 | Homo sapiens | glutamate metabotropic receptor 8(GRM8) |
| GRM8 | XM_017012077 | Homo sapiens | glutamate metabotropic receptor 8(GRM8) |
| GRM8 | XM_017012076 | Homo sapiens | glutamate metabotropic receptor 8(GRM8) |
| GRM8 | AK315203 | Homo sapiens | glutamate metabotropic receptor 8(GRM8) |
| GRM8 | XM_017012075 | Homo sapiens | glutamate metabotropic receptor 8(GRM8) |
| GRM8 | XM_017012074 | Homo sapiens | glutamate metabotropic receptor 8(GRM8) |
| GRM8 | AJ236922 | Homo sapiens | glutamate metabotropic receptor 8(GRM8) |
| GRM8 | XM_017012079 | Homo sapiens | glutamate metabotropic receptor 8(GRM8) |
| GRM8 | AJ236921 | Homo sapiens | glutamate metabotropic receptor 8(GRM8) |
| GRM8 | XM_017012078 | Homo sapiens | glutamate metabotropic receptor 8(GRM8) |
| GRM8 | XM_011516094 | Homo sapiens | glutamate metabotropic receptor 8(GRM8) |
| GRM8 | XM_011516092 | Homo sapiens | glutamate metabotropic receptor 8(GRM8) |
| GRM8 | XM_011516091 | Homo sapiens | glutamate metabotropic receptor 8(GRM8) |
| GRM8 | BC143344 | Homo sapiens | glutamate metabotropic receptor 8(GRM8) |
| GRM8 | U95025 | Homo sapiens | glutamate metabotropic receptor 8(GRM8) |
| GRM8 | BG211606 | Homo sapiens | glutamate metabotropic receptor 8(GRM8) |
| GRM8 | U92459 | Homo sapiens | glutamate metabotropic receptor 8(GRM8) |
| GRM8 | NM_000845 | Homo sapiens | glutamate metabotropic receptor 8(GRM8) |
| GRM8 | NM_001127323 | Homo sapiens | glutamate metabotropic receptor 8(GRM8) |
| GRM8 | XM_011516095 | Homo sapiens | glutamate metabotropic receptor 8(GRM8) |
| GRM8 | BG202742 | Homo sapiens | glutamate metabotropic receptor 8(GRM8) |
| GRM8 | XM_017012080 | Homo sapiens | glutamate metabotropic receptor 8(GRM8) |
| GRM8 | XM_011516108 | Homo sapiens | glutamate metabotropic receptor 8(GRM8) |
| GRM8 | EU432125 | Homo sapiens | glutamate metabotropic receptor 8(GRM8) |
| GRM8 | XM_011516103 | Homo sapiens | glutamate metabotropic receptor 8(GRM8) |
| GRM8 | AK290197 | Homo sapiens | glutamate metabotropic receptor 8(GRM8) |
| GRM8 | NR_028041 | Homo sapiens | glutamate metabotropic receptor 8(GRM8) |
| CFAP47 | BG197183 | Homo sapiens | cilia and flagella associated protein 47(CFAP47) |
| CFAP47 | CD686506 | Homo sapiens | cilia and flagella associated protein 47(CFAP47) |
| CFAP47 | DV080631 | Homo sapiens | cilia and flagella associated protein 47(CFAP47) |
| CFAP47 | XM_017029452 | Homo sapiens | cilia and flagella associated protein 47(CFAP47) |
| CFAP47 | DV080632 | Homo sapiens | cilia and flagella associated protein 47(CFAP47) |
| CFAP47 | XM_017029453 | Homo sapiens | cilia and flagella associated protein 47(CFAP47) |
| CFAP47 | AI648401 | Homo sapiens | cilia and flagella associated protein 47(CFAP47) |
| CFAP47 | BC101698 | Homo sapiens | cilia and flagella associated protein 47(CFAP47) |
| CFAP47 | DY655297 | Homo sapiens | cilia and flagella associated protein 47(CFAP47) |
| CFAP47 | BG190566 | Homo sapiens | cilia and flagella associated protein 47(CFAP47) |
| CFAP47 | BG200277 | Homo sapiens | cilia and flagella associated protein 47(CFAP47) |
| CFAP47 | CD106515 | Homo sapiens | cilia and flagella associated protein 47(CFAP47) |
| CFAP47 | AK093920 | Homo sapiens | cilia and flagella associated protein 47(CFAP47) |
| CFAP47 | DA315191 | Homo sapiens | cilia and flagella associated protein 47(CFAP47) |
| CFAP47 | BC027936 | Homo sapiens | cilia and flagella associated protein 47(CFAP47) |
| CFAP47 | DV080105 | Homo sapiens | cilia and flagella associated protein 47(CFAP47) |
| CFAP47 | DV080305 | Homo sapiens | cilia and flagella associated protein 47(CFAP47) |
| CFAP47 | NM_001304548 | Homo sapiens | cilia and flagella associated protein 47(CFAP47) |
| CFAP47 | DV080684 | Homo sapiens | cilia and flagella associated protein 47(CFAP47) |
| CFAP47 | NM_152632 | Homo sapiens | cilia and flagella associated protein 47(CFAP47) |
| CFAP47 | DV080141 | Homo sapiens | cilia and flagella associated protein 47(CFAP47) |
| CFAP47 | DV080142 | Homo sapiens | cilia and flagella associated protein 47(CFAP47) |
| CFAP47 | NM_173695 | Homo sapiens | cilia and flagella associated protein 47(CFAP47) |
| CFAP47 | AI822140 | Homo sapiens | cilia and flagella associated protein 47(CFAP47) |
| CFAP47 | DV080683 | Homo sapiens | cilia and flagella associated protein 47(CFAP47) |
| CFAP47 | BX111076 | Homo sapiens | cilia and flagella associated protein 47(CFAP47) |
| CFAP47 | BC101700 | Homo sapiens | cilia and flagella associated protein 47(CFAP47) |
| CFAP47 | AK126295 | Homo sapiens | cilia and flagella associated protein 47(CFAP47) |
| CFAP47 | DV080519 | Homo sapiens | cilia and flagella associated protein 47(CFAP47) |
| CFAP47 | DV080613 | Homo sapiens | cilia and flagella associated protein 47(CFAP47) |
| CFAP47 | BG186336 | Homo sapiens | cilia and flagella associated protein 47(CFAP47) |
| HYDIN | AK057467 | Homo sapiens | HYDIN, axonemal central pair apparatus protein(HYDIN) |
| HYDIN | DA758908 | Homo sapiens | HYDIN, axonemal central pair apparatus protein(HYDIN) |
| HYDIN | AK299016 | Homo sapiens | HYDIN, axonemal central pair apparatus protein(HYDIN) |
| HYDIN | XM_006721206 | Homo sapiens | HYDIN, axonemal central pair apparatus protein(HYDIN) |
| HYDIN | XM_011523155 | Homo sapiens | HYDIN, axonemal central pair apparatus protein(HYDIN) |
| HYDIN | AL137259 | Homo sapiens | HYDIN, axonemal central pair apparatus protein(HYDIN) |
| HYDIN | BC028351 | Homo sapiens | HYDIN, axonemal central pair apparatus protein(HYDIN) |
| HYDIN | AK026688 | Homo sapiens | HYDIN, axonemal central pair apparatus protein(HYDIN) |
| HYDIN | JX501991 | Homo sapiens | HYDIN, axonemal central pair apparatus protein(HYDIN) |
| HYDIN | DB339472 | Homo sapiens | HYDIN, axonemal central pair apparatus protein(HYDIN) |
| HYDIN | AL122038 | Homo sapiens | HYDIN, axonemal central pair apparatus protein(HYDIN) |
| HYDIN | DB079205 | Homo sapiens | HYDIN, axonemal central pair apparatus protein(HYDIN) |
| HYDIN | NM_032821 | Homo sapiens | HYDIN, axonemal central pair apparatus protein(HYDIN) |
| HYDIN | AL705531 | Homo sapiens | HYDIN, axonemal central pair apparatus protein(HYDIN) |
| HYDIN | AK299348 | Homo sapiens | HYDIN, axonemal central pair apparatus protein(HYDIN) |
| HYDIN | XM_011523147 | Homo sapiens | HYDIN, axonemal central pair apparatus protein(HYDIN) |
| HYDIN | XM_011523148 | Homo sapiens | HYDIN, axonemal central pair apparatus protein(HYDIN) |
| HYDIN | AL133042 | Homo sapiens | HYDIN, axonemal central pair apparatus protein(HYDIN) |
| HYDIN | NM_001270974 | Homo sapiens | HYDIN, axonemal central pair apparatus protein(HYDIN) |
| HYDIN | XM_011523146 | Homo sapiens | HYDIN, axonemal central pair apparatus protein(HYDIN) |
| HYDIN | BP228881 | Homo sapiens | HYDIN, axonemal central pair apparatus protein(HYDIN) |
| HYDIN | AK074472 | Homo sapiens | HYDIN, axonemal central pair apparatus protein(HYDIN) |
| HYDIN | BC043273 | Homo sapiens | HYDIN, axonemal central pair apparatus protein(HYDIN) |
| HYDIN | HG508232 | Homo sapiens | HYDIN, axonemal central pair apparatus protein(HYDIN) |
| HYDIN | NM_001198542 | Homo sapiens | HYDIN, axonemal central pair apparatus protein(HYDIN) |
| HYDIN | XM_017023347 | Homo sapiens | HYDIN, axonemal central pair apparatus protein(HYDIN) |
| HYDIN | NM_001198543 | Homo sapiens | HYDIN, axonemal central pair apparatus protein(HYDIN) |
| HYDIN | XM_017023346 | Homo sapiens | HYDIN, axonemal central pair apparatus protein(HYDIN) |
| HYDIN | AK022933 | Homo sapiens | HYDIN, axonemal central pair apparatus protein(HYDIN) |
| HYDIN | XM_017023348 | Homo sapiens | HYDIN, axonemal central pair apparatus protein(HYDIN) |
| HYDIN | XM_011523151 | Homo sapiens | HYDIN, axonemal central pair apparatus protein(HYDIN) |
| HYDIN | AK125886 | Homo sapiens | HYDIN, axonemal central pair apparatus protein(HYDIN) |
| HYDIN | XM_011523152 | Homo sapiens | HYDIN, axonemal central pair apparatus protein(HYDIN) |
| HYDIN | AK308296 | Homo sapiens | HYDIN, axonemal central pair apparatus protein(HYDIN) |
| HYDIN | NM_017558 | Homo sapiens | HYDIN, axonemal central pair apparatus protein(HYDIN) |
| RSPO3 | XM_017011378 | Homo sapiens | R-spondin 3(RSPO3) |
| RSPO3 | XM_017011379 | Homo sapiens | R-spondin 3(RSPO3) |
| RSPO3 | NM_032784 | Homo sapiens | R-spondin 3(RSPO3) |
| RSPO3 | DQ894583 | Homo sapiens | R-spondin 3(RSPO3) |
| RSPO3 | AA568446 | Homo sapiens | R-spondin 3(RSPO3) |
| RSPO3 | AI095468 | Homo sapiens | R-spondin 3(RSPO3) |
| RSPO3 | BC022367 | Homo sapiens | R-spondin 3(RSPO3) |
| RSPO3 | DQ891407 | Homo sapiens | R-spondin 3(RSPO3) |
| RSPO3 | AF251057 | Homo sapiens | R-spondin 3(RSPO3) |
| RSPO3 | AF086298 | Homo sapiens | R-spondin 3(RSPO3) |
| RSPO3 | AK314912 | Homo sapiens | R-spondin 3(RSPO3) |
| KDM6A | AF000992 | Homo sapiens | lysine demethylase 6A(KDM6A) |
| KDM6A | BC093868 | Homo sapiens | lysine demethylase 6A(KDM6A) |
| KDM6A | AK304114 | Homo sapiens | lysine demethylase 6A(KDM6A) |
| KDM6A | AF000993 | Homo sapiens | lysine demethylase 6A(KDM6A) |
| KDM6A | EU026283 | Homo sapiens | lysine demethylase 6A(KDM6A) |
| KDM6A | NM_001291421 | Homo sapiens | lysine demethylase 6A(KDM6A) |
| KDM6A | NR_111960 | Homo sapiens | lysine demethylase 6A(KDM6A) |
| KDM6A | AL041377 | Homo sapiens | lysine demethylase 6A(KDM6A) |
| KDM6A | CR749602 | Homo sapiens | lysine demethylase 6A(KDM6A) |
| KDM6A | NM_001291418 | Homo sapiens | lysine demethylase 6A(KDM6A) |
| KDM6A | NM_001291416 | Homo sapiens | lysine demethylase 6A(KDM6A) |
| KDM6A | XM_005272656 | Homo sapiens | lysine demethylase 6A(KDM6A) |
| KDM6A | NM_001291417 | Homo sapiens | lysine demethylase 6A(KDM6A) |
| KDM6A | XM_005272659 | Homo sapiens | lysine demethylase 6A(KDM6A) |
| KDM6A | XR_001755723 | Homo sapiens | lysine demethylase 6A(KDM6A) |
| KDM6A | XM_011543957 | Homo sapiens | lysine demethylase 6A(KDM6A) |
| KDM6A | XM_011543959 | Homo sapiens | lysine demethylase 6A(KDM6A) |
| KDM6A | XM_011543958 | Homo sapiens | lysine demethylase 6A(KDM6A) |
| KDM6A | AK294456 | Homo sapiens | lysine demethylase 6A(KDM6A) |
| KDM6A | BC143275 | Homo sapiens | lysine demethylase 6A(KDM6A) |
| KDM6A | BC143277 | Homo sapiens | lysine demethylase 6A(KDM6A) |
| KDM6A | XM_011543960 | Homo sapiens | lysine demethylase 6A(KDM6A) |
| KDM6A | BC143278 | Homo sapiens | lysine demethylase 6A(KDM6A) |
| KDM6A | XM_011543962 | Homo sapiens | lysine demethylase 6A(KDM6A) |
| KDM6A | XM_011543961 | Homo sapiens | lysine demethylase 6A(KDM6A) |
| KDM6A | XM_011543964 | Homo sapiens | lysine demethylase 6A(KDM6A) |
| KDM6A | XM_011543963 | Homo sapiens | lysine demethylase 6A(KDM6A) |
| KDM6A | XM_011543966 | Homo sapiens | lysine demethylase 6A(KDM6A) |
| KDM6A | XM_011543965 | Homo sapiens | lysine demethylase 6A(KDM6A) |
| KDM6A | BC113381 | Homo sapiens | lysine demethylase 6A(KDM6A) |
| KDM6A | XM_011543968 | Homo sapiens | lysine demethylase 6A(KDM6A) |
| KDM6A | XM_011543967 | Homo sapiens | lysine demethylase 6A(KDM6A) |
| KDM6A | XM_011543969 | Homo sapiens | lysine demethylase 6A(KDM6A) |
| KDM6A | AL831996 | Homo sapiens | lysine demethylase 6A(KDM6A) |
| KDM6A | DQ062677 | Homo sapiens | lysine demethylase 6A(KDM6A) |
| KDM6A | DQ062676 | Homo sapiens | lysine demethylase 6A(KDM6A) |
| KDM6A | DQ062678 | Homo sapiens | lysine demethylase 6A(KDM6A) |
| KDM6A | NM_021140 | Homo sapiens | lysine demethylase 6A(KDM6A) |
| KDM6A | AK307691 | Homo sapiens | lysine demethylase 6A(KDM6A) |
| KDM6A | AB208795 | Homo sapiens | lysine demethylase 6A(KDM6A) |
| KDM6A | XM_017029782 | Homo sapiens | lysine demethylase 6A(KDM6A) |
| KDM6A | EU026291 | Homo sapiens | lysine demethylase 6A(KDM6A) |
| KDM6A | XM_017029783 | Homo sapiens | lysine demethylase 6A(KDM6A) |
| KDM6A | XM_011543971 | Homo sapiens | lysine demethylase 6A(KDM6A) |
| KDM6A | XM_011543970 | Homo sapiens | lysine demethylase 6A(KDM6A) |
| KDM6A | AI364446 | Homo sapiens | lysine demethylase 6A(KDM6A) |
| KDM6A | AK303430 | Homo sapiens | lysine demethylase 6A(KDM6A) |
| KDM6A | XM_011543973 | Homo sapiens | lysine demethylase 6A(KDM6A) |
| KDM6A | XM_011543972 | Homo sapiens | lysine demethylase 6A(KDM6A) |
| KDM6A | DC381812 | Homo sapiens | lysine demethylase 6A(KDM6A) |
| KDM6A | XM_011543975 | Homo sapiens | lysine demethylase 6A(KDM6A) |
| KDM6A | NM_001291415 | Homo sapiens | lysine demethylase 6A(KDM6A) |
| KDM6A | XM_011543974 | Homo sapiens | lysine demethylase 6A(KDM6A) |
| KDM6A | XM_017029784 | Homo sapiens | lysine demethylase 6A(KDM6A) |
| KDM6A | XM_017029785 | Homo sapiens | lysine demethylase 6A(KDM6A) |
| KDM6A | BX640694 | Homo sapiens | lysine demethylase 6A(KDM6A) |
| KDM6A | BC143272 | Homo sapiens | lysine demethylase 6A(KDM6A) |
| MYOG | BC053899 | Homo sapiens | myogenin(MYOG) |
| MYOG | BT007233 | Homo sapiens | myogenin(MYOG) |
| MYOG | X17651 | Homo sapiens | myogenin(MYOG) |
| MYOG | AW469592 | Homo sapiens | myogenin(MYOG) |
| MYOG | BF204429 | Homo sapiens | myogenin(MYOG) |
| MYOG | EU831793 | Homo sapiens | myogenin(MYOG) |
| MYOG | EU831870 | Homo sapiens | myogenin(MYOG) |
| MYOG | NM_002479 | Homo sapiens | myogenin(MYOG) |
| GRID2 | AK308611 | Homo sapiens | glutamate ionotropic receptor delta type subunit 2(GRID2) |
| GRID2 | XM_017008124 | Homo sapiens | glutamate ionotropic receptor delta type subunit 2(GRID2) |
| GRID2 | AW295056 | Homo sapiens | glutamate ionotropic receptor delta type subunit 2(GRID2) |
| GRID2 | XM_017008123 | Homo sapiens | glutamate ionotropic receptor delta type subunit 2(GRID2) |
| GRID2 | XM_017008122 | Homo sapiens | glutamate ionotropic receptor delta type subunit 2(GRID2) |
| GRID2 | XM_017008121 | Homo sapiens | glutamate ionotropic receptor delta type subunit 2(GRID2) |
| GRID2 | XM_017008120 | Homo sapiens | glutamate ionotropic receptor delta type subunit 2(GRID2) |
| GRID2 | AK310262 | Homo sapiens | glutamate ionotropic receptor delta type subunit 2(GRID2) |
| GRID2 | AK302355 | Homo sapiens | glutamate ionotropic receptor delta type subunit 2(GRID2) |
| GRID2 | XM_017008119 | Homo sapiens | glutamate ionotropic receptor delta type subunit 2(GRID2) |
| GRID2 | NM_001510 | Homo sapiens | glutamate ionotropic receptor delta type subunit 2(GRID2) |
| GRID2 | XM_017008118 | Homo sapiens | glutamate ionotropic receptor delta type subunit 2(GRID2) |
| GRID2 | XM_017008127 | Homo sapiens | glutamate ionotropic receptor delta type subunit 2(GRID2) |
| GRID2 | XM_017008126 | Homo sapiens | glutamate ionotropic receptor delta type subunit 2(GRID2) |
| GRID2 | XM_017008125 | Homo sapiens | glutamate ionotropic receptor delta type subunit 2(GRID2) |
| GRID2 | BC099653 | Homo sapiens | glutamate ionotropic receptor delta type subunit 2(GRID2) |
| GRID2 | BC099652 | Homo sapiens | glutamate ionotropic receptor delta type subunit 2(GRID2) |
| GRID2 | BC099654 | Homo sapiens | glutamate ionotropic receptor delta type subunit 2(GRID2) |
| GRID2 | NM_001286838 | Homo sapiens | glutamate ionotropic receptor delta type subunit 2(GRID2) |
| GRID2 | XM_011531893 | Homo sapiens | glutamate ionotropic receptor delta type subunit 2(GRID2) |
| GRID2 | XM_011531895 | Homo sapiens | glutamate ionotropic receptor delta type subunit 2(GRID2) |
| GRID2 | AF009014 | Homo sapiens | glutamate ionotropic receptor delta type subunit 2(GRID2) |
| GRID2 | XM_011531894 | Homo sapiens | glutamate ionotropic receptor delta type subunit 2(GRID2) |
| GRID2 | AB209318 | Homo sapiens | glutamate ionotropic receptor delta type subunit 2(GRID2) |
| ADGRV1 | XM_017009963 | Homo sapiens | adhesion G protein-coupled receptor V1(ADGRV1) |
| ADGRV1 | AL136541 | Homo sapiens | adhesion G protein-coupled receptor V1(ADGRV1) |
| ADGRV1 | XM_017009964 | Homo sapiens | adhesion G protein-coupled receptor V1(ADGRV1) |
| ADGRV1 | XM_017009965 | Homo sapiens | adhesion G protein-coupled receptor V1(ADGRV1) |
| ADGRV1 | XM_017009966 | Homo sapiens | adhesion G protein-coupled receptor V1(ADGRV1) |
| ADGRV1 | XM_017009967 | Homo sapiens | adhesion G protein-coupled receptor V1(ADGRV1) |
| ADGRV1 | XM_017009968 | Homo sapiens | adhesion G protein-coupled receptor V1(ADGRV1) |
| ADGRV1 | XM_017009969 | Homo sapiens | adhesion G protein-coupled receptor V1(ADGRV1) |
| ADGRV1 | AF055084 | Homo sapiens | adhesion G protein-coupled receptor V1(ADGRV1) |
| ADGRV1 | AF435925 | Homo sapiens | adhesion G protein-coupled receptor V1(ADGRV1) |
| ADGRV1 | BC034748 | Homo sapiens | adhesion G protein-coupled receptor V1(ADGRV1) |
| ADGRV1 | BX493741 | Homo sapiens | adhesion G protein-coupled receptor V1(ADGRV1) |
| ADGRV1 | AB014586 | Homo sapiens | adhesion G protein-coupled receptor V1(ADGRV1) |
| ADGRV1 | AB075823 | Homo sapiens | adhesion G protein-coupled receptor V1(ADGRV1) |
| ADGRV1 | XM_017009973 | Homo sapiens | adhesion G protein-coupled receptor V1(ADGRV1) |
| ADGRV1 | XM_017009974 | Homo sapiens | adhesion G protein-coupled receptor V1(ADGRV1) |
| ADGRV1 | DA503892 | Homo sapiens | adhesion G protein-coupled receptor V1(ADGRV1) |
| ADGRV1 | AL133041 | Homo sapiens | adhesion G protein-coupled receptor V1(ADGRV1) |
| ADGRV1 | XM_017009970 | Homo sapiens | adhesion G protein-coupled receptor V1(ADGRV1) |
| ADGRV1 | XM_017009971 | Homo sapiens | adhesion G protein-coupled receptor V1(ADGRV1) |
| ADGRV1 | XM_017009972 | Homo sapiens | adhesion G protein-coupled receptor V1(ADGRV1) |
| ADGRV1 | AK024416 | Homo sapiens | adhesion G protein-coupled receptor V1(ADGRV1) |
| ADGRV1 | BI758469 | Homo sapiens | adhesion G protein-coupled receptor V1(ADGRV1) |
| ADGRV1 | NR_003149 | Homo sapiens | adhesion G protein-coupled receptor V1(ADGRV1) |
| ADGRV1 | CD633594 | Homo sapiens | adhesion G protein-coupled receptor V1(ADGRV1) |
| ADGRV1 | NM_032119 | Homo sapiens | adhesion G protein-coupled receptor V1(ADGRV1) |
| ITGA4 | AJ504733 | Homo sapiens | integrin subunit alpha 4(ITGA4) |
| ITGA4 | X16983 | Homo sapiens | integrin subunit alpha 4(ITGA4) |
| ITGA4 | BC080190 | Homo sapiens | integrin subunit alpha 4(ITGA4) |
| ITGA4 | BC146277 | Homo sapiens | integrin subunit alpha 4(ITGA4) |
| ITGA4 | NM_000885 | Homo sapiens | integrin subunit alpha 4(ITGA4) |
| ITGA4 | AK311738 | Homo sapiens | integrin subunit alpha 4(ITGA4) |
| ITGA4 | AB208885 | Homo sapiens | integrin subunit alpha 4(ITGA4) |
| ITGA4 | AK303220 | Homo sapiens | integrin subunit alpha 4(ITGA4) |
| ITGA4 | AJ508356 | Homo sapiens | integrin subunit alpha 4(ITGA4) |
| ITGA4 | AK303478 | Homo sapiens | integrin subunit alpha 4(ITGA4) |
| ITGA4 | BU689519 | Homo sapiens | integrin subunit alpha 4(ITGA4) |
| ITGA4 | NM_001316312 | Homo sapiens | integrin subunit alpha 4(ITGA4) |
| ITGA4 | BC055419 | Homo sapiens | integrin subunit alpha 4(ITGA4) |
| ITGA4 | DB114726 | Homo sapiens | integrin subunit alpha 4(ITGA4) |
| ITGA4 | BC156712 | Homo sapiens | integrin subunit alpha 4(ITGA4) |
| ITGA4 | AJ510248 | Homo sapiens | integrin subunit alpha 4(ITGA4) |
| ITGA4 | AJ510247 | Homo sapiens | integrin subunit alpha 4(ITGA4) |
| ITGA4 | AJ510246 | Homo sapiens | integrin subunit alpha 4(ITGA4) |
| ITGA4 | AK310610 | Homo sapiens | integrin subunit alpha 4(ITGA4) |
| ITGA4 | AJ510249 | Homo sapiens | integrin subunit alpha 4(ITGA4) |
| ITGA4 | BC016671 | Homo sapiens | integrin subunit alpha 4(ITGA4) |
| ITGA4 | L12002 | Homo sapiens | integrin subunit alpha 4(ITGA4) |
| PAX3 | U02309 | Homo sapiens | paired box 3(PAX3) |
| PAX3 | S69370 | Homo sapiens | paired box 3(PAX3) |
| PAX3 | BC101299 | Homo sapiens | paired box 3(PAX3) |
| PAX3 | NM_181461 | Homo sapiens | paired box 3(PAX3) |
| PAX3 | NM_181460 | Homo sapiens | paired box 3(PAX3) |
| PAX3 | AI399816 | Homo sapiens | paired box 3(PAX3) |
| PAX3 | NM_000438 | Homo sapiens | paired box 3(PAX3) |
| PAX3 | L07483 | Homo sapiens | paired box 3(PAX3) |
| PAX3 | NM_013942 | Homo sapiens | paired box 3(PAX3) |
| PAX3 | S69369 | Homo sapiens | paired box 3(PAX3) |
| PAX3 | CA389778 | Homo sapiens | paired box 3(PAX3) |
| PAX3 | AK291278 | Homo sapiens | paired box 3(PAX3) |
| PAX3 | AY251280 | Homo sapiens | paired box 3(PAX3) |
| PAX3 | NM_181458 | Homo sapiens | paired box 3(PAX3) |
| PAX3 | NM_181457 | Homo sapiens | paired box 3(PAX3) |
| PAX3 | BC101302 | Homo sapiens | paired box 3(PAX3) |
| PAX3 | NM_181459 | Homo sapiens | paired box 3(PAX3) |
| PAX3 | BC063547 | Homo sapiens | paired box 3(PAX3) |
| PAX3 | BC101301 | Homo sapiens | paired box 3(PAX3) |
| PAX3 | BC101300 | Homo sapiens | paired box 3(PAX3) |
| PAX3 | EU446645 | Homo sapiens | paired box 3(PAX3) |
| PAX3 | BC114363 | Homo sapiens | paired box 3(PAX3) |
| PAX3 | DN989642 | Homo sapiens | paired box 3(PAX3) |
| PAX3 | NM_001127366 | Homo sapiens | paired box 3(PAX3) |
| PAX3 | AI382779 | Homo sapiens | paired box 3(PAX3) |
| PAX3 | AY251279 | Homo sapiens | paired box 3(PAX3) |
| PAX3 | DA758804 | Homo sapiens | paired box 3(PAX3) |
| NRG1 | XM_017013369 | Homo sapiens | neuregulin 1(NRG1) |
| NRG1 | U02329 | Homo sapiens | neuregulin 1(NRG1) |
| NRG1 | U02328 | Homo sapiens | neuregulin 1(NRG1) |
| NRG1 | U02327 | Homo sapiens | neuregulin 1(NRG1) |
| NRG1 | XM_011544512 | Homo sapiens | neuregulin 1(NRG1) |
| NRG1 | AF026146 | Homo sapiens | neuregulin 1(NRG1) |
| NRG1 | U02326 | Homo sapiens | neuregulin 1(NRG1) |
| NRG1 | BC007675 | Homo sapiens | neuregulin 1(NRG1) |
| NRG1 | BC064587 | Homo sapiens | neuregulin 1(NRG1) |
| NRG1 | BT007389 | Homo sapiens | neuregulin 1(NRG1) |
| NRG1 | DC425692 | Homo sapiens | neuregulin 1(NRG1) |
| NRG1 | NM_013964 | Homo sapiens | neuregulin 1(NRG1) |
| NRG1 | AF176921 | Homo sapiens | neuregulin 1(NRG1) |
| NRG1 | AK097005 | Homo sapiens | neuregulin 1(NRG1) |
| NRG1 | NM_013962 | Homo sapiens | neuregulin 1(NRG1) |
| NRG1 | NM_013960 | Homo sapiens | neuregulin 1(NRG1) |
| NRG1 | DA805080 | Homo sapiens | neuregulin 1(NRG1) |
| NRG1 | L12261 | Homo sapiens | neuregulin 1(NRG1) |
| NRG1 | XM_017013368 | Homo sapiens | neuregulin 1(NRG1) |
| NRG1 | XM_017013367 | Homo sapiens | neuregulin 1(NRG1) |
| NRG1 | U02330 | Homo sapiens | neuregulin 1(NRG1) |
| NRG1 | XM_017013366 | Homo sapiens | neuregulin 1(NRG1) |
| NRG1 | XM_017013365 | Homo sapiens | neuregulin 1(NRG1) |
| NRG1 | AK293240 | Homo sapiens | neuregulin 1(NRG1) |
| NRG1 | L12260 | Homo sapiens | neuregulin 1(NRG1) |
| NRG1 | AK298132 | Homo sapiens | neuregulin 1(NRG1) |
| NRG1 | EU359055 | Homo sapiens | neuregulin 1(NRG1) |
| NRG1 | AB451331 | Homo sapiens | neuregulin 1(NRG1) |
| NRG1 | AK223435 | Homo sapiens | neuregulin 1(NRG1) |
| NRG1 | EF372274 | Homo sapiens | neuregulin 1(NRG1) |
| NRG1 | M94165 | Homo sapiens | neuregulin 1(NRG1) |
| NRG1 | EF372275 | Homo sapiens | neuregulin 1(NRG1) |
| NRG1 | M94166 | Homo sapiens | neuregulin 1(NRG1) |
| NRG1 | M94167 | Homo sapiens | neuregulin 1(NRG1) |
| NRG1 | EF372273 | Homo sapiens | neuregulin 1(NRG1) |
| NRG1 | M94168 | Homo sapiens | neuregulin 1(NRG1) |
| NRG1 | BC006492 | Homo sapiens | neuregulin 1(NRG1) |
| NRG1 | EF372276 | Homo sapiens | neuregulin 1(NRG1) |
| NRG1 | EU363510 | Homo sapiens | neuregulin 1(NRG1) |
| NRG1 | EF372277 | Homo sapiens | neuregulin 1(NRG1) |
| NRG1 | GQ983557 | Homo sapiens | neuregulin 1(NRG1) |
| NRG1 | L41827 | Homo sapiens | neuregulin 1(NRG1) |
| NRG1 | GQ983558 | Homo sapiens | neuregulin 1(NRG1) |
| NRG1 | CN603661 | Homo sapiens | neuregulin 1(NRG1) |
| NRG1 | NM_004495 | Homo sapiens | neuregulin 1(NRG1) |
| NRG1 | NM_001159999 | Homo sapiens | neuregulin 1(NRG1) |
| NRG1 | NM_001322201 | Homo sapiens | neuregulin 1(NRG1) |
| NRG1 | NM_001322202 | Homo sapiens | neuregulin 1(NRG1) |
| NRG1 | NM_001322203 | Homo sapiens | neuregulin 1(NRG1) |
| NRG1 | NM_013959 | Homo sapiens | neuregulin 1(NRG1) |
| NRG1 | NM_001159995 | Homo sapiens | neuregulin 1(NRG1) |
| NRG1 | NM_001322205 | Homo sapiens | neuregulin 1(NRG1) |
| NRG1 | NM_013958 | Homo sapiens | neuregulin 1(NRG1) |
| NRG1 | AK290730 | Homo sapiens | neuregulin 1(NRG1) |
| NRG1 | NM_001159996 | Homo sapiens | neuregulin 1(NRG1) |
| NRG1 | NM_001322206 | Homo sapiens | neuregulin 1(NRG1) |
| NRG1 | NM_013957 | Homo sapiens | neuregulin 1(NRG1) |
| NRG1 | NM_001322207 | Homo sapiens | neuregulin 1(NRG1) |
| NRG1 | NM_013956 | Homo sapiens | neuregulin 1(NRG1) |
| NRG1 | CA429204 | Homo sapiens | neuregulin 1(NRG1) |
| NRG1 | AK293270 | Homo sapiens | neuregulin 1(NRG1) |
| NRG1 | EU363509 | Homo sapiens | neuregulin 1(NRG1) |
| NRG1 | NM_001322197 | Homo sapiens | neuregulin 1(NRG1) |
| NRG1 | BC144446 | Homo sapiens | neuregulin 1(NRG1) |
| NRG1 | EU363508 | Homo sapiens | neuregulin 1(NRG1) |
| NRG1 | XM_005273486 | Homo sapiens | neuregulin 1(NRG1) |
| NRG1 | BC073871 | Homo sapiens | neuregulin 1(NRG1) |
| NRG1 | CN603654 | Homo sapiens | neuregulin 1(NRG1) |
| NRG1 | CN603652 | Homo sapiens | neuregulin 1(NRG1) |
| NRG1 | EF517297 | Homo sapiens | neuregulin 1(NRG1) |
| NRG1 | XM_005273487 | Homo sapiens | neuregulin 1(NRG1) |
| NRG1 | AY207002 | Homo sapiens | neuregulin 1(NRG1) |
| NRG1 | EF517295 | Homo sapiens | neuregulin 1(NRG1) |
| NRG1 | EF517296 | Homo sapiens | neuregulin 1(NRG1) |
| NRG1 | CR450288 | Homo sapiens | neuregulin 1(NRG1) |
| NRG1 | BC150609 | Homo sapiens | neuregulin 1(NRG1) |
| NRG1 | HG501045 | Homo sapiens | neuregulin 1(NRG1) |
| NRG1 | NM_001160001 | Homo sapiens | neuregulin 1(NRG1) |
| NRG1 | AB451475 | Homo sapiens | neuregulin 1(NRG1) |
| NRG1 | NM_001160008 | Homo sapiens | neuregulin 1(NRG1) |
| NRG1 | NM_001160007 | Homo sapiens | neuregulin 1(NRG1) |
| NRG1 | S68256 | Homo sapiens | neuregulin 1(NRG1) |
| NRG1 | NM_001160004 | Homo sapiens | neuregulin 1(NRG1) |
| NRG1 | NM_001160005 | Homo sapiens | neuregulin 1(NRG1) |
| NRG1 | NM_001160002 | Homo sapiens | neuregulin 1(NRG1) |
| NRG1 | AK289927 | Homo sapiens | neuregulin 1(NRG1) |
| NRG1 | BQ018711 | Homo sapiens | neuregulin 1(NRG1) |
| NRG1 | XM_017013371 | Homo sapiens | neuregulin 1(NRG1) |
| NRG1 | XM_017013370 | Homo sapiens | neuregulin 1(NRG1) |
| NRG1 | U02325 | Homo sapiens | neuregulin 1(NRG1) |
| NRG1 | XM_006716335 | Homo sapiens | neuregulin 1(NRG1) |
| NRG1 | XM_017013372 | Homo sapiens | neuregulin 1(NRG1) |
| PAX6 | BX440968 | Homo sapiens | paired box 6(PAX6) |
| PAX6 | DQ894612 | Homo sapiens | paired box 6(PAX6) |
| PAX6 | AY047583 | Homo sapiens | paired box 6(PAX6) |
| PAX6 | CA397106 | Homo sapiens | paired box 6(PAX6) |
| PAX6 | CV569250 | Homo sapiens | paired box 6(PAX6) |
| PAX6 | BM557761 | Homo sapiens | paired box 6(PAX6) |
| PAX6 | KP255960 | Homo sapiens | paired box 6(PAX6) |
| PAX6 | AB593092 | Homo sapiens | paired box 6(PAX6) |
| PAX6 | AK094172 | Homo sapiens | paired box 6(PAX6) |
| PAX6 | AK074881 | Homo sapiens | paired box 6(PAX6) |
| PAX6 | BM725029 | Homo sapiens | paired box 6(PAX6) |
| PAX6 | AB209177 | Homo sapiens | paired box 6(PAX6) |
| PAX6 | AB593094 | Homo sapiens | paired box 6(PAX6) |
| PAX6 | AB593093 | Homo sapiens | paired box 6(PAX6) |
| PAX6 | AK314470 | Homo sapiens | paired box 6(PAX6) |
| PAX6 | DA183294 | Homo sapiens | paired box 6(PAX6) |
| PAX6 | NM_001127612 | Homo sapiens | paired box 6(PAX6) |
| PAX6 | DA141443 | Homo sapiens | paired box 6(PAX6) |
| PAX6 | BP394576 | Homo sapiens | paired box 6(PAX6) |
| PAX6 | DA089215 | Homo sapiens | paired box 6(PAX6) |
| PAX6 | CA397536 | Homo sapiens | paired box 6(PAX6) |
| PAX6 | NM_001310158 | Homo sapiens | paired box 6(PAX6) |
| PAX6 | BX114225 | Homo sapiens | paired box 6(PAX6) |
| PAX6 | M77844 | Homo sapiens | paired box 6(PAX6) |
| PAX6 | NM_001310159 | Homo sapiens | paired box 6(PAX6) |
| PAX6 | DA571138 | Homo sapiens | paired box 6(PAX6) |
| PAX6 | BM313099 | Homo sapiens | paired box 6(PAX6) |
| PAX6 | BE221553 | Homo sapiens | paired box 6(PAX6) |
| PAX6 | NM_001604 | Homo sapiens | paired box 6(PAX6) |
| PAX6 | BM666662 | Homo sapiens | paired box 6(PAX6) |
| PAX6 | BC011953 | Homo sapiens | paired box 6(PAX6) |
| PAX6 | BX089704 | Homo sapiens | paired box 6(PAX6) |
| PAX6 | NM_001258463 | Homo sapiens | paired box 6(PAX6) |
| PAX6 | NM_001258462 | Homo sapiens | paired box 6(PAX6) |
| PAX6 | NM_001258465 | Homo sapiens | paired box 6(PAX6) |
| PAX6 | NM_001258464 | Homo sapiens | paired box 6(PAX6) |
| PAX6 | BP394398 | Homo sapiens | paired box 6(PAX6) |
| PAX6 | DA079367 | Homo sapiens | paired box 6(PAX6) |
| PAX6 | NM_001310161 | Homo sapiens | paired box 6(PAX6) |
| PAX6 | GQ141695 | Homo sapiens | paired box 6(PAX6) |
| PAX6 | NM_001310160 | Homo sapiens | paired box 6(PAX6) |
| PAX6 | DA056636 | Homo sapiens | paired box 6(PAX6) |
| PAX6 | BU072567 | Homo sapiens | paired box 6(PAX6) |
| PAX6 | BX640762 | Homo sapiens | paired box 6(PAX6) |
| PAX6 | NM_000280 | Homo sapiens | paired box 6(PAX6) |
| PAX6 | BI789171 | Homo sapiens | paired box 6(PAX6) |
| PAX6 | AY707088 | Homo sapiens | paired box 6(PAX6) |
| PAX6 | F07809 | Homo sapiens | paired box 6(PAX6) |
| PAX6 | DA078958 | Homo sapiens | paired box 6(PAX6) |
| PAX6 | DQ891436 | Homo sapiens | paired box 6(PAX6) |
| PAX6 | BI816814 | Homo sapiens | paired box 6(PAX6) |
| PAX6 | M93650 | Homo sapiens | paired box 6(PAX6) |
| PAX6 | AK094249 | Homo sapiens | paired box 6(PAX6) |
| PAX6 | CD673930 | Homo sapiens | paired box 6(PAX6) |
| SYP | AK313030 | Homo sapiens | synaptophysin(SYP) |
| SYP | AK094376 | Homo sapiens | synaptophysin(SYP) |
| SYP | AK315953 | Homo sapiens | synaptophysin(SYP) |
| SYP | NM_003179 | Homo sapiens | synaptophysin(SYP) |
| SYP | BC064550 | Homo sapiens | synaptophysin(SYP) |
| SYP | BQ637168 | Homo sapiens | synaptophysin(SYP) |
| SYP | AK295524 | Homo sapiens | synaptophysin(SYP) |
| SYP | HQ448085 | Homo sapiens | synaptophysin(SYP) |
| SYP | X06389 | Homo sapiens | synaptophysin(SYP) |
| SYP | BC032385 | Homo sapiens | synaptophysin(SYP) |
| GFRA2 | XM_011544484 | Homo sapiens | GDNF family receptor alpha 2(GFRA2) |
| GFRA2 | BC041688 | Homo sapiens | GDNF family receptor alpha 2(GFRA2) |
| GFRA2 | AY326396 | Homo sapiens | GDNF family receptor alpha 2(GFRA2) |
| GFRA2 | U93703 | Homo sapiens | GDNF family receptor alpha 2(GFRA2) |
| GFRA2 | NM_001165038 | Homo sapiens | GDNF family receptor alpha 2(GFRA2) |
| GFRA2 | AF002700 | Homo sapiens | GDNF family receptor alpha 2(GFRA2) |
| GFRA2 | AY941828 | Homo sapiens | GDNF family receptor alpha 2(GFRA2) |
| GFRA2 | NM_001165039 | Homo sapiens | GDNF family receptor alpha 2(GFRA2) |
| GFRA2 | JF432396 | Homo sapiens | GDNF family receptor alpha 2(GFRA2) |
| GFRA2 | NM_001495 | Homo sapiens | GDNF family receptor alpha 2(GFRA2) |
| GFRA2 | U97145 | Homo sapiens | GDNF family receptor alpha 2(GFRA2) |
| GFRA2 | XM_006716327 | Homo sapiens | GDNF family receptor alpha 2(GFRA2) |
| GFRA2 | AK299623 | Homo sapiens | GDNF family receptor alpha 2(GFRA2) |
| SYN1 | NM_006950 | Homo sapiens | synapsin I(SYN1) |
| SYN1 | BC036711 | Homo sapiens | synapsin I(SYN1) |
| SYN1 | BC048799 | Homo sapiens | synapsin I(SYN1) |
| SYN1 | AL833961 | Homo sapiens | synapsin I(SYN1) |
| SYN1 | NM_133499 | Homo sapiens | synapsin I(SYN1) |
| SYN1 | AI929645 | Homo sapiens | synapsin I(SYN1) |
| PAX2 | BC148710 | Homo sapiens | paired box 2(PAX2) |
| PAX2 | BC141452 | Homo sapiens | paired box 2(PAX2) |
| PAX2 | NM_000278 | Homo sapiens | paired box 2(PAX2) |
| PAX2 | BJ989901 | Homo sapiens | paired box 2(PAX2) |
| PAX2 | BM671839 | Homo sapiens | paired box 2(PAX2) |
| PAX2 | M89470 | Homo sapiens | paired box 2(PAX2) |
| PAX2 | NM_003987 | Homo sapiens | paired box 2(PAX2) |
| PAX2 | L25597 | Homo sapiens | paired box 2(PAX2) |
| PAX2 | AY153484 | Homo sapiens | paired box 2(PAX2) |
| PAX2 | NM_003989 | Homo sapiens | paired box 2(PAX2) |
| PAX2 | AY153483 | Homo sapiens | paired box 2(PAX2) |
| PAX2 | NM_003988 | Homo sapiens | paired box 2(PAX2) |
| PAX2 | DB077633 | Homo sapiens | paired box 2(PAX2) |
| PAX2 | XM_017016284 | Homo sapiens | paired box 2(PAX2) |
| PAX2 | XM_017016283 | Homo sapiens | paired box 2(PAX2) |
| PAX2 | XM_017016286 | Homo sapiens | paired box 2(PAX2) |
| PAX2 | XM_017016285 | Homo sapiens | paired box 2(PAX2) |
| PAX2 | NM_003990 | Homo sapiens | paired box 2(PAX2) |
| PAX2 | XM_017016282 | Homo sapiens | paired box 2(PAX2) |
| PAX2 | XM_017016281 | Homo sapiens | paired box 2(PAX2) |
| PAX2 | XM_017016288 | Homo sapiens | paired box 2(PAX2) |
| PAX2 | XM_017016287 | Homo sapiens | paired box 2(PAX2) |
| PAX2 | NM_001304569 | Homo sapiens | paired box 2(PAX2) |
| PAX2 | XM_017016289 | Homo sapiens | paired box 2(PAX2) |
| PARD3B | AK056157 | Homo sapiens | par-3 family cell polarity regulator beta(PARD3B) |
| PARD3B | BC172499 | Homo sapiens | par-3 family cell polarity regulator beta(PARD3B) |
| PARD3B | XM_017003284 | Homo sapiens | par-3 family cell polarity regulator beta(PARD3B) |
| PARD3B | XM_017003283 | Homo sapiens | par-3 family cell polarity regulator beta(PARD3B) |
| PARD3B | BC012984 | Homo sapiens | par-3 family cell polarity regulator beta(PARD3B) |
| PARD3B | XM_017003289 | Homo sapiens | par-3 family cell polarity regulator beta(PARD3B) |
| PARD3B | XM_017003286 | Homo sapiens | par-3 family cell polarity regulator beta(PARD3B) |
| PARD3B | NM_205863 | Homo sapiens | par-3 family cell polarity regulator beta(PARD3B) |
| PARD3B | XM_017003285 | Homo sapiens | par-3 family cell polarity regulator beta(PARD3B) |
| PARD3B | AF428251 | Homo sapiens | par-3 family cell polarity regulator beta(PARD3B) |
| PARD3B | BC156252 | Homo sapiens | par-3 family cell polarity regulator beta(PARD3B) |
| PARD3B | NM_057177 | Homo sapiens | par-3 family cell polarity regulator beta(PARD3B) |
| PARD3B | XM_017003288 | Homo sapiens | par-3 family cell polarity regulator beta(PARD3B) |
| PARD3B | AF428250 | Homo sapiens | par-3 family cell polarity regulator beta(PARD3B) |
| PARD3B | NM_152526 | Homo sapiens | par-3 family cell polarity regulator beta(PARD3B) |
| PARD3B | XM_017003287 | Homo sapiens | par-3 family cell polarity regulator beta(PARD3B) |
| PARD3B | XM_011510553 | Homo sapiens | par-3 family cell polarity regulator beta(PARD3B) |
| PARD3B | AF466152 | Homo sapiens | par-3 family cell polarity regulator beta(PARD3B) |
| PARD3B | XM_011510552 | Homo sapiens | par-3 family cell polarity regulator beta(PARD3B) |
| PARD3B | NM_001302769 | Homo sapiens | par-3 family cell polarity regulator beta(PARD3B) |
| PARD3B | AK057965 | Homo sapiens | par-3 family cell polarity regulator beta(PARD3B) |
| PARD3B | AW274720 | Homo sapiens | par-3 family cell polarity regulator beta(PARD3B) |
| PARD3B | XM_017003293 | Homo sapiens | par-3 family cell polarity regulator beta(PARD3B) |
| PARD3B | BF434006 | Homo sapiens | par-3 family cell polarity regulator beta(PARD3B) |
| PARD3B | XM_017003292 | Homo sapiens | par-3 family cell polarity regulator beta(PARD3B) |
| PARD3B | XM_017003294 | Homo sapiens | par-3 family cell polarity regulator beta(PARD3B) |
| PARD3B | AI742400 | Homo sapiens | par-3 family cell polarity regulator beta(PARD3B) |
| PARD3B | XM_017003291 | Homo sapiens | par-3 family cell polarity regulator beta(PARD3B) |
| PARD3B | XM_017003290 | Homo sapiens | par-3 family cell polarity regulator beta(PARD3B) |
| PARD3B | AB073472 | Homo sapiens | par-3 family cell polarity regulator beta(PARD3B) |
| PARD3B | AK312083 | Homo sapiens | par-3 family cell polarity regulator beta(PARD3B) |
| PARD3B | AL832951 | Homo sapiens | par-3 family cell polarity regulator beta(PARD3B) |
| PARD3B | AB053321 | Homo sapiens | par-3 family cell polarity regulator beta(PARD3B) |
| PARD3B | AB092439 | Homo sapiens | par-3 family cell polarity regulator beta(PARD3B) |
| CCNA2 | BP363915 | Homo sapiens | cyclin A2(CCNA2) |
| CCNA2 | CR407692 | Homo sapiens | cyclin A2(CCNA2) |
| CCNA2 | AW276578 | Homo sapiens | cyclin A2(CCNA2) |
| CCNA2 | BC104783 | Homo sapiens | cyclin A2(CCNA2) |
| CCNA2 | AK291931 | Homo sapiens | cyclin A2(CCNA2) |
| CCNA2 | BC104787 | Homo sapiens | cyclin A2(CCNA2) |
| CCNA2 | NM_001237 | Homo sapiens | cyclin A2(CCNA2) |
| CCNA2 | X51688 | Homo sapiens | cyclin A2(CCNA2) |
| CCNA2 | DA748568 | Homo sapiens | cyclin A2(CCNA2) |
| DNAJC3 | BC047936 | Homo sapiens | DnaJ heat shock protein family (Hsp40) member C3(DNAJC3) |
| DNAJC3 | XM_011521104 | Homo sapiens | DnaJ heat shock protein family (Hsp40) member C3(DNAJC3) |
| DNAJC3 | AA430108 | Homo sapiens | DnaJ heat shock protein family (Hsp40) member C3(DNAJC3) |
| DNAJC3 | XM_011521105 | Homo sapiens | DnaJ heat shock protein family (Hsp40) member C3(DNAJC3) |
| DNAJC3 | AF339773 | Homo sapiens | DnaJ heat shock protein family (Hsp40) member C3(DNAJC3) |
| DNAJC3 | AK024941 | Homo sapiens | DnaJ heat shock protein family (Hsp40) member C3(DNAJC3) |
| DNAJC3 | XM_017020674 | Homo sapiens | DnaJ heat shock protein family (Hsp40) member C3(DNAJC3) |
| DNAJC3 | XM_017020675 | Homo sapiens | DnaJ heat shock protein family (Hsp40) member C3(DNAJC3) |
| DNAJC3 | BC033823 | Homo sapiens | DnaJ heat shock protein family (Hsp40) member C3(DNAJC3) |
| DNAJC3 | AF339835 | Homo sapiens | DnaJ heat shock protein family (Hsp40) member C3(DNAJC3) |
| DNAJC3 | AK292947 | Homo sapiens | DnaJ heat shock protein family (Hsp40) member C3(DNAJC3) |
| DNAJC3 | AK312861 | Homo sapiens | DnaJ heat shock protein family (Hsp40) member C3(DNAJC3) |
| DNAJC3 | NM_006260 | Homo sapiens | DnaJ heat shock protein family (Hsp40) member C3(DNAJC3) |
| ACTA1 | BC012597 | Homo sapiens | actin, alpha 1, skeletal muscle(ACTA1) |
| ACTA1 | CR541796 | Homo sapiens | actin, alpha 1, skeletal muscle(ACTA1) |
| ACTA1 | DQ896197 | Homo sapiens | actin, alpha 1, skeletal muscle(ACTA1) |
| ACTA1 | AY280960 | Homo sapiens | actin, alpha 1, skeletal muscle(ACTA1) |
| ACTA1 | AL598491 | Homo sapiens | actin, alpha 1, skeletal muscle(ACTA1) |
| ACTA1 | CR536516 | Homo sapiens | actin, alpha 1, skeletal muscle(ACTA1) |
| ACTA1 | DQ892949 | Homo sapiens | actin, alpha 1, skeletal muscle(ACTA1) |
| ACTA1 | NM_001100 | Homo sapiens | actin, alpha 1, skeletal muscle(ACTA1) |
| ACTA1 | J00068 | Homo sapiens | actin, alpha 1, skeletal muscle(ACTA1) |
| ACTA1 | AK300697 | Homo sapiens | actin, alpha 1, skeletal muscle(ACTA1) |
| ACTA1 | BX648545 | Homo sapiens | actin, alpha 1, skeletal muscle(ACTA1) |
| ACTA1 | AK096902 | Homo sapiens | actin, alpha 1, skeletal muscle(ACTA1) |
| ASPM | AY367065 | Homo sapiens | abnormal spindle microtubule assembly(ASPM) |
| ASPM | AY099892 | Homo sapiens | abnormal spindle microtubule assembly(ASPM) |
| ASPM | BC040439 | Homo sapiens | abnormal spindle microtubule assembly(ASPM) |
| ASPM | NM_018136 | Homo sapiens | abnormal spindle microtubule assembly(ASPM) |
| ASPM | AY099891 | Homo sapiens | abnormal spindle microtubule assembly(ASPM) |
| ASPM | AY971956 | Homo sapiens | abnormal spindle microtubule assembly(ASPM) |
| ASPM | AK001380 | Homo sapiens | abnormal spindle microtubule assembly(ASPM) |
| ASPM | AY099893 | Homo sapiens | abnormal spindle microtubule assembly(ASPM) |
| ASPM | AY971955 | Homo sapiens | abnormal spindle microtubule assembly(ASPM) |
| ASPM | AY099890 | Homo sapiens | abnormal spindle microtubule assembly(ASPM) |
| ASPM | NM_001206846 | Homo sapiens | abnormal spindle microtubule assembly(ASPM) |
| ASPM | AK095892 | Homo sapiens | abnormal spindle microtubule assembly(ASPM) |
| ASPM | BF970006 | Homo sapiens | abnormal spindle microtubule assembly(ASPM) |
| ASPM | AK001379 | Homo sapiens | abnormal spindle microtubule assembly(ASPM) |
| ASPM | AK226178 | Homo sapiens | abnormal spindle microtubule assembly(ASPM) |
| ASPM | AF509326 | Homo sapiens | abnormal spindle microtubule assembly(ASPM) |
| ASPM | AK001411 | Homo sapiens | abnormal spindle microtubule assembly(ASPM) |
| ASPM | BC034607 | Homo sapiens | abnormal spindle microtubule assembly(ASPM) |
| ASPM | AY101201 | Homo sapiens | abnormal spindle microtubule assembly(ASPM) |
| ASPM | BX648804 | Homo sapiens | abnormal spindle microtubule assembly(ASPM) |
| ASPM | AK125107 | Homo sapiens | abnormal spindle microtubule assembly(ASPM) |
| ASPM | AY971957 | Homo sapiens | abnormal spindle microtubule assembly(ASPM) |
| ASPM | BC015396 | Homo sapiens | abnormal spindle microtubule assembly(ASPM) |
| DCT | NM_001922 | Homo sapiens | dopachrome tautomerase(DCT) |
| DCT | NM_001322182 | Homo sapiens | dopachrome tautomerase(DCT) |
| DCT | NM_001322183 | Homo sapiens | dopachrome tautomerase(DCT) |
| DCT | DQ891466 | Homo sapiens | dopachrome tautomerase(DCT) |
| DCT | NM_001322184 | Homo sapiens | dopachrome tautomerase(DCT) |
| DCT | NM_001322185 | Homo sapiens | dopachrome tautomerase(DCT) |
| DCT | XM_011521049 | Homo sapiens | dopachrome tautomerase(DCT) |
| DCT | NM_001322186 | Homo sapiens | dopachrome tautomerase(DCT) |
| DCT | DQ894649 | Homo sapiens | dopachrome tautomerase(DCT) |
| DCT | DQ902581 | Homo sapiens | dopachrome tautomerase(DCT) |
| DCT | AJ000503 | Homo sapiens | dopachrome tautomerase(DCT) |
| DCT | AJ132932 | Homo sapiens | dopachrome tautomerase(DCT) |
| DCT | BC028311 | Homo sapiens | dopachrome tautomerase(DCT) |
| DCT | AJ132933 | Homo sapiens | dopachrome tautomerase(DCT) |
| DCT | S69231 | Homo sapiens | dopachrome tautomerase(DCT) |
| DCT | NM_001129889 | Homo sapiens | dopachrome tautomerase(DCT) |
| DCT | XM_017020401 | Homo sapiens | dopachrome tautomerase(DCT) |
| DCT | BC104476 | Homo sapiens | dopachrome tautomerase(DCT) |
| DCT | BP221841 | Homo sapiens | dopachrome tautomerase(DCT) |
| DCT | L18967 | Homo sapiens | dopachrome tautomerase(DCT) |
| DCT | D17547 | Homo sapiens | dopachrome tautomerase(DCT) |
| DCT | AF339808 | Homo sapiens | dopachrome tautomerase(DCT) |
| DCT | AK293115 | Homo sapiens | dopachrome tautomerase(DCT) |
| PAX7 | DQ322591 | Homo sapiens | paired box 7(PAX7) |
| PAX7 | NM_001135254 | Homo sapiens | paired box 7(PAX7) |
| PAX7 | BC121165 | Homo sapiens | paired box 7(PAX7) |
| PAX7 | BC121166 | Homo sapiens | paired box 7(PAX7) |
| PAX7 | X96743 | Homo sapiens | paired box 7(PAX7) |
| PAX7 | NM_002584 | Homo sapiens | paired box 7(PAX7) |
| PAX7 | AY578141 | Homo sapiens | paired box 7(PAX7) |
| PAX7 | Z35141 | Homo sapiens | paired box 7(PAX7) |
| PAX7 | AK094779 | Homo sapiens | paired box 7(PAX7) |
| PAX7 | DB474828 | Homo sapiens | paired box 7(PAX7) |
| PAX7 | NM_013945 | Homo sapiens | paired box 7(PAX7) |
| KIFC1 | AK297378 | Homo sapiens | kinesin family member C1(KIFC1) |
| KIFC1 | BC063567 | Homo sapiens | kinesin family member C1(KIFC1) |
| KIFC1 | D14678 | Homo sapiens | kinesin family member C1(KIFC1) |
| KIFC1 | XM_017010836 | Homo sapiens | kinesin family member C1(KIFC1) |
| KIFC1 | XM_017010837 | Homo sapiens | kinesin family member C1(KIFC1) |
| KIFC1 | AK303239 | Homo sapiens | kinesin family member C1(KIFC1) |
| KIFC1 | AI990093 | Homo sapiens | kinesin family member C1(KIFC1) |
| KIFC1 | NM_002263 | Homo sapiens | kinesin family member C1(KIFC1) |
| KIFC1 | XM_011514587 | Homo sapiens | kinesin family member C1(KIFC1) |
| KIFC1 | XM_011514585 | Homo sapiens | kinesin family member C1(KIFC1) |
| KIFC1 | BC073878 | Homo sapiens | kinesin family member C1(KIFC1) |
| KIFC1 | BC121041 | Homo sapiens | kinesin family member C1(KIFC1) |
| KIFC1 | AJ010479 | Homo sapiens | kinesin family member C1(KIFC1) |
| KIFC1 | BC000712 | Homo sapiens | kinesin family member C1(KIFC1) |
| KIFC1 | BC121042 | Homo sapiens | kinesin family member C1(KIFC1) |
| KIFC1 | BC098438 | Homo sapiens | kinesin family member C1(KIFC1) |
| KIFC1 | BE885900 | Homo sapiens | kinesin family member C1(KIFC1) |
| PLSCR5 | XM_017006373 | Homo sapiens | phospholipid scramblase family member 5(PLSCR5) |
| PLSCR5 | BC157886 | Homo sapiens | phospholipid scramblase family member 5(PLSCR5) |
| PLSCR5 | NM_001085420 | Homo sapiens | phospholipid scramblase family member 5(PLSCR5) |
| PLSCR5 | NM_001321245 | Homo sapiens | phospholipid scramblase family member 5(PLSCR5) |
| PLSCR5 | AY436642 | Homo sapiens | phospholipid scramblase family member 5(PLSCR5) |
| PLSCR5 | BX431427 | Homo sapiens | phospholipid scramblase family member 5(PLSCR5) |
| PLSCR5 | BC171917 | Homo sapiens | phospholipid scramblase family member 5(PLSCR5) |
| MYOD1 | AI300241 | Homo sapiens | myogenic differentiation 1(MYOD1) |
| MYOD1 | BT007157 | Homo sapiens | myogenic differentiation 1(MYOD1) |
| MYOD1 | X56677 | Homo sapiens | myogenic differentiation 1(MYOD1) |
| MYOD1 | BF304059 | Homo sapiens | myogenic differentiation 1(MYOD1) |
| MYOD1 | X17650 | Homo sapiens | myogenic differentiation 1(MYOD1) |
| MYOD1 | BC064493 | Homo sapiens | myogenic differentiation 1(MYOD1) |
| MYOD1 | BF205042 | Homo sapiens | myogenic differentiation 1(MYOD1) |
| MYOD1 | NM_002478 | Homo sapiens | myogenic differentiation 1(MYOD1) |
| MYOD1 | AK314968 | Homo sapiens | myogenic differentiation 1(MYOD1) |
| TNNT2 | AJ709280 | Homo sapiens | troponin T2, cardiac type(TNNT2) |
| TNNT2 | X74819 | Homo sapiens | troponin T2, cardiac type(TNNT2) |
| TNNT2 | L40162 | Homo sapiens | troponin T2, cardiac type(TNNT2) |
| TNNT2 | NM_001276345 | Homo sapiens | troponin T2, cardiac type(TNNT2) |
| TNNT2 | XM_011509946 | Homo sapiens | troponin T2, cardiac type(TNNT2) |
| TNNT2 | AL832707 | Homo sapiens | troponin T2, cardiac type(TNNT2) |
| TNNT2 | NM_001276346 | Homo sapiens | troponin T2, cardiac type(TNNT2) |
| TNNT2 | NM_001276347 | Homo sapiens | troponin T2, cardiac type(TNNT2) |
| TNNT2 | XM_011509944 | Homo sapiens | troponin T2, cardiac type(TNNT2) |
| TNNT2 | XM_011509943 | Homo sapiens | troponin T2, cardiac type(TNNT2) |
| TNNT2 | BC002653 | Homo sapiens | troponin T2, cardiac type(TNNT2) |
| TNNT2 | X79855 | Homo sapiens | troponin T2, cardiac type(TNNT2) |
| TNNT2 | X79856 | Homo sapiens | troponin T2, cardiac type(TNNT2) |
| TNNT2 | X83744 | Homo sapiens | troponin T2, cardiac type(TNNT2) |
| TNNT2 | X83743 | Homo sapiens | troponin T2, cardiac type(TNNT2) |
| TNNT2 | XM_017002216 | Homo sapiens | troponin T2, cardiac type(TNNT2) |
| TNNT2 | HQ447413 | Homo sapiens | troponin T2, cardiac type(TNNT2) |
| TNNT2 | XM_017002217 | Homo sapiens | troponin T2, cardiac type(TNNT2) |
| TNNT2 | AK309493 | Homo sapiens | troponin T2, cardiac type(TNNT2) |
| TNNT2 | NM_001001430 | Homo sapiens | troponin T2, cardiac type(TNNT2) |
| TNNT2 | AK125236 | Homo sapiens | troponin T2, cardiac type(TNNT2) |
| TNNT2 | AK290621 | Homo sapiens | troponin T2, cardiac type(TNNT2) |
| TNNT2 | AY277394 | Homo sapiens | troponin T2, cardiac type(TNNT2) |
| TNNT2 | NM_001001432 | Homo sapiens | troponin T2, cardiac type(TNNT2) |
| TNNT2 | NM_001001431 | Homo sapiens | troponin T2, cardiac type(TNNT2) |
| TNNT2 | AK055533 | Homo sapiens | troponin T2, cardiac type(TNNT2) |
| TNNT2 | AA865519 | Homo sapiens | troponin T2, cardiac type(TNNT2) |
| TNNT2 | NM_000364 | Homo sapiens | troponin T2, cardiac type(TNNT2) |
| TNNT2 | S71125 | Homo sapiens | troponin T2, cardiac type(TNNT2) |
| TNNT2 | S71126 | Homo sapiens | troponin T2, cardiac type(TNNT2) |
| TNNT2 | XM_011509939 | Homo sapiens | troponin T2, cardiac type(TNNT2) |
| TNNT2 | X79858 | Homo sapiens | troponin T2, cardiac type(TNNT2) |
| TNNT2 | XM_011509938 | Homo sapiens | troponin T2, cardiac type(TNNT2) |
| TNNT2 | X79859 | Homo sapiens | troponin T2, cardiac type(TNNT2) |
| TNNT2 | XM_006711509 | Homo sapiens | troponin T2, cardiac type(TNNT2) |
| TNNT2 | XM_006711508 | Homo sapiens | troponin T2, cardiac type(TNNT2) |
| TNNT2 | S64668 | Homo sapiens | troponin T2, cardiac type(TNNT2) |
| TNNT2 | XM_011509942 | Homo sapiens | troponin T2, cardiac type(TNNT2) |
| TNNT2 | X79861 | Homo sapiens | troponin T2, cardiac type(TNNT2) |
| TNNT2 | XM_011509941 | Homo sapiens | troponin T2, cardiac type(TNNT2) |
| TNNT2 | XM_011509940 | Homo sapiens | troponin T2, cardiac type(TNNT2) |
| DCX | AK299822 | Homo sapiens | doublecortin(DCX) |
| DCX | NM_000555 | Homo sapiens | doublecortin(DCX) |
| DCX | AK002120 | Homo sapiens | doublecortin(DCX) |
| DCX | AK297297 | Homo sapiens | doublecortin(DCX) |
| DCX | AU120758 | Homo sapiens | doublecortin(DCX) |
| DCX | XM_017029312 | Homo sapiens | doublecortin(DCX) |
| DCX | DC375659 | Homo sapiens | doublecortin(DCX) |
| DCX | HQ447825 | Homo sapiens | doublecortin(DCX) |
| DCX | NM_001195553 | Homo sapiens | doublecortin(DCX) |
| DCX | XM_011530880 | Homo sapiens | doublecortin(DCX) |
| DCX | AF040254 | Homo sapiens | doublecortin(DCX) |
| DCX | AJ003112 | Homo sapiens | doublecortin(DCX) |
| DCX | AF040255 | Homo sapiens | doublecortin(DCX) |
| DCX | NM_178151 | Homo sapiens | doublecortin(DCX) |
| DCX | AW163329 | Homo sapiens | doublecortin(DCX) |
| DCX | BC027925 | Homo sapiens | doublecortin(DCX) |
| DCX | AF034634 | Homo sapiens | doublecortin(DCX) |
| DCX | DA497601 | Homo sapiens | doublecortin(DCX) |
| DCX | XM_011530878 | Homo sapiens | doublecortin(DCX) |
| DCX | AK290455 | Homo sapiens | doublecortin(DCX) |
| DCX | NM_178153 | Homo sapiens | doublecortin(DCX) |
| DCX | NM_178152 | Homo sapiens | doublecortin(DCX) |
| DCX | XM_011530879 | Homo sapiens | doublecortin(DCX) |
| PMP2 | AK311758 | Homo sapiens | peripheral myelin protein 2(PMP2) |
| PMP2 | X62167 | Homo sapiens | peripheral myelin protein 2(PMP2) |
| PMP2 | BC034997 | Homo sapiens | peripheral myelin protein 2(PMP2) |
| PMP2 | CF455669 | Homo sapiens | peripheral myelin protein 2(PMP2) |
| PMP2 | CR541649 | Homo sapiens | peripheral myelin protein 2(PMP2) |
| PMP2 | DQ891506 | Homo sapiens | peripheral myelin protein 2(PMP2) |
| PMP2 | CR541738 | Homo sapiens | peripheral myelin protein 2(PMP2) |
| PMP2 | DQ891507 | Homo sapiens | peripheral myelin protein 2(PMP2) |
| PMP2 | NM_002677 | Homo sapiens | peripheral myelin protein 2(PMP2) |
| PMP2 | AK307118 | Homo sapiens | peripheral myelin protein 2(PMP2) |
| PMP2 | DQ894697 | Homo sapiens | peripheral myelin protein 2(PMP2) |
| PMP2 | DQ786226 | Homo sapiens | peripheral myelin protein 2(PMP2) |
| MAPT | AK226139 | Homo sapiens | microtubule associated protein tau(MAPT) |
| MAPT | BC061892 | Homo sapiens | microtubule associated protein tau(MAPT) |
| MAPT | AK299658 | Homo sapiens | microtubule associated protein tau(MAPT) |
| MAPT | BI552187 | Homo sapiens | microtubule associated protein tau(MAPT) |
| MAPT | AW295014 | Homo sapiens | microtubule associated protein tau(MAPT) |
| MAPT | BC101936 | Homo sapiens | microtubule associated protein tau(MAPT) |
| MAPT | BN000503 | Homo sapiens | microtubule associated protein tau(MAPT) |
| MAPT | X14474 | Homo sapiens | microtubule associated protein tau(MAPT) |
| MAPT | AW161357 | Homo sapiens | microtubule associated protein tau(MAPT) |
| MAPT | BC098281 | Homo sapiens | microtubule associated protein tau(MAPT) |
| MAPT | FJ429137 | Homo sapiens | microtubule associated protein tau(MAPT) |
| MAPT | XM_005257370 | Homo sapiens | microtubule associated protein tau(MAPT) |
| MAPT | XM_005257371 | Homo sapiens | microtubule associated protein tau(MAPT) |
| MAPT | NM_001123067 | Homo sapiens | microtubule associated protein tau(MAPT) |
| MAPT | NM_001123066 | Homo sapiens | microtubule associated protein tau(MAPT) |
| MAPT | BC114504 | Homo sapiens | microtubule associated protein tau(MAPT) |
| MAPT | BT006772 | Homo sapiens | microtubule associated protein tau(MAPT) |
| MAPT | BC099721 | Homo sapiens | microtubule associated protein tau(MAPT) |
| MAPT | BC071830 | Homo sapiens | microtubule associated protein tau(MAPT) |
| MAPT | BC000558 | Homo sapiens | microtubule associated protein tau(MAPT) |
| MAPT | AK095802 | Homo sapiens | microtubule associated protein tau(MAPT) |
| MAPT | NM_016834 | Homo sapiens | microtubule associated protein tau(MAPT) |
| MAPT | AK055986 | Homo sapiens | microtubule associated protein tau(MAPT) |
| MAPT | BC114948 | Homo sapiens | microtubule associated protein tau(MAPT) |
| MAPT | NM_016835 | Homo sapiens | microtubule associated protein tau(MAPT) |
| MAPT | BC094805 | Homo sapiens | microtubule associated protein tau(MAPT) |
| MAPT | XM_005257366 | Homo sapiens | microtubule associated protein tau(MAPT) |
| MAPT | XM_005257367 | Homo sapiens | microtubule associated protein tau(MAPT) |
| MAPT | BC040444 | Homo sapiens | microtubule associated protein tau(MAPT) |
| MAPT | DR002467 | Homo sapiens | microtubule associated protein tau(MAPT) |
| MAPT | XM_005257364 | Homo sapiens | microtubule associated protein tau(MAPT) |
| MAPT | AK299704 | Homo sapiens | microtubule associated protein tau(MAPT) |
| MAPT | XM_005257365 | Homo sapiens | microtubule associated protein tau(MAPT) |
| MAPT | AF456477 | Homo sapiens | microtubule associated protein tau(MAPT) |
| MAPT | XM_005257368 | Homo sapiens | microtubule associated protein tau(MAPT) |
| MAPT | XM_005257369 | Homo sapiens | microtubule associated protein tau(MAPT) |
| MAPT | AB073354 | Homo sapiens | microtubule associated protein tau(MAPT) |
| MAPT | AY730549 | Homo sapiens | microtubule associated protein tau(MAPT) |
| MAPT | J03778 | Homo sapiens | microtubule associated protein tau(MAPT) |
| MAPT | NM_005910 | Homo sapiens | microtubule associated protein tau(MAPT) |
| MAPT | XM_005257362 | Homo sapiens | microtubule associated protein tau(MAPT) |
| MAPT | AY526356 | Homo sapiens | microtubule associated protein tau(MAPT) |
| MAPT | NM_001203251 | Homo sapiens | microtubule associated protein tau(MAPT) |
| MAPT | NM_001203252 | Homo sapiens | microtubule associated protein tau(MAPT) |
| MAPT | NM_016841 | Homo sapiens | microtubule associated protein tau(MAPT) |
| MAPT | DN996935 | Homo sapiens | microtubule associated protein tau(MAPT) |
| MAPT | BC032572 | Homo sapiens | microtubule associated protein tau(MAPT) |
| MAPT | M25298 | Homo sapiens | microtubule associated protein tau(MAPT) |
| CFAP54 | XM_011539081 | Homo sapiens | cilia and flagella associated protein 54(CFAP54) |
| CFAP54 | DN831445 | Homo sapiens | cilia and flagella associated protein 54(CFAP54) |
| CFAP54 | XM_011539084 | Homo sapiens | cilia and flagella associated protein 54(CFAP54) |
| CFAP54 | XM_011539083 | Homo sapiens | cilia and flagella associated protein 54(CFAP54) |
| CFAP54 | AK056076 | Homo sapiens | cilia and flagella associated protein 54(CFAP54) |
| CFAP54 | BC148603 | Homo sapiens | cilia and flagella associated protein 54(CFAP54) |
| CFAP54 | BC041426 | Homo sapiens | cilia and flagella associated protein 54(CFAP54) |
| CFAP54 | NM_001306084 | Homo sapiens | cilia and flagella associated protein 54(CFAP54) |
| CFAP54 | XR_945241 | Homo sapiens | cilia and flagella associated protein 54(CFAP54) |
| CFAP54 | XM_011539069 | Homo sapiens | cilia and flagella associated protein 54(CFAP54) |
| CFAP54 | XR_945242 | Homo sapiens | cilia and flagella associated protein 54(CFAP54) |
| CFAP54 | XR_945243 | Homo sapiens | cilia and flagella associated protein 54(CFAP54) |
| CFAP54 | AK307646 | Homo sapiens | cilia and flagella associated protein 54(CFAP54) |
| CFAP54 | XM_011539085 | Homo sapiens | cilia and flagella associated protein 54(CFAP54) |
| CFAP54 | AK126100 | Homo sapiens | cilia and flagella associated protein 54(CFAP54) |
| CFAP54 | BX644506 | Homo sapiens | cilia and flagella associated protein 54(CFAP54) |
| CFAP54 | AK091789 | Homo sapiens | cilia and flagella associated protein 54(CFAP54) |
| CFAP54 | DV080428 | Homo sapiens | cilia and flagella associated protein 54(CFAP54) |
| CFAP54 | DV080725 | Homo sapiens | cilia and flagella associated protein 54(CFAP54) |
| CFAP54 | XM_011539071 | Homo sapiens | cilia and flagella associated protein 54(CFAP54) |
| CFAP54 | XM_011539070 | Homo sapiens | cilia and flagella associated protein 54(CFAP54) |
| CFAP54 | DV080224 | Homo sapiens | cilia and flagella associated protein 54(CFAP54) |
| CFAP54 | XM_011539073 | Homo sapiens | cilia and flagella associated protein 54(CFAP54) |
| CFAP54 | XM_011539072 | Homo sapiens | cilia and flagella associated protein 54(CFAP54) |
| CFAP54 | CF887548 | Homo sapiens | cilia and flagella associated protein 54(CFAP54) |
| CFAP54 | XM_011539079 | Homo sapiens | cilia and flagella associated protein 54(CFAP54) |
| CFAP54 | DB225896 | Homo sapiens | cilia and flagella associated protein 54(CFAP54) |
| CFAP54 | XM_011539078 | Homo sapiens | cilia and flagella associated protein 54(CFAP54) |
| CFAP54 | BC153167 | Homo sapiens | cilia and flagella associated protein 54(CFAP54) |
| CFAP54 | BX281772 | Homo sapiens | cilia and flagella associated protein 54(CFAP54) |
| CFAP54 | XM_011539074 | Homo sapiens | cilia and flagella associated protein 54(CFAP54) |
| CFAP54 | XM_011539077 | Homo sapiens | cilia and flagella associated protein 54(CFAP54) |
| CFAP54 | XM_011539076 | Homo sapiens | cilia and flagella associated protein 54(CFAP54) |
| CFAP54 | CF891050 | Homo sapiens | cilia and flagella associated protein 54(CFAP54) |
| CFAP54 | EG328110 | Homo sapiens | cilia and flagella associated protein 54(CFAP54) |
| CFAP54 | XM_017018867 | Homo sapiens | cilia and flagella associated protein 54(CFAP54) |
| CFAP54 | XM_017018866 | Homo sapiens | cilia and flagella associated protein 54(CFAP54) |
| CFAP54 | NM_198520 | Homo sapiens | cilia and flagella associated protein 54(CFAP54) |
| UGT2B7 | DQ893657 | Homo sapiens | UDP glucuronosyltransferase family 2 member B7(UGT2B7) |
| UGT2B7 | DQ895814 | Homo sapiens | UDP glucuronosyltransferase family 2 member B7(UGT2B7) |
| UGT2B7 | XM_005265702 | Homo sapiens | UDP glucuronosyltransferase family 2 member B7(UGT2B7) |
| UGT2B7 | J05428 | Homo sapiens | UDP glucuronosyltransferase family 2 member B7(UGT2B7) |
| UGT2B7 | AW614236 | Homo sapiens | UDP glucuronosyltransferase family 2 member B7(UGT2B7) |
| UGT2B7 | S82485 | Homo sapiens | UDP glucuronosyltransferase family 2 member B7(UGT2B7) |
| UGT2B7 | AK223142 | Homo sapiens | UDP glucuronosyltransferase family 2 member B7(UGT2B7) |
| UGT2B7 | AK313190 | Homo sapiens | UDP glucuronosyltransferase family 2 member B7(UGT2B7) |
| UGT2B7 | XM_011532230 | Homo sapiens | UDP glucuronosyltransferase family 2 member B7(UGT2B7) |
| UGT2B7 | NM_001074 | Homo sapiens | UDP glucuronosyltransferase family 2 member B7(UGT2B7) |
| UGT2B7 | NM_001330719 | Homo sapiens | UDP glucuronosyltransferase family 2 member B7(UGT2B7) |
| UGT2B7 | BC030974 | Homo sapiens | UDP glucuronosyltransferase family 2 member B7(UGT2B7) |
| UGT2B7 | XM_011532229 | Homo sapiens | UDP glucuronosyltransferase family 2 member B7(UGT2B7) |
| RBM47 | XM_017008310 | Homo sapiens | RNA binding motif protein 47(RBM47) |
| RBM47 | CB124502 | Homo sapiens | RNA binding motif protein 47(RBM47) |
| RBM47 | AK025296 | Homo sapiens | RNA binding motif protein 47(RBM47) |
| RBM47 | HQ258329 | Homo sapiens | RNA binding motif protein 47(RBM47) |
| RBM47 | DA861151 | Homo sapiens | RNA binding motif protein 47(RBM47) |
| RBM47 | BC034402 | Homo sapiens | RNA binding motif protein 47(RBM47) |
| RBM47 | XM_011513708 | Homo sapiens | RNA binding motif protein 47(RBM47) |
| RBM47 | BX648404 | Homo sapiens | RNA binding motif protein 47(RBM47) |
| RBM47 | XM_011513707 | Homo sapiens | RNA binding motif protein 47(RBM47) |
| RBM47 | XM_011513704 | Homo sapiens | RNA binding motif protein 47(RBM47) |
| RBM47 | BC126261 | Homo sapiens | RNA binding motif protein 47(RBM47) |
| RBM47 | BP332501 | Homo sapiens | RNA binding motif protein 47(RBM47) |
| RBM47 | BM837005 | Homo sapiens | RNA binding motif protein 47(RBM47) |
| RBM47 | AK000280 | Homo sapiens | RNA binding motif protein 47(RBM47) |
| RBM47 | NM_019027 | Homo sapiens | RNA binding motif protein 47(RBM47) |
| RBM47 | AF262323 | Homo sapiens | RNA binding motif protein 47(RBM47) |
| RBM47 | AK304206 | Homo sapiens | RNA binding motif protein 47(RBM47) |
| RBM47 | XM_017008308 | Homo sapiens | RNA binding motif protein 47(RBM47) |
| RBM47 | BC143942 | Homo sapiens | RNA binding motif protein 47(RBM47) |
| RBM47 | XM_017008307 | Homo sapiens | RNA binding motif protein 47(RBM47) |
| RBM47 | AK057152 | Homo sapiens | RNA binding motif protein 47(RBM47) |
| RBM47 | XM_017008306 | Homo sapiens | RNA binding motif protein 47(RBM47) |
| RBM47 | XM_017008305 | Homo sapiens | RNA binding motif protein 47(RBM47) |
| RBM47 | XM_017008304 | Homo sapiens | RNA binding motif protein 47(RBM47) |
| RBM47 | AK024997 | Homo sapiens | RNA binding motif protein 47(RBM47) |
| RBM47 | XM_005248107 | Homo sapiens | RNA binding motif protein 47(RBM47) |
| RBM47 | XM_005248108 | Homo sapiens | RNA binding motif protein 47(RBM47) |
| RBM47 | XM_005248109 | Homo sapiens | RNA binding motif protein 47(RBM47) |
| RBM47 | XM_005248103 | Homo sapiens | RNA binding motif protein 47(RBM47) |
| RBM47 | NM_001098634 | Homo sapiens | RNA binding motif protein 47(RBM47) |
| RBM47 | XM_017008309 | Homo sapiens | RNA binding motif protein 47(RBM47) |
| RBM47 | BC071585 | Homo sapiens | RNA binding motif protein 47(RBM47) |
| DNAH7 | Z83801 | Homo sapiens | dynein axonemal heavy chain 7(DNAH7) |
| DNAH7 | NM_018897 | Homo sapiens | dynein axonemal heavy chain 7(DNAH7) |
| DNAH7 | XM_011511491 | Homo sapiens | dynein axonemal heavy chain 7(DNAH7) |
| DNAH7 | XM_011511490 | Homo sapiens | dynein axonemal heavy chain 7(DNAH7) |
| DNAH7 | XM_011511493 | Homo sapiens | dynein axonemal heavy chain 7(DNAH7) |
| DNAH7 | BP199399 | Homo sapiens | dynein axonemal heavy chain 7(DNAH7) |
| DNAH7 | XM_011511492 | Homo sapiens | dynein axonemal heavy chain 7(DNAH7) |
| DNAH7 | BX117789 | Homo sapiens | dynein axonemal heavy chain 7(DNAH7) |
| DNAH7 | XM_011511488 | Homo sapiens | dynein axonemal heavy chain 7(DNAH7) |
| DNAH7 | XM_011511487 | Homo sapiens | dynein axonemal heavy chain 7(DNAH7) |
| DNAH7 | BC029567 | Homo sapiens | dynein axonemal heavy chain 7(DNAH7) |
| DNAH7 | XM_011511489 | Homo sapiens | dynein axonemal heavy chain 7(DNAH7) |
| DNAH7 | AB023161 | Homo sapiens | dynein axonemal heavy chain 7(DNAH7) |
| DNAH7 | XM_011511495 | Homo sapiens | dynein axonemal heavy chain 7(DNAH7) |
| DNAH7 | XM_011511494 | Homo sapiens | dynein axonemal heavy chain 7(DNAH7) |
| DNAH7 | XR_922968 | Homo sapiens | dynein axonemal heavy chain 7(DNAH7) |
| DNAH7 | XM_011511497 | Homo sapiens | dynein axonemal heavy chain 7(DNAH7) |
| DNAH7 | XM_017004504 | Homo sapiens | dynein axonemal heavy chain 7(DNAH7) |
| DNAH7 | AJ132084 | Homo sapiens | dynein axonemal heavy chain 7(DNAH7) |
| DNAH7 | CR749651 | Homo sapiens | dynein axonemal heavy chain 7(DNAH7) |
| DNAH7 | AK094515 | Homo sapiens | dynein axonemal heavy chain 7(DNAH7) |
| DNAH7 | AF327442 | Homo sapiens | dynein axonemal heavy chain 7(DNAH7) |
| COL11A1 | J04177 | Homo sapiens | collagen type XI alpha 1 chain(COL11A1) |
| COL11A1 | AK299025 | Homo sapiens | collagen type XI alpha 1 chain(COL11A1) |
| COL11A1 | AU118365 | Homo sapiens | collagen type XI alpha 1 chain(COL11A1) |
| COL11A1 | BC117697 | Homo sapiens | collagen type XI alpha 1 chain(COL11A1) |
| COL11A1 | AB208844 | Homo sapiens | collagen type XI alpha 1 chain(COL11A1) |
| COL11A1 | NM_001190709 | Homo sapiens | collagen type XI alpha 1 chain(COL11A1) |
| COL11A1 | NM_080629 | Homo sapiens | collagen type XI alpha 1 chain(COL11A1) |
| COL11A1 | NM_001854 | Homo sapiens | collagen type XI alpha 1 chain(COL11A1) |
| COL11A1 | BU159588 | Homo sapiens | collagen type XI alpha 1 chain(COL11A1) |
| COL11A1 | NM_080630 | Homo sapiens | collagen type XI alpha 1 chain(COL11A1) |
| COL11A1 | BX476821 | Homo sapiens | collagen type XI alpha 1 chain(COL11A1) |
| COL11A1 | NR_134980 | Homo sapiens | collagen type XI alpha 1 chain(COL11A1) |
| COL11A1 | XM_017000334 | Homo sapiens | collagen type XI alpha 1 chain(COL11A1) |
| COL11A1 | XM_017000335 | Homo sapiens | collagen type XI alpha 1 chain(COL11A1) |
| COL11A1 | XM_017000336 | Homo sapiens | collagen type XI alpha 1 chain(COL11A1) |
| COL11A1 | CA448616 | Homo sapiens | collagen type XI alpha 1 chain(COL11A1) |
| COL11A1 | XM_017000337 | Homo sapiens | collagen type XI alpha 1 chain(COL11A1) |
| LAMA4 | AK027151 | Homo sapiens | laminin subunit alpha 4(LAMA4) |
| LAMA4 | X76939 | Homo sapiens | laminin subunit alpha 4(LAMA4) |
| LAMA4 | U77706 | Homo sapiens | laminin subunit alpha 4(LAMA4) |
| LAMA4 | AB210027 | Homo sapiens | laminin subunit alpha 4(LAMA4) |
| LAMA4 | XM_017010854 | Homo sapiens | laminin subunit alpha 4(LAMA4) |
| LAMA4 | S78569 | Homo sapiens | laminin subunit alpha 4(LAMA4) |
| LAMA4 | BC004241 | Homo sapiens | laminin subunit alpha 4(LAMA4) |
| LAMA4 | BM662226 | Homo sapiens | laminin subunit alpha 4(LAMA4) |
| LAMA4 | NM_001105207 | Homo sapiens | laminin subunit alpha 4(LAMA4) |
| LAMA4 | NM_001105208 | Homo sapiens | laminin subunit alpha 4(LAMA4) |
| LAMA4 | NM_001105209 | Homo sapiens | laminin subunit alpha 4(LAMA4) |
| LAMA4 | BT006690 | Homo sapiens | laminin subunit alpha 4(LAMA4) |
| LAMA4 | NM_001105206 | Homo sapiens | laminin subunit alpha 4(LAMA4) |
| LAMA4 | AJ710266 | Homo sapiens | laminin subunit alpha 4(LAMA4) |
| LAMA4 | XM_005266984 | Homo sapiens | laminin subunit alpha 4(LAMA4) |
| LAMA4 | XM_005266983 | Homo sapiens | laminin subunit alpha 4(LAMA4) |
| LAMA4 | BX648467 | Homo sapiens | laminin subunit alpha 4(LAMA4) |
| LAMA4 | X70904 | Homo sapiens | laminin subunit alpha 4(LAMA4) |
| LAMA4 | BP234809 | Homo sapiens | laminin subunit alpha 4(LAMA4) |
| LAMA4 | BC066552 | Homo sapiens | laminin subunit alpha 4(LAMA4) |
| LAMA4 | BM014298 | Homo sapiens | laminin subunit alpha 4(LAMA4) |
| LAMA4 | JF432726 | Homo sapiens | laminin subunit alpha 4(LAMA4) |
| LAMA4 | AK304401 | Homo sapiens | laminin subunit alpha 4(LAMA4) |
| LAMA4 | CR407622 | Homo sapiens | laminin subunit alpha 4(LAMA4) |
| LAMA4 | XR_001743406 | Homo sapiens | laminin subunit alpha 4(LAMA4) |
| LAMA4 | XR_001743407 | Homo sapiens | laminin subunit alpha 4(LAMA4) |
| LAMA4 | BC026237 | Homo sapiens | laminin subunit alpha 4(LAMA4) |
| LAMA4 | DB271101 | Homo sapiens | laminin subunit alpha 4(LAMA4) |
| LAMA4 | NM_002290 | Homo sapiens | laminin subunit alpha 4(LAMA4) |
| LAMA4 | X91171 | Homo sapiens | laminin subunit alpha 4(LAMA4) |
| NRXN1 | BM021375 | Homo sapiens | neurexin 1(NRXN1) |
| NRXN1 | NM_001330078 | Homo sapiens | neurexin 1(NRXN1) |
| NRXN1 | NM_001330079 | Homo sapiens | neurexin 1(NRXN1) |
| NRXN1 | NM_001330077 | Homo sapiens | neurexin 1(NRXN1) |
| NRXN1 | XM_011533180 | Homo sapiens | neurexin 1(NRXN1) |
| NRXN1 | XM_017005304 | Homo sapiens | neurexin 1(NRXN1) |
| NRXN1 | XM_017005305 | Homo sapiens | neurexin 1(NRXN1) |
| NRXN1 | XM_017005303 | Homo sapiens | neurexin 1(NRXN1) |
| NRXN1 | BC046631 | Homo sapiens | neurexin 1(NRXN1) |
| NRXN1 | XM_011533183 | Homo sapiens | neurexin 1(NRXN1) |
| NRXN1 | XM_011533177 | Homo sapiens | neurexin 1(NRXN1) |
| NRXN1 | XM_011533178 | Homo sapiens | neurexin 1(NRXN1) |
| NRXN1 | XM_011533175 | Homo sapiens | neurexin 1(NRXN1) |
| NRXN1 | XM_017005308 | Homo sapiens | neurexin 1(NRXN1) |
| NRXN1 | AF087975 | Homo sapiens | neurexin 1(NRXN1) |
| NRXN1 | XM_017005309 | Homo sapiens | neurexin 1(NRXN1) |
| NRXN1 | BX647616 | Homo sapiens | neurexin 1(NRXN1) |
| NRXN1 | XM_017005306 | Homo sapiens | neurexin 1(NRXN1) |
| NRXN1 | XM_017005307 | Homo sapiens | neurexin 1(NRXN1) |
| NRXN1 | NM_001320156 | Homo sapiens | neurexin 1(NRXN1) |
| NRXN1 | NM_001320157 | Homo sapiens | neurexin 1(NRXN1) |
| NRXN1 | XM_006712140 | Homo sapiens | neurexin 1(NRXN1) |
| NRXN1 | XM_017005330 | Homo sapiens | neurexin 1(NRXN1) |
| NRXN1 | AB011150 | Homo sapiens | neurexin 1(NRXN1) |
| NRXN1 | BX113737 | Homo sapiens | neurexin 1(NRXN1) |
| NRXN1 | EF539882 | Homo sapiens | neurexin 1(NRXN1) |
| NRXN1 | XM_017005337 | Homo sapiens | neurexin 1(NRXN1) |
| NRXN1 | XM_017005335 | Homo sapiens | neurexin 1(NRXN1) |
| NRXN1 | XM_017005336 | Homo sapiens | neurexin 1(NRXN1) |
| NRXN1 | NM_004801 | Homo sapiens | neurexin 1(NRXN1) |
| NRXN1 | XM_017005333 | Homo sapiens | neurexin 1(NRXN1) |
| NRXN1 | XM_011533174 | Homo sapiens | neurexin 1(NRXN1) |
| NRXN1 | XM_017005334 | Homo sapiens | neurexin 1(NRXN1) |
| NRXN1 | XM_011533171 | Homo sapiens | neurexin 1(NRXN1) |
| NRXN1 | XM_017005331 | Homo sapiens | neurexin 1(NRXN1) |
| NRXN1 | XM_011533172 | Homo sapiens | neurexin 1(NRXN1) |
| NRXN1 | XM_017005332 | Homo sapiens | neurexin 1(NRXN1) |
| NRXN1 | XM_011533167 | Homo sapiens | neurexin 1(NRXN1) |
| NRXN1 | AK295773 | Homo sapiens | neurexin 1(NRXN1) |
| NRXN1 | DA153976 | Homo sapiens | neurexin 1(NRXN1) |
| NRXN1 | AK124726 | Homo sapiens | neurexin 1(NRXN1) |
| NRXN1 | XM_005264643 | Homo sapiens | neurexin 1(NRXN1) |
| NRXN1 | BC150247 | Homo sapiens | neurexin 1(NRXN1) |
| NRXN1 | XM_017005326 | Homo sapiens | neurexin 1(NRXN1) |
| NRXN1 | NM_138735 | Homo sapiens | neurexin 1(NRXN1) |
| NRXN1 | XM_017005327 | Homo sapiens | neurexin 1(NRXN1) |
| NRXN1 | DA168874 | Homo sapiens | neurexin 1(NRXN1) |
| NRXN1 | XM_017005324 | Homo sapiens | neurexin 1(NRXN1) |
| NRXN1 | XM_017005325 | Homo sapiens | neurexin 1(NRXN1) |
| NRXN1 | XM_017005322 | Homo sapiens | neurexin 1(NRXN1) |
| NRXN1 | XM_017005323 | Homo sapiens | neurexin 1(NRXN1) |
| NRXN1 | XM_017005320 | Homo sapiens | neurexin 1(NRXN1) |
| NRXN1 | AK093260 | Homo sapiens | neurexin 1(NRXN1) |
| NRXN1 | XM_017005321 | Homo sapiens | neurexin 1(NRXN1) |
| NRXN1 | C15866 | Homo sapiens | neurexin 1(NRXN1) |
| NRXN1 | NM_001135659 | Homo sapiens | neurexin 1(NRXN1) |
| NRXN1 | XM_017005328 | Homo sapiens | neurexin 1(NRXN1) |
| NRXN1 | DC324848 | Homo sapiens | neurexin 1(NRXN1) |
| NRXN1 | XM_017005329 | Homo sapiens | neurexin 1(NRXN1) |
| NRXN1 | DA320230 | Homo sapiens | neurexin 1(NRXN1) |
| NRXN1 | NM_001330096 | Homo sapiens | neurexin 1(NRXN1) |
| NRXN1 | DA322531 | Homo sapiens | neurexin 1(NRXN1) |
| NRXN1 | NM_001330097 | Homo sapiens | neurexin 1(NRXN1) |
| NRXN1 | XM_005264642 | Homo sapiens | neurexin 1(NRXN1) |
| NRXN1 | NM_001330094 | Homo sapiens | neurexin 1(NRXN1) |
| NRXN1 | NM_001330095 | Homo sapiens | neurexin 1(NRXN1) |
| NRXN1 | NM_001330092 | Homo sapiens | neurexin 1(NRXN1) |
| NRXN1 | NM_001330093 | Homo sapiens | neurexin 1(NRXN1) |
| NRXN1 | NM_001330090 | Homo sapiens | neurexin 1(NRXN1) |
| NRXN1 | NM_001330091 | Homo sapiens | neurexin 1(NRXN1) |
| NRXN1 | NM_001330089 | Homo sapiens | neurexin 1(NRXN1) |
| NRXN1 | BC125179 | Homo sapiens | neurexin 1(NRXN1) |
| NRXN1 | NM_001330087 | Homo sapiens | neurexin 1(NRXN1) |
| NRXN1 | NM_001330088 | Homo sapiens | neurexin 1(NRXN1) |
| NRXN1 | XM_017005315 | Homo sapiens | neurexin 1(NRXN1) |
| NRXN1 | XM_017005316 | Homo sapiens | neurexin 1(NRXN1) |
| NRXN1 | XM_017005313 | Homo sapiens | neurexin 1(NRXN1) |
| NRXN1 | XM_017005314 | Homo sapiens | neurexin 1(NRXN1) |
| NRXN1 | XM_017005311 | Homo sapiens | neurexin 1(NRXN1) |
| NRXN1 | XM_017005312 | Homo sapiens | neurexin 1(NRXN1) |
| NRXN1 | XM_017005310 | Homo sapiens | neurexin 1(NRXN1) |
| NRXN1 | BC125180 | Homo sapiens | neurexin 1(NRXN1) |
| NRXN1 | AB035356 | Homo sapiens | neurexin 1(NRXN1) |
| NRXN1 | XM_006712137 | Homo sapiens | neurexin 1(NRXN1) |
| NRXN1 | XM_017005319 | Homo sapiens | neurexin 1(NRXN1) |
| NRXN1 | XM_017005317 | Homo sapiens | neurexin 1(NRXN1) |
| NRXN1 | AF064842 | Homo sapiens | neurexin 1(NRXN1) |
| NRXN1 | XM_017005318 | Homo sapiens | neurexin 1(NRXN1) |
| NRXN1 | NM_001330085 | Homo sapiens | neurexin 1(NRXN1) |
| NRXN1 | NM_001330086 | Homo sapiens | neurexin 1(NRXN1) |
| NRXN1 | DC333548 | Homo sapiens | neurexin 1(NRXN1) |
| NRXN1 | NM_001330083 | Homo sapiens | neurexin 1(NRXN1) |
| NRXN1 | NM_001330084 | Homo sapiens | neurexin 1(NRXN1) |
| NRXN1 | NM_001330081 | Homo sapiens | neurexin 1(NRXN1) |
| NRXN1 | NM_001330082 | Homo sapiens | neurexin 1(NRXN1) |
| TYRP1 | DA278582 | Homo sapiens | tyrosinase related protein 1(TYRP1) |
| TYRP1 | CR407683 | Homo sapiens | tyrosinase related protein 1(TYRP1) |
| TYRP1 | X51420 | Homo sapiens | tyrosinase related protein 1(TYRP1) |
| TYRP1 | CD679533 | Homo sapiens | tyrosinase related protein 1(TYRP1) |
| TYRP1 | XR_001746372 | Homo sapiens | tyrosinase related protein 1(TYRP1) |
| TYRP1 | NM_000550 | Homo sapiens | tyrosinase related protein 1(TYRP1) |
| TYRP1 | BC052608 | Homo sapiens | tyrosinase related protein 1(TYRP1) |
| TYRP1 | AL600654 | Homo sapiens | tyrosinase related protein 1(TYRP1) |
| TYRP1 | X51455 | Homo sapiens | tyrosinase related protein 1(TYRP1) |
| TYRP1 | AK297887 | Homo sapiens | tyrosinase related protein 1(TYRP1) |
| NRXN3 | AK310235 | Homo sapiens | neurexin 3(NRXN3) |
| NRXN3 | AK056530 | Homo sapiens | neurexin 3(NRXN3) |
| NRXN3 | NM_001272020 | Homo sapiens | neurexin 3(NRXN3) |
| NRXN3 | XM_017021790 | Homo sapiens | neurexin 3(NRXN3) |
| NRXN3 | BC068469 | Homo sapiens | neurexin 3(NRXN3) |
| NRXN3 | DA243312 | Homo sapiens | neurexin 3(NRXN3) |
| NRXN3 | XM_017021792 | Homo sapiens | neurexin 3(NRXN3) |
| NRXN3 | BC142649 | Homo sapiens | neurexin 3(NRXN3) |
| NRXN3 | XM_017021791 | Homo sapiens | neurexin 3(NRXN3) |
| NRXN3 | XM_017021794 | Homo sapiens | neurexin 3(NRXN3) |
| NRXN3 | XM_017021793 | Homo sapiens | neurexin 3(NRXN3) |
| NRXN3 | XM_017021796 | Homo sapiens | neurexin 3(NRXN3) |
| NRXN3 | XM_017021795 | Homo sapiens | neurexin 3(NRXN3) |
| NRXN3 | XM_017021798 | Homo sapiens | neurexin 3(NRXN3) |
| NRXN3 | XM_017021797 | Homo sapiens | neurexin 3(NRXN3) |
| NRXN3 | XM_011537377 | Homo sapiens | neurexin 3(NRXN3) |
| NRXN3 | XM_017021799 | Homo sapiens | neurexin 3(NRXN3) |
| NRXN3 | NM_001330195 | Homo sapiens | neurexin 3(NRXN3) |
| NRXN3 | XM_006720323 | Homo sapiens | neurexin 3(NRXN3) |
| NRXN3 | AJ493127 | Homo sapiens | neurexin 3(NRXN3) |
| NRXN3 | XM_006720322 | Homo sapiens | neurexin 3(NRXN3) |
| NRXN3 | XR_001750599 | Homo sapiens | neurexin 3(NRXN3) |
| NRXN3 | DA124460 | Homo sapiens | neurexin 3(NRXN3) |
| NRXN3 | XM_005268218 | Homo sapiens | neurexin 3(NRXN3) |
| NRXN3 | XM_011537373 | Homo sapiens | neurexin 3(NRXN3) |
| NRXN3 | NM_001105250 | Homo sapiens | neurexin 3(NRXN3) |
| NRXN3 | XM_011537370 | Homo sapiens | neurexin 3(NRXN3) |
| NRXN3 | XM_011537371 | Homo sapiens | neurexin 3(NRXN3) |
| NRXN3 | XM_011537372 | Homo sapiens | neurexin 3(NRXN3) |
| NRXN3 | XM_011537366 | Homo sapiens | neurexin 3(NRXN3) |
| NRXN3 | XM_017021800 | Homo sapiens | neurexin 3(NRXN3) |
| NRXN3 | XM_011537367 | Homo sapiens | neurexin 3(NRXN3) |
| NRXN3 | AK096247 | Homo sapiens | neurexin 3(NRXN3) |
| NRXN3 | NM_138970 | Homo sapiens | neurexin 3(NRXN3) |
| NRXN3 | NR_073547 | Homo sapiens | neurexin 3(NRXN3) |
| NRXN3 | XM_011537368 | Homo sapiens | neurexin 3(NRXN3) |
| NRXN3 | XM_017021802 | Homo sapiens | neurexin 3(NRXN3) |
| NRXN3 | NR_073546 | Homo sapiens | neurexin 3(NRXN3) |
| NRXN3 | XM_011537369 | Homo sapiens | neurexin 3(NRXN3) |
| NRXN3 | XM_017021801 | Homo sapiens | neurexin 3(NRXN3) |
| NRXN3 | XM_017021804 | Homo sapiens | neurexin 3(NRXN3) |
| NRXN3 | XM_017021803 | Homo sapiens | neurexin 3(NRXN3) |
| NRXN3 | XM_017021806 | Homo sapiens | neurexin 3(NRXN3) |
| NRXN3 | XM_017021805 | Homo sapiens | neurexin 3(NRXN3) |
| NRXN3 | DA083962 | Homo sapiens | neurexin 3(NRXN3) |
| NRXN3 | XM_017021807 | Homo sapiens | neurexin 3(NRXN3) |
| NRXN3 | NM_004796 | Homo sapiens | neurexin 3(NRXN3) |
| NRXN3 | XM_011537363 | Homo sapiens | neurexin 3(NRXN3) |
| NRXN3 | XM_011537364 | Homo sapiens | neurexin 3(NRXN3) |
| NRXN3 | XM_011537365 | Homo sapiens | neurexin 3(NRXN3) |
| NRXN3 | AL833739 | Homo sapiens | neurexin 3(NRXN3) |
| NRXN3 | XR_943563 | Homo sapiens | neurexin 3(NRXN3) |
| NRXN3 | DA213090 | Homo sapiens | neurexin 3(NRXN3) |
| NRXN3 | XR_943562 | Homo sapiens | neurexin 3(NRXN3) |
| NRXN3 | XR_943561 | Homo sapiens | neurexin 3(NRXN3) |
| NRXN3 | XR_001750609 | Homo sapiens | neurexin 3(NRXN3) |
| NRXN3 | XR_001750608 | Homo sapiens | neurexin 3(NRXN3) |
| NRXN3 | XR_001750605 | Homo sapiens | neurexin 3(NRXN3) |
| NRXN3 | XR_001750604 | Homo sapiens | neurexin 3(NRXN3) |
| NRXN3 | XR_001750607 | Homo sapiens | neurexin 3(NRXN3) |
| NRXN3 | XR_001750606 | Homo sapiens | neurexin 3(NRXN3) |
| NRXN3 | XR_001750601 | Homo sapiens | neurexin 3(NRXN3) |
| NRXN3 | XR_001750600 | Homo sapiens | neurexin 3(NRXN3) |
| NRXN3 | XR_001750603 | Homo sapiens | neurexin 3(NRXN3) |
| NRXN3 | BC059368 | Homo sapiens | neurexin 3(NRXN3) |
| NRXN3 | XR_001750602 | Homo sapiens | neurexin 3(NRXN3) |
| NRXN3 | DA128446 | Homo sapiens | neurexin 3(NRXN3) |
| NRXN3 | XR_001750610 | Homo sapiens | neurexin 3(NRXN3) |
| NRXN3 | AB018286 | Homo sapiens | neurexin 3(NRXN3) |
| NRXN3 | BC152457 | Homo sapiens | neurexin 3(NRXN3) |
| NRXN3 | BC150194 | Homo sapiens | neurexin 3(NRXN3) |
| NRXN3 | AJ316284 | Homo sapiens | neurexin 3(NRXN3) |
| NRXN3 | AK126795 | Homo sapiens | neurexin 3(NRXN3) |
| ASCL1 | CF454566 | Homo sapiens | achaete-scute family bHLH transcription factor 1(ASCL1) |
| ASCL1 | BC002341 | Homo sapiens | achaete-scute family bHLH transcription factor 1(ASCL1) |
| ASCL1 | L08424 | Homo sapiens | achaete-scute family bHLH transcription factor 1(ASCL1) |
| ASCL1 | NM_004316 | Homo sapiens | achaete-scute family bHLH transcription factor 1(ASCL1) |
| ASCL1 | BC004425 | Homo sapiens | achaete-scute family bHLH transcription factor 1(ASCL1) |
| ASCL1 | DA005289 | Homo sapiens | achaete-scute family bHLH transcription factor 1(ASCL1) |
| ASCL1 | BC003134 | Homo sapiens | achaete-scute family bHLH transcription factor 1(ASCL1) |
| ASCL1 | AW071836 | Homo sapiens | achaete-scute family bHLH transcription factor 1(ASCL1) |
| ASCL1 | EU176303 | Homo sapiens | achaete-scute family bHLH transcription factor 1(ASCL1) |
| ASCL1 | AK290539 | Homo sapiens | achaete-scute family bHLH transcription factor 1(ASCL1) |
| ASCL1 | BC001638 | Homo sapiens | achaete-scute family bHLH transcription factor 1(ASCL1) |
| ASCL1 | BC031299 | Homo sapiens | achaete-scute family bHLH transcription factor 1(ASCL1) |
| ASCL1 | AK314114 | Homo sapiens | achaete-scute family bHLH transcription factor 1(ASCL1) |
| ASCL1 | DQ894571 | Homo sapiens | achaete-scute family bHLH transcription factor 1(ASCL1) |
| ERBB3 | M34309 | Homo sapiens | erb-b2 receptor tyrosine kinase 3(ERBB3) |
| ERBB3 | AK295650 | Homo sapiens | erb-b2 receptor tyrosine kinase 3(ERBB3) |
| ERBB3 | U88360 | Homo sapiens | erb-b2 receptor tyrosine kinase 3(ERBB3) |
| ERBB3 | BM837872 | Homo sapiens | erb-b2 receptor tyrosine kinase 3(ERBB3) |
| ERBB3 | NM_001982 | Homo sapiens | erb-b2 receptor tyrosine kinase 3(ERBB3) |
| ERBB3 | BC082992 | Homo sapiens | erb-b2 receptor tyrosine kinase 3(ERBB3) |
| ERBB3 | BT007226 | Homo sapiens | erb-b2 receptor tyrosine kinase 3(ERBB3) |
| ERBB3 | NM_001005915 | Homo sapiens | erb-b2 receptor tyrosine kinase 3(ERBB3) |
| ERBB3 | BU674508 | Homo sapiens | erb-b2 receptor tyrosine kinase 3(ERBB3) |
| ERBB3 | U88359 | Homo sapiens | erb-b2 receptor tyrosine kinase 3(ERBB3) |
| ERBB3 | U88358 | Homo sapiens | erb-b2 receptor tyrosine kinase 3(ERBB3) |
| ERBB3 | U88357 | Homo sapiens | erb-b2 receptor tyrosine kinase 3(ERBB3) |
| ERBB3 | M29366 | Homo sapiens | erb-b2 receptor tyrosine kinase 3(ERBB3) |
| ERBB3 | BC041579 | Homo sapiens | erb-b2 receptor tyrosine kinase 3(ERBB3) |
| ERBB3 | AK291681 | Homo sapiens | erb-b2 receptor tyrosine kinase 3(ERBB3) |
| ERBB3 | AU100462 | Homo sapiens | erb-b2 receptor tyrosine kinase 3(ERBB3) |
| ERBB3 | DC347955 | Homo sapiens | erb-b2 receptor tyrosine kinase 3(ERBB3) |
| ERBB3 | AA524528 | Homo sapiens | erb-b2 receptor tyrosine kinase 3(ERBB3) |
| ERBB3 | AK294719 | Homo sapiens | erb-b2 receptor tyrosine kinase 3(ERBB3) |
| ERBB3 | DC344156 | Homo sapiens | erb-b2 receptor tyrosine kinase 3(ERBB3) |
| ERBB3 | BX641868 | Homo sapiens | erb-b2 receptor tyrosine kinase 3(ERBB3) |
| ERBB3 | AK300909 | Homo sapiens | erb-b2 receptor tyrosine kinase 3(ERBB3) |
| ERBB3 | AK125028 | Homo sapiens | erb-b2 receptor tyrosine kinase 3(ERBB3) |
| ERBB3 | AK124710 | Homo sapiens | erb-b2 receptor tyrosine kinase 3(ERBB3) |
| ERBB3 | S61953 | Homo sapiens | erb-b2 receptor tyrosine kinase 3(ERBB3) |
| ERBB3 | BC002706 | Homo sapiens | erb-b2 receptor tyrosine kinase 3(ERBB3) |
| ERBB4 | XM_017003581 | Homo sapiens | erb-b2 receptor tyrosine kinase 4(ERBB4) |
| ERBB4 | AB209697 | Homo sapiens | erb-b2 receptor tyrosine kinase 4(ERBB4) |
| ERBB4 | XM_017003580 | Homo sapiens | erb-b2 receptor tyrosine kinase 4(ERBB4) |
| ERBB4 | AI793060 | Homo sapiens | erb-b2 receptor tyrosine kinase 4(ERBB4) |
| ERBB4 | XM_017003582 | Homo sapiens | erb-b2 receptor tyrosine kinase 4(ERBB4) |
| ERBB4 | AK024204 | Homo sapiens | erb-b2 receptor tyrosine kinase 4(ERBB4) |
| ERBB4 | XM_005246377 | Homo sapiens | erb-b2 receptor tyrosine kinase 4(ERBB4) |
| ERBB4 | XM_005246376 | Homo sapiens | erb-b2 receptor tyrosine kinase 4(ERBB4) |
| ERBB4 | KT281867 | Homo sapiens | erb-b2 receptor tyrosine kinase 4(ERBB4) |
| ERBB4 | NM_005235 | Homo sapiens | erb-b2 receptor tyrosine kinase 4(ERBB4) |
| ERBB4 | XM_006712364 | Homo sapiens | erb-b2 receptor tyrosine kinase 4(ERBB4) |
| ERBB4 | AF007153 | Homo sapiens | erb-b2 receptor tyrosine kinase 4(ERBB4) |
| ERBB4 | BC143741 | Homo sapiens | erb-b2 receptor tyrosine kinase 4(ERBB4) |
| ERBB4 | CR627023 | Homo sapiens | erb-b2 receptor tyrosine kinase 4(ERBB4) |
| ERBB4 | L07868 | Homo sapiens | erb-b2 receptor tyrosine kinase 4(ERBB4) |
| ERBB4 | BC143747 | Homo sapiens | erb-b2 receptor tyrosine kinase 4(ERBB4) |
| ERBB4 | XM_017003578 | Homo sapiens | erb-b2 receptor tyrosine kinase 4(ERBB4) |
| ERBB4 | XM_017003577 | Homo sapiens | erb-b2 receptor tyrosine kinase 4(ERBB4) |
| ERBB4 | BC143749 | Homo sapiens | erb-b2 receptor tyrosine kinase 4(ERBB4) |
| ERBB4 | BQ015804 | Homo sapiens | erb-b2 receptor tyrosine kinase 4(ERBB4) |
| ERBB4 | AK308606 | Homo sapiens | erb-b2 receptor tyrosine kinase 4(ERBB4) |
| ERBB4 | AK126298 | Homo sapiens | erb-b2 receptor tyrosine kinase 4(ERBB4) |
| ERBB4 | KT310076 | Homo sapiens | erb-b2 receptor tyrosine kinase 4(ERBB4) |
| ERBB4 | XM_017003579 | Homo sapiens | erb-b2 receptor tyrosine kinase 4(ERBB4) |
| ERBB4 | BC112199 | Homo sapiens | erb-b2 receptor tyrosine kinase 4(ERBB4) |
| ERBB4 | NM_001042599 | Homo sapiens | erb-b2 receptor tyrosine kinase 4(ERBB4) |
| EPCAM | BC014785 | Homo sapiens | epithelial cell adhesion molecule(EPCAM) |
| EPCAM | CR542259 | Homo sapiens | epithelial cell adhesion molecule(EPCAM) |
| EPCAM | DA963872 | Homo sapiens | epithelial cell adhesion molecule(EPCAM) |
| EPCAM | M32325 | Homo sapiens | epithelial cell adhesion molecule(EPCAM) |
| EPCAM | M26481 | Homo sapiens | epithelial cell adhesion molecule(EPCAM) |
| EPCAM | M32306 | Homo sapiens | epithelial cell adhesion molecule(EPCAM) |
| EPCAM | DQ891338 | Homo sapiens | epithelial cell adhesion molecule(EPCAM) |
| EPCAM | M33011 | Homo sapiens | epithelial cell adhesion molecule(EPCAM) |
| EPCAM | NM_002354 | Homo sapiens | epithelial cell adhesion molecule(EPCAM) |
| EPCAM | AK026585 | Homo sapiens | epithelial cell adhesion molecule(EPCAM) |
| EPCAM | X14758 | Homo sapiens | epithelial cell adhesion molecule(EPCAM) |
| EPCAM | BP262680 | Homo sapiens | epithelial cell adhesion molecule(EPCAM) |
| EPCAM | AW050533 | Homo sapiens | epithelial cell adhesion molecule(EPCAM) |
| EPCAM | CR542283 | Homo sapiens | epithelial cell adhesion molecule(EPCAM) |
| EPCAM | DQ894520 | Homo sapiens | epithelial cell adhesion molecule(EPCAM) |
| EPCAM | DB044092 | Homo sapiens | epithelial cell adhesion molecule(EPCAM) |
| NCAM1 | AK054570 | Homo sapiens | neural cell adhesion molecule 1(NCAM1) |
| NCAM1 | AK056258 | Homo sapiens | neural cell adhesion molecule 1(NCAM1) |
| NCAM1 | BC014205 | Homo sapiens | neural cell adhesion molecule 1(NCAM1) |
| NCAM1 | U63041 | Homo sapiens | neural cell adhesion molecule 1(NCAM1) |
| NCAM1 | X59400 | Homo sapiens | neural cell adhesion molecule 1(NCAM1) |
| NCAM1 | GQ129418 | Homo sapiens | neural cell adhesion molecule 1(NCAM1) |
| NCAM1 | NM_000615 | Homo sapiens | neural cell adhesion molecule 1(NCAM1) |
| NCAM1 | BC047244 | Homo sapiens | neural cell adhesion molecule 1(NCAM1) |
| NCAM1 | BC029119 | Homo sapiens | neural cell adhesion molecule 1(NCAM1) |
| NCAM1 | X55322 | Homo sapiens | neural cell adhesion molecule 1(NCAM1) |
| NCAM1 | AK057509 | Homo sapiens | neural cell adhesion molecule 1(NCAM1) |
| NCAM1 | AW131780 | Homo sapiens | neural cell adhesion molecule 1(NCAM1) |
| NCAM1 | M17409 | Homo sapiens | neural cell adhesion molecule 1(NCAM1) |
| NCAM1 | AL832563 | Homo sapiens | neural cell adhesion molecule 1(NCAM1) |
| NCAM1 | AF005070 | Homo sapiens | neural cell adhesion molecule 1(NCAM1) |
| NCAM1 | AB209443 | Homo sapiens | neural cell adhesion molecule 1(NCAM1) |
| NCAM1 | NM_001076682 | Homo sapiens | neural cell adhesion molecule 1(NCAM1) |
| NCAM1 | S73101 | Homo sapiens | neural cell adhesion molecule 1(NCAM1) |
| NCAM1 | DA231540 | Homo sapiens | neural cell adhesion molecule 1(NCAM1) |
| NCAM1 | M22094 | Homo sapiens | neural cell adhesion molecule 1(NCAM1) |
| NCAM1 | AK303232 | Homo sapiens | neural cell adhesion molecule 1(NCAM1) |
| NCAM1 | BC019845 | Homo sapiens | neural cell adhesion molecule 1(NCAM1) |
| NCAM1 | AA029449 | Homo sapiens | neural cell adhesion molecule 1(NCAM1) |
| NCAM1 | NM_181351 | Homo sapiens | neural cell adhesion molecule 1(NCAM1) |
| NCAM1 | S71824 | Homo sapiens | neural cell adhesion molecule 1(NCAM1) |
| NCAM1 | EU832805 | Homo sapiens | neural cell adhesion molecule 1(NCAM1) |
| NCAM1 | NM_001242607 | Homo sapiens | neural cell adhesion molecule 1(NCAM1) |
| NCAM1 | X16841 | Homo sapiens | neural cell adhesion molecule 1(NCAM1) |
| NCAM1 | NM_001242608 | Homo sapiens | neural cell adhesion molecule 1(NCAM1) |
| NCAM1 | AK054929 | Homo sapiens | neural cell adhesion molecule 1(NCAM1) |
| NCAM1 | M17410 | Homo sapiens | neural cell adhesion molecule 1(NCAM1) |
| NCAM1 | DA337649 | Homo sapiens | neural cell adhesion molecule 1(NCAM1) |
| NCAM1 | AK314589 | Homo sapiens | neural cell adhesion molecule 1(NCAM1) |
| NCAM1 | AK292453 | Homo sapiens | neural cell adhesion molecule 1(NCAM1) |
| NCAM1 | AK299865 | Homo sapiens | neural cell adhesion molecule 1(NCAM1) |
| SLIT2 | AB017168 | Homo sapiens | slit guidance ligand 2(SLIT2) |
| SLIT2 | XM_017008845 | Homo sapiens | slit guidance ligand 2(SLIT2) |
| SLIT2 | AK308444 | Homo sapiens | slit guidance ligand 2(SLIT2) |
| SLIT2 | BI494498 | Homo sapiens | slit guidance ligand 2(SLIT2) |
| SLIT2 | XM_011513909 | Homo sapiens | slit guidance ligand 2(SLIT2) |
| SLIT2 | BC117190 | Homo sapiens | slit guidance ligand 2(SLIT2) |
| SLIT2 | BC143978 | Homo sapiens | slit guidance ligand 2(SLIT2) |
| SLIT2 | AF055585 | Homo sapiens | slit guidance ligand 2(SLIT2) |
| SLIT2 | BF112143 | Homo sapiens | slit guidance ligand 2(SLIT2) |
| SLIT2 | NM_004787 | Homo sapiens | slit guidance ligand 2(SLIT2) |
| SLIT2 | AK027326 | Homo sapiens | slit guidance ligand 2(SLIT2) |
| SLIT2 | DB207352 | Homo sapiens | slit guidance ligand 2(SLIT2) |
| SLIT2 | DA721496 | Homo sapiens | slit guidance ligand 2(SLIT2) |
| SLIT2 | BM474839 | Homo sapiens | slit guidance ligand 2(SLIT2) |
| SLIT2 | AA489463 | Homo sapiens | slit guidance ligand 2(SLIT2) |
| SLIT2 | XM_011513910 | Homo sapiens | slit guidance ligand 2(SLIT2) |
| SLIT2 | XM_006713986 | Homo sapiens | slit guidance ligand 2(SLIT2) |
| SLIT2 | CV370469 | Homo sapiens | slit guidance ligand 2(SLIT2) |
| SLIT2 | NM_001289136 | Homo sapiens | slit guidance ligand 2(SLIT2) |
| SLIT2 | AK126459 | Homo sapiens | slit guidance ligand 2(SLIT2) |
| SLIT2 | NM_001289135 | Homo sapiens | slit guidance ligand 2(SLIT2) |
| SLIT2 | AF133270 | Homo sapiens | slit guidance ligand 2(SLIT2) |
| SLIT2 | XM_005248211 | Homo sapiens | slit guidance ligand 2(SLIT2) |
| MUC6 | AY458429 | Homo sapiens | mucin 6, oligomeric mucus/gel-forming(MUC6) |
| MUC6 | AK092533 | Homo sapiens | mucin 6, oligomeric mucus/gel-forming(MUC6) |
| MUC6 | NM_005961 | Homo sapiens | mucin 6, oligomeric mucus/gel-forming(MUC6) |
| MUC6 | AK098503 | Homo sapiens | mucin 6, oligomeric mucus/gel-forming(MUC6) |
| MUC6 | AY312160 | Homo sapiens | mucin 6, oligomeric mucus/gel-forming(MUC6) |
| MUC6 | U97698 | Homo sapiens | mucin 6, oligomeric mucus/gel-forming(MUC6) |
| MUC6 | AK096772 | Homo sapiens | mucin 6, oligomeric mucus/gel-forming(MUC6) |
| TBX1 | AF012131 | Homo sapiens | T-box 1(TBX1) |
| TBX1 | XM_017028927 | Homo sapiens | T-box 1(TBX1) |
| TBX1 | AF012130 | Homo sapiens | T-box 1(TBX1) |
| TBX1 | XM_017028928 | Homo sapiens | T-box 1(TBX1) |
| TBX1 | XM_017028925 | Homo sapiens | T-box 1(TBX1) |
| TBX1 | NM_005992 | Homo sapiens | T-box 1(TBX1) |
| TBX1 | XM_017028926 | Homo sapiens | T-box 1(TBX1) |
| TBX1 | NM_080646 | Homo sapiens | T-box 1(TBX1) |
| TBX1 | AF373867 | Homo sapiens | T-box 1(TBX1) |
| TBX1 | NM_080647 | Homo sapiens | T-box 1(TBX1) |
| TBX1 | GU014843 | Homo sapiens | T-box 1(TBX1) |
| TBX1 | XM_006724312 | Homo sapiens | T-box 1(TBX1) |
| NTRK2 | AL713745 | Homo sapiens | neurotrophic receptor tyrosine kinase 2(NTRK2) |
| NTRK2 | XM_005252003 | Homo sapiens | neurotrophic receptor tyrosine kinase 2(NTRK2) |
| NTRK2 | AF410901 | Homo sapiens | neurotrophic receptor tyrosine kinase 2(NTRK2) |
| NTRK2 | XM_005252001 | Homo sapiens | neurotrophic receptor tyrosine kinase 2(NTRK2) |
| NTRK2 | AF410900 | Homo sapiens | neurotrophic receptor tyrosine kinase 2(NTRK2) |
| NTRK2 | NM_001291937 | Homo sapiens | neurotrophic receptor tyrosine kinase 2(NTRK2) |
| NTRK2 | XM_005252007 | Homo sapiens | neurotrophic receptor tyrosine kinase 2(NTRK2) |
| NTRK2 | XM_005252006 | Homo sapiens | neurotrophic receptor tyrosine kinase 2(NTRK2) |
| NTRK2 | XM_005252004 | Homo sapiens | neurotrophic receptor tyrosine kinase 2(NTRK2) |
| NTRK2 | HY311899 | Homo sapiens | neurotrophic receptor tyrosine kinase 2(NTRK2) |
| NTRK2 | AL833196 | Homo sapiens | neurotrophic receptor tyrosine kinase 2(NTRK2) |
| NTRK2 | S76473 | Homo sapiens | neurotrophic receptor tyrosine kinase 2(NTRK2) |
| NTRK2 | S76474 | Homo sapiens | neurotrophic receptor tyrosine kinase 2(NTRK2) |
| NTRK2 | U12140 | Homo sapiens | neurotrophic receptor tyrosine kinase 2(NTRK2) |
| NTRK2 | BX649001 | Homo sapiens | neurotrophic receptor tyrosine kinase 2(NTRK2) |
| NTRK2 | AK123824 | Homo sapiens | neurotrophic receptor tyrosine kinase 2(NTRK2) |
| NTRK2 | AF410898 | Homo sapiens | neurotrophic receptor tyrosine kinase 2(NTRK2) |
| NTRK2 | AF410899 | Homo sapiens | neurotrophic receptor tyrosine kinase 2(NTRK2) |
| NTRK2 | AF508964 | Homo sapiens | neurotrophic receptor tyrosine kinase 2(NTRK2) |
| NTRK2 | BC031309 | Homo sapiens | neurotrophic receptor tyrosine kinase 2(NTRK2) |
| NTRK2 | AK289904 | Homo sapiens | neurotrophic receptor tyrosine kinase 2(NTRK2) |
| NTRK2 | BC075804 | Homo sapiens | neurotrophic receptor tyrosine kinase 2(NTRK2) |
| NTRK2 | AL533181 | Homo sapiens | neurotrophic receptor tyrosine kinase 2(NTRK2) |
| NTRK2 | XM_017014760 | Homo sapiens | neurotrophic receptor tyrosine kinase 2(NTRK2) |
| NTRK2 | XM_017014761 | Homo sapiens | neurotrophic receptor tyrosine kinase 2(NTRK2) |
| NTRK2 | XM_017014759 | Homo sapiens | neurotrophic receptor tyrosine kinase 2(NTRK2) |
| NTRK2 | AK294285 | Homo sapiens | neurotrophic receptor tyrosine kinase 2(NTRK2) |
| NTRK2 | AJ420458 | Homo sapiens | neurotrophic receptor tyrosine kinase 2(NTRK2) |
| NTRK2 | XM_017014755 | Homo sapiens | neurotrophic receptor tyrosine kinase 2(NTRK2) |
| NTRK2 | DA283111 | Homo sapiens | neurotrophic receptor tyrosine kinase 2(NTRK2) |
| NTRK2 | XM_017014756 | Homo sapiens | neurotrophic receptor tyrosine kinase 2(NTRK2) |
| NTRK2 | XM_017014757 | Homo sapiens | neurotrophic receptor tyrosine kinase 2(NTRK2) |
| NTRK2 | AB209118 | Homo sapiens | neurotrophic receptor tyrosine kinase 2(NTRK2) |
| NTRK2 | XM_011518720 | Homo sapiens | neurotrophic receptor tyrosine kinase 2(NTRK2) |
| NTRK2 | XM_017014758 | Homo sapiens | neurotrophic receptor tyrosine kinase 2(NTRK2) |
| NTRK2 | BE466753 | Homo sapiens | neurotrophic receptor tyrosine kinase 2(NTRK2) |
| NTRK2 | BC031835 | Homo sapiens | neurotrophic receptor tyrosine kinase 2(NTRK2) |
| NTRK2 | XM_011518718 | Homo sapiens | neurotrophic receptor tyrosine kinase 2(NTRK2) |
| NTRK2 | BE148667 | Homo sapiens | neurotrophic receptor tyrosine kinase 2(NTRK2) |
| NTRK2 | NM_001018066 | Homo sapiens | neurotrophic receptor tyrosine kinase 2(NTRK2) |
| NTRK2 | NM_001018065 | Homo sapiens | neurotrophic receptor tyrosine kinase 2(NTRK2) |
| NTRK2 | NM_001018064 | Homo sapiens | neurotrophic receptor tyrosine kinase 2(NTRK2) |
| NTRK2 | NM_001007097 | Homo sapiens | neurotrophic receptor tyrosine kinase 2(NTRK2) |
| NTRK2 | XM_017014751 | Homo sapiens | neurotrophic receptor tyrosine kinase 2(NTRK2) |
| NTRK2 | NM_006180 | Homo sapiens | neurotrophic receptor tyrosine kinase 2(NTRK2) |
| NTRK2 | XM_017014752 | Homo sapiens | neurotrophic receptor tyrosine kinase 2(NTRK2) |
| NTRK2 | XM_017014753 | Homo sapiens | neurotrophic receptor tyrosine kinase 2(NTRK2) |
| NTRK2 | XM_017014754 | Homo sapiens | neurotrophic receptor tyrosine kinase 2(NTRK2) |
| NTRK2 | AF086101 | Homo sapiens | neurotrophic receptor tyrosine kinase 2(NTRK2) |
| NTRK2 | DA449367 | Homo sapiens | neurotrophic receptor tyrosine kinase 2(NTRK2) |
| NTRK2 | X75958 | Homo sapiens | neurotrophic receptor tyrosine kinase 2(NTRK2) |
| NTRK2 | AF400441 | Homo sapiens | neurotrophic receptor tyrosine kinase 2(NTRK2) |
| NTRK2 | AK092267 | Homo sapiens | neurotrophic receptor tyrosine kinase 2(NTRK2) |
| NTRK2 | KC855566 | Homo sapiens | neurotrophic receptor tyrosine kinase 2(NTRK2) |
| NTRK2 | AW235842 | Homo sapiens | neurotrophic receptor tyrosine kinase 2(NTRK2) |
| NOP58 | BC018805 | Homo sapiens | NOP58 ribonucleoprotein(NOP58) |
| NOP58 | AK307396 | Homo sapiens | NOP58 ribonucleoprotein(NOP58) |
| NOP58 | BP245255 | Homo sapiens | NOP58 ribonucleoprotein(NOP58) |
| NOP58 | AL117554 | Homo sapiens | NOP58 ribonucleoprotein(NOP58) |
| NOP58 | DQ890868 | Homo sapiens | NOP58 ribonucleoprotein(NOP58) |
| NOP58 | EU176490 | Homo sapiens | NOP58 ribonucleoprotein(NOP58) |
| NOP58 | AK300844 | Homo sapiens | NOP58 ribonucleoprotein(NOP58) |
| NOP58 | AK023975 | Homo sapiens | NOP58 ribonucleoprotein(NOP58) |
| NOP58 | AF263608 | Homo sapiens | NOP58 ribonucleoprotein(NOP58) |
| NOP58 | BC009306 | Homo sapiens | NOP58 ribonucleoprotein(NOP58) |
| NOP58 | DQ586402 | Homo sapiens | NOP58 ribonucleoprotein(NOP58) |
| NOP58 | BC001707 | Homo sapiens | NOP58 ribonucleoprotein(NOP58) |
| NOP58 | BC032592 | Homo sapiens | NOP58 ribonucleoprotein(NOP58) |
| NOP58 | AF161469 | Homo sapiens | NOP58 ribonucleoprotein(NOP58) |
| NOP58 | NM_015934 | Homo sapiens | NOP58 ribonucleoprotein(NOP58) |
| NOP58 | AF123534 | Homo sapiens | NOP58 ribonucleoprotein(NOP58) |
| CDKN2C | BE783451 | Homo sapiens | cyclin dependent kinase inhibitor 2C(CDKN2C) |
| CDKN2C | DQ891516 | Homo sapiens | cyclin dependent kinase inhibitor 2C(CDKN2C) |
| CDKN2C | NM_078626 | Homo sapiens | cyclin dependent kinase inhibitor 2C(CDKN2C) |
| CDKN2C | U17074 | Homo sapiens | cyclin dependent kinase inhibitor 2C(CDKN2C) |
| CDKN2C | DQ894707 | Homo sapiens | cyclin dependent kinase inhibitor 2C(CDKN2C) |
| CDKN2C | AK021794 | Homo sapiens | cyclin dependent kinase inhibitor 2C(CDKN2C) |
| CDKN2C | BC005041 | Homo sapiens | cyclin dependent kinase inhibitor 2C(CDKN2C) |
| CDKN2C | BE831176 | Homo sapiens | cyclin dependent kinase inhibitor 2C(CDKN2C) |
| CDKN2C | AK091170 | Homo sapiens | cyclin dependent kinase inhibitor 2C(CDKN2C) |
| CDKN2C | AF041248 | Homo sapiens | cyclin dependent kinase inhibitor 2C(CDKN2C) |
| CDKN2C | NM_001262 | Homo sapiens | cyclin dependent kinase inhibitor 2C(CDKN2C) |
| CDKN2C | BC000598 | Homo sapiens | cyclin dependent kinase inhibitor 2C(CDKN2C) |
| CDKN2C | CR450289 | Homo sapiens | cyclin dependent kinase inhibitor 2C(CDKN2C) |
| CDKN2C | BC016173 | Homo sapiens | cyclin dependent kinase inhibitor 2C(CDKN2C) |
| CDKN2C | BC017036 | Homo sapiens | cyclin dependent kinase inhibitor 2C(CDKN2C) |
| PRRX1 | AA452918 | Homo sapiens | paired related homeobox 1(PRRX1) |
| PRRX1 | BC074993 | Homo sapiens | paired related homeobox 1(PRRX1) |
| PRRX1 | EU446648 | Homo sapiens | paired related homeobox 1(PRRX1) |
| PRRX1 | AA758879 | Homo sapiens | paired related homeobox 1(PRRX1) |
| PRRX1 | BI039001 | Homo sapiens | paired related homeobox 1(PRRX1) |
| PRRX1 | XM_006711388 | Homo sapiens | paired related homeobox 1(PRRX1) |
| PRRX1 | AB451463 | Homo sapiens | paired related homeobox 1(PRRX1) |
| PRRX1 | AV750422 | Homo sapiens | paired related homeobox 1(PRRX1) |
| PRRX1 | NM_022716 | Homo sapiens | paired related homeobox 1(PRRX1) |
| PRRX1 | AK225968 | Homo sapiens | paired related homeobox 1(PRRX1) |
| PRRX1 | NM_006902 | Homo sapiens | paired related homeobox 1(PRRX1) |
| USP9X | BC063645 | Homo sapiens | ubiquitin specific peptidase 9, X-linked(USP9X) |
| USP9X | BE925861 | Homo sapiens | ubiquitin specific peptidase 9, X-linked(USP9X) |
| USP9X | DA854185 | Homo sapiens | ubiquitin specific peptidase 9, X-linked(USP9X) |
| USP9X | AB209666 | Homo sapiens | ubiquitin specific peptidase 9, X-linked(USP9X) |
| USP9X | AJ012078 | Homo sapiens | ubiquitin specific peptidase 9, X-linked(USP9X) |
| USP9X | CD619095 | Homo sapiens | ubiquitin specific peptidase 9, X-linked(USP9X) |
| USP9X | BX956755 | Homo sapiens | ubiquitin specific peptidase 9, X-linked(USP9X) |
| USP9X | AA261797 | Homo sapiens | ubiquitin specific peptidase 9, X-linked(USP9X) |
| USP9X | BC046205 | Homo sapiens | ubiquitin specific peptidase 9, X-linked(USP9X) |
| USP9X | AW183065 | Homo sapiens | ubiquitin specific peptidase 9, X-linked(USP9X) |
| USP9X | XM_005272676 | Homo sapiens | ubiquitin specific peptidase 9, X-linked(USP9X) |
| USP9X | XM_005272675 | Homo sapiens | ubiquitin specific peptidase 9, X-linked(USP9X) |
| USP9X | NM_001039591 | Homo sapiens | ubiquitin specific peptidase 9, X-linked(USP9X) |
| USP9X | NM_021906 | Homo sapiens | ubiquitin specific peptidase 9, X-linked(USP9X) |
| USP9X | AF070645 | Homo sapiens | ubiquitin specific peptidase 9, X-linked(USP9X) |
| USP9X | AK294828 | Homo sapiens | ubiquitin specific peptidase 9, X-linked(USP9X) |
| USP9X | NM_004652 | Homo sapiens | ubiquitin specific peptidase 9, X-linked(USP9X) |
| USP9X | HG512500 | Homo sapiens | ubiquitin specific peptidase 9, X-linked(USP9X) |
| USP9X | BC172429 | Homo sapiens | ubiquitin specific peptidase 9, X-linked(USP9X) |
| USP9X | BC014792 | Homo sapiens | ubiquitin specific peptidase 9, X-linked(USP9X) |
| USP9X | NM_001039590 | Homo sapiens | ubiquitin specific peptidase 9, X-linked(USP9X) |
| USP9X | BC054861 | Homo sapiens | ubiquitin specific peptidase 9, X-linked(USP9X) |
| USP9X | X98296 | Homo sapiens | ubiquitin specific peptidase 9, X-linked(USP9X) |
| NTRK3 | NM_001243101 | Homo sapiens | neurotrophic receptor tyrosine kinase 3(NTRK3) |
| NTRK3 | XM_017022241 | Homo sapiens | neurotrophic receptor tyrosine kinase 3(NTRK3) |
| NTRK3 | XM_017022240 | Homo sapiens | neurotrophic receptor tyrosine kinase 3(NTRK3) |
| NTRK3 | XM_017022243 | Homo sapiens | neurotrophic receptor tyrosine kinase 3(NTRK3) |
| NTRK3 | XM_017022242 | Homo sapiens | neurotrophic receptor tyrosine kinase 3(NTRK3) |
| NTRK3 | XM_011521638 | Homo sapiens | neurotrophic receptor tyrosine kinase 3(NTRK3) |
| NTRK3 | XM_017022245 | Homo sapiens | neurotrophic receptor tyrosine kinase 3(NTRK3) |
| NTRK3 | XM_011521637 | Homo sapiens | neurotrophic receptor tyrosine kinase 3(NTRK3) |
| NTRK3 | XM_017022244 | Homo sapiens | neurotrophic receptor tyrosine kinase 3(NTRK3) |
| NTRK3 | AY065844 | Homo sapiens | neurotrophic receptor tyrosine kinase 3(NTRK3) |
| NTRK3 | XM_017022247 | Homo sapiens | neurotrophic receptor tyrosine kinase 3(NTRK3) |
| NTRK3 | XM_017022246 | Homo sapiens | neurotrophic receptor tyrosine kinase 3(NTRK3) |
| NTRK3 | XM_017022249 | Homo sapiens | neurotrophic receptor tyrosine kinase 3(NTRK3) |
| NTRK3 | CD370531 | Homo sapiens | neurotrophic receptor tyrosine kinase 3(NTRK3) |
| NTRK3 | XM_017022248 | Homo sapiens | neurotrophic receptor tyrosine kinase 3(NTRK3) |
| NTRK3 | NM_001007156 | Homo sapiens | neurotrophic receptor tyrosine kinase 3(NTRK3) |
| NTRK3 | CK903553 | Homo sapiens | neurotrophic receptor tyrosine kinase 3(NTRK3) |
| NTRK3 | S76475 | Homo sapiens | neurotrophic receptor tyrosine kinase 3(NTRK3) |
| NTRK3 | S76476 | Homo sapiens | neurotrophic receptor tyrosine kinase 3(NTRK3) |
| NTRK3 | DQ323597 | Homo sapiens | neurotrophic receptor tyrosine kinase 3(NTRK3) |
| NTRK3 | NM_001012338 | Homo sapiens | neurotrophic receptor tyrosine kinase 3(NTRK3) |
| NTRK3 | AF058389 | Homo sapiens | neurotrophic receptor tyrosine kinase 3(NTRK3) |
| NTRK3 | XM_017022250 | Homo sapiens | neurotrophic receptor tyrosine kinase 3(NTRK3) |
| NTRK3 | HY003065 | Homo sapiens | neurotrophic receptor tyrosine kinase 3(NTRK3) |
| NTRK3 | AK297160 | Homo sapiens | neurotrophic receptor tyrosine kinase 3(NTRK3) |
| NTRK3 | XM_017022252 | Homo sapiens | neurotrophic receptor tyrosine kinase 3(NTRK3) |
| NTRK3 | XM_017022251 | Homo sapiens | neurotrophic receptor tyrosine kinase 3(NTRK3) |
| NTRK3 | XM_017022254 | Homo sapiens | neurotrophic receptor tyrosine kinase 3(NTRK3) |
| NTRK3 | XM_017022253 | Homo sapiens | neurotrophic receptor tyrosine kinase 3(NTRK3) |
| NTRK3 | AL134171 | Homo sapiens | neurotrophic receptor tyrosine kinase 3(NTRK3) |
| NTRK3 | BG741347 | Homo sapiens | neurotrophic receptor tyrosine kinase 3(NTRK3) |
| NTRK3 | AF058390 | Homo sapiens | neurotrophic receptor tyrosine kinase 3(NTRK3) |
| NTRK3 | BT007291 | Homo sapiens | neurotrophic receptor tyrosine kinase 3(NTRK3) |
| NTRK3 | BC013693 | Homo sapiens | neurotrophic receptor tyrosine kinase 3(NTRK3) |
| NTRK3 | U05012 | Homo sapiens | neurotrophic receptor tyrosine kinase 3(NTRK3) |
| NTRK3 | AI613045 | Homo sapiens | neurotrophic receptor tyrosine kinase 3(NTRK3) |
| NTRK3 | BI962878 | Homo sapiens | neurotrophic receptor tyrosine kinase 3(NTRK3) |
| NTRK3 | NM_002530 | Homo sapiens | neurotrophic receptor tyrosine kinase 3(NTRK3) |
| NTRK3 | AL109700 | Homo sapiens | neurotrophic receptor tyrosine kinase 3(NTRK3) |
| NTRK3 | XR_001751292 | Homo sapiens | neurotrophic receptor tyrosine kinase 3(NTRK3) |
| NTRK3 | XR_001751293 | Homo sapiens | neurotrophic receptor tyrosine kinase 3(NTRK3) |
| NTRK3 | XM_006720550 | Homo sapiens | neurotrophic receptor tyrosine kinase 3(NTRK3) |
| NTRK3 | KJ534906 | Homo sapiens | neurotrophic receptor tyrosine kinase 3(NTRK3) |
| NTRK3 | KJ534905 | Homo sapiens | neurotrophic receptor tyrosine kinase 3(NTRK3) |
| NTRK3 | NM_001320134 | Homo sapiens | neurotrophic receptor tyrosine kinase 3(NTRK3) |
| NTRK3 | KJ534904 | Homo sapiens | neurotrophic receptor tyrosine kinase 3(NTRK3) |
| NTRK3 | NM_001320135 | Homo sapiens | neurotrophic receptor tyrosine kinase 3(NTRK3) |
| NTRK3 | AK313646 | Homo sapiens | neurotrophic receptor tyrosine kinase 3(NTRK3) |
| NTRK3 | XM_006720543 | Homo sapiens | neurotrophic receptor tyrosine kinase 3(NTRK3) |
| NTRK3 | AF125808 | Homo sapiens | neurotrophic receptor tyrosine kinase 3(NTRK3) |
| NTRK3 | AK309695 | Homo sapiens | neurotrophic receptor tyrosine kinase 3(NTRK3) |
| NTRK3 | BC128249 | Homo sapiens | neurotrophic receptor tyrosine kinase 3(NTRK3) |
| NTRK3 | XM_006720545 | Homo sapiens | neurotrophic receptor tyrosine kinase 3(NTRK3) |
| NTRK3 | XM_006720544 | Homo sapiens | neurotrophic receptor tyrosine kinase 3(NTRK3) |
| NTRK3 | KJ535052 | Homo sapiens | neurotrophic receptor tyrosine kinase 3(NTRK3) |
| NTRK3 | BQ893817 | Homo sapiens | neurotrophic receptor tyrosine kinase 3(NTRK3) |
| NTRK3 | AK302504 | Homo sapiens | neurotrophic receptor tyrosine kinase 3(NTRK3) |
| NTRK3 | AF052184 | Homo sapiens | neurotrophic receptor tyrosine kinase 3(NTRK3) |
| NTRK3 | BM887782 | Homo sapiens | neurotrophic receptor tyrosine kinase 3(NTRK3) |
| NTRK3 | DA789502 | Homo sapiens | neurotrophic receptor tyrosine kinase 3(NTRK3) |
| NTRK3 | BM675026 | Homo sapiens | neurotrophic receptor tyrosine kinase 3(NTRK3) |
| NTRK3 | CB053605 | Homo sapiens | neurotrophic receptor tyrosine kinase 3(NTRK3) |
| NTRK3 | XM_006720549 | Homo sapiens | neurotrophic receptor tyrosine kinase 3(NTRK3) |
| NTRK3 | AK094929 | Homo sapiens | neurotrophic receptor tyrosine kinase 3(NTRK3) |
| NTRK3 | XM_006720548 | Homo sapiens | neurotrophic receptor tyrosine kinase 3(NTRK3) |
| VEGFC | CR541897 | Homo sapiens | vascular endothelial growth factor C(VEGFC) |
| VEGFC | X94216 | Homo sapiens | vascular endothelial growth factor C(VEGFC) |
| VEGFC | DA044965 | Homo sapiens | vascular endothelial growth factor C(VEGFC) |
| VEGFC | U58111 | Homo sapiens | vascular endothelial growth factor C(VEGFC) |
| VEGFC | NM_005429 | Homo sapiens | vascular endothelial growth factor C(VEGFC) |
| VEGFC | BF528469 | Homo sapiens | vascular endothelial growth factor C(VEGFC) |
| VEGFC | BC063685 | Homo sapiens | vascular endothelial growth factor C(VEGFC) |
| VEGFC | AW294939 | Homo sapiens | vascular endothelial growth factor C(VEGFC) |
| VEGFC | DQ894481 | Homo sapiens | vascular endothelial growth factor C(VEGFC) |
| VEGFC | DQ891162 | Homo sapiens | vascular endothelial growth factor C(VEGFC) |
| VEGFC | DQ896666 | Homo sapiens | vascular endothelial growth factor C(VEGFC) |
| VEGFC | U43142 | Homo sapiens | vascular endothelial growth factor C(VEGFC) |
| VEGFC | DQ893345 | Homo sapiens | vascular endothelial growth factor C(VEGFC) |
| VEGFC | BC035212 | Homo sapiens | vascular endothelial growth factor C(VEGFC) |
| VEGFC | AK313879 | Homo sapiens | vascular endothelial growth factor C(VEGFC) |
| KCNJ16 | DA636394 | Homo sapiens | potassium voltage-gated channel subfamily J member 16(KCNJ16) |
| KCNJ16 | XM_006721885 | Homo sapiens | potassium voltage-gated channel subfamily J member 16(KCNJ16) |
| KCNJ16 | NM_001270422 | Homo sapiens | potassium voltage-gated channel subfamily J member 16(KCNJ16) |
| KCNJ16 | XM_005257337 | Homo sapiens | potassium voltage-gated channel subfamily J member 16(KCNJ16) |
| KCNJ16 | AK225944 | Homo sapiens | potassium voltage-gated channel subfamily J member 16(KCNJ16) |
| KCNJ16 | XM_006721887 | Homo sapiens | potassium voltage-gated channel subfamily J member 16(KCNJ16) |
| KCNJ16 | XM_006721886 | Homo sapiens | potassium voltage-gated channel subfamily J member 16(KCNJ16) |
| KCNJ16 | NM_001291623 | Homo sapiens | potassium voltage-gated channel subfamily J member 16(KCNJ16) |
| KCNJ16 | NM_001291624 | Homo sapiens | potassium voltage-gated channel subfamily J member 16(KCNJ16) |
| KCNJ16 | NM_001291622 | Homo sapiens | potassium voltage-gated channel subfamily J member 16(KCNJ16) |
| KCNJ16 | AF153815 | Homo sapiens | potassium voltage-gated channel subfamily J member 16(KCNJ16) |
| KCNJ16 | AF153816 | Homo sapiens | potassium voltage-gated channel subfamily J member 16(KCNJ16) |
| KCNJ16 | AF153817 | Homo sapiens | potassium voltage-gated channel subfamily J member 16(KCNJ16) |
| KCNJ16 | DA071381 | Homo sapiens | potassium voltage-gated channel subfamily J member 16(KCNJ16) |
| KCNJ16 | NM_170742 | Homo sapiens | potassium voltage-gated channel subfamily J member 16(KCNJ16) |
| KCNJ16 | BC030961 | Homo sapiens | potassium voltage-gated channel subfamily J member 16(KCNJ16) |
| KCNJ16 | NM_170741 | Homo sapiens | potassium voltage-gated channel subfamily J member 16(KCNJ16) |
| KCNJ16 | BC033038 | Homo sapiens | potassium voltage-gated channel subfamily J member 16(KCNJ16) |
| KCNJ16 | DQ892471 | Homo sapiens | potassium voltage-gated channel subfamily J member 16(KCNJ16) |
| KCNJ16 | DA109745 | Homo sapiens | potassium voltage-gated channel subfamily J member 16(KCNJ16) |
| KCNJ16 | NM_018658 | Homo sapiens | potassium voltage-gated channel subfamily J member 16(KCNJ16) |
| KCNJ16 | XM_017024609 | Homo sapiens | potassium voltage-gated channel subfamily J member 16(KCNJ16) |
| KCNJ16 | AF179353 | Homo sapiens | potassium voltage-gated channel subfamily J member 16(KCNJ16) |
| KCNJ16 | DA128508 | Homo sapiens | potassium voltage-gated channel subfamily J member 16(KCNJ16) |
| KCNJ16 | XM_017024610 | Homo sapiens | potassium voltage-gated channel subfamily J member 16(KCNJ16) |
| KCNJ16 | XM_017024613 | Homo sapiens | potassium voltage-gated channel subfamily J member 16(KCNJ16) |
| KCNJ16 | XM_017024611 | Homo sapiens | potassium voltage-gated channel subfamily J member 16(KCNJ16) |
| KCNJ16 | AK290799 | Homo sapiens | potassium voltage-gated channel subfamily J member 16(KCNJ16) |
| KCNJ16 | DA627411 | Homo sapiens | potassium voltage-gated channel subfamily J member 16(KCNJ16) |
| KCNJ16 | XM_017024612 | Homo sapiens | potassium voltage-gated channel subfamily J member 16(KCNJ16) |
| KCNJ16 | NM_001291625 | Homo sapiens | potassium voltage-gated channel subfamily J member 16(KCNJ16) |
| KCNJ16 | XM_011524781 | Homo sapiens | potassium voltage-gated channel subfamily J member 16(KCNJ16) |
| KCNJ16 | DA628604 | Homo sapiens | potassium voltage-gated channel subfamily J member 16(KCNJ16) |
| S100B | KU178320 | Homo sapiens | S100 calcium binding protein B(S100B) |
| S100B | EU176695 | Homo sapiens | S100 calcium binding protein B(S100B) |
| S100B | BC001766 | Homo sapiens | S100 calcium binding protein B(S100B) |
| S100B | BC041935 | Homo sapiens | S100 calcium binding protein B(S100B) |
| S100B | XM_017028424 | Homo sapiens | S100 calcium binding protein B(S100B) |
| S100B | AA310307 | Homo sapiens | S100 calcium binding protein B(S100B) |
| S100B | DQ892491 | Homo sapiens | S100 calcium binding protein B(S100B) |
| S100B | EB386131 | Homo sapiens | S100 calcium binding protein B(S100B) |
| S100B | KU178319 | Homo sapiens | S100 calcium binding protein B(S100B) |
| S100B | CR542123 | Homo sapiens | S100 calcium binding protein B(S100B) |
| S100B | NM_006272 | Homo sapiens | S100 calcium binding protein B(S100B) |
| CFC1 | BC069508 | Homo sapiens | cripto, FRL-1, cryptic family 1(CFC1) |
| CFC1 | NM_001270421 | Homo sapiens | cripto, FRL-1, cryptic family 1(CFC1) |
| CFC1 | CA777249 | Homo sapiens | cripto, FRL-1, cryptic family 1(CFC1) |
| CFC1 | NM_001270420 | Homo sapiens | cripto, FRL-1, cryptic family 1(CFC1) |
| CFC1 | BC146897 | Homo sapiens | cripto, FRL-1, cryptic family 1(CFC1) |
| CFC1 | CA941621 | Homo sapiens | cripto, FRL-1, cryptic family 1(CFC1) |
| CFC1 | AK290094 | Homo sapiens | cripto, FRL-1, cryptic family 1(CFC1) |
| CFC1 | BC074826 | Homo sapiens | cripto, FRL-1, cryptic family 1(CFC1) |
| CFC1 | CA948246 | Homo sapiens | cripto, FRL-1, cryptic family 1(CFC1) |
| CFC1 | BC110080 | Homo sapiens | cripto, FRL-1, cryptic family 1(CFC1) |
| CFC1 | BI793242 | Homo sapiens | cripto, FRL-1, cryptic family 1(CFC1) |
| CFC1 | BM310214 | Homo sapiens | cripto, FRL-1, cryptic family 1(CFC1) |
| CFC1 | NM_032545 | Homo sapiens | cripto, FRL-1, cryptic family 1(CFC1) |
| CFC1 | BG654700 | Homo sapiens | cripto, FRL-1, cryptic family 1(CFC1) |
| CFC1 | XM_011511486 | Homo sapiens | cripto, FRL-1, cryptic family 1(CFC1) |
| CFC1 | AK315326 | Homo sapiens | cripto, FRL-1, cryptic family 1(CFC1) |
| CFC1 | BC074825 | Homo sapiens | cripto, FRL-1, cryptic family 1(CFC1) |
| CFC1 | DQ786275 | Homo sapiens | cripto, FRL-1, cryptic family 1(CFC1) |
| CFC1 | AF312769 | Homo sapiens | cripto, FRL-1, cryptic family 1(CFC1) |
| CFC1 | BM509218 | Homo sapiens | cripto, FRL-1, cryptic family 1(CFC1) |
| COL3A1 | X14420 | Homo sapiens | collagen type III alpha 1 chain(COL3A1) |
| COL3A1 | X15332 | Homo sapiens | collagen type III alpha 1 chain(COL3A1) |
| COL3A1 | S79877 | Homo sapiens | collagen type III alpha 1 chain(COL3A1) |
| COL3A1 | X07240 | Homo sapiens | collagen type III alpha 1 chain(COL3A1) |
| COL3A1 | M11134 | Homo sapiens | collagen type III alpha 1 chain(COL3A1) |
| COL3A1 | M13146 | Homo sapiens | collagen type III alpha 1 chain(COL3A1) |
| COL3A1 | M59227 | Homo sapiens | collagen type III alpha 1 chain(COL3A1) |
| COL3A1 | AI755052 | Homo sapiens | collagen type III alpha 1 chain(COL3A1) |
| COL3A1 | BC028178 | Homo sapiens | collagen type III alpha 1 chain(COL3A1) |
| COL3A1 | NM_000090 | Homo sapiens | collagen type III alpha 1 chain(COL3A1) |
| COL3A1 | BP374999 | Homo sapiens | collagen type III alpha 1 chain(COL3A1) |
| COL3A1 | X06700 | Homo sapiens | collagen type III alpha 1 chain(COL3A1) |
| COL3A1 | X01655 | Homo sapiens | collagen type III alpha 1 chain(COL3A1) |
| COL3A1 | X01742 | Homo sapiens | collagen type III alpha 1 chain(COL3A1) |
| COL3A1 | AK091853 | Homo sapiens | collagen type III alpha 1 chain(COL3A1) |
| COL3A1 | AK308010 | Homo sapiens | collagen type III alpha 1 chain(COL3A1) |
| KRT19 | BC084574 | Homo sapiens | keratin 19(KRT19) |
| KRT19 | BG750971 | Homo sapiens | keratin 19(KRT19) |
| KRT19 | AB041267 | Homo sapiens | keratin 19(KRT19) |
| KRT19 | Y00503 | Homo sapiens | keratin 19(KRT19) |
| KRT19 | BP328445 | Homo sapiens | keratin 19(KRT19) |
| KRT19 | BG490639 | Homo sapiens | keratin 19(KRT19) |
| KRT19 | AB041268 | Homo sapiens | keratin 19(KRT19) |
| KRT19 | BC067744 | Homo sapiens | keratin 19(KRT19) |
| KRT19 | BC010409 | Homo sapiens | keratin 19(KRT19) |
| KRT19 | AK313261 | Homo sapiens | keratin 19(KRT19) |
| KRT19 | BC007628 | Homo sapiens | keratin 19(KRT19) |
| KRT19 | NM_002276 | Homo sapiens | keratin 19(KRT19) |
| KRT19 | BC002539 | Homo sapiens | keratin 19(KRT19) |
| KRT19 | DA068330 | Homo sapiens | keratin 19(KRT19) |
| KRT19 | DQ895373 | Homo sapiens | keratin 19(KRT19) |
| KRT19 | DQ892179 | Homo sapiens | keratin 19(KRT19) |
| DES | NM_001927 | Homo sapiens | desmin(DES) |
| DES | AK098332 | Homo sapiens | desmin(DES) |
| DES | U59167 | Homo sapiens | desmin(DES) |
| DES | AK022087 | Homo sapiens | desmin(DES) |
| DES | JX114780 | Homo sapiens | desmin(DES) |
| DES | AF055083 | Homo sapiens | desmin(DES) |
| DES | DQ104336 | Homo sapiens | desmin(DES) |
| DES | AK300654 | Homo sapiens | desmin(DES) |
| DES | DQ104337 | Homo sapiens | desmin(DES) |
| DES | EF617312 | Homo sapiens | desmin(DES) |
| DES | DQ104335 | Homo sapiens | desmin(DES) |
| DES | BQ941246 | Homo sapiens | desmin(DES) |
| DES | DQ895482 | Homo sapiens | desmin(DES) |
| DES | AF137053 | Homo sapiens | desmin(DES) |
| DES | AF167579 | Homo sapiens | desmin(DES) |
| DES | AY125465 | Homo sapiens | desmin(DES) |
| DES | AF487828 | Homo sapiens | desmin(DES) |
| DES | BC010072 | Homo sapiens | desmin(DES) |
| DES | AF521879 | Homo sapiens | desmin(DES) |
| DES | AF527578 | Homo sapiens | desmin(DES) |
| DES | AJ132926 | Homo sapiens | desmin(DES) |
| DES | AY083345 | Homo sapiens | desmin(DES) |
| DES | AF055081 | Homo sapiens | desmin(DES) |
| DES | AF055082 | Homo sapiens | desmin(DES) |
| DES | AK097038 | Homo sapiens | desmin(DES) |
| DES | BC032116 | Homo sapiens | desmin(DES) |
| DES | AF486807 | Homo sapiens | desmin(DES) |
| DES | DQ892282 | Homo sapiens | desmin(DES) |
| DES | AK123787 | Homo sapiens | desmin(DES) |
| DES | AL541778 | Homo sapiens | desmin(DES) |
| TTC6 | BC103914 | Homo sapiens | tetratricopeptide repeat domain 6(TTC6) |
| TTC6 | BC103915 | Homo sapiens | tetratricopeptide repeat domain 6(TTC6) |
| TTC6 | BC103916 | Homo sapiens | tetratricopeptide repeat domain 6(TTC6) |
| TTC6 | XR_001750287 | Homo sapiens | tetratricopeptide repeat domain 6(TTC6) |
| TTC6 | XM_011537430 | Homo sapiens | 6(TTC6) tetratricopeptide repeat domain |
| TTC6 | NM_001310135 | Homo sapiens | tetratricopeptide repeat domain 6(TTC6) |
| TTC6 | XM_011537431 | Homo sapiens | tetratricopeptide repeat domain 6(TTC6) |
| TTC6 | XM_017021254 | Homo sapiens | tetratricopeptide repeat domain 6(TTC6) |
| TTC6 | XM_017021255 | Homo sapiens | tetratricopeptide repeat domain 6(TTC6) |
| TTC6 | XR_943762 | Homo sapiens | tetratricopeptide repeat domain 6(TTC6) |
| TTC6 | XM_017021256 | Homo sapiens | tetratricopeptide repeat domain 6(TTC6) |
| TTC6 | XM_017021257 | Homo sapiens | tetratricopeptide repeat domain 6(TTC6) |
| TTC6 | XM_011537432 | Homo sapiens | tetratricopeptide repeat domain 6(TTC6) |
| TTC6 | BC038110 | Homo sapiens | tetratricopeptide repeat domain 6(TTC6) |
| TTC6 | BC014342 | Homo sapiens | tetratricopeptide repeat domain 6(TTC6) |
| TTC6 | BX161415 | Homo sapiens | tetratricopeptide repeat domain 6(TTC6) |
| TTC6 | NM_001007795 | Homo sapiens | tetratricopeptide repeat domain 6(TTC6) |
| PPP2R2B | DQ891785 | Homo sapiens | protein phosphatase 2 regulatory subunit Bbeta(PPP2R2B) |
| PPP2R2B | NM_001271899 | Homo sapiens | protein phosphatase 2 regulatory subunit Bbeta(PPP2R2B) |
| PPP2R2B | BI669304 | Homo sapiens | protein phosphatase 2 regulatory subunit Bbeta(PPP2R2B) |
| PPP2R2B | BP195204 | Homo sapiens | protein phosphatase 2 regulatory subunit Bbeta(PPP2R2B) |
| PPP2R2B | AK056192 | Homo sapiens | protein phosphatase 2 regulatory subunit Bbeta(PPP2R2B) |
| PPP2R2B | AK289717 | Homo sapiens | protein phosphatase 2 regulatory subunit Bbeta(PPP2R2B) |
| PPP2R2B | NM_001271948 | Homo sapiens | protein phosphatase 2 regulatory subunit Bbeta(PPP2R2B) |
| PPP2R2B | AK314810 | Homo sapiens | protein phosphatase 2 regulatory subunit Bbeta(PPP2R2B) |
| PPP2R2B | AL540315 | Homo sapiens | protein phosphatase 2 regulatory subunit Bbeta(PPP2R2B) |
| PPP2R2B | BC031790 | Homo sapiens | protein phosphatase 2 regulatory subunit Bbeta(PPP2R2B) |
| PPP2R2B | DA158596 | Homo sapiens | protein phosphatase 2 regulatory subunit Bbeta(PPP2R2B) |
| PPP2R2B | M64930 | Homo sapiens | protein phosphatase 2 regulatory subunit Bbeta(PPP2R2B) |
| PPP2R2B | DA155307 | Homo sapiens | protein phosphatase 2 regulatory subunit Bbeta(PPP2R2B) |
| PPP2R2B | NM_181677 | Homo sapiens | protein phosphatase 2 regulatory subunit Bbeta(PPP2R2B) |
| PPP2R2B | NM_181678 | Homo sapiens | protein phosphatase 2 regulatory subunit Bbeta(PPP2R2B) |
| PPP2R2B | DA365141 | Homo sapiens | protein phosphatase 2 regulatory subunit Bbeta(PPP2R2B) |
| PPP2R2B | NM_001271900 | Homo sapiens | protein phosphatase 2 regulatory subunit Bbeta(PPP2R2B) |
| PPP2R2B | NM_181675 | Homo sapiens | protein phosphatase 2 regulatory subunit Bbeta(PPP2R2B) |
| PPP2R2B | DQ894969 | Homo sapiens | protein phosphatase 2 regulatory subunit Bbeta(PPP2R2B) |
| PPP2R2B | NM_181676 | Homo sapiens | protein phosphatase 2 regulatory subunit Bbeta(PPP2R2B) |
| PPP2R2B | BX464346 | Homo sapiens | protein phosphatase 2 regulatory subunit Bbeta(PPP2R2B) |
| PPP2R2B | NM_181674 | Homo sapiens | protein phosphatase 2 regulatory subunit Bbeta(PPP2R2B) |
| PPP2R2B | DA522334 | Homo sapiens | protein phosphatase 2 regulatory subunit Bbeta(PPP2R2B) |
| PPP2R2B | AI621232 | Homo sapiens | protein phosphatase 2 regulatory subunit Bbeta(PPP2R2B) |
| PPP2R2B | DA523686 | Homo sapiens | protein phosphatase 2 regulatory subunit Bbeta(PPP2R2B) |
| PPP2R2B | NR_073527 | Homo sapiens | protein phosphatase 2 regulatory subunit Bbeta(PPP2R2B) |
| PPP2R2B | NR_073526 | Homo sapiens | protein phosphatase 2 regulatory subunit Bbeta(PPP2R2B) |
| PPP2R2B | AK294659 | Homo sapiens | protein phosphatase 2 regulatory subunit Bbeta(PPP2R2B) |
| PPP2R2B | BX647887 | Homo sapiens | protein phosphatase 2 regulatory subunit Bbeta(PPP2R2B) |
| PPP2R2B | AK295347 | Homo sapiens | protein phosphatase 2 regulatory subunit Bbeta(PPP2R2B) |
| PPP2R2B | AV726282 | Homo sapiens | protein phosphatase 2 regulatory subunit Bbeta(PPP2R2B) |
| PPP2R2B | DA533318 | Homo sapiens | protein phosphatase 2 regulatory subunit Bbeta(PPP2R2B) |
| PMP22 | CD519144 | Homo sapiens | peripheral myelin protein 22(PMP22) |
| PMP22 | BX464118 | Homo sapiens | peripheral myelin protein 22(PMP22) |
| PMP22 | DB466564 | Homo sapiens | peripheral myelin protein 22(PMP22) |
| PMP22 | AK300690 | Homo sapiens | peripheral myelin protein 22(PMP22) |
| PMP22 | D11428 | Homo sapiens | peripheral myelin protein 22(PMP22) |
| PMP22 | CR541931 | Homo sapiens | peripheral myelin protein 22(PMP22) |
| PMP22 | CR541953 | Homo sapiens | peripheral myelin protein 22(PMP22) |
| PMP22 | KR259964 | Homo sapiens | peripheral myelin protein 22(PMP22) |
| PMP22 | KR259963 | Homo sapiens | peripheral myelin protein 22(PMP22) |
| PMP22 | KR259962 | Homo sapiens | peripheral myelin protein 22(PMP22) |
| PMP22 | BQ232542 | Homo sapiens | peripheral myelin protein 22(PMP22) |
| PMP22 | BC019040 | Homo sapiens | peripheral myelin protein 22(PMP22) |
| PMP22 | BQ694106 | Homo sapiens | peripheral myelin protein 22(PMP22) |
| PMP22 | HY099893 | Homo sapiens | peripheral myelin protein 22(PMP22) |
| PMP22 | AK290640 | Homo sapiens | peripheral myelin protein 22(PMP22) |
| PMP22 | NR_104017 | Homo sapiens | peripheral myelin protein 22(PMP22) |
| PMP22 | NR_104018 | Homo sapiens | peripheral myelin protein 22(PMP22) |
| PMP22 | DQ895863 | Homo sapiens | peripheral myelin protein 22(PMP22) |
| PMP22 | BG424796 | Homo sapiens | peripheral myelin protein 22(PMP22) |
| PMP22 | CK818720 | Homo sapiens | peripheral myelin protein 22(PMP22) |
| PMP22 | DB504971 | Homo sapiens | peripheral myelin protein 22(PMP22) |
| PMP22 | NM_000304 | Homo sapiens | peripheral myelin protein 22(PMP22) |
| PMP22 | NM_153322 | Homo sapiens | peripheral myelin protein 22(PMP22) |
| PMP22 | DQ892623 | Homo sapiens | peripheral myelin protein 22(PMP22) |
| PMP22 | NM_001330143 | Homo sapiens | peripheral myelin protein 22(PMP22) |
| PMP22 | NM_153321 | Homo sapiens | peripheral myelin protein 22(PMP22) |
| PMP22 | L03203 | Homo sapiens | peripheral myelin protein 22(PMP22) |
| PMP22 | M94048 | Homo sapiens | peripheral myelin protein 22(PMP22) |
| PMP22 | DV460945 | Homo sapiens | peripheral myelin protein 22(PMP22) |
| PMP22 | XM_017024775 | Homo sapiens | peripheral myelin protein 22(PMP22) |
| PMP22 | HY119295 | Homo sapiens | peripheral myelin protein 22(PMP22) |
| PMP22 | NM_001281455 | Homo sapiens | peripheral myelin protein 22(PMP22) |
| PMP22 | XM_017024776 | Homo sapiens | peripheral myelin protein 22(PMP22) |
| PMP22 | BF725606 | Homo sapiens | peripheral myelin protein 22(PMP22) |
| PMP22 | NM_001281456 | Homo sapiens | peripheral myelin protein 22(PMP22) |
| PMP22 | X65968 | Homo sapiens | peripheral myelin protein 22(PMP22) |
| PMP22 | BQ212717 | Homo sapiens | peripheral myelin protein 22(PMP22) |
| PMP22 | BC091499 | Homo sapiens | peripheral myelin protein 22(PMP22) |
| PMP22 | AL833462 | Homo sapiens | peripheral myelin protein 22(PMP22) |
| PLP1 | M27110 | Homo sapiens | proteolipid protein 1(PLP1) |
| PLP1 | AK295374 | Homo sapiens | proteolipid protein 1(PLP1) |
| PLP1 | NM_000533 | Homo sapiens | proteolipid protein 1(PLP1) |
| PLP1 | DC342996 | Homo sapiens | proteolipid protein 1(PLP1) |
| PLP1 | BQ723748 | Homo sapiens | proteolipid protein 1(PLP1) |
| PLP1 | CR536542 | Homo sapiens | proteolipid protein 1(PLP1) |
| PLP1 | AK292728 | Homo sapiens | proteolipid protein 1(PLP1) |
| PLP1 | BX445448 | Homo sapiens | proteolipid protein 1(PLP1) |
| PLP1 | AK128782 | Homo sapiens | proteolipid protein 1(PLP1) |
| PLP1 | AV731932 | Homo sapiens | proteolipid protein 1(PLP1) |
| PLP1 | AI205189 | Homo sapiens | proteolipid protein 1(PLP1) |
| PLP1 | AK295388 | Homo sapiens | proteolipid protein 1(PLP1) |
| PLP1 | AK309239 | Homo sapiens | proteolipid protein 1(PLP1) |
| PLP1 | NM_001305004 | Homo sapiens | proteolipid protein 1(PLP1) |
| PLP1 | BQ717999 | Homo sapiens | proteolipid protein 1(PLP1) |
| PLP1 | M17085 | Homo sapiens | proteolipid protein 1(PLP1) |
| PLP1 | AK308966 | Homo sapiens | proteolipid protein 1(PLP1) |
| PLP1 | KU178261 | Homo sapiens | proteolipid protein 1(PLP1) |
| PLP1 | KU178262 | Homo sapiens | proteolipid protein 1(PLP1) |
| PLP1 | DC342197 | Homo sapiens | proteolipid protein 1(PLP1) |
| PLP1 | NM_001128834 | Homo sapiens | proteolipid protein 1(PLP1) |
| PLP1 | AK312340 | Homo sapiens | proteolipid protein 1(PLP1) |
| PLP1 | BC095452 | Homo sapiens | proteolipid protein 1(PLP1) |
| PLP1 | DA299940 | Homo sapiens | proteolipid protein 1(PLP1) |
| PLP1 | EB387051 | Homo sapiens | proteolipid protein 1(PLP1) |
| PLP1 | BC002665 | Homo sapiens | proteolipid protein 1(PLP1) |
| PLP1 | M54927 | Homo sapiens | proteolipid protein 1(PLP1) |
| PLP1 | CA389667 | Homo sapiens | proteolipid protein 1(PLP1) |
| PLP1 | NM_199478 | Homo sapiens | proteolipid protein 1(PLP1) |
| PLP1 | BT019602 | Homo sapiens | proteolipid protein 1(PLP1) |
| PLP1 | BT019601 | Homo sapiens | proteolipid protein 1(PLP1) |
| RMST | AF429306 | Homo sapiens | rhabdomyosarcoma 2 associated transcript(non-proteincoding)(RMST) |
| RMST | AK056164 | Homo sapiens | rhabdomyosarcoma 2 associated transcript(non-protein coding)(RMST) |
| RMST | AF429305 | Homo sapiens | rhabdomyosarcoma 2 associated transcript(non-protein coding)(RMST) |
| RMST | NR_024037 | Homo sapiens | rhabdomyosarcoma 2 associated transcript(non-protein coding)(RMST) |
| SPC24 | BQ216591 | Homo sapiens | SPC24, NDC80 kinetochore complex component(SPC24) |
| SPC24 | XM_005259753 | Homo sapiens | SPC24, NDC80 kinetochore complex component(SPC24) |
| SPC24 | AY456387 | Homo sapiens | SPC24, NDC80 kinetochore complex component(SPC24) |
| SPC24 | NM_182513 | Homo sapiens | SPC24, NDC80 kinetochore complex component(SPC24) |
| SPC24 | BQ061822 | Homo sapiens | SPC24, NDC80 kinetochore complex component(SPC24) |
| SPC24 | XM_011527702 | Homo sapiens | SPC24, NDC80 kinetochore complex component(SPC24) |
| SPC24 | AK303157 | Homo sapiens | SPC24, NDC80 kinetochore complex component(SPC24) |
| SPC24 | AK075287 | Homo sapiens | SPC24, NDC80 kinetochore complex component(SPC24) |
| SPC24 | AK303266 | Homo sapiens | SPC24, NDC80 kinetochore complex component(SPC24) |
| SPC24 | BC105039 | Homo sapiens | SPC24, NDC80 kinetochore complex component(SPC24) |
| SPC24 | HY082333 | Homo sapiens | SPC24, NDC80 kinetochore complex component(SPC24) |
| SPC24 | NM_001317033 | Homo sapiens | SPC24, NDC80 kinetochore complex component(SPC24) |
| SPC24 | BC105037 | Homo sapiens | SPC24, NDC80 kinetochore complex component(SPC24) |
| SPC24 | NM_001317032 | Homo sapiens | SPC24, NDC80 kinetochore complex component(SPC24) |
| SPC24 | NM_001317031 | Homo sapiens | SPC24, NDC80 kinetochore complex component(SPC24) |
| ADGRL3 | AB018311 | Homo sapiens | adhesion G protein-coupled receptor L3(ADGRL3) |
| ADGRL3 | XM_017007942 | Homo sapiens | adhesion G protein-coupled receptor L3(ADGRL3) |
| ADGRL3 | XM_017007941 | Homo sapiens | adhesion G protein-coupled receptor L3(ADGRL3) |
| ADGRL3 | XM_017007940 | Homo sapiens | adhesion G protein-coupled receptor L3(ADGRL3) |
| ADGRL3 | XM_017007929 | Homo sapiens | adhesion G protein-coupled receptor L3(ADGRL3) |
| ADGRL3 | XM_011531791 | Homo sapiens | adhesion G protein-coupled receptor L3(ADGRL3) |
| ADGRL3 | AK294689 | Homo sapiens | adhesion G protein-coupled receptor L3(ADGRL3) |
| ADGRL3 | AK309793 | Homo sapiens | adhesion G protein-coupled receptor L3(ADGRL3) |
| ADGRL3 | AK299651 | Homo sapiens | adhesion G protein-coupled receptor L3(ADGRL3) |
| ADGRL3 | BC039452 | Homo sapiens | adhesion G protein-coupled receptor L3(ADGRL3) |
| ADGRL3 | DA808156 | Homo sapiens | adhesion G protein-coupled receptor L3(ADGRL3) |
| ADGRL3 | XM_017007934 | Homo sapiens | adhesion G protein-coupled receptor L3(ADGRL3) |
| ADGRL3 | AK000781 | Homo sapiens | adhesion G protein-coupled receptor L3(ADGRL3) |
| ADGRL3 | XM_017007933 | Homo sapiens | adhesion G protein-coupled receptor L3(ADGRL3) |
| ADGRL3 | XM_017007932 | Homo sapiens | adhesion G protein-coupled receptor L3(ADGRL3) |
| ADGRL3 | XM_017007931 | Homo sapiens | adhesion G protein-coupled receptor L3(ADGRL3) |
| ADGRL3 | XM_017007930 | Homo sapiens | adhesion G protein-coupled receptor L3(ADGRL3) |
| ADGRL3 | XM_017007939 | Homo sapiens | adhesion G protein-coupled receptor L3(ADGRL3) |
| ADGRL3 | XM_017007938 | Homo sapiens | adhesion G protein-coupled receptor L3(ADGRL3) |
| ADGRL3 | XM_017007937 | Homo sapiens | adhesion G protein-coupled receptor L3(ADGRL3) |
| ADGRL3 | XM_017007936 | Homo sapiens | adhesion G protein-coupled receptor L3(ADGRL3) |
| ADGRL3 | XM_017007935 | Homo sapiens | adhesion G protein-coupled receptor L3(ADGRL3) |
| ADGRL3 | NM_015236 | Homo sapiens | adhesion G protein-coupled receptor L3(ADGRL3) |
| ADGRL3 | NM_001322246 | Homo sapiens | adhesion G protein-coupled receptor L3(ADGRL3) |
| ADGRL3 | XM_011531788 | Homo sapiens | adhesion G protein-coupled receptor L3(ADGRL3) |
| ADGRL3 | NM_001322402 | Homo sapiens | adhesion G protein-coupled receptor L3(ADGRL3) |
| ADGRL3 | AK094703 | Homo sapiens | adhesion G protein-coupled receptor L3(ADGRL3) |

### REFERENCES

Alhawaj, A. F. (2021). Stem cell-based therapy for hirschsprung disease, do we have the guts to treat?Gene Ther.
Apsel, B., Blair, J. A., Gonzalez, B., Nazif, T. M., Feldman, M. E., Aizenstein, B., Hoffman, R., Williams, R. L., Shokat, K. M., and Knight, Z. A. (2008). Targeted polypharmacology: discovery of dual inhibitors of tyrosine and phosphoinositide kinases. Nat. Chem. Biol. 4, 691-699.
Baetge, G., and Gershon, M. D. (1989). Transient catecholaminergic (TC) cells in the vagus nerves and bowel of fetal mice: relationship to the development of enteric neurons. Dev. Biol. 132, 189-211.
Baetge, G., Pintar, J. E., and Gershon, M. D. (1990). Transiently catecholaminergic (TC) cells in the bowel of the fetal rat: precursors of noncatecholaminergic enteric neurons. Dev. Biol. 141, 353-380.
Barajas-López, C., and Huizinga, J. D. (1989). Different mechanisms of contraction generation in circular muscle of canine colon. Am. J. Physiol. 256, G570-580.
Barber, K., Studer, L., and Fattahi, F. (2019). Derivation of enteric neuron lineages from human pluripotent stem cells. Nat. Protoc. 14, 1261-1279.
Bergner, A. J., Stamp, L. A., Gonsalvez, D. G., Allison, M. B., Olson, D. P., Myers, M. G., Anderson, C. R., and Young, H. M. (2014). Birthdating of myenteric neuron subtypes in the small intestine of the mouse. J. Comp. Neurol. 522, 514-527.
Bódi, N., Szalai, Z., and Bagyánszki, M. (2019). Nitrergic Enteric Neurons in Health and Disease-Focus on Animal Models. Int. J. Mol. Sci. 20, E2003.
Bondurand, N., and Southard-Smith, E. M. (2016). Mouse models of Hirschsprung disease and other developmental disorders of the enteric nervous system: Old and new players. Dev. Biol. 417, 139-157.
Bredt, D. S., Hwang, P. M., and Snyder, S. H. (1990). Localization of nitric oxide synthase indicating a neural role for nitric oxide. Nature 347, 768-770.
Brehmer, A. (2021). Classification of human enteric neurons. Histochem. Cell Biol. 156, 95-108.
Bullitt, E. (1990). Expression of c-fos-like protein as a marker for neuronal activity following noxious stimulation in the rat. J. Comp. Neurol. 296, 517-530.
Bult, H., Boeckxstaens, G. E., Pelckmans, P. A., Jordaens, F. H., Van Maercke, Y. M., and Herman, A. G. (1990). Nitric oxide as an inhibitory non-adrenergic non-cholinergic neurotransmitter. Nature 345, 346-347.
Burns, A. J., and Douarin, N. M. (1998). The sacral neural crest contributes neurons and glia to the post-umbilical gut: spatiotemporal analysis of the development of the enteric nervous system. Dev. Camb. Engl. 125, 4335-4347.
Burns, A. J., Lomax, A. E., Torihashi, S., Sanders, K. M., and Ward, S. M. (1996). Interstitial cells of Cajal mediate inhibitory neurotransmission in the stomach. Proc. Natl. Acad. Sci. U.S.A 93, 12008-12013.
Burns, A. J., Goldstein, A. M., Newgreen, D. F., Stamp, L., Schäfer, K.-H., Metzger, M., Hotta, R., Young, H. M., Andrews, P. W., Thapar, N., et al. (2016). White paper on guidelines concerning enteric nervous system stem cell therapy for enteric neuropathies. Dev. Biol. 417, 229-251.
Camilleri, M. (2021). Gastrointestinal motility disorders in neurologic disease. J. Clin. Invest. 131, 143771.
Camilleri, M., Bharucha, A. E., and Farrugia, G. (2011). Epidemiology, mechanisms, and management of diabetic gastroparesis. Clin. Gastroenterol. Hepatol. Off Clin. Pract. J. Am. Gastroenterol. Assoc. 9, 5-12; quiz e7.
De Giorgio, R. (2006). The Enteric Nervous System, J. B. Furness. Blackwell Publishing, Oxford (2006), (274 pp., Price GBP 55.00, ISBN 1-4051-3376-7). Dig. Liver Dis. 38, 441.
De Giorgio, R., Bianco, F., Latorre, R., Caio, G., Clavenzani, P., and Bonora, E. (2016). Enteric neuropathies: Yesterday, Today and Tomorrow. Adv. Exp. Med. Biol. 891, 123-133.
Drokhlyansky, E., Smillie, C. S., Van Wittenberghe, N., Ericsson, M., Griffin, G. K., Eraslan, G., Dionne, D., Cuoco, M. S., Goder-Reiser, M. N., Sharova, T., et al. (2020). The Human and Mouse Enteric Nervous System at Single-Cell Resolution. Cell 182, 1606-1622.e23.
Fattahi, F., Steinbeck, J. A., Kriks, S., Tchieu, J., Zimmer, B., Kishinevsky, S., Zeltner, N., Mica, Y., El-Nachef, W., Zhao, H., et al. (2016). Deriving human ENS lineages for cell therapy and drug discovery in Hirschsprung disease. Nature 531, 105-109.
Fida, R., Lyster, D. J., Bywater, R. A., and Taylor, G. S. (1997). Colonic migrating motor complexes (CMMCs) in the isolated mouse colon. Neurogastroenterol. Motil. Off. J. Eur. Gastrointest. Motil. Soc. 9, 99-107.
Fung, C., and Vanden Berghe, P. (2020). Functional circuits and signal processing in the enteric nervous system. Cell. Mol. Life Sci. CMLS 77, 4505-4522.
Furness, J. B., Callaghan, B. P., Rivera, L. R., and Cho, H.-J. (2014). The enteric nervous system and gastrointestinal innervation: integrated local and central control. Adv. Exp. Med. Biol. 817, 39-71.
Gershon, M. D. (1999). The enteric nervous system: a second brain. Hosp. Pract. 1995 34, 31-32, 35-38, 41-42 passim.
Grubišić, V., and Gulbransen, B. D. (2017). Enteric glia: the most alimentary of all glia. J. Physiol. 595, 557-570.
Grundmann, D., Loris, E., Maas-Omlor, S., Huang, W., Scheller, A., Kirchhoff, F., and Schäfer, K.-H. (2019). Enteric Glia: S100, GFAP, and Beyond. Anat. Rec. Hoboken NJ 2007 302, 1333-1344.
Hamnett, R., Dershowitz, L. B., Sampathkumar, V., Wang, Z., Andrade, V. D., Kasthuri, N., Druckmann, S., and Kaltschmidt, J. A. (2021). Regional cytoarchitecture of the adult and developing mouse enteric nervous system.
Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29.
Heanue, T. A., and Pachnis, V. (2007). Enteric nervous system development and Hirschsprung's disease: advances in genetic and stem cell studies. Nat. Rev. Neurosci. 8, 466-479.
Hunt, S. P., Pini, A., and Evan, G. (1987). Induction of c-fos-like protein in spinal cord neurons following sensory stimulation. Nature 328, 632-634.
Lai, F. P.-L., Lau, S.-T., Wong, J. K.-L., Gui, H., Wang, R. X., Zhou, T., Lai, W. H., Tse, H.-F., Tam, P. K.-H., Garcia-Barcelo, M.-M., et al. (2017). Correction of Hirschsprung-Associated Mutations in Human Induced Pluripotent Stem Cells Via Clustered Regularly Interspaced Short Palindromic Repeats/Cas9, Restores Neural Crest Cell Function. Gastroenterology 153, 139153.e8.
Lake, J. I., and Heuckeroth, R. O. (2013). Enteric nervous system development: migration, differentiation, and disease. Am. J. Physiol.—Gastrointest. Liver Physiol. 305, G1-G24.
Long-Smith, C., O'Riordan, K. J., Clarke, G., Stanton, C., Dinan, T. G., and Cryan, J. F. (2020). Microbiota-Gut-Brain Axis: New Therapeutic Opportunities. Annu. Rev. Pharmacol. Toxicol. 60, 477-502.
Lyster, D. J., Bywater, R. A., and Taylor, G. S. (1995). Neurogenic control of myoelectric complexes in the mouse isolated colon. Gastroenterology 108, 1371-1378.
McCann, C. J., Cooper, J. E., Natarajan, D., Jevans, B., Burnett, L. E., Burns, A. J., and Thapar, N. (2017). Transplantation of enteric nervous system stem cells rescues nitric oxide synthase deficient mouse colon. Nat. Commun. 8, 15937.
Morarach, K., Mikhailova, A., Knoflach, V., Memic, F., Kumar, R., Li, W., Ernfors, P., and Marklund, U. (2021). Diversification of molecularly defined myenteric neuron classes revealed by single-cell RNA sequencing. Nat. Neurosci. 24, 34-46.
Muller, P. A., Koscsó, B., Rajani, G. M., Stevanovic, K., Berres, M.-L., Hashimoto, D., Mortha, A., Leboeuf, M., Li, X.-M., Mucida, D., et al. (2014). Crosstalk between muscularis macrophages and enteric neurons regulates gastrointestinal motility. Cell 158, 300-313.
Nagy, N., and Goldstein, A. M. (2017). Enteric nervous system development: A crest cell's journey from neural tube to colon. Semin. Cell Dev. Biol. 66, 94-106.
Niesler, B., Kuerten, S., Demir, I. E., and Schäfer, K.-H. (2021). Disorders of the enteric nervous system—a holistic view. Nat. Rev. Gastroenterol. Hepatol. 18, 393-410.
Obata, Y., and Pachnis, V. (2016). The Effect of Microbiota and the Immune System on the Development and Organization of the Enteric Nervous System. Gastroenterology 151, 836-844.
Obermayr, F., Stamp, L. A., Anderson, C. R., and Young, H. M. (2013). Genetic fate-mapping of tyrosine hydroxylase-expressing cells in the enteric nervous system. Neurogastroenterol. Motil. Off. J. Eur. Gastrointest. Motil. Soc. 25, e283-291.
Paridaen, J. T. M. L., and Huttner, W. B. (2014). Neurogenesis during development of the vertebrate central nervous system. EMBO Rep. 15, 351-364.
Pesce, M., Borrelli, O., Saliakellis, E., and Thapar, N. (2018). Gastrointestinal Neuropathies: New Insights and Emerging Therapies. Gastroenterol. Clin. North Am. 47, 877-894.
Qu, Z.-D., Thacker, M., Castelucci, P., Bagyánszki, M., Epstein, M. L., and Furness, J. B. (2008). Immunohistochemical analysis of neuron types in the mouse small intestine. Cell Tissue Res. 334, 147-161.
Rao, M., and Gershon, M. D. (2018). Enteric nervous system development: what could possibly go wrong?Nat. Rev. Neurosci. 19, 552-565.
Rivera, L. R., Poole, D. P., Thacker, M., and Furness, J. B. (2011). The involvement of nitric oxide synthase neurons in enteric neuropathies. Neurogastroenterol. Motil. Off J. Eur. Gastrointest. Motil. Soc. 23, 980-988.
Rothman, T. P., Tennyson, V. M., and Gershon, M. D. (1986). Colonization of the bowel by the precursors of enteric glia: studies of normal and congenitally aganglionic mutant mice. J. Comp. Neurol. 252, 493-506.
Rowitch, D. H., and Kriegstein, A. R. (2010). Developmental genetics of vertebrate glial-cell specification. Nature 468, 214-222.
Santos, P. L., Brito, R. G., Matos, J. P. S. C. F., Quintans, J. S. S., and Quintans-Júnior, L. J. (2018). Fos Protein as a Marker of Neuronal Activity: a Useful Tool in the Study of the Mechanism of Action of Natural Products with Analgesic Activity. Mol. Neurobiol. 55, 4560-4579.
Sasselli, V., Pachnis, V., and Burns, A. J. (2012). The enteric nervous system. Dev. Biol. 366, 64-73.
Schneider, S., Wright, C. M., and Heuckeroth, R. O. (2019). Unexpected Roles for the Second Brain: Enteric Nervous System as Master Regulator of Bowel Function. Annu. Rev. Physiol. 81, 235-259.
Serbedzija, G. N., Burgan, S., Fraser, SE., and Bronner-Fraser, M. (1991). Vital dye labelling demonstrates a sacral neural crest contribution to the enteric nervous system of chick and mouse embryos. Dev. Camb. Engl. 111, 857-866.
Smith, T. K., Reed, J. B., and Sanders, K. M. (1987). Origin and propagation of electrical slow waves in circular muscle of canine proximal colon. Am. J. Physiol. 252, C215-224.
Spear, E. T., Holt, E. A., Joyce, E. J., Haag, M. M., Mawe, S. M., Hennig, G. W., Lavoie, B., Applebee, A. M., Teuscher, C., and Mawe, G. M. (2018). Altered gastrointestinal motility involving autoantibodies in the experimental autoimmune encephalomyelitis model of multiple sclerosis. Neurogastroenterol. Motil. Off. J. Eur. Gastrointest. Motil. Soc. 30, e13349.
Steinbeck, J. A., Jaiswal, M. K., Calder, E. L., Kishinevsky, S., Weishaupt, A., Toyka, K. V., Goldstein, P. A., and Studer, L. (2016). Functional Connectivity under Optogenetic Control Allows Modeling of Human Neuromuscular Disease. Cell Stem Cell 18, 134-143.
Swaminathan, M., Hill-Yardin, E., Ellis, M., Zygorodimos, M., Johnston, L. A., Gwynne, R. M., and Bornstein, J. C. (2016). Video Imaging and Spatiotemporal Maps to Analyze Gastrointestinal Motility in Mice. J. Vis. Exp. JoVE 53828.
Ward, S. M., Xue, C., Shuttleworth, C. W., Bredt, D. S., Snyder, S. H., and Sanders, K. M. (1992). NADPH diaphorase and nitric oxide synthase colocalization in enteric neurons of canine proximal colon. Am. J. Physiol. 263, G277-284.
Wu, Y., Tamayo, P., and Zhang, K. (2018). Visualizing and Interpreting Single-Cell Gene Expression Datasets with Similarity Weighted Nonnegative Embedding. Cell Syst. 7, 656-666.e4.
Yarandi, S. S., and Srinivasan, S. (2014). Diabetic gastrointestinal motility disorders and the role of enteric nervous system: current status and future directions. Neurogastroenterol. Motil. Off. J. Eur. Gastrointest. Motil. Soc. 26, 611-624.
Yoo, B. B., and Mazmanian, S. K. (2017). The Enteric Network: Interactions between the Immune and Nervous Systems of the Gut. Immunity 46, 910-926.
Young, H. M., Furness, J. B., Shuttleworth, C. W., Bredt, D. S., and Snyder, S. H. (1992). Co-localization of nitric oxide synthase immunoreactivity and NADPH diaphorase staining in neurons of the guinea-pig intestine. Histochemistry 97, 375-378.
Young, H. M., Bergner, A. J., and Müller, T. (2003). Acquisition of neuronal and glial markers by neural crest-derived cells in the mouse intestine. J. Comp. Neurol. 456, 1-11.

1.-87. (canceled)

88. A composition comprising one or a plurality of enteric neurons, wherein the enteric neurons comprise SOX10 and at least one of: CD24, CD45RA, CD57, CD63, CD71, CD121b, CD147, CD164, CD184, CD193, CD243, CD275.

89. The composition of claim 88, wherein the composition comprises a plurality of enteric neurons arranged in a spheroid or gangloid.

90. A composition comprising one or a plurality of enteric glial cells, wherein the enteric glial cells comprise SOX10 and PMP22.

91. The composition of claim 90, wherein the one or plurality of enteric glial cells further comprise SB100.

92. The composition of claim 90, wherein the one or plurality of enteric glial cells further comprise PLP1.

93. The composition of claim 90, wherein the one or plurality of enteric glial cells further comprise AQP4.

94. The composition of claim 90, wherein the one or plurality of enteric glial cells further comprise GFAP.

95. The composition of claim 90, wherein the one or plurality of enteric glial cells further comprise MPZ.

96. The composition of claim 90, wherein the one or plurality of enteric glial cells further comprise MBP.

97. The composition of claim 90, wherein the one or plurality of enteric glial cells are from an induced pluripotent stem cell.

98. The composition of claim 90 further comprising one or a plurality of enteric neurons, wherein the enteric neurons comprise SOX10 and at least one of: CD24, CD45RA, CD57, CD63, CD71, CD121b, CD147, CD164, CD184, CD193, CD243, CD275.

99. The composition of claim 89 further comprising:
(i) one or a plurality of mesenchymal cells; and
(ii) one or a plurality of epithelial cells.

100. The composition of claim 99 further comprising:
(i) one or a plurality of enteric glial cells of claim 90; and/or
(ii) one or a plurality of smooth muscle cells.

101. A subject comprising one or a plurality of cells of claim 90.

102. A method of differentiating a neuronal cell to an enteric neuronal cell, the method comprising exposing an effective amount of a platelet-derived growth factor receptor (PDGFR) inhibitor or a pharmaceutically acceptable salt thereof to a neuronal cell for a time period sufficient to differentiate the neuronal cell to an enteric neuronal cell.

103. The method of claim 102, wherein the PDGFR inhibitor is selected from (Z)-orantinib, AC710, AC710 mesylate, AG 1295, amuvatinib, amuvatinib hydrochloride, avapritinib, axitinib, AZD2932, cediranib, cediranib maleate, chiauranib, CHIR-124, CP-673451, crenolanib, dovitinib, dovitinib lactate, dovitinib lactate, hydrate, dovitinib-D8, ENMD-2076, ENMD-2076 tartrate, flumatinib, flumatinib mesylate, GZD856, GZD856 formic, HG-7-85-01, hypothemycrin, ilorasertib, ilorasertib hydrochloride, imatinib, imatinib D4, imatinib D8, imatinib mesylate, JI-101, JNJ-10198409, KG5, Ki20227, lenvatinib, lenvatinib mesylate, linifanib, masitinib, masitinib mesylate, methylnissolin, multi-kinase inhibitor 1, N-(p-coumaroyl) serotonin, nintedanib, nintedanib esylate, NVP-ACC789, orantinib, pazopanib, pazopanib hydrochloride, PD-089828, PD-161570, PDGFRα kinase inhibitor 1, ponatinib, ponatinib D8, PP121, PP58, regorafenib, regorafenib D3, regorafenib hydrochloride, regorafenib monohydrate, ripretinib, sennoside B, seralutinib, SU5402, SU14813, SU14813 maleate, SU16f, SU4312, SU4984, sunitinib, sunitinib D10, sunitinib malate, sunitinib-d4, TAK-593, tandutinib, tandutinib hydrochloride, telatinib, telatinib mesylate, TG 100572, TG 100572 hydrochloride, TG 100801, TG 100801 hydrochloride, toceranib, toceranib phosphate, toceranib-d8, trapidil, tyrosine kinase-IN-1, tyrphostin AG1296, tryphostin AG1433, and vorolanib.

104. The method of claim 102, wherein the PDGFR inhibitor is selected from: 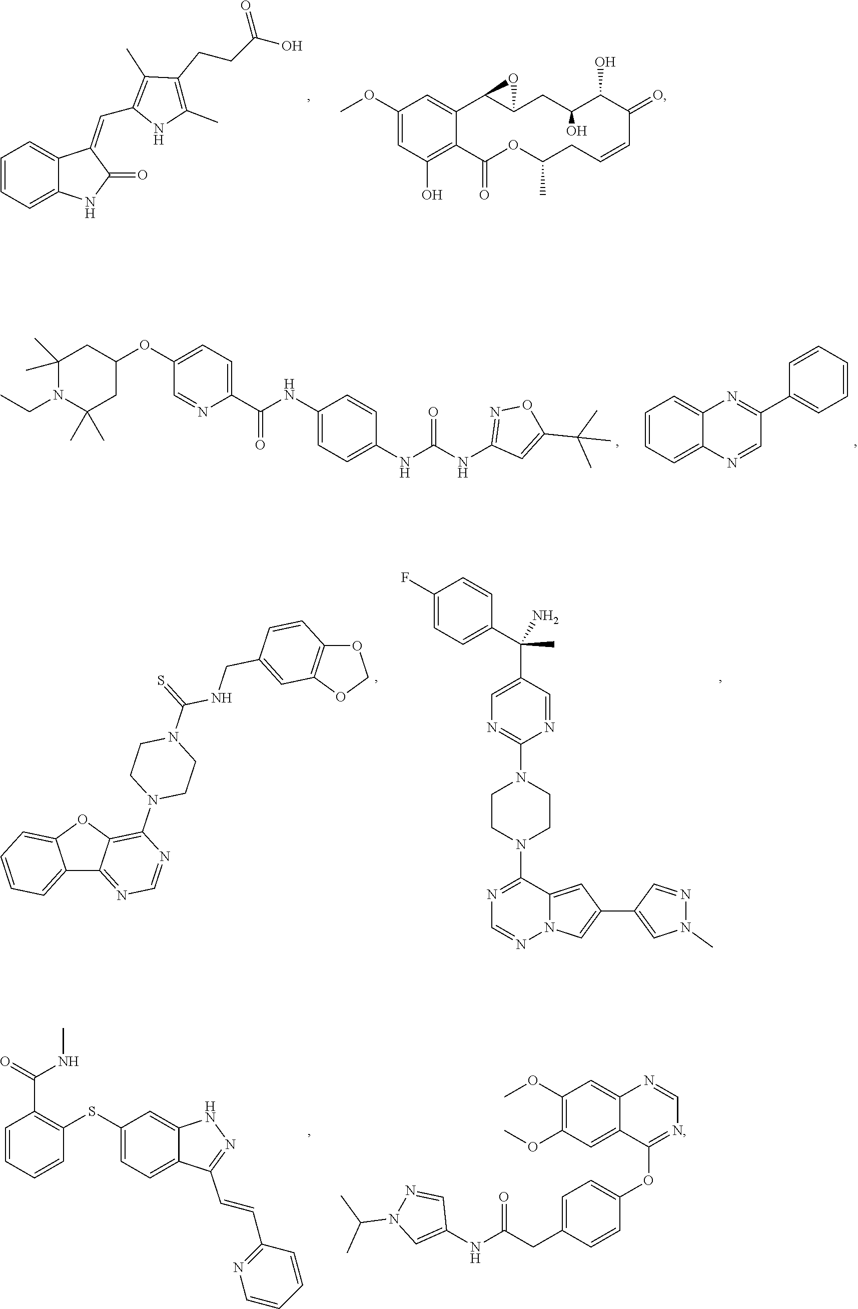 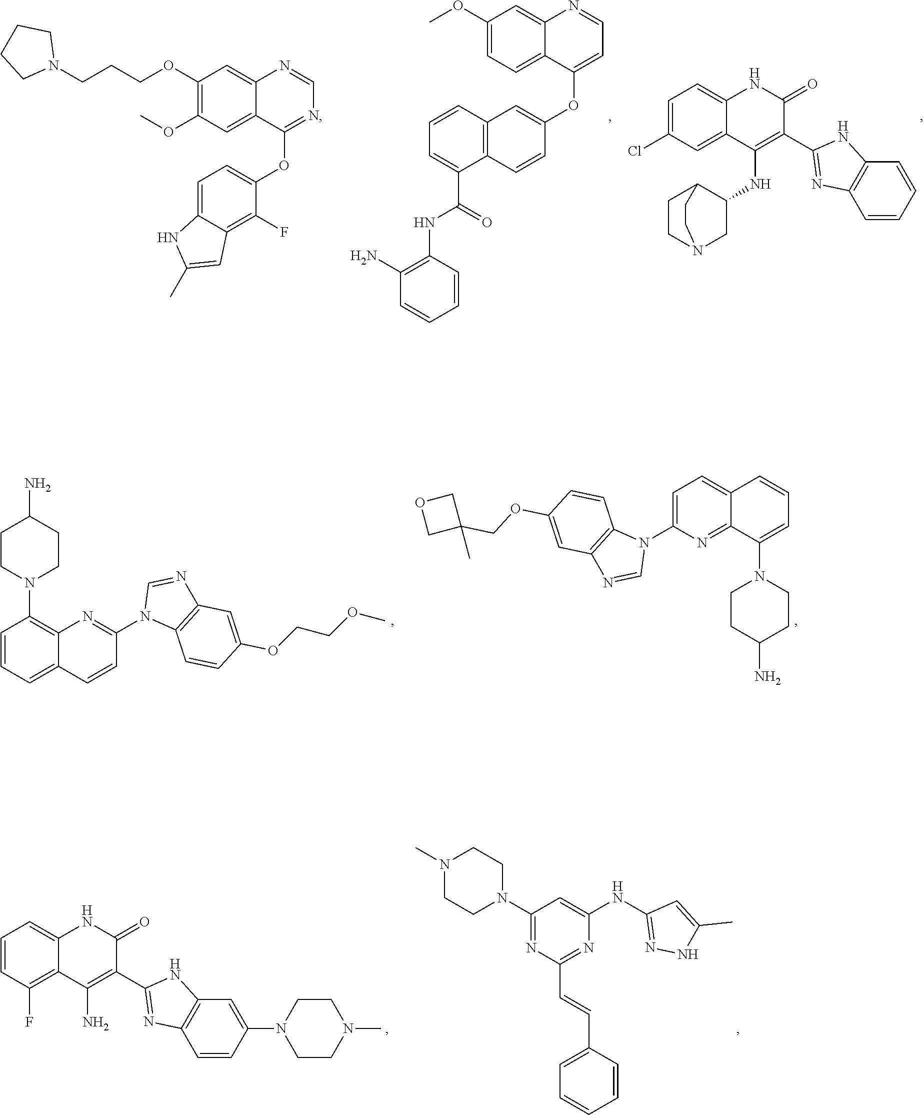 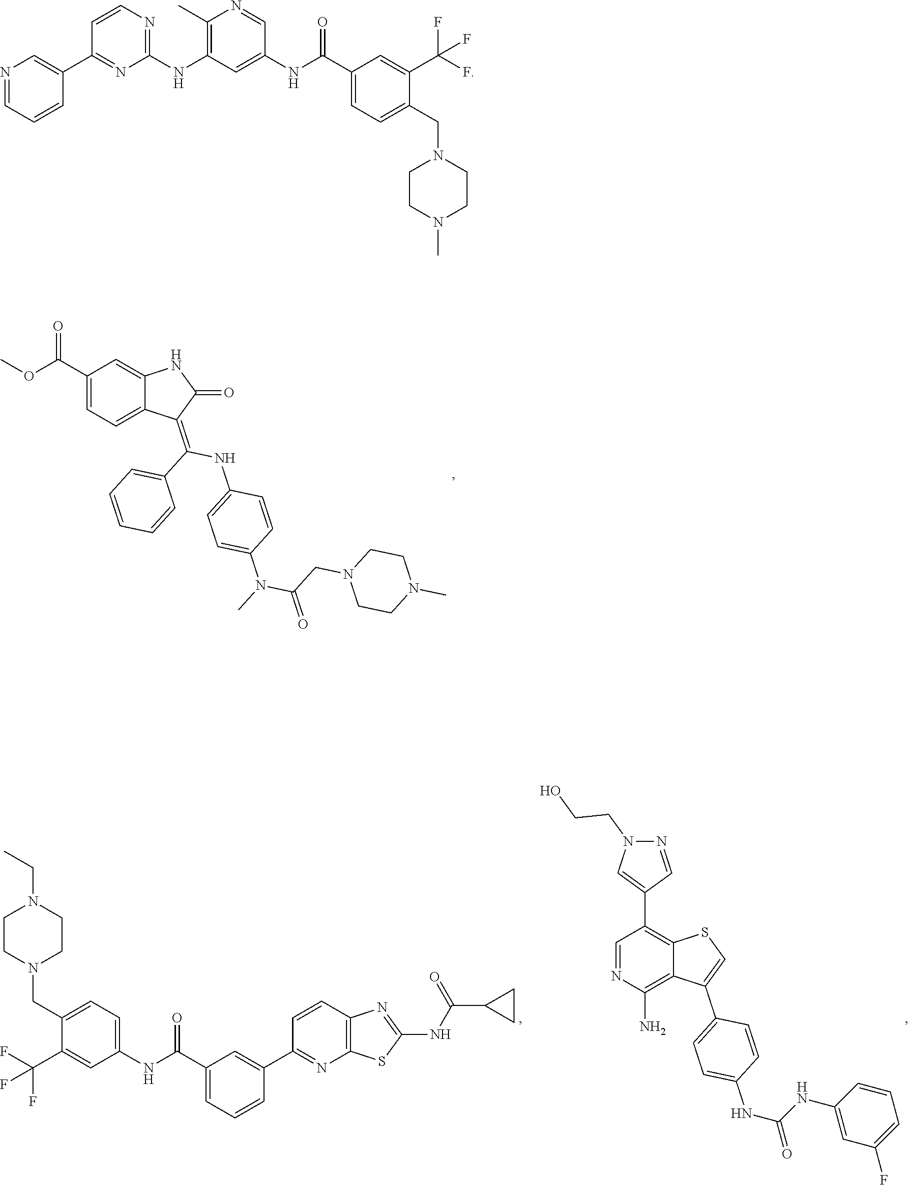 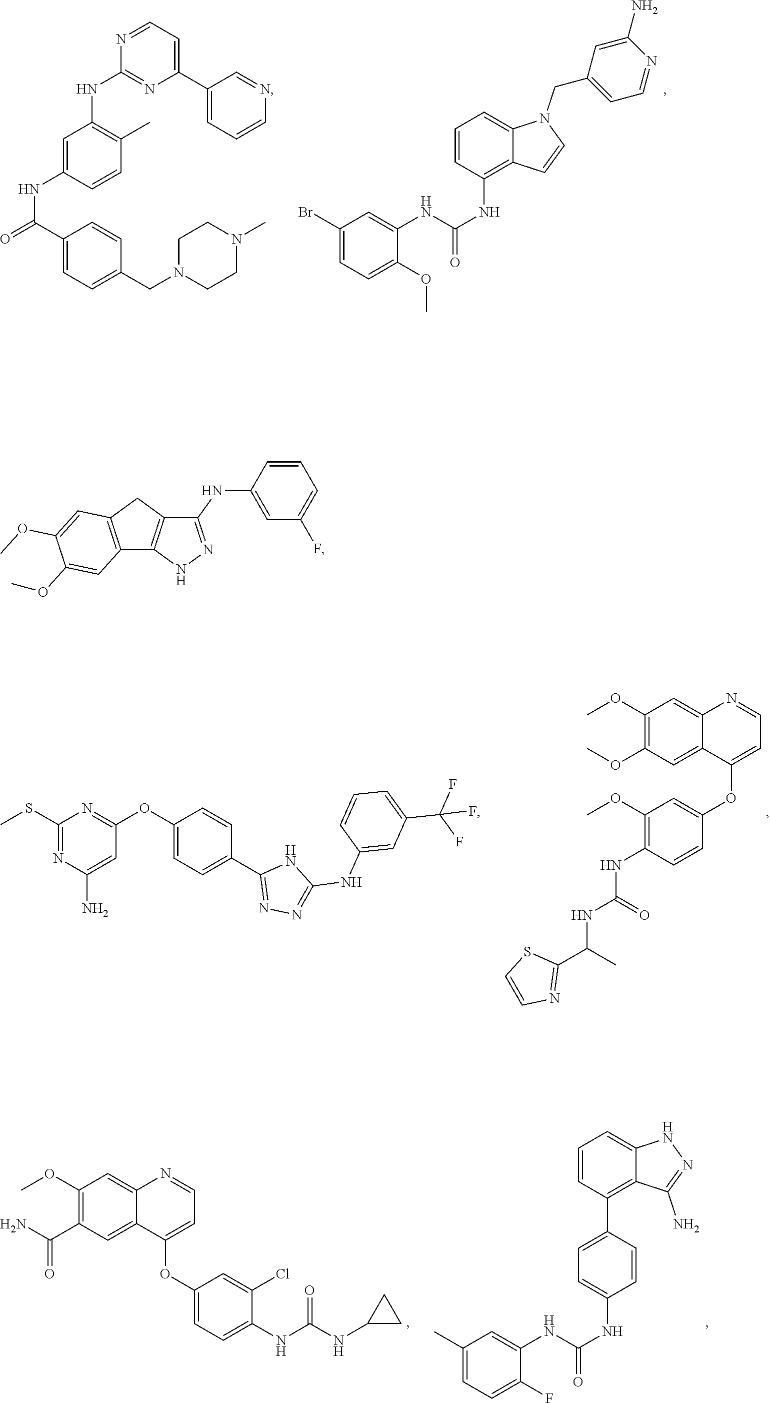 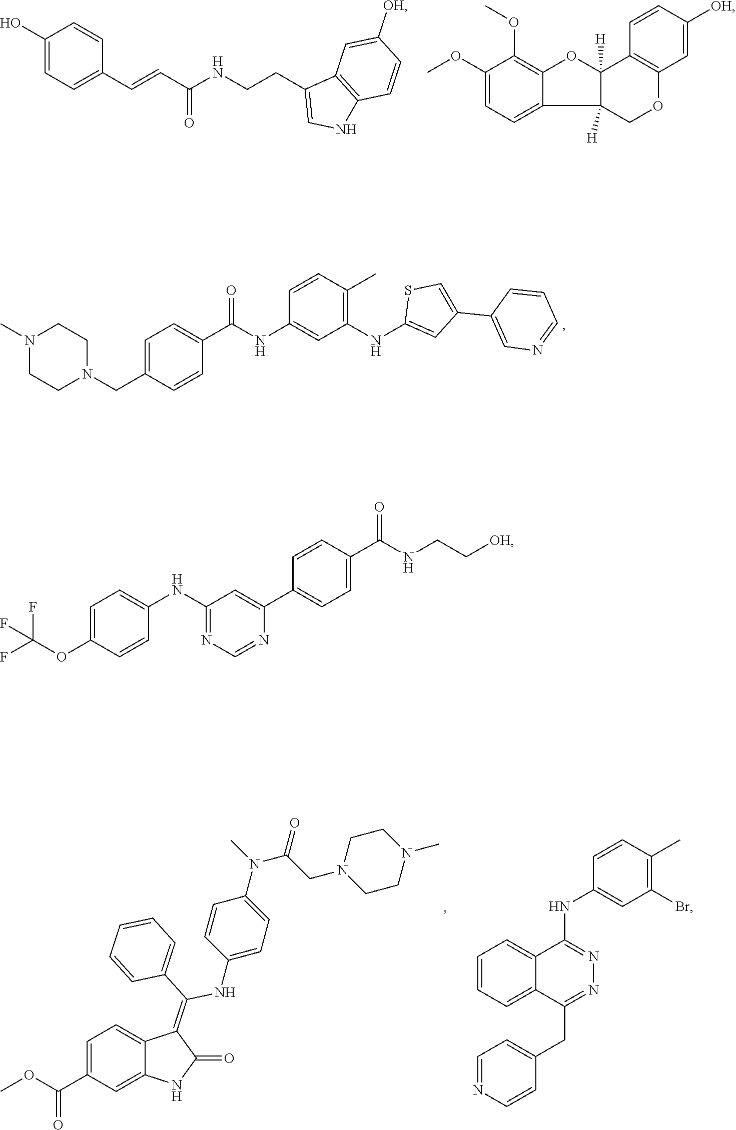 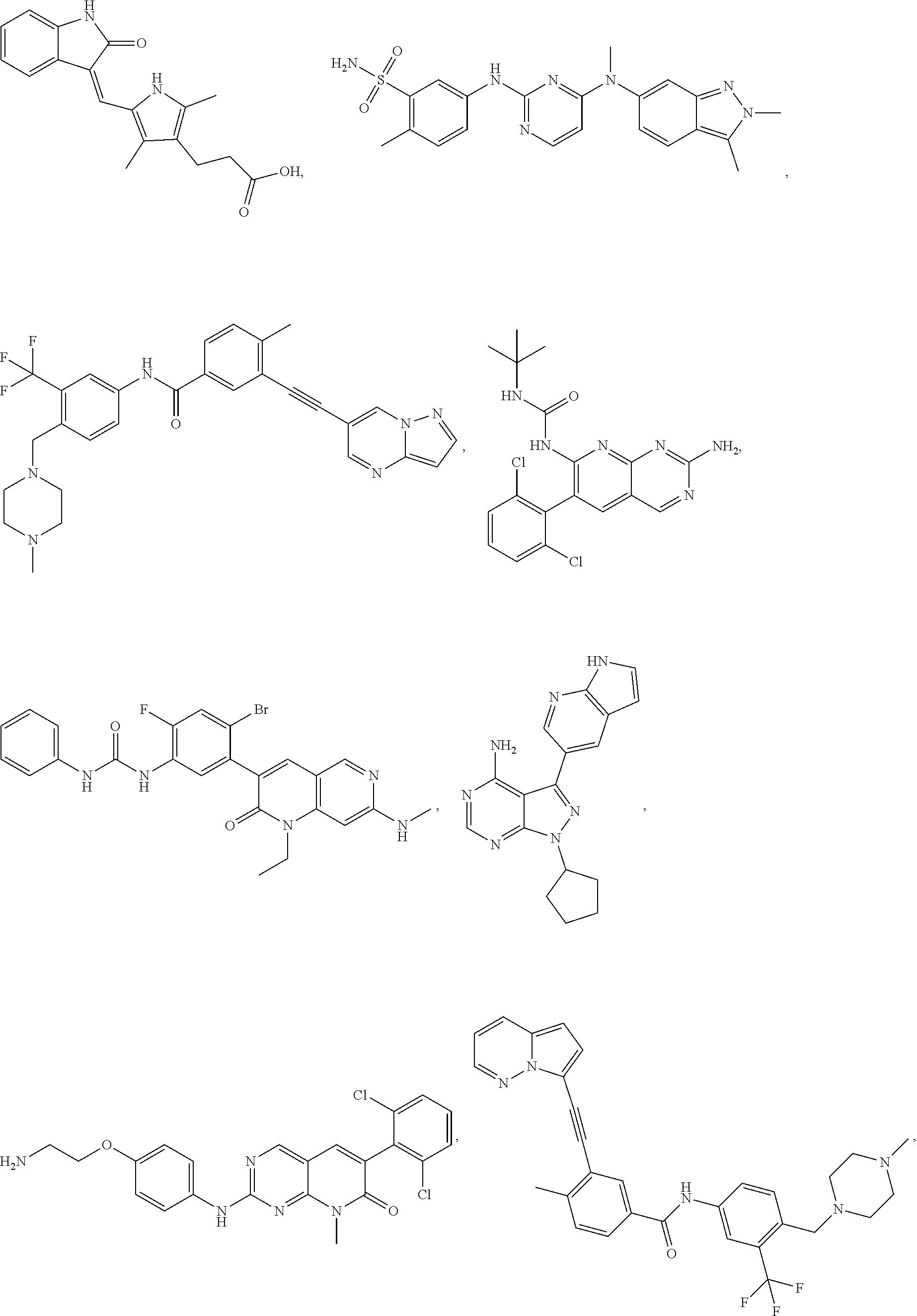 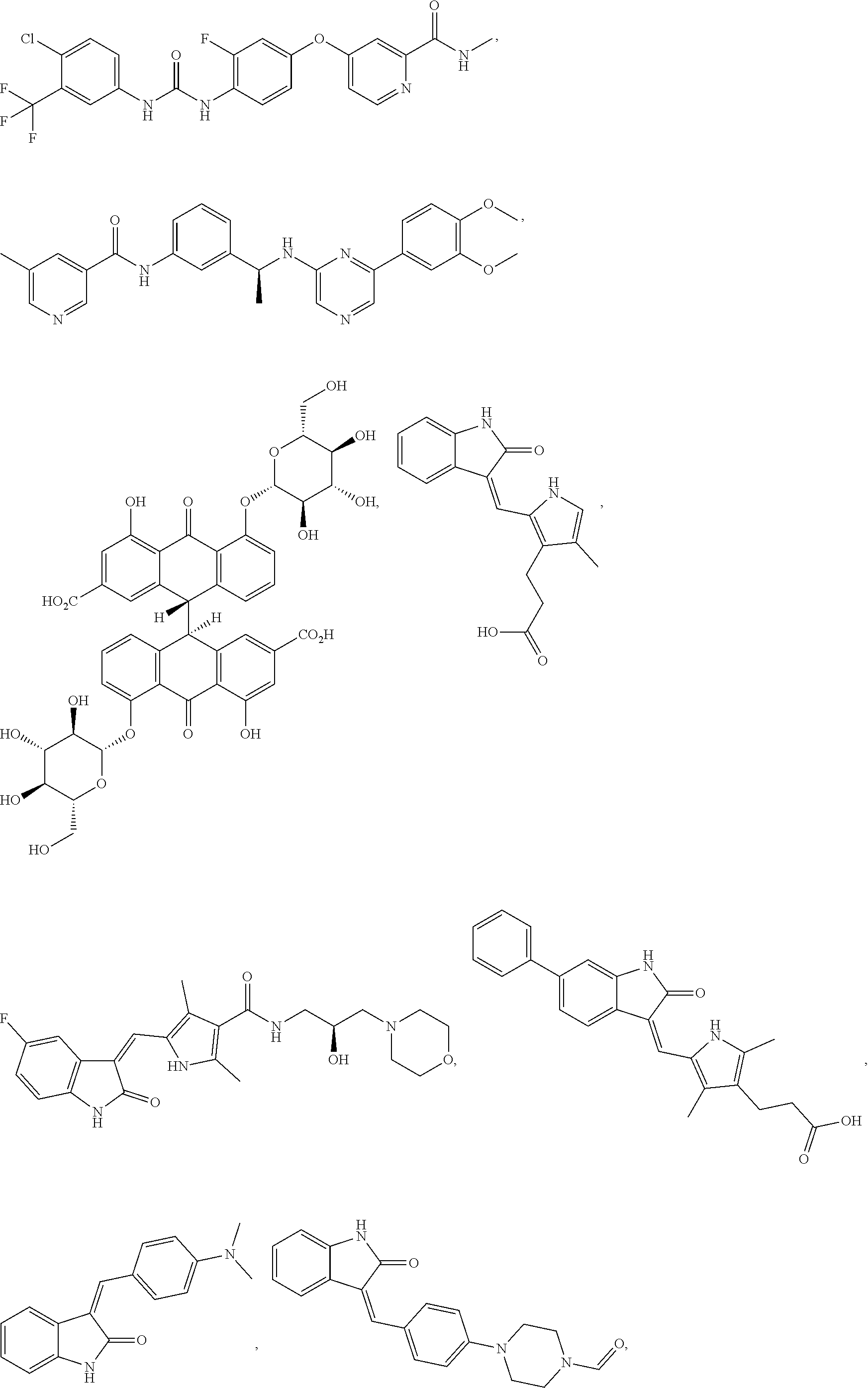 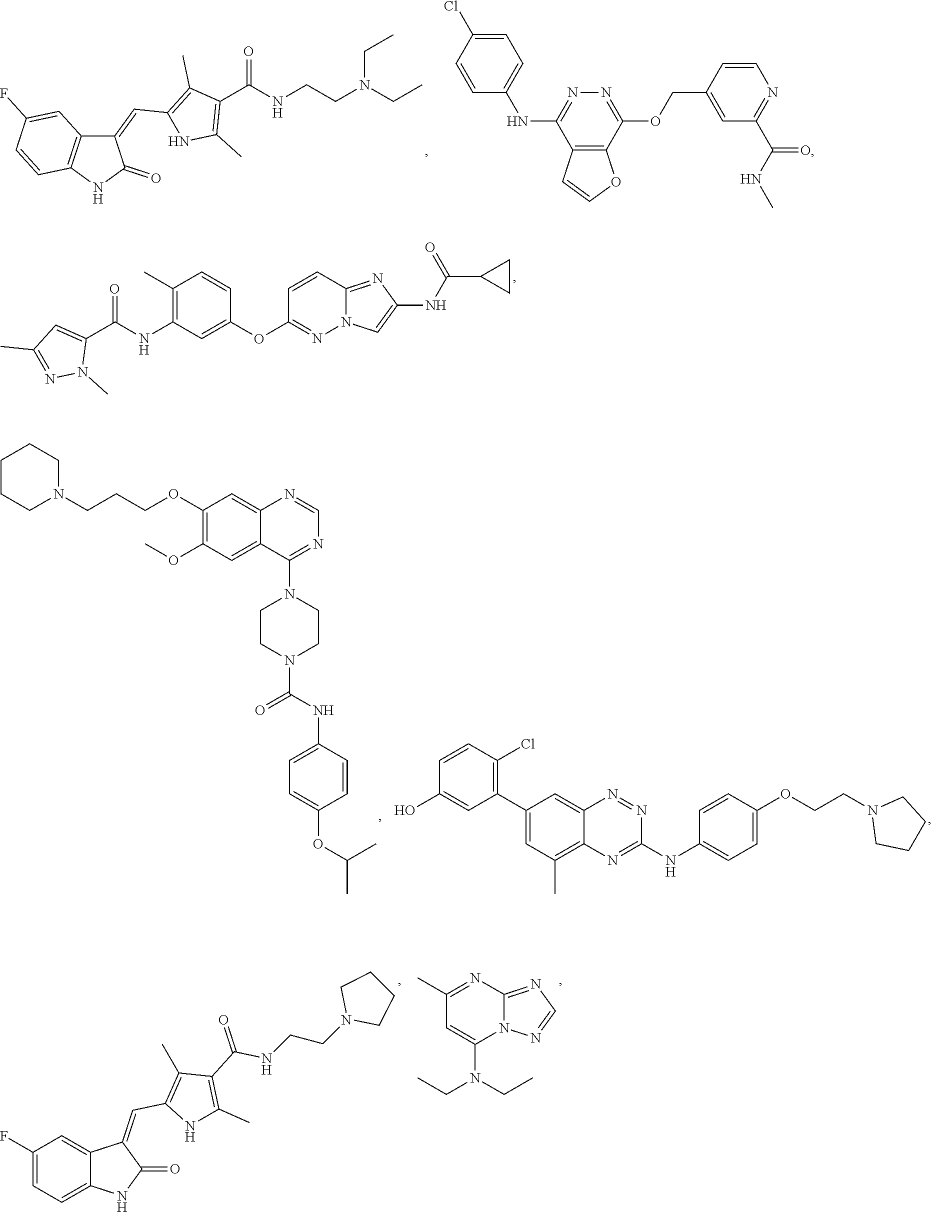 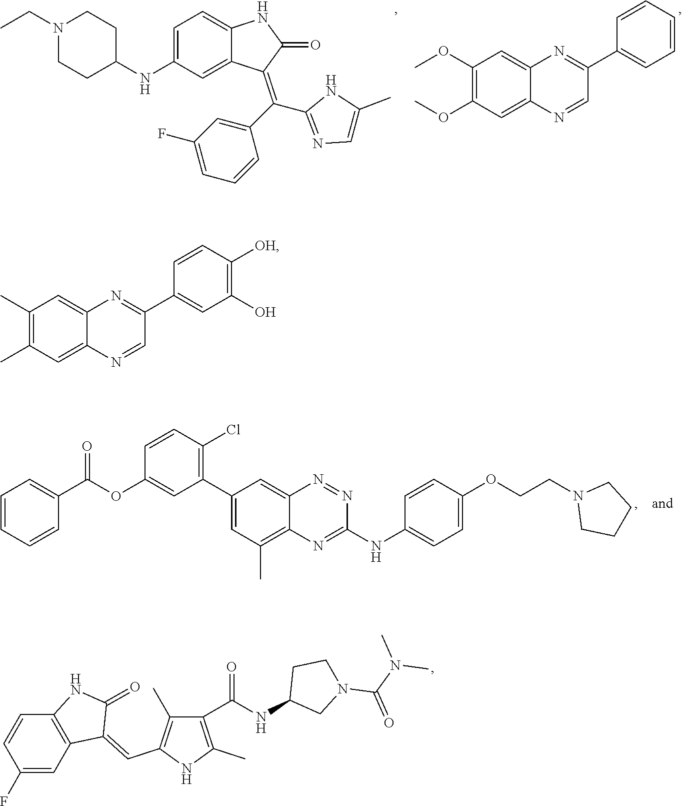
or a pharmaceutically acceptable salt thereof.

105. The method of claim 102, wherein the PDGFR inhibitor is a hydrate.

106. The method of claim 102, wherein the PDGFR inhibitor is selected from:

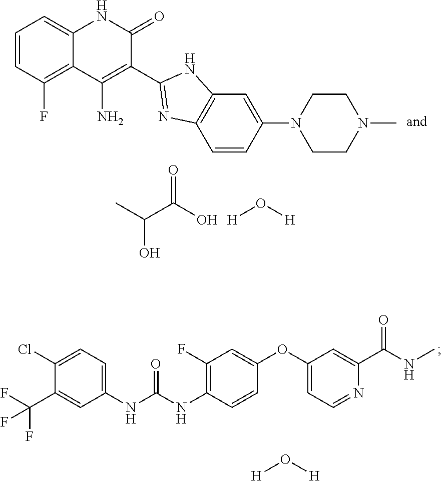

107. The method of claim 102, wherein the PDGFR inhibitor is an isotope, is deuterated, or is a deuterated isotope.

108. A method of enriching NO enteric neurons in a cell culture comprising exposing a composition of neural crest cells or a crestosphere with an effective amount of a PDGFR inhibitor or a pharmaceutically acceptable salt thereof.

109. The method of claim 108, wherein the cell culture comprises two-dimensional or three-dimensional neural crest cells.

110. The method of claim 108 wherein the step of exposing further comprises modulating nitric oxide responsiveness.

111. The method of claim 108, wherein the PDGFR inhibitor is chosen from: (Z)-orantinib, AC710, AC710 mesylate, AG 1295, amuvatinib, amuvatinib hydrochloride, avapritinib, axitinib, AZD2932, cediranib, cediranib maleate, chiauranib, CHIR-124, CP-673451, crenolanib, dovitinib, dovitinib lactate, dovitinib lactate hydrate, dovitinib-D8, ENMD-2076, ENMD-2076 tartrate, flumatinib, flumatinib mesylate, GZD856, GZD856 formic, HG-7-85-01, hypothemycrin, ilorasertib, ilorasertib hydrochloride, imatinib, imatinib D4, imatinib D8, imatinib mesylate, JI-101, JNJ-10198409, KG5, Ki20227, lenvatinib, lenvatinib mesylate, linifanib, masitinib, masitinib mesylate, methylnissolin, multi-kinase inhibitor 1, N-(p-coumaroyl) serotonin, nintedanib, nintedanib esylate, NVP-ACC789, orantinib, pazopanib, pazopanib hydrochloride, PD-089828, PD-161570, PDGFRα kinase inhibitor 1, ponatinib, ponatinib D8, PP121, PP58, regorafenib, regorafenib D3, regorafenib hydrochloride, regorafenib monohydrate, ripretinib, sennoside B, seralutinib, SU5402, SU14813, SU14813 maleate, SU16f, SU4312, SU4984, sunitinib, sunitinib D10, sunitinib malate, sunitinib-d4, TAK-593, tandutinib, tandutinib hydrochloride, telatinib, telatinib mesylate, TG 100572, TG 100572 hydrochloride, TG 100801, TG 100801 hydrochloride, toceranib, toceranib phosphate, toceranib-d8, trapidil, tyrosine kinase-IN-1, tyrphostin AG1296, tryphostin AG1433, vorolanib or a pharmaceutically acceptable salt.
